(19) United States
(12) Patent Application Publication
(10) Pub. No.: US 2026/0102501 A1
(43) Pub. Date: Apr. 16, 2026

(54) ANTI-CANCER NUCLEAR HORMONE RECEPTOR-TARGETING COMPOUNDS

(71) Applicant: Nuvation Bio Inc., New York, NY (US)

(72) Inventors: David HUNG, New York, NY (US); Christopher Paul MILLER, San Mateo, CA (US); Ihab S. DARWISH, San Carlos, CA (US)

(21) Appl. No.: 19/116,201

(22) PCT Filed: Sep. 28, 2023
(86) PCT No.: PCT/US2023/075456

§ 371 Date: Mar. 27, 2025

Related U.S. Application Data
(60) Provisional application No. 63/377,511, filed on Sep. 28, 2022.

(51) Int. Cl. A61K 47/54; A61K 47/55; A61P 35/00

(52) U.S. Cl. CPC A61K 47/545; A61K 47/552; A61K 47/554; A61P 35/00

(57) ABSTRACT

Provided herein are compounds comprising a nuclear payload, such as a topoisomerase inhibitor, topoisomerase poison, or analog thereof, and a nuclear receptor-targeting epitope.

### CROSS-REFERENCE TO RELATED APPLICATIONS

This application claims priority to and benefit of U.S. Provisional Patent Application No. 63/377,511, filed Sep. 28, 2022, the disclosure of which is hereby incorporated herein by reference in its entirety.

### BACKGROUND

Topoisomerase inhibitors are chemical compounds that block the action of topoisomerases, which are broken into two broad subtypes, type I topoisomerases (TopI) and type II topoisomerases (TopII). Topoisomerase plays important roles in cellular reproduction and DNA organization, as they mediate the cleavage of single and double stranded DNA to relax supercoils, untangle catenanes, and condense chromosomes in eukaryotic cells. Topoisomerase inhibitors influence these essential cellular processes. Some topoisomerase inhibitors prevent topoisomerases from performing DNA strand breaks, while others associate with topoisomerase-DNA complexes and prevent the re-ligation step of the topoisomerase mechanism. These topoisomerase-DNA-inhibitor complexes are cytotoxic agents, as the un-repaired single and double stranded DNA breaks that they cause can lead to apoptosis and cell death. Because of this ability to induce apoptosis, topoisomerase inhibitors have gained interest as therapeutics against infectious and cancerous cells.

Camptothecin (CPT) is a topoisomerase poison. It was isolated from the bark and stem of Camptotheca acuminata (Camptotheca, Happy tree), a tree native to China used as a cancer treatment in traditional Chinese medicine. CPT showed remarkable anticancer activity in preliminary clinical trials especially against breast, ovarian, colon, lung, and stomach cancers. However, it has low solubility and adverse effects have been reported when used therapeutically, so synthetic and medicinal chemists have developed numerous syntheses of camptothecin and various derivatives to increase the benefits of the chemical, with good results. Four CPT analogues have been approved and are used in cancer chemotherapy today, topotecan, irinotecan, belotecan, and trastuzumab deruxtecan. In addition to being an anti-tumor agent, camptothecin has also shown anti-HIV activity because it interrupts self-association of the viral-infectivity factor found in many retroviruses including HIV.

The future likely also holds many alternative uses for topoisomerase poisons, including lupus, rare brain disorders, sepsis, and viral and trypanosomal infections. As additional roles of Top1 (such as newly-discovered regulatory functions) emerge, and Top1 continues to be implicated in disease states, new drug discovery (and drug repurposing) efforts will continue for years to come.

### SUMMARY

Provided herein are compounds comprising a nuclear payload, such as a topoisomerase inhibitor, topoisomerase poison, or analog thereof, and a nuclear receptor-targeting epitope. Compounds described herein are designed to bind nuclear receptors within the cell and allow the compound, with its nuclear payload, to accumulate in the nucleus. Not wishing to be bound by theory, one potential mode of enhanced utility is that this approach may provide for compounds having cell-type selectivity, not merely improved potency, working toward a higher therapeutic index. However, it may be that the compounds may be active by other modes, such as, but not limited to, passive localization in the nucleus.

Further, the compounds described herein offer targeted delivery of a nuclear payload. The compounds both target and localize within tumor tissue. The transport of the compound, which comprises at least one nuclear receptor-targeting epitope, such as a nuclear steroid receptor-targeting epitope, covalently attached to at least one nuclear payload, to the nucleus allows for accumulation of the nuclear payload in the nucleus, enhancing tumor cell death. By doing so, compounds described in this disclosure may exhibit superior efficacy. In addition, the compounds described in this disclosure will, by accumulating in the nucleus of nuclear receptor positive cells, such as steroid receptor positive cells, spare cells that do not express the specific nuclear steroid receptor, and therefore reduce side effects.

In certain embodiments, provided is a compound of Formula I, or stereoisomer, mixture of stereoisomers, hydrate, solvate, isotopically enriched analog or pharmaceutically acceptable salt thereof:

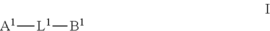

wherein:
B¹ is a nuclear receptor-targeting epitope;
L¹ is a covalent bond or a linking moiety; and
A¹ is of Formula IA:

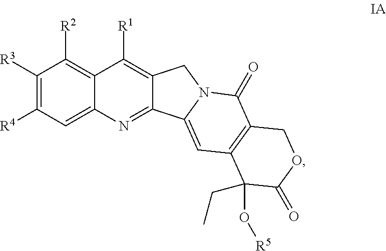

wherein:
R¹, R², R³, R⁴, and R⁵ are each independently hydrogen, halo, cyano, nitro, —OR¹⁵, —SR¹⁵, —NR¹⁵R¹⁶, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁵, —C(═O)OR¹⁵, —OC(═O)R¹⁵, —OC(═O)NR¹⁵R¹⁶, —C(═O)NR¹⁵R¹⁶, —NR¹⁵C(═O)R¹⁶, —NR¹⁵C(═O)OR¹⁶, —S(═O)₁₋₂R¹⁵, —S(═O)₁₋₂NR¹⁵R¹⁶, —NR¹⁵S(═O)₁₋₂R¹⁶, —Si(R¹⁵)₃, or —C═NOR¹⁵, each independently optionally substituted with one or more R¹⁰ as valency permits;
   or R¹ and R² are taken together with the atoms to which they are attached to form a C₃₋₁₂ cycloalkyl, 5- to 12-membered heterocyclyl, C₆₋₁₂ aryl, or 5- to 12-membered heteroaryl, each independently optionally substituted with one or more R¹⁰ as valency permits;
   or R² and R³ are taken together with the atoms to which they are attached to form a C₃₋₁₂ cycloalkyl, 5- to 12-membered heterocyclyl, C₆₋₁₂ aryl, or 5- to 12-membered heteroaryl, each independently optionally substituted with one or more R¹⁰ as valency permits;
   or R³ and R⁴ are taken together with the atoms to which they are attached to form a C₃₋₁₂ cycloalkyl, 5- to 12-membered heterocyclyl, C₆₋₁₂ aryl, or 5- to 12-membered heteroaryl, each independently optionally substituted with one or more R¹⁰ as valency permits;
each R¹⁰ is independently halo, cyano, nitro, —OR¹⁷, —SR¹⁷, —SF₅, —NR¹⁷R¹⁸, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷, —C(═O)OR¹⁷, —OC(═O)OR¹⁷, —OC(═O)R¹⁷, —C(═O)NR¹⁷R¹⁸, —OC(═O)NR¹⁷R¹⁸, —NR⁷C(═O)NR¹⁷R¹⁸, —S(═O)₁₋₂R¹⁷, —S(═O)₁₋₂NR¹⁷R¹⁸, —NR¹⁷S(═O)₁₋₂R¹⁸, —NR¹⁷S(═O)₁₋₂NR¹⁷R¹⁸, —NR¹⁷C(═O)R¹⁸, —NR¹⁷C(═O)OR¹⁸, —Si(R¹⁷)₃, or —C═NOR¹⁷, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
each of R¹⁵ and R¹⁶ is independently hydrogen, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, or 5- to 12-membered heteroaryl, each independently optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
   or R¹⁵ and R¹⁶ are taken together with the atoms to which they are attached to form 5- to 12-membered heterocyclyl optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each R¹⁷ and R¹⁸ is independently hydrogen, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, or 5- to 12-membered heteroaryl, each independently optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
   or R¹⁷ and R¹⁸ are taken together with the atoms to which they are attached to form 5- to 12-membered heterocyclyl optionally substituted with one or more substituents selected from the group consisting of halo, cyano, nitro, hydroxyl, amino, C₁₋₁₂ alkoxyl, C₁-12 alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
wherein one or more atoms of Formula IA (e.g., hydrogen, methyl, or hydroxyl) is replaced by a direct covalent bond to L¹.

Also provided is a compound of Table 1, or stereoisomer, mixture of stereoisomers, hydrate, solvate, isotopically enriched analog, or pharmaceutically acceptable salt thereof.

Also provided is a composition comprising a compound as described herein or stereoisomer, mixture of stereoisomers, hydrate, solvate, isotopically enriched analog, or pharmaceutically acceptable salt thereof, and a pharmaceutically acceptable excipient.

Also provided is a method of treating or preventing cancer, comprising administering an effective amount of a compound or composition as described herein to an individual in need thereof. The cancer can be a blood cancer, lung cancer, breast cancer, fallopian tube cancer, brain cancer, head and neck cancer, esophageal cancer, ovarian cancer, pancreatic cancer, peritoneal cancer, prostate cancer or skin cancer, such as, but not limited to, liver cancer, melanoma, Hodgkin's disease, non-Hodgkin's lymphomas, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, Waldenström macroglobulinemia, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, malignant melanoma, choriocarcinoma, mycosis fungoides, head neck carcinoma, osteogenic sarcoma, pancreatic carcinoma, acute granulocytic leukemia, hairy cell leukemia, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, trophoblastic neoplasms, or prostatic carcinoma.

Also provided is a method of treating or preventing a cancer, comprising administering an effective amount of a compound or composition as described herein to an individual in need thereof.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### 1. Definitions

As used in the present specification, the following words, phrases and symbols are generally intended to have the meanings as set forth below, except to the extent that the context in which they are used indicates otherwise.

The term “about” refers to a variation of ±1%, ±3%, ±5%, or ±10% of the value specified. For example, “about 50” can in some embodiments includes a range of from 45 to 55. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. Also, the singular forms “a” and “the” include plural references unless the context clearly dictates otherwise. Thus, e.g., reference to “the compound” includes a plurality of such compounds and reference to “the assay” includes reference to one or more compounds and equivalents thereof known to those skilled in the art.

“Alkyl” refers to an unbranched or branched saturated hydrocarbon chain. As used herein, alkyl has 1 to 12 carbon atoms (a “C₁₋₁₂ alkyl”), 1 to 10 carbon atoms (i.e., C₁₋₁₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NH—, —O—, —S—, —S(O)—, —S(O)₂—, and the like. As used herein, heteroalkyl includes 1 to 8 carbon atoms, or 1 to 4 carbon atoms; and 1 to 3 heteroatoms, 1 to 2 heteroatoms, or 1 heteroatom. “Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NH—, —O—, —S—, —S(O)—, —S(O)₂—. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.), and amines (e.g., —CH₂NHCH₃, —CH(CH₃)NHCH₃, —CH₂CH₂NHCH₃, —CH₂CH₂NHCH₂CH₂NHCH₃, etc. As used herein, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom.

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl) or 2 to 4 carbon atoms (i.e., C₂₋₄ alkenyl). Examples of alkenyl groups include, e.g., ethenyl, propenyl, and butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl) or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Alkoxyalkyl” refers to the group “alkyl-O-alkyl”.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, thiophenyl (i.e., thienyl), triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more (e.g., 1 to 3) oxo (═O) or N-oxide (—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. The term “heterocyclyl” also includes “spiroheterocyclyl” when there are two positions for substitution on the same carbon atom. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxctanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Alkylene” refers to a divalent alkyl group as defined above.

“Alkenylene” refers to a divalent alkenyl group as defined above.

“Alkynylene” refers to a divalent alkynyl group as defined above.

“Arylene” refers to a divalent aryl group as defined above.

“Cycloalkylene” refers to a divalent cycloalkyl group as defined above.

“Heterocyclylene” refers to a divalent heterocyclyl group as defined above.

“Heteroarylene” refers to a divalent heteroaryl group as defined above.

“Oxo” refers to ═O.

“Halogen” or “halo” includes fluoro, chloro, bromo, and iodo.

The terms “optional” or “optionally” means that the subsequently described event or circumstance may or may not occur. The term “optionally substituted” refers to any one or more hydrogen atoms on the designated atom or group may or may not be replaced by a moiety other than hydrogen.

“Substituted” as used herein means one or more hydrogen atoms of the group is replaced with a substituent atom or group commonly used in pharmaceutical chemistry. Each substituent can be the same or different. Examples of suitable substituents include, but are not limited to, hydrazide, halo, —CN, —NO₂, alkyl, alkenyl, alkynyl, cycloalkyl, aryl, heterocyclyl, heteroaryl, —OR⁵⁶, —C(O)OR⁵⁶, —C(O)R⁵⁶, —O-alkyl-OR⁵⁶, -alkyl-OR⁵⁶, haloalkyl, haloalkoxy, SR⁵⁶, S(O)R⁵⁶, SO₂R⁵⁶, NR⁵⁶R⁵⁷, —C(O)NR⁵⁶R⁵⁷, NR⁵⁶C(O)R⁵⁷, including seleno and thio derivatives thereof, wherein each R⁵⁶ and R⁵⁷ are independently hydrogen, alkyl, alkenyl, alkynyl, heteroalkyl, cycloalkyl, cycloalkyl-alkyl-, heterocyclyl, heterocyclyl-alkyl-, aryl, aryl-alkyl-, heteroaryl, or heteroaryl-alkyl-, and wherein each of the substituents can be optionally further substituted.

Provided are also stereoisomers, mixture of stereoisomers, tautomers, hydrates, solvates, isotopically enriched analog, and pharmaceutically acceptable salts of the compounds described herein.

The compounds disclosed herein, or their pharmaceutically acceptable salts, may include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high performance liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposeable mirror images of one another and “diastereomers,” which refers to stereoisomers that have at least two asymmetric atoms, but which are not mirror-images of each other. Thus, all stereoisomers (for example, geometric isomers, optical isomers, and the like) of the present compounds (including those of the salts, solvates, and hydrates of the compounds), such as those which may exist due to asymmetric carbons on various substituents, including enantiomeric forms (which may exist even in the absence of asymmetric carbons), rotameric forms, atropisomers, and diastereomeric forms, are contemplated.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds may be atropisomers and are considered as part of this disclosure. Stereoisomers can also be separated by use of chiral HPLC.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid, and ethanolamine.

Any compound or structure given herein, is also intended to represent unlabeled forms as well as isotopically labeled forms of the compounds. These forms of compounds may also be referred to as an “isotopically enriched analog.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Such compounds may exhibit increased resistance to metabolism and are thus useful for increasing the half-life of any compound when administered to a mammal, particularly a human. Such compounds are synthesized by means well known in the art, for example by employing starting materials in which one or more hydrogens have been replaced by deuterium.

Certain compounds disclosed herein contain one or more ionizable groups (groups from which a proton can be removed (e.g., —COOH) or added (e.g., amines) or which can be quaternized (e.g., amines)). All possible ionic forms of such molecules and salts thereof are intended to be included individually in the disclosure herein. With regard to salts of the compounds described herein, one of ordinary skill in the art can select from among a wide variety of available counterions those that are appropriate. In specific applications, the selection of a given anion or cation for preparation of a salt may result in increased or decreased solubility of that salt.

As used herein, the term “non-biocleavable linking moiety” is intended to refer to a linking moiety which is not readily hydrolyzed under physiological conditions. As used herein, the term “biocleavable linking moiety” is intended to refer to a linking moiety which is readily hydrolyzed under physiological conditions. In certain embodiments, at least one linking moiety is hydrolyzed under intracellular conditions (e.g., low pH). In some embodiments, the biocleavable is self-cleaving and does not require physiological hydrolysis, in other embodiments, the biocleavable linker's cleavage is initiated by metabolic activation such as oxidation or pH dependent cleavage without hydrolysis such as by base or acid induced elimination, etc. More broadly speaking, a biocleavable linker may in some instances be analogous to a prodrug wherein after cleavage, one or more drugs is released. In this sense, there are many mechanisms to cleave prodrugs and release the active(s) or a precursor that yields the active and there are also many varieties of cleavage moieties known in the prodrug art and are included in our definition herein.

As used herein, the term “cancer” refers to a class of diseases of mammals characterized by uncontrolled cellular growth. The term “cancer” is used interchangeably with the terms “tumor,” “solid tumor,” “malignancy,” “hyperproliferation,” and “neoplasm.” Cancer includes all types of hyperproliferative growth, hyperplasic growth, neoplastic growth, cancerous growth, or oncogenic processes, metastatic tissues or malignantly transformed cells, tissues, or organs, irrespective of histopathologic type or stage of invasiveness. Illustrative examples include, lung, prostate, head and neck, breast and colorectal cancer, melanomas and gliomas (such as a high grade glioma, including glioblastoma multiforme (GBM), the most common and deadliest of malignant primary brain tumors in adult humans).

The phrase “solid tumor” includes, for example, lung cancer, head and neck cancer, brain cancer, oral cancer, colorectal cancer, breast cancer, prostate cancer, pancreatic cancer, and liver cancer. Other types of solid tumors are named for the particular cells that form them, for example, sarcomas formed from connective tissue cells (for example, bone cartilage, fat), carcinomas formed from epithelial tissue cells (for example, breast, colon, pancreas), and lymphomas formed from lymphatic tissue cells (for example, lymph nodes, spleen, and thymus). Treatment of all types of solid tumors regardless of naming convention is within the scope of this disclosure.

“Chemotherapeutic agent” refers to any substance capable of reducing or preventing the growth, proliferation, or spread of a cancer cell, a population of cancer cells, tumor, or other malignant tissue. The term is intended also to encompass radiotherapy, or any antitumor or anticancer agent.

As used herein, “treatment” or “treating” is an approach for obtaining a beneficial or desired result, such as a clinical result. For purposes of this disclosure, beneficial or desired clinical results include, but are not limited to, alleviation of a symptom and/or diminishment of the extent of a symptom and/or preventing a worsening of a symptom associated with a disease or condition. In one variation, beneficial or desired clinical results include, but are not limited to, alleviation of a symptom and/or diminishment of the extent of a symptom and/or preventing a worsening of a symptom associated with a cognitive disorder, a psychotic disorder, a neurotransmitter-mediated disorder and/or a neuronal disorder. In one embodiment, treatment of a disease or condition with a compound of the disclosure or a pharmaceutically acceptable salt thereof is accompanied by no or fewer side effects than are associated with currently available therapies for the disease or condition and/or improves the quality of life of the individual.

The terms “inhibit,” “inhibiting.” and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

As used herein, by “combination therapy” is meant a therapy that includes two or more different compounds. Thus, in one aspect, a combination therapy comprising a compound detailed herein and anther compound is provided. In some variations, the combination therapy optionally includes one or more pharmaceutically acceptable carriers or excipients, non-pharmaceutically active compounds, and/or inert substances. In various embodiments, treatment with a combination therapy may result in an additive or even synergistic (e.g., greater than additive) result compared to administration of a single compound of the disclosure alone. In some embodiments, a lower amount of each compound is used as part of a combination therapy compared to the amount generally used for individual therapy. In one embodiment, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a compound in a combination therapy than the amount generally used for individual compound or therapy. Preferably, the use of a small amount of compound results in a reduction in the number, severity, frequency, and/or duration of one or more side-effects associated with the compound.

As used herein, the term “effective amount” intends such amount of a compound of the disclosure which in combination with its parameters of efficacy and toxicity, as well as based on the knowledge of the practicing specialist should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

As used herein, the IC₅₀ refers to an amount, concentration or dosage of a particular test compound that achieves a 50% inhibition of a maximal response, such as modulation of PARP, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

The term “cancer,” as used herein refers to an abnormal growth of cells which tend to proliferate in an uncontrolled way and, in some cases, to metastasize (spread). The types of cancer include, but are not limited to, solid tumors (such as those of the bladder, bowel, brain, breast, endometrium, heart, kidney, lung, lymphatic tissue (lymphoma), ovary, pancreas or other endocrine organ (thyroid)), prostate, skin (melanoma) or hematological tumors (such as the leukemias).

The term “carrier,” as used herein, refers to relatively nontoxic chemical compounds or agents that facilitate the incorporation of a compound into cells or tissues.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid, and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, and the like. Acceptable inorganic bases include aluminum hydroxide, calcium hydroxide, potassium hydroxide, sodium carbonate, sodium hydroxide, and the like. Further examples of pharmaceutically acceptable salts include those listed in Berge et al., Pharmaceutical Salts, J. Pharm. Sci. 1977 January; 66(1):1-19. Pharmaceutically acceptable salts can be prepared in situ in the manufacturing process, or by separately reacting a purified compound of the disclosure in its free acid or base form with a suitable organic or inorganic base or acid, respectively, and isolating the salt thus formed during subsequent purification. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The term “excipient” as used herein means an inert or inactive substance that may be used in the production of a drug or pharmaceutical, such as a tablet containing a compound of the disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (directly compressible), honey dc, lactose (anhydrate or monohydrate; optionally in combination with aspartame, cellulose, or microcrystalline cellulose), starch de, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose de, etc.; and wet granulation agents include, e.g., calcium carbonate, maltodextrin, microcrystalline cellulose, etc.

### Compounds

Provided herein are targeted compounds for treating cancer. The compounds described herein are capable of targeting the nucleus of a cell by recognition and binding of a nuclear receptor-targeting epitope to the respective binding site and delivering the nuclear payload to the nucleus of the cell. The nuclear payload then is capable of binding to one or more target sites within the nucleus and/or disrupting one or more cellular processes, causing the cell to die.

In certain embodiments, the nuclear payload is bonded to the nuclear receptor-targeting epitope(s) via a linking moiety. In certain embodiments, the linking moiety provides a single or mono-linkage, meaning that the linker is only conjugated to one atom of each of the payload and the epitope.

Accordingly, provided is a compound of Formula I, or stereoisomer, mixture of stereoisomers, hydrate, solvate, isotopically enriched analog, or pharmaceutically acceptable salt thereof:

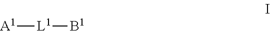

wherein:
B¹ is a nuclear receptor-targeting epitope;
L¹ is a covalent bond or a linking moiety; and
A¹ is of Formula IA:

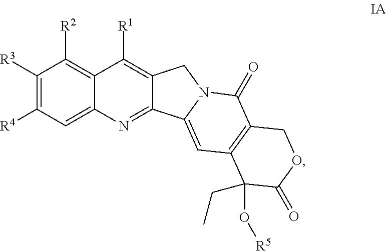

wherein:
R¹, R², R³ and R⁴ are each independently hydrogen, halo, cyano, nitro, —OR¹⁵, —SR¹⁵, —NR¹⁵R¹⁶, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁵, —C(═O)OR¹⁵, —OC(═O)R¹⁵, —OC(═O)NR¹⁵R¹⁶, —C(═O)NR¹⁵R¹⁶, —NR¹⁵C(═O)R¹⁶, —NR¹⁵C(═O)OR¹⁶, —S(═O)₁₋₂R¹⁵, —S(═O)₁₋₂NR¹⁵R¹⁶, —NR¹⁵S(═O)₁₋₂R¹⁶, —Si(R¹⁵)₃, or —C═NOR¹⁵, each independently optionally substituted with one or more R¹⁰ as valency permits;
   or R¹ and R² are taken together with the atoms to which they are attached to form a C₃₋₁₂ cycloalkyl, 5- to 12-membered heterocyclyl, C₆₋₁₂ aryl, or 5- to 12-membered heteroaryl, each independently optionally substituted with one or more R¹⁰ as valency permits;
   or R² and R³ are taken together with the atoms to which they are attached to form a C₃₋₁₂ cycloalkyl, 5- to 12-membered heterocyclyl, C₆₋₁₂ aryl, or 5- to 12-membered heteroaryl, each independently optionally substituted with one or more R¹⁰ as valency permits;
   or R³ and R⁴ are taken together with the atoms to which they are attached to form a C₃₋₁₂ cycloalkyl, 5- to 12-membered heterocyclyl, C₆₋₁₂ aryl, or 5- to 12-membered heteroaryl, each independently optionally substituted with one or more R¹⁰ as valency permits;
R⁵ is hydrogen or —C(═O)R¹⁵;
each R¹⁰ is independently halo, cyano, nitro, —OR¹⁷, —SR¹⁷, —SF₅, —NR¹⁷R¹⁸, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷, —C(═O)OR¹⁷, —OC(═O)OR¹⁷, —OC(═O)R¹⁷, —C(═O)NR¹⁷R¹⁸, —OC(═O)NR¹⁷R¹⁸, —NR⁷C(═O)NR¹⁷R¹⁸, —S(═O)₁₋₂R¹⁷, —S(═O)₁₋₂NR¹⁷R¹⁸, —NR¹⁷S(═O)₁₋₂R¹⁸, —NR¹⁷S(═O)₁₋₂NR¹⁷R¹⁸, —NR¹⁷C(═O)R¹⁸, —NR¹⁷C(═O)OR¹⁸, —Si(R¹⁷)₃, or —C═NOR¹⁷, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
each of R¹⁵ and R¹⁶ is independently hydrogen, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, or 5- to 12-membered heteroaryl, each independently optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
   or R¹⁵ and R¹⁶ are taken together with the atoms to which they are attached to form 5- to 12-membered heterocyclyl optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each R¹⁷ and R¹⁸ is independently hydrogen, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, or 5- to 12-membered heteroaryl, each independently optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
   or R¹⁷ and R¹⁸ are taken together with the atoms to which they are attached to form 5- to 12-membered heterocyclyl optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
wherein one or more atoms of Formula IA (e.g., hydrogen, methyl, or hydroxyl) is replaced by a direct covalent bond to L¹.

In certain embodiments, the compound is not a compound selected from the group of compounds in Table 1X, or stereoisomer, mixture of stereoisomers, hydrate, solvate, isotopically enriched analog or pharmaceutically acceptable salt thereof.

TABLE 1X

| No. | Structure |
|---|---|
| 1X | 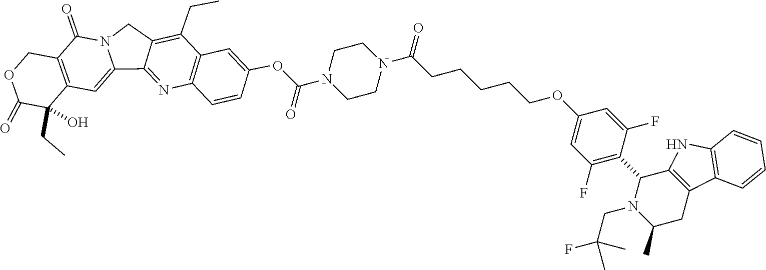 |
| 2X | 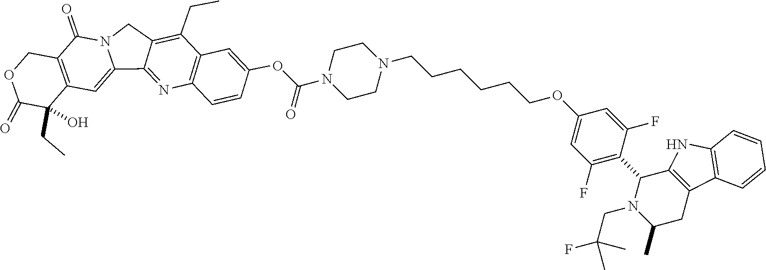 |
| 3X | 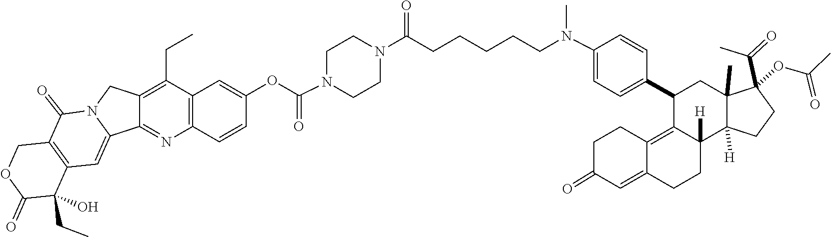 |
| 4aX | 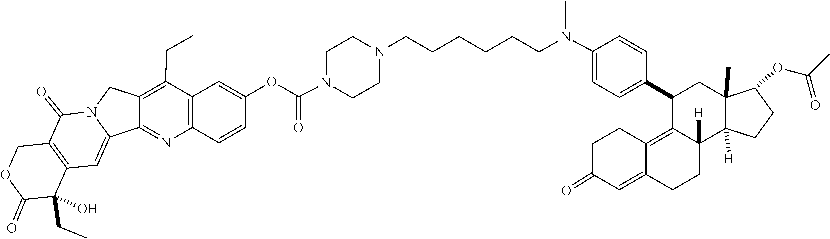 |
| 4X | 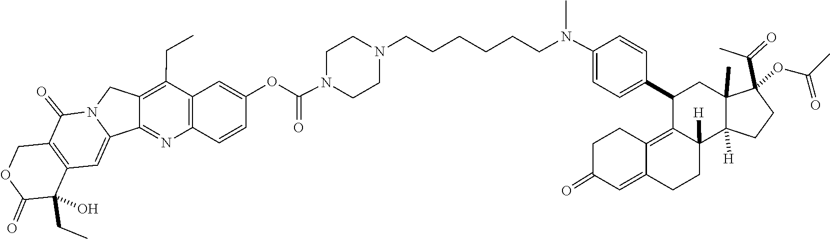 |
| 5X | 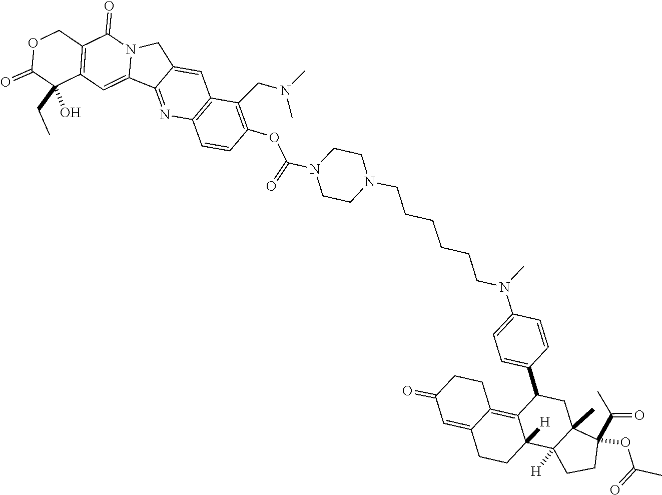 |
| 6X | 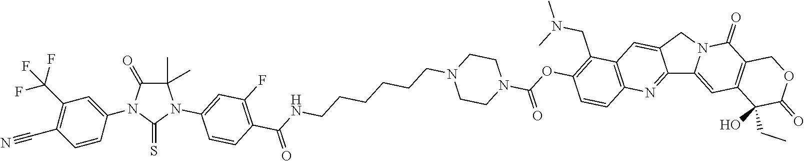 [SMILES: CC[C@@]1(O)C(=O)OCc2c1cc1n(c2=O)Cc2cc3c(CN(C)C)c(OC(=O)N4CCN(CCCCCCNC(=O)c5ccc(N6C(=S)N(c7ccc(C#N)c(C(F)(F)F)c7)C(=O)C6(C)C)cc5F)CC4)ccc3nc2-1] |
| 7X | 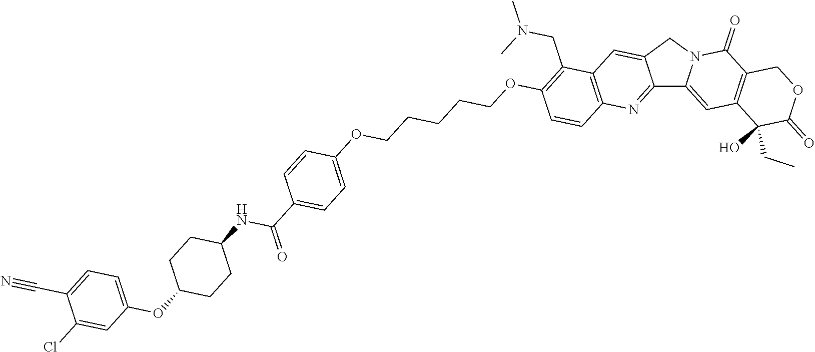 [SMILES: CC[C@@]1(O)C(=O)OCc2c1cc1n(c2=O)Cc2cc3c(CN(C)C)c(OCCCCCOc4ccc(C(=O)N[C@H]5CC[C@H](Oc6ccc(C#N)c(Cl)c6)CC5)cc4)ccc3nc2-1] |
| 8X | 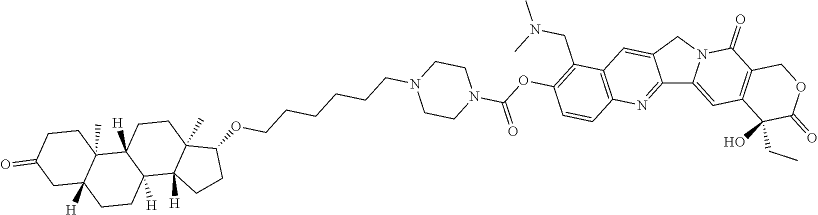 [SMILES: CC[C@@]1(O)C(=O)OCc2c1cc1n(c2=O)Cc2cc3c(CN(C)C)c(OC(=O)N4CCN(CCCCCCO[C@@H]5CC[C@@H]6[C@H]7CC[C@@H]8CC(=O)CC[C@@]8(C)[C@@H]7CC[C@]65C)CC4)ccc3nc2-1] |
| 9X | 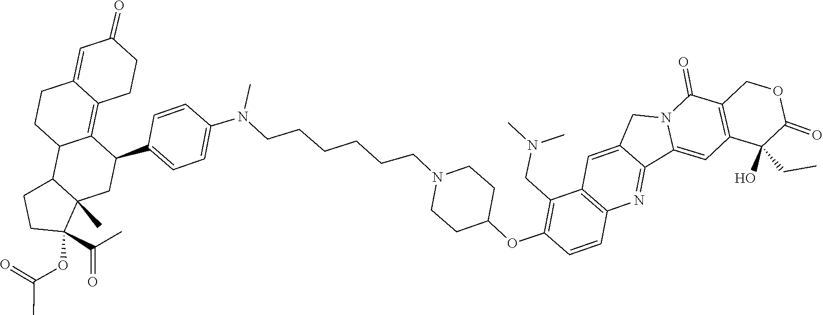 [SMILES: CC[C@@]1(O)C(=O)OCc2c1cc1n(c2=O)Cc2cc3c(CN(C)C)c(OC4CCN(CCCCCCN(C)c5ccc([C@H]6C[C@@]7(C)C(CC[C@]7(OC(C)=O)C(C)=O)C7CCC8=CC(=O)CCC8=C76)cc5)CC4)ccc3nc2-1] |
| 10X | 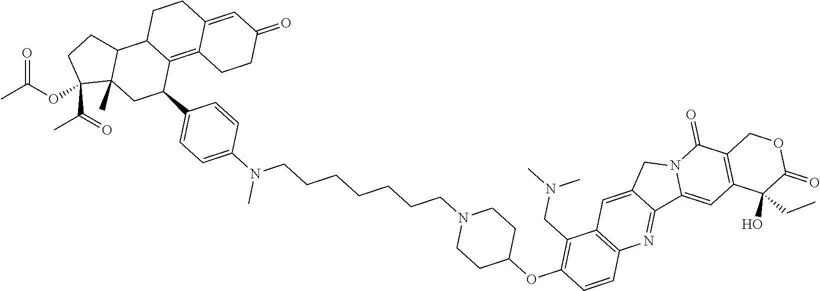 [SMILES: CC[C@@]1(O)C(=O)OCc2c1cc1n(c2=O)Cc2cc3c(CN(C)C)c(OC4CCN(CCCCCCCN(C)c5ccc([C@H]6C[C@@]7(C)C(CC[C@]7(OC(C)=O)C(C)=O)C7CCC8=CC(=O)CCC8=C76)cc5)CC4)ccc3nc2-1] |
| 11X | 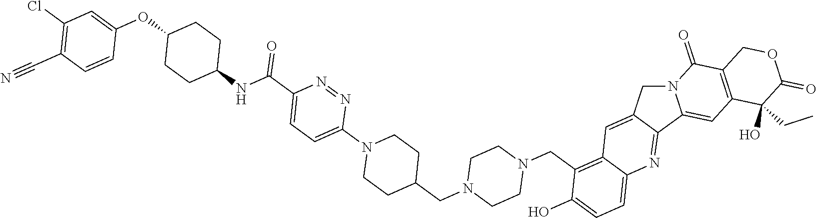 [SMILES: CC[C@@]1(O)C(=O)OCc2c1cc1n(c2=O)Cc2cc3c(CN4CCN(CC5CCN(c6ccc(C(=O)N[C@H]7CC[C@H](Oc8ccc(C#N)c(Cl)c8)CC7)nn6)CC5)CC4)c(O)ccc3nc2-1] |
| 12X | 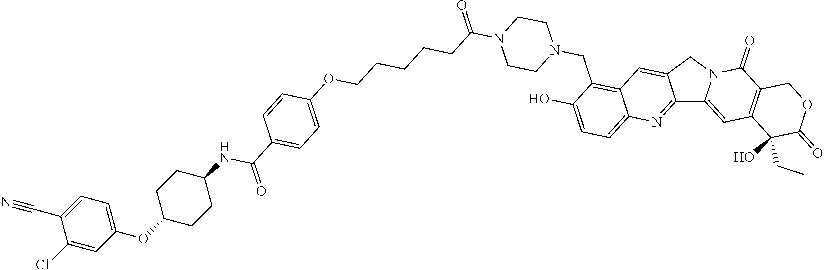 [SMILES: CC[C@@]1(O)C(=O)OCc2c1cc1n(c2=O)Cc2cc3c(CN4CCN(C(=O)CCCCCOc5ccc(C(=O)N[C@H]6CC[C@H](Oc7ccc(C#N)c(Cl)c7)CC6)cc5)CC4)c(O)ccc3nc2-1] |
| 13X | 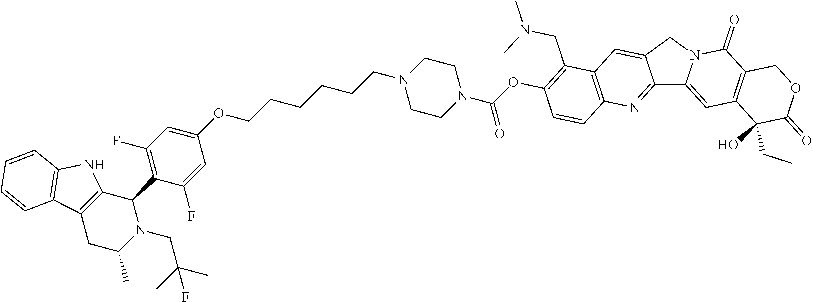 [SMILES: CC[C@@]1(O)C(=O)OCc2c1cc1n(c2=O)Cc2cc3c(CN(C)C)c(OC(=O)N4CCN(CCCCCCOc5cc(F)c([C@@H]6c7[nH]c8ccccc8c7C[C@@H](C)N6CC(C)(C)F)c(F)c5)CC4)ccc3nc2-1] |
| 14X | 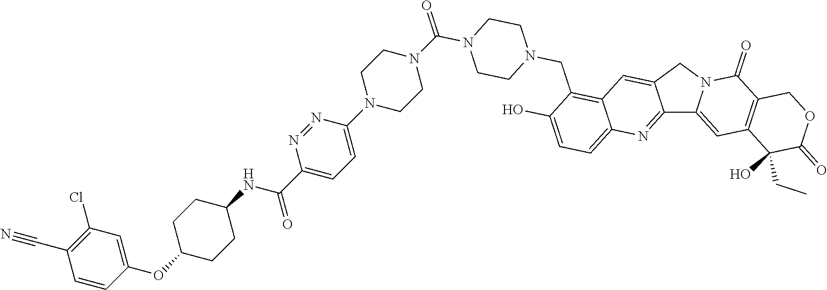 [SMILES: CC[C@@]1(O)C(=O)OCc2c1cc1n(c2=O)Cc2cc3c(CN4CCN(C(=O)N5CCN(c6ccc(C(=O)N[C@H]7CC[C@H](Oc8ccc(C#N)c(Cl)c8)CC7)nn6)CC5)CC4)c(O)ccc3nc2-1] |
| 15X | 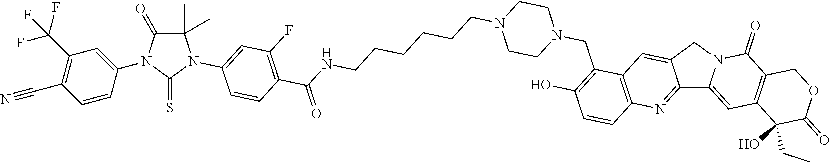 [SMILES: CC[C@@]1(O)C(=O)OCc2c1cc1n(c2=O)Cc2cc3c(CN4CCN(CCCCCCNC(=O)c5ccc(N6C(=S)N(c7ccc(C#N)c(C(F)(F)F)c7)C(=O)C6(C)C)cc5F)CC4)c(O)ccc3nc2-1] |
| 16X | 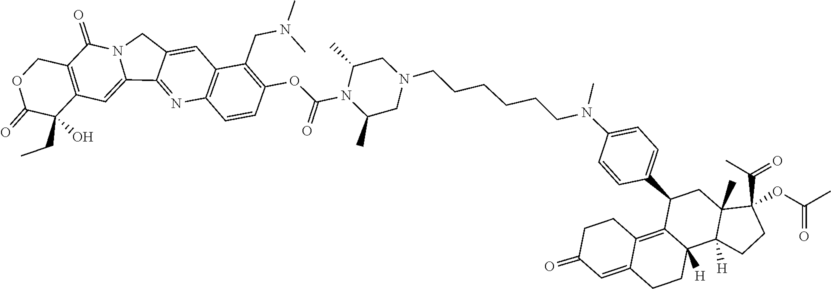 [SMILES: CC[C@@]1(O)C(=O)OCc2c1cc1n(c2=O)Cc2cc3c(CN(C)C)c(OC(=O)N4[C@H](C)CN(CCCCCCN(C)c5ccc([C@H]6C[C@@]7(C)[C@@H](CC[C@]7(OC(C)=O)C(C)=O)[C@@H]7CCC8=CC(=O)CCC8=C76)cc5)C[C@H]4C)ccc3nc2-1] |
| 17X | 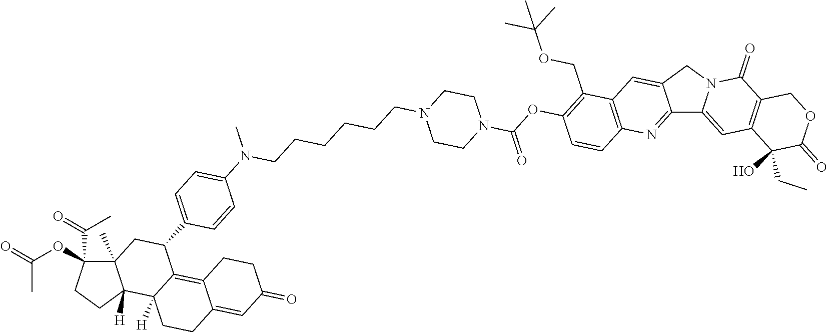 [SMILES: CC[C@@]1(O)C(=O)OCc2c1cc1n(c2=O)Cc2cc3c(COC(C)(C)C)c(OC(=O)N4CCN(CCCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)ccc3nc2-1] |
| 18X | 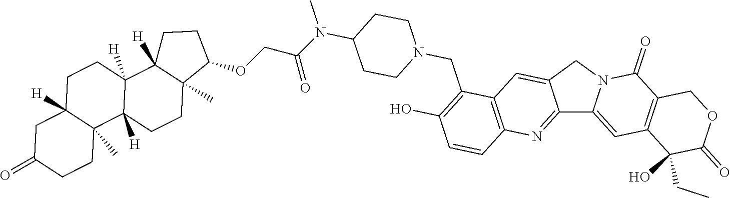 [SMILES: CC[C@@]1(O)C(=O)OCc2c1cc1n(c2=O)Cc2cc3c(CN4CCC(N(C)C(=O)CO[C@H]5CC[C@H]6[C@@H]7CC[C@H]8CC(=O)CC[C@]8(C)[C@H]7CC[C@]56C)CC4)c(O)ccc3nc2-1] |
| 19X | 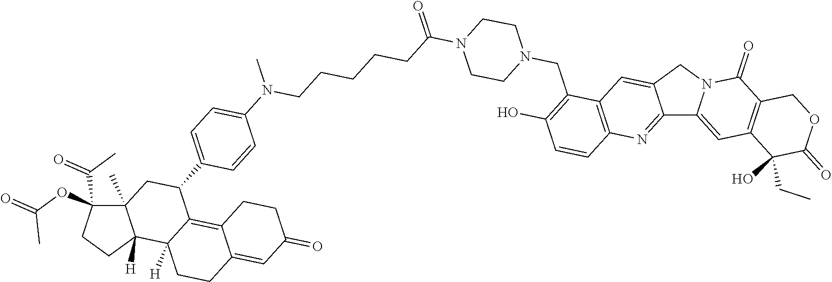 [SMILES: CC[C@@]1(O)C(=O)OCc2c1cc1n(c2=O)Cc2cc3c(CN4CCN(C(=O)CCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)c(O)ccc3nc2-1] |
| 20X | 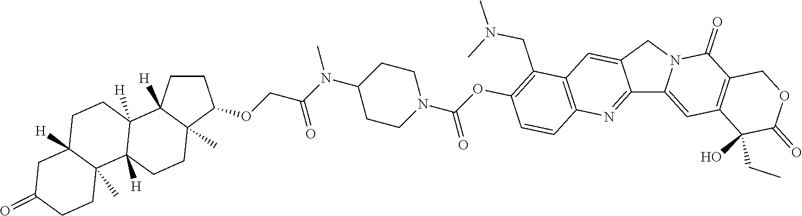 [SMILES: CC[C@@]1(O)C(=O)OCc2c1cc1n(c2=O)Cc2cc3c(CN(C)C)c(OC(=O)N4CCC(N(C)C(=O)CO[C@H]5CC[C@H]6[C@@H]7CC[C@H]8CC(=O)CC[C@]8(C)[C@H]7CC[C@]56C)CC4)ccc3nc2-1] |
| 21X | 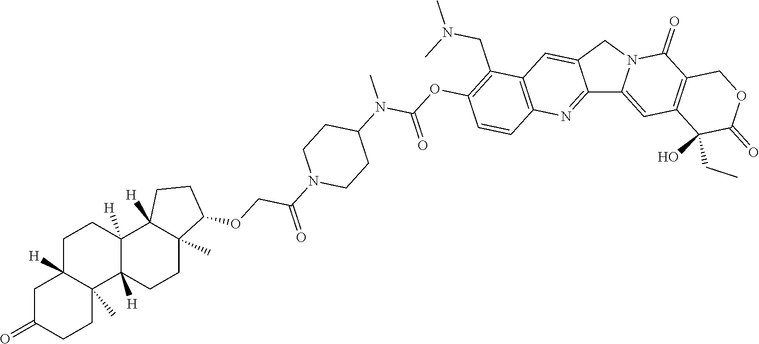 [SMILES: CC[C@@]1(O)C(=O)OCc2c1cc1n(c2=O)Cc2cc3c(CN(C)C)c(OC(=O)N(C)C4CCN(C(=O)CO[C@H]5CC[C@H]6[C@@H]7CC[C@H]8CC(=O)CC[C@]8(C)[C@H]7CC[C@]56C)CC4)ccc3nc2-1] |
| 22X | 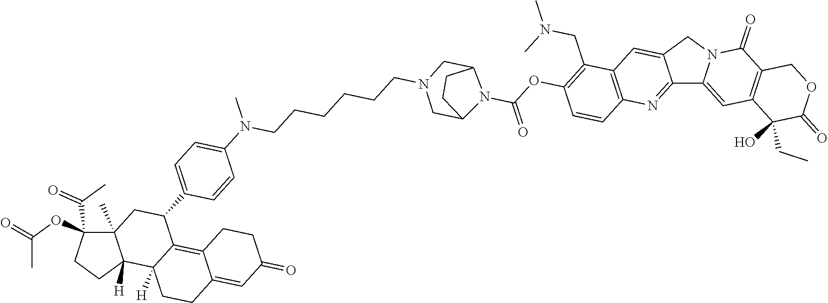 [SMILES: CC[C@@]1(O)C(=O)OCc2c1cc1n(c2=O)Cc2cc3c(CN(C)C)c(OC(=O)N4C5CCC4CN(CCCCCCN(C)c4ccc([C@H]6C[C@@]7(C)[C@@H](CC[C@]7(OC(C)=O)C(C)=O)[C@@H]7CCC8=CC(=O)CCC8=C76)cc4)C5)ccc3nc2-1] |
| 23X | 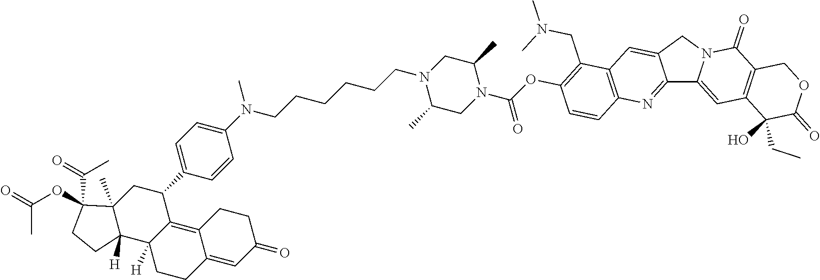 [SMILES: CC[C@@]1(O)C(=O)OCc2c1cc1n(c2=O)Cc2cc3c(CN(C)C)c(OC(=O)N4C[C@H](C)N(CCCCCCN(C)c5ccc([C@H]6C[C@@]7(C)[C@@H](CC[C@]7(OC(C)=O)C(C)=O)[C@@H]7CCC8=CC(=O)CCC8=C76)cc5)C[C@H]4C)ccc3nc2-1] |
| 24X | 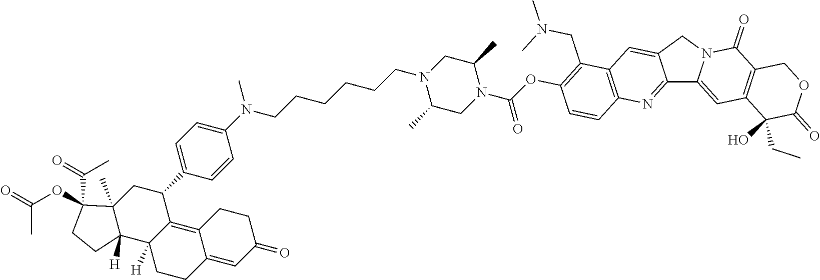 [SMILES: CC[C@@]1(O)C(=O)OCc2c1cc1n(c2=O)Cc2cc3c(CN(C)C)c(OC(=O)N4C[C@H](C)N(CCCCCCN(C)c5ccc([C@H]6C[C@@]7(C)[C@@H](CC[C@]7(OC(C)=O)C(C)=O)[C@@H]7CCC8=CC(=O)CCC8=C76)cc5)C[C@H]4C)ccc3nc2-1] |
| 25X | 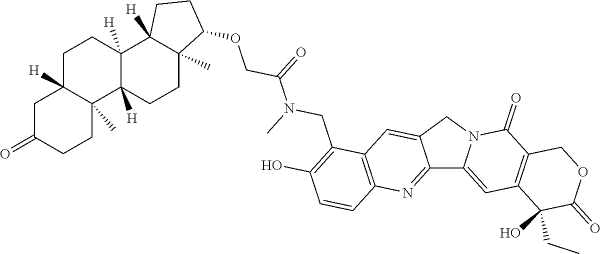 [SMILES: CC[C@@]1(O)C(=O)OCc2c1cc1n(c2=O)Cc2cc3c(CN(C)C(=O)CO[C@H]4CC[C@H]5[C@@H]6CC[C@H]7CC(=O)CC[C@]7(C)[C@H]6CC[C@]45C)c(O)ccc3nc2-1] |
| 26X | 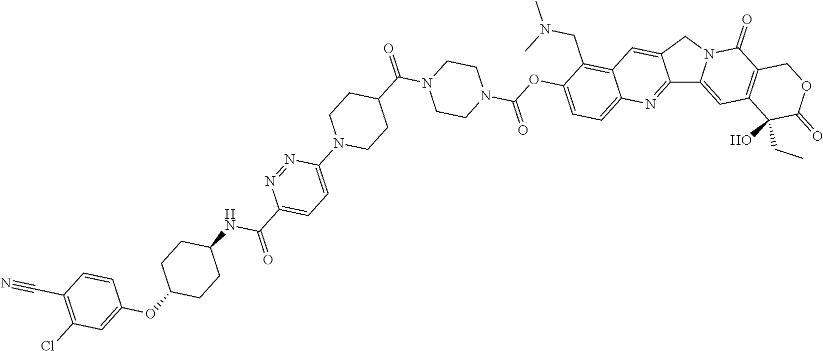 [SMILES: CC[C@@]1(O)C(=O)OCc2c1cc1n(c2=O)Cc2cc3c(CN(C)C)c(OC(=O)N4CCN(C(=O)C5CCN(c6ccc(C(=O)N[C@H]7CC[C@H](Oc8ccc(C#N)c(Cl)c8)CC7)nn6)CC5)CC4)ccc3nc2-1] |
| 27X | 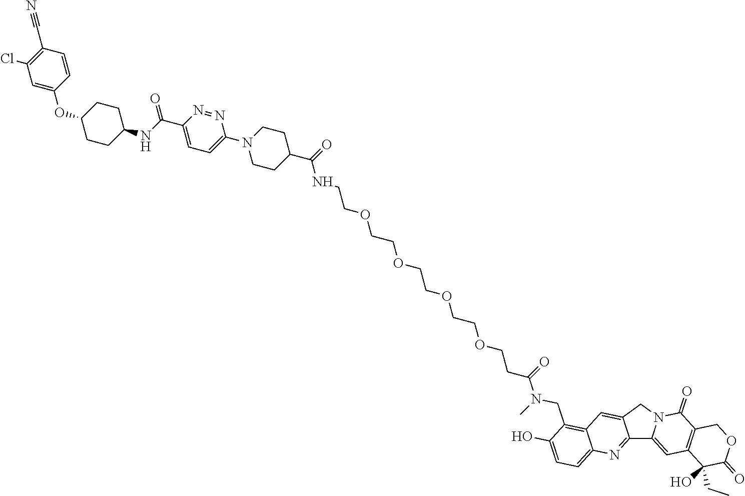 [SMILES: CC[C@@]1(O)C(=O)OCc2c1cc1n(c2=O)Cc2cc3c(CN(C)C(=O)CCOCCOCCOCCOCCNC(=O)C4CCN(c5ccc(C(=O)N[C@H]6CC[C@H](Oc7ccc(C#N)c(Cl)c7)CC6)nn5)CC4)c(O)ccc3nc2-1] |
| 28X | 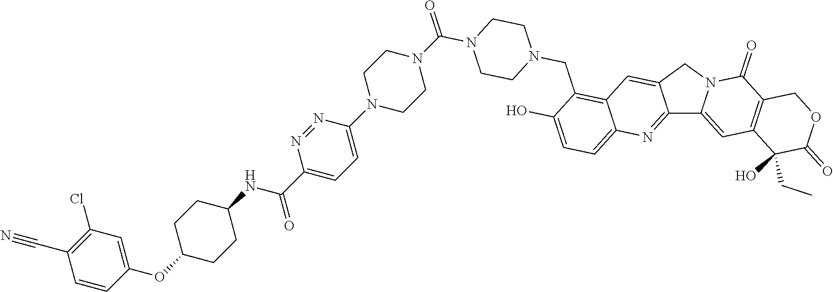 [SMILES: CC[C@@]1(O)C(=O)OCc2c1cc1n(c2=O)Cc2cc3c(CN4CCN(C(=O)N5CCN(c6ccc(C(=O)N[C@H]7CC[C@H](Oc8ccc(C#N)c(Cl)c8)CC7)nn6)CC5)CC4)c(O)ccc3nc2-1] |
| 29X | 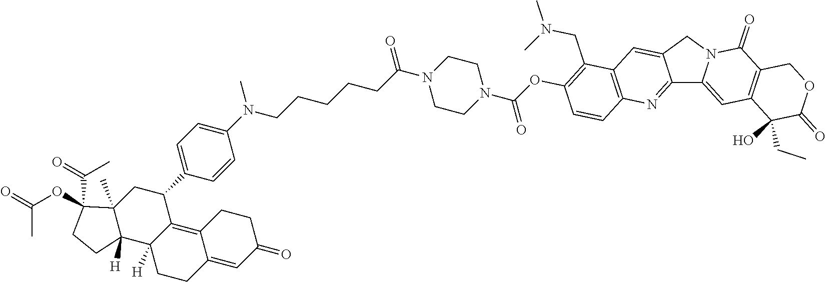 [SMILES: CC[C@@]1(O)C(=O)OCc2c1cc1n(c2=O)Cc2cc3c(CN(C)C)c(OC(=O)N4CCN(C(=O)CCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)ccc3nc2-1] |
| 30X | 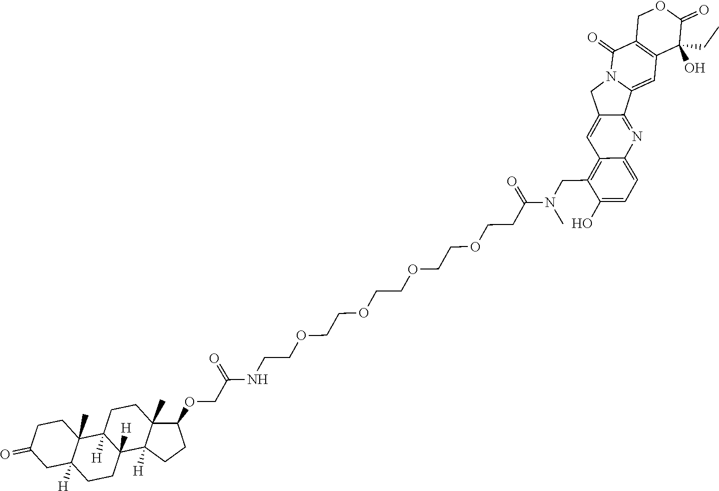 |

In certain embodiments, R¹ is hydrogen.

In certain embodiments, R¹ is C₁₋₁₂ alkyl, which is optionally substituted with one or more R¹⁰.

In certain embodiments, R¹ is —Si(R¹⁵)₃, which is optionally substituted with one or more R¹⁰.

In certain embodiments, R¹ is —C═NOR¹⁵, which is optionally substituted with one or more R¹⁰.

In certain embodiments, R¹ is ethyl.

In certain embodiments, R¹ is

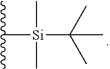 [SMILES: *[Si](C)(C)C(C)(C)C]

In certain embodiments, R¹ is

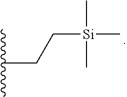 [SMILES: *CC[Si](C)(C)C]

In certain embodiments, R¹ is

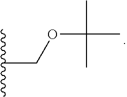 [SMILES: *COC(C)(C)C]

In certain embodiments, R¹ is

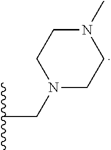 [SMILES: *CN1CCN(C)CC1]

In certain embodiments, R¹ is

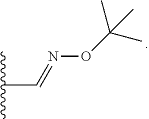 [SMILES: */C=N/OC(C)(C)C]

In certain embodiments, R² is hydrogen.

In certain embodiments, R² is C₁₋₁₂ alkyl optionally substituted with one or more R¹⁰.

In certain embodiments, R² is

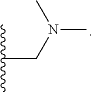 [SMILES: *CN(C)C]

In certain embodiments, R² is nitro.

In certain embodiments, R² is

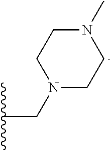 [SMILES: *CN1CCN(C)CC1]

In certain embodiments, R¹ and R² are taken together with the atoms to which they are attached to form a C₃₋₁₂ cycloalkyl, which is optionally substituted with one or more R¹⁰.

In certain embodiments, R¹ and R² are taken together with the atoms to which they are attached to form

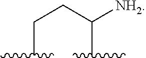 [SMILES: *CCC(*)N]

In certain embodiments, R³ is —OR¹⁵, which is optionally substituted with one or more R¹⁰.

In certain embodiments, R³ is C₁₋₁₂ alkyl, which is optionally substituted with one or more R¹⁰.

In certain embodiments, R³ is —OC(═O)NR¹⁵R¹⁶, which is optionally substituted with one or more R¹⁰.

In certain embodiments, R³ is —OH.

In certain embodiments, R³ is methyl.

In certain embodiments, R³ is

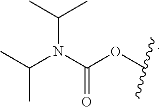 [SMILES: *OC(=O)N(C(C)C)C(C)C]

In certain embodiments, R³ is

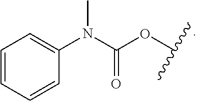 [SMILES: *OC(=O)N(C)c1ccccc1]

In certain embodiments, R³ is

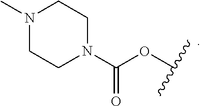 [SMILES: *OC(=O)N1CCN(C)CC1]

In certain embodiments, R³ is

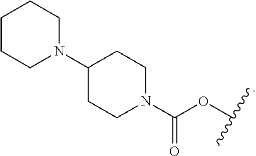 [SMILES: *OC(=O)N1CCC(N2CCCCC2)CC1]

In certain embodiments, R³ is

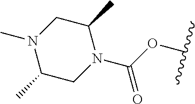 [SMILES: *OC(=O)N1C[C@H](C)N(C)C[C@H]1C]

In certain embodiments, R³ is methoxy.

In certain embodiments, R³ is hydrogen.

In certain embodiments, R⁴ is hydrogen.

In certain embodiments, R⁴ is halo.

In certain embodiments, R³ and R⁴ are taken together with the atoms to which they are attached to form a 5- to 12-membered heterocyclyl, which is optionally substituted with one or more R¹⁰.

In certain embodiments, R³ and R⁴ are taken together with the atoms to which they are attached to form

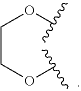 [SMILES: *OCCO*]

In certain embodiments, R⁵ is hydrogen.

In certain embodiments, R⁵ is —C(═O)R¹⁵, which is optionally substituted with one or more R¹⁰.

In certain embodiments, R⁵ is

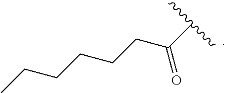 [SMILES: *C(=O)CCCCCC]

In certain embodiments, R⁵ is

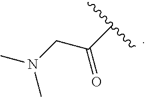 [SMILES: *C(=O)CN(C)C]

In certain embodiments, A¹ is derived from:

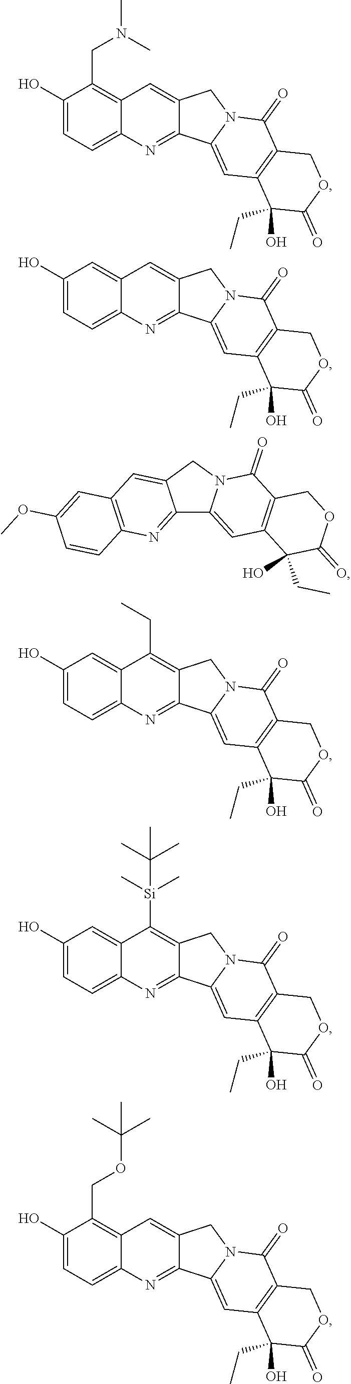

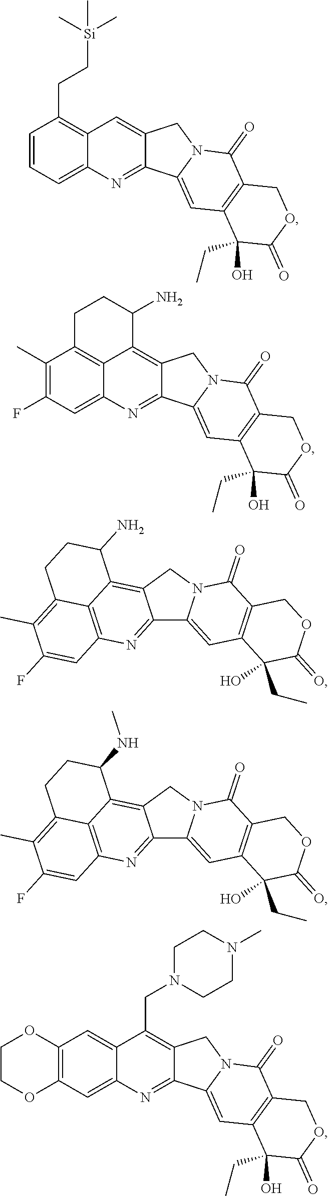

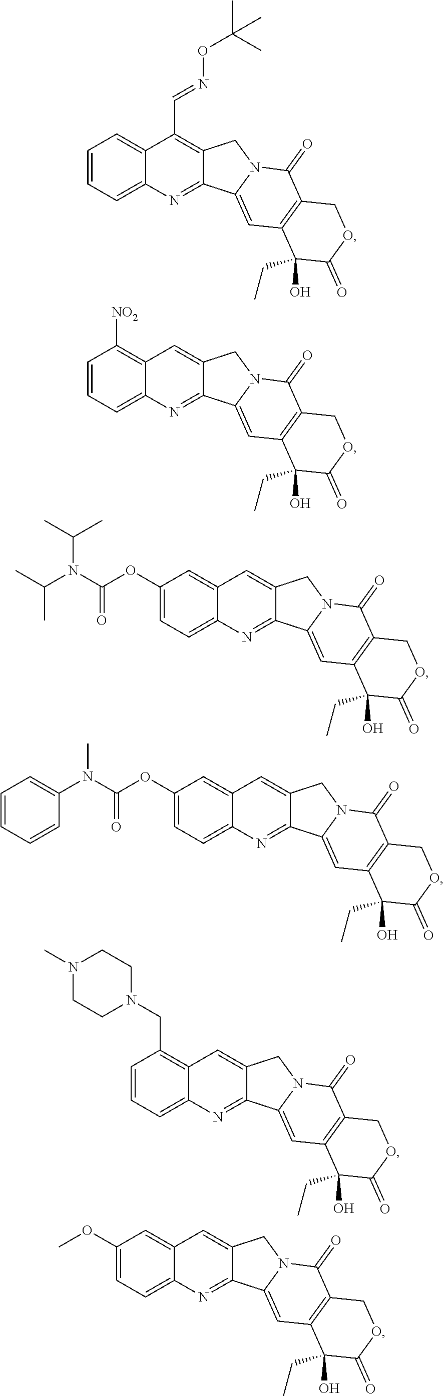

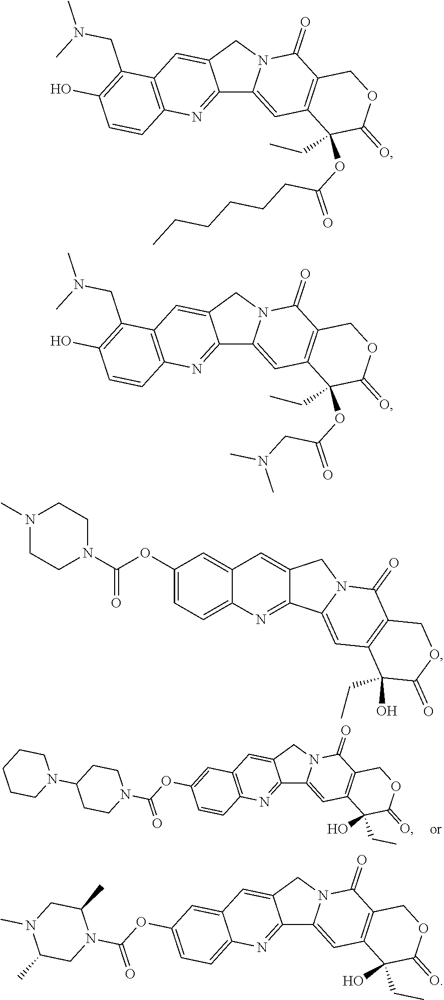

In certain embodiments, A¹ is derived from:

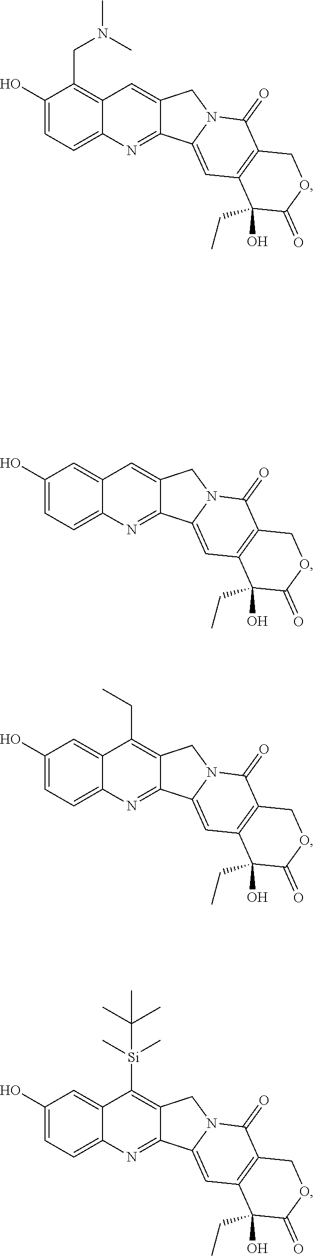 [SMILES: CC[C@@]1(O)C(=O)OCc2c1cc1n(c2=O)Cc2cc3c(CN(C)C)c(O)ccc3nc2-1 ; CC[C@@]1(O)C(=O)OCc2c1cc1n(c2=O)Cc2cc3cc(O)ccc3nc2-1 ; CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2c-1nc1ccc(O)cc1c2[Si](C)(C)C(C)(C)C]

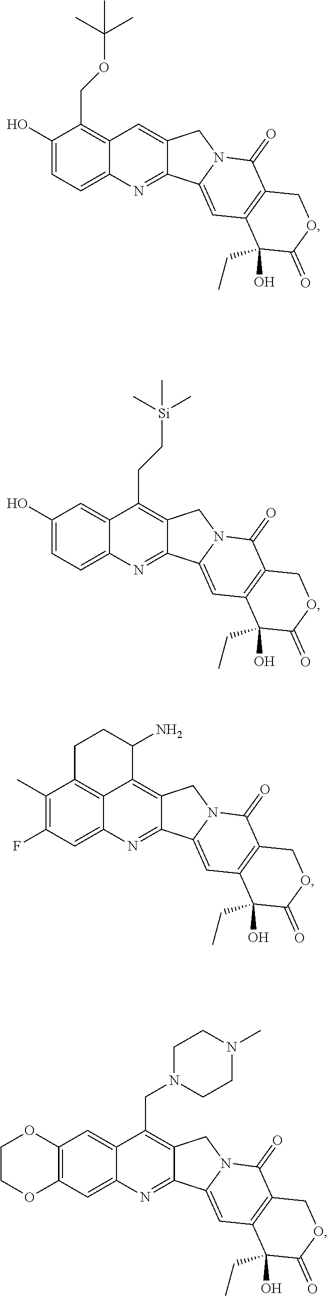 [SMILES: CC[C@@]1(O)C(=O)OCc2c1cc1n(c2=O)Cc2cc3c(COC(C)(C)C)c(O)ccc3nc2-1 ; CC[C@@]1(O)C(=O)OCc2c1cc1n(c2=O)Cc2c-1nc1ccc(O)cc1c2CC[Si](C)(C)C ; CC[C@@]1(O)C(=O)OCc2c1cc1n(c2=O)Cc2c-1nc1cc(F)c(C)c3c1c2C(N)CC3 ; CC[C@@]1(O)C(=O)OCc2c1cc1n(c2=O)Cc2c-1nc1cc3c(cc1c2CN1CCN(C)CC1)OCCO3]

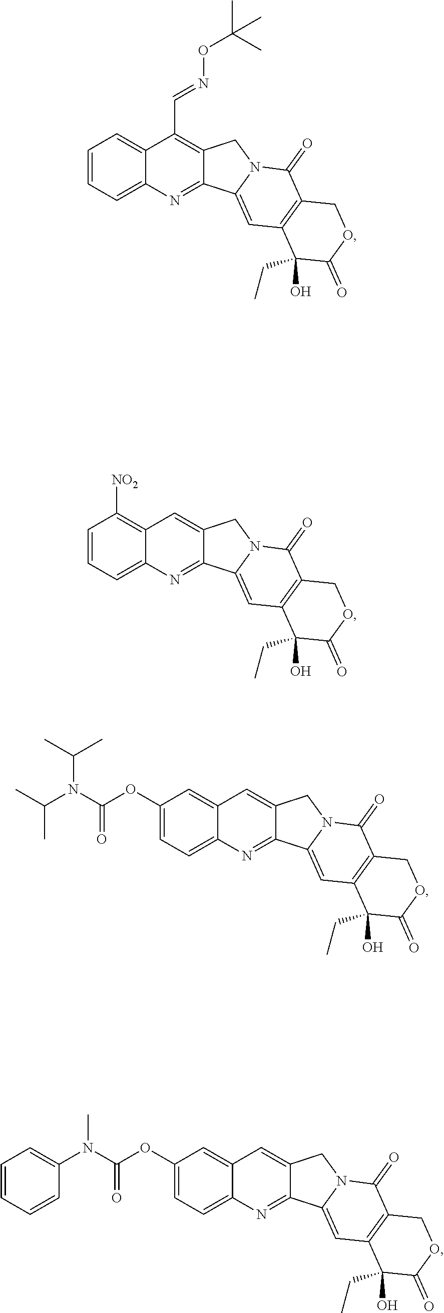 [SMILES: CC[C@@]1(O)C(=O)OCc2c1cc1n(c2=O)Cc2c-1nc1ccccc1c2/C=N/OC(C)(C)C ; CC[C@@]1(O)C(=O)OCc2c1cc1n(c2=O)Cc2cc3c([N+](=O)[O-])cccc3nc2-1 ; CC[C@@]1(O)C(=O)OCc2c1cc1n(c2=O)Cc2cc3cc(OC(=O)N(C(C)C)C(C)C)ccc3nc2-1 ; CC[C@@]1(O)C(=O)OCc2c1cc1n(c2=O)Cc2cc3cc(OC(=O)N(C)c4ccccc4)ccc3nc2-1]

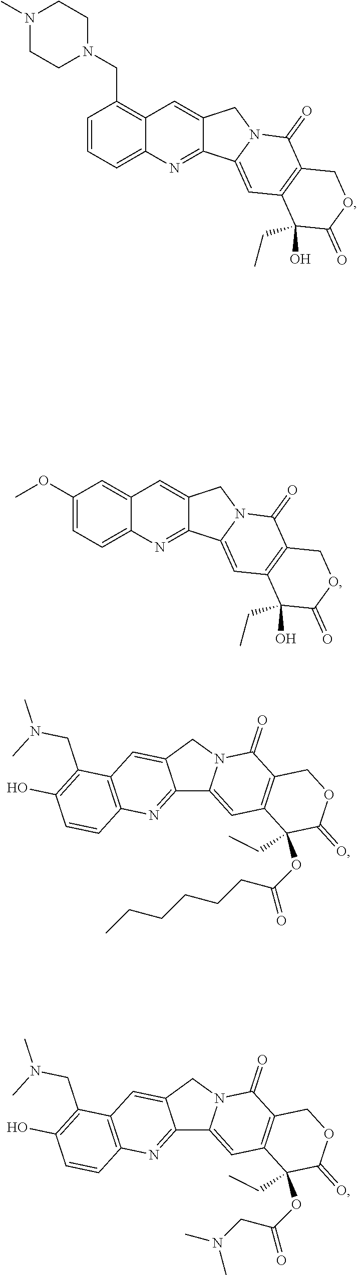 [SMILES: CC[C@@]1(O)C(=O)OCc2c1cc1n(c2=O)Cc2cc3c(CN4CCN(C)CC4)cccc3nc2-1 ; CC[C@@]1(O)C(=O)OCc2c1cc1n(c2=O)Cc2cc3cc(OC)ccc3nc2-1 ; CCCCCCC(=O)O[C@]1(CC)C(=O)OCc2c1cc1n(c2=O)Cc2cc3c(CN(C)C)c(O)ccc3nc2-1 ; CC[C@@]1(OC(=O)CN(C)C)C(=O)OCc2c1cc1n(c2=O)Cc2cc3c(CN(C)C)c(O)ccc3nc2-1]

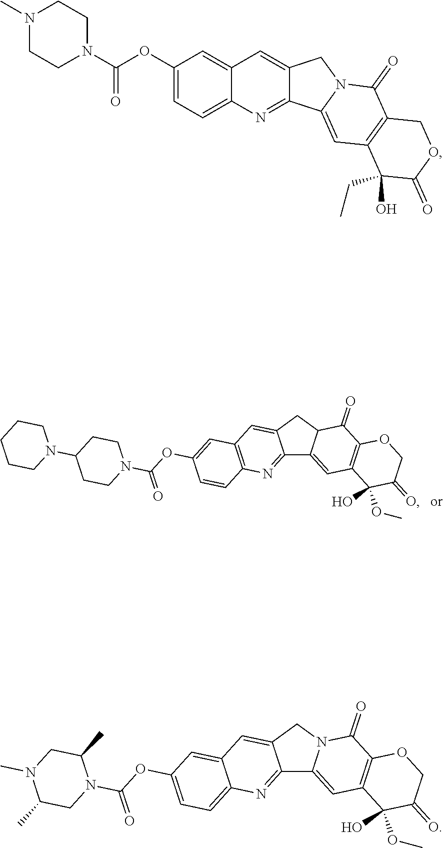 [SMILES: CC[C@@]1(O)C(=O)OCc2c1cc1n(c2=O)Cc2cc3cc(OC(=O)N4CCN(C)CC4)ccc3nc2-1 ; CO[C@@]1(O)C(=O)COC2=C1C=C1c3nc4ccc(OC(=O)N5CCC(N6CCCCC6)CC5)cc4cc3CC1C2=O ; "or" ; CO[C@@]1(O)C(=O)COc2c1cc1n(c2=O)Cc2cc3cc(OC(=O)N4C[C@H](C)N(C)C[C@H]4C)ccc3nc2-1]

In certain embodiments, a hydrogen atom of Formula IA is replaced by a direct covalent bond to L¹. In certain embodiments, a methyl of Formula IA is replaced by a direct covalent bond to L¹. In certain embodiments, a hydroxyl of Formula IA is replaced by a direct covalent bond to L¹. In certain embodiments, L¹ is linked to a nitrogen atom of A¹. In certain embodiments, n L¹ is linked to an oxygen atom of A¹.

In certain embodiments, A¹ is:

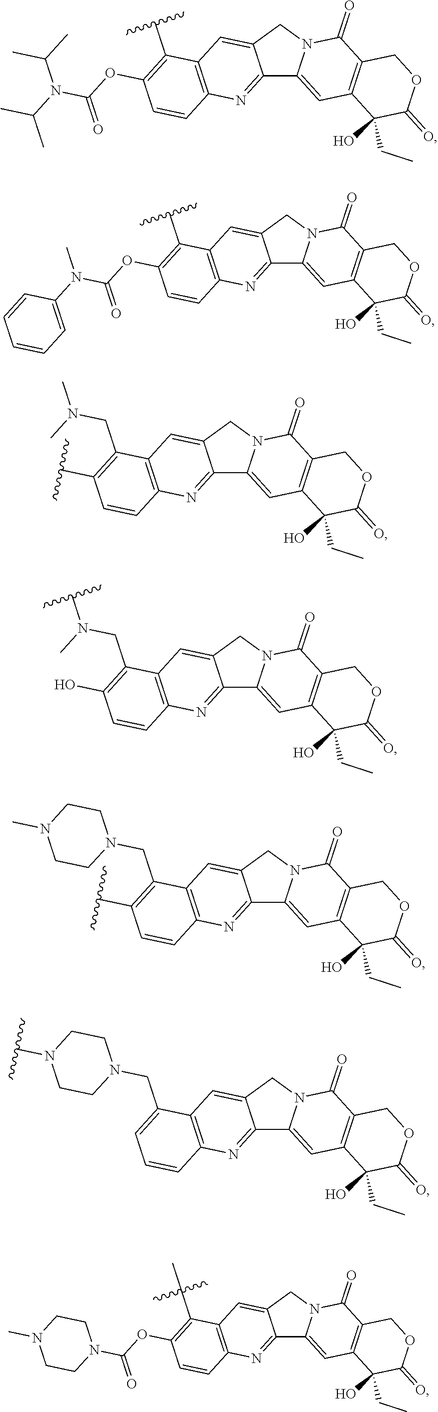

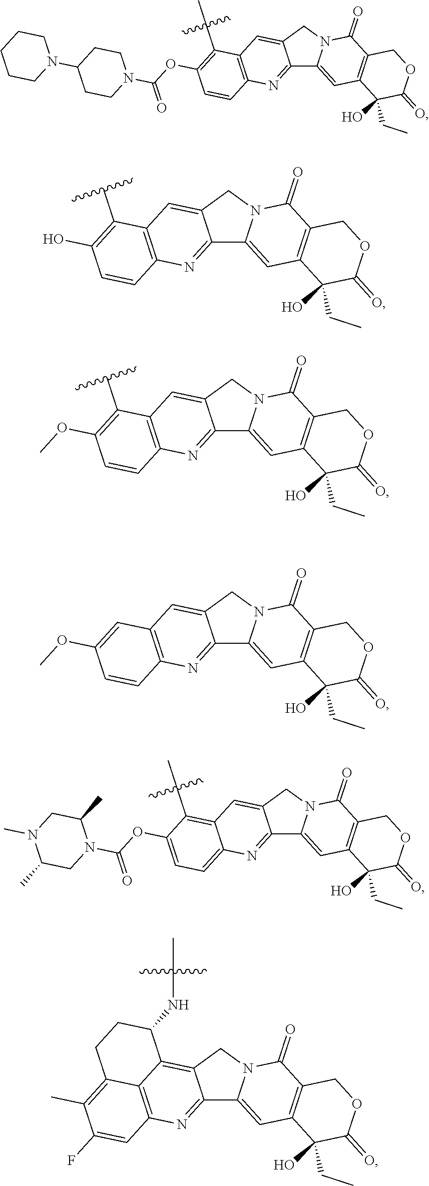 [SMILES: *c1c(OC(=O)N2CCC(N3CCCCC3)CC2)ccc2nc3c(cc12)Cn1c-3cc2c(c1=O)COC(=O)[C@]2(O)CC ; *c1c(O)ccc2nc3c(cc12)Cn1c-3cc2c(c1=O)COC(=O)[C@]2(O)CC ; *c1c(OC)ccc2nc3c(cc12)Cn1c-3cc2c(c1=O)COC(=O)[C@]2(O)CC ; CC[C@@]1(O)C(=O)OCc2c1cc1n(c2=O)Cc2cc3cc(OC)ccc3nc2-1 ; *c1c(OC(=O)N2C[C@H](C)N(C)C[C@H]2C)ccc2nc3c(cc12)Cn1c-3cc2c(c1=O)COC(=O)[C@]2(O)CC ; *N[C@H]1CCc2c(C)c(F)cc3nc4c(c1c23)Cn1c-4cc2c(c1=O)COC(=O)[C@]2(O)CC]

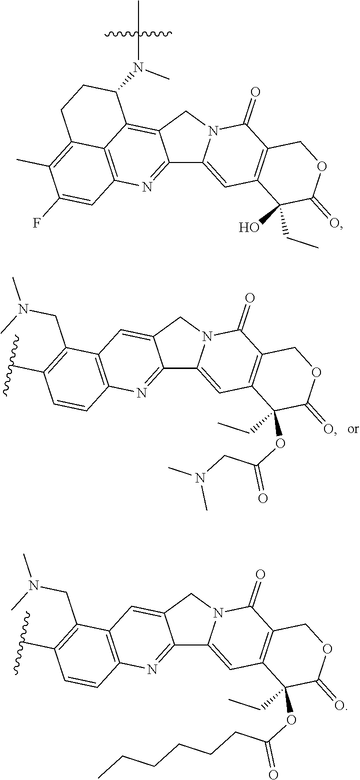 [SMILES: *N(C)[C@H]1CCc2c(C)c(F)cc3nc4c(c1c23)Cn1c-4cc2c(c1=O)COC(=O)[C@]2(O)CC ; *c1ccc2nc3c(cc2c1CN(C)C)Cn1c-3cc2c(c1=O)COC(=O)[C@@]2(CC)OC(=O)CN(C)C ; *c1ccc2nc3c(cc2c1CN(C)C)Cn1c-3cc2c(c1=O)COC(=O)[C@@]2(CC)OC(=O)CCCCCC]

In certain embodiments, A¹ is:

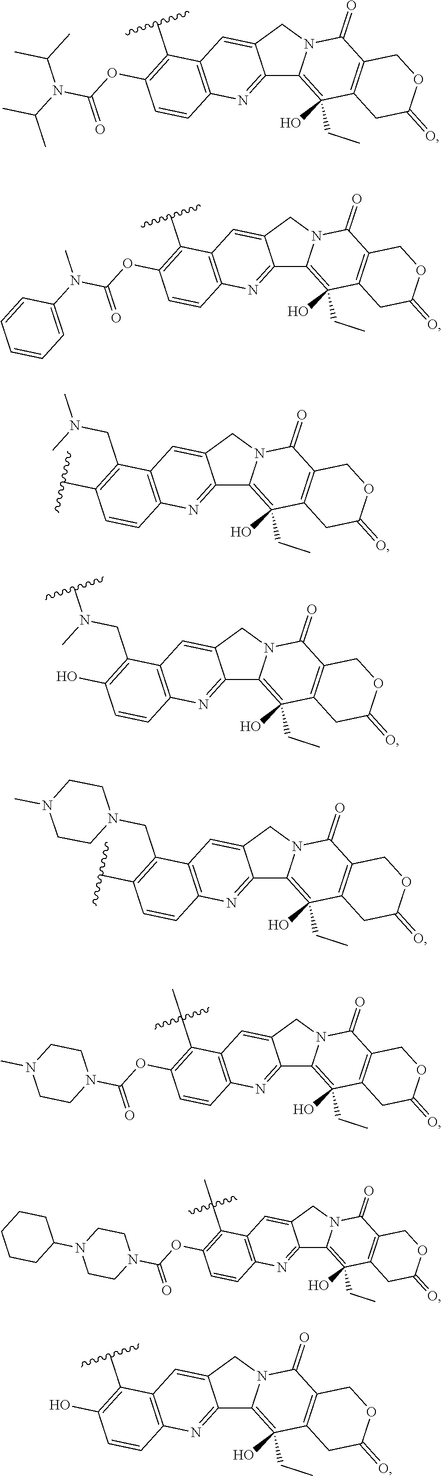

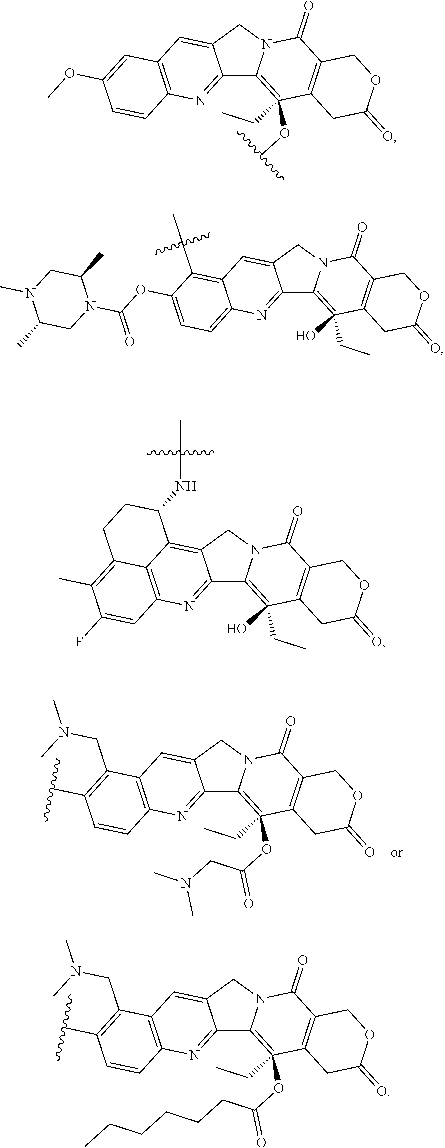

In certain embodiments, any of the compounds disclosed herein (e.g., a compound of Formula I) comprises a topoisomerase inhibitor analog, which even after modification to arrive at the compounds described herein, exhibit a biological activity which is comparable to that observed in the original, unmodified topoisomerase inhibitor. In certain embodiments, the topoisomerase inhibitor analogs maintain the ability to inhibit a topoisomerase. In certain embodiments, the topoisomerase inhibitor analogs exhibit a binding activity which is at least about 98%, about 95%, about 90%, about 85%, about 80%, about 75%, about 70%, about 65%, about 60%, about 55%, or about 50% of that observed in the original, unmodified topoisomerase inhibitor.

### Nuclear Payloads

In certain embodiments, the nuclear payload (i.e., A¹) in the compounds described herein, is a topoisomerase inhibitor. As used herein, the term “topoisomerase inhibitor” refers to a chemical compound or moiety that blocks the action of a topoisomerase (or DNA topoisomerase), which are enzymes that participate in the overwinding or underwinding of DNA.

In certain embodiments, the nuclear payload (i.e., A¹) of the compounds described herein, is derived from camptothecin (CPT). As such, in certain embodiments, the nuclear payload (i.e., A¹) of the compounds described herein, is a camptothecin (CPT) analog. In certain embodiments, the nuclear payload (i.e., A¹) of the compounds described herein, is derived from topotecan, irinotecan (CPT-11), silatecan (DB-67, AR-67), cositecan (BNP-1350), exatecan, lurtotecan, gimatecan (ST1481), belotecan (CKD-602), or rubitecan, or an analog thereof.

In certain embodiments, the term “derived from” or “analog” as used in reference to a nuclear payload (i.e., A¹), means that at most, one non-hydrogen atom of an original, unmodified nuclear payload (i.e., a known topoisomerase inhibitor) is replaced by a covalent bond to the nuclear receptor-targeting epitope, optionally via a linking moiety. Exemplary non-hydrogen atoms include, but are not limited to, —CH₃, —OH, ═O, and —NH₂. In certain embodiments, the term “derived from” as used in reference to a nuclear payload (i.e., A¹), means that one or more atoms (e.g., hydrogen, methyl, or hydroxy) of an original, unmodified nuclear payload (i.e., a topoisomerase inhibitor) is replaced by a direct covalent bond to L¹. Exemplary non-hydrogen atoms include, but are not limited to, —CH₃, —OCH₃, —OH, ═O, —NH₂, —N(CH₃)₂, and the like. In certain embodiments, one hydrogen atom bound to a heteroatom (e.g., N, O, or S) of an original, unmodified nuclear payload (i.e., a known topoisomerase inhibitor) is replaced by a covalent bond to L¹. In certain embodiments, the term “derived from” means that one or more atoms (e.g., hydrogen, methyl, or hydroxy) is replaced by a direct covalent bond to L¹.

In certain embodiments, one or more atoms one or more atoms (e.g., hydrogen, methyl, hydroxy, amino, etc.) on the nuclear payload (i.e., A¹) as disclosed herein is replaced for attachment to the remainder of the compound (e.g., the moiety -L¹-B¹). In certain embodiments, a hydrogen atom on a nuclear receptor-targeting epitope disclosed herein is replaced for attachment to the remainder of the compound. In certain embodiments, the hydrogen atom is on a heteroatom. In certain embodiments, the hydrogen atom is on a halogen. In certain embodiments, the hydrogen atom is on a nitrogen. In certain embodiments, the hydrogen atom is on an oxygen. In certain embodiments, the hydrogen atom is on a carbon (e.g., methyl group). The analogs are derived from the known nuclear payload described herein (e.g., topoisomerase inhibitor or A¹) and are modified to be conjugated to at least one nuclear hormone receptor-targeting epitope, optionally via a linking moiety. The analogs, even after modification to arrive at the compounds described herein, maintain biological activity, which is comparable to that observed in the original, unmodified topoisomerase inhibitor. In certain embodiments, the compounds exhibit a binding activity or inhibition which is at least about 98%, about 95%, about 90%, about 85%, about 80%, about 75%, about 70%, about 65%, about 60%, about 55%, or about 50%, or about 5-50% of that observed in the original, unmodified topoisomerase inhibitor. In certain embodiments, the compound as described herein exhibits an IC₅₀ of less than about 500 nM, or less than about 400 nM, or less than about 350 nM, or less than about 300 nM, or less than about 200 nM, or less than about 100 nM, or less than about 50 nM.

In certain embodiments, the nuclear payload (i.e., A¹) is derived from:

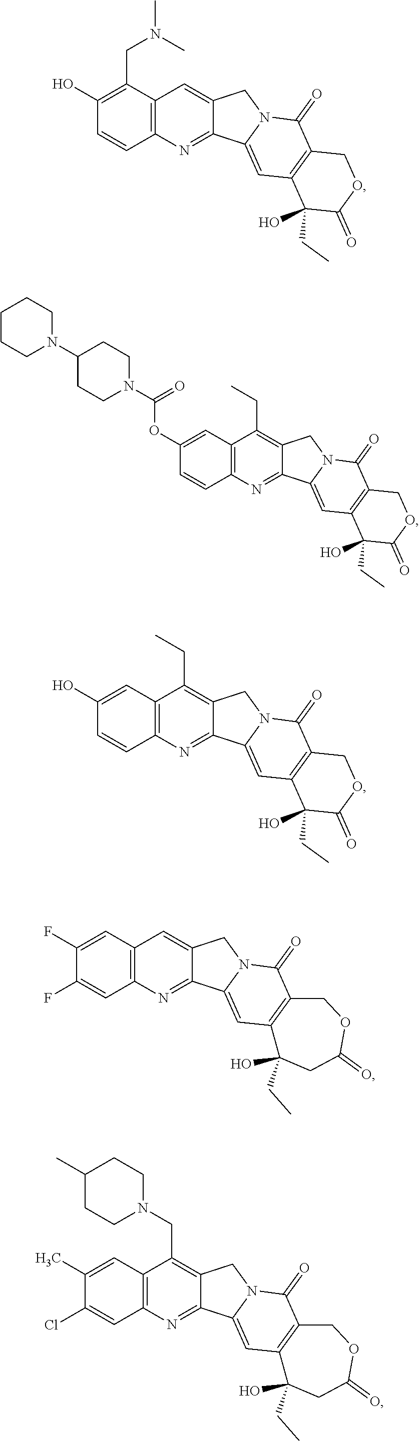 [SMILES: CC[C@@]1(O)C(=O)OCc2c1cc1n(c2=O)Cc2cc3c(CN(C)C)c(O)ccc3nc2-1 ; CCc1c2c(nc3ccc(OC(=O)N4CCC(N5CCCCC5)CC4)cc13)-c1cc3c(c(=O)n1C2)COC(=O)[C@]3(O)CC ; CCc1c2c(nc3ccc(O)cc13)-c1cc3c(c(=O)n1C2)COC(=O)[C@]3(O)CC ; CC[C@@]1(O)CC(=O)OCc2c1cc1n(c2=O)Cc2cc3cc(F)c(F)cc3nc2-1 ; CC[C@@]1(O)CC(=O)OCc2c1cc1n(c2=O)Cc2c-1nc1cc(Cl)c(C)cc1c2CN1CCC(C)CC1]

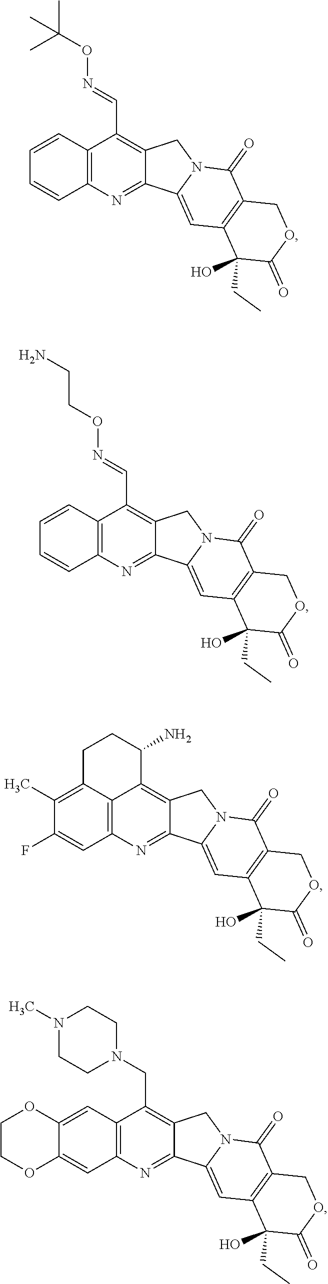 [SMILES: CC[C@@]1(O)C(=O)OCc2c1cc1n(c2=O)Cc2c-1nc1ccccc1c2/C=N/OC(C)(C)C ; CC[C@@]1(O)C(=O)OCc2c1cc1n(c2=O)Cc2c-1nc1ccccc1c2/C=N/OCCN ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc3c(cc1c2CN1CCN(C)CC1)OCCO3]

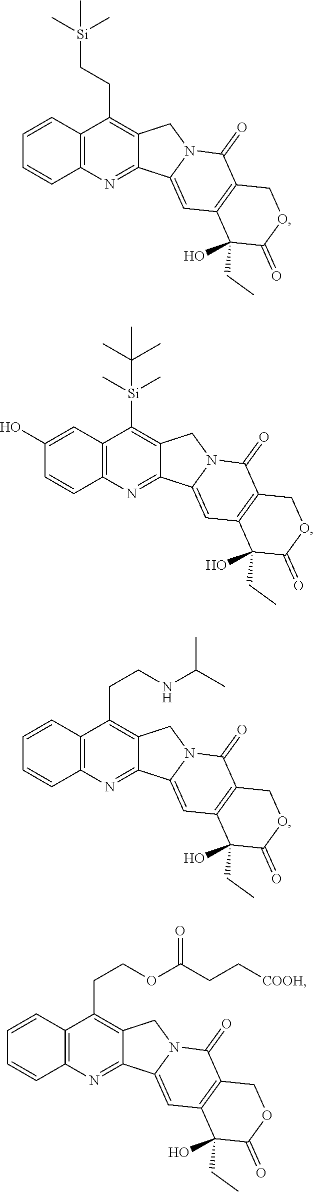 [SMILES: CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccc(O)cc1c2[Si](C)(C)C(C)(C)C ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCOC(=O)CCC(=O)O]

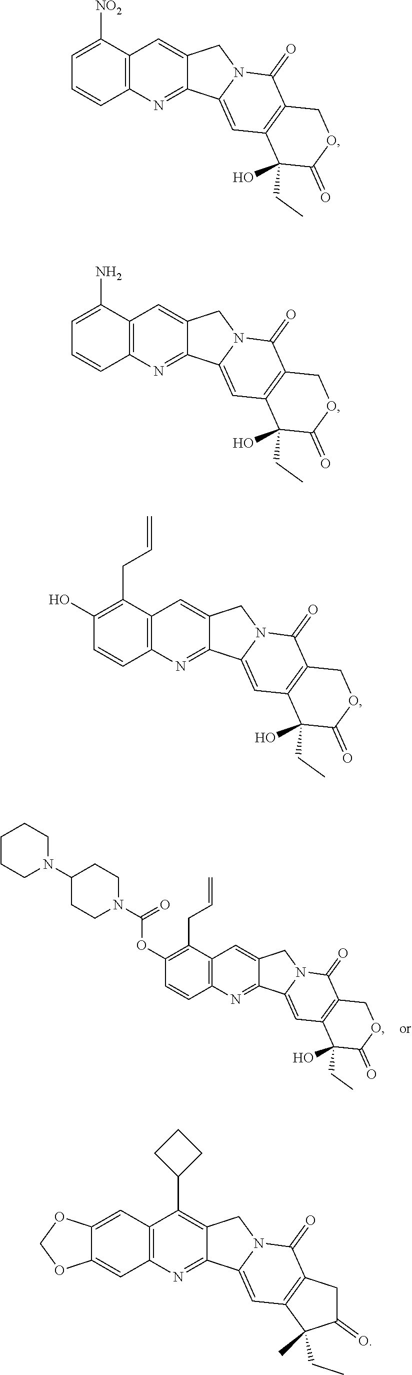 [SMILES: CC[C@@]1(O)C(=O)OCc2c1cc1n(c2=O)Cc2cc3c([N+](=O)[O-])cccc3nc2-1 ; CC[C@@]1(O)C(=O)OCc2c1cc1n(c2=O)Cc2cc3c(N)cccc3nc2-1 ; C=CCc1c(O)ccc2nc3c(cc12)Cn1c-3cc2c(c1=O)COC(=O)[C@]2(O)CC ; C=CCc1c(OC(=O)N2CCC(N3CCCCC3)CC2)ccc2nc3c(cc12)Cn1c-3cc2c(c1=O)COC(=O)[C@]2(O)CC ; CC[C@@]1(C)C(=O)Cc2c1cc1n(c2=O)Cc2c-1nc1cc3c(cc1c2C1CCC1)OCO3]

In certain embodiments, the nuclear payload or A¹ is derived from:

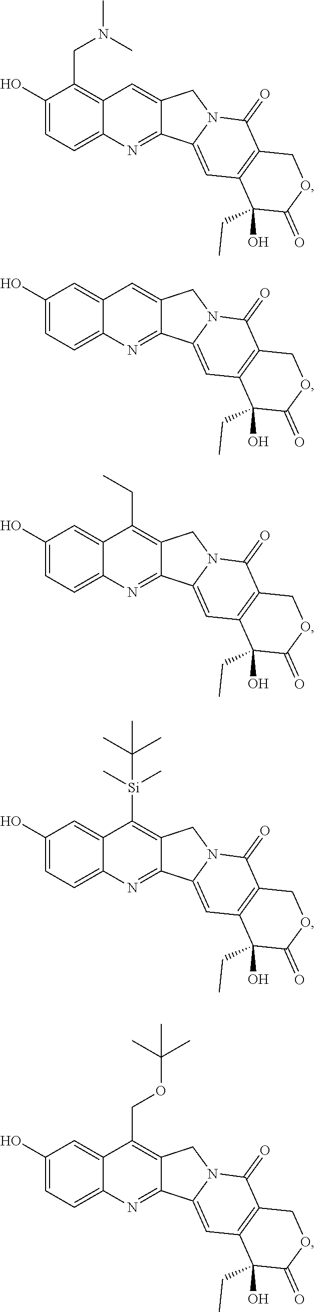

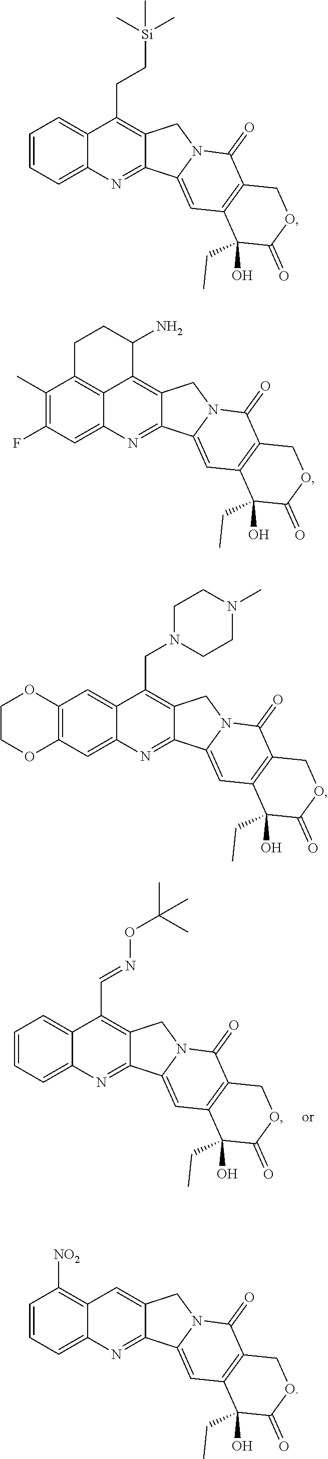

### Nuclear Receptor-Targeting Epitopes

In certain embodiments, B¹ is a nuclear hormone receptor-targeting epitope. In certain embodiments, B¹ binds to an estrogen receptor, glucocorticoid receptor, progesterone receptor, or androgen receptor. In certain embodiments, B¹ binds to estrogen receptor. In certain embodiments, B¹ binds to glucocorticoid receptor. In certain embodiments, B¹ binds to progesterone receptor. In certain embodiments, B¹ binds to androgen receptor. Exemplary estrogen receptor, glucocorticoid receptor, progesterone receptor, or androgen receptor binders are described herein.

In certain embodiments, B¹ is a nuclear steroid receptor-targeting epitope. As used herein, “nuclear receptor-targeting epitope” refers to the portion of the compound described herein (e.g., B¹) which portion is derived from a nuclear targeting agent as disclosed herein and interacts with a ligand-binding domain of the target nuclear receptor, i.e., the portion of the compound which drives a ligand-binding interaction. The nuclear receptor-targeting epitope serves to associate the compound with a target nuclear receptor, e.g. a nuclear steroid receptor, facilitate the localization of compound to nuclear steroid receptor-expressing cells, and translocate the nuclear payload from the cytosol to nucleus, allowing the compound to accumulate in the nucleus. The level of accumulation can be controlled by selecting the appropriate nuclear receptor-targeting epitope. For example, the compounds described herein can accumulate in the nucleus to varying degrees, high in the case of a full agonist (e.g., dihydrotestosterone (DHT)), moderate in the case of a partial agonist (e.g., bicalutamide), and low, in the case of antagonists (e.g., enzalutamide), through nuclear translocation of the nuclear steroid receptor which happens, following epitope binding to the receptor.

The steroid receptor target can be any steroid receptor, including, but not limited to, those which are over-expressed on cancer cells. In certain embodiments, at least one nuclear steroid receptor-targeting epitope is capable of binding to a ligand binding domain of a nuclear steroid receptor, such as a ligand binding domain on an estrogen receptor, glucocorticoid receptor, progesterone receptor or androgen receptor.

Exemplary nuclear steroid receptor-targeting epitopes include those derived from an androgen receptor agonist, an androgen receptor antagonist, a selective androgen-receptor modulator (SARM), an estrogen receptor agonist, an estrogen receptor antagonist, a selective estrogen receptor modulator (SERM), a glucocorticoid receptor antagonist, a glucocorticoid receptor agonist, a selective glucocorticoid receptor modulator (SGRM), a progesterone receptor antagonist, a progesterone receptor agonist, a selective progesterone receptor modulator (SPRM), or a combination thereof.

The nuclear steroid receptor-targeting epitopes are typically capable of binding to a nuclear steroid receptor with an IC₅₀ of less than about 500 nM, or less than about 400 nM, or less than about 300 nM, or less than about 200 nM, or less than about 100 nM, or with an EC₅₀ of less than about 1 μM, or less than about 900 nM, or less than about 800 nM, or less than about 700 nM, or less than about 600 nM, or less than about 500 nM, or less than about 400 nM, or less than about 3400 nM, or less than about 200 nM, or less than about 100 nM.

In certain embodiments, the nuclear hormone receptor binding affinity of a compound of this invention can be defined according to its affinity relative to a reference nuclear hormone receptor binding compound. For example, some compounds of this invention can bind to the estrogen receptor. In some instances, a compound disclosed herein binds the human estrogen receptor with an affinity of at least 0.1%, 1%, 5%, 10%, 20%, 30%, 40%, 50%, 60%, 70%, 80%, 90%, or 100% of that of 17b-estradiol.

By way of additional examples, some compounds of this invention can bind to the human androgen receptor. In some instances, a compound disclosed herein binds the androgen receptor with an affinity of at least 0.1%, 1%, 5%, 10%, 20%, 30%, 40%, 50%, 60%, 70%, 80%, 90%, or 100% of that of dihydrotestosterone (DHT).

By way of additional examples, some compounds of this invention can bind to the human progestin receptor. In some instances, a compound disclosed herein binds the progestin receptor with an affinity of at least 0.1%, 1%, 5%, 10%, 20%, 30%, 40%, 50%, 60%, 70%, 80%, 90%, or 100% of that of progesterone.

By way of additional examples, some compounds of this invention can bind to the human glucocorticoid receptor. In some instances, a compound disclosed herein binds the glucocorticoid receptor with an affinity of at least 0.1%, 1%, 5%, 10%, 20%, 30%, 40%, 50%, 60%, 70%, 80%, 90%, or 100% of that of cortisone.

In certain embodiments, the nuclear steroid receptor-targeting epitope (e.g., B¹) is an agonist at the androgen receptor. In certain embodiments, the nuclear steroid receptor-targeting epitope is an antagonist at the androgen receptor.

In certain embodiments, the nuclear steroid receptor-targeting epitope (e.g., B¹) is steroidal (or is derived from a steroidal compound) (e.g., dihydrotestosterone). In certain embodiments, the nuclear steroid receptor-targeting epitope is non-steroidal (or is derived from a non-steroidal compound) (e.g., enzalutamide, apalutamide, AZD9496 and bicalutamide).

The analogs are derived from the known nuclear steroid receptor-targeting epitope described herein (e.g., B¹) and are modified to be conjugated to at least one nuclear steroid payload, optionally via a linking moiety. The analogs, even after modification to arrive at the compounds described herein, maintain biological activity, which is comparable to that observed in the original, unmodified nuclear steroid receptor-targeting epitope. In certain embodiments, the compounds exhibit a binding activity or inhibition which is at least about 98%, about 95%, about 90%, about 85%, about 80%, about 75%, about 70%, about 65%, about 60%, about 55%, or about 50%, or about 5-50% of that observed in the original, unmodified nuclear steroid receptor-targeting epitope.

In certain embodiments, the analogs are derived from a known nuclear receptor-targeting epitope (e.g., B¹), such as a known nuclear steroid receptor-targeting epitope. In certain embodiments, B¹ binds to an estrogen receptor, glucocorticoid receptor, progesterone receptor, or androgen receptor. In certain embodiments, the term “derived from” as used in reference to a nuclear receptor-targeting epitope, means that at most, one non-hydrogen atom of an original, unmodified nuclear receptor-targeting compound (i.e., a known nuclear steroid receptor-targeting compound) is replaced by a covalent bond to the nuclear payload, optionally via a linking moiety. Exemplary non-hydrogen atoms include, but are not limited to, —CH₃, —OH, ═O, and —NH₂. In certain embodiments, the term “derived from” as used in reference to a nuclear receptor-targeting epitope, means that at most, one non-hydrogen atom of an original, unmodified nuclear receptor-targeting compound (i.e., a known nuclear steroid receptor-targeting compound) is replaced by a covalent bond to the nuclear payload, optionally via a linking moiety. In certain embodiments, one hydrogen atom bound to a heteroatom (e.g., N, O, or S) of the original, unmodified nuclear receptor-targeting compound (i.e., a known nuclear steroid receptor-targeting compound) is replaced by a covalent bond to the nuclear payload, optionally via a linking moiety. In certain embodiments, the term “derived from” means that one or more atoms (e.g., hydrogen, methyl, or hydroxy) is replaced by a direct covalent bond to L¹.

In certain embodiments, the nuclear steroid receptor-targeting epitope (e.g., B¹) is an androgen receptor-targeting epitope. As used herein, the term “androgen receptor-targeting epitope” is intended to refer to the portion of the compound which binds to the androgen receptor and can functionally be an androgen receptor agonist or androgen receptor antagonist (including partial androgen receptor agonists or partial androgen receptor antagonists) and in some embodiments, is capable of binding to the receptor and the ligand receptor complex shuttling from the cytoplasm into the nucleus of a cell. The “androgen receptor” (AR), also known as NR3C4 (nuclear receptor subfamily 3, group C, member 4), is a type of nuclear receptor that, when activated by binding an androgen receptor binder (e.g., an androgenic hormone such as testosterone, or dihydrotestosterone) in the cytoplasm, is capable of translocating the androgenic hormone into the nucleus.

In certain embodiments, a single atom on the nuclear receptor-targeting epitope (B¹) as disclosed herein is replaced for attachment to the remainder of the compound (e.g., the moiety -L¹-B¹). In certain embodiments, a halogen atom on a nuclear receptor-targeting epitope disclosed herein is replaced for attachment to the remainder of the compound. In certain embodiments, a hydrogen atom on a nuclear receptor-targeting epitope disclosed herein is replaced for attachment to the remainder of the compound. In certain embodiments, the hydrogen atom is on a heteroatom. In certain embodiments, the hydrogen atom is on a nitrogen. In certain embodiments, the hydrogen atom is on an oxygen. In certain embodiments, the hydrogen atom is on a carbon.

In certain embodiments, B¹ is of Formula IIA:

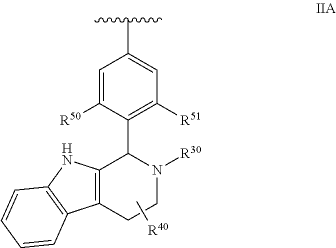

wherein:
the wavy bond represents the point of connection to L¹;
R³⁰ is hydrogen, C₁₋₁₂ alkyl, C₁₋₁₂ haloalkyl, C₂₋₁₂ alkenyl, C₂₋₁₂ alkynyl, or C₃₋₁₂ cycloalkyl, wherein each C₁₋₁₂ alkyl, C₁₋₁₂ haloalkyl, C₂₋₁₂ alkenyl, C₂₋₁₂ alkynyl, or C₃₋₁₂ cycloalkyl is optionally independently substituted with one or more R¹⁰⁰ as valency permits;
R⁴⁰ is hydrogen, C₁₋₁₂ alkyl, C₁₋₁₂ haloalkyl, C₂₋₁₂ alkenyl, C₂₋₁₂ alkynyl, or C₃₋₁₂ cycloalkyl, wherein each C₁₋₁₂ alkyl, C₁₋₁₂ haloalkyl, C₂₋₁₂ alkenyl, C₂₋₁₂ alkynyl, or C₃₋₁₂ cycloalkyl is optionally independently substituted with one or more R¹⁰⁰ as valency permits;
each of R⁵⁰ and R⁵¹ is independently halo, cyano, nitro, —OR¹⁷⁰, —SR¹⁷⁰, —NR¹⁷⁰R¹⁸⁰, C₁₋₁₂ alkyl, C₁₋₁₂ haloalkyl, C₂₋₁₂ alkenyl, or C₂₋₁₂ alkynyl; wherein each C₁₋₁₂ alkyl, C₁₋₁₂ haloalkyl, C₂₋₁₂ alkenyl, or C₂₋₁₂ alkynyl is independently optionally substituted with one or more halo, hydroxyl or amino as valency permits;
each R¹⁰⁰ is independently oxo, halo, cyano, nitro, —OR¹⁷⁰, —SR¹⁷⁰, —SF₅, —NR¹⁷⁰R¹⁸⁰, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷⁰, —C(═O)OR¹⁷⁰, —OC(═O)OR¹⁷⁰, —OC(═O)R¹⁷⁰, —C(═O)NR¹⁷⁰R¹⁸⁰, —OC(═O)NR¹⁷⁰R¹⁸⁰, —NR¹⁷⁰C(═O)NR¹⁷⁰R¹⁸⁰, —S(═O)₁₋₂R¹⁷⁰, —S(═O)₁₋₂NR¹⁷⁰R¹⁸⁰, —NR¹⁷⁰S(═O)₁₋₂R¹⁸⁰, —NR¹⁷⁰S(═O)₁₋₂NR¹⁷⁰R¹⁸⁰, —NR¹⁷⁰C(═O)R¹⁸⁰, or —NR¹⁷⁰C(═O)OR¹⁸⁰, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each of R¹⁷⁰ and R¹⁸⁰ is independently hydrogen or C₁₋₁₂ alkyl optionally substituted with oxo, halo, hydroxyl, or amino as valency permits,
   or R¹⁷⁰ and R¹⁸⁰ are taken together with the atoms to which they are attached to form heterocyclyl optionally substituted by halo or C₁₋₁₂ alkyl optionally substituted by oxo, halo, hydroxyl, or amino.

In certain embodiments, B¹ is

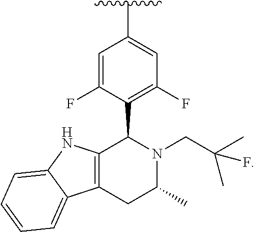 [SMILES: *c1cc(F)c([C@@H]2c3[nH]c4ccccc4c3C[C@@H](C)N2CC(C)(C)F)c(F)c1]

In certain embodiments, B¹ is of Formula IIB′ or IIB″:

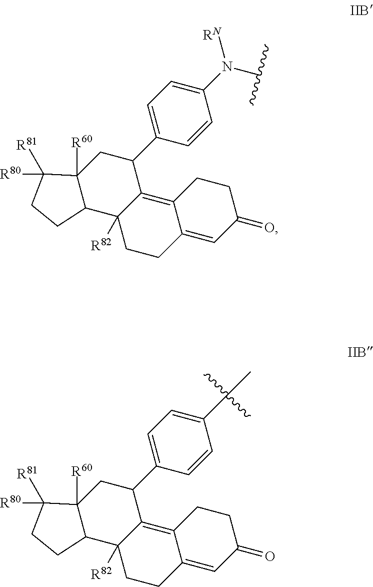

wherein:
the wavy bond represents the point of connection to L¹;
R^{N} is H or C₁₋₁₂ alkyl;
R⁶⁰ is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O)R¹⁰², —NR¹⁰¹C(═O)OR¹⁰², each optionally independently substituted with one or more R¹⁰⁰ as valency permits;
R⁸⁰ is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O)R¹⁰², —NR¹⁰¹C(═O)OR¹⁰², each optionally independently substituted with one or more R¹⁰⁰ as valency permits;
R⁸¹ is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O)R¹⁰², —NR¹⁰¹C(═O)OR¹⁰², each optionally independently substituted with one or more R¹⁰⁰ as valency permits;
or R⁸⁰ and R⁸¹ are taken together with the atom to which they are attached to form heterocyclyl optionally substituted by halo or C₁₋₁₂ alkyl optionally substituted by oxo, halo, hydroxyl or amino;
R⁸² is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O) R¹⁰², —NR¹⁰¹C(═O)OR¹⁰², each optionally independently substituted with one or more R¹⁰⁰ as valency permits;
each of R¹⁰¹ and R¹⁰² is independently hydrogen, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, or 5- to 12-membered heteroaryl, each independently optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
each R¹⁰⁰ is independently oxo, halo, cyano, nitro, —OR¹⁷⁰, —SR¹⁷⁰, —SF₅, —NR¹⁷⁰R¹⁸⁰, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷⁰, —C(═O)OR¹⁷⁰, —OC(═O)OR¹⁷⁰, —OC(═O)R¹⁷⁰, —C(═O)NR¹⁷⁰R¹⁸⁰, —OC(═O)NR¹⁷⁰R¹⁸⁰, —NR¹⁷⁰C(═O)NR¹⁷⁰R¹⁸⁰, —S(═O)₁₋₂R¹⁷⁰, —S(═O)₁₋₂NR¹⁷⁰R¹⁸⁰, —NR¹⁷⁰S(═O)₁₋₂R¹⁸⁰, —NR¹⁷⁰S(═O)₁₋₂NR¹⁷⁰R¹⁸⁰, —NR¹⁷⁰C(═O)R¹⁸⁰, or —NR¹⁷⁰C(═O)OR¹⁸⁰, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each of R¹⁷⁰ and R¹⁸⁰ is independently hydrogen or C₁₋₁₂ alkyl optionally substituted with oxo, halo, hydroxyl or amino as valency permits,
   or R¹⁷⁰ and R¹⁸⁰ are taken together with the atoms to which they are attached to form heterocyclyl optionally substituted by halo or C₁₋₁₂ alkyl optionally substituted by oxo, halo, hydroxyl or amino.

In certain embodiments, B¹ is of Formula IIB′. In certain embodiments, B¹ is of Formula IIB″.

In certain embodiments, R^{N} is methyl.

In certain embodiments, B¹ is of Formula IIB:

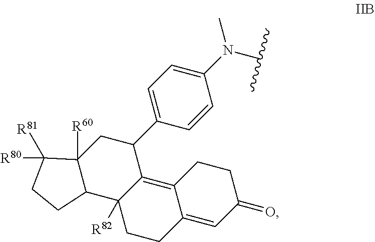

wherein:
the wavy bond represents the point of connection to L¹;
R⁶⁰ is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O)R¹⁰². —NR¹⁰¹C(═O)OR¹⁰², each optionally independently substituted with one or more R¹⁰⁰ as valency permits;
R⁸⁰ is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O)R¹⁰², —NR¹⁰¹C(═O)OR¹⁰², each optionally independently substituted with one or more R¹⁰⁰ as valency permits;
R⁸¹ is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O)R¹⁰², —NR¹⁰¹C(═O)OR¹⁰², each optionally independently substituted with one or more R¹⁰⁰ as valency permits;
or R⁸⁰ and R⁸¹ are taken together with the atom to which they are attached to form heterocyclyl optionally substituted by halo or C₁₋₁₂ alkyl optionally substituted by oxo, halo, hydroxyl or amino;
R⁸² is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O)R¹⁰², —NR¹⁰¹C(═O)OR¹⁰², each optionally independently substituted with one or more R¹⁰⁰ as valency permits;
each of R¹⁰¹ and R¹⁰² is independently hydrogen, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, or 5- to 12-membered heteroaryl, each independently optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
each R¹⁰⁰ is independently oxo, halo, cyano, nitro, —OR¹⁷⁰, —SR¹⁷⁰, —SF₅, —NR¹⁷⁰R¹⁸⁰, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷⁰, —C(═O)OR¹⁷⁰, —OC(═O)OR¹⁷⁰, —OC(═O)R¹⁷⁰, —C(═O)NR¹⁷⁰R¹⁸⁰, —OC(═O)NR¹⁷⁰R¹⁸⁰, —NR¹⁷⁰C(═O)NR¹⁷⁰R¹⁸⁰, —S(═O)₁₋₂R¹⁷⁰, —S(═O)₁₋₂NR¹⁷⁰R¹⁸⁰, —NR¹⁷⁰S(═O)₁₋₂R¹⁸⁰, —NR¹⁷⁰S(═O)₁₋₂NR¹⁷⁰R¹⁸⁰, —NR¹⁷⁰C(═O)R¹⁸⁰, or —NR¹⁷⁰C(═O)OR¹⁸⁰, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each of R¹⁷⁰ and R¹⁸⁰ is independently hydrogen or C₁₋₁₂ alkyl optionally substituted with oxo, halo, hydroxyl or amino as valency permits,
   or R¹⁷⁰ and R¹⁸⁰ are taken together with the atoms to which they are attached to form heterocyclyl optionally substituted by halo or C₁₋₁₂ alkyl optionally substituted by oxo, halo, hydroxyl or amino.

In certain embodiments, R⁸² is hydrogen. In certain embodiments, R⁸² is C₁₋₁₂ alkyl. In certain embodiments, R⁸² is methyl.

In certain embodiments, B¹ is

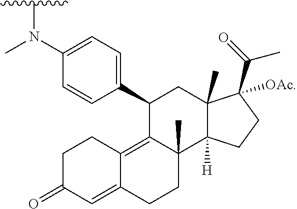

In certain embodiments, B¹ is

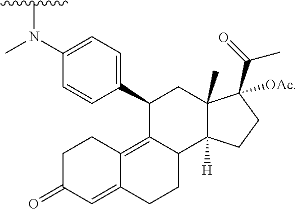 [SMILES: *N(C)c1ccc([C@H]2C[C@@]3(C)[C@@H](CC[C@]3(OC(C)=O)C(C)=O)C3CCC4=CC(=O)CCC4=C32)cc1]

In certain embodiments, B¹ is

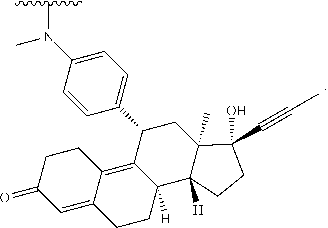 [SMILES: *N(C)c1ccc([C@@H]2C[C@]3(C)[C@H](CC[C@]3(O)C#CC)[C@H]3CCC4=CC(=O)CCC4=C23)cc1]

In certain embodiments, B¹ is

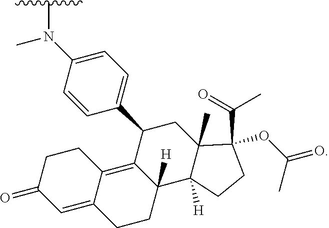

In certain embodiments, B¹ is

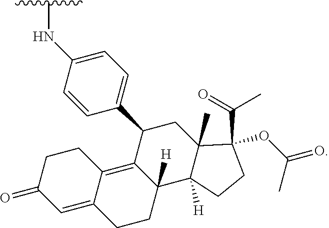

In certain embodiments, B¹ is

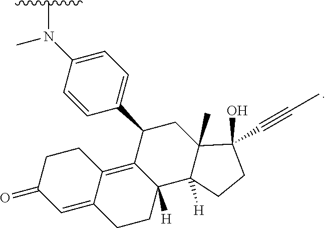

In certain embodiments, B¹ is of Formula IIC′:

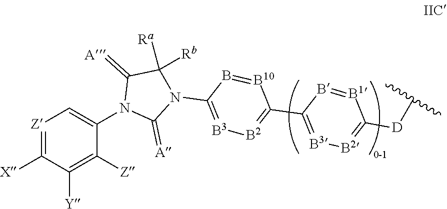

wherein:
the wavy bond represents the point of connection to L¹;
A″ and A′″ are each independently O or S;
R^{a} and R^{b} are each independently CH₃ or CH₂CH₃; or R^{a} and R^{b} together with the atom to which they are attached form a C₃₋₆ cycloalkyl, oxirane, oxetane or tetrahydrofuran;
B, B¹⁰, B², B³, B′, B^{1′}, B^{2′} and B^{3′} are each independently CR^{c} or N;
each R^{c} is independently hydrogen, fluoro, CN, or methyl;
D is absent, NH, O, S, CH₂, —NH(C═O)—, —(C═O)NH—, or C═O;
X″ is CN, halo, or NO₂;
Y″ is CH₃, CH₂R^{d}, CHF₂, or CF₃;
R^{d} is halo;
Z″ is H, C₁₋₂ alkyl, C₂ alkenyl or NO₂; or
X″ and Y″ together form a

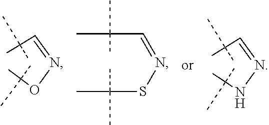

wherein the broken lines indicate bonds to the ring;
or Y″ and Z″ together form a

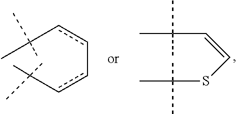

wherein each [symbol] is a single or double bond, and wherein the broken lines indicate bonds to the ring; and
Z′ is CH or N.

In certain embodiments, D is NH, O, S, CH₂, —NH(C═O)—, —(C═O)NH—, or C═O.

In certain embodiments, B¹ is of Formula IIC:

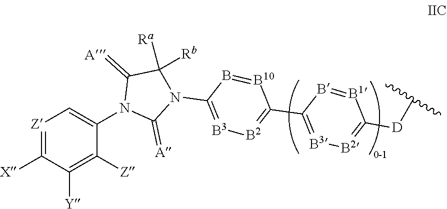

wherein:
the wavy bond represents the point of connection to L¹;
A″ and A′″ are each independently O or S;
R^{a} and R^{b} are each independently CH₃ or CH₂CH₃; or R^{a} and R^{b} together with the atom to which they are attached form a C₃₋₆ cycloalkyl, oxirane, oxetane or tetrahydrofuran;
B, B¹⁰, B², B³, B′, B^{1′}, B^{2′}, and B^{3′} are each independently CR^{c} or N;
each R^{c} is independently hydrogen, fluoro, CN, or methyl;
D is NH, O, S, CH₂, —NH(C═O)—, —(C═O)NH—, or C═O;
X″ is CN, halo, or NO₂;
Y″ is CH₃, CH₂R^{d}, CHF₂, or CF₃;
R^{d} is halo;
Z″ is H, C₁₋₂ alkyl, C₂ alkenyl, or NO₂; or
X″ and Y″ together form a

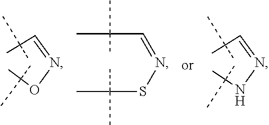

wherein the broken lines indicate bonds to the ring;
or Y″ and Z″ together form a

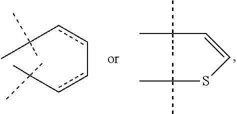

wherein each [symbol] is a single or double bond, and wherein the broken lines indicate bonds to the ring; and
Z′ is CH or N.

In certain embodiments, B¹ is

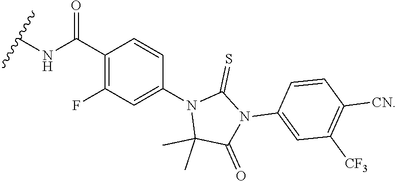

In certain embodiments, B¹ is

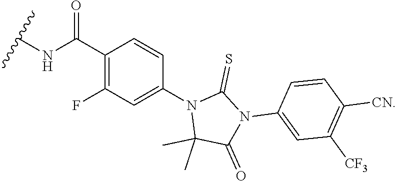

In certain embodiments, B¹ is

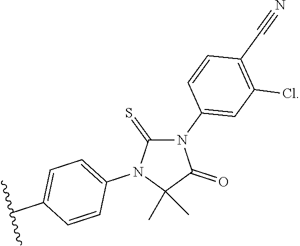 [SMILES: *c1ccc(N2C(=S)N(c3ccc(C#N)c(Cl)c3)C(=O)C2(C)C)cc1]

In certain embodiments, B¹ is of Formula IID′:

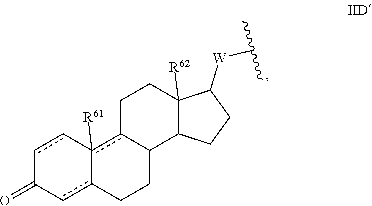

wherein:
W is O, S, or NH;
each [symbol] is independently a double bond or a single bond;
each of R⁶¹ and R⁶² is independently hydrogen, C₁₋₁₂ alkyl, C₂₋₁₂ alkenyl, C₂₋₁₂ alkynyl, or C₃₋₁₂ cycloalkyl, wherein each C₁₋₁₂ alkyl, C₂₋₁₂ alkenyl, C₂₋₁₂ alkynyl, or C₃₋₁₂ cycloalkyl is optionally independently substituted with one or more R¹⁰⁰ as valency permits;
each R¹⁰⁰ is independently oxo, halo, cyano, nitro, —OR¹⁷⁰, —SR¹⁷⁰, —SF₅, —NR¹⁷⁰R¹⁸⁰, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷⁰, —C(═O)OR¹⁷⁰, —OC(═O)OR¹⁷⁰, —OC(═O)R¹⁷⁰, —C(═O)NR¹⁷⁰R¹⁸⁰, —OC(═O)NR¹⁷⁰R¹⁸⁰, —NR¹⁷⁰C(═O)NR¹⁷⁰R¹⁸⁰, —S(═O)₁₋₂R¹⁷⁰, —S(═O)₁₋₂NR¹⁷⁰R¹⁸⁰, —NR¹⁷⁰S(═O)₁₋₂R¹⁸⁰, —NR¹⁷⁰S(═O)₁₋₂NR¹⁷⁰R¹⁸⁰, —NR¹⁷⁰C(═O)R¹⁸⁰, or —NR¹⁷⁰C(═O)OR¹⁸⁰, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each of R¹⁷⁰ and R¹⁸⁰ is independently hydrogen or C₁₋₁₂ alkyl optionally substituted with oxo, halo, hydroxyl or amino as valency permits,
   or R¹⁷⁰ and R¹⁸⁰ are taken together with the atoms to which they are attached to form heterocyclyl optionally substituted by halo or C₁₋₁₂ alkyl optionally substituted by oxo, halo, hydroxyl or amino.

In certain embodiments, B¹ is of Formula IID:

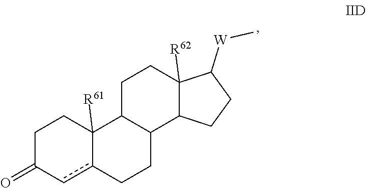

wherein:
W is O, S, or NH;
[symbol] is a double bond or a single bond;
each of R⁶¹ and R⁶² is independently hydrogen, C₁₋₁₂ alkyl, C₂₋₁₂ alkenyl, C₂₋₁₂ alkynyl, or C₃₋₁₂ cycloalkyl, wherein each C₁₋₁₂ alkyl, C₂₋₁₂ alkenyl, C₂₋₁₂ alkynyl, or C₃₋₁₂ cycloalkyl is optionally independently substituted with one or more R¹⁰⁰ as valency permits;
each R¹⁰⁰ is independently oxo, halo, cyano, nitro, —OR¹⁷⁰, —SR¹⁷⁰, —SF₅, —NR¹⁷⁰R¹⁸⁰, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷⁰, —C(═O)OR¹⁷⁰, —OC(═O)OR¹⁷⁰, —OC(═O)R¹⁷⁰, —C(═O)NR¹⁷⁰R¹⁸⁰, —OC(═O)NR¹⁷⁰R¹⁸⁰, —NR¹⁷⁰C(═O)NR¹⁷⁰R¹⁸⁰, —S(═O)₁₋₂R¹⁷⁰, —S(═O)₁₋₂NR¹⁷⁰R¹⁸⁰, —NR¹⁷⁰S(═O)₁₋₂R¹⁸⁰, —NR¹⁷⁰S(═O)₁₋₂NR¹⁷⁰R¹⁸⁰, —NR¹⁷⁰C(═O)R¹⁸⁰, or —NR¹⁷⁰C(═O)OR¹⁸⁰, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each of R¹⁷⁰ and R¹⁸⁰ is independently hydrogen or C₁₋₁₂ alkyl optionally substituted with oxo, halo, hydroxyl, or amino as valency permits,
   or R¹⁷⁰ and R¹⁸⁰ are taken together with the atoms to which they are attached to form heterocyclyl optionally substituted by halo or C₁₋₁₂ alkyl optionally substituted by oxo, halo, hydroxyl, or amino.

In certain embodiments, B¹ is

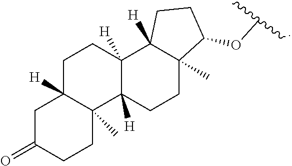 [SMILES: *O[C@H]1CC[C@H]2[C@@H]3CC[C@H]4CC(=O)CC[C@]4(C)[C@H]3CC[C@]12C]

In certain embodiments, B¹ is

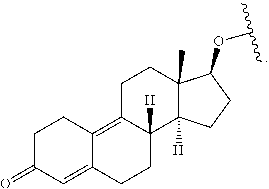

In certain embodiments, B¹ is of Formula IIE:

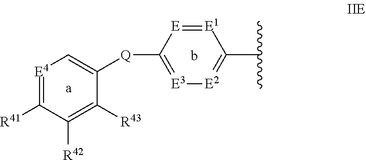

wherein:
the wavy bond refers to the point of connection to L;
Q is

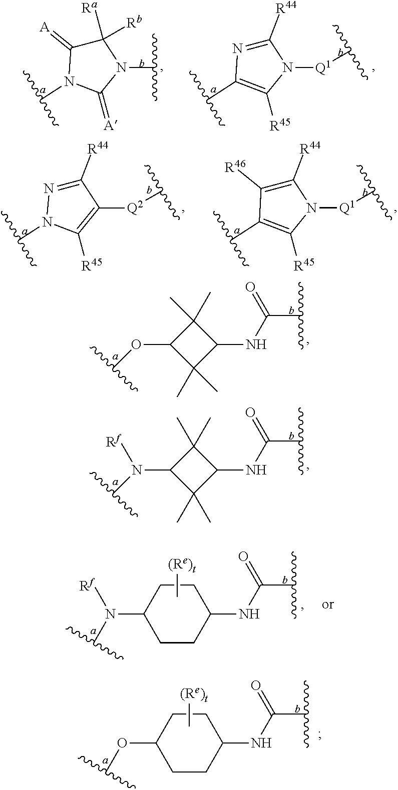

wherein bond a is attached to ring a and bond b is attached to ring b;
R^{a} and R^{b} are each independently —CH₃ or —CH₂CH₃; or R^{a} and R^{b} together with the atom to which they are attached form a C₃₋₅ cycloalkyl, oxiranyl, oxetanyl, or tetrahydrofuranyl;
A and A′ are each independently O or S;
E, E¹, E², and E³ are each independently CR^{c} or N, and each R^{c} is independently hydrogen, halo, CN, or methyl;
E⁴ is CF, CH or N;
Q¹ is a bond, CH₂, C═O, or (C═O)NH;
Q² is NH, O, S, CH₂, NH(C═O), C(═O)NH, or C═O;
R⁴⁴, R⁴⁵ and R⁴⁶ are each independently hydrogen, CN, or C₁₋₂ alkyl;
t is 0, 1, 2, 3, or 4;
each of R^{e} and R^{f} is independently halo, cyano, C₁₋₄ alkyl, or C₁₋₄ haloalkyl;
R⁴¹ is halo, CN, or NO₂;
R⁴² is halo, CH₃, CH₂F, CHF₂, or CF₃; or

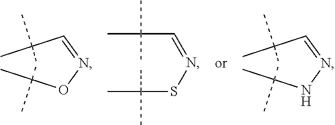

R⁴¹ and R⁴² together form a wherein the broken lines indicate bonds to ring a;
R⁴³ is hydrogen, halo, C₁₋₂ alkyl, C₂ alkenyl, NO₂, CF₃; or
R⁴² and R⁴³ together form a

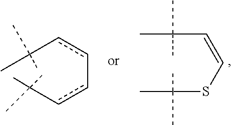

wherein each [symbol] is a single or double bond, and wherein the broken lines indicate bonds to ring a.

In certain embodiments, B¹ is

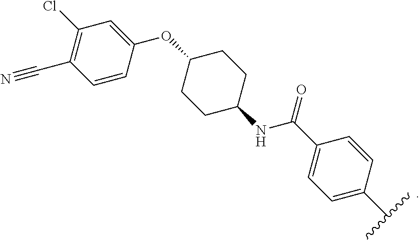 [SMILES: *c1ccc(C(=O)N[C@H]2CC[C@H](Oc3ccc(C#N)c(Cl)c3)CC2)cc1]

In certain embodiments, B¹ is

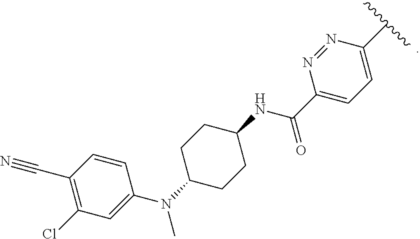 [SMILES: *c1ccc(C(=O)N[C@H]2CC[C@H](N(C)c3ccc(C#N)c(Cl)c3)CC2)nn1]

In certain embodiments, B¹ is

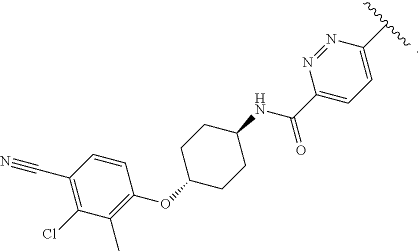 [SMILES: *c1ccc(C(=O)N[C@H]2CC[C@H](Oc3ccc(C#N)c(Cl)c3C)CC2)nn1]

In certain embodiments, B¹ is

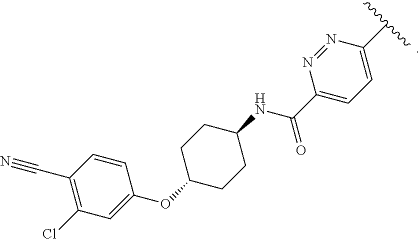 [SMILES: *c1ccc(C(=O)N[C@H]2CC[C@H](Oc3ccc(C#N)c(Cl)c3)CC2)nn1]

In certain embodiments, B¹ is derived from progesterone, enobosarm, bicalutamide, apalutamide, testosterone, dihydrotestosterone, testosterone, 19-nortestosterone, progesterone, andarine, cortisol, prednisone, flutamide, nilutamide, enzalutamide, tamoxifen, toremifene, raloxifene, bazedoxifene, ospemifene, megestrol acetate, estramustine, abiraterone, LGD-2941, BMS-564929, ostarine, ulipristal acetate, asoprisnil (J867), mifepristone, telapristone (CDB-4124, Proellex, Progenta), or an analog thereof.

In certain embodiments, B¹ comprises a nuclear receptor-targeting epitope derived from:

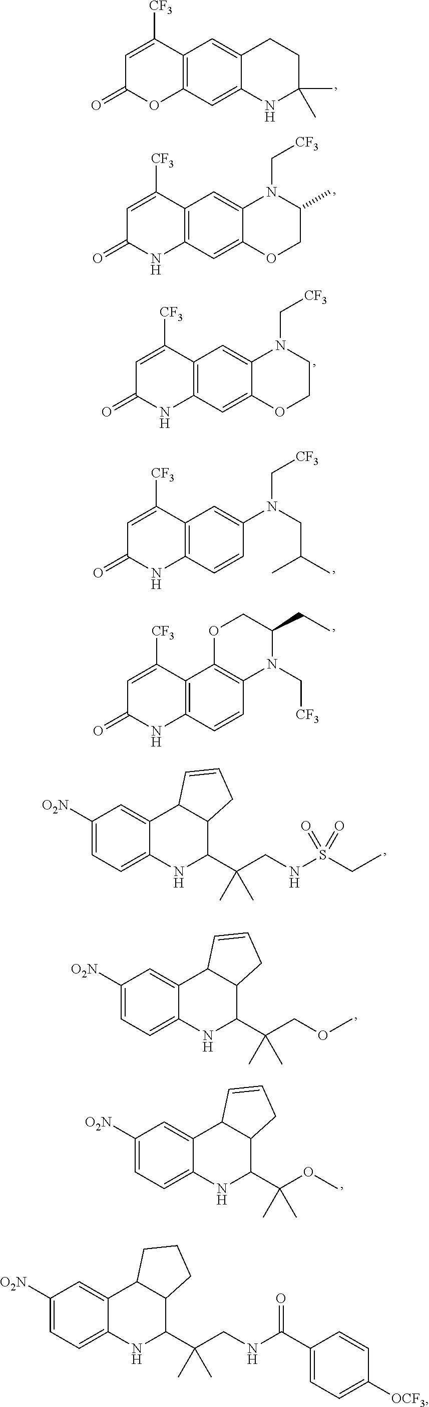

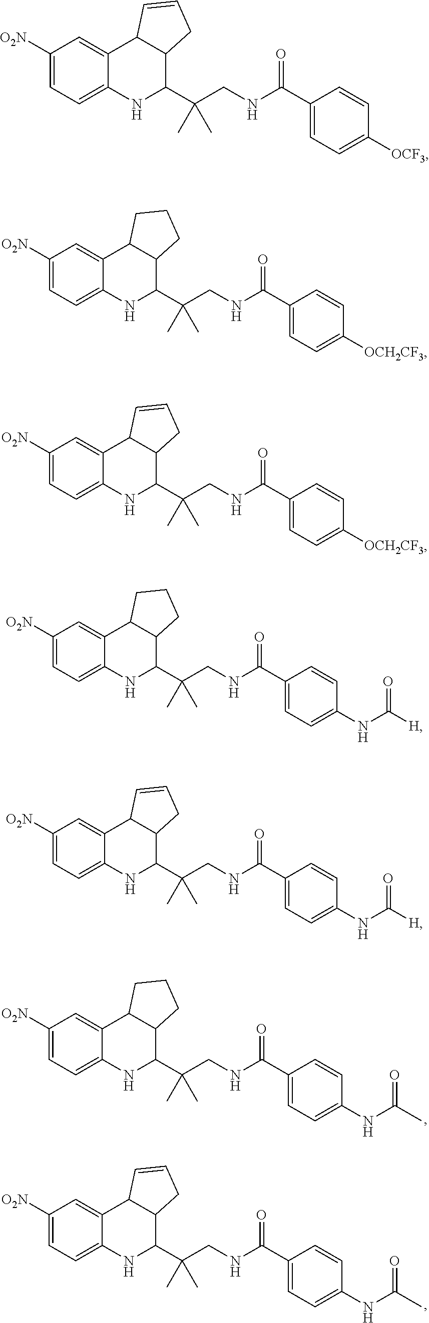

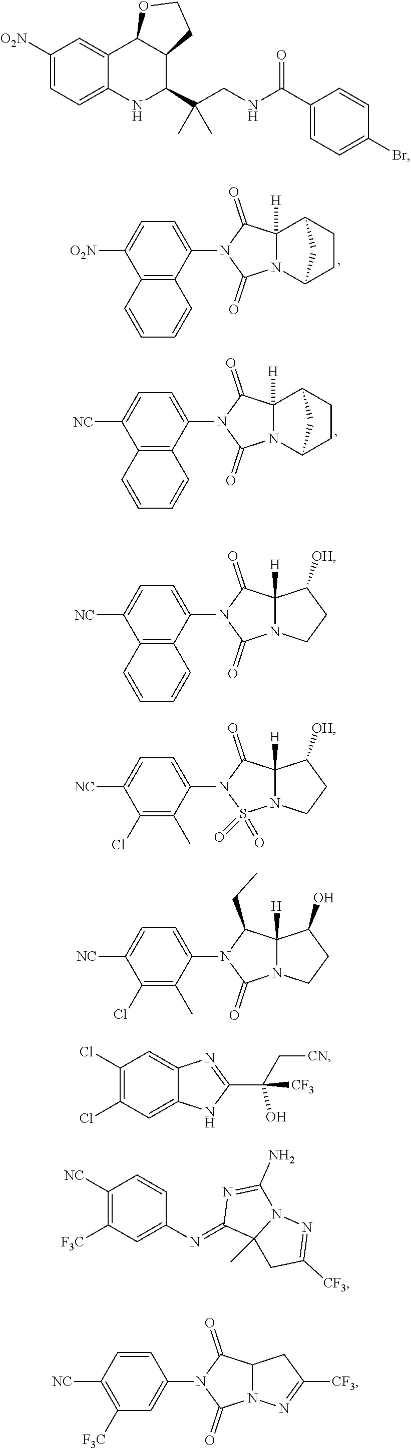

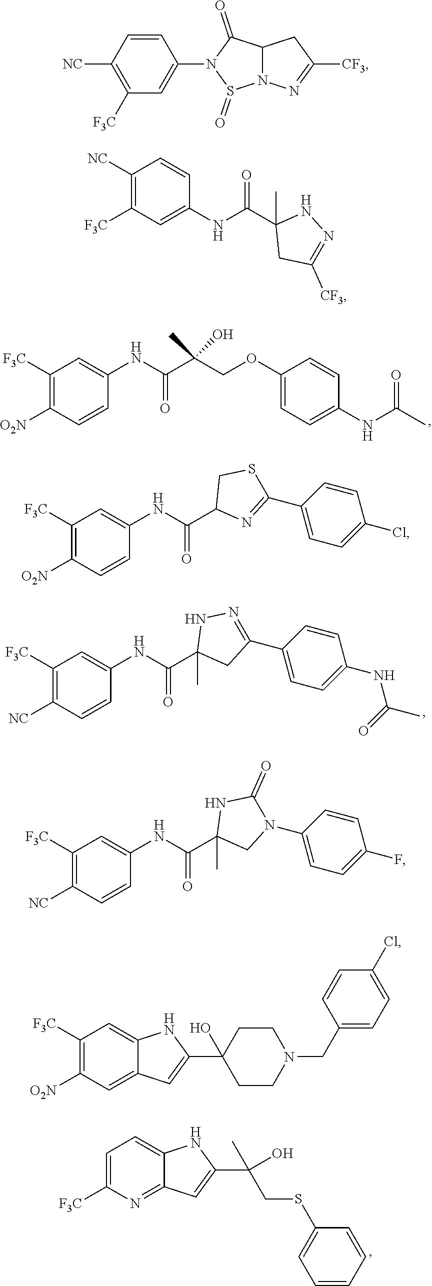

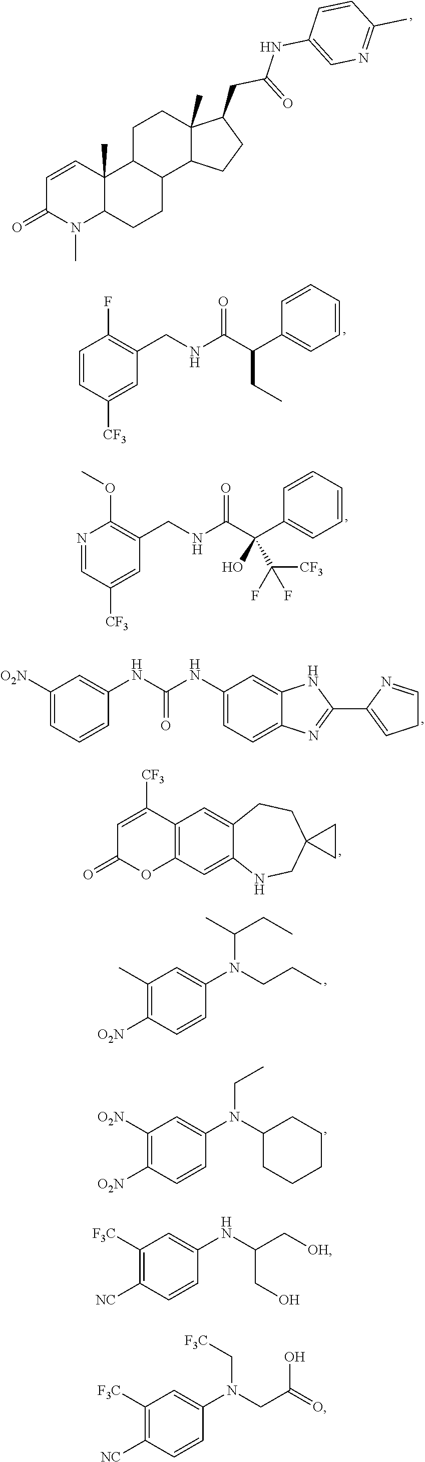

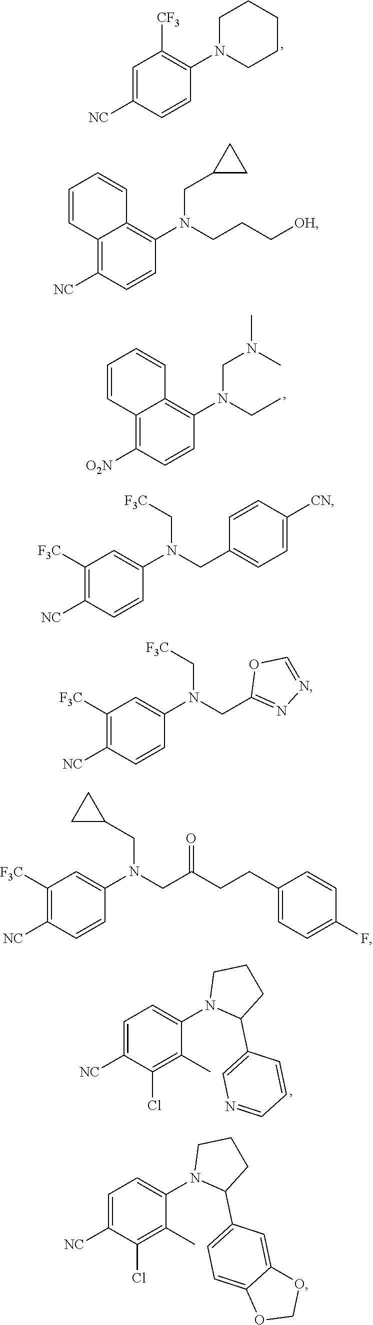

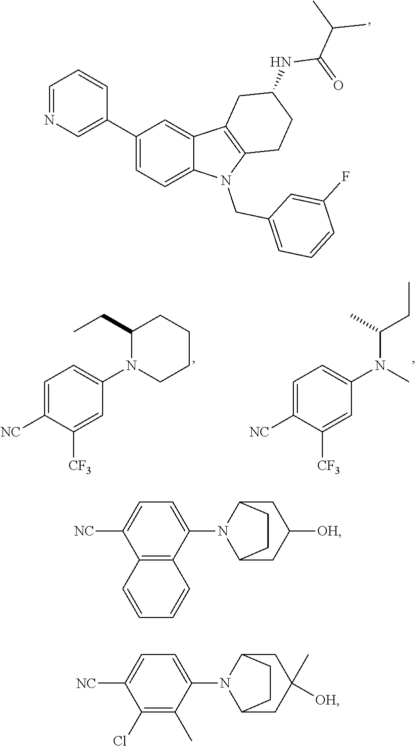

or a stereoisomer or a mixture of stereoisomers thereof or an analog thereof, wherein at least one hydrogen atom is replaced by a direct covalent bond to A¹, optionally via a linking moiety.

These and other selective androgen receptor modulator (SARMs) which can be used as a nuclear steroid receptor-targeting epitope in B¹ described herein can be found in U.S. Pat. Nos. 6,462,038, 6,777,427, WO2001/027086, WO2004/013104, WO2004/000816, WO2004/0113309, US2006/0211756, US2006/0063819, US2005/245485, US2005/250741, US2005/277681, WO2006/060108, WO2004/041277, WO2003/034987, US2006/0148893, US2006/0142387, WO2005/000795, WO2005/085185, WO2006/133216, WO2006/044707, WO2006/124447, WO2007/002181, WO2005/108351, WO2005/115361, and US2006/0160845.

In certain embodiments, B¹ is a selective estrogen receptor modulator (SERM). In certain embodiments, B¹ comprises an epitope derived from anordrin, bazedoxifene, broparestrol (Acnestrol), clomifene (Clomid), cyclofenil (Sexovid), lasofoxifene (Fablyn), ormeloxifene (Centron, Novex, Novex-DS, Sevista), ospemifene (Osphena, deaminohydroxytoremifene), raloxifene (Evista), tamoxifen (Nolvadex), toremifene (Fareston; 4-chlorotamoxifen), acolbifene, afimoxifene (4-hydroxytamoxifen; metabolite of tamoxifen), elacestrant, enclomifene ((E)-clomifene), endoxifen (4-hydroxy-N-desmethyltamoxifen; metabolite of tamoxifen), zuclomifene ((Z)-clomifene), bazedoxifene, arzoxifene, brilanestrant, clomifenoxide (clomiphene N-oxide; metabolite of clomifene), droloxifene (3-hydroxytamoxifen), etacstil, fispemifene, GW-7604 (4-hydroxyetacstil), idoxifene (pyrrolidino-4-iodotamoxifen), levormeloxifene ((L)-ormeloxifene), miproxifene, nafoxidine, nitromifene (CI-628), panomifene, pipendoxifene (ERA-923), trioxifene, keoxifene, LY117018, onapristone, fareston (toremifine citrate) or zindoxifene (D-16726), or an analog thereof.

In certain embodiments, the SERM is classified structurally as a triphenylethylene (tamoxifen, clomifene, toremifene, droloxifene, idoxifene, ospemifene, fispemifene, afimoxifene, etc., or an analog thereof), a benzothiophene (raloxifene, arzoxifene, etc., or an analog thereof), an indole (bazedoxifene, zindoxifene, pipendoxifene, etc., or an analog thereof), a tetrahydronaphthalene (lasofoxifene, nafoxidine, etc., or an analog thereof), or a benzopyran (acolbifene, ormeloxifene, levormeloxifene, etc., or an analog thereof).

In certain embodiments, B¹ is a selective estrogen receptor downregulator (SERD). In certain embodiments, the compound comprises at least one nuclear steroid receptor-targeting epitope independently comprises an epitope derived from fulvestrant, brilanestrant (ARN-810), etacstil (GW5638), AZD9496, giredestrant (GDC-9545) or GW7604.

In certain embodiments, B¹ is a selective progesterone receptor modulator (SPRM). In certain embodiments, B comprises an epitope derived from ulipristal acetate, asoprisnil (J867), mifepristone, telapristone (CDB-4124, Proellex, Progenta), or an analog thereof.

In certain embodiments, B¹ comprises an epitope derived from, estrogen, estetrol, estriol, estrone, progesterone, enobosarm, bicalutamide, apalutamide, testosterone, dihydrotestosterone, estradiol, flutamide, nilutamide, enzalutamide, tamoxifen, toremifene, raloxifene, bazedoxifene, ospemifene, megestrol acetate, estramustine, abiraterone, LGD-2941, BMS-564929, ostarine, or an analog thereof.

In certain embodiments, at least one nuclear steroid receptor-targeting epitope is an androgen receptor-targeting epitope, and comprises:

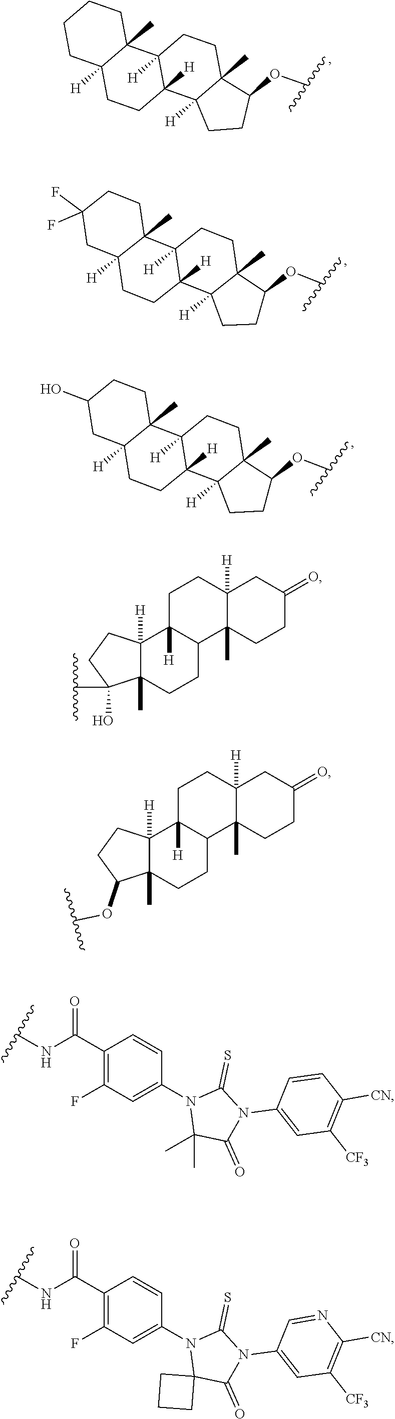

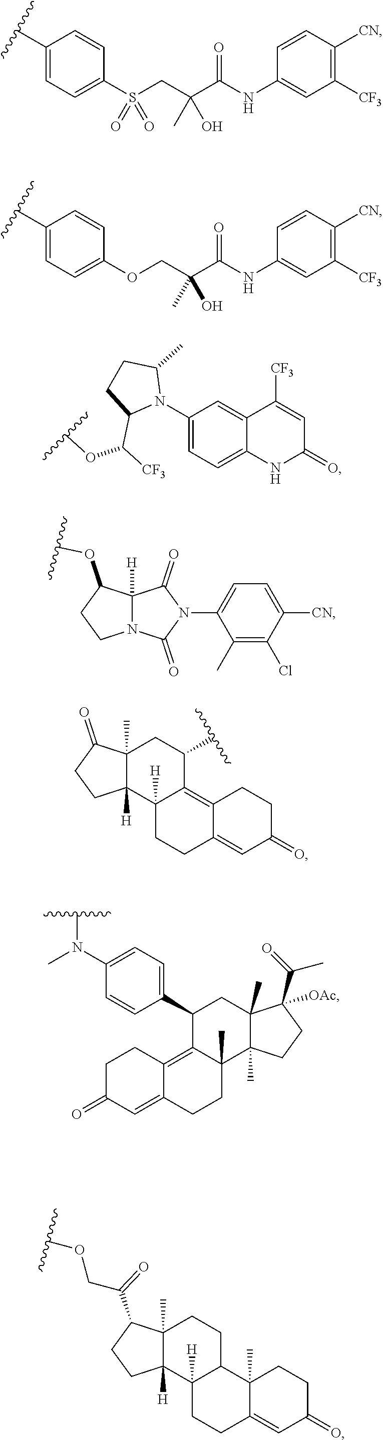

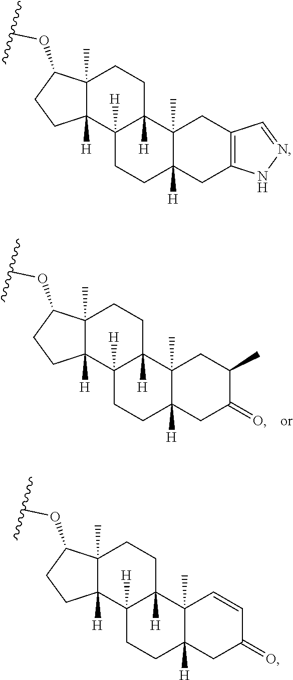

or a stereoisomer or a mixture of stereoisomers thereof or an analog thereof, where the wavy line indicates the point of attachment to the nuclear payload, optionally via a linking moiety.

In certain embodiments, at least one nuclear steroid receptor-targeting epitope is an estrogen receptor-targeting epitope, and comprises:

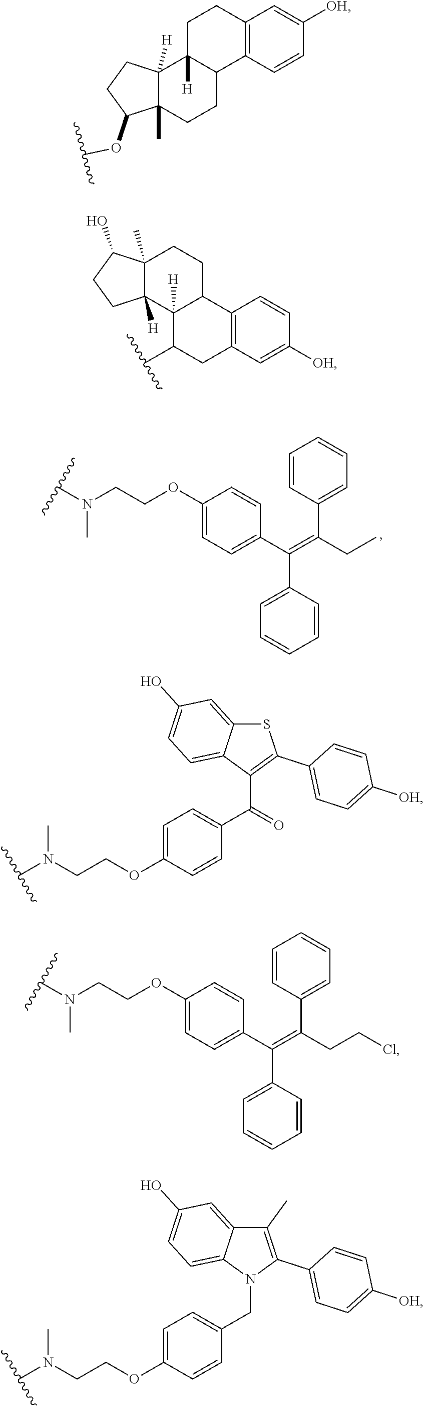

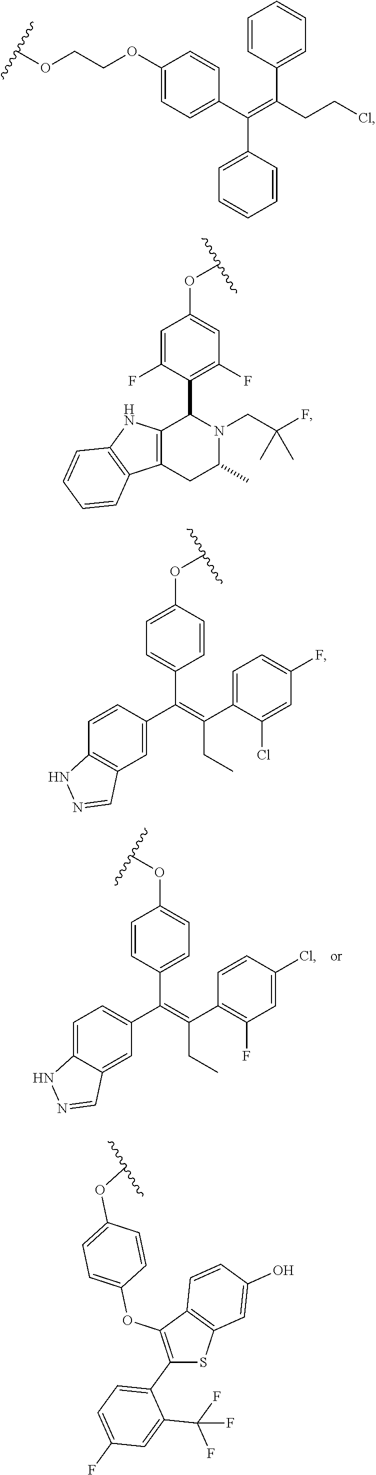 [SMILES: *OCCOc1ccc(/C(=C(/CCCl)c2ccccc2)c2ccccc2)cc1 ; *Oc1cc(F)c([C@@H]2c3[nH]c4ccccc4c3C[C@@H](C)N2CC(C)(C)F)c(F)c1 ; *Oc1ccc(/C(=C(/CC)c2ccc(F)cc2Cl)c2ccc3[nH]ncc3c2)cc1 ; *Oc1ccc(/C(=C(/CC)c2ccc(Cl)cc2F)c2ccc3[nH]ncc3c2)cc1 ; *Oc1ccc(Oc2c(-c3ccc(F)cc3C(F)(F)F)sc3cc(O)ccc23)cc1]

or a stereoisomer or a mixture of stereoisomers thereof or an analog thereof, where the wavy line indicates the point of attachment to the nuclear payload, optionally via a linking moiety.

In certain embodiments, at least one nuclear steroid receptor-targeting epitope is an estrogen receptor-targeting epitope, and comprises:

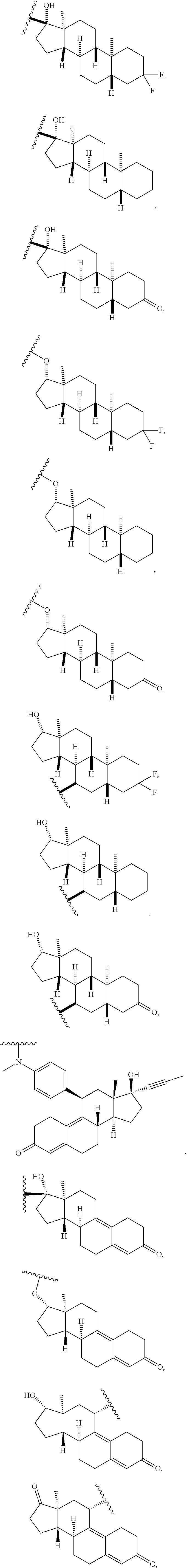

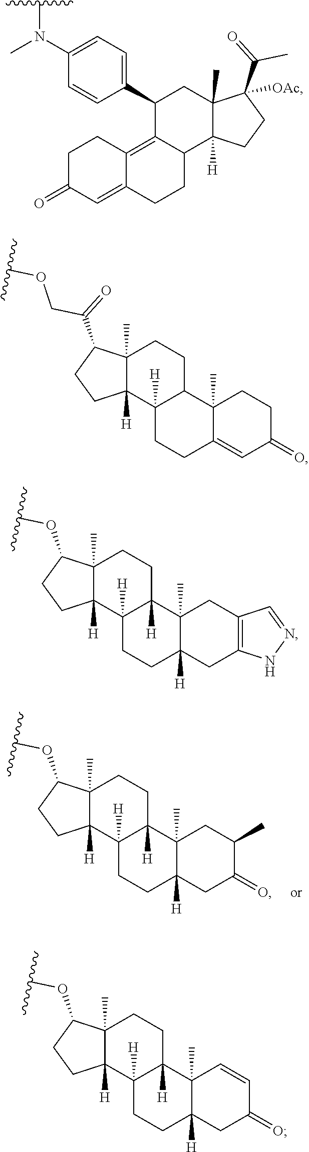

or a stereoisomer or a mixture of stereoisomers thereof or an analog thereof, where the wavy line indicates the point of attachment to the nuclear payload, optionally via a linking moiety.

In certain embodiments, at least one nuclear steroid receptor-targeting epitope comprises:

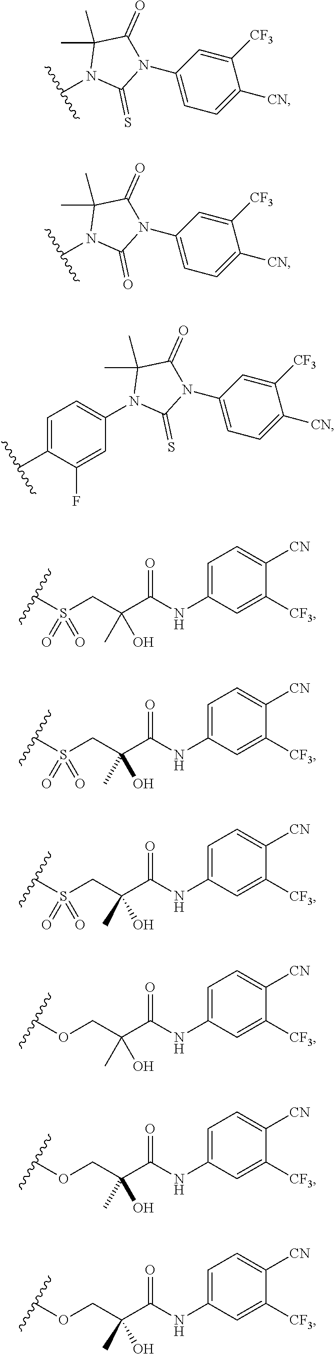

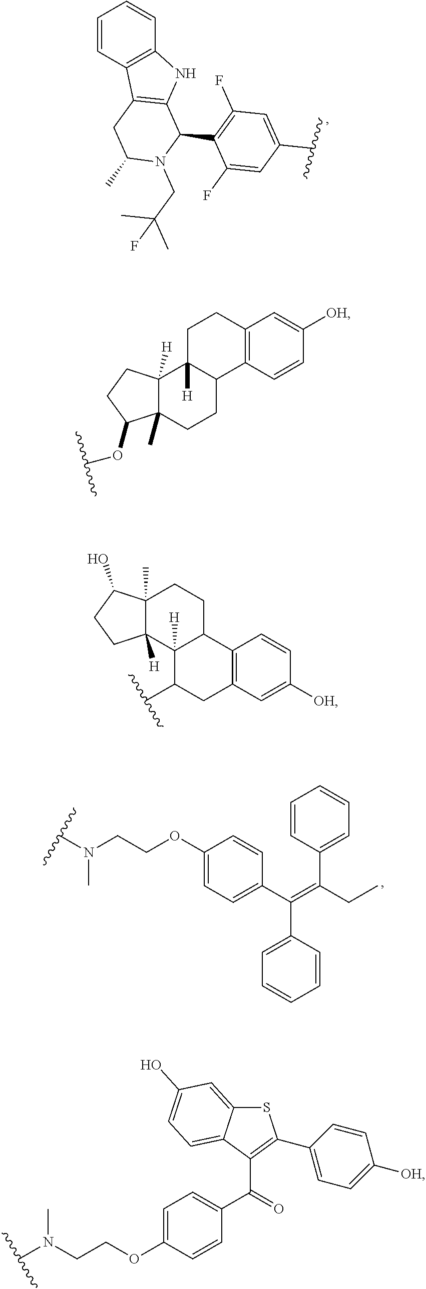

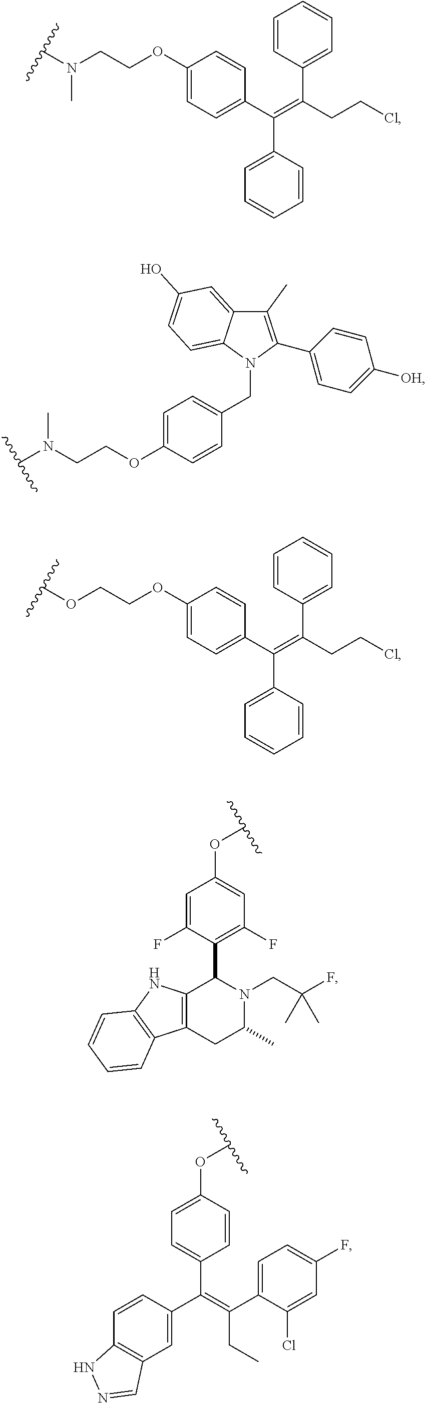

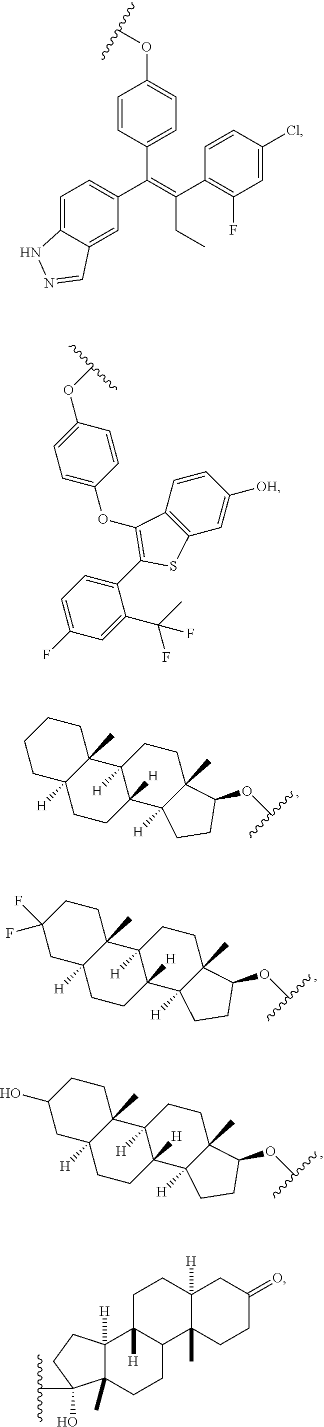

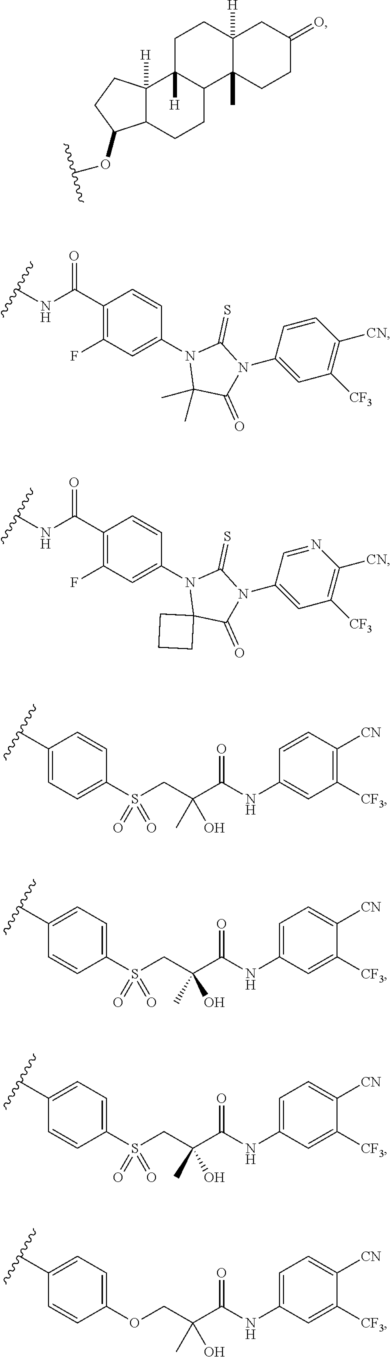

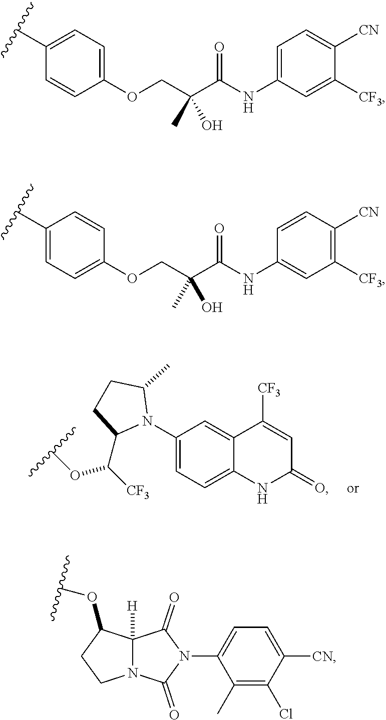

or a stereoisomer or a mixture of stereoisomers thereof or an analog thereof, where the wavy line indicates the point of attachment to the nuclear payload, optionally via a linking moiety.

In certain embodiments, at least one nuclear steroid receptor-targeting epitope comprises:

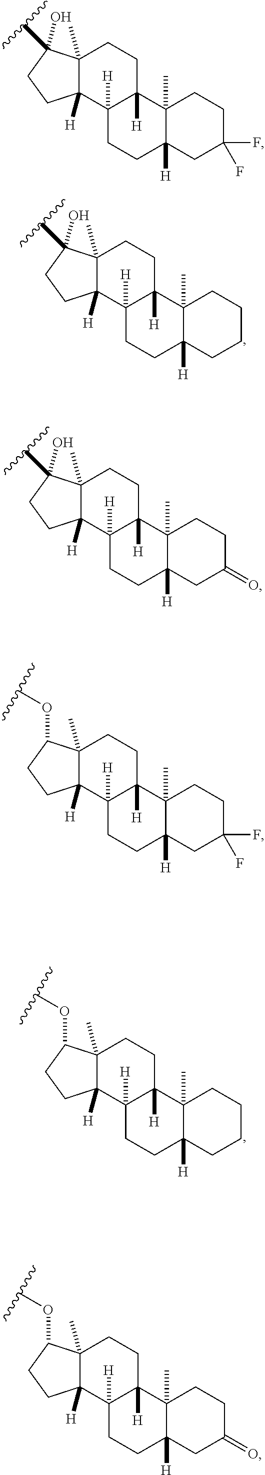

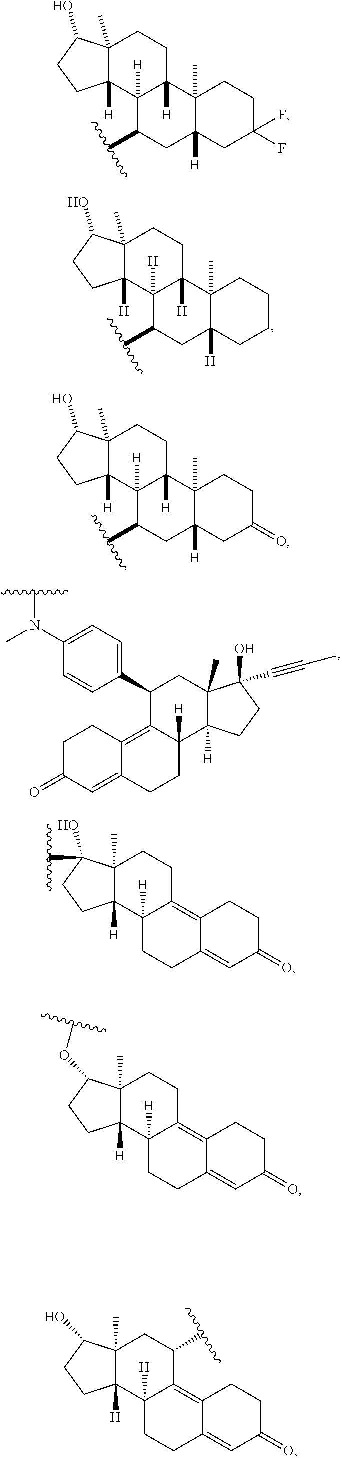

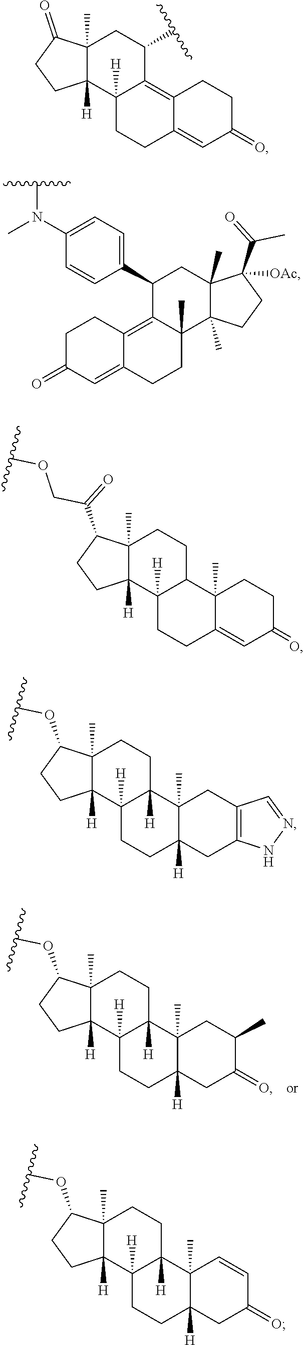

or a stereoisomer or a mixture of stereoisomers thereof or an analog thereof, where the wavy line indicates the point of attachment to the nuclear payload, optionally via a linking moiety.

In certain embodiments, the nuclear steroid receptor-targeting epitope is not, or does not contain, a peptide, protein, nanoparticle or antibody.

### Linking Moiety

The “linking moiety” of any compounds described herein can be biocleavable (e.g., acid labile) or non-biocleavable. Linking moieties can be linear, branched, saturated, unsaturated, all-carbon or heteroatomic. Linking moieties can also contain one or more rings that are fused, saturated, unsaturated, as well as be all-carbon or heteroatomic. In certain embodiments, the linking moiety is a non-biocleavable linking moiety. In certain embodiments, the linking moiety is a biocleavable linking moiety. In certain embodiments, a nuclear payload is bonded to one nuclear steroid receptor-targeting epitope via a non-biocleavable linking moiety and one or more nuclear steroid receptor-targeting epitope(s) via a biocleavable linking moiety. In certain embodiments, the biocleavable linking moiety is an acid-labile linking moiety. In some embodiments, the linking moiety comprises a hydrazone linkage.

It is contemplated that any linking moiety can be used in the compounds described herein, provided that it does not significantly interfere with or disrupt the desired binding of the nuclear payload or the nuclear receptor-targeting epitope.

In certain embodiments, L¹ is of formula:

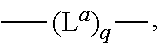

wherein:
each L^{a} is independently W, —NR¹¹⁰—, —O—, —S(O)₀₋₂—, —NR¹¹⁰C(O)—, —C(O)NR¹¹⁰, —NR¹¹⁰C(O)NR¹¹⁰—, —NR¹¹⁰S(O)₂—, —S(O)₂NR¹¹⁰—, —NR¹¹⁰S(O)₂NR¹¹⁰—, —CR¹²⁰═N—NR¹¹⁰—, —NR¹¹⁰—N═CR¹²⁰—, —C(O)—, —OC(O)—, —OC(O)O—, —C(O)O—, C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene, each independently optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl,
   wherein each W is independently

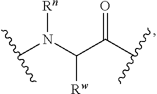

wherein Rⁿ, at each occurrence, is independently H, C₁₋₄ alkyl, or C₁₋₄ haloalkyl; and wherein R^{w}, at each occurrence, is independently H, C₃₋₁₂ cycloalkyl, C₆₋₁₂ aryl optionally substituted with one or more halo or OH, or C₁₋₄ alkyl optionally substituted with one or more independently selected halo, OH, —SH, —S(C₁₋₄ alkyl), —CONH₂, —COOH, —NHC(═NH)NH₂, —NH₂, —NHCOCH₃, —NHCHO, —NHCONH₂, C₆₋₁₂ aryl, 5- to 12-membered heterocycle, or 5- to 12-membered heteroaryl;
each R¹¹⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹²⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl; and
q is an integer from 0 to 40. It is understood that either end of L¹ can be connected to A¹.

In certain embodiments, L¹ is of formula:

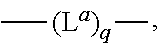

wherein:
each L^{a} is independently —NR¹¹⁰—, —O—, —S(O)₀₋₂—, —NR¹¹⁰C(O)—, —C(O)NR¹¹⁰—, —NR¹¹⁰C(O)NR¹¹⁰—, —NR¹¹⁰S(O)₂—, —S(O)₂NR¹¹⁰—, —NR¹¹⁰S(O)₂NR¹¹⁰—, —CR¹²⁰═N—NR¹¹⁰—, —NR¹¹⁰—N═CR¹²⁰—, —C(O)—, —OC(O)—, —OC(O)O—, —C(O)O—, C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene, each independently optionally substituted with one or more substituents independently selected from oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl;
each R¹¹⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹²⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl; and
q is an integer from 0 to 20.

In certain embodiments, L¹ is of the formula:

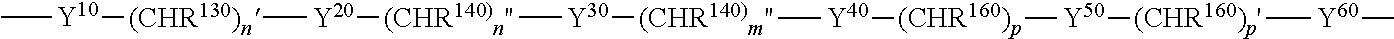

wherein:
each of Y¹⁰, Y²⁰, Y³⁰, Y⁴⁰, Y⁵⁰, and Y⁶⁰ are independently —(W)ₛ—, a bond, —NR¹¹⁰—, —O—, —S(O)₀₋₂—, —NR¹¹⁰C(O)—, —C(O)NR¹¹⁰, —NR¹¹⁰C(O)NR¹¹⁰—, —NR¹¹⁰S(O)₂—, —S(O)₂NR¹¹⁰—, —NR¹¹⁰S(O)₂NR¹¹⁰—, —CR¹²⁰═N—NR¹¹⁰—, —NR¹¹⁰—N═CR¹²⁰, —C(O)—, —OC(O)—, —OC(O)O—, —(CH₂CH₂O)₁₋₅—, —C(O)O—, C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene, each independently optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl;
each W is independently

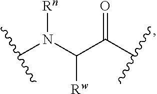

wherein Rⁿ, at each occurrence, is independently H, C₁₋₄ alkyl, or C₁₋₄ haloalkyl; and wherein R^{w}, at each occurrence, is independently H, C₃₋₁₂ cycloalkyl, C₆₋₁₂ aryl optionally substituted with one or more halo or OH, or C₁₋₄ alkyl optionally substituted with one or more independently selected halo, OH, —SH, —S(C₁₋₄ alkyl), —CONH₂, —COOH, —NHC(═NH)NH₂, —NH₂, —NHCOCH₃, —NHCHO, —NHCONH₂, C₆₋₁₂ aryl, 5- to 12-membered heterocycle, or 5- to 12-membered heteroaryl;
each of R¹¹⁰, R¹²⁰, R¹³⁰, R¹⁴⁰, R¹⁵⁰, R¹⁶⁰, and R¹⁷⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl, each independently optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl; and
n′, n″, m″, s, p, and p′ are each independently 0, 1, 2, 3, 4, 5, 6, 7, or 8.

In certain embodiments, L¹ is of the formula:

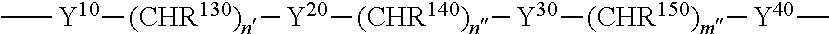

wherein:
each of Y¹⁰, Y²⁰, Y³⁰, and Y⁴⁰ are independently a bond, —NR¹¹⁰—, —O—, —S(O)₀₋₂—, —NR¹¹⁰C(O)—, —C(O)NR¹¹⁰—, —NR¹¹⁰C(O)NR¹¹⁰—, —NR¹¹⁰S(O)₂—, —S(O)₂NR¹¹⁰—, —NR¹¹⁰S(O)₂NR¹¹⁰—, —CR¹²⁰═N—NR¹¹⁰—, —NR¹¹⁰—N═CR¹²⁰, —C(O)—, —OC(O)—, —OC(O)O—, —(CH₂CH₂O)₁₋₅—, —C(O)O—, C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene, each independently optionally substituted with one or more substituents independently selected from oxo, halo, C₁₋₄ alkyl, C₁₋₄ haloalkyl, C₁₋₄ alkoxy, or C₁₋₄ haloalkoxy;
each R¹¹⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹²⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹³⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹⁴⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹⁵⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl; and
n′, n″, and m″ are each independently 0, 1, 2, 3, 4, 5, 6, 7, or 8.

In certain embodiments, at least one W is Val. In certain embodiments, at least one W is Cit. In certain embodiments, s is 2. In certain embodiments, —(W)ₛ— is -Val-Cit-.

In certain embodiments, L¹ is of the formula:

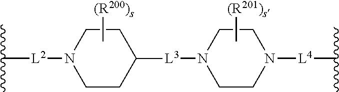

wherein:
each of L², L³, and L⁴ is independently a bond, C₁₋₁₂ alkylene, —NHC(═O)—, —C(═O)NH—, —C(═O)—O—, —O—C(═O)—, or C═O;
each of R²⁰⁰ and R²⁰¹ is independently halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl; and
each of s and s′ is independently 0, 1, 2, 3, or 4.

In certain embodiments, L¹ is of the formula:

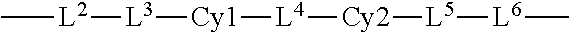

wherein:
each of L², L³, L⁴ L⁵, and L⁶ is independently a bond, C₁₋₁₂ alkylene, —O—, —NHC(═O)—, —C(═O)NH—, —C(═O)—O—, —O—C(═O)—, or C═O, wherein one or more carbon atoms in the C₁₋₁₂ alkylene are optionally replaced with oxygen;
Cy1 and Cy2 are each independently a bond, C₆₋₁₂ arylene, C₃₋₁₂ cycloalkylene, 5- to 12-membered heterocyclylene, or 5- to 12-membered heteroarylene, each of which is independently optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl.

In certain embodiments, Cy1 is 5- to 12-membered heterocyclylene optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl.

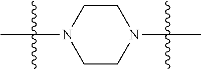 [SMILES: *N1CCN(*)CC1]

In certain embodiments, Cy1 is optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl. In certain embodiments, Cy1 is

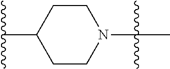 [SMILES: *C1CCN(*)CC1]

optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl. In certain embodiments, Cy1 is

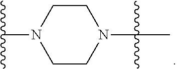 [SMILES: *N1CCN(*)CC1]

In certain embodiments, Cy1 is

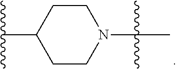 [SMILES: *C1CCN(*)CC1]

In certain embodiments, Cy1 is a bond. In certain embodiments, Cy1 is

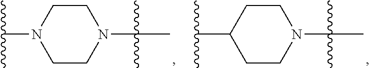 [SMILES: *N1CCN(*)CC1 ; *C1CCN(*)CC1]

or bond.

In certain embodiments, Cy2 is 5- to 12-membered heterocyclylene optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl.

In certain embodiments, Cy2 is

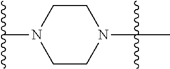 [SMILES: *N1CCN(*)CC1]

optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl. In certain embodiments, Cy2 is

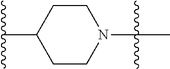 [SMILES: *C1CCN(*)CC1]

optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl. In certain embodiments, Cy2 is

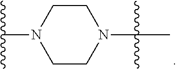 [SMILES: *N1CCN(*)CC1]

In certain embodiments, Cy2 is

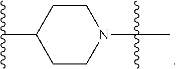 [SMILES: *C1CCN(*)CC1]

In certain embodiments, Cy2 is a bond. In certain embodiments, Cy2 is

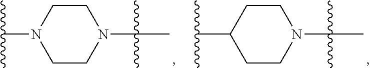 [SMILES: *N1CCN(*)CC1 ; *C1CCN(*)CC1]

or bond.

In certain embodiments, the linking moiety is of the Formula:

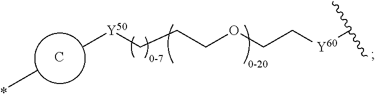

wherein
ring C is a 3- to 12-membered cycloalkylene or 3- to 12-membered heterocyclylene, each independently optionally substituted with one or more substituents independently selected from oxo, halo, C₁₋₄ alkyl, C₁₋₄ haloalkyl, C₁₋₄ alkoxy, or C₁₋₄ haloalkoxy;
each of Y⁵⁰ and Y⁶⁰ are independently a bond, —NR¹¹⁰—, —O—, —S(O)₀₋₂—, —NR¹¹⁰C(O)—, —C(O)NR¹¹⁰—, —NR¹¹⁰C(O)NR¹¹⁰—, —NR¹¹⁰S(O)₂—, —S(O)₂NR¹¹⁰—, —NR¹¹⁰S(O)₂NR¹¹⁰—, —CR¹²⁰═N—NR¹¹⁰—, —NR¹¹⁰—N═CR¹²⁰—, —C(O)—, —OC(O)—, —OC(O)O—, —(CH₂CH₂O)₁₋₅—, —C(O)O—, C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene, each independently optionally substituted with one or more substituents independently selected from oxo, halo, C₁₋₄ alkyl, C₁₋₄ haloalkyl, C₁₋₄ alkoxy, or C₁₋₄ haloalkoxy;
each R¹¹⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹²⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl; and
wherein the “*” and the wavy line represent a covalent bond.

In certain embodiments, each C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene of Y⁵⁰ and Y⁶⁰ is independently optionally substituted with one to five substituents independently selected from halo, C₁₋₄ alkyl, C₁₋₄ haloalkyl, C₁₋₄ alkoxy, or C₁₋₄ haloalkoxy.

In certain embodiments, the linking moiety is of the Formula:

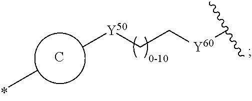

wherein
ring C is a 3- to 12-membered cycloalkylene or 3- to 12-membered heterocyclylene, each independently optionally substituted with one or more substituents independently selected from oxo, halo, C₁₋₄ alkyl, C₁₋₄ haloalkyl, C₁₋₄ alkoxy, or C₁₋₄ haloalkoxy;
each of Y⁵⁰ and Y⁶⁰ are independently a bond, —NR¹¹⁰—, —O—, —S(O)₀₋₂—, —NR¹¹⁰C(O)—, —C(O)NR¹¹⁰—, —NR¹¹⁰C(O)NR¹¹⁰—, —NR¹¹⁰S(O)₂—, —S(O)₂NR¹¹⁰—, —NR¹¹⁰S(O)₂NR¹¹⁰—, —CR¹²⁰═N—NR¹¹⁰—, —NR¹¹⁰—N═CR¹²⁰—, —C(O)—, —OC(O)—, —OC(O)O—, —(CH₂CH₂O)₁₋₅—, —C(O)O—, C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene, each independently optionally substituted with one or more substituents independently selected from oxo, halo, C₁₋₄ alkyl, C₁₋₄ haloalkyl, C₁₋₄ alkoxy, or C₁₋₄ haloalkoxy;
each R¹¹⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹²⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl; and
wherein the “*” and the wavy line represent a covalent bond.

In certain embodiments, the linking moiety is of the formula:

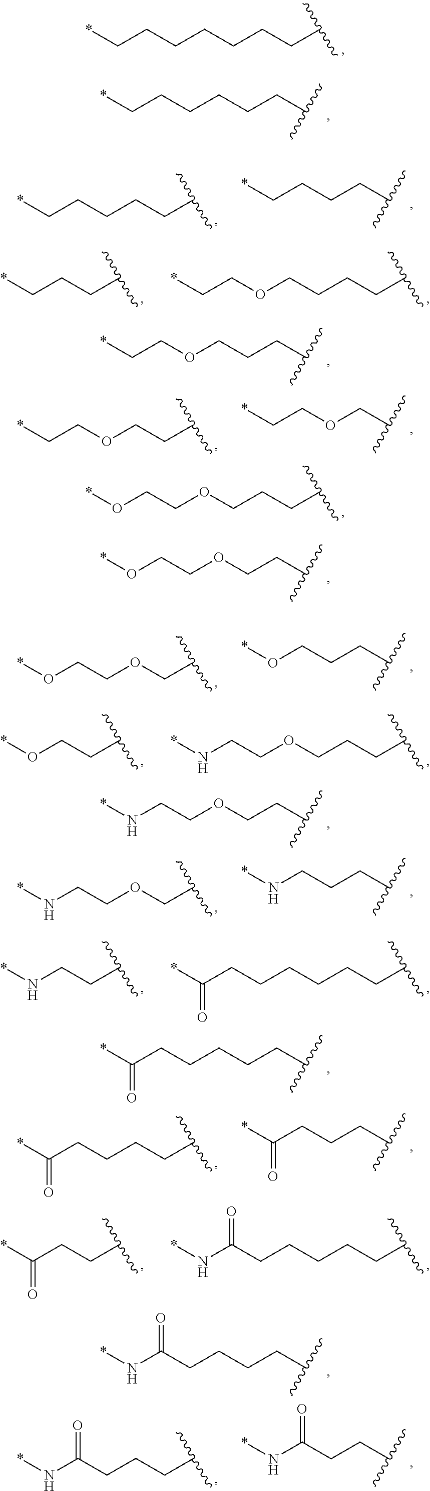

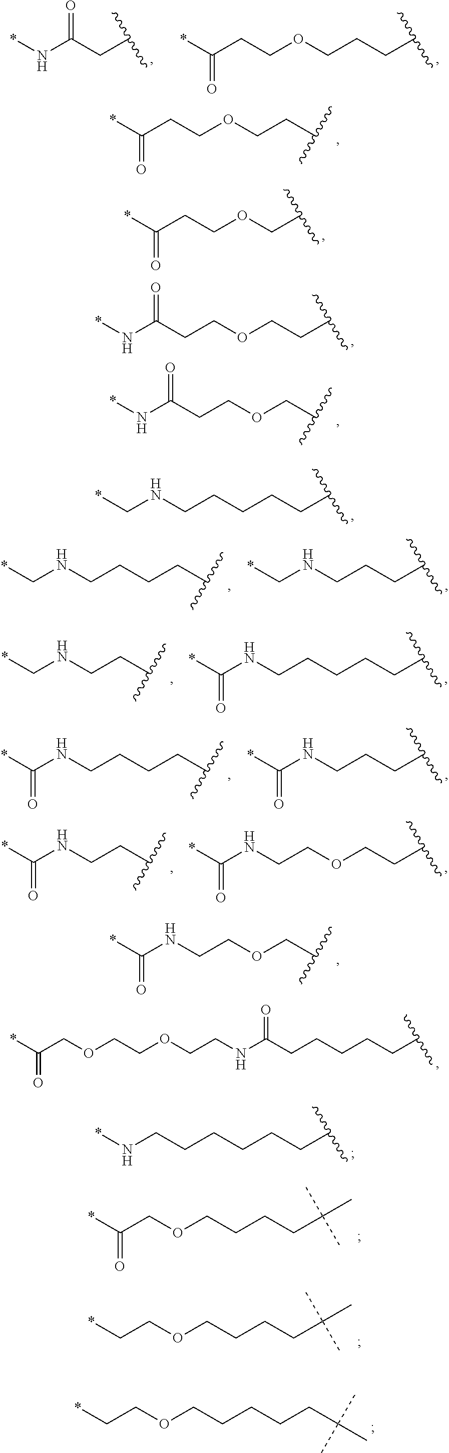

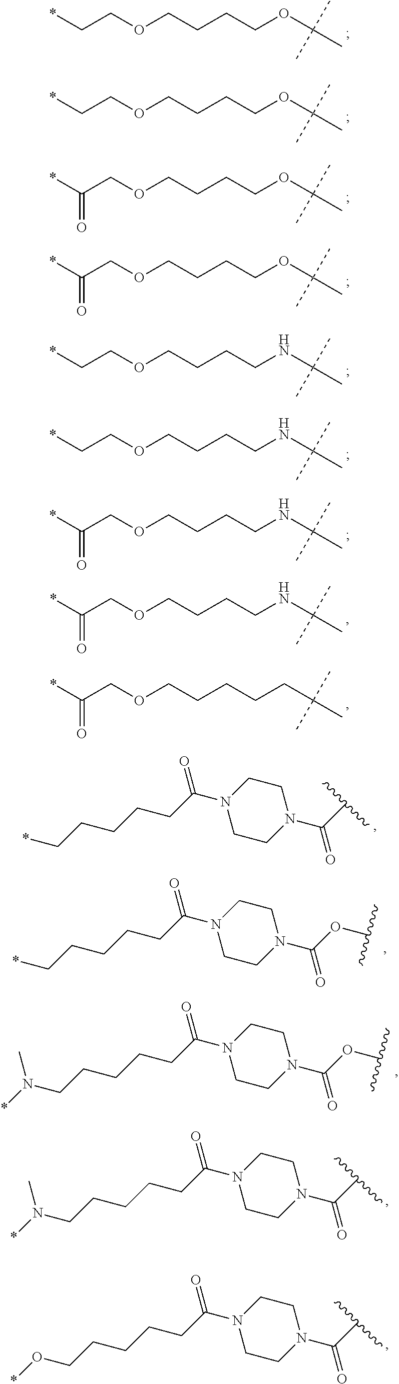

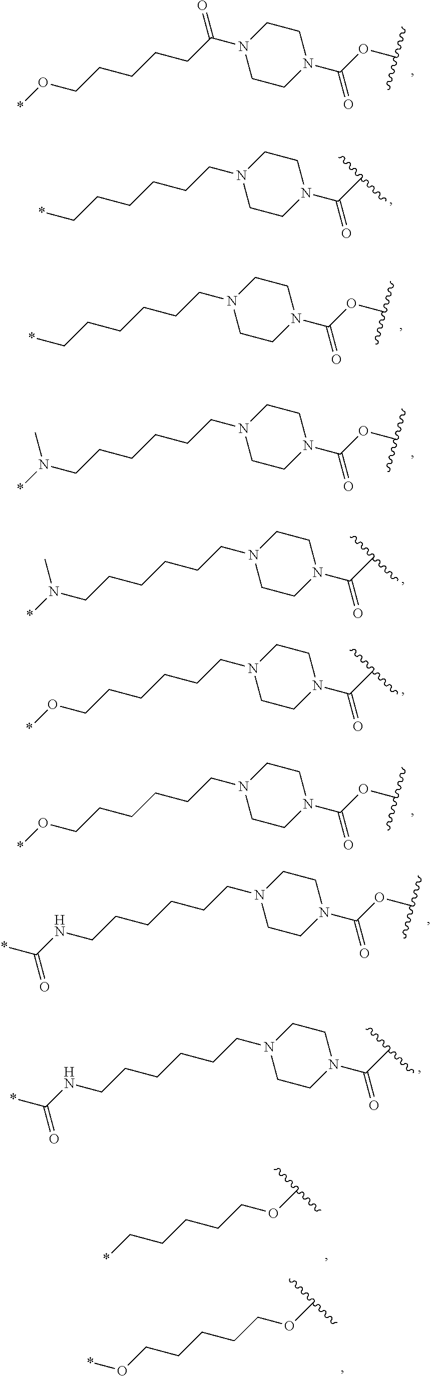

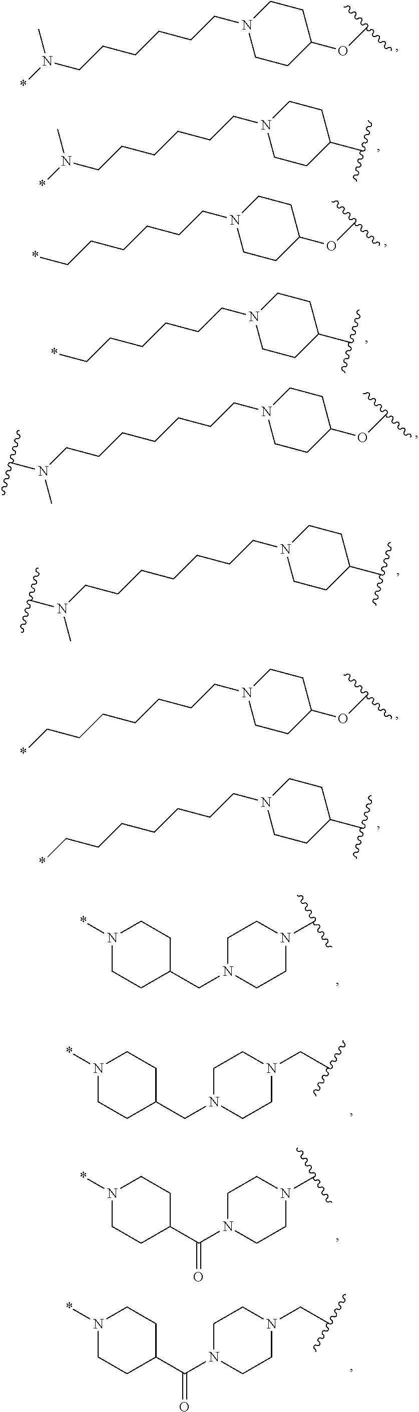

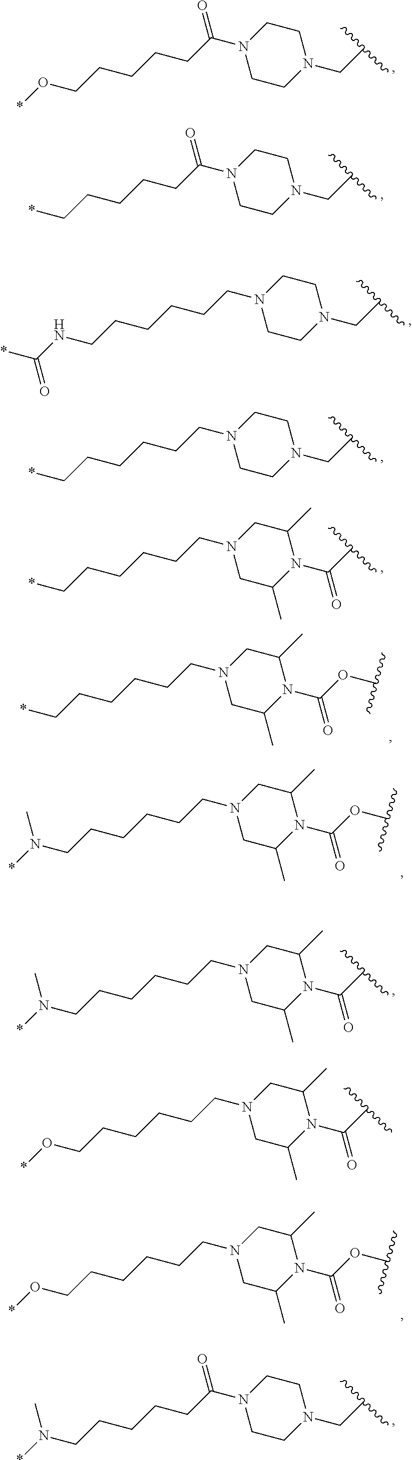

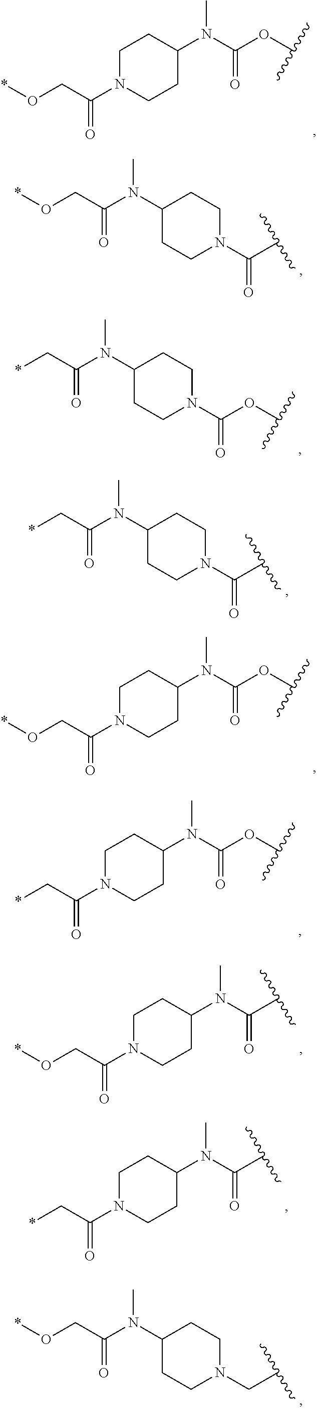

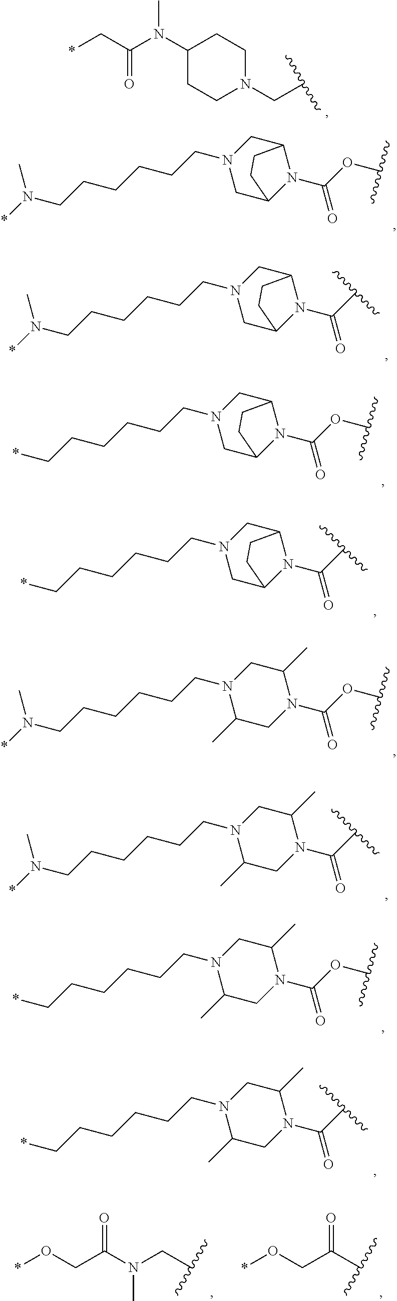

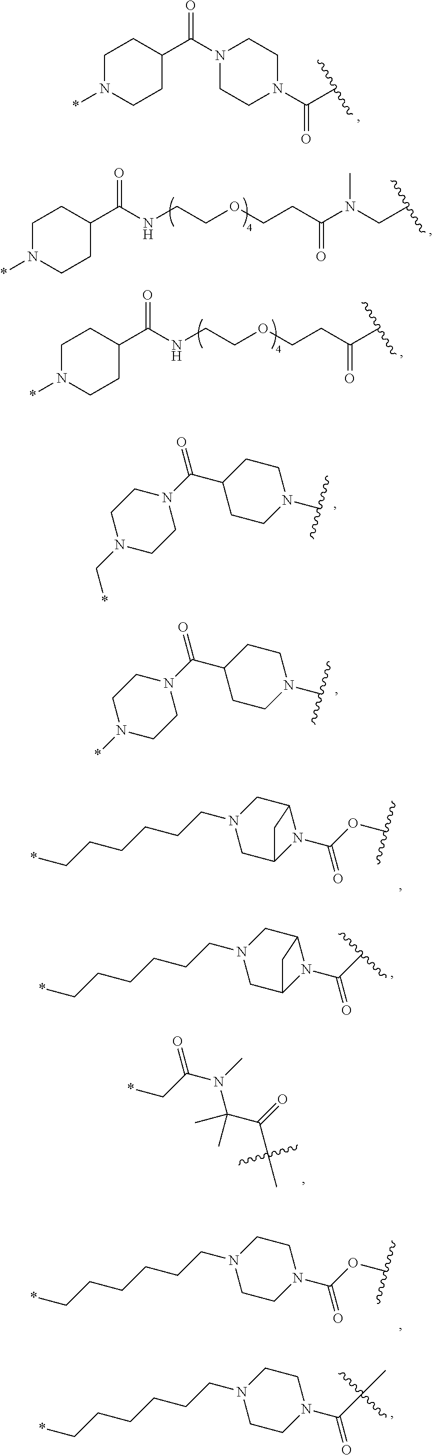

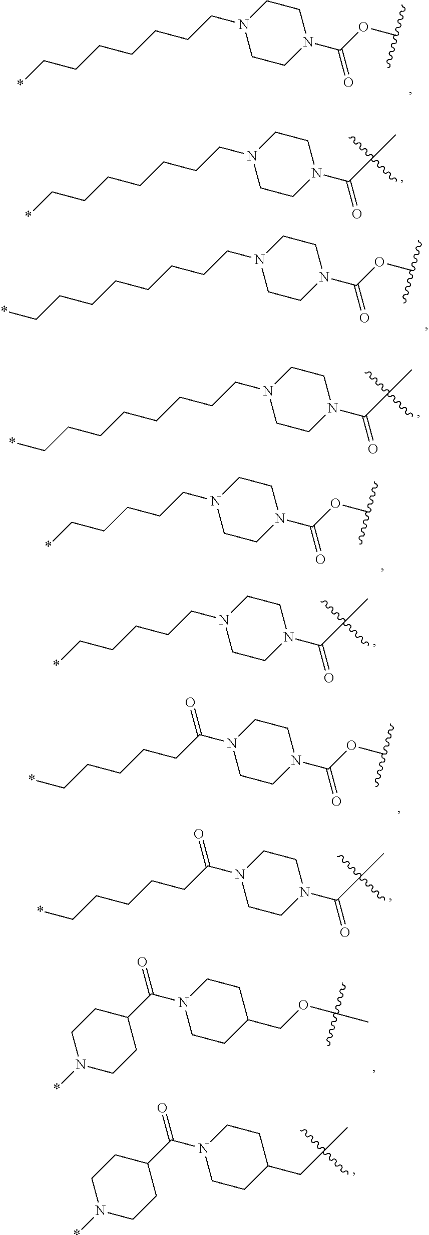

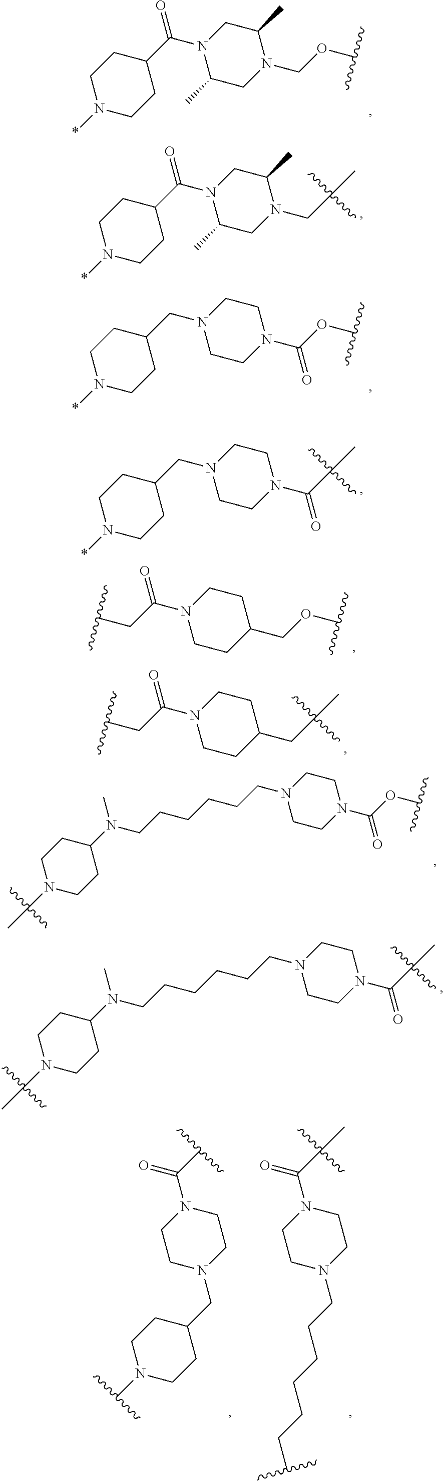

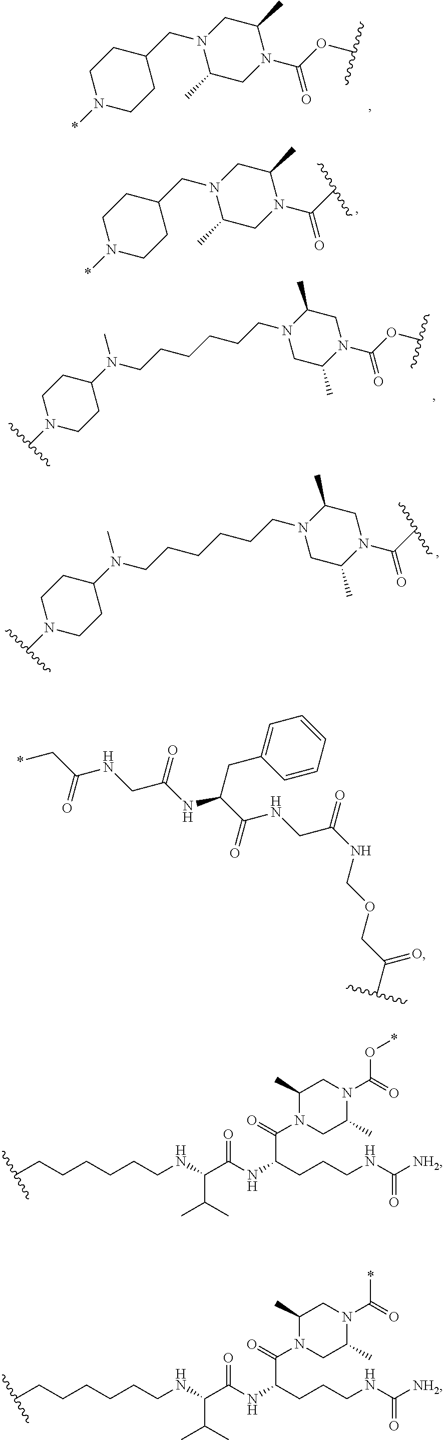

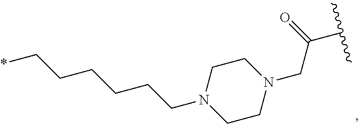 [SMILES: *CCCCCCN1CCN(CC(*)=O)CC1]

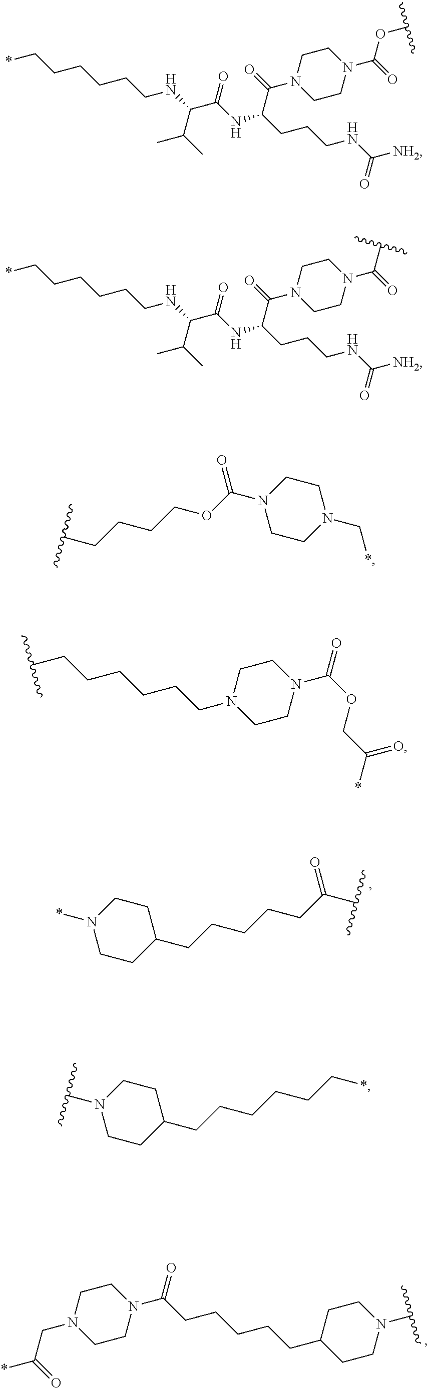

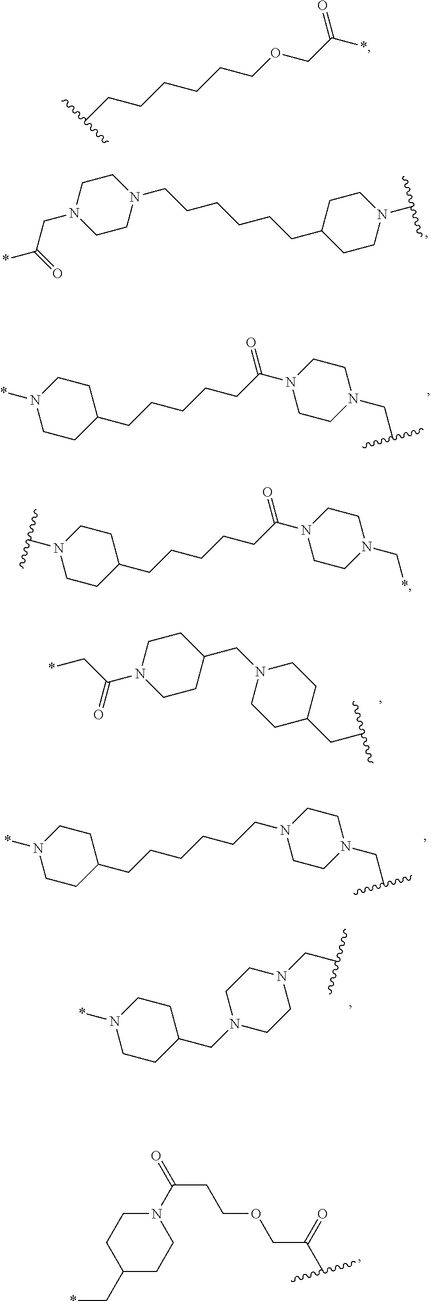

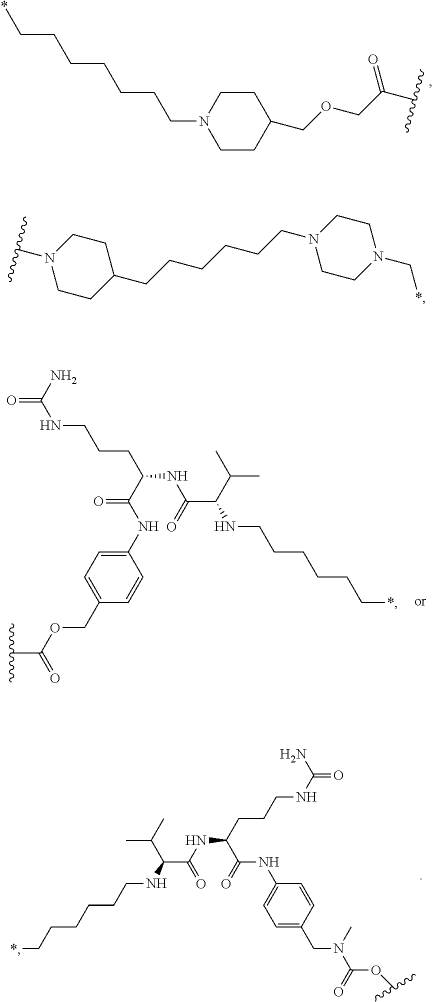

wherein the “*” and the wavy or dashed line represent a covalent bond. It is understood that either end can be connected to A¹.

In certain embodiments, the linking moiety is of the formula:

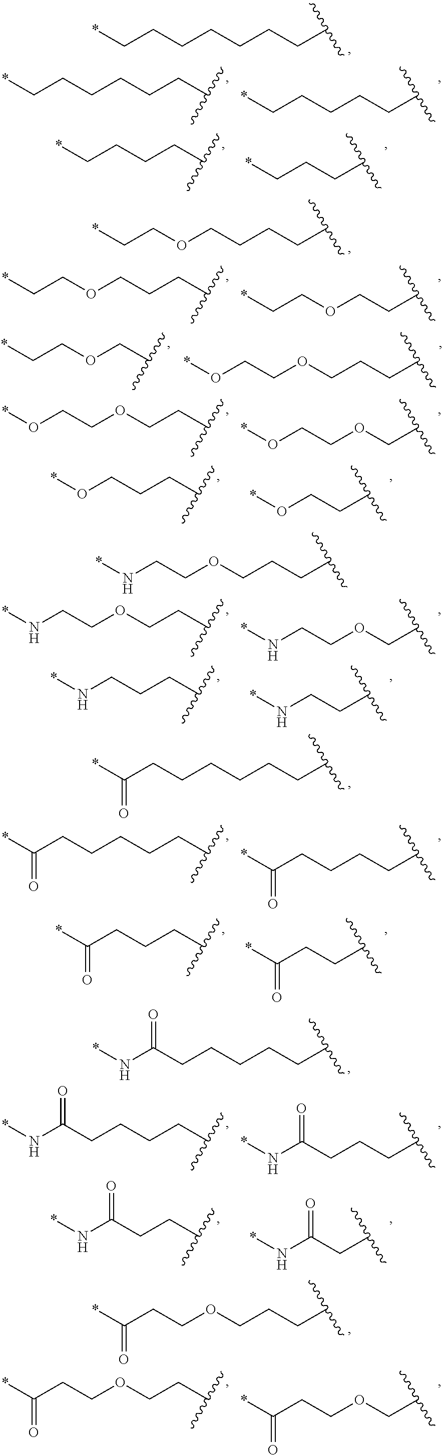

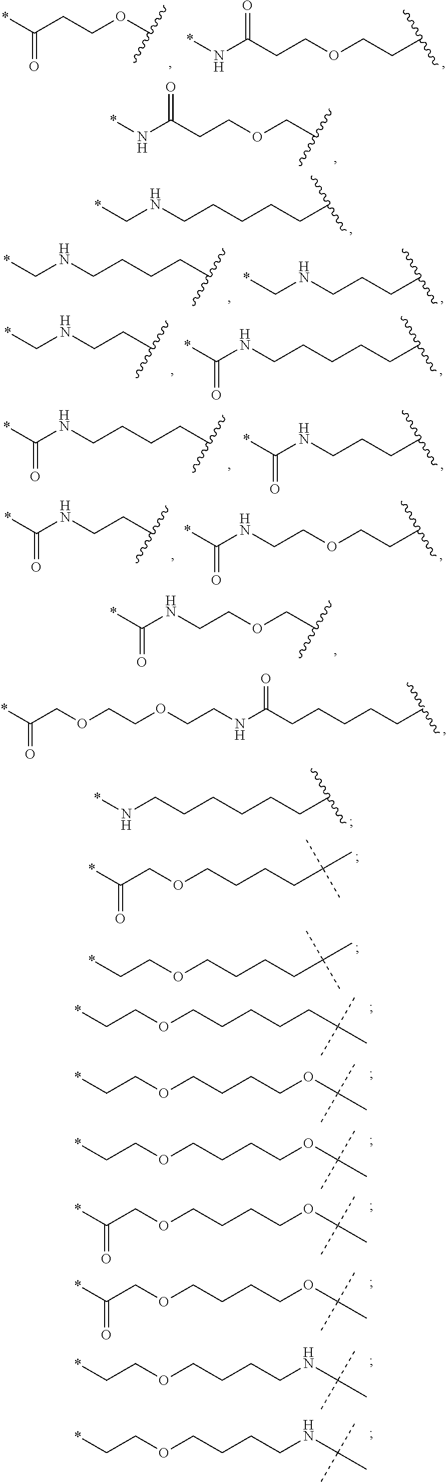

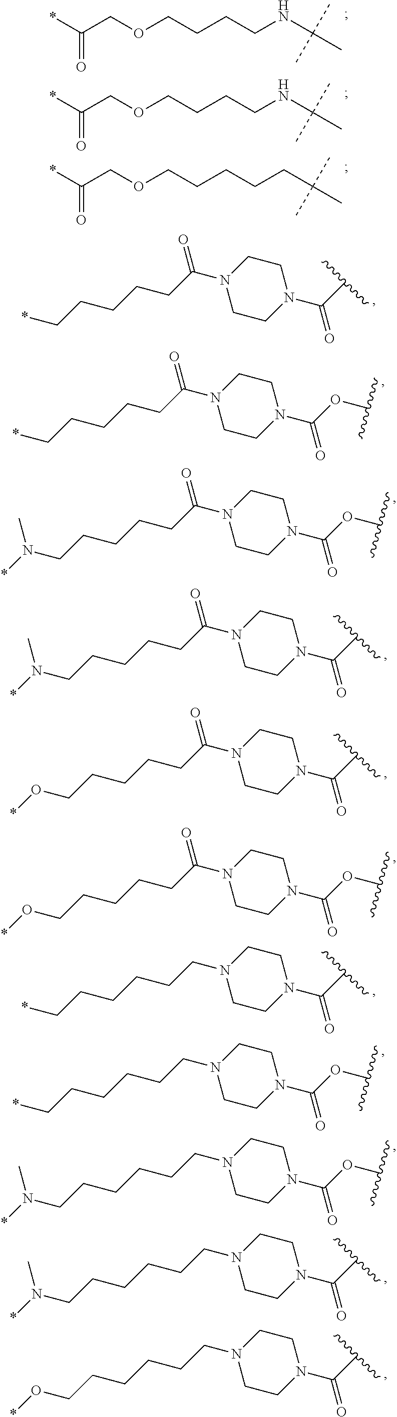

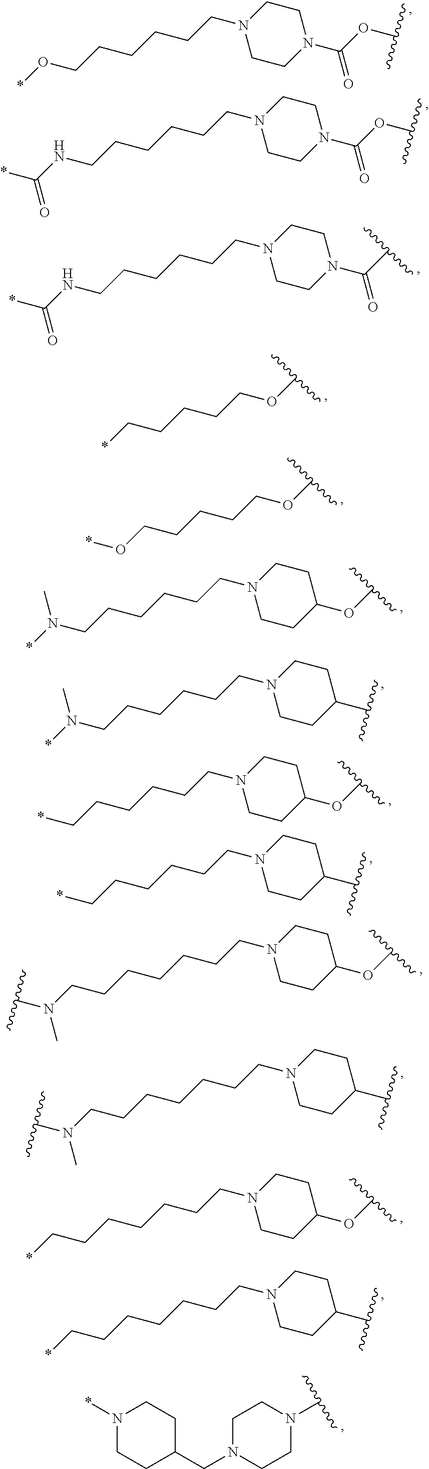

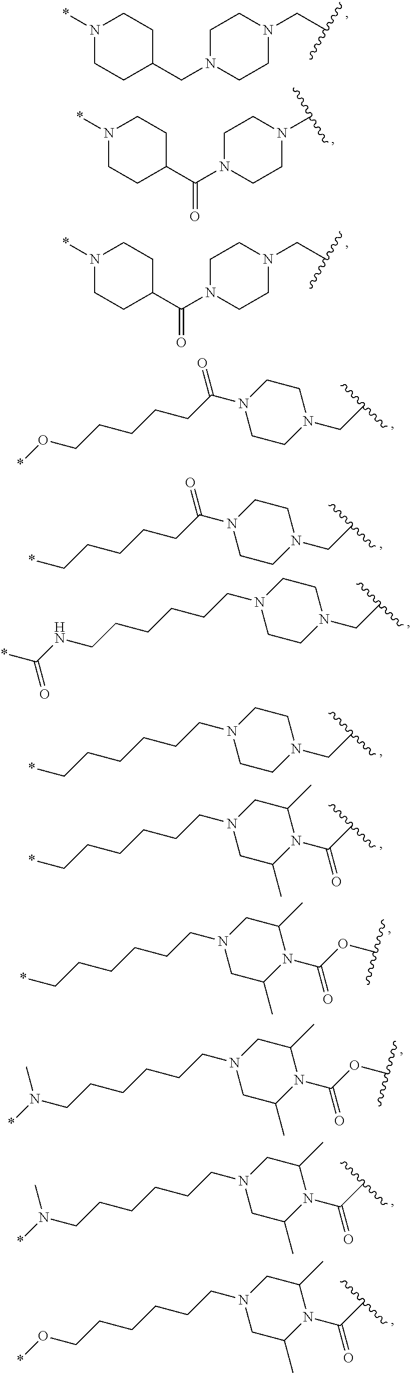

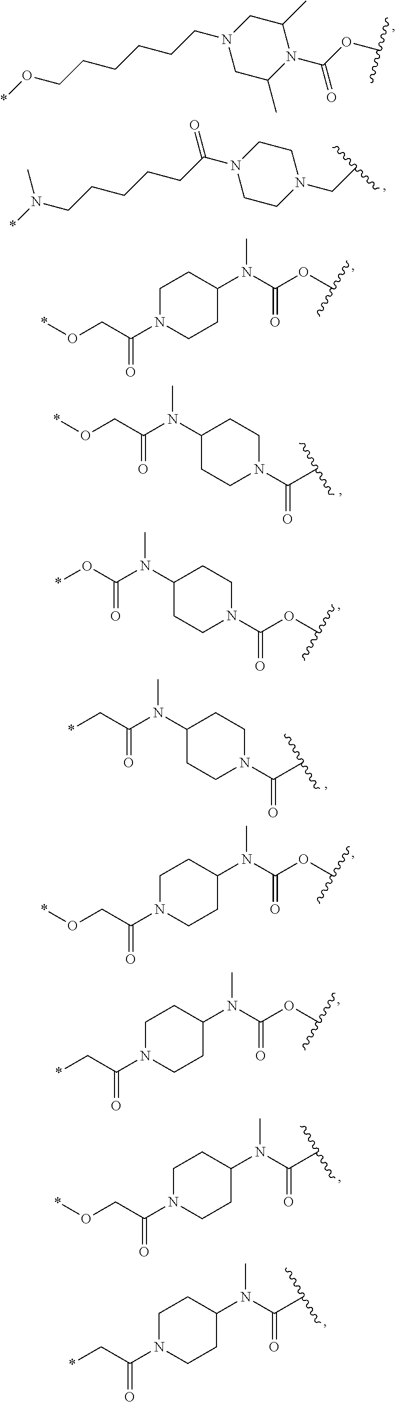

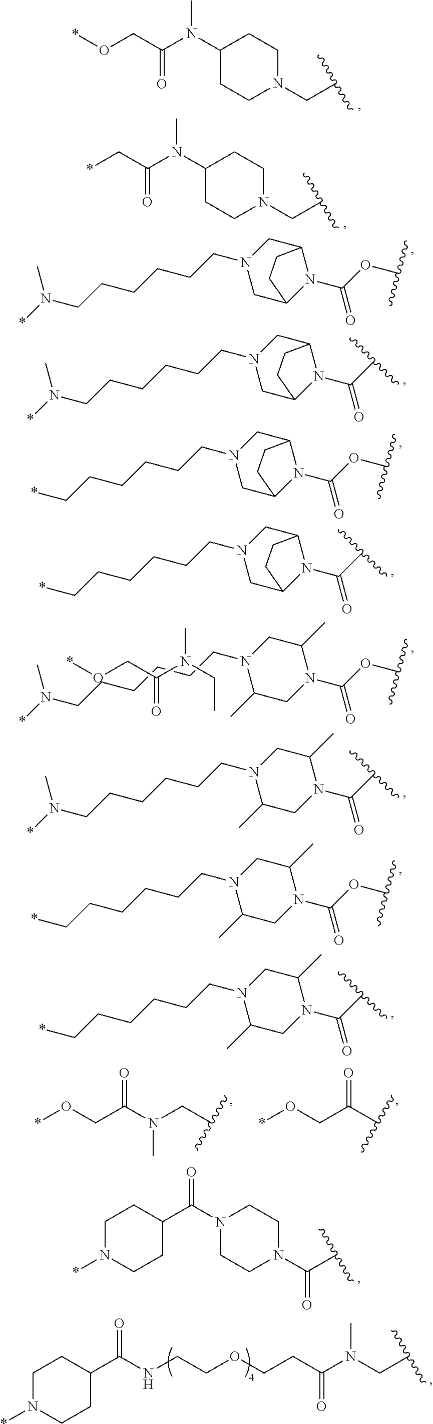

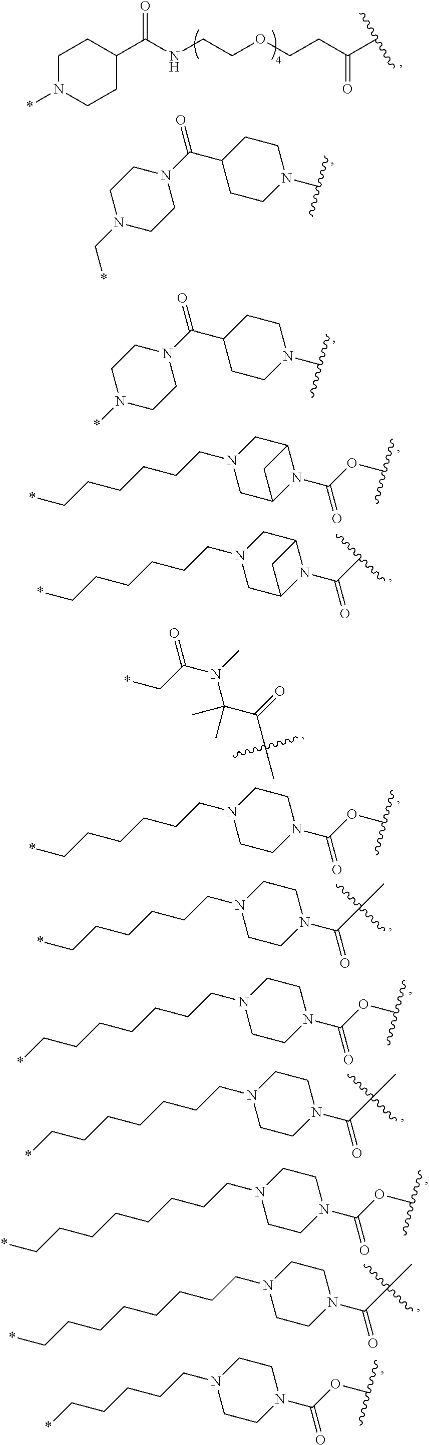

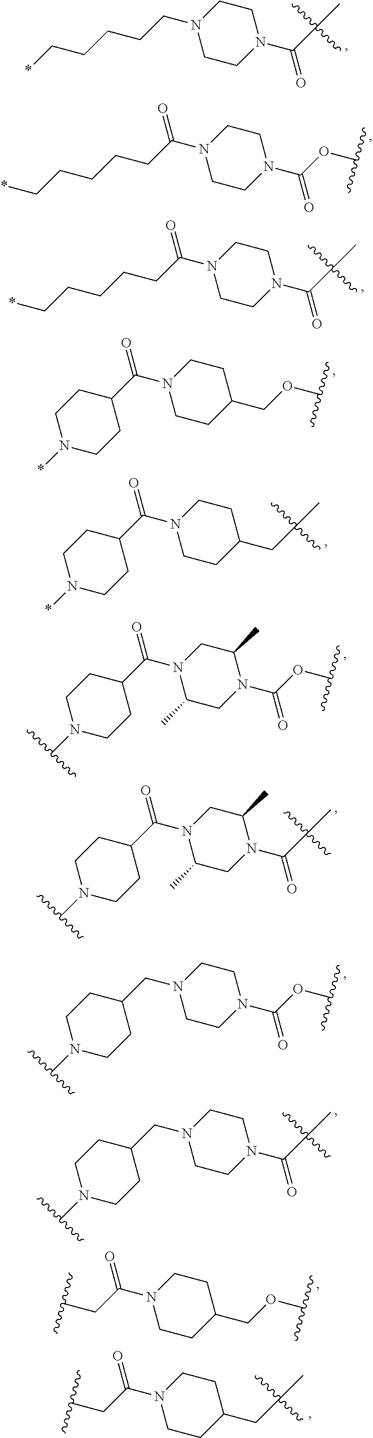

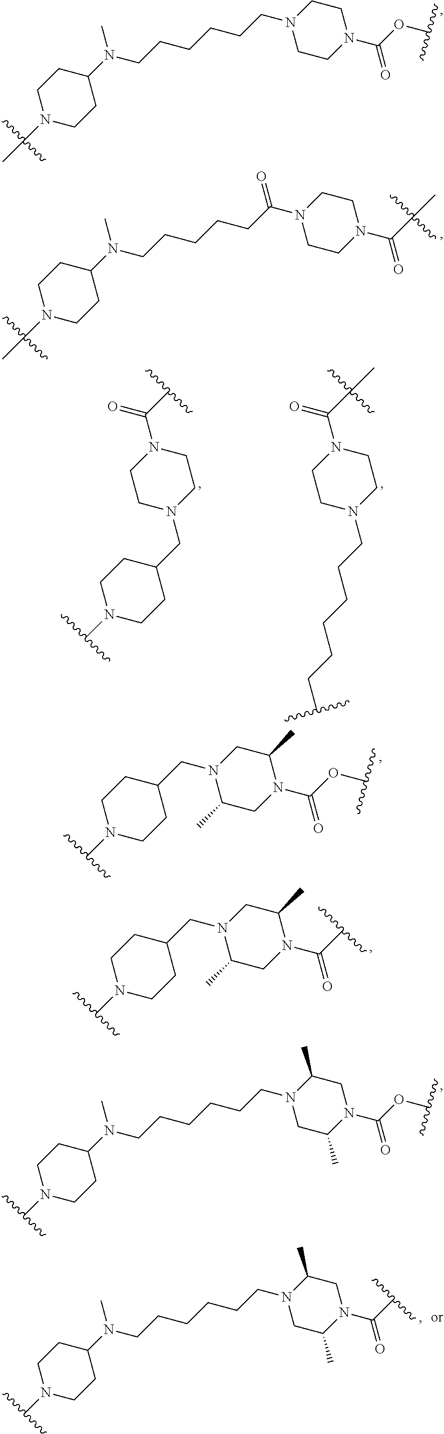

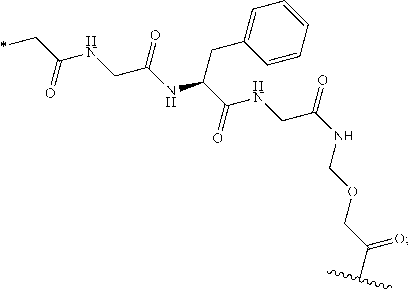 [SMILES: *CC(=O)NCC(=O)N[C@@H](Cc1ccccc1)C(=O)NCC(=O)NCOCC(*)=O]

wherein the “*” and the wavy or dashed line represent a covalent bond. It is understood that either * or the wavy or dashed line can be connected to A¹.

In certain embodiments, provided is a compound as in Table 1 or a stereoisomer, mixture of stereoisomers, hydrate, solvate, isotopically enriched analog, or pharmaceutically acceptable salt thereof.

TABLE 1

| Cpd | |
|---|---|
| No. | Structure |
| 1 | 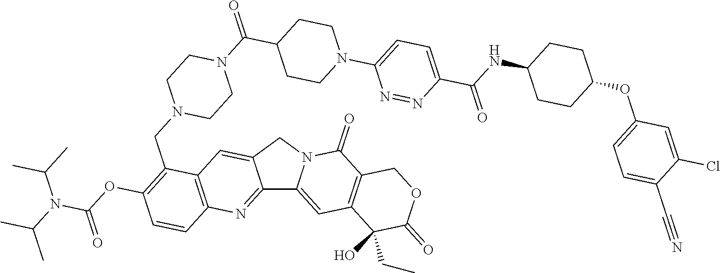 [SMILES: CC[C@@]1(O)C(=O)OCc2c1cc1n(c2=O)Cc2cc3c(CN4CCN(C(=O)C5CCN(c6ccc(C(=O)N[C@H]7CC[C@H](Oc8ccc(C#N)c(Cl)c8)CC7)nn6)CC5)CC4)c(OC(=O)N(C(C)C)C(C)C)ccc3nc2-1] |
| 2 | 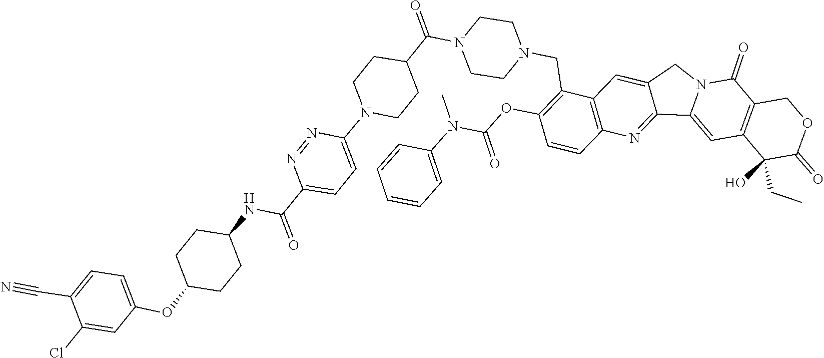 [SMILES: CC[C@@]1(O)C(=O)OCc2c1cc1n(c2=O)Cc2cc3c(CN4CCN(C(=O)C5CCN(c6ccc(C(=O)N[C@H]7CC[C@H](Oc8ccc(C#N)c(Cl)c8)CC7)nn6)CC5)CC4)c(OC(=O)N(C)c4ccccc4)ccc3nc2-1] |
| 3 | 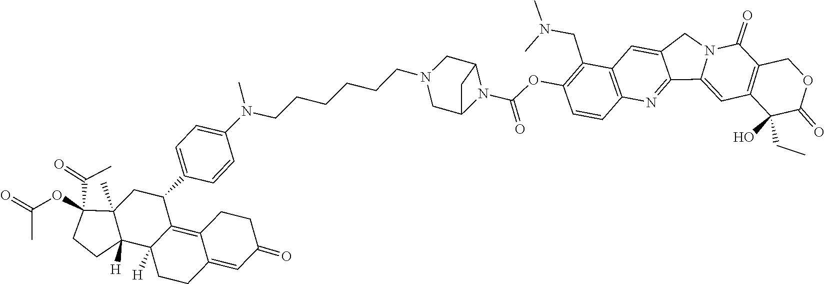 [SMILES: CC[C@@]1(O)C(=O)OCc2c1cc1n(c2=O)Cc2cc3c(CN(C)C)c(OC(=O)N4C5CC4CN(CCCCCCN(C)c4ccc([C@H]6C[C@@]7(C)[C@@H](CC[C@]7(OC(C)=O)C(C)=O)[C@@H]7CCC8=CC(=O)CCC8=C76)cc4)C5)ccc3nc2-1] |
| 4 | 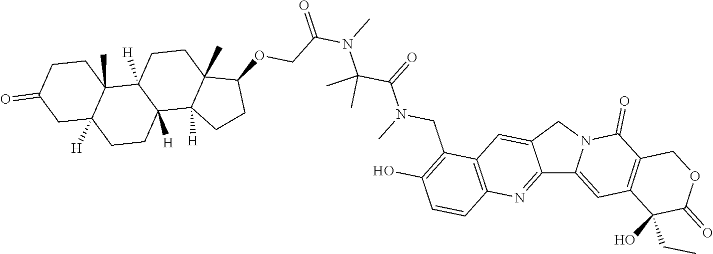 [SMILES: CC[C@@]1(O)C(=O)OCc2c1cc1n(c2=O)Cc2cc3c(CN(C)C(=O)C(C)(C)N(C)C(=O)CO[C@H]4CC[C@H]5[C@@H]6CC[C@H]7CC(=O)CC[C@]7(C)[C@H]6CC[C@]45C)c(O)ccc3nc2-1] |
| 5 | 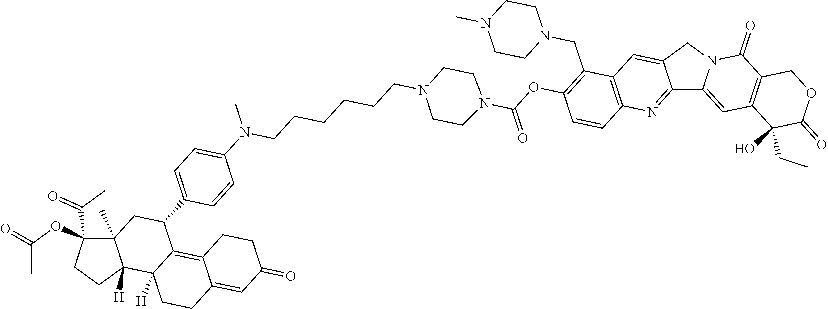 [SMILES: CC[C@@]1(O)C(=O)OCc2c1cc1n(c2=O)Cc2cc3c(CN4CCN(C)CC4)c(OC(=O)N4CCN(CCCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)ccc3nc2-1] |
| 6 | 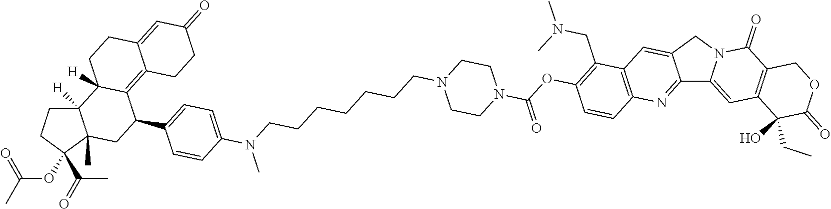 [SMILES: CC[C@@]1(O)C(=O)OCc2c1cc1n(c2=O)Cc2cc3c(CN(C)C)c(OC(=O)N4CCN(CCCCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)ccc3nc2-1] |
| 7 | 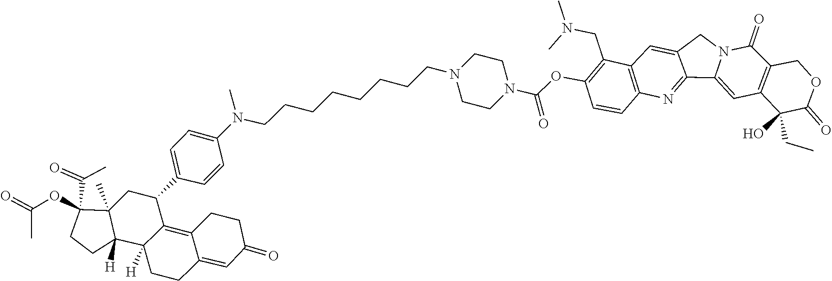 [SMILES: CC[C@@]1(O)C(=O)OCc2c1cc1n(c2=O)Cc2cc3c(CN(C)C)c(OC(=O)N4CCN(CCCCCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)ccc3nc2-1] |
| 8 | 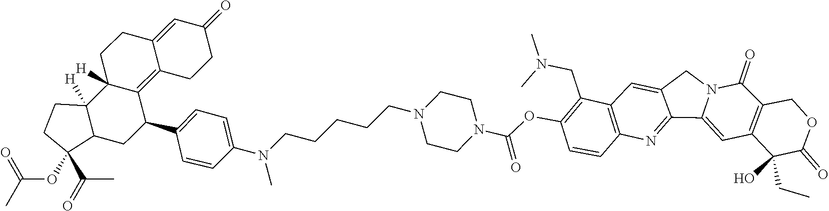 [SMILES: CC[C@@]1(O)C(=O)OCc2c1cc1n(c2=O)Cc2cc3c(CN(C)C)c(OC(=O)N4CCN(CCCCCN(C)c5ccc([C@H]6CC7[C@@H](CC[C@]7(OC(C)=O)C(C)=O)[C@@H]7CCC8=CC(=O)CCC8=C76)cc5)CC4)ccc3nc2-1] |
| 9 | 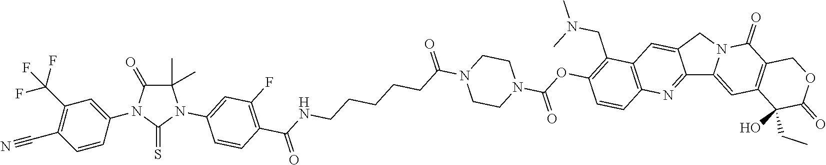 [SMILES: CC[C@@]1(O)C(=O)OCc2c1cc1n(c2=O)Cc2cc3c(CN(C)C)c(OC(=O)N4CCN(C(=O)CCCCCNC(=O)c5ccc(N6C(=S)N(c7ccc(C#N)c(C(F)(F)F)c7)C(=O)C6(C)C)cc5F)CC4)ccc3nc2-1] |
| 10 | 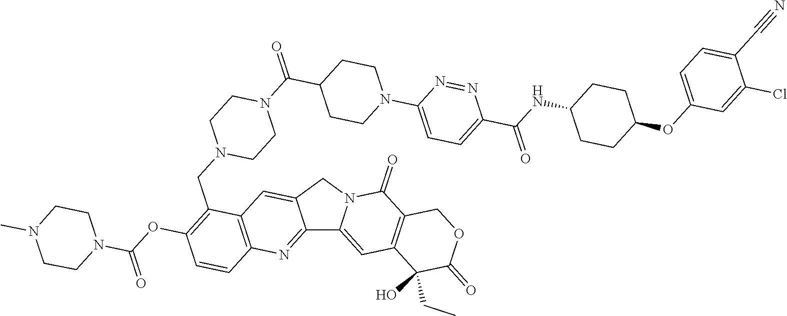 [SMILES: CC[C@@]1(O)C(=O)OCc2c1cc1n(c2=O)Cc2cc3c(CN4CCN(C(=O)C5CCN(c6ccc(C(=O)N[C@H]7CC[C@H](Oc8ccc(C#N)c(Cl)c8)CC7)nn6)CC5)CC4)c(OC(=O)N4CCN(C)CC4)ccc3nc2-1] |
| 11 | 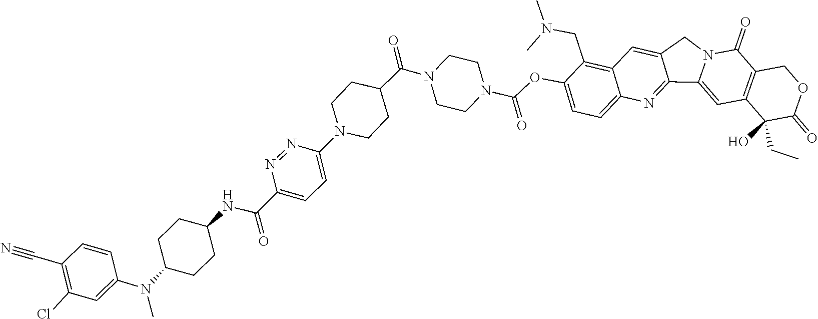 [SMILES: CC[C@@]1(O)C(=O)OCc2c1cc1n(c2=O)Cc2cc3c(CN(C)C)c(OC(=O)N4CCN(C(=O)C5CCN(c6ccc(C(=O)N[C@H]7CC[C@H](N(C)c8ccc(C#N)c(Cl)c8)CC7)nn6)CC5)CC4)ccc3nc2-1] |
| 12 | 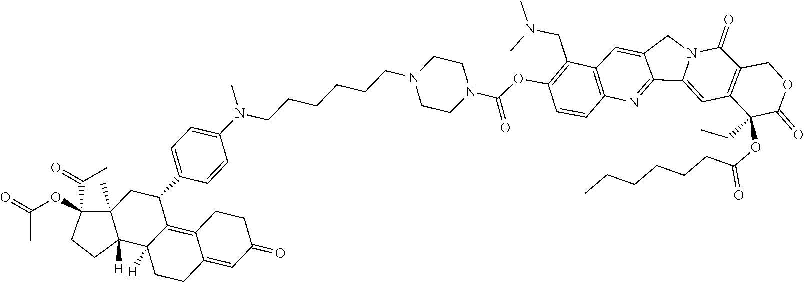 [SMILES: CCCCCCC(=O)O[C@]1(CC)C(=O)OCc2c1cc1n(c2=O)Cc2cc3c(CN(C)C)c(OC(=O)N4CCN(CCCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)ccc3nc2-1] |
| 13 | 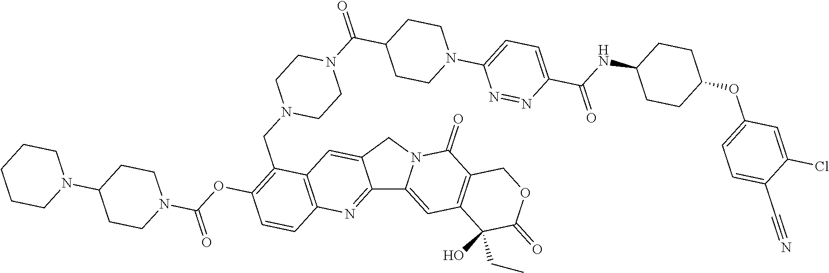 [SMILES: CC[C@@]1(O)C(=O)OCc2c1cc1n(c2=O)Cc2cc3c(CN4CCN(C(=O)C5CCN(c6ccc(C(=O)N[C@H]7CC[C@H](Oc8ccc(C#N)c(Cl)c8)CC7)nn6)CC5)CC4)c(OC(=O)N4CCC(N5CCCCC5)CC4)ccc3nc2-1] |
| 14 | 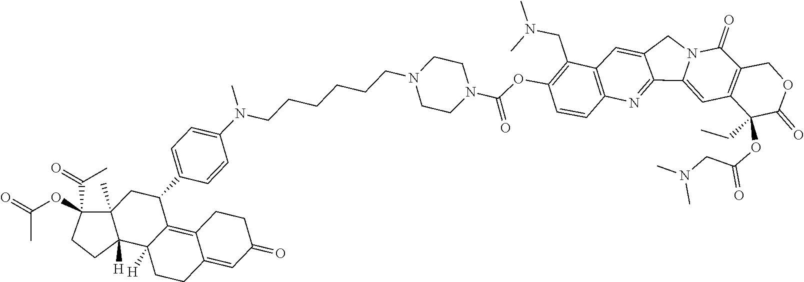 [SMILES: CC[C@@]1(OC(=O)CN(C)C)C(=O)OCc2c1cc1n(c2=O)Cc2cc3c(CN(C)C)c(OC(=O)N4CCN(CCCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)ccc3nc2-1] |
| 15 | 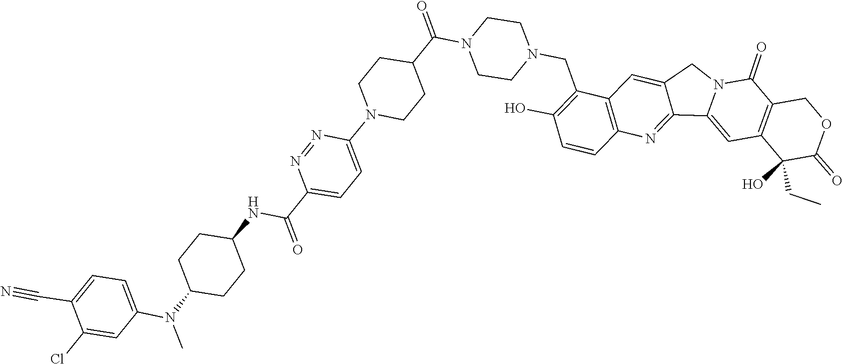 [SMILES: CC[C@@]1(O)C(=O)OCc2c1cc1n(c2=O)Cc2cc3c(CN4CCN(C(=O)C5CCN(c6ccc(C(=O)N[C@H]7CC[C@H](N(C)c8ccc(C#N)c(Cl)c8)CC7)nn6)CC5)CC4)c(O)ccc3nc2-1] |
| 16 | 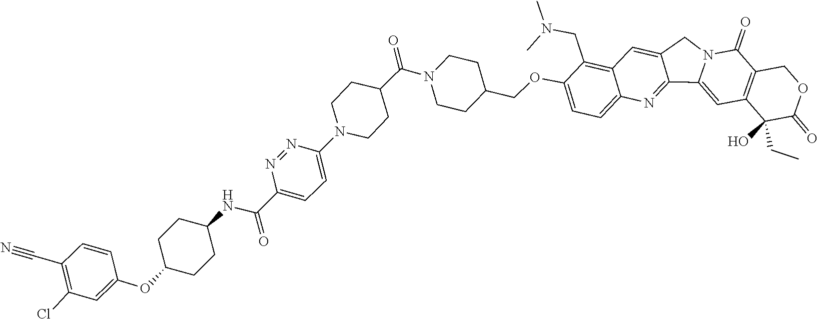 [SMILES: CC[C@@]1(O)C(=O)OCc2c1cc1n(c2=O)Cc2cc3c(CN(C)C)c(OCC4CCN(C(=O)C5CCN(c6ccc(C(=O)N[C@H]7CC[C@H](Oc8ccc(C#N)c(Cl)c8)CC7)nn6)CC5)CC4)ccc3nc2-1] |
| 17 | 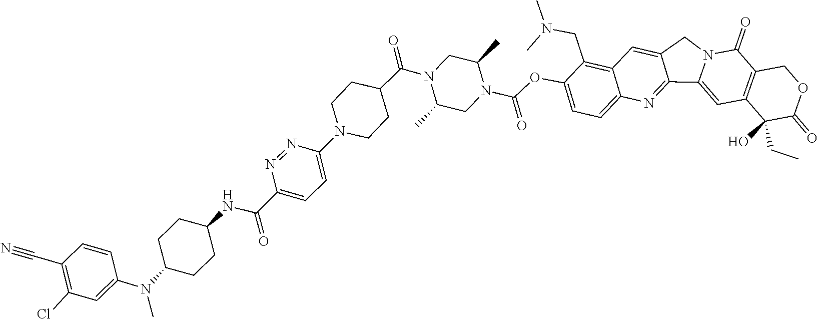 [SMILES: CC[C@@]1(O)C(=O)OCc2c1cc1n(c2=O)Cc2cc3c(CN(C)C)c(OC(=O)N4C[C@H](C)N(C(=O)C5CCN(c6ccc(C(=O)N[C@H]7CC[C@H](N(C)c8ccc(C#N)c(Cl)c8)CC7)nn6)CC5)C[C@H]4C)ccc3nc2-1] |
| 18 | 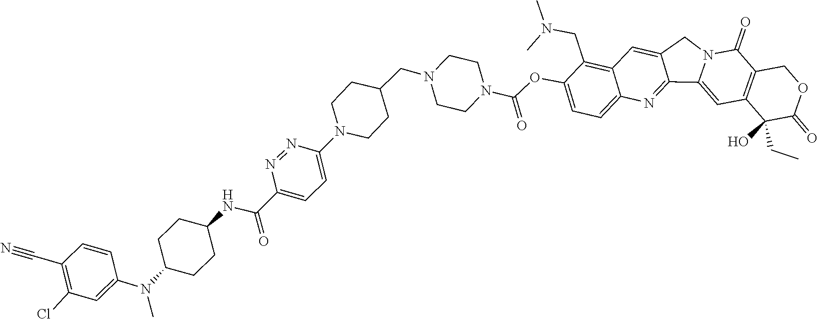 [SMILES: CC[C@@]1(O)C(=O)OCc2c1cc1n(c2=O)Cc2cc3c(CN(C)C)c(OC(=O)N4CCN(CC5CCN(c6ccc(C(=O)N[C@H]7CC[C@H](N(C)c8ccc(C#N)c(Cl)c8)CC7)nn6)CC5)CC4)ccc3nc2-1] |
| 19 | 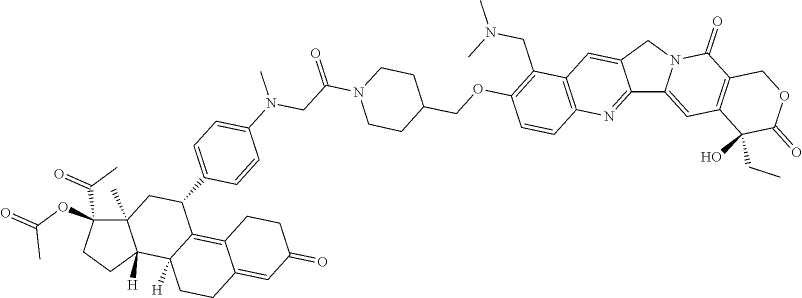 [SMILES: CC[C@@]1(O)C(=O)OCc2c1cc1n(c2=O)Cc2cc3c(CN(C)C)c(OCC4CCN(C(=O)CN(C)c5ccc([C@H]6C[C@@]7(C)[C@@H](CC[C@]7(OC(C)=O)C(C)=O)[C@@H]7CCC8=CC(=O)CCC8=C76)cc5)CC4)ccc3nc2-1] |
| 20 | 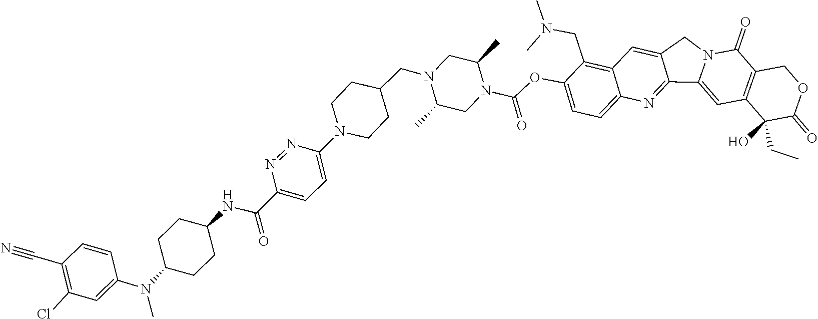 [SMILES: CC[C@@]1(O)C(=O)OCc2c1cc1n(c2=O)Cc2cc3c(CN(C)C)c(OC(=O)N4C[C@H](C)N(CC5CCN(c6ccc(C(=O)N[C@H]7CC[C@H](N(C)c8ccc(C#N)c(Cl)c8)CC7)nn6)CC5)C[C@H]4C)ccc3nc2-1] |
| 21 | 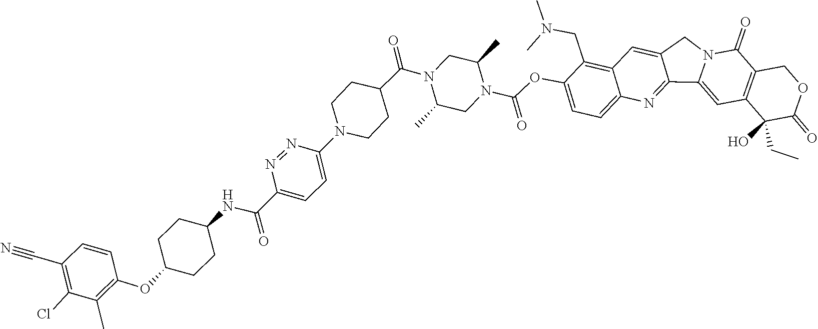 [SMILES: CC[C@@]1(O)C(=O)OCc2c1cc1n(c2=O)Cc2cc3c(CN(C)C)c(OC(=O)N4C[C@H](C)N(C(=O)C5CCN(c6ccc(C(=O)N[C@H]7CC[C@H](Oc8ccc(C#N)c(Cl)c8C)CC7)nn6)CC5)C[C@H]4C)ccc3nc2-1] |
| 22 | 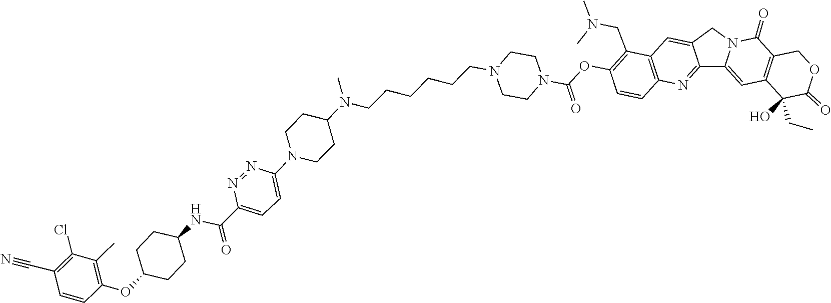 [SMILES: CC[C@@]1(O)C(=O)OCc2c1cc1n(c2=O)Cc2cc3c(CN(C)C)c(OC(=O)N4CCN(CCCCCCN(C)C5CCN(c6ccc(C(=O)N[C@H]7CC[C@H](Oc8ccc(C#N)c(Cl)c8C)CC7)nn6)CC5)CC4)ccc3nc2-1] |
| 23 | 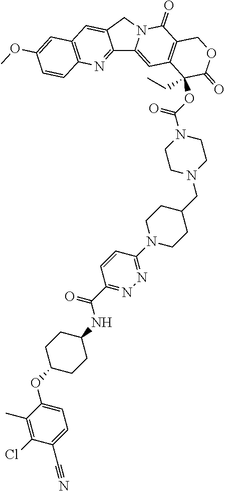 [SMILES: CC[C@@]1(OC(=O)N2CCN(CC3CCN(c4ccc(C(=O)N[C@H]5CC[C@H](Oc6ccc(C#N)c(Cl)c6C)CC5)nn4)CC3)CC2)C(=O)OCc2c1cc1n(c2=O)Cc2cc3cc(OC)ccc3nc2-1] |
| 24 | 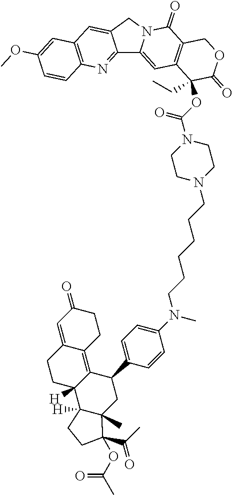 [SMILES: CC[C@@]1(OC(=O)N2CCN(CCCCCCN(C)c3ccc([C@H]4C[C@@]5(C)[C@@H](CC[C@]5(OC(C)=O)C(C)=O)[C@@H]5CCC6=CC(=O)CCC6=C54)cc3)CC2)C(=O)OCc2c1cc1n(c2=O)Cc2cc3cc(OC)ccc3nc2-1] |
| 25 | 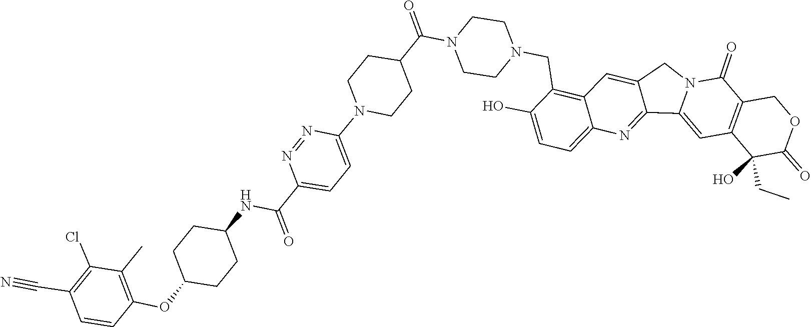 [SMILES: CC[C@@]1(O)C(=O)OCc2c1cc1n(c2=O)Cc2cc3c(CN4CCN(C(=O)C5CCN(c6ccc(C(=O)N[C@H]7CC[C@H](Oc8ccc(C#N)c(Cl)c8C)CC7)nn6)CC5)CC4)c(O)ccc3nc2-1] |
| 26 | 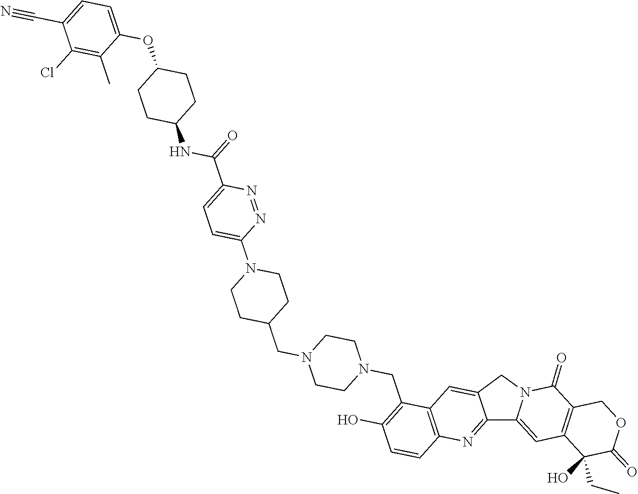 [SMILES: CC[C@@]1(O)C(=O)OCc2c1cc1n(c2=O)Cc2cc3c(CN4CCN(CC5CCN(c6ccc(C(=O)N[C@H]7CC[C@H](Oc8ccc(C#N)c(Cl)c8C)CC7)nn6)CC5)CC4)c(O)ccc3nc2-1] |
| 27 | 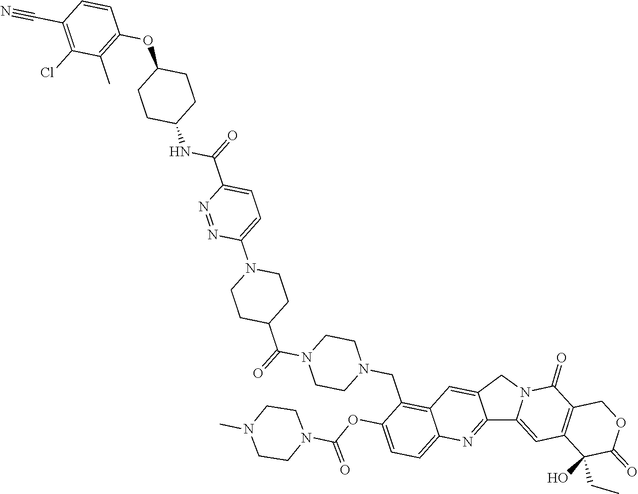 [SMILES: CC[C@@]1(O)C(=O)OCc2c1cc1n(c2=O)Cc2cc3c(CN4CCN(C(=O)C5CCN(c6ccc(C(=O)N[C@H]7CC[C@H](Oc8ccc(C#N)c(Cl)c8C)CC7)nn6)CC5)CC4)c(OC(=O)N4CCN(C)CC4)ccc3nc2-1] |
| 28 | 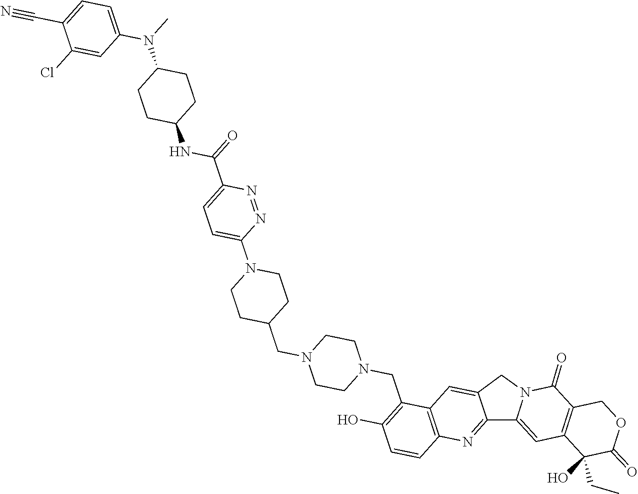 [SMILES: CC[C@@]1(O)C(=O)OCc2c1cc1n(c2=O)Cc2cc3c(CN4CCN(CC5CCN(c6ccc(C(=O)N[C@H]7CC[C@H](N(C)c8ccc(C#N)c(Cl)c8)CC7)nn6)CC5)CC4)c(O)ccc3nc2-1] |
| 29 | 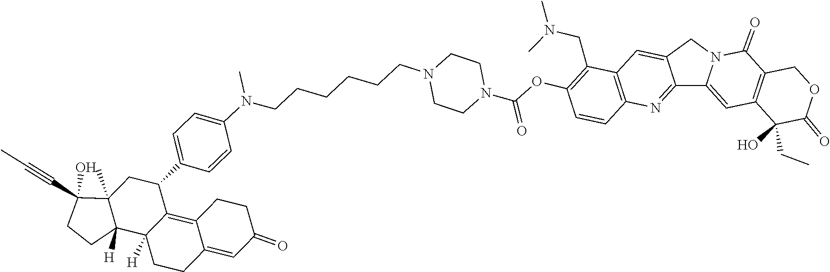 [SMILES: CC#C[C@]1(O)CC[C@H]2[C@@H]3CCC4=CC(=O)CCC4=C3[C@@H](c3ccc(N(C)CCCCCCN4CCN(C(=O)Oc5ccc6nc7c(cc6c5CN(C)C)Cn5c-7cc6c(c5=O)COC(=O)[C@]6(O)CC)CC4)cc3)C[C@@]21C] |
| 30 | 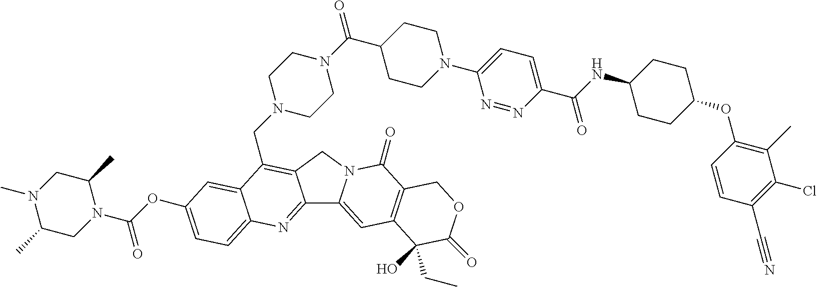 [SMILES: CC[C@@]1(O)C(=O)OCc2c1cc1n(c2=O)Cc2c-1nc1ccc(OC(=O)N3C[C@H](C)N(C)C[C@H]3C)cc1c2CN1CCN(C(=O)C2CCN(c3ccc(C(=O)N[C@H]4CC[C@H](Oc5ccc(C#N)c(Cl)c5C)CC4)nn3)CC2)CC1] |
| 31 | 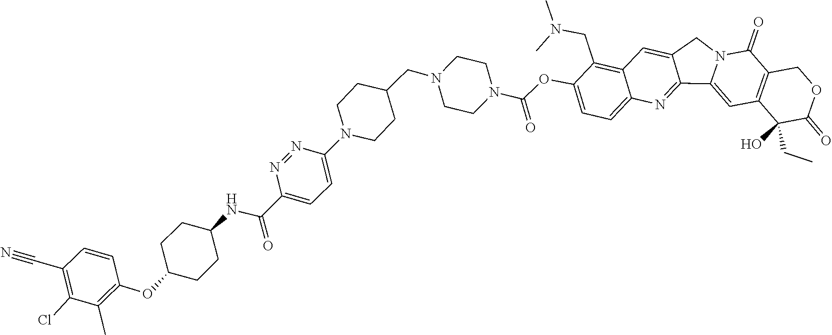 [SMILES: CC[C@@]1(O)C(=O)OCc2c1cc1n(c2=O)Cc2cc3c(CN(C)C)c(OC(=O)N4CCN(CC5CCN(c6ccc(C(=O)N[C@H]7CC[C@H](Oc8ccc(C#N)c(Cl)c8C)CC7)nn6)CC5)CC4)ccc3nc2-1] |
| 32 | 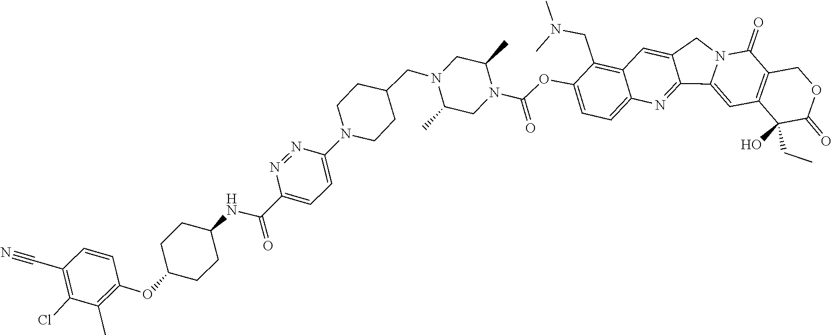 [SMILES: CC[C@@]1(O)C(=O)OCc2c1cc1n(c2=O)Cc2cc3c(CN(C)C)c(OC(=O)N4C[C@H](C)N(CC5CCN(c6ccc(C(=O)N[C@H]7CC[C@H](Oc8ccc(C#N)c(Cl)c8C)CC7)nn6)CC5)C[C@H]4C)ccc3nc2-1] |
| 33 | 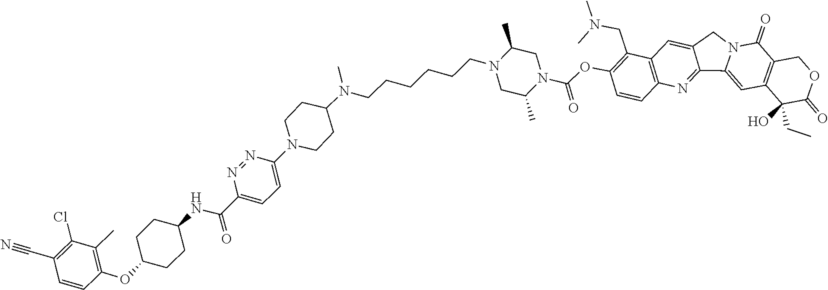 [SMILES: CC[C@@]1(O)C(=O)OCc2c1cc1n(c2=O)Cc2cc3c(CN(C)C)c(OC(=O)N4C[C@H](C)N(CCCCCCN(C)C5CCN(c6ccc(C(=O)N[C@H]7CC[C@H](Oc8ccc(C#N)c(Cl)c8C)CC7)nn6)CC5)C[C@H]4C)ccc3nc2-1] |
| 34 | 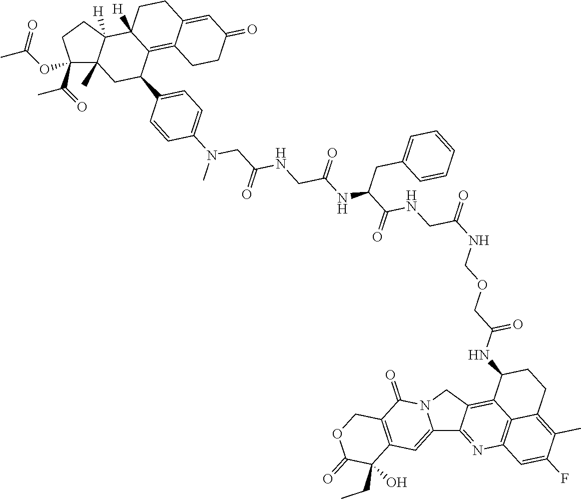 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CN(C)c1ccc([C@H]2C[C@@]4(C)[C@@H](CC[C@]4(OC(C)=O)C(C)=O)[C@@H]4CCC5=CC(=O)CCC5=C42)cc1)CC3] |
| 35 | 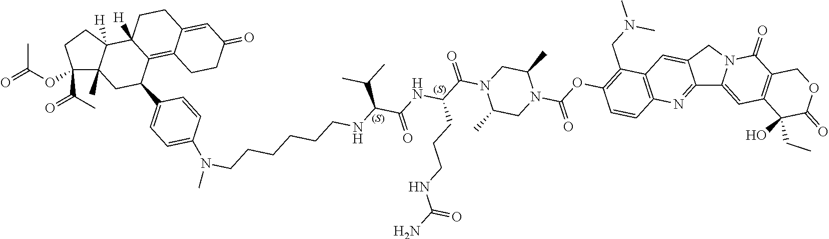 |
| 36 | 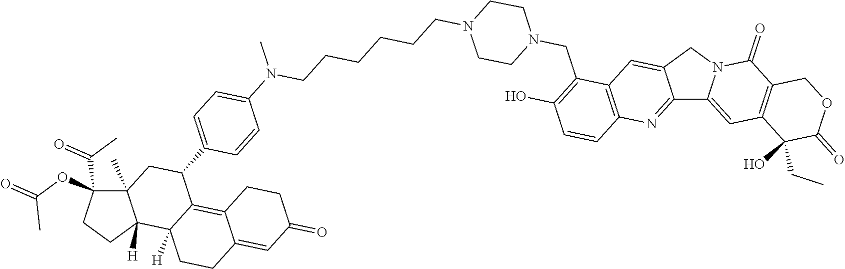 [SMILES: CC[C@@]1(O)C(=O)OCc2c1cc1n(c2=O)Cc2cc3c(CN4CCN(CCCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)c(O)ccc3nc2-1] |
| 37 | 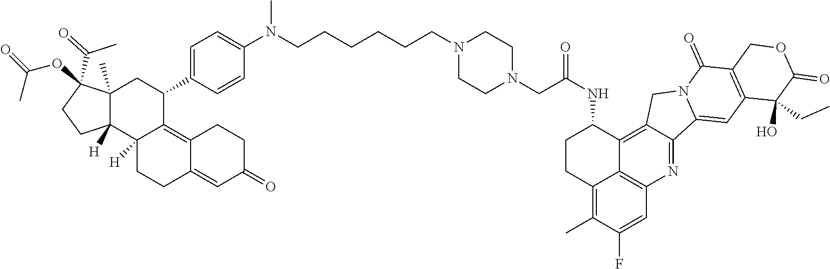 [SMILES: CC[C@@]1(O)C(=O)OCc2c1cc1n(c2=O)Cc2c-1nc1cc(F)c(C)c3c1c2[C@@H](NC(=O)CN1CCN(CCCCCCN(C)c2ccc([C@H]4C[C@@]5(C)[C@@H](CC[C@]5(OC(C)=O)C(C)=O)[C@@H]5CCC6=CC(=O)CCC6=C54)cc2)CC1)CC3] |
| 38 | 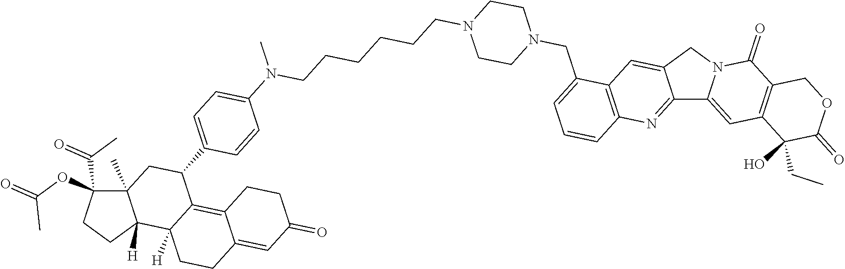 [SMILES: CC[C@@]1(O)C(=O)OCc2c1cc1n(c2=O)Cc2cc3c(CN4CCN(CCCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)cccc3nc2-1] |
| 39 | 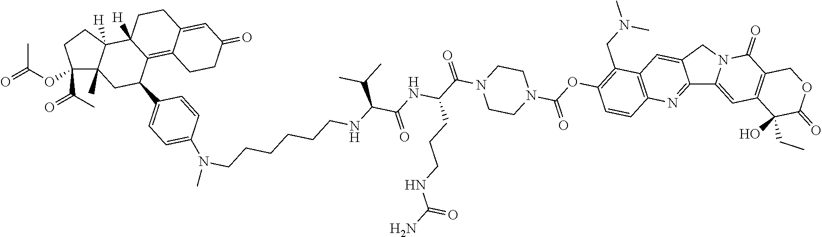 [SMILES: CC[C@@]1(O)C(=O)OCc2c1cc1n(c2=O)Cc2cc3c(CN(C)C)c(OC(=O)N4CCN(C(=O)[C@H](CCCNC(N)=O)NC(=O)[C@@H](NCCCCCCN(C)c5ccc([C@H]6C[C@@]7(C)[C@@H](CC[C@]7(OC(C)=O)C(C)=O)[C@@H]7CCC8=CC(=O)CCC8=C76)cc5)C(C)C)CC4)ccc3nc2-1] |
| 40 | 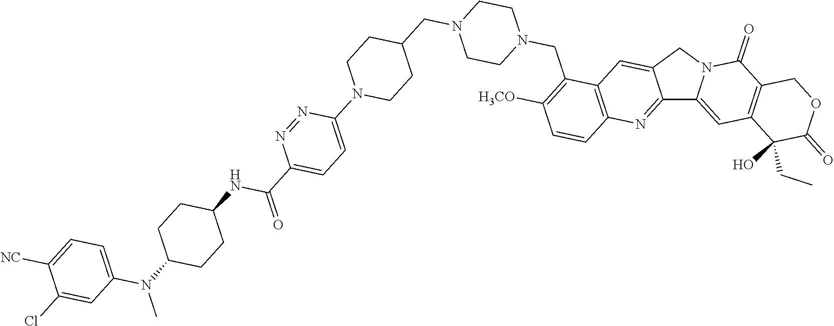 [SMILES: CC[C@@]1(O)C(=O)OCc2c1cc1n(c2=O)Cc2cc3c(CN4CCN(CC5CCN(c6ccc(C(=O)N[C@H]7CC[C@H](N(C)c8ccc(C#N)c(Cl)c8)CC7)nn6)CC5)CC4)c(OC)ccc3nc2-1] |
| 41 | 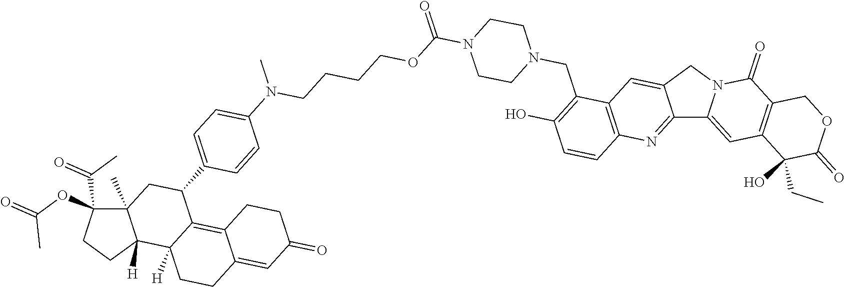 [SMILES: CC[C@@]1(O)C(=O)OCc2c1cc1n(c2=O)Cc2cc3c(CN4CCN(C(=O)OCCCCN(C)c5ccc([C@H]6C[C@@]7(C)[C@@H](CC[C@]7(OC(C)=O)C(C)=O)[C@@H]7CCC8=CC(=O)CCC8=C76)cc5)CC4)c(O)ccc3nc2-1] |
| 42 | 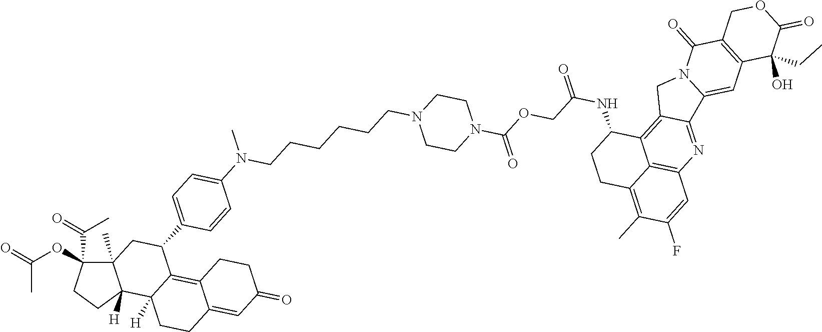 [SMILES: CC[C@@]1(O)C(=O)OCc2c1cc1n(c2=O)Cc2c-1nc1cc(F)c(C)c3c1c2[C@@H](NC(=O)COC(=O)N1CCN(CCCCCCN(C)c2ccc([C@H]4C[C@@]5(C)[C@@H](CC[C@]5(OC(C)=O)C(C)=O)[C@@H]5CCC6=CC(=O)CCC6=C54)cc2)CC1)CC3] |
| 43 | 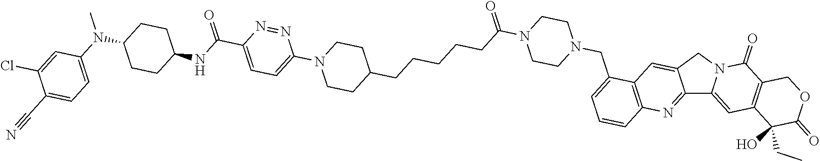 [SMILES: CC[C@@]1(O)C(=O)OCc2c1cc1n(c2=O)Cc2cc3c(CN4CCN(C(=O)CCCCCC5CCN(c6ccc(C(=O)N[C@H]7CC[C@H](N(C)c8ccc(C#N)c(Cl)c8)CC7)nn6)CC5)CC4)cccc3nc2-1] |
| 44 | 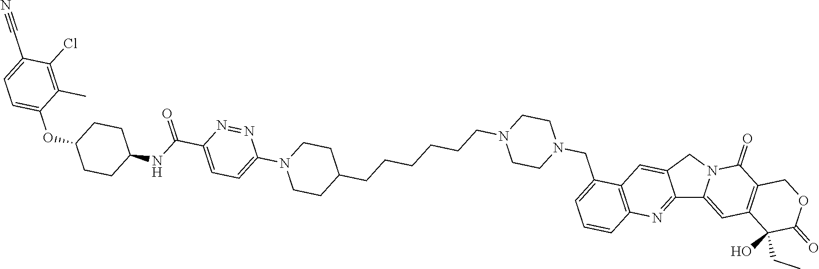 [SMILES: CC[C@@]1(O)C(=O)OCc2c1cc1n(c2=O)Cc2cc3c(CN4CCN(CCCCCCC5CCN(c6ccc(C(=O)N[C@H]7CC[C@H](Oc8ccc(C#N)c(Cl)c8C)CC7)nn6)CC5)CC4)cccc3nc2-1] |
| 45 | 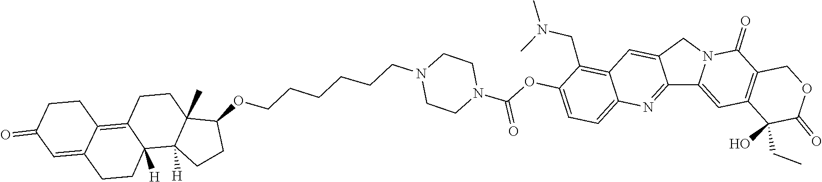 [SMILES: CC[C@@]1(O)C(=O)OCc2c1cc1n(c2=O)Cc2cc3c(CN(C)C)c(OC(=O)N4CCN(CCCCCCO[C@H]5CC[C@H]6[C@@H]7CCC8=CC(=O)CCC8=C7CC[C@]56C)CC4)ccc3nc2-1] |
| 46 | 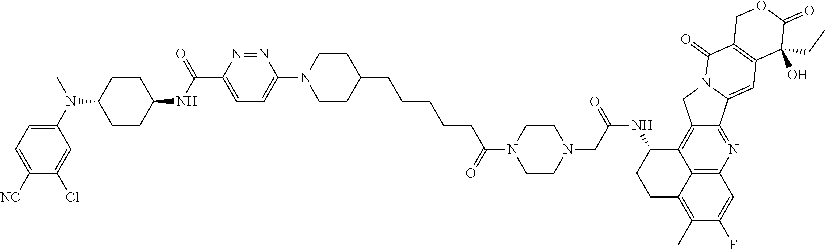 [SMILES: CC[C@@]1(O)C(=O)OCc2c1cc1n(c2=O)Cc2c-1nc1cc(F)c(C)c3c1c2[C@@H](NC(=O)CN1CCN(C(=O)CCCCCC2CCN(c4ccc(C(=O)N[C@H]5CC[C@H](N(C)c6ccc(C#N)c(Cl)c6)CC5)nn4)CC2)CC1)CC3] |
| 47 | 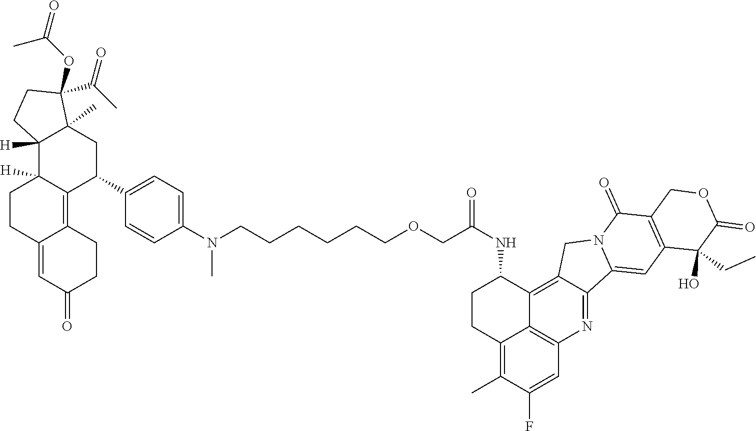 [SMILES: CC[C@@]1(O)C(=O)OCc2c1cc1n(c2=O)Cc2c-1nc1cc(F)c(C)c3c1c2[C@@H](NC(=O)COCCCCCCN(C)c1ccc([C@H]2C[C@@]4(C)[C@@H](CC[C@]4(OC(C)=O)C(C)=O)[C@@H]4CCC5=CC(=O)CCC5=C42)cc1)CC3] |
| 48 | 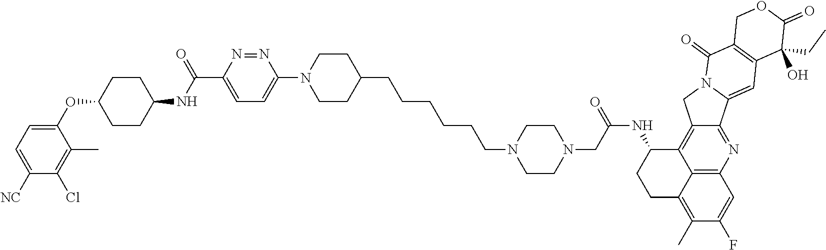 [SMILES: CC[C@@]1(O)C(=O)OCc2c1cc1n(c2=O)Cc2c-1nc1cc(F)c(C)c3c1c2[C@@H](NC(=O)CN1CCN(CCCCCCC2CCN(c4ccc(C(=O)N[C@H]5CC[C@H](Oc6ccc(C#N)c(Cl)c6C)CC5)nn4)CC2)CC1)CC3] |
| 49 | 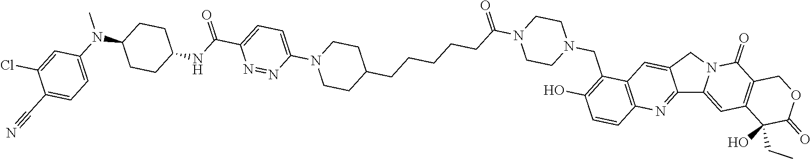 [SMILES: CC[C@@]1(O)C(=O)OCc2c1cc1n(c2=O)Cc2cc3c(CN4CCN(C(=O)CCCCCC5CCN(c6ccc(C(=O)N[C@H]7CC[C@H](N(C)c8ccc(C#N)c(Cl)c8)CC7)nn6)CC5)CC4)c(O)ccc3nc2-1] |
| 50 | 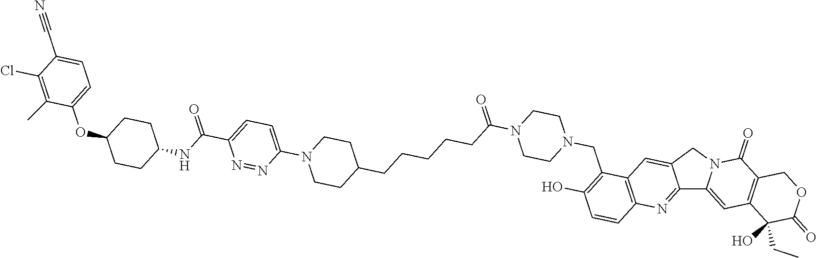 [SMILES: CC[C@@]1(O)C(=O)OCc2c1cc1n(c2=O)Cc2cc3c(CN4CCN(C(=O)CCCCCC5CCN(c6ccc(C(=O)N[C@H]7CC[C@H](Oc8ccc(C#N)c(Cl)c8C)CC7)nn6)CC5)CC4)c(O)ccc3nc2-1] |
| 51 | 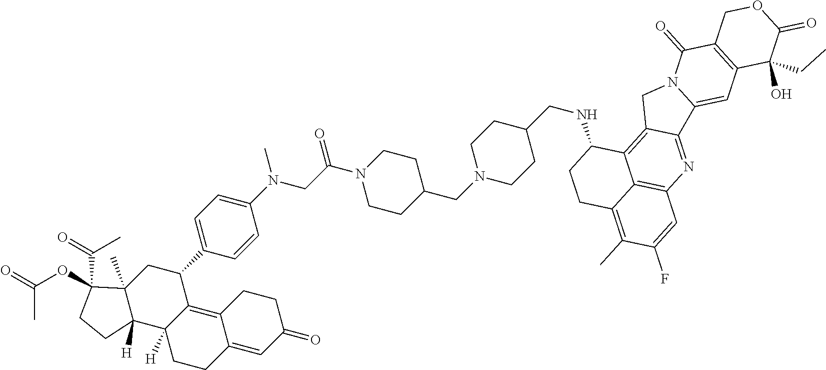 [SMILES: CC[C@@]1(O)C(=O)OCc2c1cc1n(c2=O)Cc2c-1nc1cc(F)c(C)c3c1c2[C@@H](NCC1CCN(CC2CCN(C(=O)CN(C)c4ccc([C@H]5C[C@@]6(C)[C@@H](CC[C@]6(OC(C)=O)C(C)=O)[C@@H]6CCC7=CC(=O)CCC7=C65)cc4)CC2)CC1)CC3] |
| 52 | 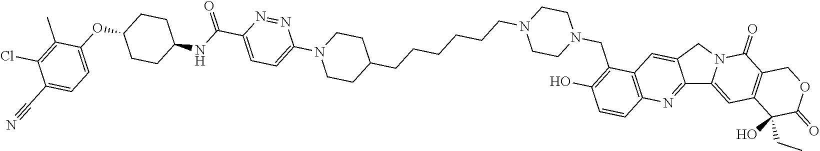 [SMILES: CC[C@@]1(O)C(=O)OCc2c1cc1n(c2=O)Cc2cc3c(CN4CCN(CCCCCCC5CCN(c6ccc(C(=O)N[C@H]7CC[C@H](Oc8ccc(C#N)c(Cl)c8C)CC7)nn6)CC5)CC4)c(O)ccc3nc2-1] |
| 53 | 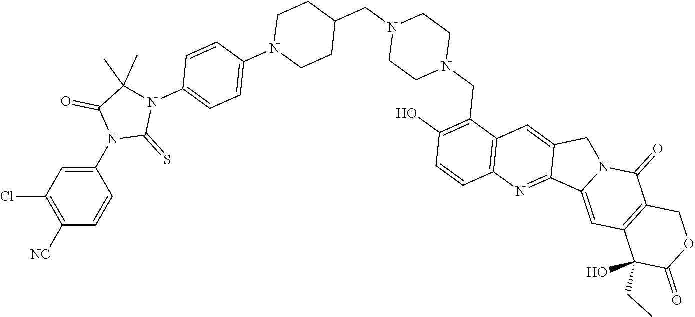 [SMILES: CC[C@@]1(O)C(=O)OCc2c1cc1n(c2=O)Cc2cc3c(CN4CCN(CC5CCN(c6ccc(N7C(=S)N(c8ccc(C#N)c(Cl)c8)C(=O)C7(C)C)cc6)CC5)CC4)c(O)ccc3nc2-1] |
| 54 | 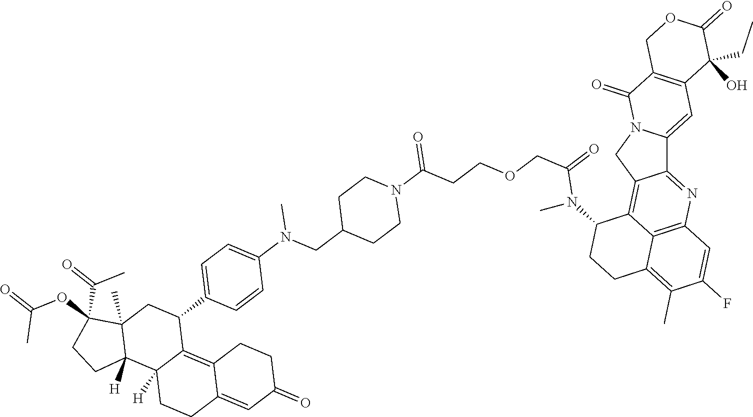 [SMILES: CC[C@@]1(O)C(=O)OCc2c1cc1n(c2=O)Cc2c-1nc1cc(F)c(C)c3c1c2[C@@H](N(C)C(=O)COCCC(=O)N1CCC(CN(C)c2ccc([C@H]4C[C@@]5(C)[C@@H](CC[C@]5(OC(C)=O)C(C)=O)[C@@H]5CCC6=CC(=O)CCC6=C54)cc2)CC1)CC3] |
| 55 | 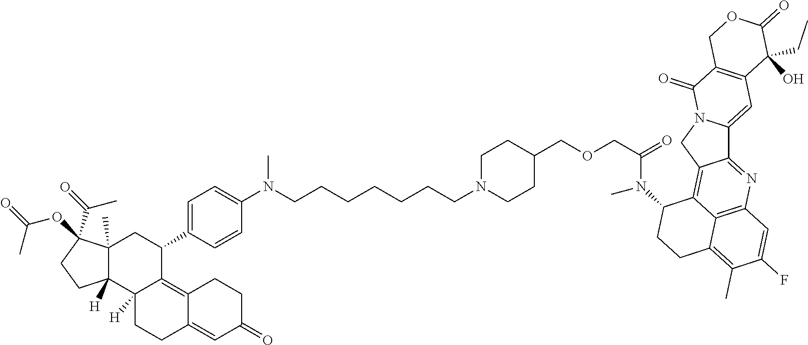 [SMILES: CC[C@@]1(O)C(=O)OCc2c1cc1n(c2=O)Cc2c-1nc1cc(F)c(C)c3c1c2[C@@H](N(C)C(=O)COCC1CCN(CCCCCCCN(C)c2ccc([C@H]4C[C@@]5(C)[C@@H](CC[C@]5(OC(C)=O)C(C)=O)[C@@H]5CCC6=CC(=O)CCC6=C54)cc2)CC1)CC3] |
| 56 | 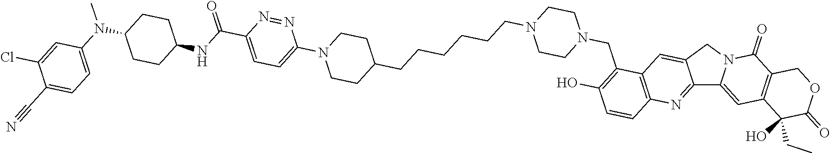 [SMILES: CC[C@@]1(O)C(=O)OCc2c1cc1n(c2=O)Cc2cc3c(CN4CCN(CCCCCCC5CCN(c6ccc(C(=O)N[C@H]7CC[C@H](N(C)c8ccc(C#N)c(Cl)c8)CC7)nn6)CC5)CC4)c(O)ccc3nc2-1] |
| 57 | 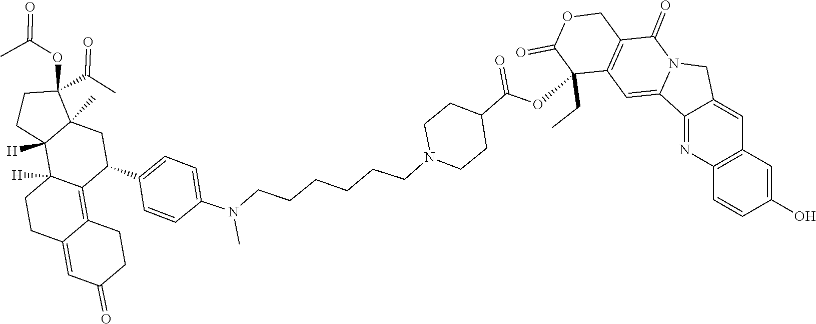 [SMILES: CC[C@@]1(OC(=O)C2CCN(CCCCCCN(C)c3ccc([C@H]4C[C@@]5(C)[C@@H](CC[C@]5(OC(C)=O)C(C)=O)[C@@H]5CCC6=CC(=O)CCC6=C54)cc3)CC2)C(=O)OCc2c1cc1n(c2=O)Cc2cc3cc(O)ccc3nc2-1] |
| 58 | 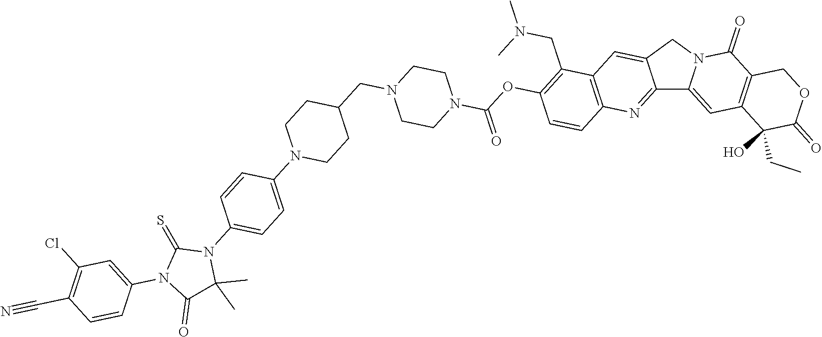 [SMILES: CC[C@@]1(O)C(=O)OCc2c1cc1n(c2=O)Cc2cc3c(CN(C)C)c(OC(=O)N4CCN(CC5CCN(c6ccc(N7C(=S)N(c8ccc(C#N)c(Cl)c8)C(=O)C7(C)C)cc6)CC5)CC4)ccc3nc2-1] |
| 59 | 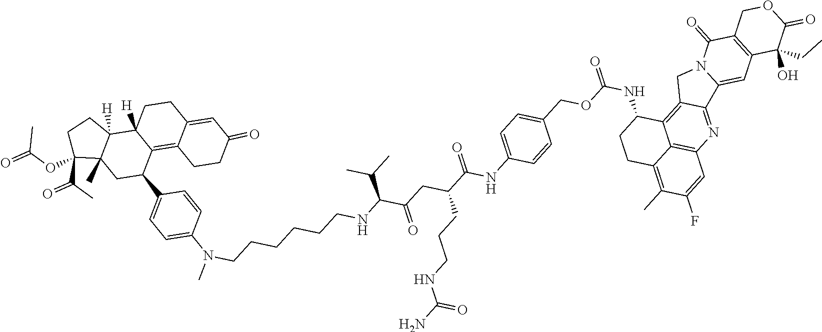 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)CC(=O)[C@@H](NCCCCCCN(C)c2ccc([C@H]4C[C@@]5(C)[C@@H](CC[C@]5(OC(C)=O)C(C)=O)[C@@H]5CCC6=CC(=O)CCC6=C54)cc2)C(C)C)cc1)CC3] |
| 60 | 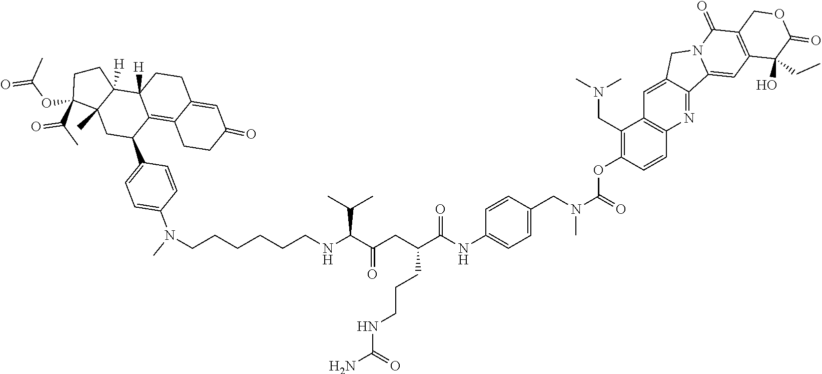 [SMILES: CC[C@@]1(O)C(=O)OCc2c1cc1n(c2=O)Cc2cc3c(CN(C)C)c(OC(=O)N(C)Cc4ccc(NC(=O)[C@H](CCCNC(N)=O)CC(=O)[C@@H](NCCCCCCN(C)c5ccc([C@H]6C[C@@]7(C)[C@@H](CC[C@]7(OC(C)=O)C(C)=O)[C@@H]7CCC8=CC(=O)CCC8=C76)cc5)C(C)C)cc4)ccc3nc2-1] |
| 61 | 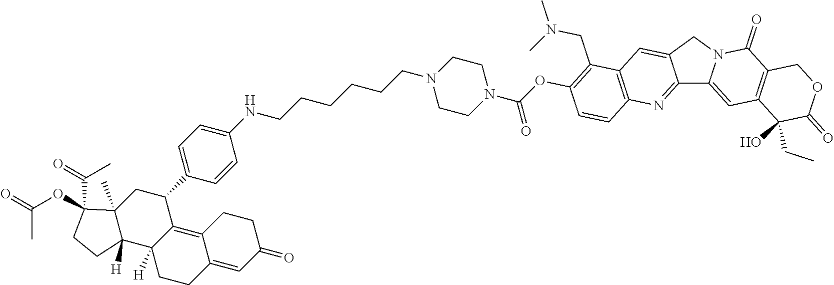 [SMILES: CC[C@@]1(O)C(=O)OCc2c1cc1n(c2=O)Cc2cc3c(CN(C)C)c(OC(=O)N4CCN(CCCCCCNc5ccc([C@H]6C[C@@]7(C)[C@@H](CC[C@]7(OC(C)=O)C(C)=O)[C@@H]7CCC8=CC(=O)CCC8=C76)cc5)CC4)ccc3nc2-1] |

### Methods of Treatment

Provided herein are compounds which can be used to treat, prevent, and/or delay the onset and/or development of cancer. Accordingly, in certain embodiments, provided is a method for the treatment of cancer, comprising administering to a subject in need of treatment a therapeutically-effective amount of a compound or composition described herein. Certain embodiments provide a method of potentiation of cytotoxic cancer therapy in a subject in recognized need of such treatment comprising administering to the subject a therapeutically acceptable amount of a compound or composition described herein.

It is contemplated that a patient having any cancer may benefit from being treated with the compounds and compositions described herein. Accordingly, in certain embodiments, the cancer is liver cancer, melanoma, Hodgkin's disease, non-Hodgkin's lymphomas, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, Waldenström macroglobulinemia, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, malignant melanoma, choriocarcinoma, mycosis fungoides, head neck carcinoma, osteogenic sarcoma, pancreatic carcinoma, acute granulocytic leukemia, hairy cell leukemia, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, trophoblastic neoplasms, or prostatic carcinoma. In certain embodiments, the cancer is bladder cancer, a blood cancer, such as leukemia (e.g., chronic leukemia, chronic lymphocytic leukemia (CLL, etc.) or lymphoma (e.g., Hodgkin lymphoma, non-Hodgkin lymphoma, low grade lymphoma, high grade lymphoma), lung cancer (e.g., small cell lung cancer), breast cancer, fallopian tube cancer, glioblastoma multiforme, head and neck cancer, esophageal cancer, ovarian cancer, pancreatic cancer, peritoneal cancer, prostate cancer, testicular cancer, skin cancer (e.g., melanoma) or uterine cancer. In certain embodiments, the cancer is bladder cancer, breast cancer, fallopian tube cancer, ovarian cancer, prostate cancer, peritoneal cancer, testicular cancer, endometrial cancer, or uterine cancer.

In certain embodiments, the cancer is chronic lymphocytic leukemia (CLL), Hodgkin lymphoma, non-Hodgkin lymphoma, Waldenström macroglobulinemia, polycythemia vera, trophoblastic neoplasms, and ovarian carcinoma.

In certain embodiments, the compounds and compositions as described herein are tailored to target cancers which overexpress a specific receptor, such as, but not limited to, androgen receptors, estrogen receptors, progesterone receptors, and/or glucocorticoid receptors by including an epitope which targets that specific nuclear receptor. The epitope can be derived from a steroid hormone or any non-steroidal drug which targets that particular receptor.

### Compositions

Compositions, including pharmaceutical compositions, of any of the compounds detailed herein are embraced by this disclosure. Thus, provided herein are pharmaceutical compositions comprising a compound of the disclosure, or a pharmaceutically acceptable salt thereof, and a pharmaceutically acceptable carrier or excipient. The pharmaceutical compositions provided herein may take a form suitable for oral, buccal, parenteral (e.g., intravenous, intramuscular, infusion or subcutaneous injection), nasal, topical or rectal administration, or a form suitable for administration by inhalation.

### Kits

Kits for use to achieve anti-cancer effects comprising a compound or composition described herein are provided. In certain embodiments, the kit comprises a unit dose of a compound or composition described herein and instructions for administering the same. In certain aspects, the kit further comprises a second drug suitable for anti-cancer therapy, or instructions for co-administering an additional anti-cancer therapy (such as radiation or gene therapy). In another aspect, kits for use to achieve anti-cancer effects comprise a low dose (e.g., less than about 500 mg/day, or less than about 400 mg/day, or less than about 300 mg/day, or less than about 200 mg/day) of a compound or composition described herein and a second drug suitable for anti-cancer therapy. In yet another variation, kits for use to achieve anti-cancer effects comprise a high dose (e.g., greater than about 500 mg/day) of a compound or composition as described herein and a second drug suitable for anti-cancer therapy.

### Methods of Manufacturing a Medicament

In a further aspect of the disclosure, use of the compounds and compositions described herein in the manufacture of a medicament is provided. In particular, the manufacture of a medicament for use in the treatment of cancer, or diseases or conditions which can be mediated, at least in part, by blocking DNA repair and/or transcription activation, such as by inhibition of one or more topoisomerase, are provided. Further, pharmaceutical compositions of a compound described herein are also intended for use in the manufacture of a medicament for use in treatment of diseases or conditions which can be mediated, at least in part, by inhibition of one or more topoisomerase.

### EXAMPLES

The disclosure is further illustrated by the following examples. The examples below are non-limiting are merely representative of various aspects of the disclosure. Solid and dotted wedges within the structures herein disclosed illustrate relative stereochemistry, with absolute stereochemistry depicted only when specifically stated or delineated.

Compounds having the structure of any compound, Formula, or any sub-formula described herein can be synthesized using standard synthetic techniques known to those of skill in the art. Compounds of the present disclosure can be synthesized using the general synthetic procedures set forth in the General Methods or the Synthetic Examples.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

### Example S1. Preparation of (S)-10-((4-(1-(6-(((1r,4r)-4-(3-Chloro-4-cyanophenoxy)cyclohexyl)carbamoyl)pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl Diisopropylcarbamate (Compound No. 1)

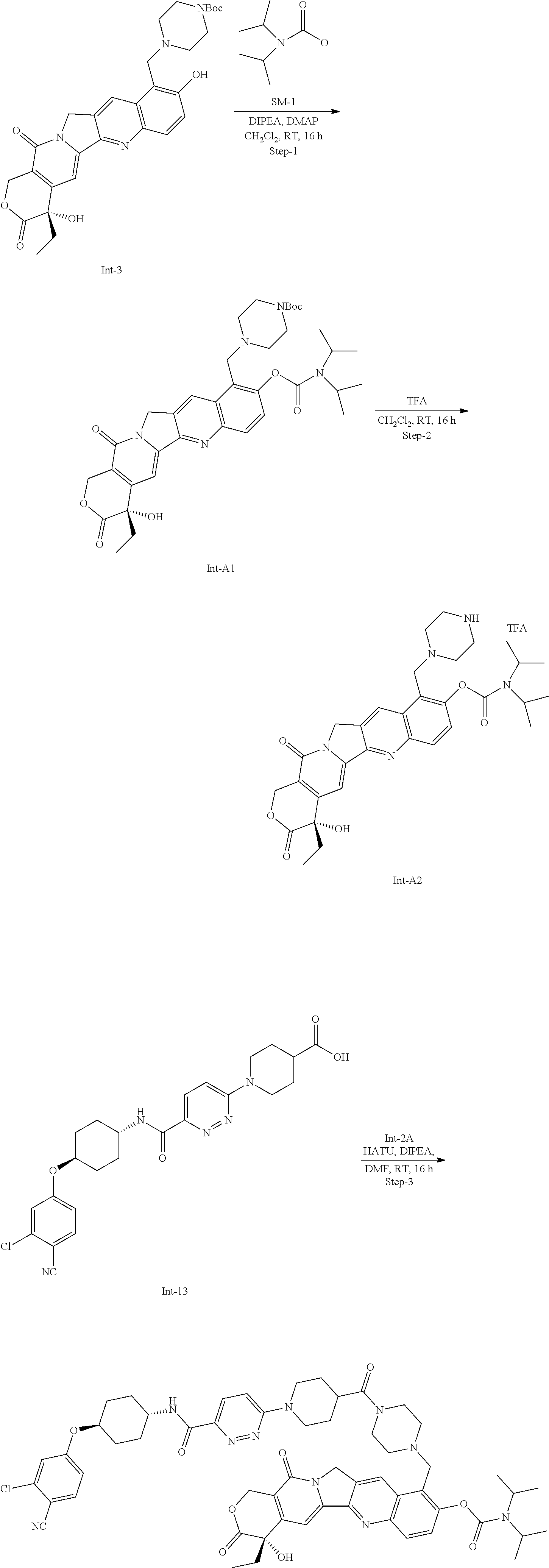

### Step-1: Preparation of tert-Butyl (S)-4-((9-((Diisopropylcarbamoyl)oxy)-4-ethyl-4-hydroxy-3,14-dioxo-3,4,12,14-tetrahydro-1H-pyrano[3′,4′:6,7]indolizino[1,2-b]quinolin-10-yl)methyl)piperazine-1-carboxylate (Int-A1)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-3, 300 mg, 0.53 mmol, 1.0 eq.) in DCM (5 mL) were added DIPEA (0.5 mL, 3.8 mmol, 5 eq.) and DMAP (20 mg, 0.16 mmol, 0.25 eq.) followed by addition of diisopropylcarbamic chloride (SM-1, 132 mg, 0.80 mmol, 1.5 eq.) in DCM (2 mL) at room temperature and stirred for 16 h. Progress of the reaction was monitored by TLC. After completion of the reaction, water (100 mL) was added and the aqueous reaction mixture was extracted with DCM (2×200 mL). The combined organic extract was washed with brine (100 mL), dried over anhydrous sodium sulfate, filtered and concentrated under vacuum to obtain the crude product. The crude obtained was purified by combiflash column eluting with 4% methanol in DCM to afford the product as pale yellow solid (Int-A1, 240 mg, 65%).

¹H NMR (400 MHz, DMSO-d₆) δ 9.02 (s, 1H), 8.14-8.16 (m, 1H), 7.60-7.63 (m, 1H), 7.38 (s. 1H), 6.54 (s, 1H), 5.46 (s, 2H), 5.32 (s, 2H), 3.88 (s, 2H), 3.26-3.42 (m, 7H), 2.40-2.45 (m, 3H), 1.84-1.92 (m, 2H), 1.39 (s, 9H), 1.33 (d, J=7.5 Hz, 12H), 1.09 (t, J=7.09 Hz, 3H). LCMS: 690.7 [M+H]⁺.

### Step-2: Preparation of (S)-4-Ethyl-4-hydroxy-3,14-dioxo-10-(piperazin-1-ylmethyl)-3,4,12,14-tetrahydro-1H-pyrano[3′,4′:6,7]indolizino[1,2-b]quinolin-9-yl Diisopropylcarbamate Trifluoroacetate Salt (Int-A2)

To a stirred solution of tert-butyl (S)-4-((9-((diisopropylcarbamoyl)oxy)-4-ethyl-4-hydroxy-3,14-dioxo-3,4,12,14-tetrahydro-1H-pyrano[3′,4′:6,7]indolizino[1,2-b]quinolin-10-yl)methyl)piperazine-1-carboxylate (Int-A1, 240 mg, 0.35 mmol, 1.0 eq.) in DCM (5 mL) under nitrogen atmosphere was added TFA (0.2 mL, 1.05 mmol, 3 eq.) at 0° C. The reaction mixture was allowed to stir and warm up to RT for 16 h. Progress of the reaction was monitored by TLC. After completion of the reaction, solvents were evaporated under reduced pressure, washed with diethyl ether (20 mL) and dried under vacuum to afford Int-A2 (220 mg, 94%) as an off-white solid.

¹H NMR (400 MHz, DMSO-d₆) δ 9.02 (s, 1H), 8.52 (br s, 2H), 8.13 (d, J=9.29 Hz, 1H), 7.58 (d, J=9.29 Hz, 1H), 7.34 (s, 1H), 6.50 (br s, 1H), 5.44 (s, 2H), 5.31 (s, 2H), 4.23 (br s, 2H), 3.90-3.96 (m, 4H), 3.05-3.15 (m, 4H), 2.62-2.67 (m, 2H), 1.81-1.95 (m, 2H), 1.37 (d, J=6.85 Hz, 6H), 1.29 (d, J=6.36 Hz, 6H), 0.84-0.97 (d, J=8.5 Hz, 3H). LCMS: 590.2 [M+H]⁺.

### Step-3: Preparation of (S)-10-((4-(1-(6-(((1r,4r)-4-(3-Chloro-4-cyanophenoxy)cyclohexyl)carbamoyl)pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl Diisopropylcarbamate

To a stirred solution of Int-A2 (200 mg, 0.33 mmol, 1.0 eq.) and Int-13 (CAS Registry No. 2740523-67-3; 164 mg, 0.33 mmol, 1.0 eq.) in DMF (5 mL) were added HATU (194 mg, 0.51 mmol, 1.5 eq.) and DIPEA (0.09 mL, 0.66 mmol, 2 eq.) at room temperature and the resulting reaction mixture was allowed to stir for 16 h. Progress of the reaction was monitored by TLC. After completion of the reaction, water (100 mL) was added and the aqueous mixture was extracted with ethyl acetate (2×50 mL). The combined organic extract was washed with water (100 mL), brine (100 mL) and dried over anhydrous sodium sulfate, filtered and concentrated under vacuum to obtain the crude product. The crude material was purified by combiflash column chromatography eluting with 3% methanol in DCM to afford the title compound (125 mg, 48%) as a pale yellow solid.

¹H NMR (400 MHz, DMSO-d₆) δ 9.10 (br s, 1H), 8.57 (d, J=8.25 Hz, 1H), 8.16-8.36 (m, 1H), 7.79-7.88 (m, 2H), 7.68 (br dd, J=6.63, 4.50 Hz, 1H), 7.35-7.40 (m, 3H), 7.13 (dd, J=8.82, 2.44 Hz, 1H), 6.39-6.68 (m, 1H), 5.44 (s, 2H), 5.35 (s, 2H), 4.46-4.56 (m, 3H), 4.26-4.35 (m, 4H), 3.78-3.96 (m, 4H), 3.33-3.63 (m, 3H), 2.92-3.20 (m, 4H), 2.04-2.16 (m, 2H), 1.84-1.97 (m, 4H), 1.46-1.77 (m, 9H), 1.33 (br dd, J=12.57, 6.32 Hz, 12H), 0.90 (t, J=7.32 Hz, 3H). LCMS: 1055.4 [M+H]⁺. HPLC purity 98.9%.

### Example S2. Preparation of (S)-10-((4-(1-(6-(((1r,4r)-4-(3-Chloro-4-cyanophenoxy)cyclohexyl)carbamoyl)pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl Methyl(phenyl)carbamate (Compound No. 2)

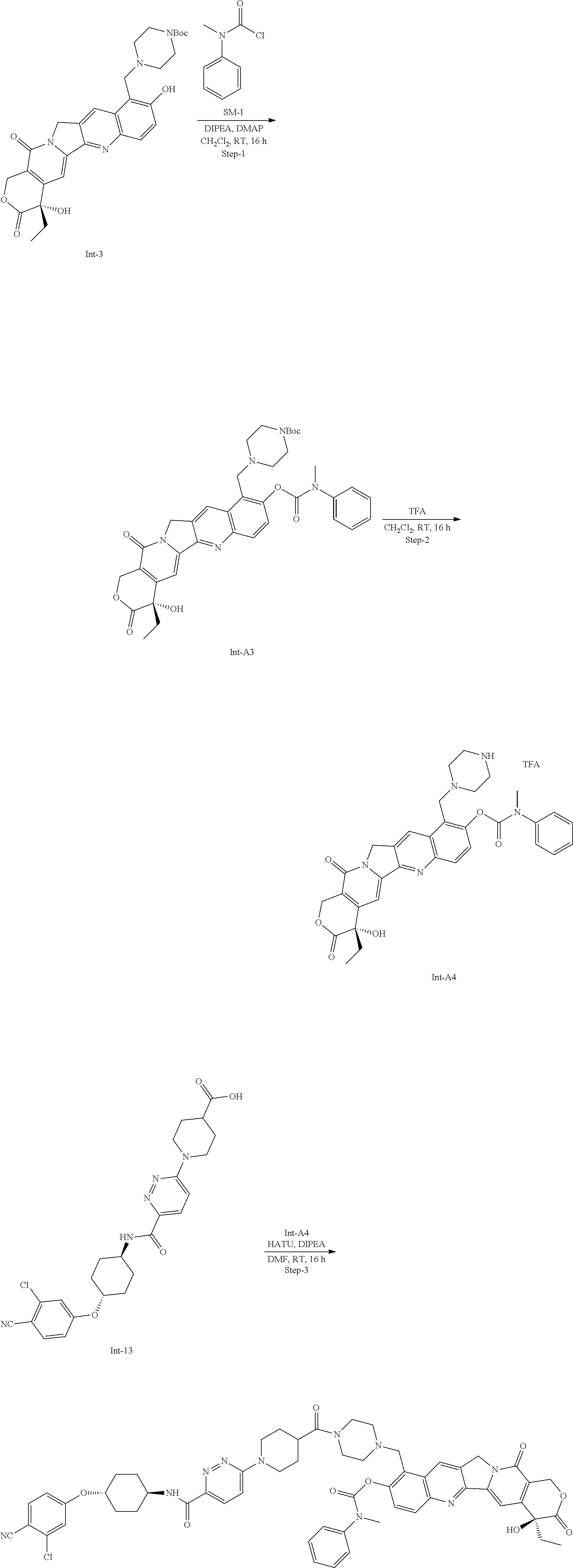

### Step-1: Preparation of tert-Butyl (S)-4-((4-Ethyl-4-hydroxy-9-((methyl(phenyl)carbamoyl)oxy)-3,14-dioxo-3,4,12,14-tetrahydro-1H-pyrano[3′,4′:6,7]indolizino[1,2-b]quinolin-10-yl)methyl)piperazine-1-carboxylate (Int-A3)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-3, 300 mg, 0.53 mmol, 1.0 eq.) in DCM (15 mL) were added DIPEA (0.3 mL, 1.60 mmol, 3 eq.) and DMAP (20 mg, 0.16 mmol, 0.25 eq.) followed by addition of methyl(phenyl)carbamic chloride (SM-1, 135 mg, 0.80 mmol, 1.5 eq.) in DCM (5 mL) at room temperature. The resulting reaction mixture was then allowed to stir for 16 h. Progress of the reaction was monitored by TLC. After completion of the reaction, the reaction mixture was diluted in water (100 mL) and extracted with DCM (2×200 mL). The combined organic extract was washed with brine (100 mL) and dried over anhydrous sodium sulfate, filtered and concentrated under vacuum to obtain the crude product. The crude obtained was purified by combiflash column using 6% methanol in DCM to afford Int-A3 (260 mg, 70%) as an off-white solid.

¹H NMR (400 MHz, DMSO-d₆) δ 8.96 (br s, 1H), 8.06-8.19 (m, 2H), 7.74 (d, J=9.13 Hz, 1H), 7.55 (br d, J=7.63 Hz, 1H), 7.44-7.46 (m, 1H), 7.28-7.35 (m, 2H), 6.57-6.62 (m, 1H), 6.48-6.54 (m, 1H), 5.42 (s, 2H), 5.29 (s, 2H), 3.69-3.82 (m, 1H), 3.38-3.40 (m, 3H), 3.16-3.19 (m, 3H), 2.95 (s, 3H), 2.65 (d, J=5.13 Hz, 1H), 2.12-2.27 (m, 3H), 1.85-1.90 (m, 1H), 1.41 (s, 9H), 0.88 (t, J=7.32 Hz, 3H). LCMS: 696.2 [M+H]⁺.

### Step-2: Preparation of (S)-4-Ethyl-4-hydroxy-3,14-dioxo-10-(piperazin-1-ylmethyl)-3,4,12,14-tetrahydro-1H-pyrano[3′,4′:6,7]indolizino[1,2-b]quinolin-9-yl Methyl(phenyl)carbamate Trifluoroacetate Salt (Int-A4)

To a stirred solution of tert-butyl (S)-4-((4-ethyl-4-hydroxy-9-((methyl(phenyl)carbamoyl)oxy)-3,14-dioxo-3,4,12,14-tetrahydro-1H-pyrano[3′,4′:6,7]indolizino [1,2-b]quinolin-10-yl)methyl)piperazine-1-carboxylate (Int-A3, 250 mg, 0.35 mmol, 1.0 eq.) in DCM (10 mL) under nitrogen atmosphere was added TFA (0.27 mL, 3.5 mmol, 10 eq.) at 0° C. The reaction mixture was allowed to warm up to room temperature and stir for 16 h. Progress of the reaction was monitored by TLC. After completion of the reaction, solvents were evaporated under reduced pressure, washed with diethyl ether (20 mL) and dried under vacuum to afford Int-A4 (210 mg, 98%) as an off-white solid.

¹H NMR (400 MHz, DMSO-d₆) δ 8.99 (br s, 1H), 8.56 (br s, 2H), 8.15 (br d, J=9.29 Hz, 1H), 7.75 (br d, J=8.80 Hz, 1H), 7.53-7.58 (m, 3H), 7.49 (br t, J=7.58 Hz, 1H), 7.34 (s, 1H), 6.50 (br s, 1H), 5.43 (s, 2H), 5.29 (br s, 2H), 3.81-3.96 (m, 3H), 3.42 (br s, 3H), 2.98-3.01 (m, 5H), 2.52-2.56 (m, 3H), 1.80-1.96 (m, 2H), 0.89 (br t, J=7.09 Hz, 3H).

### Step-3: Preparation of (S)-10-((4-(1-(6-(((1r,4r)-4-(3-Chloro-4-cyanophenoxy)cyclohexyl)carbamoyl)pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl Methyl(phenyl)carbamate

To a stirred solution of (S)-4-ethyl-4-hydroxy-3,14-dioxo-10-(piperazin-1-ylmethyl)-3,4,12,14-tetrahydro-1H-pyrano[3′,4′:6,7]indolizino[1,2-b]quinolin-9-yl methyl(phenyl)carbamate trifluoroacetate (Int-A4, 200 mg, 0.33 mmol, 1.0 eq.) and 1-(6-(((1r,4r)-4-(3-chloro-4-cyanophenoxy)cyclohexyl)carbamoyl)pyridazin-3-yl)piperidine-4-carboxylic acid (Int-13, 200 mg, 0.40 mmol, 1.2 eq.) in DMF (5 mL) were added HATU (240 mg, 0.67 mmol, 1.5 eq.) and DIPEA (0.17 mL, 1 mmol, 3 eq.) at room temperature. The resulting reaction mixture was allowed to stir for 16 h. Progress of the reaction was monitored by TLC. After completion of the reaction, the reaction mixture was washed with water (20 mL) and extracted with 10% methanol in DCM (2×50 mL). The combined organic extract was washed with brine (100 mL), dried over anhydrous sodium sulfate, filtered and concentrated under vacuum to obtain the crude product. The crude obtained was purified by prep. HPLC in ammonium bicarbonate in water/acetonitrile mobile phase to afford the title compound (75 mg, 21%) as an off-white solid.

¹H NMR (400 MHz, DMSO-d₆) δ 8.99 (br s, 1H), 8.59 (d, J=8.25 Hz, 1H), 8.14 (d, J=9.13 Hz, 1H), 7.84 (dd, J=10.94, 9.19 Hz, 2H), 7.76 (d, J=9.26 Hz, 1H), 7.55 (br d, J=7.75 Hz, 2H), 7.47 (t, J=7.75 Hz, 2H), 7.31-7.41 (m, 4H), 7.13 (dd, J=8.76, 2.38 Hz, 1H), 6.51 (s, 1H), 5.43 (s, 2H), 5.30 (s, 2H), 4.44-4.57 (m, 3H), 3.72-3.94 (m, 3H), 3.42 (s, 3H), 3.38-3.41 (m, 4H), 3.07-3.18 (m, 2H), 2.91-3.01 (m, 1H), 2.07-2.29 (m, 5H), 1.80-1.97 (m, 4H), 1.43-1.76 (m, 9H), 0.89 (t, J=7.32 Hz, 3H). LCMS: 1061.4 [M+H]⁺. HPLC purity 98.2%.

### Example S3. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 3-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-3,6-diazabicyclo[3.1.1]heptane-6-carboxylate (Compound No. 3)

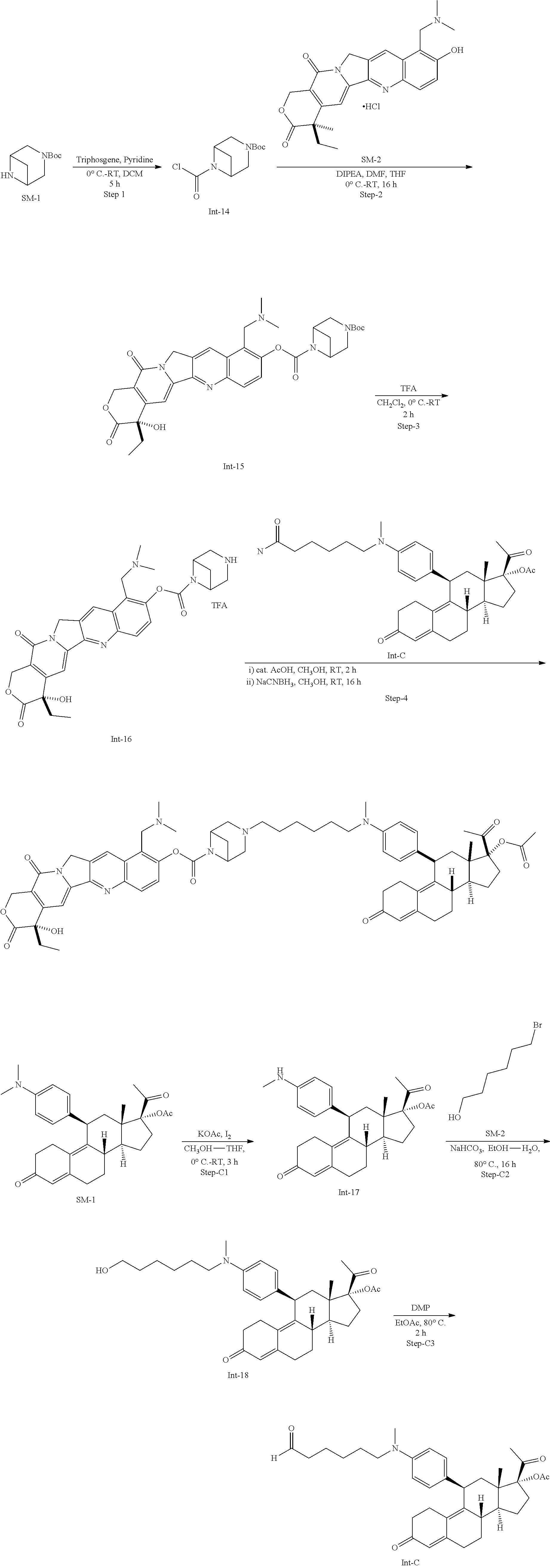

### Step-1: Preparation of tert-Butyl 6-(chlorocarbonyl)-3,6-diazabicyclo[3.1.1]heptane-3-carboxylate (Int-14)

To a stirred solution of tert-butyl 3,8-diazabicyclo[3.2.1]octane-3-carboxylate (SM-1, 1.0 g, 5.05 mmol, 1.0 eq.) in DCM (10 mL) were added pyridine (0.81 mL, 10.10 mmol, 2 eq.) and triphosgene (0.45 g, 1.51 mmol, 0.3 eq.) solution in DCM (5 mL) dropwise over a period of 10 min at 0° C. The reaction mixture was allowed to warm up to room temperature and stir for 2 h. Progress of the reaction was monitored by TLC (non-polar spot was observed). After completion of the reaction, the reaction mixture was poured into ice cold water (20 mL) and extracted with DCM (2×15 mL). The combined organic extract was washed with brine (50 mL) and dried over anhydrous sodium sulfate, filtered and concentrated under vacuum to afford Int-14 (1.0 g, crude) as light yellow solid which was used in next step without further purification.

¹H NMR (400 MHz, DMSO-d₆) δ 4.63-4.76 (m, 2H), 3.57-3.84 (m, 4H), 2.91-2.99 (m, 1H), 1.70-1.78 (m, 1H), 1.44 (s, 9H).

### Step-2: Preparation of 3-(tert-Butyl) 6-((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) 3,6-Diazabicyclo[3.1.1]heptane-3,6-dicarboxylate (Int-15)

To a stirred solution of tert-butyl 6-(chlorocarbonyl)-3,6-diazabicyclo[3.1.1]heptane-3-carboxylate (Int-16, 0.92 g, 3.56 mmol, 1.5 eq.) and (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione HCl salt (SM-2, 1 g, 2.37 mmol, 1 eq.) in THF (10 mL) and DMF (10 mL) was added DIPEA (2.12 mL, 11.87 mmol, 5 eq.) at 0° C. The reaction mixture was allowed to warm up to room temperature and stir for 16 h. Progress of the reaction was monitored by TLC. After completion of the reaction, the reaction mixture was quenched with water (20 mL), extracted with ethyl acetate (2×20 mL). The combined organic layer was dried over Na₂SO₄, concentrated under reduced pressure to get the crude product. The crude obtained was purified by combiflash column chromatography eluting with 6% methanol in DCM to afford Int-15 (650 mg, 42%) as a light-yellow solid.

¹H NMR (400 MHz, DMSO-d₆) δ 8.96 (s, 1H), 8.10 (d, J=8.63 Hz, 1H), 7.51 (d, J=9.01 Hz, 1H), 7.34 (s, 1H), 6.51 (s, 1H), 5.43 (s, 2H), 5.31 (s, 2H), 3.70-3.87 (m, 2H), 3.55-3.66 (m, 6H), 2.69-2.77 (m, 1H), 2.12-2.24 (m, 6H), 1.81-1.93 (m, 2H), 1.54-1.59 (m, 1H), 1.47 (s, 9H), 0.89 (t, J=7.38 Hz, 3H). LCMS: 646.50 [M+H]⁺.

### Step-3: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 3,6-Diazabicyclo[3.1.1]heptane-6-carboxylate Trifluoroacetate Salt (Int-16)

To a stirred solution of 3-(tert-butyl) 6-((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) 3,6-diazabicyclo[3.1.1]heptane-3,6-dicarboxylate (Int-17, 650 mg, 1.0 mmol, 1.0 eq.) in DCM (10 mL) was added TFA (0.57 mL, 7.55 mmol, 7.5 eq.) at 0° C. The reaction mixture was allowed to warm up to room temperature and stir for 16 h. Progress of the reaction was monitored by TLC. After completion of the reaction, solvents were evaporated under reduced pressure to get the crude product, which triturated with ethyl acetate (30 mL), filtered and the collected solid was dried under vacuum to afford Int-16 (500 mg, 75%) as a yellow solid.

¹H NMR (400 MHz, D₂O) δ 8.99 (s, 1H), 8.33 (d, J=9.38 Hz, 1H), 7.95 (d, J=9.26 Hz, 1H), 7.63 (s, 1H), 5.29-5.60 (m, 4H), 4.92-5.00 (m, 2H), 3.97-4.24 (m, 3H), 3.72-3.88 (m, 3H), 3.19-3.31 (m, 2H), 3.05 (s, 3H), 3.02 (s, 4H), 1.93-2.09 (m, 3H), 0.98 (t, J=7.19 Hz, 3H). LCMS: 546.2 & 547.2 [M+H]⁺.

### Step-C1: Preparation of (8S,11R,13S,14S,17R)-17-Acetyl-13-methyl-11-(4-(methylamino)phenyl)-3-oxo-2,3,6,7,8,11,12,13,14,15,16,17-dodecahydro-1H-cyclopenta[a]phenanthren-17-yl Acetate (Int-17)

To a stirred solution of SM-1 (10 g, 21 mmol, 1.0 eq.) in methanol (150 mL) and THF (150 mL) were added KOAc (20.6 g, 210 mmol, 10 eq.) and Iodine (13.1 g, 105 mmol, 5 eq.) at 0° C. The reaction mixture was allowed to warm up to room temperature and stir for 3 h. Progress of the reaction was monitored by TLC. After completion of the reaction, the reaction mixture was quenched with sodium thiosulfate (Na₂S₂O₃) solution (50 g in 30 mL water) and extracted with ethyl acetate (2×200 mL). The combined organic extract was washed with brine (100 mL) and dried over anhydrous sodium sulfate, filtered and concentrated under vacuum to afford Int-17 (8.0 g, 82%) as an off-white solid which was used in next step without further purification.

¹H NMR (400 MHz, DMSO-d₆) δ 11.91 (br s, 1H), 6.91 (d, J=8.31 Hz, 2H), 6.44 (d, J=8.31 Hz, 2H), 5.67 (s, 1H), 4.37 (m, 1H), 2.75 (s, 2H), 2.61 (d, J=4.40 Hz, 3H), 2.30-2.40 (m, 1H), 2.07-2.16 (s, 5H), 1.99 (s, 6H), 1.63-1.77 (m, 2H), 1.21-1.45 (m, 5H), 0.86 (t, J=6.60 Hz, 1H), 0.16-0.28 (m, 3H). LCMS: 462.28 [M+H]⁺.

### Step-C2: Preparation of (8S,11R,13S,14S,17R)-17-Acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl Acetate (Int-18)

To a solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-17, 4 g, 8.67 mmol, 1.0 eq.) and 6-bromohexan-1-ol (SM-2, 7.81 g, 43.38 mmol, 5 eq.) in ethanol (40 mL) and water (40 mL) was added NaHCO₃ (7.37 g, 86.76 mmol, 10 eq.) at room temperature. The reaction mixture was heated to 80° C. and allowed to stir for 16 h. Progress of the reaction was monitored by TLC. After completion of the reaction, the reaction mixture was filtered through a pad of celite bed and washed with ethyl acetate (40 mL). The filtrate was concentrated under reduced pressure, diluted with water (120 mL) and extracted with ethyl acetate (2×200 mL). The combined organic extract was washed with brine (100 mL) and dried over anhydrous sodium sulfate, filtered and concentrated under vacuum to obtain the crude product. The crude obtained was purified by combiflash chromatography eluting with 70% ethyl acetate in heptane to afford Int-18 (2.6 g, 53%) as an off-white solid.

¹H NMR (400 MHz, DMSO-d₆) δ 6.98 (d, J=7.89 Hz, 2H), 6.58 (d, J=7.89 Hz, 2H), 5.67 (br s, 1H), 4.24-4.51 (m, 2H), 3.36 (d, J=5.70 Hz, 2H), 3.23 (d, J=6.58 Hz, 2H), 2.69-2.86 (m, 4H), 2.55 (s, 3H), 2.29-2.44 (m, 1H), 2.05-2.26 (m, 5H), 1.87-2.04 (m, 6H), 1.63-1.77 (m, 2H), 1.34-1.49 (m, 6H), 1.27 (br s, 6H), 0.23 (br s, 3H). LCMS: 562.40 [M+H]⁺.

### Step-C3: Preparation of (8S,11R,13S,14S,17R)-17-Acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl Acetate (Int-C)

To a stirred solution of Int-18 (500 mg, 0.891 mmol, 1 eq.) in ethyl acetate (40 mL), was added Dess-Martin periodinane (DMP) (1.1 g, 2.67 mmol, 3 eq.) portionwise at 0° C. The reaction mixture was heated to 80° C. for 2 h. Progress of the reaction was monitored by TLC. After completion of the reaction, the reaction mixture was quenched with 50% aqueous Na₂S₂O₃ solution (10 mL), sat. NaHCO₃ solution (15 mL) and extracted with ethyl acetate (2×25 mL). The combined organic extract was washed with brine (100 mL), dried over anhydrous sodium sulfate, filtered and concentrated under vacuum to afford Int-C (450 mg, 92%) as a brown solid.

¹H NMR (400 MHz, DMSO-d₆) δ 9.64 (s, 1H), 6.98 (d, J=8.31 Hz, 2H), 6.58 (d, J=8.80 Hz, 2H), 5.67 (s, 1H), 4.39 (d, J=5.87 Hz, 1H), 3.22 (t, J=6.60 Hz, 2H), 2.55-2.80 (m, 5H), 2.51-2.54 (m, 2H), 2.40 (t, J=7.09 Hz, 2H), 1.96-2.15 (m, 12H), 1.56-1.69 (m, 2H), 1.11-1.59 (m, 10H), 0.23 (s, 3H). LCMS: 560.4 [M+H]⁺.

### Step-4: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 3-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)-hexyl)-3,6-diazabicyclo[3.1.1]heptane-6-carboxylate

To a stirred solution of (8S,11R,13S,14S,17R)-17-acetyl-13-methyl-11-(4-(methyl(6-oxohexyl)amino)-phenyl)-3-oxo-2,3,6,7,8,11,12,13,14,15,16,17-dodecahydro-1H-cyclopenta[a]phenanthren-17-yl acetate (Int-C, 350 mg, 0.531 mmol, 1 eq.) and (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 3,6-diazabicyclo[3.1.1]heptane-6-carboxylate trifluoroacetate salt (Int-16, 296 mg, 0.531 mmol, 1 eq.) in methanol (7 mL) was added glacial acetic acid (0.1 mL) at room temperature and stirred for an additional 2 h. Sodium cyanoborohydride (65.8 mg, 1.06 mmol, 2 eq.) was then added at 0° C. and the reaction mixture was allowed to warm up to room temperature and stir for 16 h. Progress of the reaction was monitored by TLC. After completion of the reaction, solvents were evaporated under reduced pressure, quenched with ice cold water (20 mL) and extracted with 10% methanol in DCM (2×20 mL). The combined organic extract was washed with brine (100 mL), dried over anhydrous sodium sulfate, filtered and concentrated under vacuum to obtain the crude product. The crude obtained was purified by prep. HPLC purification method eluting with Mobile phase A: 0.1% FA in water and Mobile phase B: Acetonitrile to afford the title compound (20 mg, 3%) as an off-white solid.

¹H NMR (400 MHz, DMSO-d₆) δ 8.95 (s, 1H), 8.13 (d, J=9.26 Hz, 1H), 7.70 (d, J=9.13 Hz, 1H), 7.35 (s, 1H), 6.97 (d, J=8.63 Hz, 2H), 6.58 (d, J=8.63 Hz, 2H), 6.52 (s, 1H), 5.62-5.70 (m, 1H), 5.43 (s, 2H), 5.32 (s, 2H), 4.35-4.43 (m, 1H), 3.88-3.97 (m, 1H), 3.72-3.85 (m, 2H), 3.52-3.71 (m, 4H), 3.34-3.42 (m, 1H), 3.21-3.28 (m, 2H), 2.82 (s, 3H), 2.65-2.79 (m, 2H), 2.54-2.64 (m, 2H), 2.28-2.39 (m, 1H), 2.19 (s, 6H), 2.11-2.17 (m, 3H), 2.09 (s, 3H), 1.96-2.01 (m, 4H), 1.83-1.93 (m, 3H), 1.63-1.77 (m, 2H), 1.54-1.61 (m, 1H), 1.42-1.53 (m, 2H), 1.21-1.41 (m, 11H), 0.89 (t, J=7.32 Hz, 3H), 0.22 (s, 3H), -0.06 (s, 2H). LCMS: 1089.57 [M+H]⁺. HPLC purity 86.1%

### Example S4. Preparation of 2-(2-(((5S,8R,9S,10S,13S,14S,17S)-10,13-Dimethyl-3-oxohexadecahydro-1H-cyclopenta[a]phenanthren-17-yl)oxy)-N-methylacetamido)-N—(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)-N,2-dimethylpropanamide (Compound No. 4)

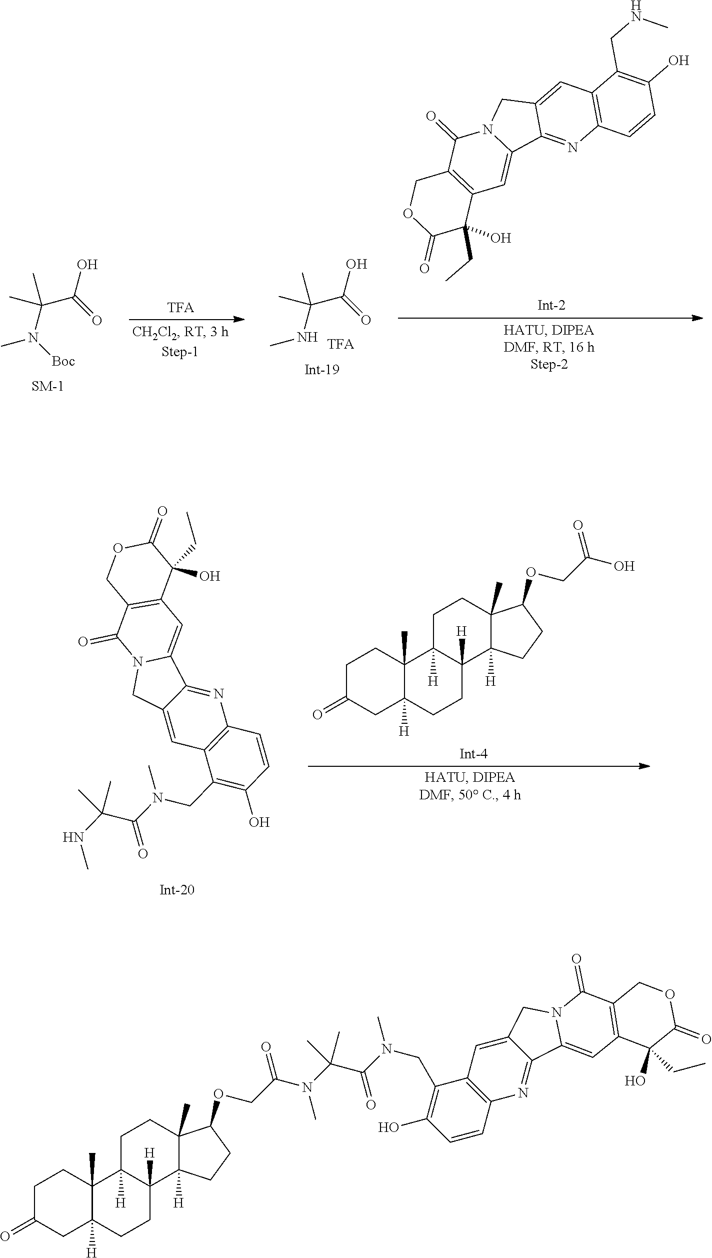

### Step-1: Preparation of 2-Methyl-2-(methylamino)propanoic acid Trifluoroacetate Salt (Int-19)

To a stirred solution of 2-((tert-butoxycarbonyl)(methyl)amino)-2-methylpropanoic acid (SM-1, 500 mg, 2.30 mmol, 1.0 eq.) in DCM (10 mL) under nitrogen atmosphere was added TFA (1.7 mL) at 0° C. The reaction mixture was allowed to warm up to room temperature and stir for 3 h. Progress of the reaction was monitored by TLC. After completion of the reaction, solvents were evaporated under reduced pressure, reaction mixture washed with diethyl ether (2×20 mL) and dried under vacuum to afford Int-19 (450 mg, 93%) as an off-white solid.

¹H NMR (400 MHz, DMSO-d₆) δ 14.125 (br s, 1H), 9.09 (br s, 2H), 2.51 (d, J=11.25 Hz, 3H), 1.40 (s, 6H). LCMS: 118.08 [M+H]⁺.

### Step-2: Preparation of (S)—N-((4-Ethyl-4,9-dihydroxy-3,14-dioxo-3,4,12,14-tetrahydro-1H-pyrano[3′,4′:6,7]-indolizino[1,2-b]quinolin-10-yl)methyl)-N,2-dimethyl-2-(methylamino)propanamide (Int-20)

To a stirred solution of (S)-4-ethyl-4,9-dihydroxy-10-((methylamino)methyl)-1,12-dihydro-14H-pyrano[3′,4′:6,7]indolizino[1,2-b]quinoline-3,14(4H)-dione (Int-2, 400 mg, 0.98 mmol, 1.0 eq.) and 2-methyl-2-(methylamino)propanoic acid trifluoroacetate salt (Int-19, 137 mg, 1.17 mmol, 1.2 eq.) in DMF (2 mL) were added HATU (703 mg, 1.96 mmol, 2.0 eq.) and DIPEA (0.5 mL, 2.94 mmol, 3 eq.) at room temperature and the resulting reaction mixture was allowed to stir for 16 h. Progress of the reaction was monitored by TLC. After completion of the reaction, the reaction mixture was washed with water (60 mL) and extracted with ethyl acetate (2×100 mL). The combined organic extract was washed with brine (100 mL), dried over anhydrous sodium sulfate, filtered and concentrated under vacuum to obtain the crude product. The crude obtained was purified by combiflash column eluting with 6% methanol in DCM to afford Int-20 (210 mg, 42%) as an off-white solid.

¹H NMR (400 MHz, DMSO-d₆) δ 8.43 (br s, 1H), 8.27 (br d, J=7.83 Hz, 2H), 8.04 (br d, J=8.80 Hz, 1H), 7.60 (br d, J=9.29 Hz, 1H), 7.18-7.36 (m, 1H), 6.50 (br s, 1H), 5.41 (br s, 2H), 5.23 (br s, 2H), 5.09 (br s, 2H), 3.33-3.40 (m, 2H), 3.00 (br s, 3H), 2.33 (br s, 3H), 1.40 (s, 6H), 0.87 (t, J=7.32 Hz, 3H). LCMS: 507.45 [M+H]⁺.

### Step-3: Preparation of 2-(2-(((5S,8R,9S,10S,13S,14S,17S)-10,13-Dimethyl-3-oxohexadecahydro-1H-cyclopenta[a]phenanthren-17-yl)oxy)-N-methylacetamido)-N—(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)-N,2-dimethylpropanamide

To a stirred solution of (S)—N-((4-ethyl-4,9-dihydroxy-3,14-dioxo-3,4,12,14-tetrahydro-1H-pyrano[3′,4′:6,7]indolizino[1,2-b]quinolin-10-yl)methyl)-N,2-dimethyl-2-(methylamino)propenamide (Int-20, 150 mg, 0.29 mmol, 1.0 eq.) and 2-(((5S,8R,9S,10S,13S,14S,17S)-10,13-dimethyl-3-oxohexadecahydro-1H-cyclopenta[a]phenanthren-17-yl)oxy)acetic acid Int-4 (113 mg, 0.32 mmol, 1.1 eq.) in DMF (2 mL) were added HATU (212 mg, 0.59 mmol, 2.0 eq.) and DIPEA (0.15 mL, 0.88 mmol, 3 eq.) at room temperature. The reaction mixture was heated to 50° C. and stirred for 4h. Progress of the reaction was monitored by TLC. After completion of the reaction, water (10 mL) was added and the aqueous reaction mixture was extracted with ethyl acetate (2×100 mL). The combined organic extract was washed with water (20 mL), brine (20 mL), dried over anhydrous sodium sulfate, filtered and concentrated under vacuum to obtain the crude product. The crude obtained was purified by prep. HPLC in ammonium bicarbonate in water/acetonitrile as the mobile phase to afford the title compound (7 mg, 3%) as an off-white solid.

¹H NMR (400 MHz, DMSO-d₆) δ 10.50 (d, J=4.38 Hz, 1H), 8.57 (s, 1H), 7.99 (d, J=9.13 Hz, 1H), 7.52 (d, J=9.13 Hz, 1H), 7.27 (s, 1H), 6.46 (s, 1H), 5.41 (br s, 2H), 5.30 (br s, 2H), 5.09 (br d, J=12.38 Hz, 1H), 4.90 (br d, J=12.38 Hz, 1H), 3.79-3.93 (m, 2H), 2.81 (s, 3H), 2.59 (s, 3H), 2.25 (br d, J=14.13 Hz, 1H), 2.12 (br d, J=15.26 Hz, 1H), 1.79-1.93 (m, 4H), 1.48-1.50 (m, 3H), 1.45 (s, 6H), 1.21-1.33 (m, 4H), 1.06-1.20 (m, 4H), 0.92-1.03 (m, 2H), 0.91 (s, 3H), 0.75-0.90 (m, 6H), 0.40-0.52 (m, 1H), 0.22-0.39 (m, 2H), 0.17 (s, 3H). LCMS: 837.35 [M+H]⁺. HPLC purity 98.8%.

### Example S5. Preparation of (S)-4-Ethyl-4-hydroxy-10-((4-methylpiperazin-1-yl)methyl)-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Compound No. 5)

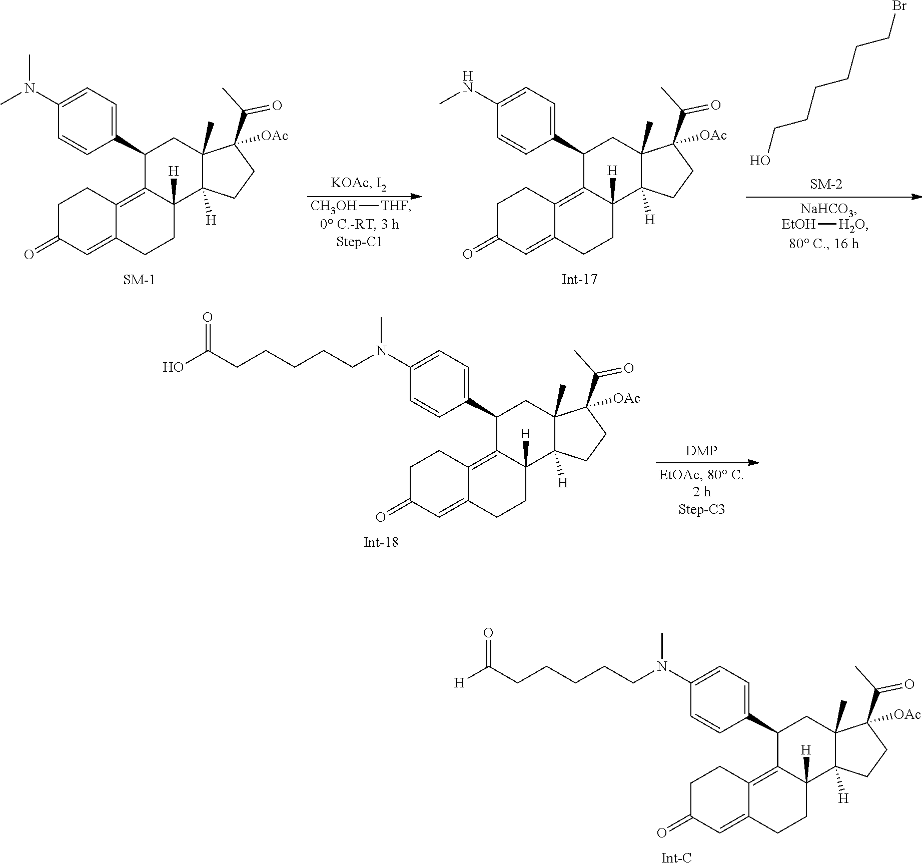

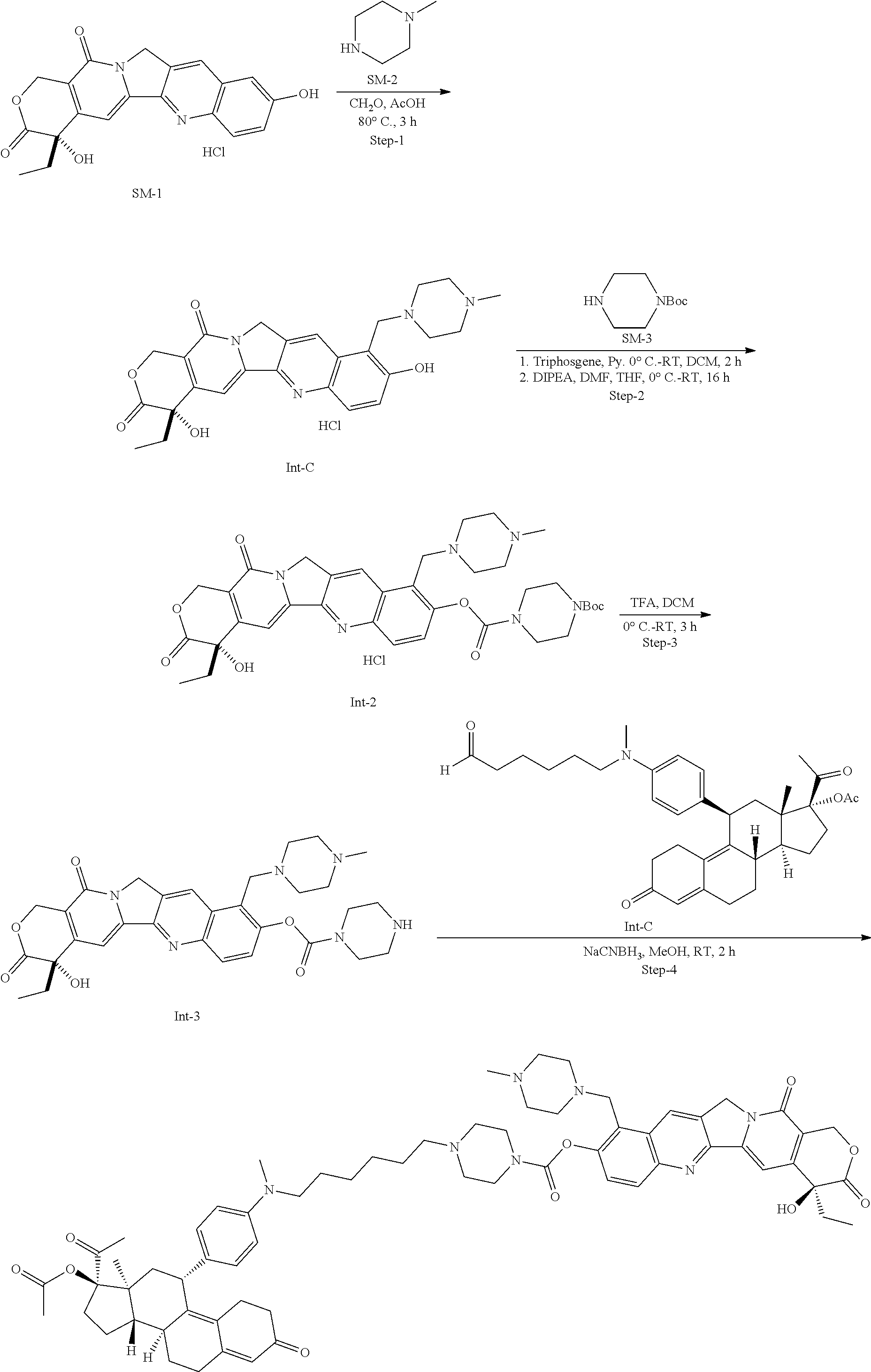

### Step-1: Preparation of (S)-4-Ethyl-4,9-dihydroxy-10-((4-methylpiperazin-1-yl)methyl)-1,12-dihydro-14H-pyrano[3′,4′:6,7]indolizino[1,2-b]quinoline-3,14(4H)-dione (Int-1)

To a stirred solution of (S)-4-ethyl-4,9-dihydroxy-1,12-dihydro-14H-pyrano[3′,4′:6,7]indolizino[1,2-b]quinoline-3,14(4H)-dione hydrochloride (SM-1, 10 g, 27.47 mmol, 1.0 eq.) in AcOH (100 mL, 10 vol), formaldehyde (3.95 mL, 41.20 mmol, 1.5 eq., 37% in H₂O) and 1-methylpiperazine (SM-2, 4.12 g, 41.20 mmol, 1.5 eq.) was added at ambient temperature under argon atmosphere. Resulting reaction mixture stirred at 80° C. until TLC indicated complete consumption of starting material. The reaction mixture was then diluted with ice cold water (250 mL), extracted with 10% MeOH in DCM (2×250 mL), combined organic layer washed with brine solution (200 mL), dried over anhydrous sodium sulfate, filtered and concentrated under reduced pressure. The product was washed with diethyl ether (100 mL) and dried to obtain (S)-4-ethyl-4,9-dihydroxy-10-((4-methylpiperazin-1-yl)methyl)-1,12-dihydro-14H-pyrano[3′,4′:6,7]indolizino[1,2-b]quinoline-3,14(4H)-dione (Int-1, 6 g, 46%) as an off-white solid.

¹H NMR (400 MHz, DMSO-d₆) δ 8.73 (s, 1H), 7.95-8.00 (m, 1H), 7.39-7.44 (m, 1H), 7.24-7.27 (m, 1H), 6.43-6.52 (m, 1H), 5.39-5.44 (m, 2H), 5.22-5.26 (m, 2H), 4.07-4.12 (m, 2H), 2.72-2.78 (m, 1H), 2.57-2.64 (m, 2H), 2.25-2.38 (m, 4H), 2.12-2.19 (m, 4H), 1.82-1.91 (m, 3H), 0.85-0.92 (m, 3H). LCMS: 477.3 [M+H]⁺.

### Step-2: Preparation of (S)-1-(tert-Butyl) 4-(4-Ethyl-4-hydroxy-10-((4-methylpiperazin-1-yl)methyl)-3,14-dioxo-3,4,12,14-tetrahydro-1H-pyrano[3′,4′:6,7]indolizino[1,2-b]quinolin-9-yl) Piperazine-1,4-dicarboxylate (Int-2)

To a stirred solution of tert-butyl piperazine-1-carboxylate (SM-3, 5 g, 26.88 mmol, 1.0 eq.) in DCM (50 mL, 10 vol), pyridine (5.47 mL, 67.2 mmol, 2.5 eq.) and triphosgene (3.99 g, 13.44 mmol, 0.5 eq.) were added at 0° C. under argon atmosphere. The reaction mixture was allowed to warm up to room temperature and stir for 2 h. Progress of the reaction was monitored by TLC. After completion of the reaction, the reaction mixture was quenched with 1N HCl (˜200 mL) and extracted with DCM (2×200 mL). The combined organic layer was washed with brine solution (200 mL), dried over anhydrous sodium sulfate, filtered and concentrated under reduced pressure to obtain crude tert-butyl 4-(chlorocarbonyl)piperazine-1-carboxylate as a semi-solid. To a second flask charged with(S)-4-ethyl-4,9-dihydroxy-10-((4-methylpiperazin-1-yl)methyl)-1,12-dihydro-14H-pyrano[3′,4′:6,7]-indolizino[1,2-b]quinoline-3,14(4H)-dione (Int-1, 6.3 g, 13.44 mmol, 0.5 eq.) in DCM (25 mL, 10 vol), DIPEA (6.19 mL, 33.6 mmol, 2.5 eq.) and DMAP (327 mg, 2.68 mmol, 0.1 eq.) were added at 0° C. under argon atmosphere. The carbonylchloride above was dissolved in DCM (25 mL) and added to the reaction mixture dropwise at 0° C. under argon atmosphere. The resulting reaction mixture was then allowed to stir at ambient temperature until TLC indicated complete consumption of starting material. The reaction mixture was then concentrated under reduced pressure, diluted with ice cold water (100 mL), filtered the resulting precipitate and dried the crude product, which was purified by flash column (silica gel, 100-200 mesh) eluting with 2-5% MeOH in DCM. The pure fractions were combined and concentrated under reduced pressure to obtain (S)-1-(tert-butyl) 4-(4-ethyl-4-hydroxy-10-((4-methylpiperazin-1-yl)methyl)-3,14-dioxo-3,4,12,14-tetrahydro-1H-pyrano[3′,4′:6,7]-indolizino[1,2-b]quinolin-9-yl)piperazine-1,4-dicarboxylate (Int-2, 1.56 g, 26%) as an off-white solid.

¹H NMR (400 MHz, DMSO-d₆) δ 8.98 (s, 1H), 8.12 (d, J=9.29 Hz, 1H), 7.65 (d, J=9.17 Hz, 1H), 7.34 (s, 1H), 5.75 (s, 1H), 5.42 (s, 2H), 5.30 (s, 2H), 3.66-3.88 (m, 4H), 3.40-3.56 (m, 6H), 2.51-2.57 (m, 2H), 2.46-2.49 (m, 1H), 2.14-2.29 (m, 4H), 1.85-1.91 (m, 1H), 1.82-1.93 (m, 1H), 1.40-1.46 (m, 11H), 0.89 (t, J=7.27 Hz, 3H). LCMS: 689.4 [M+H]⁺.

### Step-3: Preparation of (S)-4-Ethyl-4-hydroxy-10-((4-methylpiperazin-1-yl)methyl)-3,14-dioxo-3,4,12,14-tetrahydro-1H-pyrano[3′,4′:6,7]indolizino[1,2-b]quinolin-9-yl piperazine-1-carboxylate (Int-3)

A stirred solution of (S)-1-(tert-butyl) 4-(4-ethyl-4-hydroxy-10-((4-methylpiperazin-1-yl)methyl)-3,14-dioxo-3,4,12,14-tetrahydro-1H-pyrano[3′,4′:6,7]indolizino[1,2-b]quinolin-9-yl) piperazine-1,4-dicarboxylate (Int-2, 1.5 g, 2.18 mmol, 1.0 eq.) in DCM (15 mL, 10 vol), TFA (1.66 mL, 21.8 mmol, 10 eq.) was added at ambient temperature. The reaction mixture was allowed to stir at ambient temperature until TLC indicated complete consumption of starting material. The reaction mixture was then concentrated under reduced pressure, added saturated sodium bicarbonate solution (100 mL) and the aqueous mixture was extracted with 10% MeOH in DCM (2×250 mL). The combined organic layer was washed with brine solution (200 mL), dried over anhydrous sodium sulfate, filtered, concentrated under reduced pressure and triturated with diethyl ether (100 mL) to provide(S)-4-ethyl-4-hydroxy-10-((4-methylpiperazin-1-yl)methyl)-3,14-dioxo-3,4,12,14-tetrahydro-1H-pyrano[3′,4′:6,7]indolizino[1,2-b]quinolin-9-yl piperazine-1-carboxylate (Int-3, 700 mg, 54%) as an off-white solid.

¹H NMR (400 MHz, DMSO-d₆) δ 8.91-8.99 (m, 1H), 8.04-8.13 (m, 1H), 7.59-7.64 (m, 1H), 7.30-7.35 (m, 1H), 6.48-6.53 (m, 1H), 5.38-5.46 (m, 2H), 5.26-5.33 (m, 2H), 3.81-3.86 (m, 2H), 3.56-3.68 (m, 3H), 3.36-3.42 (m, 2H), 3.12-3.18 (m, 2H), 2.74-2.85 (m, 5H), 2.16-2.34 (m, 4H), 2.07-2.13 (m, 4H), 1.81-1.92 (m, 2H), 0.83-0.91 (m, 3H). LCMS: 589.2 [M+H]⁺.

### Step-4: Preparation of (S)-4-Ethyl-4-hydroxy-10-((4-methylpiperazin-1-yl)methyl)-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate

To a stirred solution of (S)-4-ethyl-4-hydroxy-10-((4-methylpiperazin-1-yl)methyl)-3,14-dioxo-3,4,12,14-tetrahydro-1H-pyrano[3′,4′:6,7]indolizino[1,2-b]quinolin-9-yl piperazine-1-carboxylate (Int-3, 2.0 g, 3.40 mmol, 1.0 eq.) in McOH (20 mL, 10 vol), (8S,11R,13S,14S,17R)-17-acetyl-13-methyl-11-(4-(methyl(6-oxohexyl)amino)-phenyl)-3-oxo-2,3,6,7,8,11,12,13,14,15,16,17-dodecahydro-1H-cyclopenta[a]phenanthren-17-yl acetate (Int-C, 3.8 g, 6.80 mmol, 2.0 eq.) and acetic acid (1 mL, catalytic amount) was added at 0° C. under argon atmosphere. The reaction mixture was allowed to stir at RT for 1 h, and NaCNBH₃ (428 mg, 6.80 mmol, 2.0 eq.) was added at 0° C. under argon atmosphere. The resulting reaction mixture was then allowed to stir at room temperature until TLC indicated complete consumption of starting material. The reaction mixture was then quenched with saturated sodium bicarbonate solution (250 mL), extracted with ethyl acetate (2×500 mL) and the combined organic layer was washed with brine solution (200 mL), dried over anhydrous sodium sulfate, filtered and concentrated under reduced pressure to provide the crude product which was purified by Prep.HPLC (column: spherical-C18, 40 uM, 100A; Mobile Phase A: 0.1% FA in water; Mobile Phase B: acetonitrile; flow rate: 25 mL/min, diluent: THF+DMSO). The pure fractions were lyophilized under reduced pressure to obtain (S)-4-ethyl-4-hydroxy-10-((4-methylpiperazin-1-yl)methyl)-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)-hexyl)piperazine-1-carboxylate (486 mg, 12%) as an off-white solid.

¹H NMR (400 MHz, DMSO-d₆) δ 8.97 (s, 1H), 8.14-8.15 (m, 2H), 8.09-8.13 (m, 1H), 7.63 (d, J=9.26 Hz, 1H), 7.34 (s, 1H), 6.96-7.00 (m, 2H), 6.58-6.62 (m, 3H), 6.48-6.54 (m, 2H), 5.66-5.69 (m, 1H), 5.42-5.44 (m, 2H), 5.28-5.33 (m, 2H), 4.38-4.42 (m, 1H), 4.38-4.42 (m, 1H), 3.82-3.86 (m, 2H), 3.66-3.72 (m, 2H), 3.44-3.50 (m, 4H), 3.22-3.27 (m, 5H), 2.29-2.37 (m, 7H), 2.08-2.18 (m, 11H), 1.42-1.51 (m, 5H), 1.26-1.35 (m, 7H), 0.86-0.91 (m, 3H), 0.22-0.25 (m, 3H). LCMS: 1133.7 [M+H]⁺. HPLC purity 91.5%.

### Example S6. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(7-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)heptyl)piperazine-1-carboxylate (Compound No. 6)

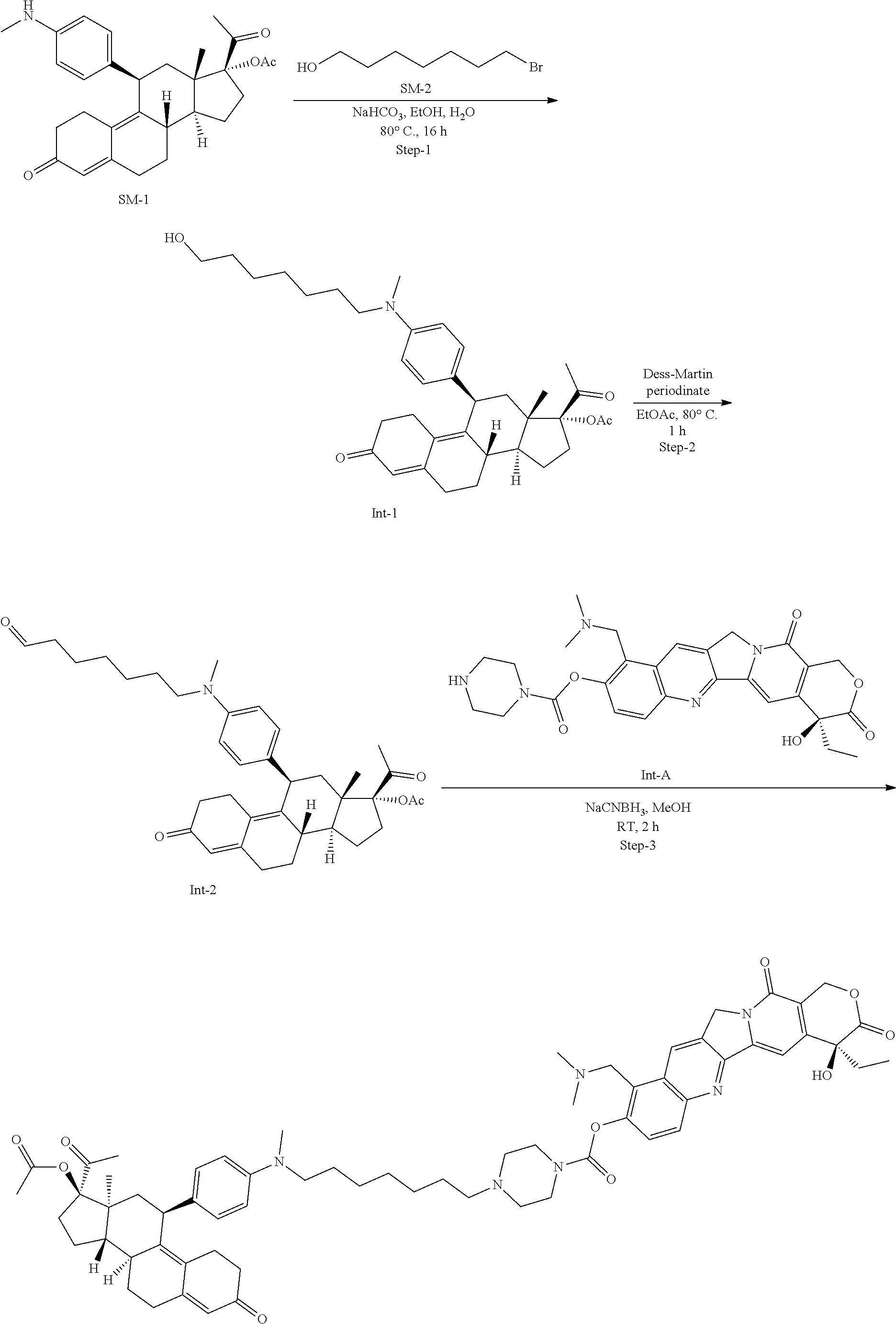

### Step-1: Preparation of (8S,11R,13S,14S,17R)-17-Acetyl-11-(4-((7-hydroxyheptyl)(methyl)amino)phenyl)-13-methyl-3-oxo-2,3,6,7,8,11,12,13,14,15,16,17-dodecahydro-1H-cyclopenta[a]phenanthren-17-yl Acetate (Int-1)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (SM-1, 5.0 g, 10.84 mmol, 1.0 eq.) in EtOH (50 mL, 10 vol) and H₂O (25 mL, 5 vol), 7-bromoheptan-1-ol (SM-2, 10.56 g, 54.2 mmol, 5.0 eq.) and NaHCO₃ (2.73 g, 32.52 mmol, 3.0 eq.) was added at 0° C. under argon atmosphere. The resulting reaction mixture was allowed to stir at 80° C. until TLC indicated complete consumption of starting material. The reaction mixture was then diluted with ice cold water (250 mL), extracted with EtOAc (2×500 mL), combined organic layer washed with brine solution (200 mL), dried over anhydrous sodium sulfate, filtered and concentrated under reduced pressure. The crude obtained was purified by flash column (silica gel, 100-200 mesh) eluting with 20-50% EtOAc in hexane. The pure fractions were combined and concentrated under reduced pressure to obtain (8S,11R,13S,14S,17R)-17-acetyl-11-(4-((7-hydroxyheptyl)(methyl)amino)phenyl)-13-methyl-3-oxo-2,3,6,7,8,11,12,13,14,15,16,17-dodecahydro-1H-cyclopenta[a]phenanthren-17-yl acetate (Int-1, 2 g, 32%) as an off-white solid.

H NMR (400 MHz, DMSO-d₆) δ 6.97 (d, J=8.31 Hz, 2H), 6.57 (d, J=8.31 Hz, 2H), 5.64-5.70 (m, 1H), 4.36-4.41 (m, 1H), 4.27-4.32 (m, 1H), 3.33-3.38 (m, 3H), 3.18-3.26 (m, 2H), 2.78-2.82 (m, 3H), 2.64-2.77 (m, 2H), 2.54-2.64 (m, 2H), 2.30-2.41 (m, 1H), 2.19-2.24 (m, 1H), 2.11-2.15 (m, 1H), 2.06-2.11 (m, 3H), 1.97-2.03 (m, 4H), 1.85-1.93 (m, 1H), 1.64-1.75 (m, 2H), 1.35-1.50 (m, 6H), 1.19-1.32 (m, 8H), 0.17-0.26 (m, 3H). LCMS: 576.96 [M+H]⁺.

### Step-2: Preparation of (8S,11R,13S,14S,17R)-17-Acetyl-13-methyl-11-(4-(methyl(7-oxoheptyl)amino)phenyl)-3-oxo-2,3,6,7,8,11,12,13,14,15,16,17-dodecahydro-1H-cyclopenta[a]phenanthren-17-yl Acetate (Int-2)

To a stirred solution of (8S,11R,13S,14S,17R)-17-acetyl-11-(4-((7-hydroxyheptyl)(methyl)amino)-phenyl)-13-methyl-3-oxo-2,3,6,7,8,11,12,13,14,15,16,17-dodecahydro-1H-cyclopenta[a]phenanthren-17-yl acetate (Int-1, 3 g, 5.2 mmol, 1.0 eq.) in EtOAc (60 mL, 20 vol), DMP (4.4 g, 10.4 mmol, 2.0 eq.) was added at 0° C. under argon atmosphere. The resulting reaction mixture was stirred at 80° C. until TLC indicated complete consumption of starting material. The reaction mixture was then quenched with a mixture of Na₂S₂O₃ and saturated sodium bicarbonate solution (1:1, 250 mL), extracted with EtOAc (2×500 mL), combined organic layer washed with brine solution (200 mL), dried over anhydrous sodium sulfate, filtered and concentrated under reduced pressure to obtain crude (8S,11R,13S,14S,17R)-17-acetyl-13-methyl-11-(4-(methyl(7-oxoheptyl)amino)phenyl)-3-oxo-2,3,6,7,8,11,12,13,14,15,16,17-dodecahydro-1H-cyclopenta[a]phenanthren-17-yl acetate (Int-2, 3 g, crude) as an off-white solid, which was used in the next step without further purification.

¹H NMR (400 MHz, DMSO-d₆) δ 9.65 (s, 1H), 6.97 (d, J=7.82 Hz, 1H), 6.58 (d, J=8.80 Hz, 1H), 5.67 (s, 1H), 4.39 (d, J=6.36 Hz, 1H), 4.03 (q, J=6.85 Hz, 1H), 3.17-3.26 (m, 2H), 2.65-2.84 (m, 4H), 2.52-2.65 (m, 2H), 2.28-2.44 (m, 3H), 2.05-2.25 (m, 6H), 1.84-2.04 (m, 7H), 1.62-1.76 (m, 2H), 1.34-1.54 (m, 5H), 1.21-1.31 (m, 5H), 1.15-1.20 (m, 2H), 0.21-0.26 (m, 3H). LCMS: 574.72 [M+H]⁺.

### Step-3: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(7-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)heptyl)piperazine-1-carboxylate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate (Int-A, 3.0 g, 5.62 mmol, 1.0 eq.) in MeOH (30 mL, 10 vol), (8S,11R,13S,14S,17R)-17-acetyl-13-methyl-11-(4-(methyl(7-oxoheptyl)amino)phenyl)-3-oxo-2,3,6,7,8,11,12,13,14,15,16,17-dodecahydro-1H-cyclopenta[a]phenanthren-17-yl acetate (Int-2, 3.2 g, 5.62 mmol, 1.0 eq.) and acetic acid (1 mL, catalytic amount) were added at 0° C. under argon atmosphere. The reaction mixture was allowed to stir at RT for 1 h., after which NaCNBH₃ (705 mg, 11.24 mmol, 2.0 eq.) was added at 0° C. under argon atmosphere. The resulting reaction mixture was then allowed to stir at room temperature until TLC indicated complete consumption of starting material. The reaction mixture was then quenched with saturated sodium bicarbonate solution (250 mL), extracted with ethyl acetate (2×500 mL), combined organic layer washed with brine solution (200 mL), dried over anhydrous sodium sulfate, filtered and concentrated under reduced pressure. The crude obtained was purified by Prep HPLC. (column: spherical-C18, 40 uM, 100A; Mobile Phase A: 0.1% FA in water; Mobile Phase B: acetonitrile; flow rate: 25 mL/min, diluent: THF+DMSO). The pure fractions were combined and lyophilized under reduced pressure to obtain (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(7-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)heptyl)piperazine-1-carboxylate (2.35 g, 38%) as an off-white solid.

¹H NMR (400 MHz, DMSO-d₆) δ 8.96-9.00 (m, 1H), 8.09-8.16 (m, 1H), 7.60-7.65 (m, 1H), 7.32-7.36 (m, 1H), 6.97-7.01 (m, 2H), 6.57-6.62 (m, 2H), 6.49-6.51 (m, 1H), 5.66-5.70 (m, 1H), 5.42-5.44 (m, 2H), 5.30-5.32 (m, 2H), 4.37-4.42 (m, 1H), 3.80-3.87 (m, 2H), 3.64-3.73 (m, 2H), 3.42-3.51 (m, 2H), 3.24-3.25 (m, 3H), 2.80-2.84 (m, 4H), 2.41-2.43 (m, 2H), 2.31-2.34 (m, 4H), 2.08-2.15 (m, 12H), 1.99-2.01 (m, 5H), 1.85-1.92 (m, 4H), 1.65-1.73 (m, 3H), 1.41-1.51 (m, 5H), 1.27-1.34 (m, 6H), 0.86-0.91 (m, 4H), 0.23-0.26 (m, 3H). LCMS: 1091.1 [M+H]⁺.

### Example S7. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(8-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)octyl)piperazine-1-carboxylate (Compound No. 7)

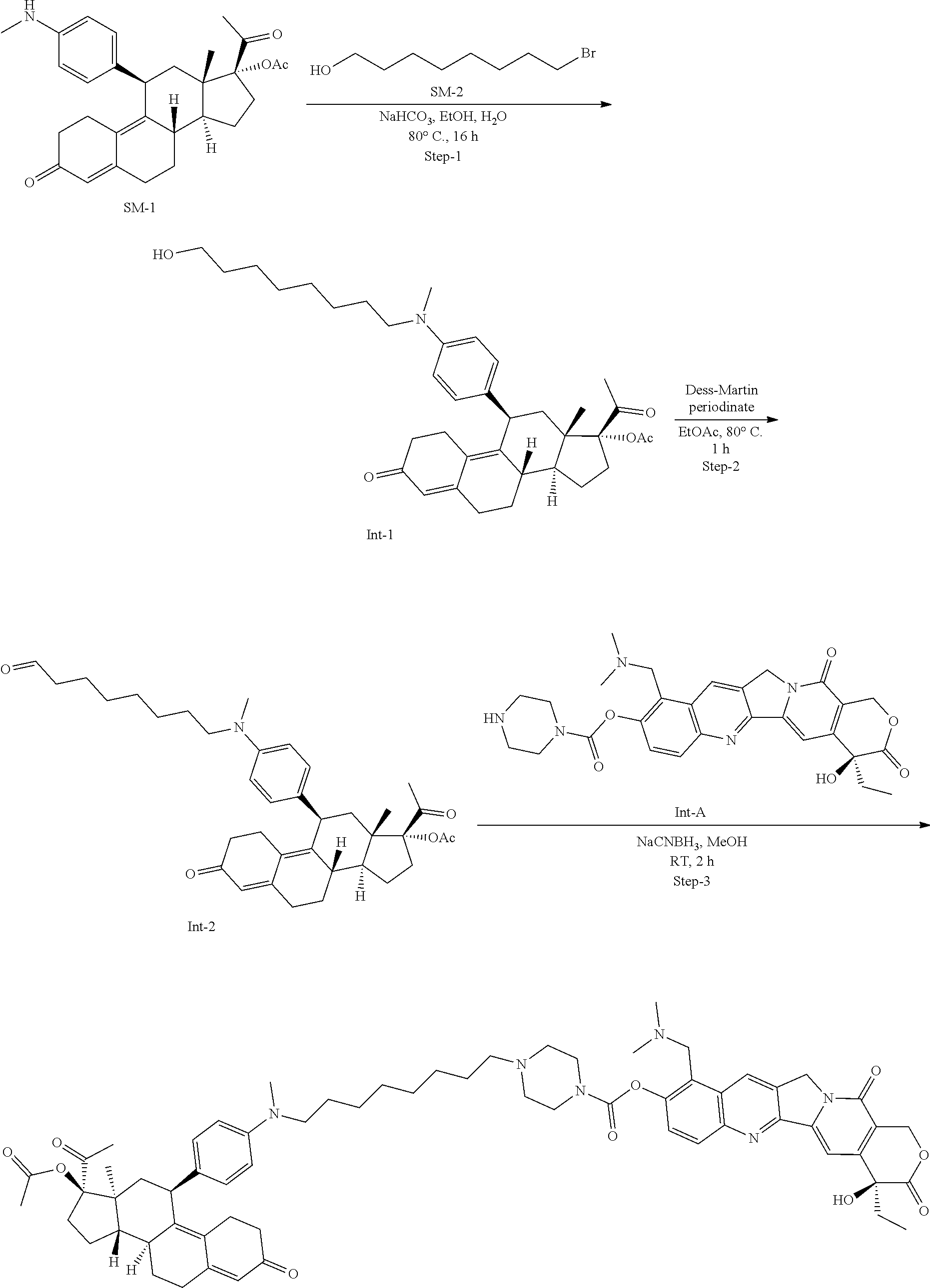

### Step-1: Preparation of (8S,11R,13S,14S,17R)-17-Acetyl-11-(4-((8-hydroxyoctyl)(methyl)amino)phenyl)-13-methyl-3-oxo-2,3,6,7,8,11,12,13,14,15,16,17-dodecahydro-1H-cyclopenta[a]phenanthren-17-yl Acetate (Int-1)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (SM-1, 10.0 g, 21.69 mmol, 1.0 eq.) in EtOH (100 mL, 10 vol) and H₂O (50 mL, 5 vol), 8-bromooctan-1-ol (SM-2, 22.66 g, 108.45 mmol, 5.0 eq.) and NaHCO₃ (5.46 g, 61.07 mmol, 3.0 eq.) were added at 0° C. under argon atmosphere. The resulting reaction mixture was allowed to stir at 80° C. until TLC indicated complete consumption of starting material. The reaction mixture was then diluted with ice cold water (250 mL), extracted with EtOAc (2×500 mL), combined organic layer washed with brine solution (200 mL), dried over anhydrous sodium sulfate, filtered and concentrated under reduced pressure. The crude obtained was purified by flash column (silica gel, 100-200 mesh) eluting with 20-50% EtOAc in hexane. The pure fractions were combined and concentrated under reduced pressure to obtain (8S,11R,13S,14S,17R)-17-acetyl-11-(4-((8-hydroxyoctyl)(methyl)amino)phenyl)-13-methyl-3-oxo-2,3,6,7,8,11,12,13,14,15,16,17-dodecahydro-1H-cyclopenta[a]phenanthren-17-yl acetate (Int-1, 5 g, 39%) as an off-white solid.

¹H NMR (400 MHz, DMSO-d₆) δ 6.97 (d, J=7.83 Hz, 2H), 6.57 (d, J=7.83 Hz, 2H), 5.74-5.77 (m, 1H), 5.65-5.69 (m, 1H), 4.36-4.42 (m, 1H), 4.26-4.35 (m, 2H), 3.33-3.40 (m, 6H), 3.18-3.26 (m, 1H), 2.80 (s, 3H), 2.67-2.77 (m, 1H), 2.54-2.64 (m, 2H), 2.31-2.41 (m, 1H), 2.12-2.24 (m, 2H), 2.10 (s, 3H), 1.89-2.04 (m, 3H), 1.61-1.78 (m, 1H), 1.35-1.45 (m, 6H), 1.21-1.30 (m, 10H), 0.23 (s, 3H). LCMS: 590.5 [M+H]⁺.

### Step-2: Preparation of (8S,11R,13S,14S,17R)-17-Acetyl-13-methyl-11-(4-(methyl(8-oxooctyl)amino)phenyl)-3-oxo-2,3,6,7,8,11,12,13,14,15,16,17-dodecahydro-1H-cyclopenta[a]phenanthren-17-yl Acetate (Int-2)

To a stirred solution of ((8S,11R,13S,14S,17R)-17-acetyl-11-(4-((8-hydroxyoctyl)(methyl)amino)phenyl)-13-methyl-3-oxo-2,3,6,7,8,11,12,13,14,15,16,17-dodecahydro-1H-cyclopenta[a]phenanthren-17-yl acetate (Int-1, 4 g, 6.79 mmol, 1.0 eq.) in EtOAc (80 mL, 20 vol), DMP (5.75 g, 13.5 mmol, 2.0 eq.) was added at 0° C. under argon atmosphere. The resulting reaction mixture was stirred at 80° C. until TLC indicated complete consumption of starting material. The reaction mixture was then quenched with a mixture of Na₂S₂O₃ and saturated sodium bicarbonate solution (1:1, 250 mL), extracted with EtOAc (2×500 mL). The combined organic layer was washed with brine solution (200 mL), dried over anhydrous sodium sulfate, filtered and concentrated under reduced pressure to obtain (8S,11R,13S,14S,17R)-17-acetyl-13-methyl-11-(4-(methyl(8-oxooctyl)amino)phenyl)-3-oxo-2,3,6,7,8,11,12,13,14,15,16,17-dodecahydro-1H-cyclopenta[a]phenanthren-17-yl acetate (Int-2, 4 g, crude) as an off-white solid, which was used in the next step without further purification.

¹H NMR (400 MHz, DMSO-d₆) δ 9.66 (s, 1H), 6.97 (d, J=7.82 Hz, 1H), 6.57 (d, J=7.82 Hz, 1H), 5.66-5.70 (m, 1H), 4.35-4.45 (m, 1H), 3.99-4.06 (m, 2H), 3.21 (d, J=6.36 Hz, 1H), 2.79-2.82 (m, 2H), 2.66-2.78 (m, 2H), 2.54-2.64 (m, 2H), 2.37-2.44 (m, 3H), 2.29-2.36 (m, 1H), 2.18-2.26 (m, 1H), 2.09-2.17 (m, 4H), 1.97-2.01 (m, 5H), 1.85-1.92 (m, 1H), 1.66-1.74 (m, 1H), 1.41-1.56 (m, 6H), 1.22-1.28 (m, 8H), 1.17 (t, J=7.09 Hz, 3H), 0.20-0.25 (m, 2H). LCMS: 588.66 [M+H]⁺.

### Step-3: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(8-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)octyl)piperazine-1-carboxylate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate (Int-A, 4.0 g, 7.50 mmol, 1.0 eq.) in MeOH (40 mL, 10 vol), (8S,11R,13S,14S,17R)-17-acetyl-13-methyl-11-(4-(methyl(8-oxooctyl)amino)-phenyl)-3-oxo-2,3,6,7,8,11,12,13,14,15,16,17-dodecahydro-1H-cyclopenta[a]phenanthren-17-yl acetate (Int-2, 4.4 g, 7.50 mmol, 1.0 eq.) and acetic acid (1 mL, catalytic amount) were added at 0° C. under argon atmosphere. The reaction mixture was allowed to stir at RT for 1 h., after which time NaCNBH₃ (945 mg, 15.0 mmol, 2.0 eq.) was added at 0° C. under argon atmosphere. The resulting reaction mixture was then allowed to stir at room temperature until TLC indicated complete consumption of starting material, quenched with saturated sodium bicarbonate solution (250 mL) and extracted with ethyl acetate (2×500 mL). The combined organic layer was washed with brine solution (200 mL), dried over anhydrous sodium sulfate, filtered and concentrated under reduced pressure. The crude obtained was purified by Prep. HPLC (column: spherical-C18, 40 uM, 100A; Mobile Phase A: 0.1% FA in water; Mobile Phase B: acetonitrile; flow rate: 25 mL/min, diluent: THF+DMSO). The pure fractions were combined and lyophilized under reduced pressure to obtain (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(8-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)octyl)piperazine-1-carboxylate (1.19 g, 14%) as an off-white solid.

¹H NMR (400 MHz, DMSO-d₆) δ 8.92-8.97 (m, 1H), 8.08-8.15 (m, 2H), 7.61-7.66 (m, 1H), 7.33-7.35 (m, 1H), 6.96-7.00 (m, 2H), 6.57-6.61 (m, 2H), 6.49-6.54 (m, 1H), 5.66-5.69 (m, 1H), 5.42-5.45 (m, 2H), 5.30-5.33 (m, 2H), 4.38-4.41 (m, 1H), 3.67-3.78 (m, 4H), 3.44-3.51 (m, 2H), 3.20-3.27 (m, 4H), 2.82 (s, 3H), 2.39-2.46 (m, 3H), 2.31-2.37 (m, 3H), 2.19-2.21 (m, 7H), 2.08-2.10 (m, 4H), 1.98-2.01 (m, 4H), 1.98-2.02 (m, 4H), 1.83-1.93 (m, 3H), 1.62-1.78 (m, 1H), 1.42-1.51 (m, 5H), 1.28 (br s, 10H), 0.86-0.92 (m, 4H), 0.24-0.25 (m, 3H). LCMS: 1105.5 [M+H]⁺.

### Example S8. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(5-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino) pentyl)piperazine-1-carboxylate (Compound No. 8)

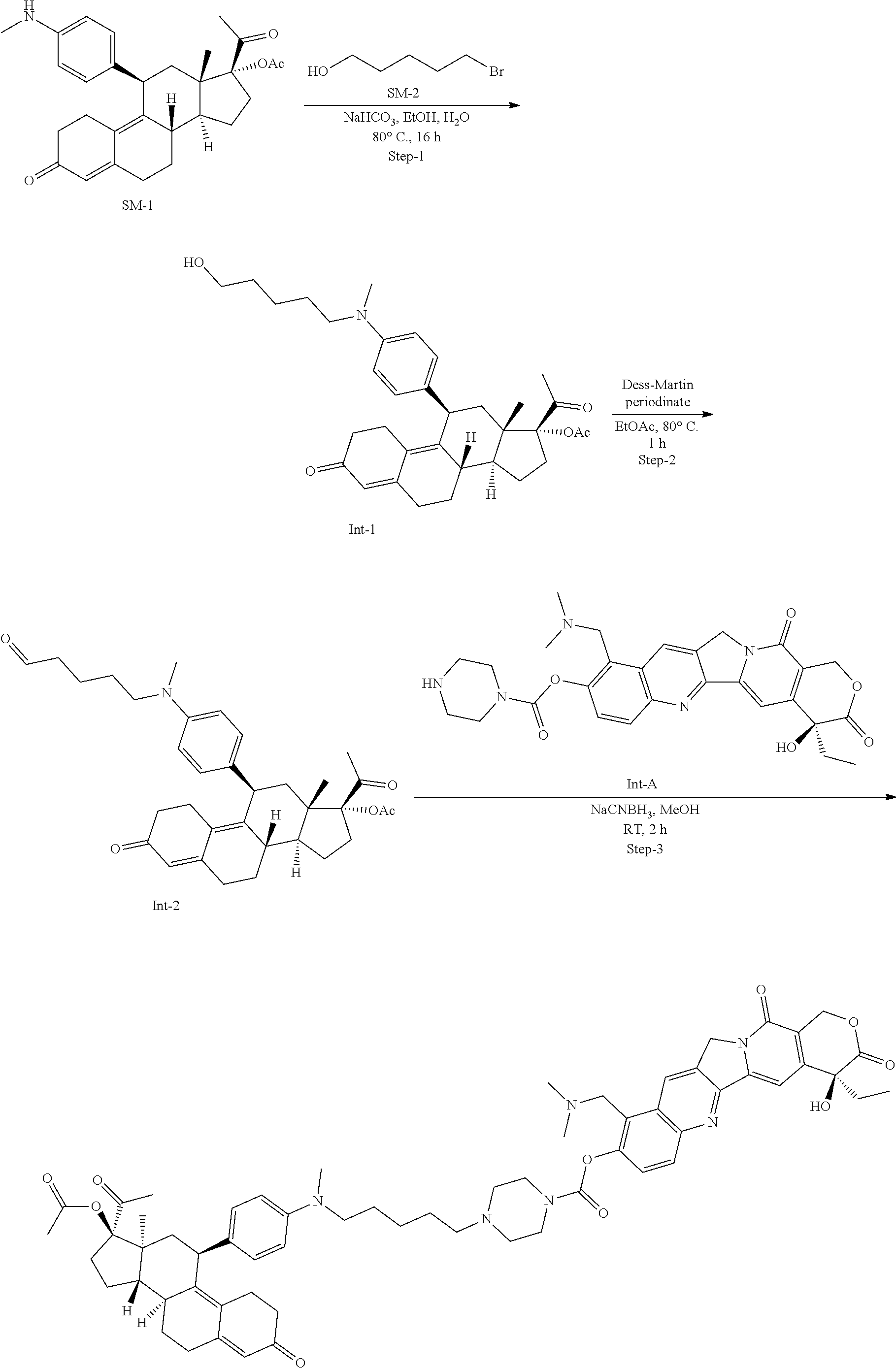

### Step-1: Preparation of (8S,11R,13S,14S,17R)-17-Acetyl-11-(4-((5-hydroxypentyl)(methyl)amino)phenyl)-13-methyl-3-oxo-2,3,6,7,8,11,12,13,14,15,16,17-dodecahydro-1H-cyclopenta[a]phenanthren-17-yl Acetate (Int-1)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (SM-1, 9.0 g, 19.52 mmol, 1.0 eq.) in EtOH (90 mL, 10 vol) and H₂O (45 mL, 5 vol), 5-bromopentan-1-ol (SM-2, 16.29 g, 97.6 mmol, 5.0 eq.) and NaHCO₃ (4.91 g, 58.56 mmol, 3.0 eq.) were added at 0° C. under argon atmosphere. The resulting reaction mixture was allowed to stir at 80° C. until TLC indicated complete consumption of starting material. The reaction mixture was then diluted with ice cold water (250 mL), extracted with EtOAc (2×500 mL), combined organic layer washed with brine solution (200 mL), dried over anhydrous sodium sulfate, filtered and concentrated under reduced pressure. The obtained crude was purified by flash column (silica gel, 100-200 mesh) eluting with 20-50% EtOAc in hexane. The pure fractions were combined and concentrated under reduced pressure to obtain (8S,11R,13S,14S,17R)-17-acetyl-11-(4-((5-hydroxypentyl)(methyl)amino)phenyl)-13-methyl-3-oxo-2,3,6,7,8,11,12,13,14,15,16,17-dodecahydro-1H-cyclopenta[a]phenanthren-17-yl acetate (Int-1, 2.2 g, 20%) as an off-white solid.

¹H NMR (400 MHz, DMSO-d₆) δ 6.98 (d, J=8.31 Hz, 2H), 6.55-6.60 (m, 2H), 5.67 (s, 1H), 4.29-4.42 (m, 2H), 4.00-4.07 (m, 1H), 3.36 (q, J=5.87 Hz, 2H), 3.19-3.24 (m, 2H), 2.81 (s, 3H), 2.66-2.77 (m, 2H), 2.54-2.64 (m, 2H), 2.31-2.40 (m, 1H), 2.19-2.24 (m, 1H), 2.12-2.18 (m, 2H), 2.07-2.10 (m, 3H), 1.94-2.02 (m, 5H), 1.86-1.94 (m, 1H), 1.65-1.75 (m, 2H), 1.39-1.49 (m, 5H), 1.23-1.33 (m, 2H), 1.17 (t, J=7.09 Hz, 1H), 0.23 (s, 3H). LCMS: 548.4 [M+H]⁺.

### Step-2: Preparation of (8S,11R,13S,14S,17R)-17-Acetyl-13-methyl-11-(4-(methyl(5-oxopentyl)amino)phenyl)-3-oxo-2,3,6,7,8,11,12,13,14,15,16,17-dodecahydro-1H-cyclopenta[a]phenanthren-17-yl Acetate (Int-2)

To a stirred solution of (8S,11R,13S,14S,17R)-17-acetyl-11-(4-((5-hydroxypentyl)(methyl)amino)-phenyl)-13-methyl-3-oxo-2,3,6,7,8,11,12,13,14,15,16,17-dodecahydro-1H-cyclopenta[a]phenanthren-17-yl acetate (Int-1, 2 g, 3.65 mmol, 1.0 eq.) in EtOAc (40 mL, 20 vol). DMP (3.09 g, 7.3 mmol, 2.0 eq.) was added at 0° C. under argon atmosphere. The resulting reaction mixture was allowed to stir at 80° C. until TLC indicated complete consumption of starting material. The reaction mixture was then quenched with a mixture of Na₂S₂O₃ and saturated sodium bicarbonate solution (1:1, 250 mL), extracted with EtOAc (2×500 mL) and the combined organic layer was washed with brine solution (200 mL), dried over anhydrous sodium sulfate, filtered and concentrated under reduced pressure to obtain (8S,11R,13S,14S,17R)-17-acetyl-13-methyl-11-(4-(methyl(5-oxopentyl)amino)phenyl)-3-oxo-2,3,6,7,8,11,12,13,14,15,16,17-dodecahydro-1H-cyclopenta[a]phenanthren-17-yl acetate (Int-2, 2 g, crude) as an off-white solid, which was used in the next step without further purification.

¹H NMR (400 MHz, DMSO-d₆) δ 9.64 (s, 1H), 6.98 (d, J=8.31 Hz, 2H), 6.59 (d, J=8.31 Hz, 2H), 5.67 (s, 1H), 4.36-4.43 (m, 1H), 3.98-4.07 (m, 1H), 3.19-3.27 (m, 2H), 2.81 (s, 3H), 2.53-2.78 (m, 5H), 2.29-2.49 (m, 3H), 2.12-2.25 (m, 3H), 2.10 (s, 3H), 1.84-2.05 (m, 7H), 1.59-1.80 (m, 2H), 1.42-1.56 (m, 3H), 1.17 (t, J=7.09 Hz, 1H), 0.23 (s, 3H). LCMS: 546.68 [M+H]⁺.

### Step-3: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(5-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino) pentyl)piperazine-1-carboxylate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate (Int-A, 4.0 g, 7.50 mmol, 1.0 eq.) in MeOH (40 mL, 10 vol), (8S,11R,13S,14S,17R)-17-acetyl-13-methyl-11-(4-(methyl(5-oxopentyl)amino)phenyl)-3-oxo-2,3,6,7,8,11,12,13,14,15,16,17-dodecahydro-1H-cyclopenta[a]phenanthren-17-yl acetate (Int-2, 4.1 g, 7.50 mmol, 1.0 eq.) and acetic acid (1 mL, catalytic amount) were added at 0° C. under argon atmosphere. The reaction mixture was allowed to stir at RT for 1 h. and NaCNBH₃ (945 mg, 15.0 mmol, 2.0 eq.) was added at 0° C. under argon atmosphere. The resulting reaction mixture was then allowed to stir at room temperature until TLC indicated complete consumption of starting material. The reaction mixture was then quenched with saturated sodium bicarbonate solution (250 mL) and extracted with ethyl acetate (2×500 mL). The combined organic layer was washed with brine solution (200 mL), dried over anhydrous sodium sulfate, filtered and concentrated under reduced pressure. The crude obtained was purified by Prep. HPLC. (column: spherical-C18, 40 uM, 100A; Mobile Phase A: 0.1% FA in water; Mobile Phase B: acetonitrile; flow rate: 25 mL/min, diluent: THF+DMSO). The pure fractions were combined and lyophilized under reduced pressure to obtained(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(5-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)-pentyl)piperazine-1-carboxylate (1.12 g, 14%) as an off-white solid.

¹H NMR (400 MHz, DMSO-d₆) δ ppm 8.94 (s, 1H), 8.16 (s, 2H), 8.11 (d, J=9.29 Hz, 1H), 7.63 (d, J=8.80 Hz, 1H), 7.34 (s, 1H), 6.99 (d, J=7.82 Hz, 2H), 6.60 (d, J=7.83 Hz, 2H), 5.66-5.69 (m, 1H), 5.43 (s, 2H), 5.31 (br s, 2H), 4.37-4.42 (m, 1H), 3.76 (br s, 2H), 3.67-3.72 (m, 2H), 3.44-3.48 (m, 3H), 3.21-3.30 (m, 5H), 2.83 (s, 3H), 2.52-2.79 (m, 5H), 2.29-2.44 (m, 6H), 2.20 (s, 6H), 2.10 (s, 3H), 2.00 (s, 3H), 1.80-1.94 (m, 3H), 1.64-1.77 (m, 1H), 1.39-1.57 (m, 4H), 1.24-1.38 (m, 3H), 0.89 (t, J=6.85 Hz, 3H), 0.21-0.27 (m, 3H). LCMS: 1063.2 [M+H]⁺.

### Example S9. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-(4-(3-(4-cyano-3-(trifluoromethyl)phenyl)-5,5-dimethyl-4-oxo-2-thioxoimidazolidin-1-yl)-2-fluorobenzamido)hexanoyl)piperazine-1-carboxylate (Compound No. 9)

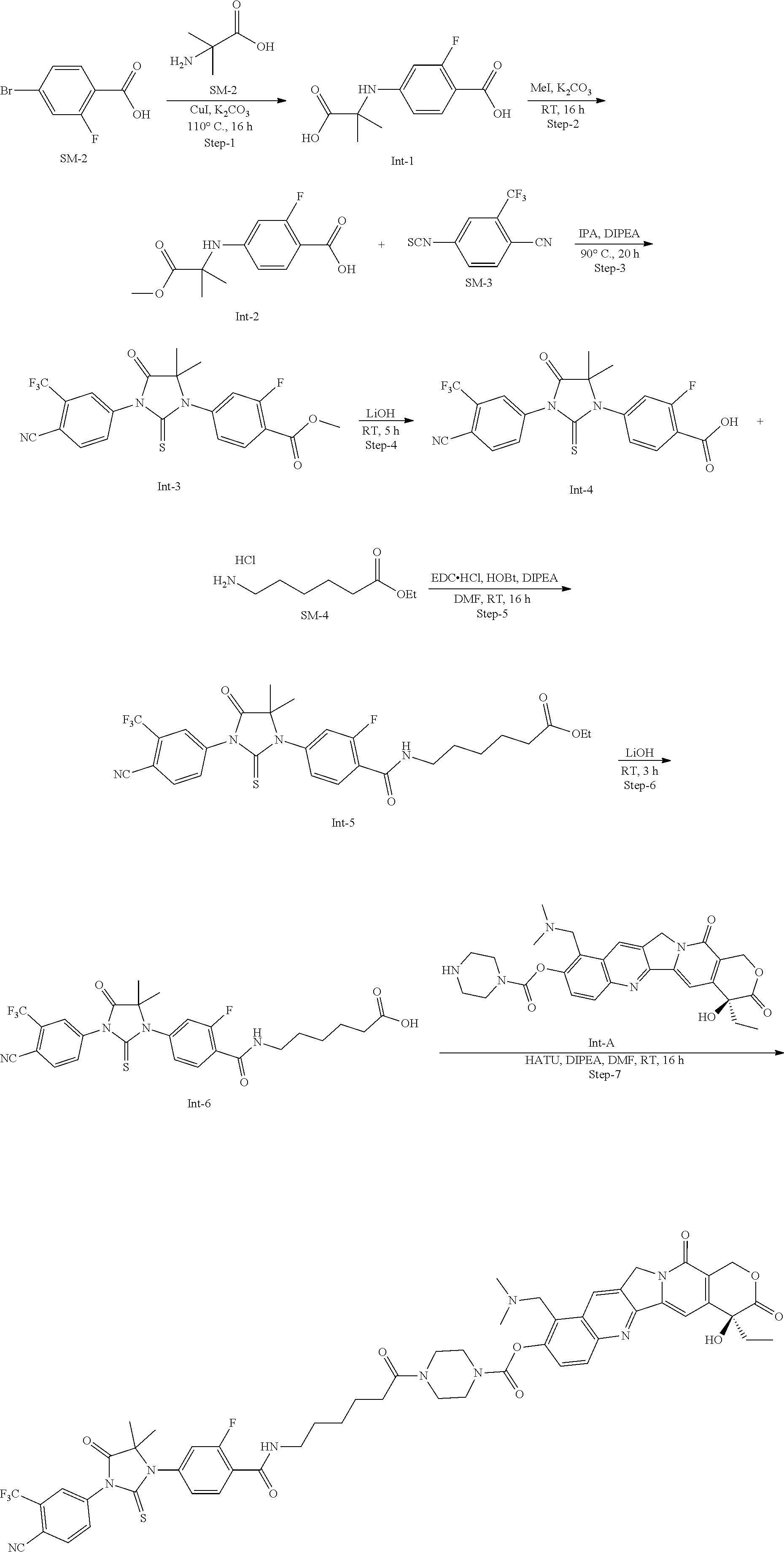

### Step-1: Preparation of 4-((2-Carboxypropan-2-yl)amino)-2-fluorobenzoic acid (Int-1)

To a stirred solution of 4-bromo-2-fluorobenzoic acid (SM-1, 10.0 g, 45.66 mmol, 1.0 eq.) in DMF (100 mL, 10 vol) and water (10 mL, 1 vol), 2-amino-2-methylpropanoic acid (SM-2, 14.1 g, 136.98 mmol, 3.0 eq.), N,N-dimethylglycine (2.35 g, 22.83 mmol, 0.5 eq.), K₂CO₃ (31.5 g, 228.3 mmol, 5.0 eq.), Cu powder (575 mg, 9.13 mmol, 0.2 eq.) and copper iodide (1.73 g, 9.13 mmol, 0.2 eq.) were added at room temperature. The reaction mixture was allowed to stir at 110° C. until TLC indicated complete consumption of starting material. The reaction mixture was then diluted with ice cold water (500 mL) and acidified with 6N HCl to pH˜4. The resulting aqueous solution was then extracted with ethyl acetate (2×1 L) and the combined organic layer was washed with brine solution (300 mL), dried over sodium sulfate, filtered, concentrated under reduced pressure and recrystallized with DCM to obtain 4-((2-carboxypropan-2-yl)amino)-2-fluorobenzoic acid (Int-1, 6.2 g, 56%) as an off-white solid.

¹H NMR (400 MHz, DMSO-d₆) δ 12.52 (br s, 2H), 7.59 (t, J=8.79 Hz, 1H), 6.96 (s, 1H), 6.33 (dd, J=8.79, 1.85 Hz, 1H), 6.15 (dd, J=14.57, 1.62 Hz, 1H), 1.44 (s, 6H). LCMS: 242.15 [M+H]⁺.

### Step-2: Preparation of Methyl 2-Fluoro-4-((1-methoxy-2-methyl-1-oxopropan-2-yl)amino)benzoate (Int-2)

To a stirred solution of 4-((2-carboxypropan-2-yl)amino)-2-fluorobenzoic acid (Int-1, 6.2 g, 25.72 mmol, 1.0 eq.) in DMF (70 mL, 10 vol), Mel (3.1 mL, 51.45 mmol, 2.0 eq.), K₂CO₃ (53.1 g, 385.5 mmol, 15.0 eq.) were added at room temperature. The reaction mixture was allowed to stir at ambient temperature until TLC indicated complete consumption of starting material. The reaction mixture was then diluted with ice cold water (500 mL), filtered and dried to obtain methyl 2-fluoro-4-((1-methoxy-2-methyl-1-oxopropan-2-yl)amino)benzoate (Int-2, 4.82 g, 69%) as an off-white solid.

¹H NMR (400 MHz, DMSO-d₆) δ 7.61 (t, J=8.80 Hz, 1H), 7.11 (s, 1H), 6.29 (dd, J=8.80, 2.45 Hz, 1H), 6.14 (dd, J=14.67, 1.96 Hz, 1H), 3.74 (s, 3H), 3.63 (s, 3H), 1.48 (s, 6H). LCMS: 270.10 [M+H]⁺.

### Step-3: Preparation of Methyl 4-(3-(4-Cyano-3-(trifluoromethyl)phenyl)-5,5-dimethyl-4-oxo-2-thioxoimidazolidin-1-yl)-2-fluorobenzoate (Int-3)

To a stirred solution of methyl 2-fluoro-4-((1-methoxy-2-methyl-1-oxopropan-2-yl)amino)benzoate (Int-2, 4.8 g, 17.84 mmol, 1.0 eq.) in DMSO (7.2 mL, 1.5 vol), 4-isothiocyanato-2-(trifluoromethyl)benzonitrile (SM-3, 8.5 g, 37.59 mmol, 2.1 eq.) was added at room temperature. The reaction mixture was allowed to stir at 90° C. until TLC indicated complete consumption of starting material. The reaction mixture was then diluted with ice cold water (500 mL), extracted with ethyl acetate (2×1 L) and the combined organic layer was washed with brine solution (300 mL), dried over sodium sulfate, filtered and concentrated under reduced pressure. The crude obtained was purified by column chromatography (silica gel, 100-200 mesh) eluting with 20-30% ethyl acetate in hexane. The pure fractions were combined and concentrated under reduced pressure to provide methyl 4-(3-(4-cyano-3-(trifluoromethyl)phenyl)-5,5-dimethyl-4-oxo-2-thioxoimidazolidin-1-yl)-2-fluorobenzoate (Int-3, 5 g, 60%) as an off-white solid.

¹H NMR (400 MHz, DMSO-d₆) δ 8.41 (d, J=8.31 Hz, 1H), 8.29 (d, J=1.47 Hz, 1H), 8.06-8.11 (m, 2H), 7.51 (dd, J=11.25, 1.96 Hz, 1H), 7.41 (dd, J=8.31, 1.96 Hz, 1H), 3.90 (s, 3H), 1.55 (s, 6H). LCMS: 466.50 [M+H]⁺.

### Step-4: Preparation of 4-(3-(4-Cyano-3-(trifluoromethyl)phenyl)-5,5-dimethyl-4-oxo-2-thioxoimidazolidin-1-yl)-2-fluorobenzoic Acid (Int-4)

To a flask charged with methyl 4-(3-(4-cyano-3-(trifluoromethyl)phenyl)-5,5-dimethyl-4-oxo-2-thioxoimidazolidin-1-yl)-2-fluorobenzoate (Int-3, 3 g, 6.45 mmol, 1.0 eq.) in McOH:THF:H₂O (1:1:1, 30 mL, 10 vol), LiOH (810 mg, 19.35 mmol, 3.0 eq.) was added at ambient temperature under argon atmosphere. The reaction mixture was allowed to stir at ambient temperature until TLC indicated complete consumption of starting material. The reaction mixture was concentrated under reduced pressure, diluted with water (10 mL), acidified with citric acid to pH˜3, filtered the resulting solid and dried to obtain 4-(3-(4-cyano-3-(trifluoromethyl)phenyl)-5,5-dimethyl-4-oxo-2-thioxoimidazolidin-1-yl)-2-fluorobenzoic acid (Int-4, 2.6 g, 89%) as an off-white solid.

¹H NMR (400 MHz, DMSO-d₆) δ 8.40 (d, J=8.31 Hz, 1H), 8.29 (s, 1H), 8.08 (d, J=8.31 Hz, 1H), 7.94 (t, J=8.07 Hz, 1H), 7.36 (d, J=10.76 Hz, 1H), 7.29 (d, J=8.31 Hz, 1H), 1.54 (s, 6H). LCMS: 452.20 [M+H]⁺.

### Step-5: Preparation of Ethyl 6-(4-(3-(4-Cyano-3-(trifluoromethyl)phenyl)-5,5-dimethyl-4-oxo-2-thioxoimidazolidin-1-yl)-2-fluorobenzamido)hexanoate (Int-5)

To a stirred solution of 4-(3-(4-cyano-3-(trifluoromethyl)phenyl)-5,5-dimethyl-4-oxo-2-thioxoimidazolidin-1-yl)-2-fluorobenzoic acid (Int-4, 2.6 g, 5.76 mmol, 1.0 eq.) in DMF (26 mL, 10 vol), ethyl 6-aminohexanoate·hydrogen chloride (SM-4, 1.68 g, 8.64 mmol, 1.5 eq.), EDC·HCl (1.65 g, 8.64 mmol, 1.5 eq.), HOBt (1.67 g, 8.64 mmol, 1.5 eq.), DMAP (70 mg, 0.57 mmol, 0.1 eq.) and DIPEA (3.18 mL, 17.28 mmol, 3.0 eq.) were added at room temperature. The reaction mixture was allowed to stir at ambient temperature until TLC indicated complete consumption of starting material, diluted with ice cold water (100 mL) and extracted with ethyl acetate (2×250 mL). The combined organic layer was washed with brine solution (200 mL), dried over sodium sulfate, filtered, concentrated under reduced pressure and purified by flash column chromatography (silica gel, 100-200 mesh) eluting with 40-60% ethyl acetate/hexane. The pure fractions were combined and concentrated under reduced pressure to give ethyl 6-(4-(3-(4-cyano-3-(trifluoromethyl)phenyl)-5,5-dimethyl-4-oxo-2-thioxoimidazolidin-1-yl)-2-fluorobenzamido)hexanoate (Int-5, 2.39 g, 70%) as an off-white solid.

¹H NMR (400 MHz, DMSO-d₆) δ 8.48 (t, J=5.38 Hz, 1H), 8.39 (d, J=8.31 Hz, 1H), 8.28 (d, J=1.22 Hz, 1H), 8.07 (dd, J=8.31, 1.47 Hz, 1H), 7.74 (t, J=8.07 Hz, 1H), 7.41 (dd, J=10.64, 1.59 Hz, 1H), 7.31 (dd, J=8.07, 1.71 Hz, 1H), 4.04 (q, J=7.09 Hz, 2H), 3.24 (q, J=6.60 Hz, 2H), 2.28 (t, J=7.34 Hz, 2H), 1.55-1.59 (m, 2H), 1.53 (s, 6H), 1.47-1.52 (m, 2H), 1.28-1.38 (m, 2H), 1.16 (t, J=7.09 Hz, 3H). LCMS: 593.2 [M+H]⁺.

### Step-6: Preparation of 6-(4-(3-(4-Cyano-3-(trifluoromethyl)phenyl)-5,5-dimethyl-4-oxo-2-thioxoimidazolidin-1-yl)-2-fluorobenzamido)hexanoic Acid (Int-6)

To a flask charged with ethyl 6-(4-(3-(4-cyano-3-(trifluoromethyl)phenyl)-5,5-dimethyl-4-oxo-2-thioxoimidazolidin-1-yl)-2-fluorobenzamido)hexanoate (Int-5, 2.4 g, 4.05 mmol, 1.0 eq.) in EtOH:THF:H₂O (1:1:1, 24 mL, 10 vol), LiOH (509 mg, 12.15 mmol, 3.0 eq.) was added at ambient temperature under argon atmosphere. The reaction mixture was allowed to stir at ambient temperature until TLC indicated complete consumption of the starting material. The reaction mixture was concentrated under reduced pressure, diluted with water (10 mL), acidified with citric acid to pH˜3, filtered the obtained solid and dried to obtain 6-(4-(3-(4-cyano-3-(trifluoromethyl)phenyl)-5,5-dimethyl-4-oxo-2-thioxoimidazolidin-1-yl)-2-fluorobenzamido)hexanoic acid (Int-6, 2.0 g, 87%) as an off-white solid.

¹H NMR (400 MHz, DMSO-d₆) δ 11.99 (s, 1H), 8.49 (t, J=5.38 Hz, 1H), 8.40 (d, J=8.31 Hz, 1H), 8.29 (s, 1H), 8.08 (d, J=8.31 Hz, 1H), 7.75 (t, J=8.07 Hz, 1H), 7.42 (d, J=10.27 Hz, 1H), 7.33 (dd, J=8.07, 1.22 Hz, 1H), 3.26 (q, J=6.36 Hz, 2H), 2.22 (t, J=7.34 Hz, 2H), 1.47-1.59 (m, 10H), 1.29-1.39 (m, 2H). LCMS: 565.10 [M+H]⁺.

### Step-7: Preparation of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-(4-(3-(4-cyano-3-(trifluoro methyl)phenyl)-5,5-dimethyl-4-oxo-2-thioxoimidazolidin-1-yl)-2-fluorobenzamido)hexanoyl) piperazine-1-carboxylate

A flask was charged with 6-(4-(3-(4-cyano-3-(trifluoromethyl)phenyl)-5,5-dimethyl-4-oxo-2-thioxoimidazolidin-1-yl)-2-fluorobenzamido)hexanoic acid (Int-6, 1.0 g, 1.77 mmol, 1.0 eq.) in DMF (10 mL), (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate (Int-A. 945 mg, 1.77 mmol, 1.0 eq.), HATU (1.0 g, 2.65 mmol, 1.5 eq.) and DIPEA (0.97 mL, 3.98 mmol, 3.0 eq.) were added at ambient temperature under argon atmosphere. The reaction mixture was allowed to stir at ambient temperature until TLC indicated complete consumption of starting material, diluted with ice cold water (100 mL) and extracted ethyl acetate (2×200 mL). The combined organic layer was washed with brine solution (200 mL), dried over sodium sulfate, filtered, concentrated under reduced pressure and purified by flash column chromatography (silica gel, 100-200 mesh) eluting with 0-5% MeOH in DCM. The pure fractions were combined and concentrated under reduced pressure to obtained (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-(4-(3-(4-cyano-3-(trifluoro methyl)phenyl)-5,5-dimethyl-4-oxo-2-thioxoimidazolidin-1-yl)-2-fluoro benzamide)hexanoyl)piperazine-1-carboxylate (605 mg, 31%) as an off-white solid.

¹H NMR (400 MHz, DMSO-d₆) δ 8.95 (s, 1H), 8.47-8.54 (m, 1H), 8.40 (d, J=8.38 Hz, 1H), 8.29 (s, 1H), 8.04-8.16 (m, 2H), 7.76 (t, J=7.63 Hz, 1H), 7.66 (d, J=8.63 Hz, 1H), 7.43 (d, J=10.76 Hz, 1H), 7.30-7.38 (m, 2H), 6.51 (s, 1H), 5.40-5.46 (m, 2H), 5.28-5.35 (m, 2H), 3.41-3.85 (m, 10H), 2.35-2.44 (m, 2H), 2.20 (s, 6H), 1.80-1.95 (m, 2H), 1.50-1.64 (m, 10H), 1.33-1.44 (m, 2H), 1.20-1.30 (m, 2H), 0.89 (t, J=6.69 Hz, 3H). LCMS: 1080.30 [M+H]⁺. HPLC purity 96.1%.

### Example S10. Preparation of (S)-10-((4-(1-(6-(((1r,4r)-4-(3-Chloro-4-cyanophenoxy)cyclohexyl)carbamoyl)pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl 4-Methylpiperazine-1-carboxylate (Compound No. 10)

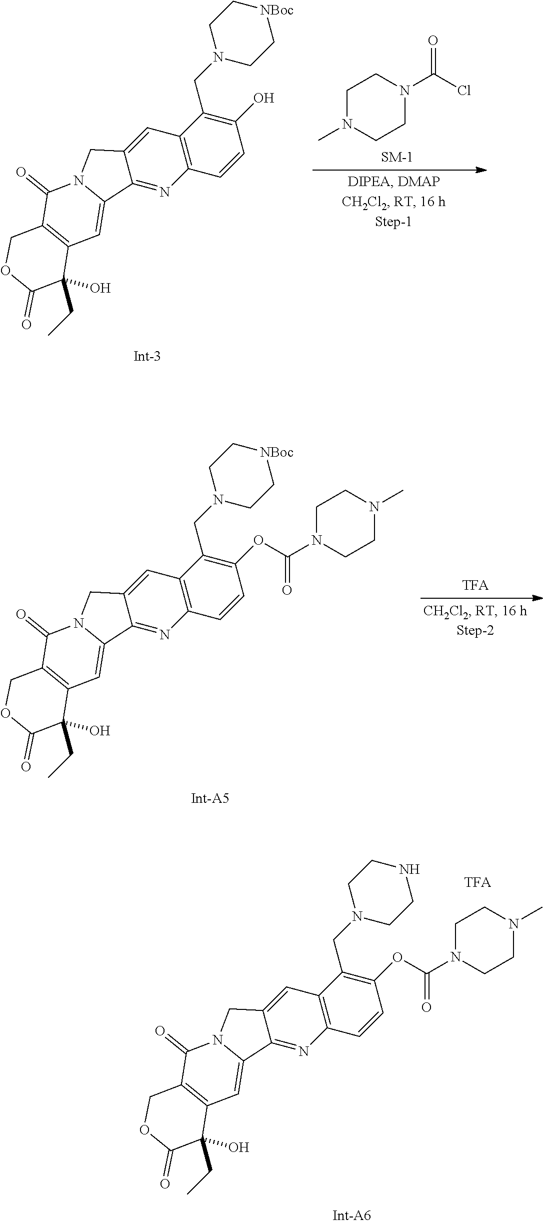

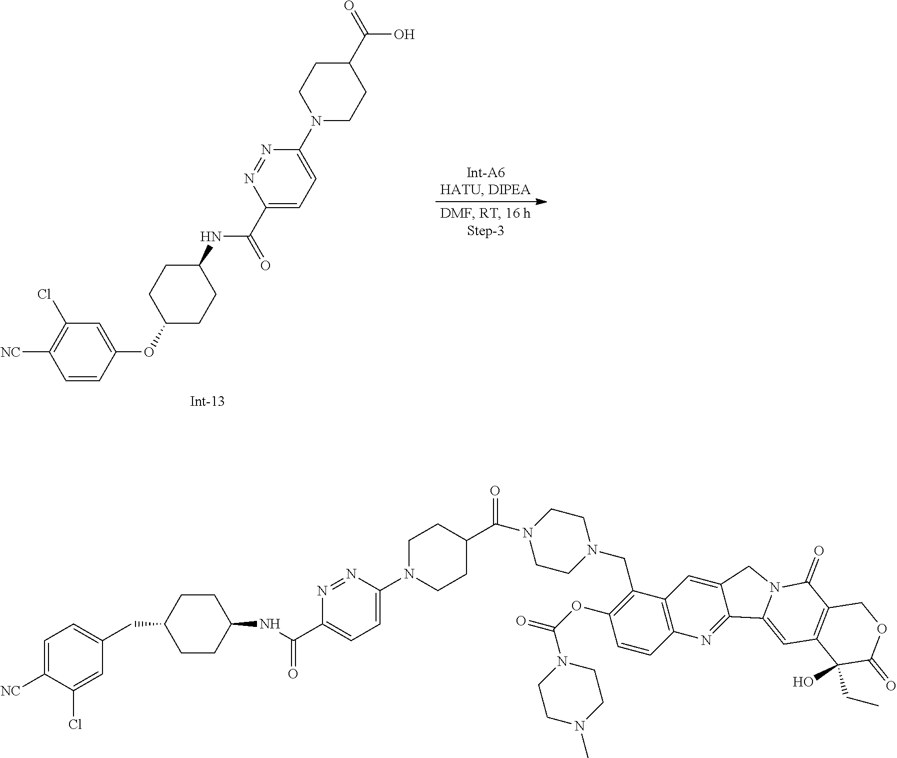 [SMILES: N#Cc1ccc(O[C@H]2CC[C@H](NC(=O)c3ccc(N4CCC(C(=O)O)CC4)nn3)CC2)cc1Cl ; CC[C@@]1(O)C(=O)OCc2c1cc1n(c2=O)Cc2cc3c(CN4CCN(C(=O)C5CCN(c6ccc(C(=O)N[C@H]7CC[C@H](Cc8ccc(C#N)c(Cl)c8)CC7)nn6)CC5)CC4)c(OC(=O)N4CCN(C)CC4)ccc3nc2-1]

### Step-1: Preparation of (S)-10-((4-(tert-Butoxycarbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl 4-Methylpiperazine-1-carboxylate (Int-A5)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-3, 300 mg, 0.53 mmol, 1.0 eq.) in DCM (15 mL) were added DIPEA (0.3 mL, 1.60 mmol, 3 eq.) and DMAP (20 mg, 0.16 mmol, 0.25 eq.) followed by addition of 4-methylpiperazine-1-carbonyl chloride (SM-1, 129 mg, 0.80 mmol, 1.5 eq.) in DCM (5 mL) at room temperature and stirred for 16 h. Progress of the reaction was monitored by TLC. After completion of the reaction, the reaction mixture was washed with water (100 mL) and extracted with DCM (2×200 mL). The combined organic extract was washed with brine (100 mL) and dried over anhydrous sodium sulfate, filtered and concentrated under vacuum to obtain the crude product. The crude obtained was purified by combiflash column eluting with 6% methanol in DCM to afford Int-A5 (260 mg, 70%) as an off-white solid.

¹H NMR (400 MHz, DMSO-d₆) δ 9.01 (s, 1H), 8.12 (d, J=9.29 Hz, 1H), 7.63 (d, J=8.80 Hz, 1H), 7.34 (s, 1H), 6.51 (s, 1H), 5.43 (s, 2H), 5.30 (s, 2H), 3.87 (s, 2H), 3.68-3.70 (m, 2H), 3.48-3.50 (m, 2H), 3.20-3.25 (m, 2H), 2.38-2.42 (m, 8H), 2.25 (s, 3H), 1.84-1.91 (m, 2H), 1.39 (s, 9H), 1.25 (br dd, J=11.98, 6.11 Hz, 2H), 0.89 (br t, J=7.09 Hz, 3H). LCMS: 689.4 [M+H]⁺.

### Step-2: Preparation of (S)-4-Ethyl-4-hydroxy-3,14-dioxo-10-(piperazin-1-ylmethyl)-3,4,12,14-tetrahydro-1H-pyrano[3′,4′:6,7]indolizino[1,2-b]quinolin-9-yl 4-Methylpiperazine-1-carboxylate Trifluoroacetate Salt (Int-A6)

To a stirred solution of (S)-10-((4-(tert-butoxycarbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl 4-methylpiperazine-1-carboxylate (Int-A6, 250 mg, 0.36 mmol, 1.0 eq.) in DCM (10 mL) under nitrogen atmosphere was added TFA (0.3 mL, 3.5 mmol, 10 eq.) at 0° C. The reaction mixture was allowed to warm up to room temperature and stir for 16 h. After completion of the reaction, solvents were evaporated under reduced pressure, washed with diethyl ether (20 mL) and dried under vacuum to afford Int-A6 (210 mg, 98%) as an off-white solid.

¹H NMR (400 MHz, DMSO-d₆) δ 9.04 (s, 1H), 8.66 (br s, 2H), 8.18 (d, J=9.29 Hz, 1H), 7.69 (d, J=9.29 Hz, 1H), 7.35 (s, 1H), 6.55 (br s, 1H), 5.44 (s, 2H), 5.31 (s, 2H), 4.40 (br s, 1H), 4.16 (br s, 1H), 3.96-3.99 (m, 3H), 3.49-3.54 (m, 3H), 3.20-3.22 (m, 2H), 3.01-3.05 (m, 4H), 2.90 (s, 3H), 2.62-2.68 (m, 4H), 1.82-1.93 (m, 2H), 0.89 (br t, J=7.09 Hz, 3H). LCMS: 589.2 [M+H]⁺.

### Step-3: Preparation of (S)-10-((4-(1-(6-(((1r,4r)-4-(3-chloro-4-cyanophenoxy)cyclohexyl) carbamoyl)pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl 4-Methylpiperazine-1-carboxylate

To a stirred solution of (S)-4-ethyl-4-hydroxy-3,14-dioxo-10-(piperazin-1-ylmethyl)-3,4,12,14-tetrahydro-1H-pyrano[3′,4′:6,7]indolizino[1,2-b]quinolin-9-yl 4-methylpiperazine-1-carboxylate TFA salt (Int-A6, 200 mg, 0.34 mmol, 1.0 eq.) and 1-(6-(((1r,4r)-4-(3-chloro-4-cyanophenoxy)cyclohexyl)carbamoyl)pyridazin-3-yl)piperidine-4-carboxylic acid (Int-13, 185 mg, 0.37 mmol, 1.1 eq.) in DMF (5 mL) were added HATU (243 mg, 0.68 mmol, 2 eq.) and DIPEA (0.177 mL, 1.02 mmol, 3 eq.) at room temperature and stirred for 16 h. Progress of the reaction was monitored by TLC. After completion of the reaction, the reaction mixture was washed with water (60 mL) and extracted with ethyl acetate (2×100 mL). The combined organic extract was washed with brine (100 mL) and dried over anhydrous sodium sulfate, filtered and concentrated under vacuum to obtain the crude product. The crude obtained was purified by combiflash column eluting with 5% methanol in DCM to afford the title compound (86 mg, 24%) as an off-white solid.

¹H NMR (400 MHz, DMSO-d₆) δ 9.05 (s, 1H), 8.57 (br d, J=8.25 Hz, 1H), 8.14 (d, J=9.26 Hz, 1H), 7.83 (dd, J=15.76, 9.13 Hz, 2H), 7.65 (d, J=9.26 Hz, 1H), 7.30-7.45 (m, 3H), 7.13 (dd, J=8.82, 2.44 Hz, 1H), 6.52 (s, 1H), 5.44 (s, 2H), 5.32 (s, 2H), 4.40-4.58 (m, 3H), 3.84-3.94 (m, 3H), 3.69-3.71 (m, 2H), 3.37-3.56 (m, 6H), 3.11 (br t, J=11.94 Hz, 3H), 2.38-2.42 (m, 6H), 2.26 (s, 3H), 2.05-2.14 (m, 2H), 1.84-1.95 (m, 4H), 1.46-1.76 (m, 10H), 0.89 (t, J=7.32 Hz, 3H). LCMS: 1055.65 [M+H]⁺. HPLC purity 96.0%.

### Example S11. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(1-(6-(((1r,4r)-4-((3-chloro-4-cyanophenyl)(methyl)amino)-cyclohexyl)carbamoyl)pyridazin-3-yl)piperidine-4-carbonyl)piperazine-1-carboxylate (Compound No. 11)

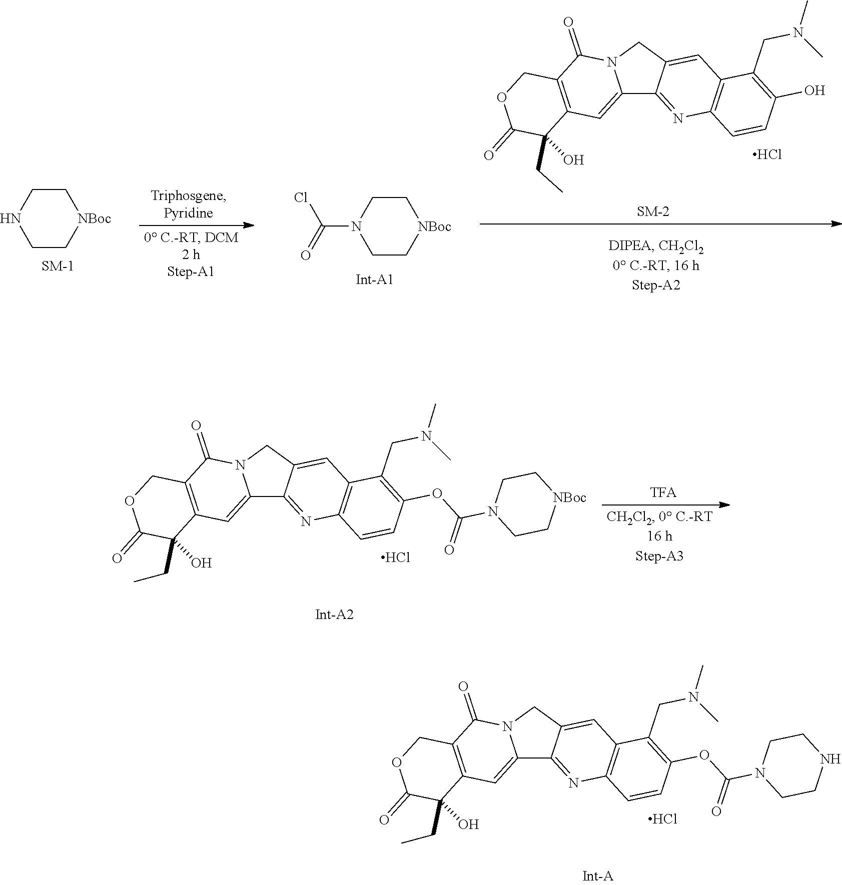

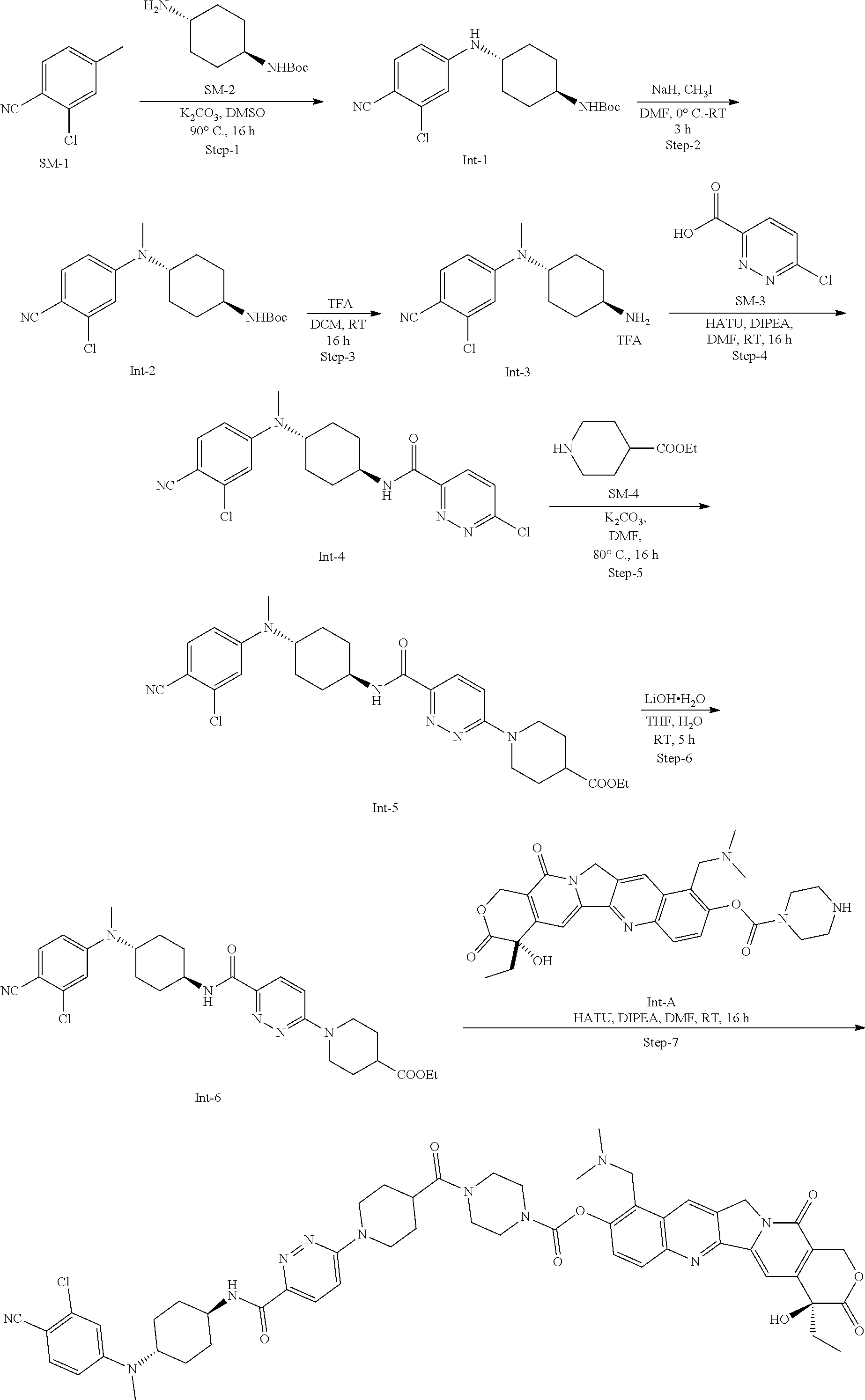

### Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate (Int-A)

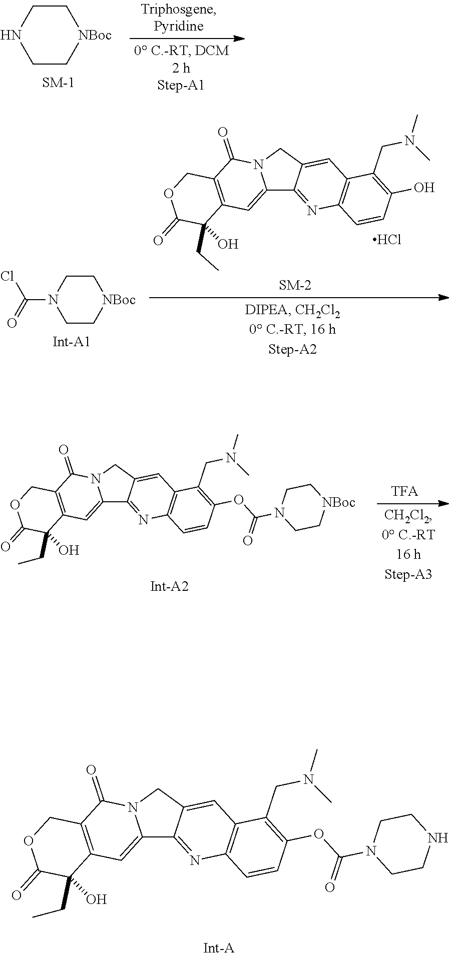 [SMILES: CC(C)(C)OC(=O)N1CCNCC1 ; CC[C@@]1(O)C(=O)OCc2c1cc1n(c2=O)Cc2cc3c(CN(C)C)c(O)ccc3nc2-1 ; Cl ; CC(C)(C)OC(=O)N1CCN(C(=O)Cl)CC1 ; CC[C@@]1(O)C(=O)OCc2c1cc1n(c2=O)Cc2cc3c(CN(C)C)c(OC(=O)N4CCN(C(=O)OC(C)(C)C)CC4)ccc3nc2-1 ; CC[C@@]1(O)C(=O)OCc2c1cc1n(c2=O)Cc2cc3c(CN(C)C)c(OC(=O)N4CCNCC4)ccc3nc2-1]

### Step-A1: Preparation of tert-Butyl 4-(Chlorocarbonyl)piperazine-1-carboxylate (Int-A1)

To a stirred solution of tert-butyl piperazine-1-carboxylate (SM-1, 5 g, 26.8 mmol, 1.0 eq.) in DCM (100 mL) were added pyridine (2.97 g, 37.6 mmol, 1.4 eq.) and triphosgene (3.19 g, 10.7 mmol, 0.4 eq.) at 0° C. The reaction mixture was allowed to warm up to room temperature and stir for 2 h. Progress of the reaction was monitored by TLC. After completion of the reaction, the reaction mixture was washed with water (100 mL) and extracted with DCM (2×100 mL). The combined organic extract was again washed with water (200 mL), brine (200 mL) and dried over anhydrous sodium sulfate, filtered, and concentrated under vacuum to afford Int-A1 (6.0 g, 90%) as a crude oil.

¹H NMR (400 MHz, DMSO-d₆) δ 3.64 (d, J=4.40 Hz, 2H), 3.52 (br s, 2H), 3.27-3.47 (m, 3H), 2.93-3.21 (m, 1H), 1.41 (s, 9H).

### Step-A2: Preparation of (S)-1-(tert-Butyl) 4-(10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) Piperazine-1,4-dicarboxylate (Int-A2)

To a stirred solution of (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione HCl salt (SM-2, 10 g, 23.7 mmol, 1.0 eq.) in DCM (250 mL) were added DIPEA (15.3 g, 118 mmol, 5 eq.) and DMAP (724 mg, 5.9 mmol, 0.25 eq.) followed by addition of tert-butyl 4-(chlorocarbonyl)piperazine-1-carboxylate (Int-A1, 5.89 g, 23.7 mmol, 1 eq.) in DCM (100 mL) dropwise over a period of 10 min at 0° C. The resulting reaction mixture was allowed to war up to room temperature and allowed to stir for 16 h. Progress of the reaction was monitored by TLC. After reaction completion, the mixture was washed with water (100 mL) and extracted with DCM (3×100 mL). The combined organic extract was again washed with water (200 mL), brine (200 mL) and dried over anhydrous sodium sulfate, filtered and concentrated under vacuum to get the crude product. The crude obtained was purified by combiflash column using 7% methanol in DCM to afford Int-A2 (10 g, 66%) as an off-white solid.

¹H NMR (400 MHz, DMSO-d₆) δ 8.95 (s, 1H), 8.11 (d, J=9.29 Hz, 1H), 7.65 (d, J=9.29 Hz, 1H), 7.34 (s, 1H), 6.53 (s, 1H), 5.43 (s, 2H), 5.31 (s, 2H), 3.67-3.79 (m, 4H), 3.41-3.55 (m, 6H), 2.20 (s, 6H), 1.80-1.93 (m, 2H), 1.44 (s, 9H), 0.89 (t, J=7.34 Hz, 3H). LCMS: 634.2 [M+H]⁺.

### Step-3: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl Piperazine-1-carboxylate (Int-A)

To a stirred solution of (S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) piperazine-1,4-dicarboxylate (Int-A2, 1 g, 15 mmol, 1.0 eq.) in DCM (20 mL) under nitrogen atmosphere was added TFA (3 mL) at 0° C. The reaction mixture was warmed up to room temperature and allowed to stir for 16 h. Progress of the reaction was monitored by TLC. After completion of the reaction, solvents were evaporated under reduced pressure, saturated NaHCO₃ solution (50 mL) added and the aqueous solution was extracted with EtOAc (2×100 mL). Solvent was evaporated under reduced pressure to afford Int-A (1.02 g, crude) as off-white solid.

¹H NMR (400 MHz, DMSO-d₆) δ 10.12 (br s, 1H), 9.03-9.27 (m, 2H), 8.35 (d, J=9.29 Hz, 1H), 7.86 (d, J=9.29 Hz, 1H), 7.37 (s, 1H), 6.57 (br s, 1H), 5.45 (br s, 2H), 5.33 (br s, 2H), 4.86 (br s, 2H), 3.90-3.95 (m, 2H), 3.68-3.71 (m, 2H), 3.30 (d, J=12.23 Hz, 2H), 2.89 (s, 6H), 1.80-1.95 (m, 3H), 1.08 (t, J=7.09 Hz, 1H), 0.89 (br t, J=6.85 Hz, 3H). LCMS: 534.2 [M+H]⁺.

### Step-1: Preparation of tert-Butyl ((1r,4r)-4-((3-Chloro-4-cyanophenyl)amino)cyclohexyl)carbamate (Int-1)

To a stirred solution of 2-chloro-4-fluorobenzonitrile (SM-1, 4 g, 25 mmol, 1.0 eq.) in DMSO (40 mL) were added tert-butyl ((1r,4r)-4-aminocyclohexyl)carbamate (SM-2, 5.5 g, 25 mmol, 1.0 eq.) and K₂CO₃ (7.1 g, 51 mmol, 2 eq.) at room temperature. The reaction mixture was heated to 90° C. for 16 h. Progress of the reaction was monitored by TLC. After completion of the reaction, the reaction mixture was diluted with ice cold water (200 mL) and extracted with ethyl acetate (2×400 mL). The combined organic extract was washed with water (200 mL), brine (200 mL), dried over anhydrous sodium sulfate, filtered and concentrated under vacuum to obtain the crude product. The crude obtain was purified by combiflash column eluting with 64% ethyl acetate in heptane to afford Int-1 (7.1 g, 78%) as an off-white solid.

¹H NMR (400 MHz, DMSO-d₆) δ 7.48 (d, J=8.80 Hz, 1H), 6.86 (d, J=6.85 Hz, 1H), 6.80 (d, J=6.85 Hz, 1H), 6.75 (br s, 1H), 6.59 (d, J=8.80 Hz, 1H), 3.20-3.23 (m, 2H), 1.91 (d, J=11.25 Hz, 2H), 1.79 (d, J=10.76 Hz, 2H), 1.38 (s, 9H), 1.13-1.33 (m, 4H).

### Step-2: Preparation of tert-Butyl ((1r,4r)-4-((3-Chloro-4-cyanophenyl)(methyl)amino)cyclohexyl)carbamate (Int-2)

To a stirred solution of tert-butyl ((1r,4r)-4-((3-chloro-4-cyanophenyl)amino)cyclohexyl)carbamate (Int-1, 5.5 g, 15 mmol, 1.0 eq.) in DMF (25 mL) under nitrogen atmosphere was added NaH (63%, 500 mg, 21 mmol, 1.3 eq.) portionwise at 0° C. The reaction mixture was allowed to warm up to room temperature, stir for 30 min, and then methyl iodide (1.1 mL, 21 mmol, 1.3 eq.) was added dropwise at 0° C. Reaction mixture was allowed to warm up to room temperature and stir for 3 h. Progress of the reaction was monitored by TLC. After completion of the reaction, water (150 mL) was added and extracted with ethyl acetate (2×200 mL). Solvents were evaporated under reduced pressure to get the crude product which was purified by combiflash column eluting with 15% ethyl acetate in heptane in to afford Int-2 (3.2 g, 55%) as an off-white solid.

LCMS: 364.2 [M+H]⁺.

### Step-3: Preparation of 4-(((1r,4r)-4-Aminocyclohexyl)(methyl)amino)-2-chlorobenzonitrile Trifluoroacetate Salt (Int-3)

To a stirred solution of tert-butyl ((1r,4r)-4-((3-chloro-4-cyanophenyl)(methyl)amino)cyclohexyl)-carbamate Int-2 (1.1 g, 3 mmol, 1.0 eq.) in DCM (10 mL) under nitrogen atmosphere was added TFA (10 mL, 10 vol) at 0° C. The reaction mixture was allowed to war up to room temperature and stir for 16 h. Progress of the reaction was monitored by TLC. After completion of the reaction, solvents were evaporated under reduced pressure, washed with diethyl ether (20 mL) and dried under vacuum to afford Int-3 (852 mg, 74%) as an off-white solid.

LCMS: 264.1 [M+H]⁺.

### Step-4: Preparation of 6-Chloro-N-((1r,4r)-4-((3-chloro-4-cyanophenyl)(methyl)amino)cyclohexyl)pyridazine-3-carboxamide (Int-4)

To a stirred solution of 4-(((1r,4r)-4-aminocyclohexyl)(methyl)amino)-2-chlorobenzonitrile trifluoroacetate salt (Int-3, 800 mg, 8 mmol, 1.0 eq.) and 6-chloropyridazine-3-carboxylic acid (SM-3, 336 mg, 8 mmol, 1.0 eq.) in DMF (3 mL) were added HATU (1.21 g, 12 mmol, 1.5 eq.) and DIPEA (0.74 mL, 16 mmol, 2 eq.) at room temperature and the resulting reaction mixture was allowed to stir for 16 h. Progress of the reaction was monitored by TLC. After completion of the reaction, water (50 mL) was added and extracted with ethyl acetate (2×100 mL). The combined organic extract was washed with water (100 mL), brine (100 mL) and dried over anhydrous sodium sulfate. Solvents were evaporated under reduced pressure to obtain the crude product, which was purified by combiflash column eluting with 4% methanol in DCM to afford Int-4 (700 mg, 81%) as an off-white solid.

¹H NMR (400 MHz, DMSO-d₆) δ 8.44 (d, J=9.25 Hz, 1H), 8.21 (d, J=9.25 Hz, 1H), 7.95 (s. 1H), 7.68 (dd, J=8.32, 4.62 Hz, 1H), 7.58 (d, J=9.25 Hz, 1H), 6.80 (dd, J=9.25, 2.31 Hz, 1H), 3.79-3.89 (m, 1H), 3.70-3.73 (m, 1H), 2.69 (s, 3H), 1.84-1.87 (m, 2H), 1.51-1.77 (m, 6H).

LCMS: 404.1 [M+H]⁺.

### Step-5: Preparation of Ethyl 1-(6-(((1r,4r)-4-((3-chloro-4-cyanophenyl)(methyl)amino)cyclohexyl)carbamoyl)-pyridazin-3-yl)piperidine-4-carboxylate (Int-5)

To a stirred solution of 6-chloro-N-((1r,4r)-4-((3-chloro-4-cyanophenyl)(methyl)amino)cyclohexyl)-pyridazine-3-carboxamide (Int-4, 250 mg, 0.6 mmol, 1.0 eq.) and ethyl piperidine-4-carboxylate (SM-4, 0.1 mL, 0.6 mmol, 1.0 eq.) in DMF (2 mL) was added K₂CO₃ (129 mg, 0.9 mmol, 1.5 eq.) at room temperature. The reaction mixture was heated to 80° C. for 16 h. Progress of the reaction was monitored by TLC. After reaction completion, water (50 mL) was added and the aqueous mixture was extracted with ethyl acetate (2×100 mL). The combined organic extract was washed with water (100 mL), brine (100 mL) and dried over anhydrous sodium sulfate. Solvents were evaporated under reduced pressure to give the crude product which was purified by combiflash column eluting with 62% ethyl acetate in heptane to afford Int-5 (198 mg, 60%) as a pale yellow solid.

¹H NMR (400 MHz, DMSO-d₆) δ 7.95 (s, 1H), 7.82 (d, J=9.78 Hz, 1H), 7.60 (d, J=9.29 Hz, 1H), 7.36 (d, J=9.29 Hz, 1H), 6.94 (d, J=2.45 Hz, 1H), 6.83 (dd, J=9.29, 2.45 Hz, 1H), 4.35-4.37 (m, 2H), 4.08 (q, J=7.34 Hz, 2H), 3.76-3.88 (m, 2H), 3.12-3.22 (m, 2H), 2.85 (s, 3H), 2.69-2.76 (m, 4H), 1.89-1.95 (m, 3H), 1.64-1.76 (m, 4H), 1.54-1.60 (m, 2H), 1.19 (t, J=7.09 Hz, 3H). LCMS: 525.2 [M+H]⁺.

### Step-6: Preparation of 1-(6-(((1r,4r)-4-((3-Chloro-4-cyanophenyl)(methyl)amino)cyclohexyl)carbamoyl)-pyridazin-3-yl)piperidine-4-carboxylic acid (Int-6)

To a stirred solution of ethyl 1-(6-(((1r,4r)-4-((3-chloro-4-cyanophenyl)(methyl)amino)cyclohexyl)-carbamoyl)pyridazin-3-yl)piperidine-4-carboxylate (Int-5, 190 mg, 0.36 mmol, 1.0 eq.) in THF (2 mL) and water (1 mL) was added LiOH (27 mg, 1.1 mmol, 3 eq.) at room temperature and stirring was continued for 5 h. Progress of the reaction was monitored by TLC. After completion of the reaction, solvents were evaporated under reduced pressure and water (10 mL) was added and the reaction mixture was acidified with 1M HCl (6 mL) pH 6. Filtered the precipitate and dried under vacuum to afford Int-6 (158 mg, 87%) as pale yellow solid.

¹H NMR (400 MHz, DMSO-d₆) δ 12.26 (br s, 1H), 8.50 (d, J=8.31 Hz, 1H), 7.82 (d, J=9.29 Hz, 1H), 7.60 (d, J=8.80 Hz, 1H), 7.36 (d, J=9.78 Hz, 1H), 6.94 (br s, 1H), 6.83 (d, J=8.80 Hz, 1H), 4.34-4.36 (m, 2H), 3.73-3.93 (m, 1H), 3.16 (t, J=11.74 Hz, 2H), 2.85 (s, 3H), 2.61 (t, J=10.27 Hz, 1H), 1.92 (d, J=10.76 Hz, 4H), 1.48-1.80 (m, 8H), 1.14-1.28 (m, 1H). LCMS: 497.2 [M+H]⁺.

### Step-7: Preparation of 6 (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(1-(6-(((1r,4r)-4-((3-chloro-4-cyanophenyl)(methyl)-amino)cyclohexyl)carbamoyl)pyridazin-3-yl)piperidine-4-carbonyl)piperazine-1-carboxylate

To a stirred solution of 1-(6-(((1r,4r)-4-((3-chloro-4-cyanophenyl)(methyl)amino)cyclohexyl)-carbamoyl)pyridazin-3-yl)piperidine-4-carboxylic acid (Int-6, 110 mg, 0.22 mmol, 1.0 eq.) and (S)-10-((dimethyl-amino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate (Int-A, 118 mg, 0.22 mmol, 1.0 eq.) in DMF (3 mL) were added HATU (126 mg, 0.33 mmol, 1.5 eq.) and DIPEA (0.12 mL, 0.66 mmol, 3 eq.) at room temperature and stirring was continued for 16 h. Progress of the reaction was monitored by TLC. After completion of the reaction, water (25 mL) was added and aqueous mixture extracted with ethyl acetate (2×30 mL), The combined organic extract was washed with water (50 mL), brine (50 mL) and dried over anhydrous sodium sulfate. Solvents were evaporated under reduced pressure to obtain the crude product which was purified by combiflash column chromatography eluting with 10% methanol in DCM to afford the title compound (65 mg, 19%) as an off-white solid.

¹H NMR (400 MHz, DMSO-d₆) δ 8.96 (s, 1H), 8.48 (d, J=7.82 Hz, 1H), 8.13 (d, J=9.29 Hz, 1H), 7.84 (d, J=9.78 Hz, 1H), 7.67 (d, J=8.80 Hz, 1H), 7.60 (d, J=9.29 Hz, 1H), 7.38 (d, J=9.29 Hz, 1H), 7.34 (s, 1H), 6.93-6.96 (m, 1H), 6.80-6.86 (m, 1H), 6.51 (s, 1H), 5.43 (s, 2H), 5.32 (s, 2H), 4.47-4.57 (m, 2H), 3.82-3.90 (m, 1H), 3.78 (s, 3H), 3.43-3.72 (m, 6H), 3.04-3.21 (m, 4H), 2.85 (s, 3H), 2.22 (s, 6H), 1.85-1.97 (m, 5H), 1.72-1.84 (m, 3H), 1.54-1.71 (m, 7H), 0.89 (t, J=7.09 Hz, 3H). LCMS: 1010.60 [M−H]⁻. HPLC purity 95.8%.

### Example S12. Preparation of (S)-10-((Dimethylamino)methyl)-4-ethyl-4-(heptanoyloxy)-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Compound No. 12)

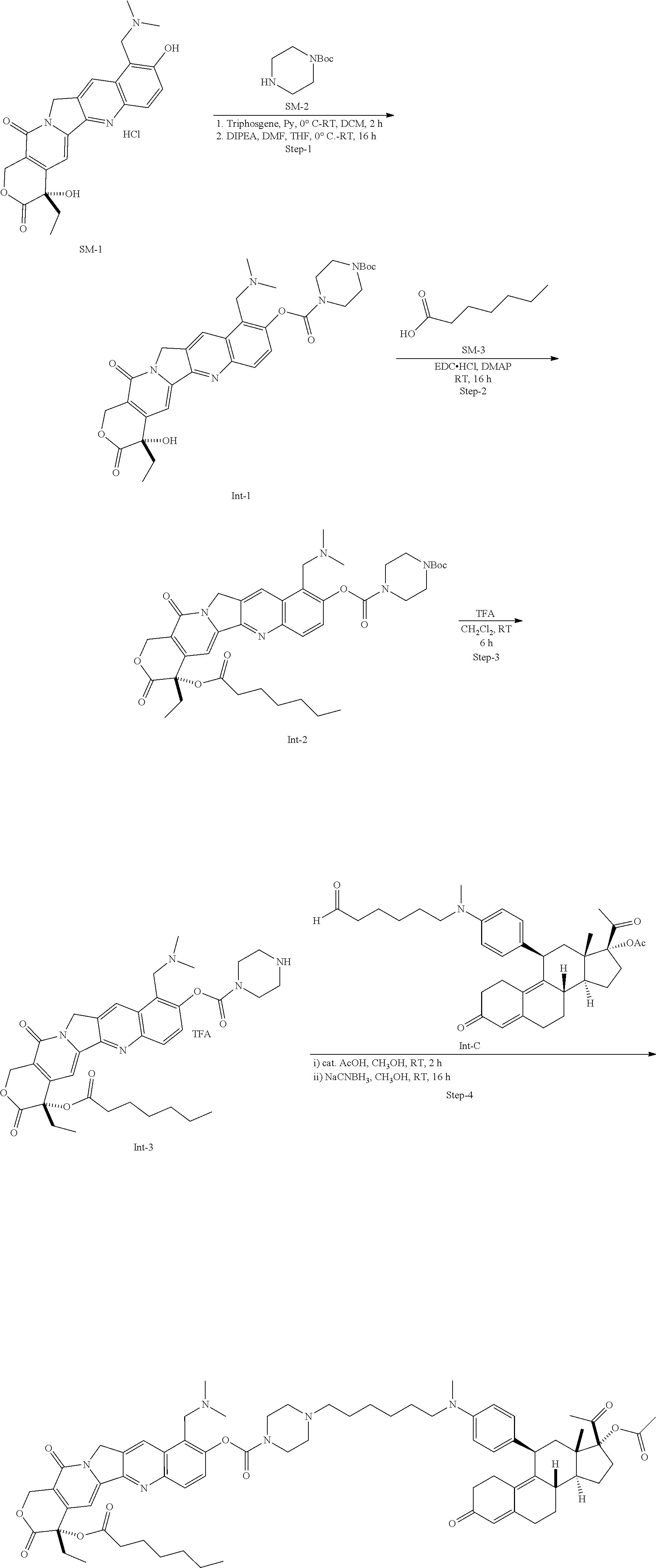

### Step-1-1: Preparation of tert-Butyl 4-(Chlorocarbonyl)piperazine-1-carboxylate

To a stirred solution of tert-butyl piperazine-1-carboxylate (SM-2, 5 g, 26.8 mmol, 1.0 eq.) in DCM (100 mL) were added pyridine (2.97 g, 37.6 mmol, 1.4 eq.) and triphosgene (3.19 g, 10.7 mmol, 0.4 eq.) at 0° C. The reaction mixture was allowed to warm up to room temperature and stir for 2 h. Progress of the reaction was monitored by TLC. After completion of starting material, the reaction mixture was washed with water (100 mL) and extracted with DCM (2×100 mL). The combined organic extract was again washed with water (200 mL), brine (200 mL) and dried over anhydrous sodium sulfate, filtered, and concentrated under vacuum to afford tert-butyl 4-(chlorocarbonyl)piperazine-1-carboxylate (6.0 g, 90%) as a crude oil.

¹H NMR (400 MHz, DMSO-d₆) δ 3.64 (br d, J=4.40 Hz, 2H), 3.52 (br s, 2H), 3.27-3.47 (m, 3H), 2.93-3.21 (m, 1H), 1.41 (s, 9H).

### Step-1-2: Preparation of (S)-1-(tert-Butyl) 4-(10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)piperazine-1,4-dicarboxylate (Int-1)

To a stirred solution of (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione hydrochloride (SM-1, 10 g, 23.7 mmol, 1.0 eq.) in DCM (100 mL, 10 vol) were added DIPEA (15.3 g, 118 mmol, 5 eq.) and DMAP (724 mg, 5.9 mmol, 0.25 eq.) followed by addition of tert-butyl 4-(chlorocarbonyl)piperazine-1-carboxylate (5.89 g, 23.7 mmol, 1 eq.) in DCM (100 mL, 10 vol) dropwise over a period of 10 min at 0° C. The resulting reaction mixture was allowed to warm up to room temperature and stir for 16 h. Progress of the reaction was monitored by TLC. After completion of the reaction, the mixture was washed with water (500 mL), extracted with DCM (3×100 mL) and the combined organic extract was again washed with water (200 mL), brine (200 mL) and dried over anhydrous sodium sulfate, filtered and concentrated under vacuum. The crude obtained was purified by combiflash column eluting with 7% methanol in DCM to afford Int-1 (10 g, 66%) as an off-white solid.

¹H NMR (400 MHz, DMSO-d₆) δ 8.95 (s, 1H), 8.11 (d, J=9.29 Hz, 1H), 7.65 (d, J=9.29 Hz, 1H), 7.34 (s, 1H), 6.53 (s, 1H), 5.43 (s, 2H), 5.31 (s, 2H), 3.67-3.79 (m, 4H), 3.41-3.55 (m, 6H), 2.20 (s, 6H), 1.80-1.93 (m, 2H), 1.44 (s, 9H), 0.89 (br t, J=7.34 Hz, 3H). LCMS: 634.2 [M+H]⁺.

### Step-2: Preparation of (S)-1-(tert-Butyl) 4-(10-((Dimethylamino)methyl)-4-ethyl-4-(heptanoyloxy)-3,14-dioxo-3,4,12,14-tetrahydro-1H-pyrano[3′,4′:6,7]indolizino[1,2-b]quinolin-9-yl) Piperazine-1,4-dicarboxylate (Int-2)

To a stirred solution of (S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) piperazine-1,4-dicarboxylate (Int-2, 7.0 g, 11.05 mmol, 1.0 eq.) in DCM (70 mL, 10.0 vol), heptanoic acid (SM-3, 2.15 g, 16.57 mmol, 1.5 eq.), EDC·HCl (3.168 g, 16.57 mmol, 1.5 eq.) and DMAP (606 mg, 4.97 mmol, 0.45 eq.) were added at 0° C. under argon atmosphere. The reaction mixture was allowed to stir at RT until TLC indicated complete consumption of starting material. The reaction mixture was then diluted with ice cold water (250 mL), extracted with DCM (2×250 mL), combined organic layer washed with brine solution (200 mL), dried over anhydrous sodium sulfate, filtered and concentrated under reduced pressure. The crude obtained was purified by flash column and the pure fractions were concentrated under reduced pressure to obtain (S)-1-(tert-butyl) 4-(10-((dimethylamino)methyl)-4-ethyl-4-(heptanoyloxy)-3,14-dioxo-3,4,12,14-tetrahydro-1H-pyrano[3′,4′:6,7]indolizino[1,2-b]quinolin-9-yl)piperazine-1,4-dicarboxylate (Int-2, 6 g, 73%) as an off-white solid.

¹H NMR (400 MHz, DMSO-d₆) δ 8.95 (s, 1H), 8.07 (d, J=8.80 Hz, 1H), 7.65 (d, J=9.29 Hz, 1H), 7.01-7.04 (m, 1H), 5.46-5.50 (m, 2H), 5.30-5.34 (m, 2H), 3.68-3.78 (m, 4H), 3.44-3.53 (m, 6H), 3.24-3.30 (m, 1H), 3.07-3.15 (m, 1H), 2.45-2.49 (m, 1H), 2.20 (s, 7H), 1.53-1.61 (m, 2H), 1.42-1.48 (m, 11H), 1.20-1.32 (m, 4H), 0.92 (t, J=7.09 Hz, 3, 0.74-0.79 (m, 3H). LCMS: 746.5 [M+H]⁺.

### Step-3: Preparation of (S)-10-((Dimethylamino)methyl)-4-ethyl-4-(heptanoyloxy)-3,14-dioxo-3,4,12,14-tetrahydro-1H-pyrano[3′,4′:6,7]indolizino[1,2-b]quinolin-9-yl Piperazine-1-carboxylate (Int-3)

To a stirred solution of (S)-1-(tert-butyl) 4-(10-((dimethylamino)methyl)-4-ethyl-4-(heptanoyloxy)-3,14-dioxo-3,4,12,14-tetrahydro-1H-pyrano[3′,4′:6,7]indolizino[1,2-b]quinolin-9-yl)piperazine-1,4-dicarboxylate (Int-3, 5 g, 6.71 mmol, 1.0 eq.) in DCM (50 mL, 10 vol), TFA (5.1 mL, 67.1 mmol, 10.0 eq.) was added at ambient temperature. The reaction mixture was allowed to stir at room temperature until TLC indicated complete consumption of starting material. The reaction mixture was then evaporated under reduced pressure, diluted with DCM (500 mL) and washed with saturated bicarbonate solution (2×250 mL), brine (250 mL), dried over anhydrous sodium sulfate, filtered and concentrated under reduced pressure to obtain (S)-10-((dimethylamino)methyl)-4-ethyl-4-(heptanoyloxy)-3,14-dioxo-3,4,12,14-tetrahydro-1H-pyrano[3′,4′:6,7]indolizino[1,2-b]quinolin-9-yl piperazine-1-carboxylate (Int-3, 3 g, 69%) as a brown solid.

¹H NMR (400 MHz, DMSO-d₆) δ 8.94 (s, 1H), 8.04-8.10 (m, 1H), 7.61-7.67 (m, 1H), 7.00-7.06 (m, 1H), 5.45-5.51 (m, 2H), 5.28-5.35 (m, 2H), 3.56-3.81 (m, 4H), 3.36-3.46 (m, 2H), 2.73-2.89 (m, 4H), 2.52-2.57 (m, 1H), 2.44-2.48 (m, 1H), 2.14-2.21 (m, 8H), 1.51-1.61 (m, 2H), 1.17-1.34 (m, 7H), 0.89-0.95 (m, 3H), 0.74-0.79 (m, 3H). LCMS: 646.5 [M+H]⁺.

### Step-4: Preparation of (S)-10-((Dimethylamino)methyl)-4-ethyl-4-(heptanoyloxy)-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate

To a stirred solution of (S)-10-((dimethylamino)methyl)-4-ethyl-4-(heptanoyloxy)-3,14-dioxo-3,4,12,14-tetrahydro-1H-pyrano[3′,4′:6,7]indolizino[1,2-b]quinolin-9-yl piperazine-1-carboxylate (Int-3, 3.0 g, 4.65 mmol, 1.0 eq.) in MeOH (30 mL, 10 vol), (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-C, 2.59 g, 4.65 mmol) and acetic acid (1 mL, catalytic amount) were added at 0° C. under argon atmosphere. The reaction mixture was stirred at RT for 1 h and NaCNBH₃ (586 mg, 9.3 mmol) was added at 0° C. under argon atmosphere. The resulting reaction mixture was allowed to stir at room temperature until TLC indicated complete consumption of starting material. The reaction mixture was then quenched with saturated sodium bicarbonate solution (250 mL), extracted with ethyl acetate (2×500 mL), combined organic layer washed with brine solution (200 mL), dried over anhydrous sodium sulfate, filtered and concentrated under reduced pressure. The crude obtained was purified by Prep. HPLC (column: Spherical-C18, 40 uM, 100A; Mobile Phase A: 0.1% FA in water; Mobile Phase B: acetonitrile; flow rate: 25 mL/min, diluent: THF+DMSO). The pure fractions were combined and lyophilized under reduced pressure to obtain (S)-10-((dimethylamino)methyl)-4-ethyl-4-(heptanoyloxy)-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (1.51 g, 27%) as an off-white solid.

¹H NMR (400 MHz, DMSO-d₆) δ 8.94 (s, 1H), 8.07 (d, J=9.26 Hz, 1H), 7.63 (d, J=9.26 Hz, 1H), 6.96-7.04 (m, 3H), 6.56-6.62 (m, 2H), 5.67 (s, 1H), 5.48 (d, J=1.25 Hz, 2H), 5.32 (d, J=3.00 Hz, 2H), 4.40 (d, J=6.88 Hz, 1H), 3.67-3.79 (m, 4H), 3.44-3.49 (m, 2H), 3.25 (d, J=7.25 Hz, 3H), 2.64-2.83 (m, 6H), 2.53-2.60 (m, 3H), 2.31-2.48 (m, 6H), 2.08-2.22 (m, 15H), 1.86-2.01 (m, 5H), 1.65-1.74 (m, 2H), 1.41-1.62 (m, 7H), 1.18-1.36 (m, 12H), 0.92 (t, J=7.44 Hz, 3H), 0.74-0.79 (m, 3H), 0.24 (s, 3H). LCMS: 1189.1 [M+H]⁺.

### Example S13. Preparation of (S)-10-((4-(1-(6-(((1r,4r)-4-(3-Chloro-4-cyanophenoxy)cyclohexyl)carbamoyl)pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl [1,4′-bipiperidine]-1′-carboxylate (Compound No. 13)

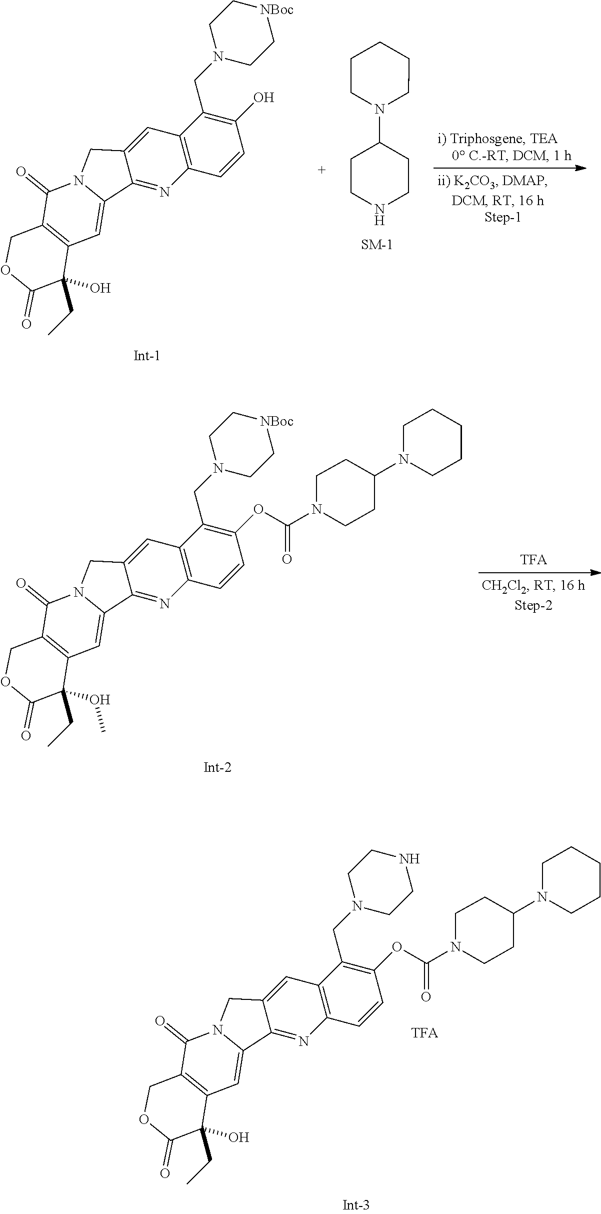

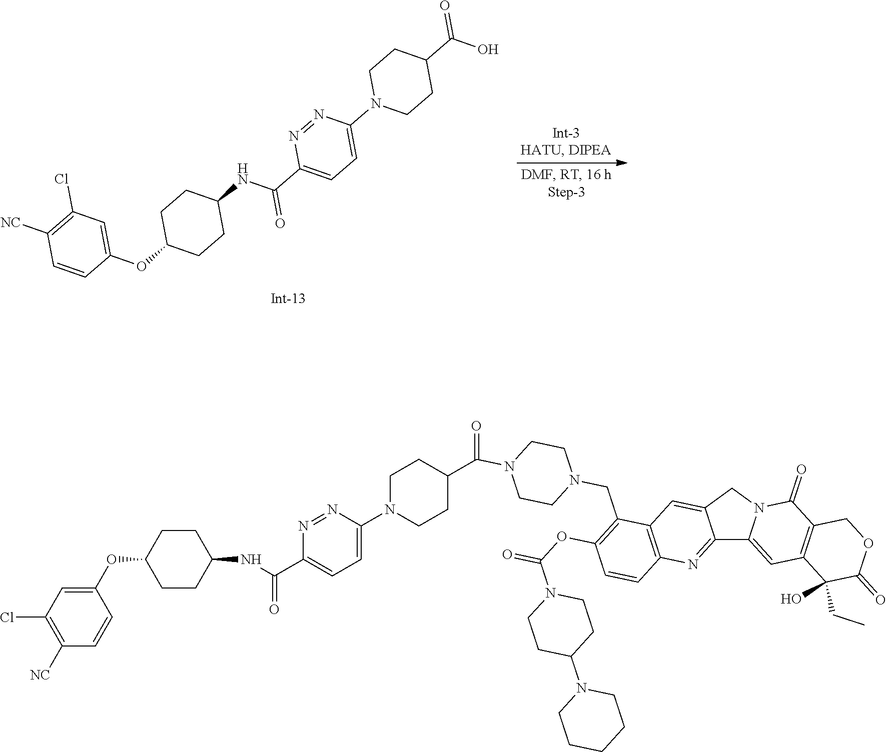 [SMILES: N#Cc1ccc(O[C@H]2CC[C@H](NC(=O)c3ccc(N4CCC(C(=O)O)CC4)nn3)CC2)cc1Cl ; CC[C@@]1(O)C(=O)OCc2c1cc1n(c2=O)Cc2cc3c(CN4CCN(C(=O)C5CCN(c6ccc(C(=O)N[C@H]7CC[C@H](Oc8ccc(C#N)c(Cl)c8)CC7)nn6)CC5)CC4)c(OC(=O)N4CCC(N5CCCCC5)CC4)ccc3nc2-1]

### Step-1: Preparation of (S)-10-((4-(tert-Butoxycarbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl [1,4′-bipiperidine]-1′-carboxylate (Int-2)

To a stirred solution of SM-1 (60 mg, 0.35 mmol, 1.0 eq.) in DCM (10 mL) were added triethylamine (0.14 mL, 1.06 mmol, 3 eq.) and triphosgene (105 mg, 0.35 mmol, 1 eq.) at 0° C. The reaction mixture was allowed to warm up to room temperature and stir for 1 h. After 1 h, Int-1 (200 mg, 0.35 mmol, 1 eq.), K₂CO₃ (49 mg, 0.35 mmol, 1 eq.) and cat. Amount of DMAP (20 mg) were added to this reaction mixture and stirring was continued for 16 h. Progress of the reaction was monitored by TLC. After reaction completion, the mixture was diluted with water (100 mL) and extracted with DCM (2×200 mL). The combined organic extract was washed with water (100 mL), brine (100 mL), dried over anhydrous sodium sulfate, filtered and concentrated under vacuum to obtain the crude product. The crude obtained was purified by combiflash column using 4% methanol in DCM to afford Int-2 (200 mg, 74%) as an off white solid.

¹H NMR (400 MHz, DMSO-d₆) δ 9.01 (s, 1H), 8.11 (br d, J=9.29 Hz, 1H), 7.63 (br d, J=9.29 Hz, 1H), 7.34 (s, 1H), 6.51 (br s, 1H), 5.43 (br s, 2H), 5.30 (br s, 2H), 4.26-4.28 (m, 1H), 4.06-4.08 (m, 3H), 3.86 (br s, 2H), 3.25-3.28 (m, 6H), 3.16-3.18 (m, 4H), 2.85-2.99 (m, 1H), 2.39-2.43 (m, 4H), 1.80-1.89 (m, 6H), 1.35-1.46 (m, 4H), 1.32 (s, 9H), 0.89 (br t, J=7.34 Hz, 3H). LCMS: 757.4 [M+H]⁺.

### Step-2: Preparation of (S)-4-Ethyl-4-hydroxy-3,14-dioxo-10-(piperazin-1-ylmethyl)-3,4,12,14-tetrahydro-1H-pyrano[3′,4′:6,7]indolizino[1,2-b]quinolin-9-yl [1,4′-bipiperidine]-1′-carboxylate (Int-3)

To a stirred solution of Int-2 (200 mg, 0.26 mmol, 1.0 eq.) in DCM (10 mL) under nitrogen atmosphere was added TFA (0.2 mL, 2.64 mmol, 10 eq.) at 0° C. The reaction mixture was allowed to warm up to room temperature and stir for 16 h. Progress of the reaction was monitored by TLC. After completion of the reaction, solvents were evaporated under reduced pressure, washed with diethyl ether (25 mL) and pentane (20 mL) and dried under vacuum to afford Int-3 (170 mg, 98%) as an off white solid.

¹H NMR (400 MHz, DMSO-d₆) δ 9.73 (br d, J=8.80 Hz, 1H), 9.03 (s, 1H), 8.82 (br s, 2H), 8.16 (br d, J=9.29 Hz, 1H), 7.66 (br d, J=9.29 Hz, 1H), 7.34 (s, 1H), 5.44 (s, 2H), 5.31 (s, 2H), 4.39-4.42 (m, 1H), 4.13-4.28 (m, 1H), 3.96 (br s, 2H), 3.47 (br d, J=10.76 Hz, 2H), 2.84-3.27 (m, 8H), 2.67-2.71 (m, 4H), 2.14-2.18 (m, 2H), 1.61-2.08 (m, 10H), 1.44 (br d, J=12.72 Hz, 1H), 0.89 (t, J=7.34 Hz, 3H). LCMS: 657.20 [M+H]⁺.

### Step-3: Preparation of (S)-10-((4-(1-(6-(((1r,4r)-4-(3-Chloro-4-cyanophenoxy)cyclohexyl)carbamoyl)pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl [1,4′-bipiperidine]-1′-carboxylate

To a stirred solution of Int-3 (170 mg, 0.25 mmol, 1.0 eq.) and Int-13 (125 mg, 0.25 mmol, 1.0 eq.) in DMF (5 mL) were added HATU (185 mg, 0.51 mmol, 2.0 eq.) and DIPEA (0.13 mL, 0.77 mmol, 3 eq.) at RT and the reaction mixture was allowed to stir for 16 h. Progress of the reaction was monitored by TLC. After completion of the reaction, the reaction mixture was diluted with water (100 mL) and extracted with ethyl acetate (2×100 mL). The combined organic extract was washed with water (100 mL), brine (100 mL) and dried over anhydrous sodium sulfate. Solvents were evaporated under reduced pressure to obtain the crude product. The crude was purified by combiflash column eluting with 13% methanol in DCM to afford the title compound (108 mg, 37%) as an off-white solid.

¹H NMR (400 MHz, DMSO-d₆) δ 9.04 (s, 1H), 8.57 (br d, J=8.13 Hz, 1H), 8.43 (s, 1H), 8.13 (d, J=9.13 Hz, 1H), 7.81-7.89 (m, 1H), 7.64 (d, J=9.13 Hz, 1H), 7.30-7.40 (m, 3H), 7.13 (dd, J=8.82, 2.31 Hz, 1H), 6.55 (s, 1H), 5.43 (s, 2H), 5.31 (s, 2H), 4.42-4.59 (m, 3H), 4.24-4.36 (m, 1H), 4.02-4.13 (m, 1H), 3.81-3.93 (m, 3H), 3.50-3.52 (m, 2H), 3.10-3.13 (m, 3H), 2.87-3.05 (m, 3H), 2.54-2.57 (m, 3H), 2.46-2.50 (m, 4H), 2.40-2.42 (m, 3H), 2.08-2.11 (m, 2H), 1.80-1.93 (m, 6H), 1.42-1.74 (m, 14H), 1.34-1.37 (m, 2H), 0.89 (br t, J=7.32 Hz, 3H). LCMS: 1122.4 [M+H]⁺.

HPLC purity 92.4%.

### Example S14. Preparation of (S)-10-((Dimethylamino)methyl)-4-((dimethylglycyl)oxy)-4-ethyl-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Compound No. 14)

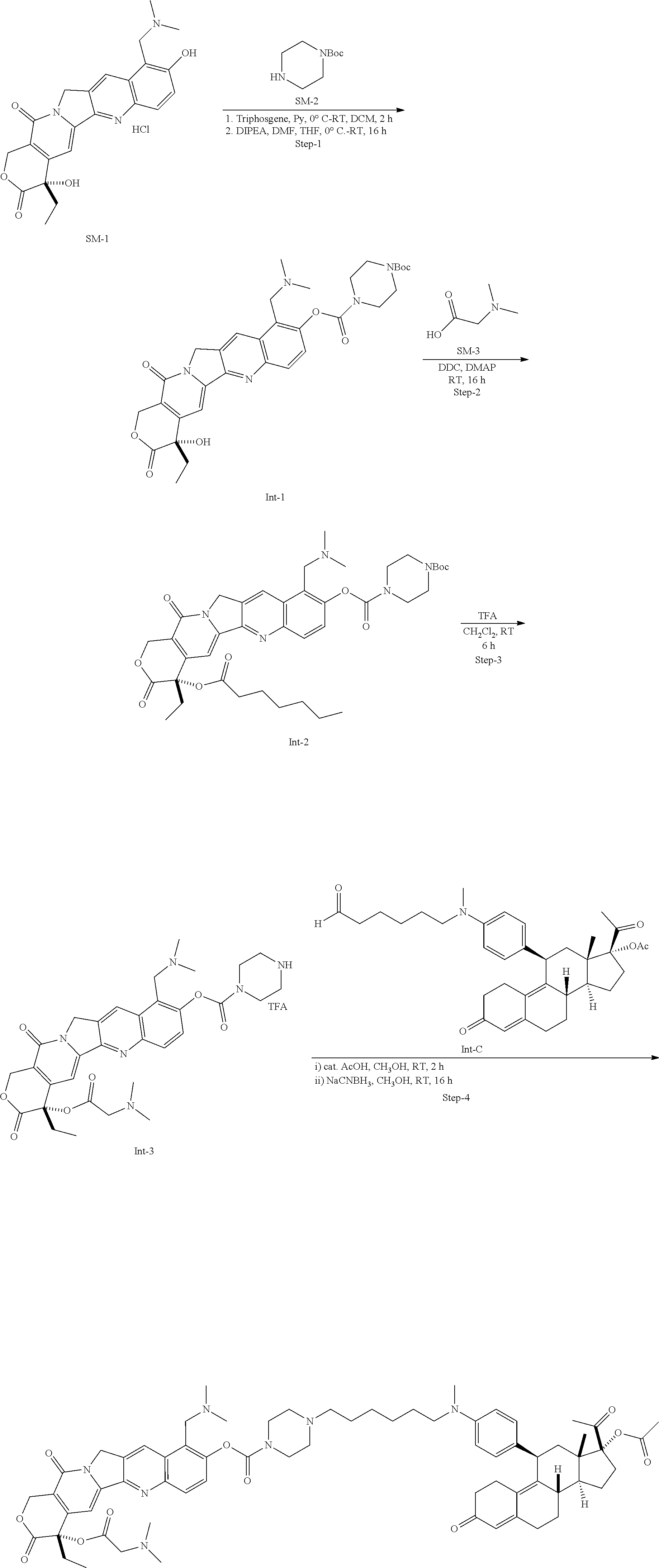

### Step-1-1: Preparation of tert-Butyl 4-(Chlorocarbonyl)piperazine-1-carboxylate

To a stirred solution of tert-butyl piperazine-1-carboxylate (SM-2, 5 g, 26.8 mmol, 1.0 eq.) in DCM (100 mL) were added pyridine (2.97 g, 37.6 mmol, 1.4 eq.) and triphosgene (3.19 g, 10.7 mmol, 0.4 eq.) at 0° C. The reaction mixture was allowed to warm up to room temperature and stir for 2 h. Progress of the reaction was monitored by TLC. After completion of starting material, the reaction mixture was washed with water (100 mL) and extracted with DCM (2×100 mL). The combined organic extract was again washed with water (200 mL), brine (200 mL) and dried over anhydrous sodium sulfate, filtered, and concentrated under vacuum to afford tert-butyl 4-(chlorocarbonyl)piperazine-1-carboxylate (6.0 g, 90%) as a crude oil.

¹H NMR (400 MHz, DMSO-d₆) δ 3.64 (br d, J=4.40 Hz, 2H), 3.52 (br s, 2H), 3.27-3.47 (m, 3H), 2.93-3.21 (m, 1H), 1.41 (s, 9H).

### Step-1-2: Preparation of (S)-1-(tert-Butyl) 4-(10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)piperazine-1,4-dicarboxylate (Int-1)

To a stirred solution of (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione hydrochloride (SM-1, 10 g, 23.7 mmol, 1.0 eq.) in DCM (100 mL, 10 vol) were added DIPEA (15.3 g, 118 mmol, 5 eq.) and DMAP (724 mg, 5.9 mmol, 0.25 eq.) followed by addition of tert-butyl 4-(chlorocarbonyl)piperazine-1-carboxylate (5.89 g, 23.7 mmol, 1 eq.) in DCM (100 mL, 10 vol) dropwise over a period of 10 min at 0° C. The resulting reaction mixture was allowed to warm up to room temperature and stir for 16 h. Progress of the reaction was monitored by TLC. After completion of the reaction, the mixture was washed with water (500 mL), extracted with DCM (3×100 mL) and the combined organic extract was again washed with water (200 mL), brine (200 mL) and dried over anhydrous sodium sulfate, filtered and concentrated under vacuum. The crude obtained was purified by combiflash column eluting with 7% methanol in DCM to afford Int-1 (10 g, 66%) as an off-white solid.

¹H NMR (400 MHz, DMSO-d₆) δ 8.95 (s, 1H), 8.11 (d, J=9.29 Hz, 1H), 7.65 (d, J=9.29 Hz, 1H), 7.34 (s, 1H), 6.53 (s, 1H), 5.43 (s, 2H), 5.31 (s, 2H), 3.67-3.79 (m, 4H), 3.41-3.55 (m, 6H), 2.20 (s, 6H), 1.80-1.93 (m, 2H), 1.44 (s, 9H), 0.89 (br t, J=7.34 Hz, 3H). LCMS: 634.2 [M+H]⁺.

### Step-2: Preparation of (S)-1-(tert-Butyl) 4-(10-((Dimethylamino)methyl)-4-((dimethylglycyl)oxy)-4-ethyl-3,14-dioxo-3,4,12,14-tetrahydro-1H-pyrano[3′,4′:6,7]indolizino[1,2-b]quinolin-9-yl) Piperazine-1,4-dicarboxylate (Int-2)

To a stirred solution of (S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) piperazine-1,4-dicarboxylate (Int-1, 7.0 g, 11.05 mmol, 1.0 eq.) in DCM (70 mL, 10.0 vol), dimethylglycine (SM-3, 1.7 g, 16.57 mmol, 1.5 eq.), DCC (3.41 g, 16.57 mmol, 1.5 eq.) and DMAP (134 mg, 1.105 mmol, 0.1 eq.) were added at 0° C. under argon atmosphere. The reaction mixture was allowed to stir at ambient temperature until TLC indicated complete consumption of starting material. The reaction mixture was then diluted with ice cold water (250 mL) and extracted with DCM (2×250 mL). The combined organic layer was washed with brine solution (200 mL), dried over anhydrous sodium sulfate, filtered and concentrated under reduced pressure. The crude obtained was purified by flash column and the pure fractions combined and concentrated under reduced pressure to afford(S)-1-(tert-butyl) 4-(10-((dimethylamino)methyl)-4-((dimethylglycyl)oxy)-4-ethyl-3,14-dioxo-3,4,12,14-tetrahydro-1H-pyrano[3′,4′:6,7]indolizino[1,2-b]quinolin-9-yl)piperazine-1,4-dicarboxylate (Int-2, 6 g, 75%) as an off-white solid.

¹H NMR (400 MHz, DMSO-d₆) δ 8.95 (s, 1H), 8.09 (d, J=8.31 Hz, 1H), 7.65 (d, J=9.29 Hz, 1H), 7.06 (s, 1H), 5.41-5.59 (m, 2H), 5.26-5.39 (m, 2H), 3.76 (s, 2H), 3.65-3.73 (m, 2H), 3.40-3.56 (m, 5H), 3.23-3.31 (m, 1H), 3.07-3.15 (m, 1H), 2.94 (s, 2H), 2.24-2.28 (m, 4H), 2.19-2.22 (m, 4H), 2.11-2.18 (m, 1H), 1.55-1.76 (m, 1H), 1.44 (s, 9H), 1.40 (s, 3H), 0.93 (t, J=7.09 Hz, 3H). LCMS: 719.5 [M+H]⁺.

### Step-3: Preparation of (S)-10-((Dimethylamino)methyl)-4-((dimethylglycyl)oxy)-4-ethyl-3,14-dioxo-3,4,12,14-tetrahydro-1H-pyrano[3′,4′:6,7]indolizino[1,2-b]quinolin-9-yl Piperazine-1-carboxylate (Int-3)

To a stirred solution of (S)-1-(tert-butyl) 4-(10-((dimethylamino)methyl)-4-((dimethylglycyl)oxy)-4-ethyl-3,14-dioxo-3,4,12,14-tetrahydro-1H-pyrano[3′,4′:6,7]indolizino[1,2-b]quinoline-9-yl)piperazine-1,4-dicarboxylate (Int-2, 5 g, 6.96 mmol, 1.0 eq.) in DCM (50 mL, 10 vol), TFA (5.32 mL, 69.6 mmol, 10.0 eq.) was added at ambient temperature. The reaction mixture was allowed to stir at room temperature until TLC indicated complete consumption of starting material. The reaction mixture was then concentrated under reduced pressure, diluted with DCM (500 mL), washed with saturated bicarbonate solution (2×250 mL), brine (250 mL), dried over anhydrous sodium sulfate, filtered and concentrated under reduced pressure to obtain (S)-10-((dimethylamino)-methyl)-4-((dimethylglycyl)oxy)-4-ethyl-3,14-dioxo-3,4,12,14-tetrahydro-1H-pyrano[3′,4′:6,7]indolizino[1,2-b]quinolin-9-yl piperazine-1-carboxylate (Int-3, 2.2 g, 51%) as a pale yellow solid.

¹H NMR (400 MHz, DMSO-d₆) δ 8.94 (s, 1H), 8.08 (d, J=9.26 Hz, 1H), 7.63 (d, J=9.26 Hz, 1H), 7.06 (s, 1H), 5.50 (s, 2H), 5.32 (s, 2H), 3.76 (s, 2H), 3.63 (s, 2H), 3.37-3.47 (m, 4H), 2.72-2.87 (m, 5H), 2.55-2.64 (m, 1H), 2.26 (s, 5H), 2.20 (s, 6H), 2.16 (dd, J=7.44, 2.19 Hz, 2H), 0.93 (t, J=7.38 Hz, 3H). LCMS: 619.3 [M+H]⁺.

### Step-4: Preparation of (S)-10-((Dimethylamino)methyl)-4-((dimethylglycyl)oxy)-4-ethyl-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate

To a stirred solution of (S)-10-((dimethylamino)methyl)-4-((dimethylglycyl)oxy)-4-ethyl-3,14-dioxo-3,4,12,14-tetrahydro-1H-pyrano[3′,4′:6,7]indolizino[1,2-b]quinolin-9-yl piperazine-1-carboxylate (Int-3, 2.5 g, 4.04 mmol, 1.0 eq.) in McOH (25 mL, 10 vol), (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-C, 2.26 g, 4.04 mmol) and acetic acid (1 mL, catalytic amount) was added at 0° C. under argon atmosphere. The reaction mixture was allowed to stir at RT for 1 h and NaCNBH₃ (509 mg, 8.08 mmol) was then added at 0° C. under argon atmosphere. The resulting reaction mixture was allowed to stir at room temperature until TLC indicated complete consumption of starting material. The reaction mixture was quenched with saturated sodium bicarbonate solution (250 mL), extracted with ethyl acetate (2×500 mL). The combined organic layer was washed with brine solution (200 mL), dried over anhydrous sodium sulfate, filtered and concentrated under reduced pressure. The crude obtained was purified by Prep. HPLC (column: spherical-C18, 40 uM, 100A; Mobile Phase A: 0.1% FA in water; Mobile Phase B: acetonitrile; flow rate: 25 mL/min, diluent: THF+DMSO). The pure fractions were combined and lyophilized under reduced pressure to obtain (S)-10-((dimethylamino)methyl)-4-((dimethylglycyl)oxy)-4-ethyl-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (663 mg, 14%) as an off-white solid.

¹H NMR (400 MHz, DMSO-d₆) δ 8.95 (s, 1H), 8.09 (d, J=9.29 Hz, 1H), 7.63 (d, J=9.29 Hz, 1H), 7.06 (s, 1H), 6.99 (d, J=8.31 Hz, 2H), 6.59 (d, J=8.31 Hz, 2H), 5.67 (s, 1H), 5.50 (s, 2H), 5.30-5.35 (m, 2H), 4.38-4.43 (m, 1H), 3.67-3.77 (m, 4H), 3.37-3.47 (m, 6H), 3.22-3.27 (m, 4H), 2.82 (s, 3H), 2.61-2.82 (m, 3H), 2.53-2.58 (m, 2H), 2.41 (s, 2H), 2.30-2.37 (m, 3H), 2.26 (s, 5H), 2.16-2.21 (m, 10H), 2.10 (s, 4H), 2.00 (s, 4H), 1.86-1.98 (m, 2H), 1.62-1.77 (m, 1H), 1.39-1.52 (m, 5H), 1.26-1.36 (m, 5H), 0.90-0.95 (m, 3H), 0.24 (s, 3H). LCMS: 1162.1 [M+H]⁺.

### Example S15. Preparation of N-((1r,4r)-4-((3-Chloro-4-cyanophenyl)(methyl)amino)cyclohexyl)-6-(4-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)-piperazine-1-carbonyl)piperidin-1-yl)pyridazine-3-carboxamide (Compound No. 15)

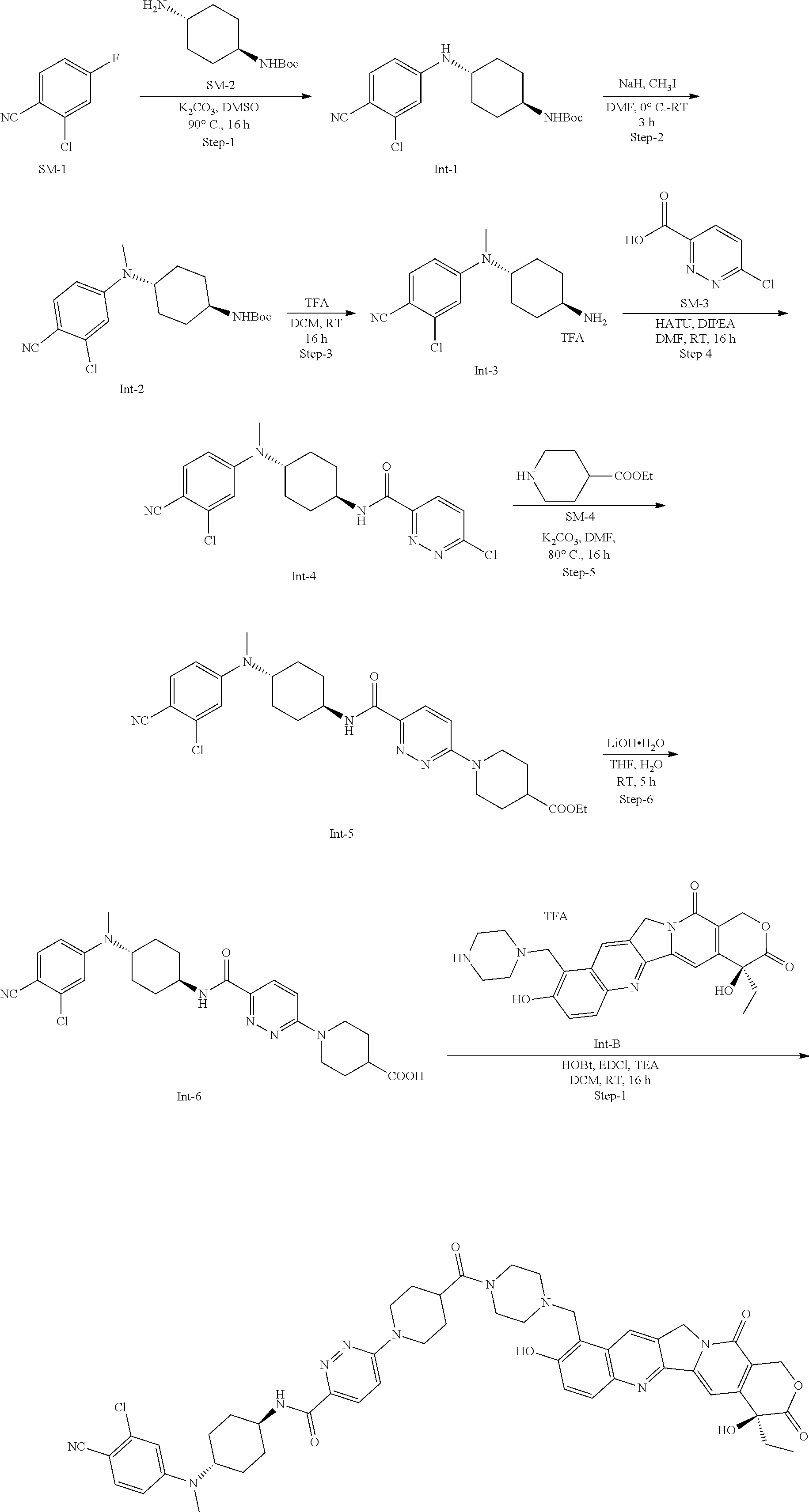

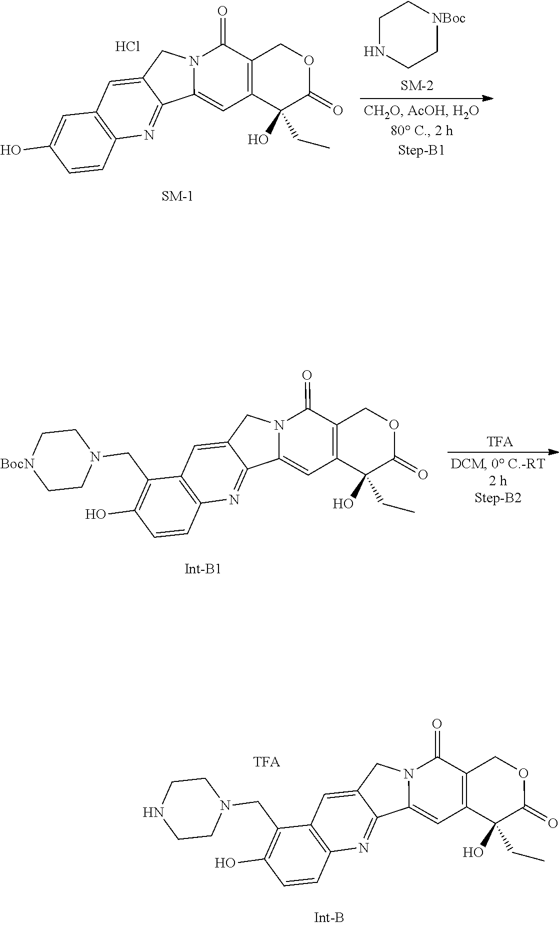 [SMILES: CC[C@@]1(O)C(=O)OCc2c1cc1n(c2=O)Cc2cc3cc(O)ccc3nc2-1 ; Cl ; CC(C)(C)OC(=O)N1CCNCC1 ; CC[C@@]1(O)C(=O)OCc2c1cc1n(c2=O)Cc2cc3c(CN4CCN(C(=O)OC(C)(C)C)CC4)c(O)ccc3nc2-1 ; CC[C@@]1(O)C(=O)OCc2c1cc1n(c2=O)Cc2cc3c(CN4CCNCC4)c(O)ccc3nc2-1]

### Preparation of (S)-4-Ethyl-4,9-dihydroxy-10-(piperazin-1-ylmethyl)-1,12-dihydro-14H-pyrano[3′,4′:6,7]-indolizino[1,2-b]quinoline-3,14(4H)-dione Trifluoroacetate (Int-B)

### Step-B1: Preparation of tert-Butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-B1)

To a stirred solution of (S)-4-ethyl-4,9-dihydroxy-1,12-dihydro-14H-pyrano[3′,4′:6,7]indolizino[1,2-b]quinoline-3,14(4H)-dione HCl salt (SM-1, 2.5 g, 6.86 mmol, 1.0 eq.) and tert-butyl piperazine-1-carboxylate (SM-2, 1.85 g, 10.3 mmol, 1.5 eq.) in acetic acid (10 mL) under inert atmosphere was added 37% solution of formaldehyde (0.29 mL, 8.24 mmol, 1.2 eq.) at room temperature. The reaction mixture was heated to 80° C. for 2 h in a sealed tube. Progress of the reaction was monitored by TLC. After completion of the reaction, solvents were evaporated under reduced pressure to obtain the crude product, which was basified with aq. ammonia until pH 9. The resulting solid was filtered, washed with water (10 mL) and dried under vacuum to afford Int-B1 (1.9 g, 50%) as a yellow solid.

LCMS: 463.46 [M-100]⁺ (Boc group cleavage was observed in LCMS).

### Step-B2: Preparation of (S)-4-Ethyl-4,9-dihydroxy-10-(piperazin-1-ylmethyl)-1,12-dihydro-14H-pyrano-[3′,4′:6,7]indolizino[1,2-b]quinoline-3,14(4H)-dione Trifluoroacetate Salt (Int-B)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-B1, 2 g, 3.5 mmol, 1.0 eq.) in DCM (20 mL) under nitrogen atmosphere was added TFA (2.7 mL, 35 mmol, 10 eq.) at 0° C. The reaction mixture was warmed up to room temperature and stirred for 2 h. Progress of the reaction was monitored by TLC. After completion of the reaction, solvents were evaporated under reduced pressure, washed the resulting residue with diethyl ether (10 mL) and dried under vacuum to afford Int-B (1.4 g, 83%) as a yellow solid.

¹H NMR (400 MHz, DMSO-d₆) δ 8.86 (br s, 2H), 8.10 (d, J=9.25 Hz, 1H), 7.58 (d, J=9.25 Hz, 1H), 7.21-7.32 (m, 2H), 6.97-7.15 (m, 1H), 6.34-6.64 (m, 1H), 5.42 (s, 2H), 5.26 (s, 2H), 4.40 (s, 2H), 3.10-3.30 (m, 8H), 1.85-1.89 (m, 2H), 0.88 (t, J=7.17 Hz, 3H).

### Preparation of N-((1r,4r)-4-((3-Chloro-4-cyanophenyl)(methyl)amino)cyclohexyl)-6-(4-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)-piperazine-1-carbonyl)piperidin-1-yl)pyridazine-3-carboxamide

To a stirred solution of 1-(6-(((1r,4r)-4-((3-chloro-4-cyanophenyl)(methyl)amino)cyclohexyl)carbamoyl)-pyridazin-3-yl)piperidine-4-carboxylic acid (Int-6 of Example S11, 300 mg, 0.60 mmol, 1.0 eq.) and (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione trifluoro-acetate salt (Int-B, 278 mg, 0.60 mmol, 1.0 eq.) in DCM (10 mL) were added HOBt (138 mg, 0.90 mmol, 1.5 eq.), EDCI·HCl (173 mg, 0.90 mmol, 1.5 eq.) and DIPEA (155.7 mg, 1.20 mmol, 2 eq.) at room temperature and stirred for 16 h. Progress of the reaction was monitored by TLC. After completion of the reaction, water (50 mL) was added and the aqueous solution was extracted with DCM (2×100 mL). The combined organic extract was washed with water (100 mL), brine (100 mL) and dried over anhydrous sodium sulfate. Solvents were evaporated under reduced pressure to obtain the crude product which was purified by prep. HPLC using ammonium bicarbonate in water and acetonitrile as a mobile phase to afford the title compound (60 mg, 10%) as an off-white solid.

¹H NMR (400 MHz, DMSO-d₆) δ 8.77 (s, 1H), 8.48 (d, J=8.26 Hz, 1H), 7.99 (d, J=9.26 Hz, 1H), 7.82 (d, J=9.51 Hz, 1H), 7.60 (d, J=9.01 Hz, 1H), 7.47 (d, J=9.13 Hz, 1H), 7.35 (d, J=9.76 Hz, 1H), 7.26 (s, 1H), 6.94 (d, J=2.38 Hz, 1H), 6.83 (dd, J=9.07, 2.44 Hz, 1H), 6.48 (s, 1H), 5.42 (s, 2H), 5.26 (s, 2H), 4.48 (br d, J=13.01 Hz, 2H), 4.03 (s, 2H), 3.75-3.88 (m, 2H), 3.41-3.62 (m, 3H), 2.96-3.16 (m, 3H), 2.85 (s, 3H), 2.51-2.54 (m, 2H), 1.83-1.97 (m, 5H), 1.51-1.79 (m, 12H), 0.88 (t, J=7.32 Hz, 3H). (1H exchangeable hydrogen was not observed in spectra).

LCMS: 941.4 [M+H]⁺. HPLC: 94.2%.

### Example S16. Preparation of N-((1r,4r)-4-(3-Chloro-4-cyanophenoxy)cyclohexyl)-6-(4-(4-((((S)-10-((dimethylamino)-methyl)-4-ethyl-4-hydroxy-3,14-dioxo-3,4,12,14-tetrahydro-1H-pyrano[3′,4′:6,7]indolizino[1,2-b]quinolin-9-yl)oxy)methyl)piperidine-1-carbonyl)piperidin-1-yl)pyridazine-3-carboxamide (Compound No. 16)

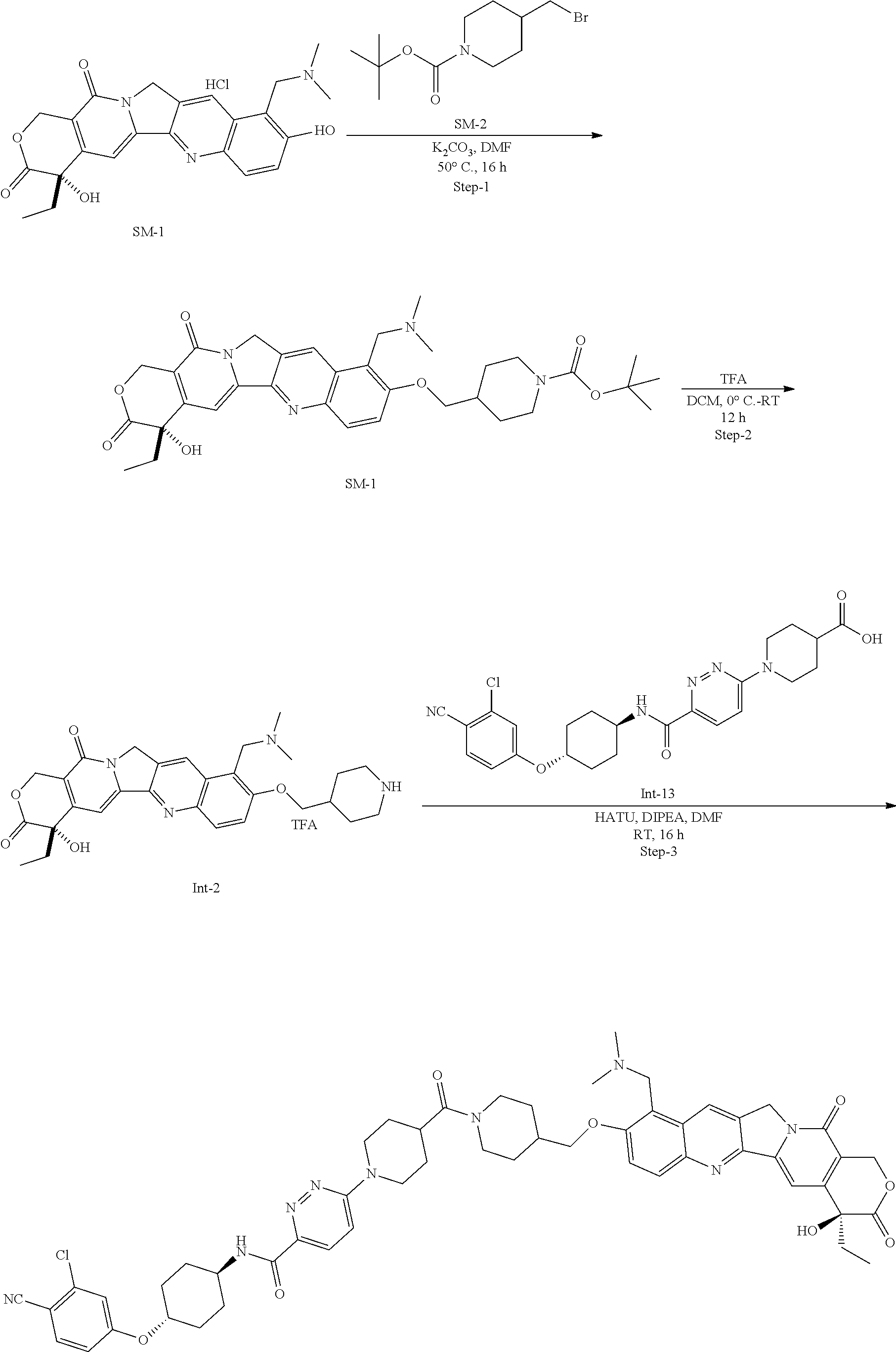

### Step-1: Preparation of tert-Butyl (S)-4-(((10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)oxy)methyl)piperidine-1-carboxylate (Int-1)

To a stirred solution of (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione HCl salt (SM-1, 2 g, 4.75 mmol, 1.0 eq.) in DMF (10 mL) under nitrogen atmosphere were added K₂CO₃ (1.31 g, 9.5 mmol, 2.0 eq.) and tert-butyl 4-(bromomethyl)piperidine-1-carboxylate (SM-2, 1.71 g, 6.17 mmol, 1.3 eq.) at room temperature. The reaction mixture was heated to 50° C. 16 h. Progress of the reaction was monitored by TLC. After completion of the reaction, the reaction mixture was diluted in ice cold water (20 mL) and extracted with ethyl acetate (2×50 mL). The combined organic extract was washed with brine (100 mL), dried over anhydrous sodium sulfate, filtered and concentrated under vacuum to obtain Int-1 (1.0 g, crude) as a yellow solid which was used in next step without further purification.

LCMS: 617.2 [M−H]⁻.

### Step-2: Preparation of (S)-10-((dimethylamino)methyl)-4-ethyl-4-hydroxy-9-(piperidin-4-ylmethoxy)-1,12-dihydro-14H-pyrano[3′,4′:6,7]indolizino[1,2-b]quinoline-3,14(4H)-dione Trifluoroacetate Salt (Int-2)

To a stirred solution of Int-1 (1.0 g, 1.41 mmol, 1.0 eq.) in DCM (50 mL) was added TFA (10 mL) at 0° C. The reaction mixture was allowed to warm up to room temperature and stir for 12 h. Progress of the reaction was monitored by TLC. After completion of the reaction, solvents were evaporated under reduced pressure to obtain the crude product which was triturated with diethyl ether:pentane (80:20) three times (25 mL each) and dried under vacuum to afford Int-2 (800 mg, crude) as pale-yellow solid which was used in next step without further purification.

LCMS: 519.26 [M+H]⁺.

### Preparation of N-((1r,4r)-4-(3-Chloro-4-cyanophenoxy)cyclohexyl)-6-(4-(4-((((S)-10-((dimethylamino)-methyl)-4-ethyl-4-hydroxy-3,14-dioxo-3,4,12,14-tetrahydro-1H-pyrano[3′,4′:6,7]indolizino[1,2-b]quinolin-9-yl)oxy)methyl)piperidine-1-carbonyl)piperidin-1-yl)pyridazine-3-carboxamide

To a stirred solution of (S)-10-((dimethylamino)methyl)-4-ethyl-4-hydroxy-9-(piperidin-4-ylmethoxy)-1,12-dihydro-14H-pyrano[3′,4′:6,7]indolizino[1,2-b]quinoline-3,14(4H)-dione (Int-2, 370 mg, 0.772 mmol, 1.0 eq.) in DMF (3 mL) were added DIPEA (0.27 mL, 1.54 mmol, 2.0 eq.) and HATU (440 mg, 1.15 mmol, 1.5 eq.). After 5-10 minutes, Int-13 (400 mg, 0.772 mmol, 1.0 eq.) was added at room temperature and stirring was continued for an additional 16 h. Progress of the reaction was monitored by TLC. After completion of the reaction, solvents were evaporated, the resulting residue triturated with diethyl ether (20 mL), filtered, dried and purified by prep. HPLC eluting with Mobile phase A: 0.1% TFA in water and Mobile phase B: Acetonitrile to afford the title compound (50 mg, 6%) as a white solid.

¹H NMR (400 MHz, DMSO-d₆) δ 8.81 (s, 1H), 8.58 (d, J=8.13 Hz, 1H), 8.31 (s, 2H), 8.07-8.16 (m, 1H), 7.69-7.90 (m, 2H), 7.29-7.41 (m, 2H), 7.07-7.22 (m, 1H), 6.40-6.54 (m, 1H), 5.37-5.45 (m, 2H), 5.21-5.31 (m, 2H), 4.43-4.58 (m, 4H), 4.05-4.23 (m, 3H), 3.86 (br s, 3H), 3.06-3.20 (m, 5H), 2.62 (br s, 1H), 2.21 (s, 6H), 2.07-2.14 (m, 2H), 1.79-1.99 (m, 6H), 1.70-1.79 (m, 2H), 1.47-1.70 (m, 6H), 1.39 (d, J=10.88 Hz, 1H), 1.21-1.30 (m, 1H), 0.82-0.95 (m, 3H).

LCMS: 984.3 [M+H]⁺. HPLC purity 96.3%.

### Example S17. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-(1-(6-(((1r,4r)-4-((3-chloro-4-cyanophenyl)(methyl)-amino)cyclohexyl)carbamoyl)pyridazin-3-yl)piperidine-4-carbonyl)-2,5-dimethylpiperazine-1-carboxylate (Compound No. 17)

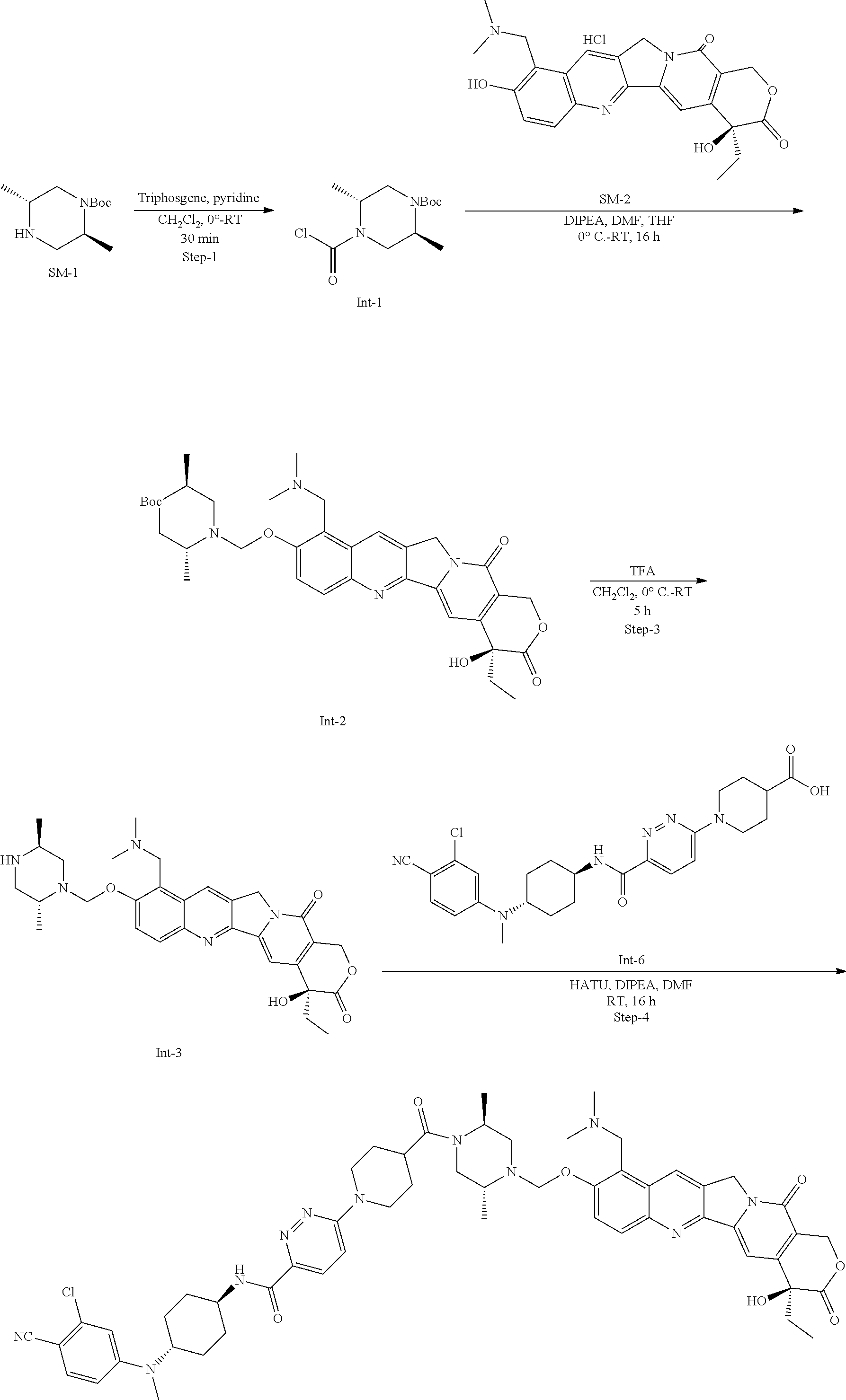

### Step-1: Preparation of tert-Butyl (2S,5R)-4-(Chlorocarbonyl)-2,5-dimethylpiperazine-1-carboxylate (Int-1)

To a stirred solution of tert-butyl (2S,5R)-2,5-dimethylpiperazine-1-carboxylate (SM-1, 3.0 g, 13.99 mmol, 1.0 eq.) in DCM (25 mL) were added pyridine (1.7 mL, 20.99 mmol, 1.5 eq.) and triphosgene (1.24 g, 41.99 mmol, 0.3 eq.) solution in DCM (5 mL) dropwise over a period of 10 min at 0° C. The reaction mixture was allowed to warm up to room temperature and stir for 30 min. Progress of the reaction was monitored by TLC (non-polar spot was observed). After completion of the reaction, the reaction mixture was poured into ice cold water (50 mL) and extracted with DCM (2×30 mL). The combined organic extract was washed with brine (100 mL), dried over anhydrous sodium sulfate, filtered, and concentrated under vacuum to afford Int-1 (3.7 g, crude) as a pale brown gum which was used in next step without further purification.

¹H NMR (400 MHz, DMSO-d₆) δ 4.09-4.55 (m, 2H), 3.46-3.69 (m, 2H), 3.13-3.41 (m, 2H), 1.40 (s, 9H), 0.98-1.28 (m, 6H).

### Step-2: Preparation of 1-(tert-Butyl) 4-((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) (2S,5R)-2,5-Dimethylpiperazine-1,4-dicarboxylate (Int-2)

To a solution of tert-butyl (2S,5R)-4-(chlorocarbonyl)-2,5-dimethylpiperazine-1-carboxylate (Int-1, 3.6 g, 13.05 mmol, 2 eq.) and (S)-10-((dimethylamino)methyl)-4-ethyl-4,9-dihydroxy-1,12-dihydro-14H-pyrano-[3′,4′:6,7]indolizino[1,2-b]quinoline-3,14(4H)-dione hydrochloride (SM-2, 3.1 g, 6.52 mmol, 1 eq.) in THF (30 mL) and DMF (30 mL) was added DIPEA (5.67 mL, 32.6 mmol, 5 eq.) at 0° C. The reaction mixture was allowed to warm up to room temperature and stir for 16 h. Progress of the reaction was monitored by TLC. After reaction completion, the reaction mixture was quenched with water (30 mL) and extracted with ethyl acetate (2×25 mL). The combined organic extract was washed with brine (100 mL), dried over anhydrous sodium sulfate, filtered and concentrated under vacuum to obtain the crude product. The crude obtained was purified by combiflash column chromatography eluting with 10% methanol in DCM to afford Int-2 (2.5 g, 58%) as a pale yellow foam.

¹H NMR (400 MHz, DMSO-d₆) δ 8.95 (br s, 1H), 8.11 (d, J=9.29 Hz, 1H), 7.54-7.76 (m, 1H), 7.34 (s, 1H), 6.53 (s, 1H), 5.43 (s, 2H), 5.31 (s, 2H), 4.19-4.41 (m, 2H), 3.61-3.81 (m, 2H), 3.17 (d, J=12.91 Hz, 1H), 2.20 (d, J=6.36 Hz, 6H), 1.80-1.95 (m, 3H), 1.26-1.41 (m, 14H), 1.18 (d, J=6.36 Hz, 3H), 0.89 (t, J=6.36 Hz, 3H). LCMS: 662.4 [M+H]⁺.

### Step-3: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-Dimethylpiperazine-1-carboxylate (Int-3)

To a stirred solution of 1-(tert-butyl) 4-((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) (2S,5R)-2,5-dimethylpiperazine-1,4-dicarboxylate (Int-2, 1.5 g, 2.26 mmol, 1.0 eq.) in DCM (20 mL) was added TFA (3 mL)) at 0° C. The reaction mixture was allowed to warm up to room temperature and stir for 5 h/16 h. Progress of the reaction was monitored by TLC. After completion of the reaction, solvents was evaporated under reduced pressure, sat. NaHCO₃ (60 mL) was added and the aqueous solution was extracted with ethyl acetate (2×100 mL), dried over anhydrous sodium sulfate, filtered and concentrated under vacuum to obtain Int-3 (1.20 g, 78%) as a pale yellow solid.

¹H NMR (400 MHz, DMSO-d₆) δ 10.01 (br s, 1H), 9.08 (br s, 3H), 8.32 (d, J=9.00 Hz, 1H), 7.80 (d, J=9.39 Hz, 1H), 7.34 (s, 1H), 6.53 (br s, 1H), 5.41 (s, 2H), 5.30 (s, 2H), 4.79 (br s, 2H), 3.91 (br s, 2H), 3.69 (br s, 2H), 3.11 (d, J=12.91 Hz, 1H), 2.84 (br s, 6H), 1.86-1.92 (m, 2H), 1.38 (br s. 6H), 1.21 (d, J=6.65 Hz, 1H), 0.85 (t, J=7.24 Hz, 3H). LCMS: 562.5 [M+H]⁺.

### Step-4: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-(1-(6-(((1r,4r)-4-((3-chloro-4-cyanophenyl)(methyl)-amino)cyclohexyl)carbamoyl)pyridazin-3-yl)piperidine-4-carbonyl)-2,5-dimethylpiperazine-1-carboxylate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-dimethylpiperazine-1-carboxylate (Int-3, 380 mg, 0.67 mmol, 1.2 eq.) and 1-(6-(((1r,4r)-4-((3-chloro-4-cyanophenyl)(methyl)amino)cyclohexyl)-carbamoyl)pyridazin-3-yl)piperidine-4-carboxylic acid (Int-6, 280 mg, 0.56 mmol, 1.0 eq.) in DMF (3 mL) were added HATU (323 mg, 0.85 mmol, 1.5 eq.) and DIPEA (0.20 mL, 1.5 mmol, 2 eq.) at room temperature and stirring was allowed for an additional 16 h. Progress of the reaction was monitored by TLC. After completion of the reaction, water (50 mL) was added and the aqueous solution was extracted with ethyl acetate (2×200 mL). The combined organic extract was washed with water (100 mL), brine (100 mL) and dried over anhydrous sodium sulfate. Solvents were evaporated under reduced pressure to obtain the crude product which was purified by prep. HPLC using ammonium bicarbonate in water and acetonitrile as the mobile phase to afford the title compound (108 mg, 37%) as an off-white solid.

¹H NMR (400 MHz, DMSO-d₆) δ 8.96 (s, 1H), 8.49 (br d, J=8.25 Hz, 1H), 8.13 (br d, J=9.01 Hz, 1H), 7.84 (d, J=9.63 Hz, 1H), 7.55-7.73 (m, 2H), 7.29-7.46 (m, 2H), 6.95 (d, J=2.38 Hz, 1H), 6.83 (dd, J=9.07, 2.31 Hz, 1H), 6.52 (s, 1H), 5.43 (s, 2H), 5.32 (s, 2H), 4.70-4.79 (m, 1H), 4.42-4.64 (m, 3H), 4.16-4.38 (m, 1H), 3.57-4.03 (m, 6H), 3.10-3.14 (m, 3H), 2.85 (s, 3H), 2.21 (d, J=6.82 Hz, 6H), 1.82-1.99 (m, 5H), 1.60-1.81 (m, 9H), 1.42 (br dd, J=10.51, 6.75 Hz, 2H), 1.32 (br d, J=6.75 Hz, 1H), 1.21-1.29 (m, 3H), 1.12 (br dd, J=10.38, 6.88 Hz, 1H), 0.89 (t, J=7.32 Hz, 3H). LCMS: 1041.7 [M+H]⁺. HPLC: 99.42%.

### Example S18. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((1-(6-(((1r,4r)-4-((3-chloro-4-cyanophenyl)(methyl)-amino)cyclohexyl)carbamoyl)pyridazin-3-yl)piperidin-4-yl)methyl)piperazine-1-carboxylate (Compound No. 18)

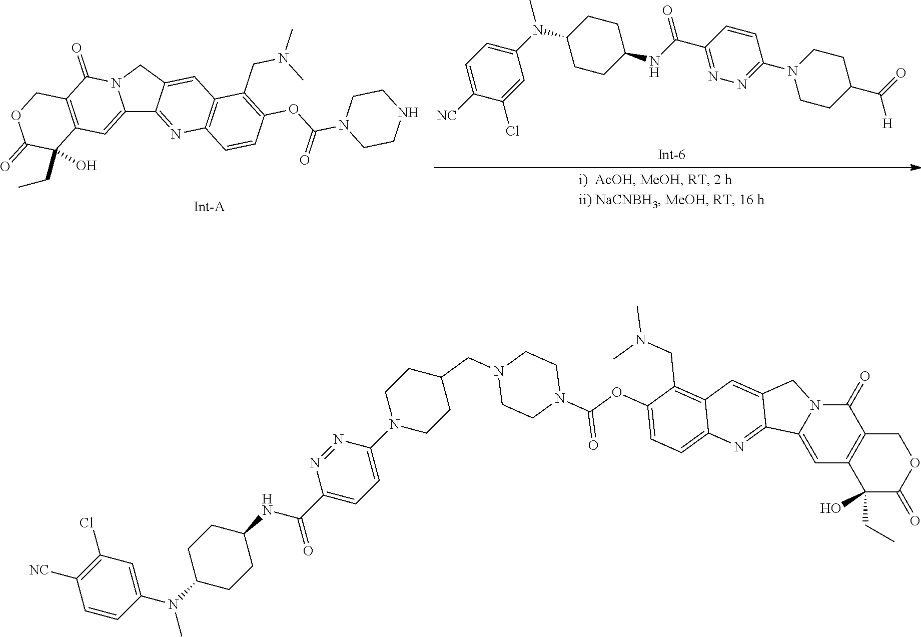 [SMILES: CC[C@@]1(O)C(=O)OCc2c1cc1n(c2=O)Cc2cc3c(CN(C)C)c(OC(=O)N4CCNCC4)ccc3nc2-1 ; CN(c1ccc(C#N)c(Cl)c1)[C@H]1CC[C@H](NC(=O)c2ccc(N3CCC(C=O)CC3)nn2)CC1 ; CC[C@@]1(O)C(=O)OCc2c1cc1n(c2=O)Cc2cc3c(CN(C)C)c(OC(=O)N4CCN(CC5CCN(c6ccc(C(=O)N[C@H]7CC[C@H](N(C)c8ccc(C#N)c(Cl)c8)CC7)nn6)CC5)CC4)ccc3nc2-1]

To a stirred solution of N-((1r,4r)-4-((3-chloro-4-cyanophenyl)(methyl)amino)cyclohexyl)-6-(4-formylpiperidin-1-yl)pyridazine-3-carboxamide (Int-6, 280 mg, 0.58 mmol, 1.0 eq) in methanol (5 mL) were added(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate (Int-A, 312 mg, 0.58 mmol, 1.0 eq) and acetic acid (0.1 mL) at room temperature and stirred for 2 h. To this reaction mixture, NaCNBH₃ (58 mg, 0.9 mmol, 1.5 eq) was added portion wise at 0° C. The reaction mixture was allowed to warm up to room temperature and stir for 16 h. Progress of the reaction was monitored by TLC. After completion of the reaction, cold water (50 mL) was added and extracted with ethyl acetate (2×200 mL). The combined organic extract was washed with water (100 mL), brine (100 mL), dried over anhydrous sodium sulphate, filtered and concentrated under vacuum to obtain the crude product. The crude obtained was purified by combiflash column eluting with 8% methanol in DCM to afford the title compound (125 mg, 23%) as an off-white solid.

¹H NMR (400 MHz, DMSO-d₆) δ 8.95 (s, 1H), 8.51 (br d, J=8.31 Hz, 1H), 8.12 (d, J=8.80 Hz, 1H), 7.82 (d, J=9.29 Hz, 1H), 7.59-7.66 (m, 2H), 7.31-7.38 (m, 2H), 6.95 (br s, 1H), 6.83 (br d, J=9.29 Hz, 1H), 6.54 (s, 1H), 5.44 (s, 2H), 5.32 (br s, 2H), 4.48-4.52 (m, 2H), 3.70-3.81 (m, 5H), 3.47-3.50 (m, 2H), 2.99-3.08 (m, 3H), 2.85 (s, 3H), 2.48-2.51 (m, 4H) 2.20-2.22 (m, 2H), 2.19 (s, 6H) 1.82-1.96 (m, 6H), 1.62-1.79 (m, 5H), 1.11-1.23 (m, 4H), 0.88 (br t, J=6.85 Hz, 3H).

LCMS: 998.2 [M+H]⁺. HPLC Purity: 96.8%.

### Example S19. Preparation of (8S,11R,13S,14S,17R)-17-Acetyl-11-(4-((2-(4-((((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)oxy)methyl)-piperidin-1-yl)-2-oxoethyl)(methyl)amino)phenyl)-13-methyl-3-oxo-2,3,6,7,8,11,12,13,14,15,16,17-dodeca-hydro-1H-cyclopenta[a]phenanthren-17-yl acetate (Compound No. 19)

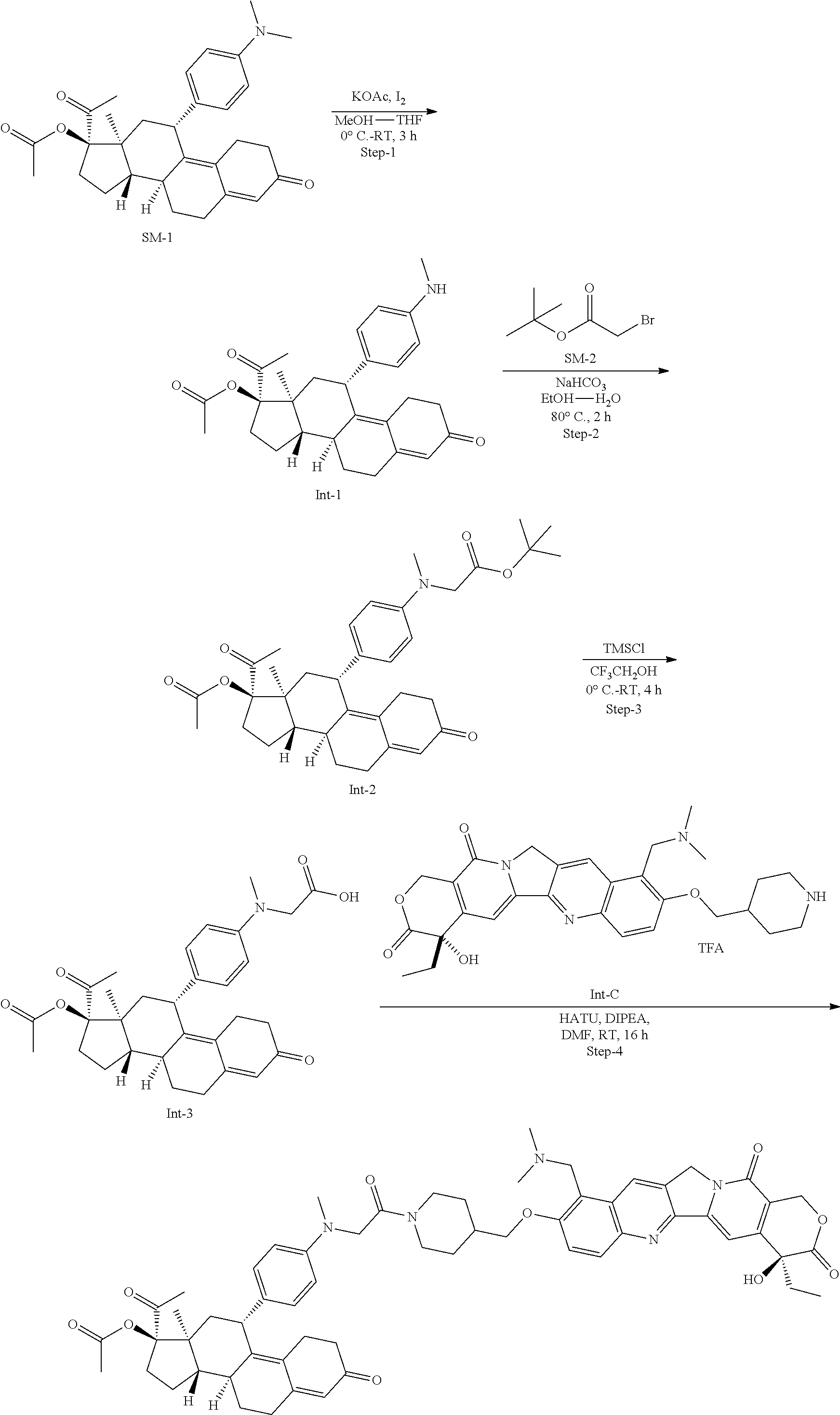

### Step-1: Preparation of (8S,11R,13S,14S,17R)-17-Acetyl-13-methyl-11-(4-(methylamino)phenyl)-3-oxo-2,3,6,7,8,11,12,13,14,15,16,17-dodecahydro-1H-cyclopenta[a]phenanthren-17-yl Acetate (Int-1)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-1, 10 g, 21 mmol, 1.0 eq.) in methanol (150 mL) and THF (150 mL) were added potassium acetate (20.6 g, 210 mmol, 10 eq.) and iodine (13.1 g, 105 mmol, 5 eq.) at 0° C. The reaction mixture was allowed to warm up to room temperature and stir for 3 h. Progress of the reaction was monitored by TLC. After completion of the reaction, the reaction mixture was quenched with sodium thiosulfate (Na₂S₂O₃) solution (50 g in 30 mL water) and extracted with ethyl acetate (2×200 mL). The combined organic extract was washed with brine (100 mL), dried over anhydrous sodium sulfate, filtered and concentrated under vacuum to afford Int-1 (8.0 g, 82%) as an off-white solid which was used in next step without further purification.

¹H NMR (400 MHz, DMSO-d₆) δ 11.91 (br s, 1H), 6.91 (d, J=8.31 Hz, 2H), 6.44 (d, J=8.31 Hz, 2H), 5.67 (s, 1H), 4.37 (m, 1H), 2.75 (s, 2H), 2.61 (d, J=4.40 Hz, 3H), 2.30-2.40 (m, 1H), 2.07-2.16 (s, 5H), 1.99 (s, 6H), 1.63-1.77 (m, 2H), 1.21-1.45 (m, 5H), 0.86 (t, J=6.60 Hz, 1H), 0.16-0.28 (m, 3H). LCMS: 462.28 [M+H]⁺.

### Step-2: Preparation of tert-Butyl N-(4-((8S,11R,13S,14S,17R)-17-Acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycinate (Int-2)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 1 g, 2.17 mmol, 1 eq.) in EtOH:H₂O (20 mL, 1:1) were added SM-2 (0.32 mL, 2.17 mmol, 1 eq.) and NaHCO₃ (911 mg, 10.85 mmol, 5 eq.) at room temperature. The reaction mixture was heated to 80° C. and stirred for 2 h. Progress of the reaction was monitored by TLC. After completion of the reaction, the reaction mixture was diluted with cold water (50 mL) and extracted with ethyl acetate (2×50 mL). The combined organic extract was washed with brine (100 mL), dried over anhydrous sodium sulfate, filtered, and concentrated under vacuum to obtain the crude product. The crude obtained was triturated with n-heptane (2×30 mL), filtered and dried under vacuum to afford Int-2 (900 mg, 72%) as a pale-yellow solid.

¹H NMR (400 MHz, DMSO-d₆) δ 7.00 (d, J=8.31 Hz, 2H), 6.56 (d, J=8.31 Hz, 2H), 5.67 (s. 1H), 5.67 (s, 1H), 4.41 (d, J=6.85 Hz, 1H), 4.00 (s, 2H), 2.91 (s, 3H), 2.58-2.82 (m, 3H), 2.54-2.57 (m, 1H), 2.29-2.38 (m, 1H), 2.06-2.23 (m, 6H), 1.84-2.03 (m, 5H), 1.61-1.79 (m, 2H), 1.34-1.46 (m, 3H), 1.30 (s, 9H), 0.22 (s, 3H). LCMS: 576.2 [M+H]⁺.

### Step-3: Preparation of N-(4-((8S,11R,13S,14S,17R)-17-Acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycine (Int-3)

To a stirred solution of tert-butyl N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycinate (Int-2, 300 mg, 0.521 mmol, 1 eq.) in trifluoroethanol (5 mL) was added chlorotrimethylsilane (1.31 mL, 10.43 mmol, 20 eq.) dropwise at 0° C. The reaction mixture was allowed to warm up to room temperature and stir for 4h. Progress of the reaction was monitored by TLC. After completion of the reaction, solvents were evaporated and the resulting residue was washed with pentane (2×20 mL) and dried under vacuum to afford Int-3 (250 mg, 92%) as a pale-yellow solid.

¹H NMR (400 MHz, DMSO-d₆) δ 6.99 (d, J=8.50 Hz, 2H), 6.55 (d, J=8.76 Hz, 2H), 5.67 (s. 1H), 4.40 (d, J=6.63 Hz, 1H), 4.01 (s, 2H), 2.92 (s, 2H), 2.59-2.79 (m, 3H), 2.55 (br s, 2H), 2.32-2.39 (m, 1H), 2.02-2.22 (m, 1H), 2.13-2.18 (m, 2H), 2.09 (s, 3H), 1.99 (s, 4H), 1.87-1.97 (m, 2H), 1.65-1.75 (m, 2H), 1.33-1.40 (m, 2H), 1.24-1.32 (m, 3H), 0.84-0.87 (m, 2H). LCMS: 520.2 [M+H]⁺.

### Step 4: Preparation of (8S,11R,13S,14S,17R)-17-Acetyl-11-(4-((2-(4-((((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)oxy)methyl)-piperidin-1-yl)-2-oxoethyl)(methyl)amino)phenyl)-13-methyl-3-oxo-2,3,6,7,8,11,12,13,14,15,16,17-dodeca-hydro-1H-cyclopenta[a]phenanthren-17-yl Acetate

To a stirred solution of (S)-10-((dimethylamino)methyl)-4-ethyl-4-hydroxy-9-(piperidin-4-ylmethoxy)-1,12-dihydro-14H-pyrano[3′,4′:6,7]indolizino[1,2-b]quinoline-3,14(4H)-dione TFA salt (Int-C, 350 mg, 0.675 mmol, 1.0 eq.) and N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycine (Int-3, 350 mg, 0.675 mmol, 1.0 eq.) in DMF (4 mL) were added DIPEA (0.35 mL, 2.02 mmol, 3.0 eq.) and HATU (384 mg, 1.01 mmol, 1.5 eq.) at room temperature and stirring was continued for 16 h. Progress of the reaction was monitored by TLC. After completion of the reaction, the reaction mixture was diluted in cold water (30 mL), filtered the resulting solid and purified by prep. HPLC purification eluting with Mobile phase A: 0.1% FA in water and Mobile phase B: Acetonitrile to afford the title compound (24 mg, 3%) as an off-white solid.

¹H NMR (400 MHz, DMSO-d₆) δ 9.34 (br s, 1H), 8.94 (s, 1H), 8.33 (d, J=9.29 Hz, 1H), 7.91 (d, J=9.29 Hz, 1H), 7.30 (s, 1H), 6.93 (d, J=7.83 Hz, 2H), 6.51 (d, J=8.80 Hz, 3H), 5.64 (s, 1H), 5.41 (s, 2H), 5.25 (s, 2H), 4.75 (br s, 2H), 4.36 (br s, 2H), 4.10-4.24 (m, 4H), 3.86-3.97 (m, 2H), 2.88 (br s, 9H), 2.51-2.55 (m, 3H), 2.18-2.59 (m, 2H), 2.07 (s, 8H), 1.96 (s, 9H), 1.60-1.73 (m, 3H), 1.24-1.41 (m, 5H), 0.86 (t, J=6.85 Hz, 3H), 0.20 (s, 2H). LCMS: 1020.5 [M+H]⁺. HPLC purity 94.3%.

### Example S20. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((1-(6-(((1r,4r)-4-((3-chloro-4-cyanophenyl)-(methyl)amino)cyclohexyl)carbamoyl)pyridazin-3-yl)piperidin-4-yl)methyl)-2,5-dimethylpiperazine-1-carboxylate (Compound No. 20)

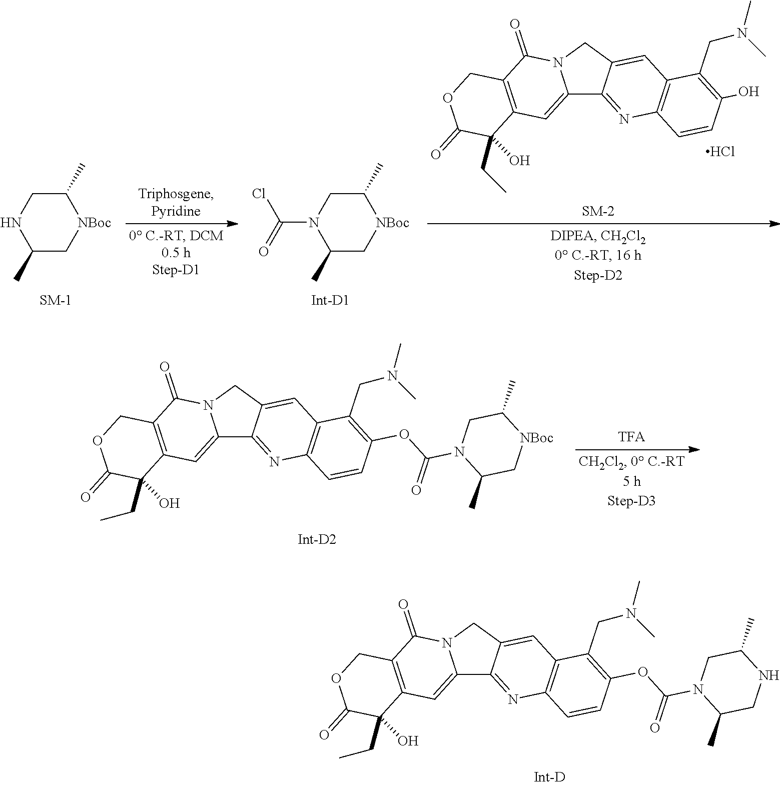

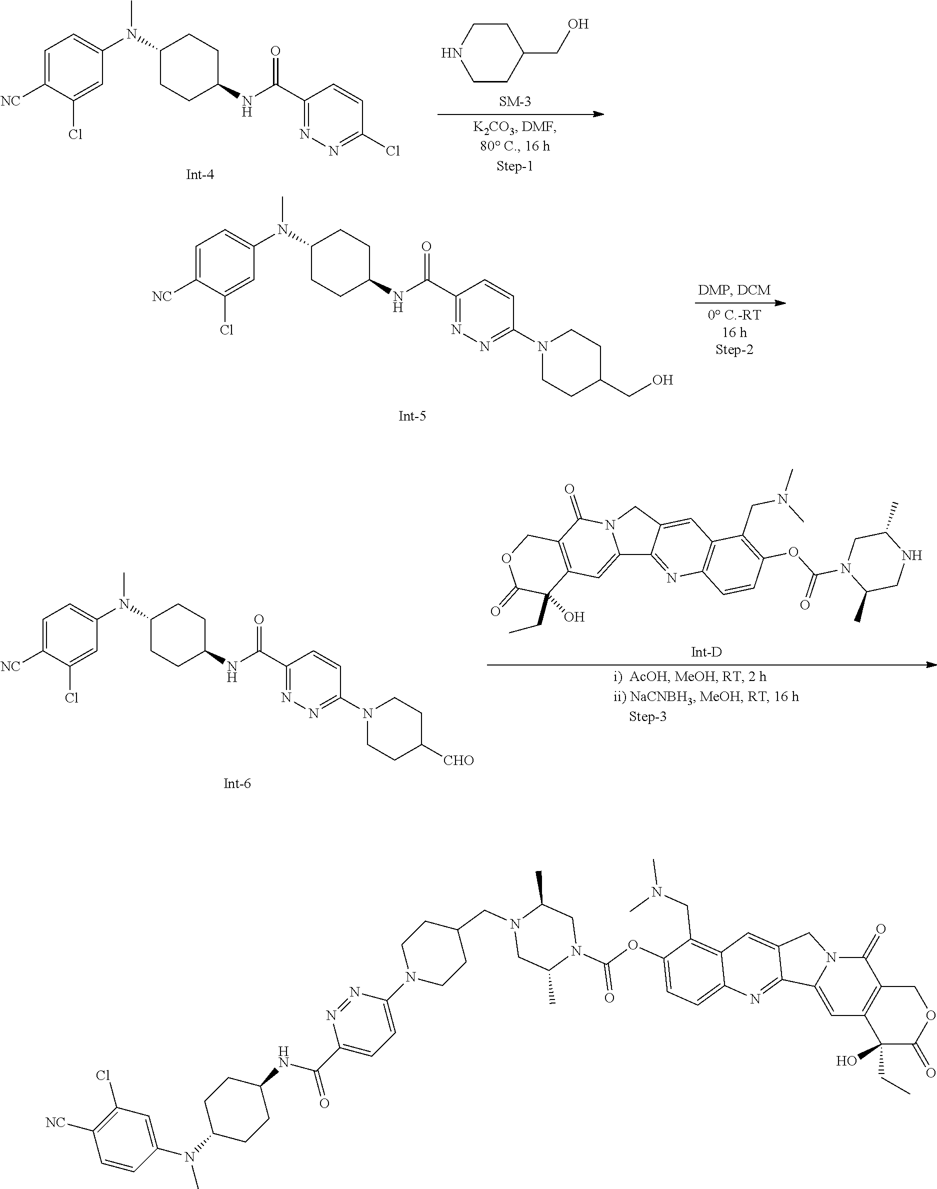

### Step-D1: Preparation of tert-Butyl (2S,5R)-4-(Chlorocarbonyl)-2,5-dimethylpiperazine-1-carboxylate (Int-D1)

To a stirred solution of tert-butyl (2S,5R)-2,5-dimethylpiperazine-1-carboxylate (SM-1, 3.0 g, 13.99 mmol, 1.0 eq.) in DCM (25 mL) were added pyridine (1.7 mL, 20.99 mmol, 1.5 eq.) and triphosgene (1.24 g, 41.99 mmol, 0.3 eq.) solution in DCM (5 mL) dropwise over a period of 10 min at 0° C. The reaction mixture was allowed to warm up to room temperature and stir for 30 min. Progress of the reaction was monitored by TLC (non-polar spot was observed). After completion of the reaction, the reaction mixture was poured into ice cold water (50 mL) and extracted with DCM (2×30 mL). The combined organic extract was washed with brine (100 mL), dried over anhydrous sodium sulfate, filtered and concentrated under vacuum to afford Int-D1 (3.7 g, crude) as a pale brown gum which was used in next step without further purification.

¹H NMR (400 MHz, DMSO-d₆) δ 4.09-4.55 (m, 2H), 3.46-3.69 (m, 2H), 3.13-3.41 (m, 2H), 1.40 (s, 9H), 0.98-1.28 (m, 6H).

### Step-D2: Preparation of 1-(tert-Butyl) 4-((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) (2S,5R)-2,5-dimethyl piperazine-1,4-dicarboxylate (Int-D2)

To a solution of tert-butyl (2S,5R)-4-(chlorocarbonyl)-2,5-dimethylpiperazine-1-carboxylate (Int-D1, 3.6 g, 13.05 mmol, 2 eq.) and (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione hydrochloride (SM-2, 3.1 g, 6.52 mmol, 1 eq.) in THF (30 mL) and DMF (30 mL) was added DIPEA (5.67 mL, 32.6 mmol, 5 eq.) at 0° C. The reaction mixture was allowed to warm up to room temperature and stir for 16 h. Progress of the reaction was monitored by TLC. After completion of the reaction, the reaction mixture was quenched with water (30 mL) and extracted with ethyl acetate (2×25 mL), The combined organic extract was washed with brine (100 mL), dried over anhydrous sodium sulfate, filtered and concentrated under vacuum to obtain the crude product. The crude obtained was purified by combiflash column chromatography eluting with 10% methanol in DCM to afford Int-D2 (2.5 g, 58%) as a pale yellow foam.

¹H NMR (400 MHz, DMSO-d₆) δ 8.95 (br s, 1H), 8.11 (d, J=9.29 Hz, 1H), 7.54-7.76 (m, 1H), 7.34 (s, 1H), 6.53 (s, 1H), 5.43 (s, 2H), 5.31 (s, 2H), 4.19-4.41 (m, 2H), 3.61-3.81 (m, 2H), 3.17 (d, J=12.91 Hz, 1H), 2.20 (d, J=6.36 Hz, 6H), 1.80-1.95 (m, 3H), 1.26-1.41 (m, 14H), 1.18 (d, J=6.36 Hz, 3H), 0.89 (t, J=6.36 Hz, 3H). LCMS: 662.4 [M+H]⁺.

### Step-D3: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-Dimethylpiperazine-1-carboxylate (Int-D)

To a stirred solution of 1-(tert-butyl) 4-((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) (2S,5R)-2,5-dimethylpiperazine-1,4-dicarboxylate (Int-D2, 1.5 g, 2.26 mmol, 1.0 eq.) in DCM (20 mL) was added TFA (3 mL)) at 0° C. The reaction mixture was allowed to war up to room temperature and stir for 5 h. Progress of the reaction was monitored by TLC. After completion of the reaction, solvents were evaporated under reduced pressure, saturated NaHCO₃ solution (50 mL) was added and the aqueous solution was extracted with EtOAc (2×60 mL), dried with sodium sulfate, filtered and concentrated under reduced pressure to afford Int-D (1.20 g, 78%) as a pale-yellow solid.

¹H NMR (400 MHz, DMSO-d₆) δ 10.01 (br s, 1H), 9.08 (br s, 3H), 8.32 (d, J=9.00 Hz, 1H), 7.80 (d, J=9.39 Hz, 1H), 7.34 (s, 1H), 6.53 (br s, 1H), 5.41 (s, 2H), 5.30 (s, 2H), 4.79 (br s, 2H), 3.91 (br s, 2H), 3.69 (br s, 2H), 3.11 (d, J=12.91 Hz, 1H), 2.84 (br s, 6H), 1.86-1.92 (m, 2H), 1.38 (br s, 6H), 1.21 (d, J=6.65 Hz, 1H), 0.85 (t, J=7.24 Hz, 3H). LCMS: 562.5 [M+H]⁺.

### Step-1: Preparation of N-((1r,4r)-4-((3-Chloro-4-cyanophenyl)(methyl)amino)cyclohexyl)-6-(4-(hydroxymethyl)piperidin-1-yl)pyridazine-3-carboxamide (Int-5)

To a stirred solution of Int-4 (2.0 g, 5 mmol, 1.0 eq.) in DMF (10 mL) was added piperidin-4-ylmethanol (SM-3, 0.690 g, 6 mmol, 1.2 eq.) and K₂CO₃ (1.1 g, 8 mmol, 1.6 eq.) at room temperature. The reaction mixture was heated to 90° C. 16 h. Progress of the reaction was monitored by TLC. After completion of the reaction, cold water (50 mL) was added to this reaction mixture and extracted with ethyl acetate (2×150 mL). The combined organic layer extract was washed with water (100 mL), brine (100 mL) and dried over anhydrous sodium sulfate, filtered, and concentrated under vacuum to obtain the crude product. The crude obtained was purified by combiflash column eluting with 100% ethyl acetate to afford Int-5 (1.3 g, 54%) as an off white solid.

¹H NMR (400 MHz, DMSO-d₆) δ 8.49 (br d, J=8.31 Hz, 1H), 7.80 (d, J=9.78 Hz, 1H), 7.61 (d, J=8.80 Hz, 1H), 7.33 (d, J=9.78 Hz, 1H), 6.94 (d, J=1.96 Hz, 1H), 6.78-6.86 (m, 1H), 4.42-4.55 (m, 3H), 3.72-3.92 (m, 2H), 3.20-3.27 (m, 3H), 2.98 (br t, J=12.47 Hz, 2H), 2.85 (s, 3H), 1.91 (br d, J=9.29 Hz, 2H), 1.64-1.78 (m, 8H), 1.08-1.21 (m, 2H). LCMS: 483.2 [M+H]⁺.

### Step-2: Preparation of N-((1r,4r)-4-((3-Chloro-4-cyanophenyl)(methyl)amino)cyclohexyl)-6-(4-formylpiperidin-1-yl)pyridazine-3-carboxamide (Int-6)

To a stirred solution of N-((1r,4r)-4-((3-chloro-4-cyanophenyl)(methyl)amino)cyclohexyl)-6-(4-(hydroxymethyl)piperidin-1-yl)pyridazine-3-carboxamide (Int-5, 350 mg, 0.72 mmol, 1.0 eq.) in DCM (6 mL) under nitrogen atmosphere was added Dess-Martin periodinane (400 mg, 0.92 mmol, 1.3 eq.) portionwise at 0° C. The reaction mixture was allowed to warm up to room temperature and stir for 16 h. Progress of the reaction was monitored by TLC. After completion of the reaction, water (50 mL) was added and the aqueous reaction mixture was extracted with DCM (2×50 mL). The combined organic extract was washed with saturated NaHCO₃ (50 mL) and saturated sodium thiosulfate (50 mL) solution, dried over anhydrous sodium sulfate, filtered and concentrated under reduced pressure to obtain the crude product Int-6 (320 mg, 91%) as an off-white solid which was used in next step without purification.

¹H NMR (400 MHz, DMSO-d₆) δ 9.62 (s, 1H), 8.51 (br d, J=8.31 Hz, 1H), 7.82 (d, J=9.78 Hz, 1H), 7.61 (d, J=8.80 Hz, 1H), 7.29-7.41 (m, 1H), 6.94 (s, 1H), 6.82 (br d, J=7.83 Hz, 1H), 4.31 (br d, J=13.21 Hz, 1H), 3.98-4.08 (m, 1H), 3.72-3.91 (m, 1H), 3.21-3.31 (m, 2H), 2.85 (s, 3H), 2.65-2.68 (m, 1H), 1.86-2.03 (m, 4H), 1.60-1.82 (m, 5H), 1.45-1.60 (m, 2H), 1.17 (t, J=7.09 Hz, 2H). LCMS: 481.2 [M+H]⁺.

### Step-3: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((1-(6-(((1r,4r)-4-((3-chloro-4-cyanophenyl)(methyl)-amino)cyclohexyl)carbamoyl)pyridazin-3-yl)piperidin-4-yl)methyl)-2,5-dimethylpiperazine-1-carboxylate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-dimethylpiperazine-1-carboxylate (Int-6, 320 mg, 0.66 mmol, 1.0 eq.) in methanol (5 mL) were added Int-D (486 mg, 0.86 mmol, 1.3 eq.) and acetic acid (0.1 mL) at room temperature and the reaction mixture was allowed to stir for an additional 2 h. To this reaction mixture was added NaCNBH₃ (64 mg, 1 mmol, 1.5 eq.) portion wise at 0° C. The reaction mixture was allowed to warm up to room temperature and allowed to stir for 16 h. Progress of the reaction was monitored by TLC. After completion of the reaction, cold water (50 mL) was added and extracted with ethyl acetate (2×200 mL). The combined organic extract was washed with water (100 mL), brine (100 mL) and dried over anhydrous sodium sulfate, filtered and concentrated under vacuum to obtain the crude product. The crude obtained was purified by combiflash column eluting with 6% methanol in DCM to afford the title compound (369 mg, 54%) as an off-white solid.

¹H NMR (400 MHz, DMSO-d₆) δ 8.95 (s, 1H), 8.48 (br d, J=8.38 Hz, 1H), 8.11 (d, J=9.13 Hz, 1H), 7.81 (d, J=9.51 Hz, 1H), 7.57-7.65 (m, 2H), 7.31-7.38 (m, 2H), 6.95 (d, J=2.38 Hz, 1H), 6.83 (dd, J=9.13, 2.50 Hz, 1H), 6.51 (s, 1H), 5.28-5.46 (m, 4H), 4.43-4.57 (m, 2H), 3.71-3.89 (m, 5H), 2.96-3.11 (m, 4H), 2.85 (s, 3H), 2.80-2.82 (m, 2H), 2.41 (br d, J=11.63 Hz, 1H), 2.20-2.35 (m, 3H), 2.14 (s, 6H), 1.58-2.03 (m, 14H), 1.30-1.45 (m, 2H), 1.10-1.22 (m, 1H), 0.96-1.12 (m, 3H), 0.89 (t, J=7.32 Hz, 3H). LCMS: 1026.4 [M+H]⁺. HPLC purity: 94.5%.

### Example S21. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-(1-(6-(((1r,4r)-4-(3-chloro-4-cyano-2-methylphenoxy)cyclohexyl)carbamoyl)pyridazin-3-yl)piperidine-4-carbonyl)-2,5-dimethylpiperazine-1-carboxylate Formate (Compound No. 21)

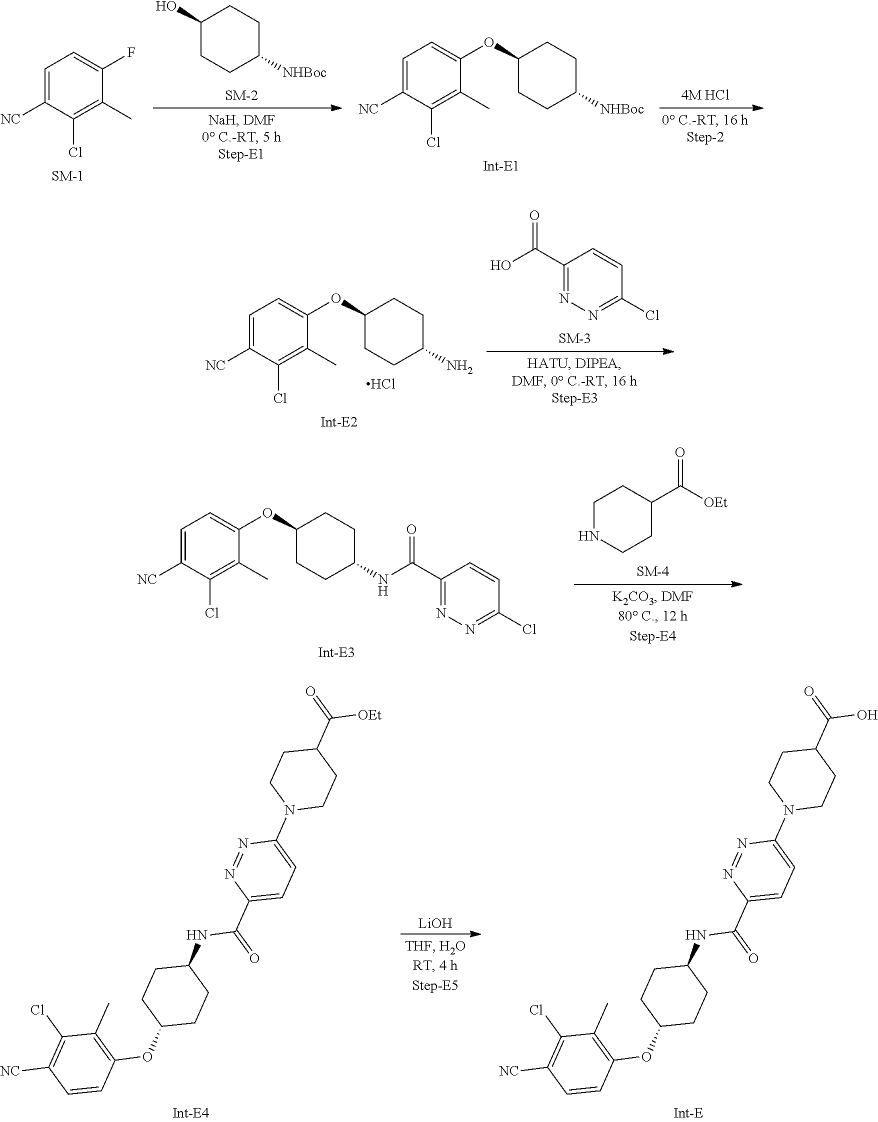

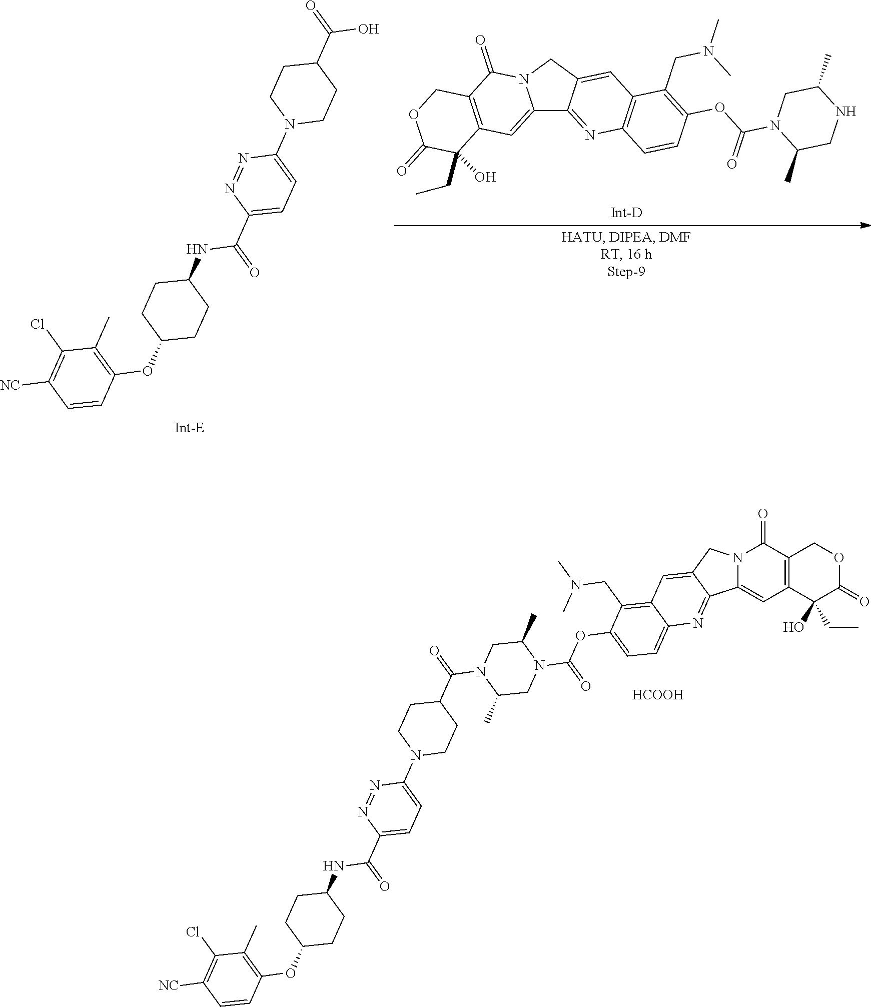

### Step-E1: Preparation of tert-Butyl ((1r,4r)-4-(3-Chloro-4-cyano-2-methylphenoxy)cyclohexyl)carbamate (Int-E1)

To a stirred solution of tert-butyl ((1r,4r)-4-hydroxycyclohexyl)carbamate (SM-2, 10 g, 46.5 mmol, 1.0 eq.) in DMF (100 mL) under nitrogen atmosphere was added NaH (4.08 g, 102 mmol, 2.2 eq.) at 0° C., warmed up to room temperature and stirred for 30 min. To this reaction mixture, 2-chloro-4-fluoro-3-methylbenzonitrile (SM-1, 7.86 g, 46.51 mmol, 1.0 eq.) was added portion wise over a period of 10 min at room temperature and the resulting reaction mixture was allowed to stir for 3 h. Progress of the reaction was monitored by TLC. After completion of the reaction, ice cold water (100 mL) was added and the precipitated solid formed was filtered and dried to afford Int-E1 (15 g, 88%) as an off-white solid.

¹H NMR (400 MHz, DMSO-d₆) δ 7.73 (d, J=8.31 Hz, 1H), 7.22 (d, J=8.80 Hz, 1H), 6.82 (s, 1H), 4.37-4.55 (m, 2H), 2.21 (s, 3H), 1.98-2.11 (m, 2H), 1.76-1.88 (m, 2H), 1.42-1.51 (m, 4H), 1.38 (s, 9H). LCMS: 309 [M−56+H]⁺.

### Step-E2: Preparation of 4-(((1r,4r)-4-Aminocyclohexyl)oxy)-2-chloro-3-methylbenzonitrile Hydrochloride (Int-E2)

To a solution of Int-E1 (15 g, 41.1 mmol) in DCM (25 mL), 4M HCl in 1,4-dioxane (75 mL) was added at 0° C. The reaction mixture was allowed to warm up to room temperature and stir for 16 h. After completion of the reaction, the volatiles were evaporated under reduced pressure and the resulting residue was washed with diethyl ether (2×80 mL) to afford Int-E² (15 g, crude) as an off-white solid which was used as is in the next step.

¹H NMR (400 MHz, DMSO-d₆) δ 8.30 (br s, 3H), 7.75 (d, J=8.80 Hz, 1H), 7.28 (d, J=8.80 Hz, 1H), 4.44-4.55 (m, 1H), 3.00-3.15 (m, 1H), 2.21 (s, 3H), 2.07-2.15 (m, 2H), 1.97-2.06 (m, 2H), 1.41-1.63 (m, 4H). LCMS: 264.75 [M+H]⁺.

### Step-E3: Preparation of 6-Chloro-N-((1r,4r)-4-(3-chloro-4-cyano-2-methylphenoxy)cyclohexyl)pyridazine-3-carboxamide (Int-E3)

To A mixture of 4-(((1r,4r)-4-aminocyclohexyl)oxy)-2-chloro-3-methylbenzonitrile hydrochloride (Int-E2, 15 g, 56.9 mmol, 1.0 eq.) and 6-chloropyridazine-3-carboxylic acid (SM-3, 9 g, 56.9 mmol, 1.0 eq.) in DMF (100 mL) were added HATU (32.4 g, 85.44 mmol, 1.5 eq.) and DIPEA (49 mL, 284 mmol, 5.0 eq.) at 0° C. Reaction mixture was stirred at RT for 16 h. Progress of the reaction was monitored by TLC. After completion of the reaction, volatiles were evaporated under reduced pressure, diluted with water (500 mL) and extracted with ethyl acetate (3×300 mL), The combined organic extract was washed with water (400 mL), brine (400 mL), dried over anhydrous sodium sulfate, filtered, and concentrated under vacuum to give the crude product which was purified by column chromatography eluting with 60-80% ethyl acetate in hexane to afford Int-E³ (6 g, 27%) as an off-white solid.

¹H NMR (400 MHz, DMSO-d₆) δ 9.12 (d, J=8.31 Hz, 1H), 8.22 (d, J=8.80 Hz, 1H), 8.10 (d, J=8.80 Hz, 1H), 7.77 (d, J=8.80 Hz, 1H), 7.27 (d, J=8.80 Hz, 1H), 4.43-4.57 (m, 1H), 3.85-4.01 (m, 1H), 2.23 (s, 3H), 2.08-2.17 (m, 2H), 1.86-1.96 (m, 2H), 1.64-1.77 (m, 2H), 1.49-1.62 (m, 2H). LCMS: 404.9 [M+H]⁺.

### Step-E4: Preparation of Ethyl 1-(6-(((1r,4r)-4-(3-Chloro-4-cyano-2-methylphenoxy)cyclohexyl)carbamoyl)-pyridazin-3-yl)piperidine-4-carboxylate (Int-E4)

To a stirred solution of 6-chloro-N-((1r,4r)-4-(3-chloro-4-cyano-2-methylphenoxy)cyclohexyl)-pyridazine-3-carboxamide (Int-E3, 7 g, 17 mmol, 1.0 eq.) and ethyl piperidine-4-carboxylate (SM-4, 4 g, 25 mmol, 1.5 eq.) in DMF (70 mL) potassium carbonate (5.86 g, 42 mmol, 2.5 eq.) was added at room temperature. The reaction mixture was heated to 80° C. for 12h. Progress of the reaction was monitored by TLC. After completion of starting material, the reaction mixture was diluted with ice cold water (300 mL), stirred for 10 minutes and the precipitated solid was filtered and dried to afford Int-E⁴ (6 g, 66%) as an off white solid.

¹H NMR (400 MHz, DMSO-d₆) δ 8.57 (d, J=7.82 Hz, 1H), 7.80 (d, J=9.29 Hz, 1H), 7.75 (d, J=8.80 Hz, 1H), 7.35 (d, J=9.78 Hz, 1H), 7.24 (d, J=8.80 Hz, 1H), 4.44-4.55 (m, 1H), 4.30-4.41 (m, 2H), 4.00-4.11 (m, 2H), 3.80-3.92 (m, 1H), 3.08-3.23 (m, 2H), 2.64-2.76 (m, 1H), 2.22 (s, 3H), 2.05-2.15 (m, 2H), 1.85-1.97 (m, 4H), 1.44-1.71 (m, 6H), 1.17 (t, J=7.34 Hz, 3H).

LCMS: 526.2 [M+H]⁺.

### Step-E5: Preparation of 1-(6-(((1r,4r)-4-(3-Chloro-4-cyanophenoxy)cyclohexyl)carbamoyl)pyridazin-3-yl)piperidine-4-carboxylic Acid (Int-E)

To a solution of ethyl 1-(6-(((1r,4r)-4-(3-chloro-4-cyano-2-methylphenoxy)cyclohexyl)carbamoyl)-pyridazin-3-yl)piperidine-4-carboxylate (Int-E4, 6.0 g, 11.5 mmol, 1.0 eq.) in THF (20 mL) and water (5 mL) was added LiOH·H₂O (2.49 g, 57.9 mmol, 5.0 eq.) at 0° C. and the reaction mixture was allowed to stir for 5 h. Progress of the reaction was monitored by TLC. After completion of the reaction, volatiles were removed under reduced pressure, diluted with water (80 mL), acidified with 1N aq. HCl to pH 3 and extracted with ethyl acetate (3×80 mL). The combined organic extract was washed with water (200 mL), brine (200 mL), dried over anhydrous sodium sulfate, filtered, and concentrated under vacuum to afford Int-E (5.4 g, 94%) as an off-white solid.

LCMS: 498.37 [M+H]⁺.

### Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-(1-(6-(((1r,4r)-4-(3-chloro-4-cyano-2-methylphenoxy)cyclohexyl)carbamoyl)pyridazin-3-yl)piperidine-4-carbonyl)-2,5-dimethylpiperazine-1-carboxylate Formate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2S,5R)-2,5-dimethylpiperazine-1-carboxylate (Int-D, 1.19 g, 2.012 mmol, 1.0 eq.) and 1-(6-(((1r,4r)-4-(3-chloro-4-cyano-2-methylphenoxy)cyclohexyl)carbamoyl)-pyridazin-3-yl)piperidine-4-carboxylic acid (Int-E, 1 g, 2.012 mmol, 1.0 eq.) in DMF (10 mL) were added HATU (1.14 g, 3.018 mmol, 1.5 eq.) and DIPEA (1.75 mL, 10.06 mmol, 5.0 eq.) at 0° C. and the reaction mixture was stirred at room temperature for 16 h. Progress of the reaction was monitored by TLC. After completion of the reaction, the reaction mixture was diluted with water (60 mL) and extracted with 10% methanol in DCM (2×60 mL). The combined organic extract was washed with water (80 mL), brine (80 mL), dried over anhydrous sodium sulfate, filtered, and concentrated under vacuum to give the crude product which was purified by prep. HPLC method (0.5% aqueous formic acid in CH3CN) in formic acid method to afford the title compound (600 mg, 28%) as off-white solid.

¹H NMR (400 MHz, DMSO-d₆) δ 8.97 (s, 1H), 8.59 (d, J=8.13 Hz, 1H), 8.14 (dd, J=8.94, 2.56 Hz, 1H), 7.81-7.85 (m, 1H), 7.77 (d, J=8.76 Hz, 1H), 7.60-7.72 (m, 1H), 7.38 (dd, J=9.51, 3.38 Hz, 1H), 7.35 (s, 1H), 7.26 (d, J=8.88 Hz, 1H), 6.52 (s, 1H), 5.41-5.46 (m, 2H), 5.28-5.35 (m, 2H), 4.40-4.65 (m, 4H), 3.72-4.04 (m, 6H), 2.98-3.22 (m, 4H), 2.07-2.36 (m, 12H), 1.81-1.98 (m, 3H), 1.38-1.78 (m, 10H), 1.22-1.34 (m, 4H), 1.08-1.16 (m, 2H), 0.89 (t, J=7.25 Hz, 3H). LCMS: 1041.4 [M+H]⁺. HPLC purity 98.6%.

### Example S22. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((1-(6-(((1r,4r)-4-(3-chloro-4-cyano-2-methylphenoxy)-cyclohexyl)carbamoyl)pyridazin-3-yl)piperidin-4-yl)(methyl)amino)hexyl)piperazine-1-carboxylate Formate (Compound No. 22)

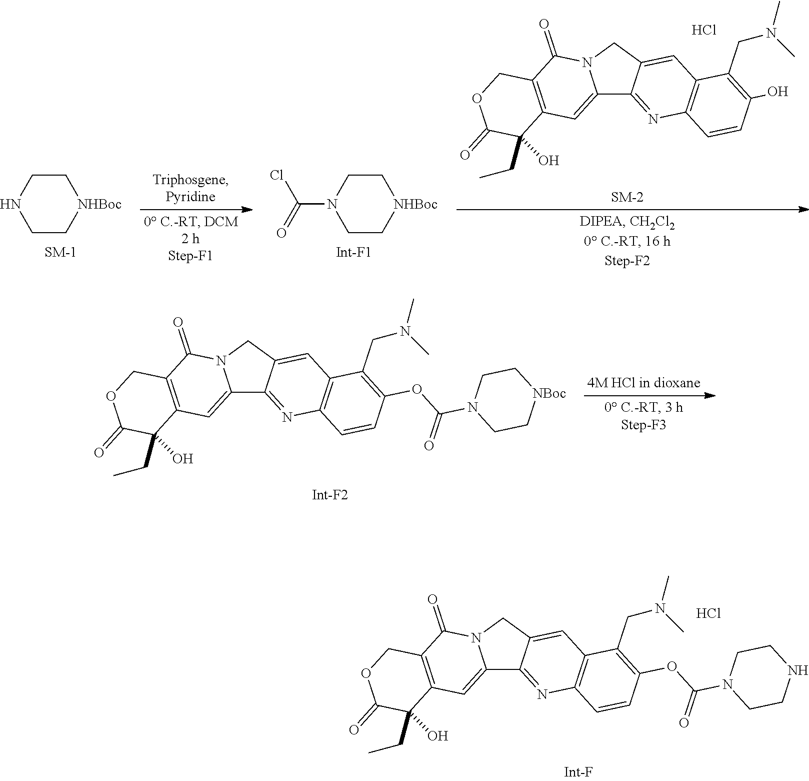

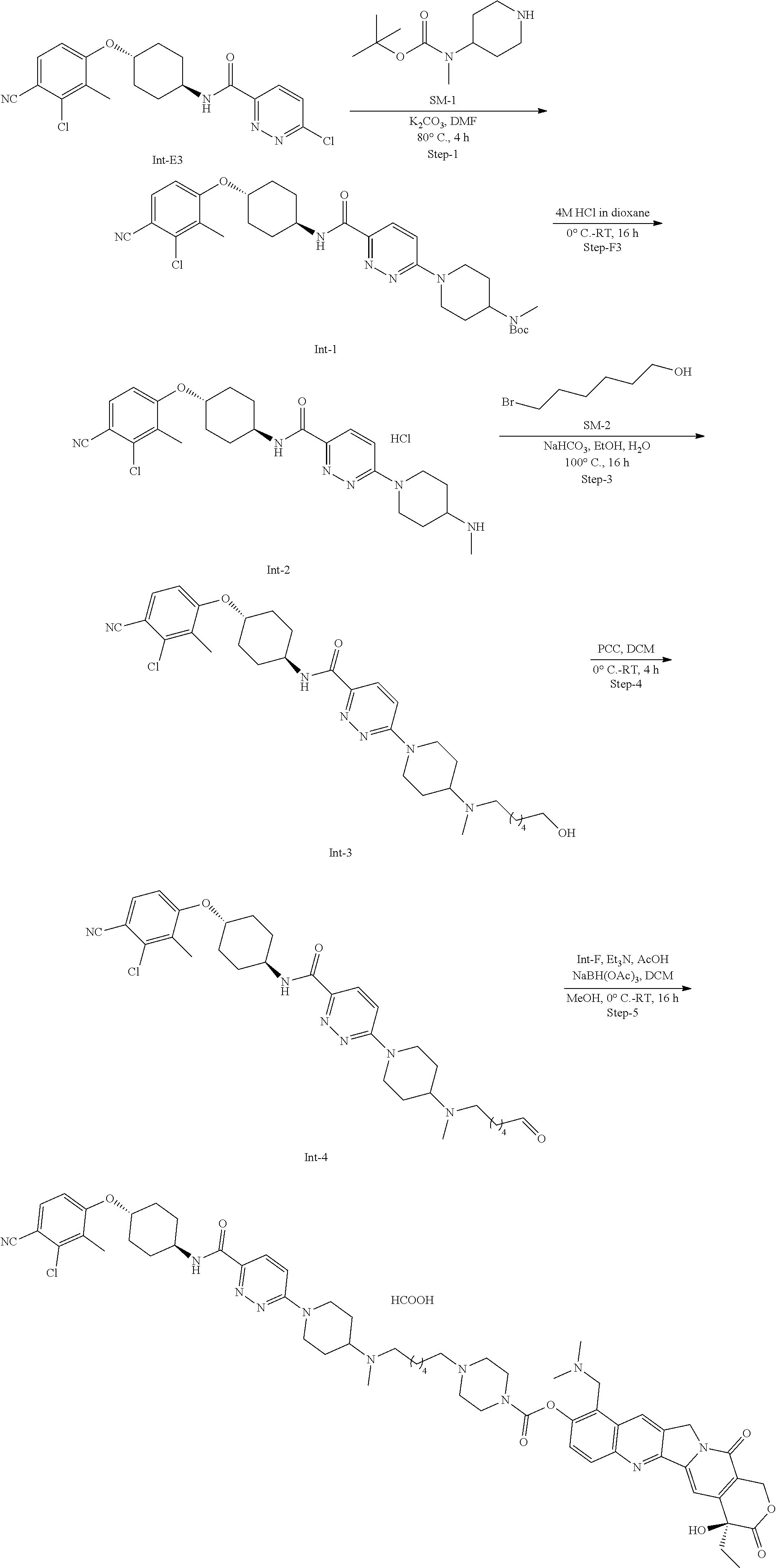

### Step-F1: Preparation of tert-Butyl 4-(Chlorocarbonyl)piperazine-1-carboxylate (Int-F1)

To a stirred solution of tert-butyl piperazine-1-carboxylate (SM-1, 5 g, 26.8 mmol, 1.0 eq.) in DCM (100 mL), pyridine (2.97 g, 37.6 mmol, 1.4 eq.) and triphosgene (3.19 g, 10.7 mmol, 0.4 eq.) were added at 0° C. and allowed to warm up to room temperature and stir for 2 h. Progress of the reaction was monitored by TLC. After completion of the reaction, the mixture was diluted with water (100 mL) and extracted with DCM (2×100 mL). The combined organic extract was again washed with water (200 mL), brine (200 mL), dried over anhydrous sodium sulfate, filtered, and concentrated under vacuum to afford Int-F1 (6.0 g, 90%) as an oil.

¹H NMR (400 MHz, DMSO-d₆) δ 3.64 (d, J=4.40 Hz, 2H), 3.52 (br s, 2H), 3.27-3.47 (m, 3H), 2.93-3.21 (m, 1H), 1.41 (s, 9H).

### Step-F2: Preparation of (S)-1-(tert-Butyl) 4-(10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) Piperazine-1,4-dicarboxylate (Int-F2)

To a stirred solution of (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione hydrochloride (SM-2, 10 g, 23.7 mmol, 1.0 eq.) in DCM (250 mL) were added DIPEA (15.3 g, 118 mmol, 5 eq.) and DMAP (724 mg, 5.9 mmol, 0.25 eq.) followed by addition of tert-butyl 4-(chlorocarbonyl)piperazine-1-carboxylate (Int-F1, 5.89 g, 23.7 mmol, 1 eq.) in DCM (20 mL) dropwise over a period of 10 min at 0° C. and allowed to stir for 16 h at room temperature. Progress of the reaction was monitored by TLC. After completion of the reaction, the mixture was diluted with water (100 mL) and extracted with DCM (3×100 mL). The combined organic extract was again washed with water (200 mL), brine (200 mL), dried over anhydrous sodium sulfate, filtered, and concentrated under vacuum to give the crude product which was purified by combi flash column using 7% methanol in DCM to afford Int-F2 (10 g, 66%) as an off-white solid.

¹H NMR (400 MHz, DMSO-d₆) δ 8.95 (s, 1H), 8.11 (d, J=9.29 Hz, 1H), 7.65 (d, J=9.29 Hz, 1H), 7.34 (s, 1H), 6.53 (s, 1H), 5.43 (s, 2H), 5.31 (s, 2H), 3.67-3.79 (m, 4H), 3.41-3.55 (m, 6H), 2.20 (s, 6H), 1.80-1.93 (m, 2H), 1.44 (s, 9H), 0.89 (t, J=7.34 Hz, 3H). LCMS: 634.2 [M+H]⁺.

### Step-F3: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl Piperazine-1-carboxylate Hydrochloride (Int-F)

To (S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)piperazine-1,4-dicarboxylate (Int-F2, 5 g, 7.89 mmol, 1.0 eq.) under nitrogen atmosphere was added 4M HCl in 1,4-dioxane (50 mL) at 0° C., allowed to warm up to room temperature and stir for 3 h. Progress of the reaction was monitored by TLC. After completion of the reaction, the volatiles were evaporated under reduced pressure and the residue was triturated with diethyl ether (3×100 mL) to afford Int-F (5 g, crude) as an off-white solid.

LCMS: 534.2 [M+H]⁺.

### Step-1: Preparation of tert-Butyl (1-(6-(((1r,4r)-4-(3-chloro-4-cyano-2-methylphenoxy)cyclohexyl)carbamoyl)-pyridazin-3-yl)piperidin-4-yl)(methyl)carbamate (Int-1)

To a stirred solution of 6-chloro-N-((1r,4r)-4-(3-chloro-4-cyano-2-methylphenoxy)cyclohexyl)-pyridazine-3-carboxamide (Int-E3, 6 g, 14.85 mmol, 1.0 eq.) and tert-butyl methyl (piperidin-4-yl)carbamate (SM-1, 4.76 g, 22.27 mmol, 1.5 eq.) in DMF (60 mL) was added potassium carbonate (4.10 g, 29.7 mmol, 2.0 eq.) at room temperature. The reaction mixture was then heated to 80° C. and stirred for 4h. Progress of the reaction was monitored by TLC. After completion of the reaction, the mixture was diluted with ice cold water (300 mL), stirred for 10 minutes and the precipitated solid was filtered and dried to afford Int-1 (6 g, 27%) as an off-white solid.

¹H NMR (400 MHz, DMSO-d₆) δ 8.59 (d, J=8.25 Hz, 1H), 7.82 (d, J=9.63 Hz, 1H), 7.77 (d, J=8.76 Hz, 1H), 7.38 (d, J=9.63 Hz, 1H), 7.26 (d, J=8.88 Hz, 1H), 4.56-4.64 (m, 2H), 4.46-4.55 (m, 1H), 3.97-4.23 (m, 1H), 3.81-3.94 (m, 1H), 2.95-3.07 (m, 2H), 2.64 (s, 3H), 2.24 (s, 3H), 2.08-2.15 (m, 2H), 1.86-1.95 (m, 2H), 1.49-1.71 (m, 8H), 1.39 (s, 9H). LCMS: 583.1 [M+H]⁺.

### Step-2: Preparation of N-((1r,4r)-4-(3-Chloro-4-cyano-2-methylphenoxy)cyclohexyl)-6-(4-(methylamino)-piperidin-1-yl)pyridazine-3-carboxamide Hydrochloride (Int-2)

To a solution of tert-butyl (1-(6-(((1r,4r)-4-(3-chloro-4-cyano-2-methylphenoxy)cyclohexyl)carbamoyl)-pyridazin-3-yl)piperidin-4-yl)(methyl)carbamate (Int-1, 7.0 g, 12.02 mmol, 1.0 eq.) was added 4M HCl in 1,4-dioxane (70 mL) at 0° C. and the mixture was allowed to stir for 16 h at room temperature. After completion of the reaction, the volatiles were evaporated under reduced pressure and the resulting residue was washed with diethyl ether (2×80 mL) to afford Int-2 (7 g, crude) as an off-white solid which was used without further purification.

¹H NMR (400 MHz, DMSO-d₆) δ 9.13 (br s, 2H), 8.61 (d, J=8.31 Hz, 1H), 7.88 (d, J=9.78 Hz, 1H), 7.77 (d, J=8.31 Hz, 1H), 7.47 (d, J=9.78 Hz, 1H), 7.26 (d, J=8.80 Hz, 1H), 4.45-4.56 (m, 2H), 3.83-3.96 (m, 1H), 3.57 (s, 3H), 3.08 (t, J=12.23 Hz, 2H), 2.54 (t, J=5.14 Hz, 2H), 2.24 (s, 3H), 2.06-2.18 (m, 4H), 1.86-1.96 (m, 2H), 1.48-1.72 (m, 6H). LCMS: 483.41 [M+H]⁺.

### Step-3: Preparation of N-((1r,4r)-4-(3-Chloro-4-cyano-2-methylphenoxy)cyclohexyl)-6-(4-((6-hydroxyhexyl)-(methyl)amino)piperidin-1-yl)pyridazine-3-carboxamide (Int-3)

To a solution of N-((1r,4r)-4-(3-chloro-4-cyano-2-methylphenoxy)cyclohexyl)-6-(4-(methylamino)-piperidin-1-yl)pyridazine-3-carboxamide hydrochloride (Int-2, 3.5 g, 7.26 mmol, 1.0 eq.) and 6-bromohexan-1-ol (SM-2, 4.7 mL, 36.3 mmol, 5 eq.) in ethanol (70 mL) and water (70 mL) was added NaHCO₃ (6.1 g, 72.61 mmol, 10 eq.) at room temperature. The reaction mixture was heated to 100° C. and stirred for 16 h. Progress of the reaction was monitored by TLC. After completion of the reaction, the mixture was filtered through a celite pad and washed with ethyl acetate (40 mL). The filtrate was concentrated under reduced pressure, diluted with water (120 mL) and extracted with ethyl acetate (2×200 mL). The combined organic extract was washed with brine (100 mL), dried over anhydrous sodium sulfate, filtered, and concentrated under vacuum to obtain the crude product which was purified by combi flash chromatography eluting with 2-5% methanol in DCM to afford Int-3 (4 g, 47%) as an off-white solid.

¹H NMR (400 MHz, DMSO-d₆) δ 8.59 (d, J=8.31 Hz, 1H), 7.82 (d, J=9.29 Hz, 1H), 7.77 (d, J=8.80 Hz, 1H), 7.37 (d, J=9.29 Hz, 1H), 7.26 (d, J=8.80 Hz, 1H), 4.46-4.62 (m, 3H), 4.29-4.35 (m, 1H), 3.83-3.94 (m, 1H), 3.34-3.41 (m, 3H), 3.00 (t, J=12.23 Hz, 2H), 2.23-2.25 (m, 4H), 2.07-2.16 (m, 3H), 1.74-1.96 (m, 4H), 1.52-1.71 (m, 5H), 1.35-1.51 (m, 6H), 1.20-1.33 (m, 6H). LCMS: 583.17 [M+H]⁺.

### Step-4: Preparation of N-((1r,4r)-4-(3-Chloro-4-cyano-2-methylphenoxy)cyclohexyl)-6-(4-(methyl(6-oxohexyl)amino)piperidin-1-yl)pyridazine-3-carboxamide (Int-4)

To a stirred solution of N-((1r,4r)-4-(3-chloro-4-cyano-2-methylphenoxy)cyclohexyl)-6-(4-((6-hydroxyhexyl)(methyl)amino)piperidin-1-yl)pyridazine-3-carboxamide (Int-3, 4.0 g, 6.87 mmol, 1 eq.) in dichloromethane (50 mL) was added pyridinium chlorochromate (PCC. 2.21 g, 10.30 mmol, 1.5 eq.) portion wise at 0° C. and the reaction mixture was stirred for 4 h at room temperature. Progress of the reaction was monitored by TLC. After completion of the reaction, the reaction mixture was filtered through a celite pad and the filtrate was concentrated under vacuum to afford Int-4 (4.2 g, crude) as a brown solid which was used in the next step as such.

LCMS: 582.2 [M+H]⁺.

### Step-5: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((1-(6-(((1r,4r)-4-(3-chloro-4-cyano-2-methylphenoxy)-cyclohexyl)carbamoyl)pyridazin-3-yl)piperidin-4-yl)(methyl)amino)hexyl)piperazine-1-carboxylate Formate

To a solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate hydrochloride (Int-F, 3.67 g mg, 6.89 mmol, 1 eq.) in methanol and dichloromethane (40, 160 mL) was added Et₃N (0.9 mL, 6.89 mmol, 1.0 eq.) and the resulting solution was allowed to stir for 30 minutes. To this solution. N-((1r,4r)-4-(3-chloro-4-cyano-2-methylphenoxy)cyclohexyl)-6-(4-(methyl(6-oxohexyl)amino)piperidin-1-yl)pyridazine-3-carboxamide (Int-4, 4 g, 6.89 mmol, 1 eq.) and glacial acetic acid (0.1 mL) were added at room temperature and allowed to stir for 2 h. To this reaction mixture was added NaBH(OAc) 3 (5.81 mg, 27.58 mmol, 4 eq.) at 0° C. The reaction mixture was allowed to warm up to room temperature and stir for 16 h. Progress of the reaction was monitored by TLC. After completion of the reaction, the volatiles were evaporated under reduced pressure, quenched with ice cold water (100 mL) and extracted with 10% methanol in DCM (2×80 mL). The combined organic extract was washed with brine (100 mL), dried over anhydrous sodium sulfate, filtered, and concentrated under vacuum to obtain the crude product which was purified by prep. HPLC purification method eluting with Mobile phase A: 0.1% HCOOH in water and Mobile phase B: acetonitrile to afford the title compound (260 mg) as an off-white solid.

¹H NMR (400 MHz, DMSO-d₆) δ 8.94 (s, 1H), 8.57 (d, J=7.83 Hz, 1H), 8.08-8.19 (m, 3H), 7.81 (d, J=9.29 Hz, 1H), 7.76 (d, J=8.31 Hz, 1H), 7.63 (d, J=8.80 Hz, 1H), 7.32-7.40 (m, 2H), 7.21-7.28 (m, 1H), 6.51 (brs, 1H), 5.39-5.45 (m, 2H), 5.27-5.35 (m, 2H), 4.45-4.60 (m, 3H), 3.81-3.94 (m, 2H), 3.61-3.80 (m, 4H), 3.44-3.58 (m, 4H), 2.92-3.06 (m, 3H), 2.72-2.84 (m, 1H), 2.29-2.47 (m, 6H), 2.17-2.26 (m, 10H), 2.06-2.15 (m, 2H), 1.76-1.96 (m, 6H), 1.38-1.69 (m, 10H), 1.23-1.35 (m, 5H), 0.89 (t, J=6.11 Hz, 3H). LCMS: 1098.0 [M+H]⁺. HPLC purity 95.6%.

### Example S23. Preparation of (S)-4-Ethyl-9-methoxy-3,14-dioxo-3,4,12,14-tetrahydro-1H-pyrano[3′,4′:6,7]indolizino[1,2-b]quinolin-4-yl 4-((1-(6-(((1r,4r)-4-(3-chloro-4-cyano-2-methylphenoxy)cyclohexyl)carbamoyl)pyridazin-3-yl)piperidin-4-yl)methyl)piperazine-1-carboxylate (Compound No. 23)

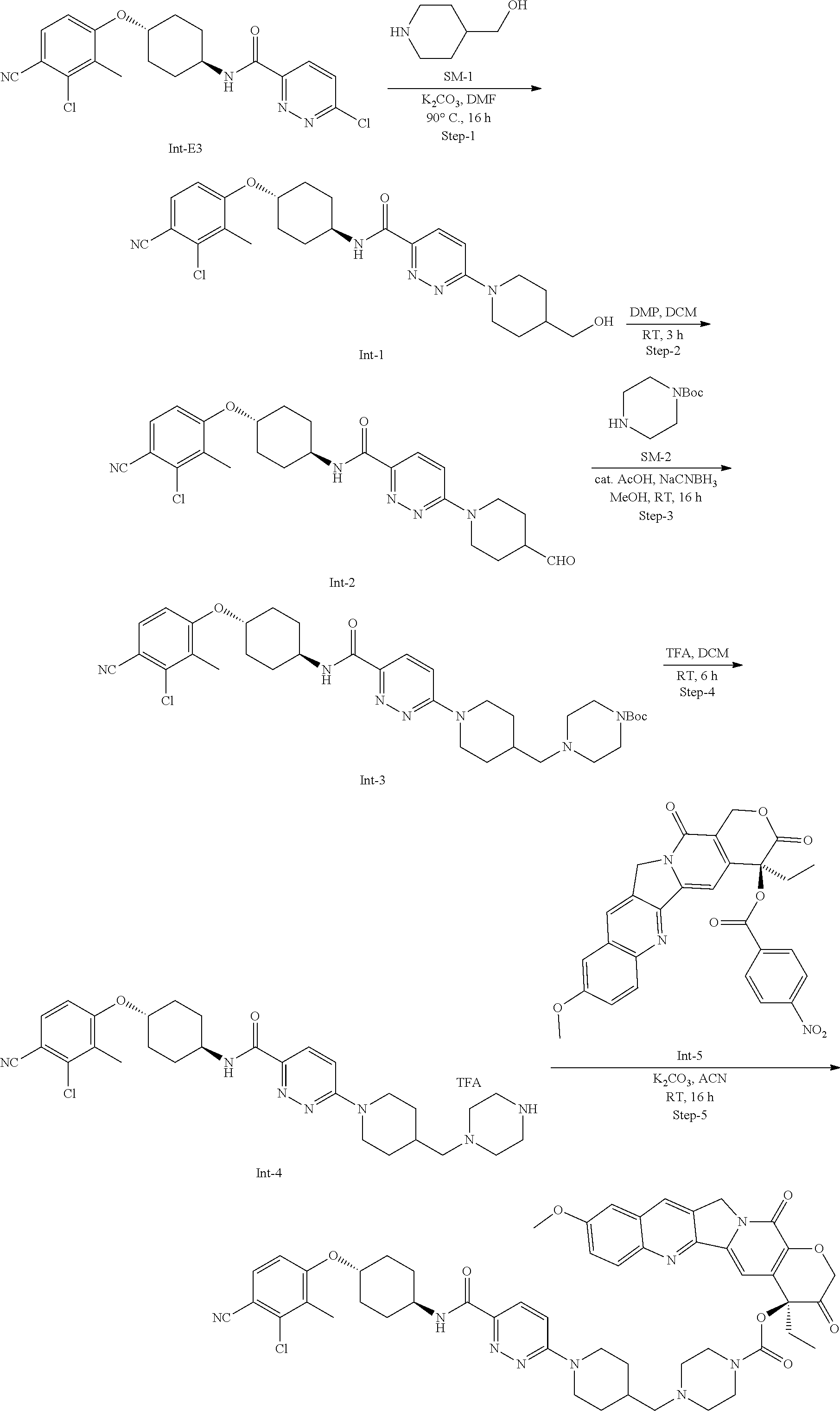

### Step-1: Preparation of N-((1r,4r)-4-(3-Cchloro-4-cyano-2-methylphenoxy)cyclohexyl)-6-(4-(hydroxylmethyl)-piperidin-1-yl)pyridazine-3-carboxamide (Int-1)

A flask was charged with 6-chloro-N-((1r,4r)-4-(3-chloro-4-cyano-2-methylphenoxy)cyclohexyl)-pyridazine-3-carboxamide (Int-E3, 1 g, 2.46 mmol, 1.0 eq.), piperidin-4-ylmethanol (SM-1, 425 mg, 3.70 mmol, 1.5 eq.), K₂CO₃ (1 g, 7.40 mmol, 3.0 eq.) and DMF (10 mL, 10 vol). Reaction mixture was stirred under a nitrogen atmosphere at ambient temperature until TLC indicated complete consumption of starting material. Reaction mixture was diluted with water (100 mL), extracted with ethyl acetate (2×200 mL), combined organic layer washed with brine solution (100 mL) and dried over sodium sulfate. The organic layer was filtered, concentrated under reduced pressure and purified by flash column (silica, 50-60% ethyl acetate/hexane). The pure fractions were combined and concentrated under reduced pressure to obtain N-((1r,4r)-4-(3-chloro-4-cyano-2-methyl-phenoxy)cyclohexyl)-6-(4-(hydroxylmethyl)piperidin-1-yl)pyridazine-3-carboxamide (Int-1, 1 g, 84%) as an off-white solid.

¹H NMR (400 MHz, DMSO-d₆) δ 8.58 (d, J=8.31 Hz, 1H), 7.95 (s, 1H), 7.78 (t, J=9.05 Hz, 2H), 7.33 (d, J=9.78 Hz, 1H), 7.26 (d, J=8.80 Hz, 1H), 4.42-4.55 (m, 5H), 3.81-3.94 (m, 1H), 3.27 (t, J=5.14 Hz, 2H), 2.23 (s, 3H), 2.11 (d, J=10.76 Hz, 2H), 1.90 (d, J=10.27 Hz, 2H), 1.46-1.81 (m, 7H), 1.06-1.22 (m, 2H). LCMS: 484.4 [M+H]⁺.

### Step-2: Preparation of N-((1r,4r)-4-(3-Chloro-4-cyano-2-methylphenoxy)cyclohexyl)-6-(4-formylpiperidin-1-yl)pyridazine-3-carboxamide (Int-2)

A flask was charged with N-((1r,4r)-4-(3-chloro-4-cyano-2-methylphenoxy)cyclohexyl)-6-(4-(hydroxyl-methyl)piperidin-1-yl)pyridazine-3-carboxamide (Int-1, 800 mg, 1.65 mmol, 1.0 eq.), Dess-Martin periodinane (1 g, 2.47 mmol, 1.5 eq.) and DCM (10 mL, 10 vol). Reaction mixture was stirred under a nitrogen atmosphere at ambient temperature until TLC indicated complete consumption of starting material. Reaction mixture was quenched with saturated bicarbonate solution (100 mL) and the desired compound was extracted with DCM (2×200 mL), combined organic layer washed with brine solution (100 mL) and dried over sodium sulfate. The organic solvent was filtered and concentrated under reduced pressure to obtain N-((1r,4r)-4-(3-chloro-4-cyano-2-methyl-phenoxy)cyclohexyl)-6-(4-formylpiperidin-1-yl)pyridazine-3-carboxamide (Int-2, 750 mg, 94%) as an off-white solid, which was used without further purification.

¹H NMR (400 MHz, DMSO-d₆) δ 9.62 (s, 1H), 8.60 (d, J=7.83 Hz, 1H), 7.8 (d, J=8.40 Hz, 1H), 7.37 (d, J=9.78 Hz, 1H), 7.26 (d, J=8.80 Hz, 1H), 4.48-4.50 (m, 1H), 4.31 (d, J=13.21 Hz, 2H), 3.86-3.89 (m, 1H), 3.26 (t, J=11.98 Hz, 2H), 2.61-2.76 (m, 1H), 2.20-2.30 (m, 4H), 2.10-2.12 (m, 2H), 1.81-2.00 (m, 4H), 1.46-1.70 (m, 6H). LCMS: 482.4 [M+H]⁺.

### Step-3: Preparation of tert-Butyl 4-((1-(6-(((1r,4r)-4-(3-Chloro-4-cyano-2-methylphenoxy)cyclohexyl)-carbamoyl)pyridazin-3-yl)piperidin-4-yl)methyl)piperazine-1-carboxylate (Int-3)

To a stirred solution of N-((1r,4r)-4-(3-chloro-4-cyano-2-methylphenoxy)cyclohexyl)-6-(4-formylpiperidin-1-yl)pyridazine-3-carboxamide (Int-2, 1.8 g, 3.74 mmol, 1 eq.) and tert-butyl piperazine-1-carboxylate (SM-2, 1.38 g, 7.46 mmol, 2 eq.) in methanol (18 mL), catalytic amount of glacial acetic acid (0.4 mL) was added at room temperature and stirred for 3 h. To this reaction mixture, NaBH₃CN (462 mg, 7.46 mmol, 2 eq.) was added at 0° C. The reaction mixture was allowed to warm up to room temperature and stir for 16 h. Progress of the reaction was monitored by TLC and LCMS. After completion of the reaction, solvents were evaporated under reduced pressure, quenched with ice cold water (50 mL) and extracted with EtOAc (2×50 mL). The combined organic extract was washed with brine (100 mL), dried over anhydrous sodium sulfate, filtered, and concentrated under vacuum to obtain the crude product. The crude obtained was purified by combiflash column chromatography eluting with 80% EtOAc in heptane to afford Int-3 (1.0 g, 41%) as an off-white solid.

¹H NMR (400 MHz, DMSO-d₆) δ 8.58 (d, J=7.34 Hz, 1H), 7.78 (t, J=7.83 Hz, 2H), 7.32 (d, J=8.31 Hz, 1H), 7.26 (d, J=8.31 Hz, 1H), 4.42-4.57 (m, 3H), 3.81-3.94 (m, 1H), 3.27-3.31 (m, 4H), 2.99 (t, J=12.23 Hz, 2H), 2.26-2.32 (m, 4H), 2.24 (s, 3H), 2.07-2.18 (m, 4H), 1.75-1.96 (m, 5H), 1.48-1.72 (m, 4H), 1.39 (s, 9H), 1.02-1.19 (m, 2H). LCMS: 652.64 [M+H]⁺.

### Step-4: Preparation of N-((1r,4r)-4-(3-Chloro-4-cyano-2-methylphenoxy)cyclohexyl)-6-(4-(piperazin-1-ylmethyl)piperidin-1-yl)pyridazine-3-carboxamide Trifluoroacetate (Int-4)

To a stirred solution of tert-butyl 4-((1-(6-(((1r,4r)-4-(3-chloro-4-cyano-2-methylphenoxy)cyclohexyl)-carbamoyl)pyridazin-3-yl)piperidin-4-yl)methyl)piperazine-1-carboxylate (Int-3, 500 mg, 0.77 mmol, 1.0 eq.) in DCM (10 mL) under nitrogen atmosphere was added TFA (1.17 mL) at 0° C. and the reaction mixture was allowed to stir at room temperature for 16 h. Progress of the reaction was monitored by TLC. After completion of the reaction, solvents were evaporated under reduced pressure to obtain the crude product. The crude obtained was triturated with n-pentane twice (2×30 mL) to afford N-((1r,4r)-4-(3-chloro-4-cyano-2-methylphenoxy)-cyclohexyl)-6-(4-(piperazin-1-ylmethyl)piperidin-1-yl)pyridazine-3-carboxamide trifluoroacetate (Int-4, 348 mg, 82%) as an off-white solid.

LCMS: 552.68 [M+H]⁺.

### Step 5: Preparation of (S)-4-Ethyl-9-methoxy-3,14-dioxo-3,4,12,14-tetrahydro-1H-pyrano[3′,4′:6,7]indolizino-[1,2-b]quinolin-4-yl 4-((1-(6-(((1r,4r)-4-(3-chloro-4-cyano-2-methylphenoxy)cyclohexyl)carbamoyl)pyridazin-3-yl)piperidin-4-yl)methyl)piperazine-1-carboxylate

To a solution of (S)-4-ethyl-9-methoxy-3,14-dioxo-3,4,12,14-tetrahydro-1H-pyrano[3′,4′:6,7]indolizino-[1,2-b]quinolin-4-yl 4-nitrobenzoate (Int-4, 600 mg, 1.1 mmol, 1 eq.) and N-((1r,4r)-4-(3-chloro-4-cyano-2-methylphenoxy)cyclohexyl)-6-(4-(piperazin-1-ylmethyl)piperidin-1-yl)pyridazine-3-carboxamide trifluoroacetate (Int-5, 730 mg, 1.32 mmol, 1.2 eq.) in acetonitrile (10 mL) was added K₂CO₃ (457 mg, 3.31 mmol, 3 eq.). Resulting reaction mixture was allowed to stir at room temperature for 16 h. Progress of the reaction was monitored by LCMS/TLC. After completion of the reaction, the mixture was diluted with water (40 mL) and extracted with ethyl acetate (2×30 mL). Combined organic layer was dried over anhydrous sodium sulfate, filtered and concentrated under reduced pressure to afford the crude product. The crude was purified by prep. HPLC method eluting with Mobile phase A: 0.1% FA in water and Mobile phase B: acetonitrile to afford the title compound (95 mg, 8%) as an off-white solid.

¹H NMR (400 MHz, DMSO-d₆) δ 8.55-8.59 (m, 2H), 8.06 (d, J=9.01 Hz, 1H), 7.80 (d, J=9.63 Hz, 1H), 7.77 (d, J=8.88 Hz, 1H), 7.49-7.55 (m, 2H), 7.33 (d, J=9.76 Hz, 1H), 7.26 (d, J=8.88 Hz, 1H), 7.00 (s, 1H), 5.44 (d, J=3.88 Hz, 2H), 5.29 (s, 2H), 4.42-4.57 (m, 3H), 3.95 (s, 3H), 3.83-3.93 (m, 1H), 3.67-3.78 (m, 1H), 3.55-3.65 (m, 1H), 3.18-3.26 (m, 2H), 2.94-3.06 (m, 2H), 2.53-2.58 (m, 1H), 2.29-2.37 (m, 1H), 2.24 (s, 3H), 2.07-2.22 (m, 8H), 1.86-1.96 (m, 3H), 1.75-1.85 (m, 2H), 1.48-1.71 (m, 4H), 1.05-1.19 (m, 2H), 0.91 (t, J=7.44 Hz, 3H).

LCMS: 956.2 [M+H]⁺. HPLC purity 95.7%.

### Example S24. Preparation of (S)-4-Ethyl-9-methoxy-3,14-dioxo-3,4,12,14-tetrahydro-1H-pyrano[3′,4′:6,7]indolizino[1,2-b]quinolin-4-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate Formate (Compound No. 24)

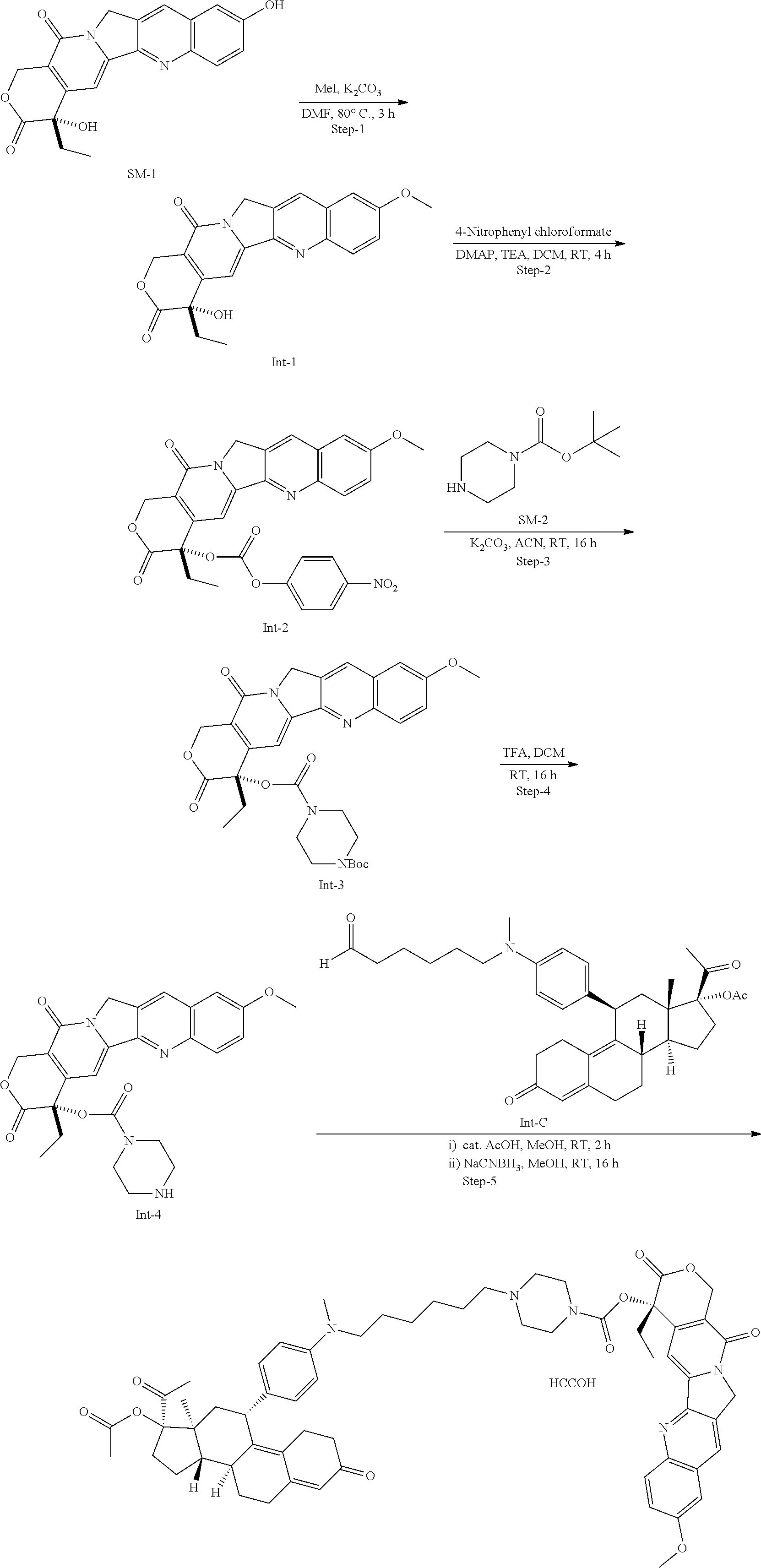

### Step-1: Preparation of (S)-4-Ethyl-4-hydroxy-9-methoxy-1,12-dihydro-14H-pyrano[3′,4′:6,7]indolizino[1,2-b]quinoline-3,14(4H)-dione (Int-1)

A stirred solution of 10-hydroxycamptothecin (SM-1, 1 g, 2.75 mmol, 1 eq.) in DMF (10 mL) was added K₂CO₃ (752 mg, 5.49 mmol, 2 eq.) followed by addition of methyl iodide (0.25 mL, 4.12 mmol, 1.5 eq.) at 0° C. The reaction mixture was allowed to stir at 80° C. for 3 h. Progress of the reaction was monitored by TLC/LCMS. After completion of the reaction, the mixture was poured into ice cold water (30 mL) and the solid obtained was filtered, washed with cold water and dried to afford(S)-4-ethyl-4-hydroxy-9-methoxy-1,12-dihydro-14H-pyrano[3′,4′:6,7]indolizino[1,2-b]quinoline-3,14(4H)-dione (Int-1, 0.93 g, 90%) as an off-white solid.

¹H NMR (400 MHz, DMSO-d₆) δ 8.54 (s, 1H), 8.06 (d, J=9.78 Hz, 1H), 7.47-7.53 (m, 2H), 7.28 (s, 1H), 6.49 (s, 1H), 5.41 (s, 2H), 5.25 (s, 2H), 3.94 (s, 3H), 1.78-1.95 (m, 2H), 0.88 (t, J=7.34 Hz, 3H). LCMS: 379.36 [M+H]⁺.

### Step-2: Preparation of (S)-4-Ethyl-9-methoxy-3,14-dioxo-3,4,12,14-tetrahydro-1H-pyrano[3′,4′:6,7]indolizino-[1,2-b]quinolin-4-yl (4-nitrophenyl) Carbonate (Int-2)

To a stirred solution of (S)-4-ethyl-4-hydroxy-9-methoxy-1,12-dihydro-14H-pyrano[3′,4′:6,7]indolizino-[1,2-b]quinoline-3,14(4H)-dione (Int-1, 1.5 g, 3.96 mmol, 1 eq.) in DCM (15 mL) was added 4-nitrophenyl chloroformate (1.19 g, 5.9 mmol, 1.5 eq.), DMAP (98 mg, 0.79 mmol, 0.2 eq.) and triethyl amine (1.10 mL, 7.9 mmol, 2 eq.) at 0° C. The reaction mixture was allowed to stir at room temperature for 4h. Progress of the reaction was monitored by TLC and LCMS. After completion of the reaction, the mixture was quenched with ice cold water (20 mL) and extracted with 10% MeOH in DCM (2×50 mL). The combined organic layer was dried over anhydrous sodium sulfate and concentrated under reduced pressure. The crude obtained was triturated with diethyl ether twice (2×20 mL) to afford(S)-4-ethyl-9-methoxy-3,14-dioxo-3,4,12,14-tetrahydro-1H-pyrano[3′,4′:6,7]-indolizino[1,2-b]quinolin-4-yl (4-nitrophenyl)carbonate (Int-2, 2.0 g, crude) as a light yellow solid.

¹H NMR (400 MHz, DMSO-d₆) δ 8.49 (d, J=6.85 Hz, 1H), 8.27 (t, J=8.80 Hz, 1H), 8.08 (d, J=8.80 Hz, 2H), 8.01-8.04 (m, 1H), 7.44-7.50 (m, 2H), 6.86 (d, J=8.80 Hz, 2H), 5.38-5.51 (m, 2H), 5.18-5.26 (m, 2H), 3.92 (s, 3H), 2.10-2.32 (m, 2H), 0.85-0.95 (m, 3H).

### Step-3: Preparation of (S)-1-(tert-Butyl) 4-(4-Ethyl-9-methoxy-3,14-dioxo-3,4,12,14-tetrahydro-1H-pyrano[3′,4′:6,7]indolizino[1,2-b]quinolin-4-yl) Piperazine-1,4-dicarboxylate (Int-3)

To a stirred solution of (S)-4-ethyl-9-methoxy-3,14-dioxo-3,4,12,14-tetrahydro-1H-pyrano[3′,4′:6,7]-indolizino[1,2-b]quinolin-4-yl (4-nitrophenyl)carbonate (Int-2, 200 mg, 0.36 mmol, 1 eq.) in acetonitrile (5 mL) was added tert-butyl piperazine-1-carboxylate (82 mg, 0.44 mmol, 1.2 eq.) and K₂CO₃ (149 mg, 1.08 mmol, 3 eq.). The reaction mixture was allowed to stir at room temperature for 16 h. Progress of the reaction was monitored by TLC and LCMS. After completion of the reaction, the mixture was diluted with water (20 mL) and extracted with 5% MeOH in DCM (2×20 mL). The combined organic layer was dried over anhydrous sodium sulfate and concentrated under reduced pressure. The crude obtained was purified by combiflash column chromatography eluting with 100% ethyl acetate to provide(S)-1-(tert-butyl) 4-(4-ethyl-9-methoxy-3,14-dioxo-3,4,12,14-tetrahydro-1H-pyrano[3′,4′:6,7]indolizino[1,2-b]quinolin-4-yl) piperazine-1,4-dicarboxylate (Int-3, 110 mg, 50%) as an off-white solid.

¹H NMR (400 MHz, DMSO-d₆) δ 8.55 (s, 1H), 8.05 (d, J=9.29 Hz, 1H), 7.46-7.56 (m, 2H), 7.01 (s, 1H), 5.42-5.47 (m, 2H), 5.28 (s, 2H), 3.94 (s, 3H), 3.58-3.74 (m, 2H), 3.37-3.55 (m, 2H), 3.21-3.28 (m, 4H), 2.10-2.20 (m, 2H), 1.41 (s, 9H), 0.91 (t, J=7.34 Hz, 3H). LCMS: 591.58 [M+H]⁺.

### Step-4: Preparation of (S)-4-Ethyl-9-methoxy-3,14-dioxo-3,4,12,14-tetrahydro-1H-pyrano[3′,4′:6,7]indolizino-[1,2-b]quinolin-4-yl Piperazine-1-carboxylate (Int-4)

To a stirred solution of (S)-1-(tert-butyl) 4-(4-ethyl-9-methoxy-3,14-dioxo-3,4,12,14-tetrahydro-1H-pyrano[3′,4′:6,7]indolizino[1,2-b]quinolin-4-yl)piperazine-1,4-dicarboxylate (Int-3, 480 mg, 0.81 mmol, 1.0 eq.) in DCM (5 mL) under nitrogen atmosphere was added TFA (1.6 mL) at 0° C. and the reaction mixture was allowed to stir at room temperature for 16 h. Progress of the reaction was monitored by TLC. After completion of the reaction, solvents were evaporated under reduced pressure, sat. NaHCO₃ solution (20 mL) added and the aqueous mixture was extracted with DCM (2×30 mL). The combined organic layer was dried over anhydrous Na₂SO₄, filtered and concentrated under reduced pressure. The crude obtained was triturated with n-pentane twice (2×30 mL) to afford(S)-4-ethyl-9-methoxy-3,14-dioxo-3,4,12,14-tetrahydro-1H-pyrano[3′,4′:6,7]indolizino[1,2-b]quinolin-4-yl piperazine-1-carboxylate (Int-4, 380 mg, 95%) as an off-white solid.

¹H NMR (400 MHz, DMSO-d₆) δ 8.54 (s, 1H), 8.06 (d, J=8.80 Hz, 1H), 7.46-7.54 (m, 2H), 6.97 (s, 1H), 5.36-5.48 (m, 2H), 5.26 (s, 2H), 3.93 (s, 3H), 3.50-3.63 (m, 2H), 3.09-3.22 (m, 2H), 2.70-2.89 (m, 2H), 2.53-2.66 (m, 2H), 2.07-2.16 (m, 2H), 0.88 (t, J=7.09 Hz, 3H). Exchangeable protons were not observed. LCMS: 491.49 [M+H]⁺.

### Step-5: Preparation of (S)-4-Ethyl-9-methoxy-3,14-dioxo-3,4,12,14-tetrahydro-1H-pyrano[3′,4′:6,7]indolizino-[1,2-b]quinolin-4-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate Formate

A stirred solution of (8S,11R,13S,14S,17R)-17-acetyl-13-methyl-11-(4-(methyl(6-oxohexyl)amino)-phenyl)-3-oxo-2,3,6,7,8,11,12,13,14,15,16,17-dodecahydro-1H-cyclopenta[a]phenanthren-17-yl acetate (Int-C, 855 mg, 1.53 mmol, 1.5 eq.) and (S)-4-ethyl-9-methoxy-3,14-dioxo-3,4,12,14-tetrahydro-1H-pyrano[3′,4′:6,7]-indolizino[1,2-b]quinolin-4-yl piperazine-1-carboxylate (Int-4, 500 mg, 1.02 mmol, 1 eq.) in methanol (10 mL) was added triethyl amine (0.14 mL) and glacial acetic acid (0.06 mL) at room temperature and the mixture was allowed to stir for 2 h. To this reaction mixture was added NaCNBH₃ (126 mg, 2.04 mmol, 2 eq.) at 0° C. and allowed to warm up to room temperature and stir for 16 h. Progress of the reaction was monitored by TLC. After completion of the reaction, solvents were evaporated under reduced pressure, quenched with ice cold water (20 mL) and extracted with 10% methanol in DCM (2×20 mL). The combined organic extract was washed with brine (100 mL), dried over anhydrous sodium sulfate, filtered and concentrated under vacuum. The crude obtained was purified by prep. HPLC purification method eluting with Mobile phase A: 0.1% FA in water and Mobile phase B: acetonitrile to afford the title compound (90 mg, 8%) as an off-white solid.

¹H NMR (400 MHz, DMSO-d₆) δ 8.56 (s, 1H), 8.04 (d, J=9.13 Hz, 1H), 7.44-7.56 (m, 2H), 6.93-7.02 (m, 3H), 6.58 (d, J=8.76 Hz, 2H), 5.66 (s, 1H), 5.37-5.49 (m, 2H), 5.28 (s, 2H), 4.39 (d, J=6.75 Hz, 1H), 3.94 (s, 3H), 3.49-3.81 (m, 2H), 3.16-3.25 (m, 4H), 2.81 (s, 3H), 2.64-2.76 (m, 2H), 2.52-2.61 (m, 4H), 2.42-2.47 (m, 2H), 2.21-2.38 (m, 5H), 2.11-2.19 (m, 5H), 2.09 (s, 3H), 1.99 (s, 2H), 1.85-1.95 (m, 2H), 1.61-1.77 (m, 3H), 1.18-1.53 (m, 12H), 0.91 (t, J=7.32 Hz, 3H), 0.23 (s, 3H). LCMS: 1034.4 [M+H]⁺. HPLC purity 93.8%.

### Example S25. Preparation of N-((1r,4r)-4-(3-Chloro-4-cyano-2-methylphenoxy)cyclohexyl)-6-(4-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carbonyl)piperidin-1-yl)pyridazine-3-carboxamide (Compound No. 25)

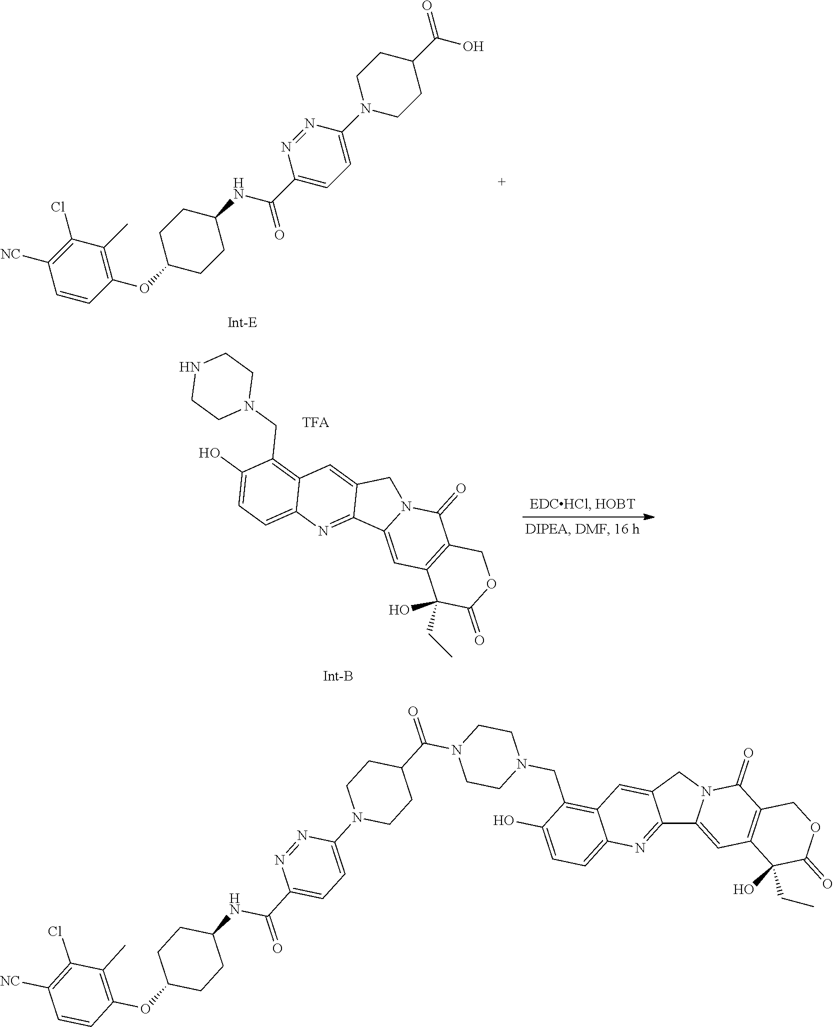 [SMILES: Cc1c(O[C@H]2CC[C@H](NC(=O)c3ccc(N4CCC(C(=O)O)CC4)nn3)CC2)ccc(C#N)c1Cl ; CC[C@@]1(O)C(=O)OCc2c1cc1n(c2=O)Cc2cc3c(CN4CCNCC4)c(O)ccc3nc2-1 ; CC[C@@]1(O)C(=O)OCc2c1cc1n(c2=O)Cc2cc3c(CN4CCN(C(=O)C5CCN(c6ccc(C(=O)N[C@H]7CC[C@H](Oc8ccc(C#N)c(Cl)c8C)CC7)nn6)CC5)CC4)c(O)ccc3nc2-1]

To a stirred solution of 1-(6-(((1r,4r)-4-(3-chloro-4-cyano-2-methylphenoxy)cyclohexyl)carbamoyl)-pyridazin-3-yl)piperidine-4-carboxylic acid (Int-E, 4 g, 8.26 mmol, 1 eq.) in DMF (40 mL) was added EDC·HCl (2.37 g, 12.39 mmol, 1.5 eq.), HOBT (1.89 g, 12.39 mmol, 1.5 eq.) and DIPEA (3.19 g, 24.78 mmol, 3.0 eq.). (S)-4-Ethyl-4,9-dihydroxy-10-(piperazin-1-ylmethyl)-1,12-dihydro-14H-pyrano[3′,4′:6,7]indolizino[1,2-b]quinoline-3,14(4H)-dione trifluoroacetate (Int-B, 5.7 g, 9.91 mmol, 1.2 eq.) was added at 0° C. and the reaction mixture was allowed to stir at RT for 16 h. Progress of the reaction was monitored by TLC. After completion of the reaction, the reaction mixture was diluted with ice cold water (120 mL) and the solid formed was filtered and dried to provide 7.6 g of the crude product as a pale-yellow solid. Some of the crude (2 g) was purified by prep. HPLC method eluting with Mobile phase A: 0.1% FA in water and Mobile phase B: acetonitrile to provide the title compound (508 mg) as a pale-yellow solid.

¹H NMR (400 MHz, DMSO-d₆) δ 8.79 (s, 1H), 8.59 (d, J=7.82 Hz, 1H), 8.00 (d, J=9.29 Hz, 1H), 7.74-7.85 (m, 2H), 7.48 (d, J=8.80 Hz, 1H), 7.36 (d, J=9.29 Hz, 1H), 7.24-7.29 (m, 2H), 6.49 (s, 1H), 5.37-5.45 (m, 2H), 5.23-5.30 (m, 2H), 4.43-4.57 (m, 3H), 4.04 (s, 2H), 3.83-3.95 (m, 1H), 3.43-3.63 (m, 5H), 2.97-3.18 (m, 4H), 2.54-2.64 (m, 2H), 2.24 (s, 3H), 2.07-2.17 (m, 2H), 1.80-1.96 (m, 4H), 1.48-1.78 (m, 9H), 0.88 (t, J=7.09 Hz, 3H). LCMS: 942.3 [M+H]⁺. HPLC purity 96.5%.

### Example S26. Preparation of N-((1r,4r)-4-(3-Chloro-4-cyano-2-methylphenoxy)cyclohexyl)-6-(4-((4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)methyl)piperidin-1-yl)pyridazine-3-carboxamide Formate (Compound No. 26)

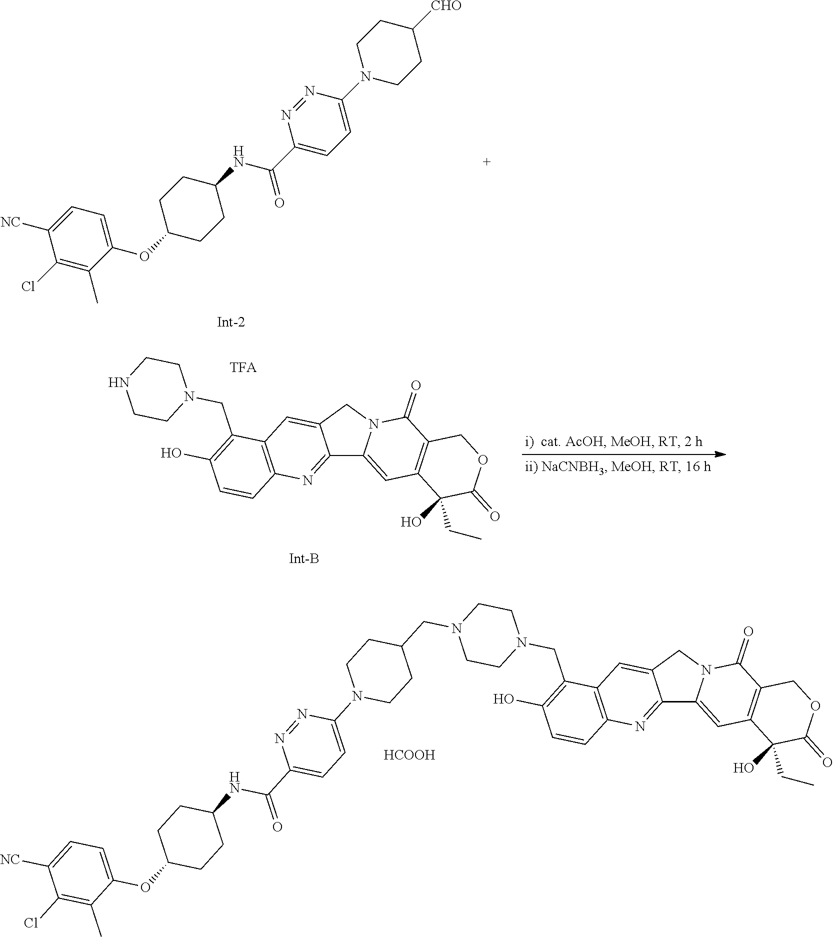 [SMILES: Cc1c(O[C@H]2CC[C@H](NC(=O)c3ccc(N4CCC(C=O)CC4)nn3)CC2)ccc(C#N)c1Cl ; CC[C@@]1(O)C(=O)OCc2c1cc1n(c2=O)Cc2cc3c(CN4CCNCC4)c(O)ccc3nc2-1 ; CC[C@@]1(O)C(=O)OCc2c1cc1n(c2=O)Cc2cc3c(CN4CCN(CC5CCN(c6ccc(C(=O)N[C@H]7CC[C@H](Oc8ccc(C#N)c(Cl)c8C)CC7)nn6)CC5)CC4)c(O)ccc3nc2-1 ; "HCOOH"]

To a stirred solution of N-((1r,4r)-4-(3-chloro-4-cyano-2-methylphenoxy)cyclohexyl)-6-(4-formylpiperidin-1-yl)pyridazine-3-carboxamide (Int-2 of Example S23, 300 mg, 0.62 mmol, 1.2 eq.) and (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione trifluoroacetate (Int-B, 300 mg, 0.52 mmol, 1 eq.) in methanol (6 mL), triethylamine (0.07 mL, 0.52 mmol, 1 eq.) and glacial acetic acid (0.03 mL, 0.52 mmol, 1 eq.) were added at room temperature and the resulting reaction mixture was allowed to stir for 2 h. To this reaction mixture was added NaCNBH₃ (64.5 mg, 1.04 mmol, 2 eq.) at 0° C. The reaction mixture was allowed to warm up to room temperature and stir for 16 h. Progress of the reaction was monitored by TLC. After completion of the reaction, solvents were evaporated under reduced pressure, quenched with ice cold water (20 mL) and extracted with 30% methanol in DCM (2×15 mL). The combined organic extract was washed with brine (100 mL), dried over anhydrous sodium sulfate, filtered and concentrated under vacuum to obtain the crude product. The crude obtained was purified by prep. HPLC purification method eluting with Mobile phase A: 0.1% FA in water and Mobile phase B: acetonitrile to afford the title compound (100 mg, 20%) as a yellow solid.

¹H NMR (400 MHz, DMSO-d₆) δ 8.73 (s, 1H), 8.56 (d, J=8.25 Hz, 1H), 8.21 (s, 1H), 7.98 (d, J=9.13 Hz, 1H), 7.78 (t, J=9.63 Hz, 2H), 7.42 (d, J=9.13 Hz, 1H), 7.32 (d, J=9.76 Hz, 1H), 7.23-7.28 (m, 2H), 6.48 (s, 1H), 5.41 (s, 2H), 5.22-5.30 (m, 2H), 4.41-4.57 (m, 3H), 4.11 (s, 2H), 3.82-3.94 (m, 2H), 2.93-3.06 (m, 4H), 2.55-2.69 (m, 3H), 2.34-2.45 (m, 3H), 2.24 (s, 3H), 2.06-2.20 (m, 5H), 1.76-1.96 (m, 7H), 1.47-1.73 (m, 4H), 1.02-1.17 (m, 2H), 0.88 (t, J=7.19 Hz, 3H). LCMS: 928.0 [M+H]⁺. HPLC purity 95.0%.

### Example S27. Preparation of (S)-10-((4-(1-(6-(((1r,4r)-4-(3-Chloro-4-cyano-2-methylphenoxy)cyclohexyl)carbamoyl)-pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl 4-methylpiperazine-1-carboxylate Formate (Compound No. 27)

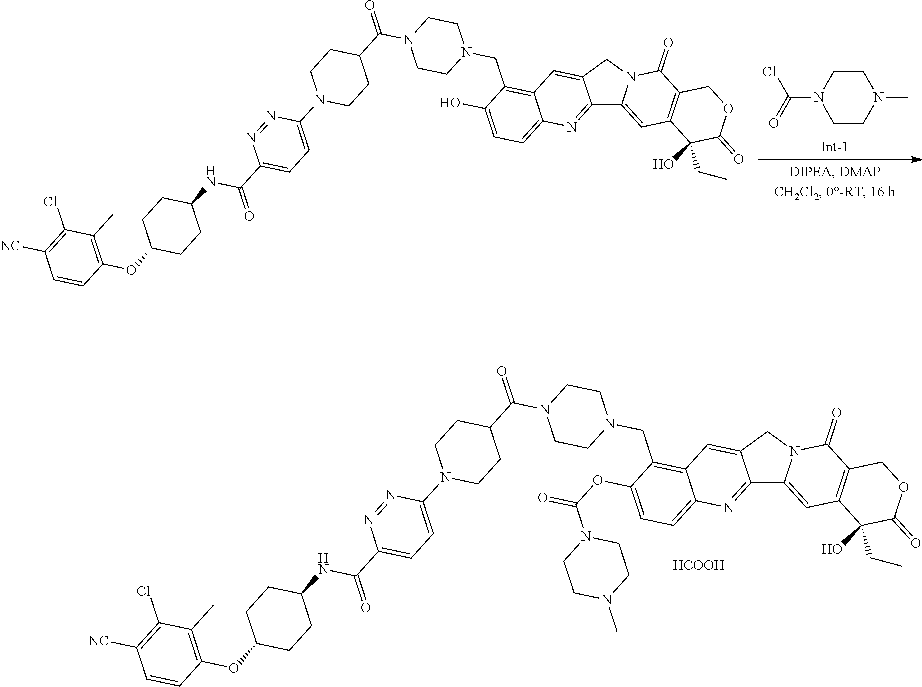

To a stirred solution of N-((1r,4r)-4-(3-chloro-4-cyano-2-methylphenoxy)cyclohexyl)-6-(4-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carbonyl)piperidin-1-yl)pyridazine-3-carboxamide (1 g, 1.06 mmol, 1.0 eq.) in DCM (18 mL) were added DIPEA (15.3 g, 3.18 mmol, 3 eq.) and DMAP (724 mg, 5.9 mmol, 0.25 eq.) followed by addition of 4-methylpiperazine-1-carbonyl chloride (Int-1, 258 mg, 15.9 mmol, 1.5 eq.) in DCM (2 mL) dropwise over a period of 10 min at 0° C. and the mixture was allowed to stir for 16 h. Progress of the reaction was monitored by TLC. After completion of the reaction, the reaction mixture was washed with water (80 mL) and extracted with DCM (3×70 mL). The combined organic extract was again washed with water (100 mL), brine (100 mL), dried over anhydrous sodium sulfate, filtered, and concentrated under vacuum to obtain the crude product. The reaction was repeated to obtain a total of 2.1 g of the crude product, which was purified by prep. HPLC method eluting with Mobile phase A: 0.1% FA in water and Mobile phase B: acetonitrile to afford 450 mg of the free base of the title compound as off white solid. The free base was converted to the formate salt using formic acid (22.7 mg, 1.1 eq.) in acetonitrile (9.2 mL) and water (9.2 mL) followed by lyophilization to provide the title compound (447 mg, 40%) as an off-white solid.

¹H NMR (400 MHz, DMSO-d₆) δ 9.05 (s, 1H), 8.57 (d, J=8.13 Hz, 1H), 8.12-8.15 (m, 2H), 7.81 (d, J=9.63 Hz, 1H), 7.77 (d, J=8.76 Hz, 1H), 7.65 (d, J=9.13 Hz, 1H), 7.33-7.37 (m, 2H), 7.26 (d, J=8.88 Hz, 1H), 6.52 (s, 1H), 5.41-5.46 (m, 2H), 5.30-5.34 (m, 2H), 4.43-4.56 (m, 3H), 3.83-3.95 (m, 3H), 3.65-3.76 (m, 2H), 3.46-3.57 (m, 4H), 3.36-3.44 (m, 2H), 3.06-3.16 (m, 2H), 2.96-3.05 (m, 2H), 2.37-2.47 (m, 6H), 2.25 (s, 3H), 2.24 (s, 3H), 2.07-2.16 (m, 2H), 1.81-1.97 (m, 5H), 1.48-1.76 (m, 9H), 0.89 (t, J=7.32 Hz, 3H). LCMS: 1068.91 [M+H]⁺. HPLC purity 95.2%.

### Example S28. Preparation of N-((1r,4r)-4-((3-Chloro-4-cyanophenyl)(methyl)amino)cyclohexyl)-6-(4-((4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)methyl)piperidin-1-yl)pyridazine-3-carboxamide (Compound No. 28)

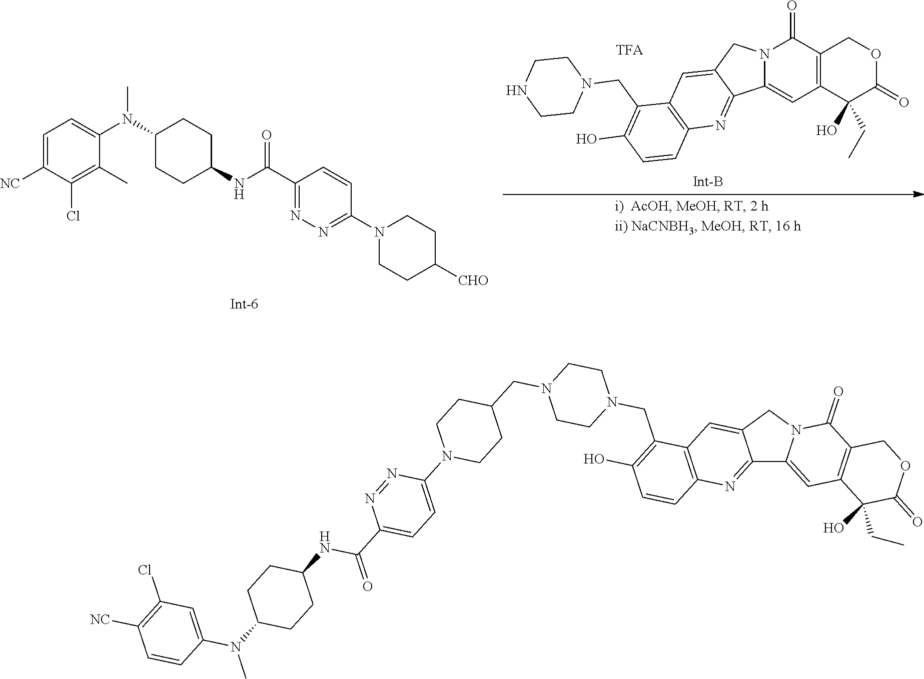 [SMILES: Cc1c(N(C)[C@H]2CC[C@H](NC(=O)c3ccc(N4CCC(C=O)CC4)nn3)CC2)ccc(C#N)c1Cl ; CC[C@@]1(O)C(=O)OCc2c1cc1n(c2=O)Cc2cc3c(CN4CCNCC4)c(O)ccc3nc2-1 ; CC[C@@]1(O)C(=O)OCc2c1cc1n(c2=O)Cc2cc3c(CN4CCN(CC5CCN(c6ccc(C(=O)N[C@H]7CC[C@H](N(C)c8ccc(C#N)c(Cl)c8)CC7)nn6)CC5)CC4)c(O)ccc3nc2-1]

To a stirred solution of N-((1r,4r)-4-((3-chloro-4-cyanophenyl)(methyl)amino)cyclohexyl)-6-(4-formylpiperidin-1-yl)pyridazine-3-carboxamide (Int-6 of Example S20, 500 mg, 1.03 mmol, 1.0 eq.) in methanol (5 mL) were added(S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione trifluoroacetate (Int-B, 622 mg, 1.35 mmol, 1.3 eq.), triethylamine (0.14 mL, 1.03 mmol, 1 eq.) and acetic acid (0.2 mL) at room temperature and allowed stirring to continue for 2 h. NaCNBH₃ (97 mg, 1.55 mmol, 1.5 eq.) was then added portion wise at 0° C. The reaction mixture was allowed to warm up to room temperature and stir for 16 h. Progress of the reaction was monitored by TLC. After completion of the reaction, cold water (50 mL) was added and extracted with 20% MeOH in DCM (2×50 mL), The combined organic extract was washed with water (60 mL), brine (50 mL) and dried over anhydrous sodium sulfate, filtered, and concentrated under vacuum. The crude obtained was purified by prep. HPLC eluting with Mobile phase A: 0.1% FA in water and Mobile phase B: acetonitrile to afford the title compound (165 mg, 17%) as a yellow solid.

¹H NMR (400 MHz, DMSO-d₆) δ 8.73 (s, 1H), 8.47 (d, J=8.31 Hz, 1H), 8.20 (s, 1H), 7.98 (d, J=9.29 Hz, 1H), 7.80 (d, J=9.29 Hz, 1H), 7.60 (d, J=8.80 Hz, 1H), 7.42 (d, J=8.80 Hz, 1H), 7.32 (d, J=9.78 Hz, 1H), 7.26 (s, 1H), 6.94 (s, 1H), 6.82 (d, J=9.29 Hz, 1H), 6.49 (s, 1H), 5.41 (s, 2H), 5.25 (s, 2H), 4.47 (d, J=12.72 Hz, 2H), 4.10 (s, 2H), 3.73-3.88 (m, 3H), 2.99 (t, J=11.98 Hz, 4H), 2.85 (s, 3H), 2.61 (s, 3H), 2.43 (s, 2H), 2.16 (d, J=6.36 Hz, 2H), 1.61-1.95 (m, 13H), 1.02-1.17 (m, 2H), 0.84-0.91 (m, 3H). LCMS: 927.4 [M+H]⁺. HPLC purity 92.7%.

### Example S29. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((4-((8S,11R,13S,14S,17S)-17-hydroxy-13-methyl-3-oxo-17-(prop-1-yn-1-yl)-2,3,6,7,8,11,12,13,14,15,16,17-dodecahydro-1H-cyclopenta[a]phenanthren-11-yl)phenyl)(methyl)amino)hexyl)piperazine-1-carboxylate (Compound No. 29)

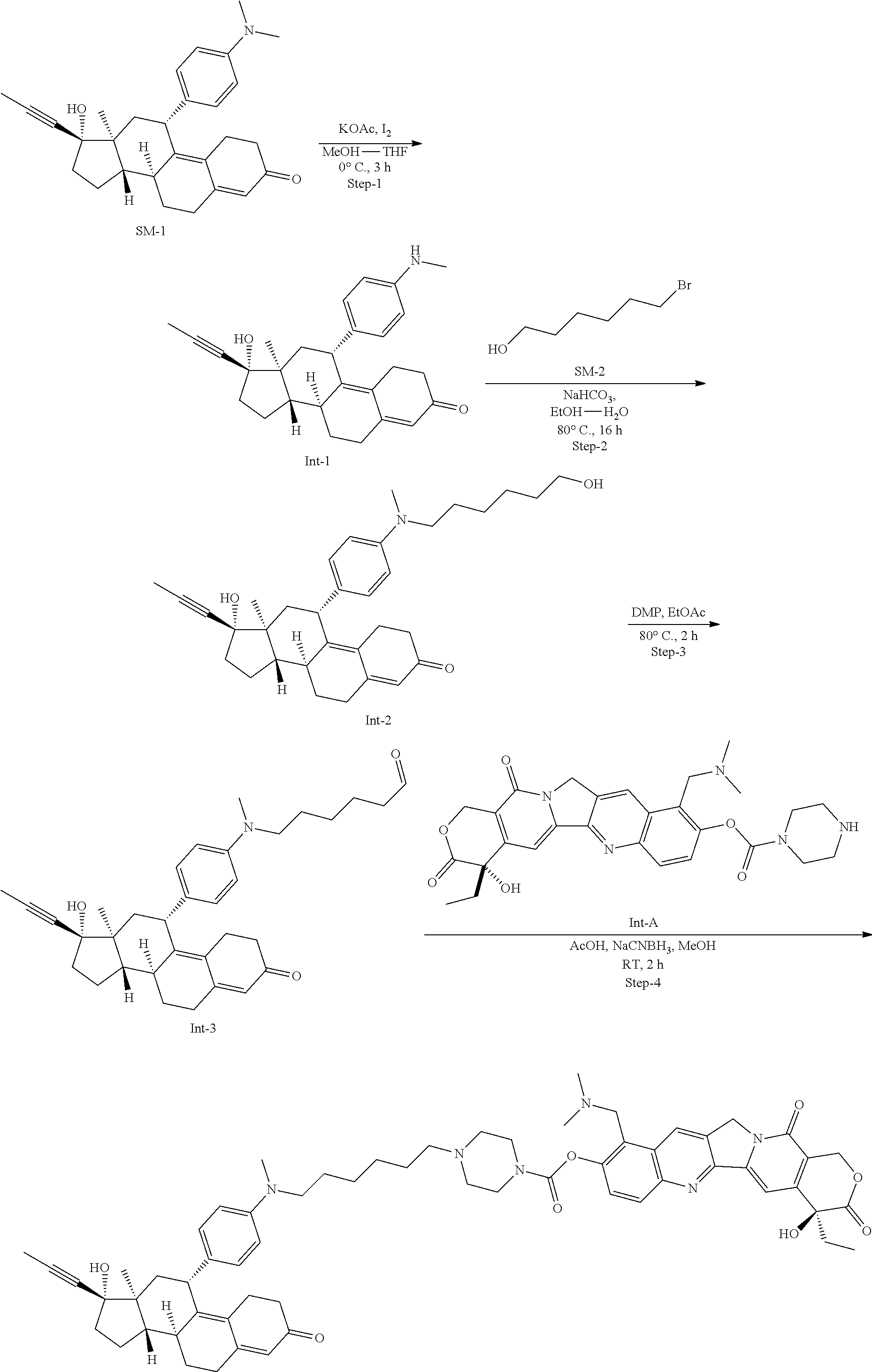

### Step-1: Preparation of (8S,11R,13S,14S,17S)-17-Hydroxy-13-methyl-11-(4-(methylamino)phenyl)-17-(prop-1-yn-1-yl)-1,2,6,7,8,11,12,13,14,15,16,17-dodecahydro-3H-cyclopenta[a]phenanthren-3-one (Int-1)

To a stirred solution of (8S,11R,13S,14S,17S)-11-(4-(dimethylamino)phenyl)-17-hydroxy-13-methyl-17-(prop-1-yn-1-yl)-1,2,6,7,8,11,12,13,14,15,16,17-dodecahydro-3H-cyclopenta[a]phenanthren-3-one (SM-1, 5 g, 11.6 mmol, 1.0 eq.) in methanol (25 mL) and THF (50 mL), KOAc (11.4 g, 116.3 mmol, 10.0 eq.) and iodine (8.8 g, 34.9 mmol, 3.0 eq.) were added at 0° C. The reaction mixture was then allowed to warm up to room temperature and stir for 3 h. Progress of the reaction was monitored by TLC. After completion of the reaction, the reaction mixture was quenched with sodium thiosulfate (Na₂S₂O₃) solution (50 g in 30 mL water) and extracted with ethyl acetate (2×200 mL). The combined organic extract was washed with brine (100 mL), dried over anhydrous sodium sulfate, filtered and concentrated under vacuum to afford (8S,11R,13S,14S,17S)-17-hydroxy-13-methyl-11-(4-(methylamino)phenyl)-17-(prop-1-yn-1-yl)-1,2,6,7,8,11,12,13,14,15,16,17-dodecahydro-3H-cyclopenta[a]phenanthren-3-one (Int-1, 5.8 g, crude) as an off-white solid which was used in next step without further purification.

¹H NMR (400 MHz, DMSO-d₆) δ 6.99 (d, J=7.82 Hz, 1H), 6.89 (d, J=7.82 Hz, 1H), 6.75 (d, J=7.82 Hz, 1H), 6.44 (d, J=8.31 Hz, 1H), 5.64 (s, 1H), 5.11 (s, 1H), 4.26-4.37 (m, 1H), 2.81-2.93 (m, 1H), 2.68-2.81 (m, 2H), 2.52-2.68 (m, 2H), 2.27-2.45 (m, 2H), 2.06-2.27 (m, 4H), 1.91-2.05 (m, 2H), 1.72-1.90 (m, 5H), 1.59 (s, 2H), 1.19-1.37 (m, 3H), 0.42 (d, J=2.93 Hz, 3H).

### Step-2: Preparation of (8S,11R,13S,14S,17S)-17-Hydroxy-11-(4-((6-hydroxyhexyl)(methyl)amino)phenyl)-13-methyl-17-(prop-1-yn-1-yl)-1,2,6,7,8,11,12,13,14,15,16,17-dodecahydro-3H-cyclopenta[a]phenanthren-3-one (Int-2)

To a solution of (8S,11R,13S,14S,17S)-17-hydroxy-13-methyl-11-(4-(methylamino)phenyl)-17-(prop-1-yn-1-yl)-1,2,6,7,8,11,12,13,14,15,16,17-dodecahydro-3H-cyclopenta[a]phenanthren-3-one (Int-1, 3 g, 7.21 mmol, 1.0 eq.) and 6-bromohexan-1-ol (SM-2, 4.7 mL, 36.09 mmol, 5.0 eq.) in ethanol (30 mL) and water (15 mL), NaHCO₃ (1.8 g, 21.6 mmol, 10.0 eq.) was added at room temperature. The reaction mixture was heated to 80° C. and stir for 16 h. Progress of the reaction was monitored by TLC. After completion of the reaction, the reaction mixture was filtered through a pad of celite bed and washed with ethyl acetate (40 mL). The filtrate was concentrated under reduced pressure, diluted with water (120 mL) and extracted with ethyl acetate (2×200 mL). The combined organic extract was washed with brine (100 mL), dried over anhydrous sodium sulfate, filtered, and concentrated under vacuum. The crude obtained was purified by Combiflash chromatography eluting with 70% ethyl acetate in heptane to afford (8S,11R,13S,14S,17S)-17-hydroxy-11-(4-((6-hydroxyhexyl)(methyl)amino)-phenyl)-13-methyl-17-(prop-1-yn-1-yl)-1,2,6,7,8,11,12,13,14,15,16,17-dodecahydro-3H-cyclopenta[a]phenanthren-3-one (Int-2, 1.4 g, 37%) as an off-white solid.

¹H NMR (400 MHz, DMSO-d₆) δ 6.97 (d, J=8.31 Hz, 2H), 6.60 (d, J=8.80 Hz, 2H), 5.65 (s, 1H), 5.07-5.10 (m, 1H), 4.26-4.35 (m, 2H), 3.34-3.41 (m, 2H), 3.15-3.28 (m, 2H), 2.71-2.85 (m, 4H), 2.53-2.66 (m, 1H), 2.29-2.44 (m, 2H), 2.10-2.28 (m, 4H), 1.92-2.05 (m, 2H), 1.74-1.87 (m, 4H), 1.56-1.66 (m, 2H), 1.36-1.54 (m, 4H), 1.29 (d, J=2.93 Hz, 7H), 0.40-0.45 (m, 3H). LCMS: 516.75.

### Step-3: Preparation of 6-((4-((8S,11R,13S,14S,17S)-17-Hydroxy-13-methyl-3-oxo-17-(prop-1-yn-1-yl)-2,3,6,7,8,11,12,13,14,15,16,17-dodecahydro-1H-cyclopenta[a]phenanthren-11-yl)phenyl)(methyl)amino)hexanal (Int-3)

To a stirred solution of (8S,11R,13S,14S,17S)-17-hydroxy-11-(4-((6-hydroxyhexyl)(methyl)amino)-phenyl)-13-methyl-17-(prop-1-yn-1-yl)-1,2,6,7,8,11,12,13,14,15,16,17-dodecahydro-3H-cyclopenta[a]phenanthren-3-one (Int-2, 1.4 g, 2.71 mmol, 1.0 eq.) in ethyl acetate (14 mL), Dess-Martin periodinane (DMP, 2.3 g, 5.43 mmol, 3.0 eq.) was added portion wise at 0° C. The reaction mixture was heated to 80° C. for 2 h. Progress of the reaction was monitored by TLC. After completion of the reaction, the reaction mixture was quenched with 50% aqueous Na₂S₂O₃ solution (10 mL), sat. NaHCO₃ solution (15 mL) and extracted with ethyl acetate (2×25 mL). The combined organic extract was washed with brine (100 mL), dried over anhydrous sodium sulfate, filtered, and concentrated under vacuum to afford 6-((4-((8S,11R,13S,14S,17S)-17-hydroxy-13-methyl-3-oxo-17-(prop-1-yn-1-yl)-2,3,6,7,8,11,12,13,14,15,16,17-dodecahydro-1H-cyclopenta[a]phenanthren-11-yl)phenyl)(methyl)amino)hexanal (Int-3, 1.6 g, crude) as a brown solid which was used in next step without further purification.

¹H NMR (400 MHz, DMSO-d₆) δ 9.65 (s, 1H), 6.96 (d, J=7.83 Hz, 2H), 6.59 (d, J=7.83 Hz, 2H), 5.64 (s, 1H), 5.09 (s, 1H), 3.99-4.07 (m, 1H), 3.20-3.25 (m, 2H), 2.70-2.78 (m, 2H), 2.53-2.61 (m, 2H), 2.37-2.44 (m, 2H), 2.27-2.35 (m, 3H), 2.12-2.25 (m, 5H), 1.94-2.01 (m, 3H), 1.77-1.85 (m, 4H), 1.57-1.66 (m, 2H), 1.52-1.56 (m, 1H), 1.44-1.51 (m, 2H), 1.22-1.35 (m, 4H), 0.41 (s, 3H). LCMS: 514.54 [M+H]⁺.

### Step-4: Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((4-((8S,11R,13S,14S,17S)-17-hydroxy-13-methyl-3-oxo-17-(prop-1-yn-1-yl)-2,3,6,7,8,11,12,13,14,15,16,17-dodecahydro-1H-cyclopenta[a]phenanthren-11-yl)phenyl)(methyl)amino)hexyl)piperazine-1-carboxylate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate (Int-A, 500 mg, 0.93 mmol, 1.0 eq.) in MeOH (5 mL, 10 vol), 6-((4-((8S,11R,13S,14S,17S)-17-hydroxy-13-methyl-3-oxo-17-(prop-1-yn-1-yl)-2,3,6,7,8,11,12,13,14,15,16,17-dodecahydro-1H-cyclopenta[a]phenanthren-11-yl)phenyl)(methyl)amino)hexanal (Int-3, 962 mg, 1.8 mmol, 2.0 eq.) and acetic acid (0.02 mL, catalytic amount) were added at 0° C. under argon atmosphere. The reaction mixture was stirred at RT for 1 h. and then NaCNBH₃ (294 mg, 4.60 mmol, 5.0 eq.) was added at 0° C. under argon atmosphere. The resulting reaction mixture was allowed to stir at room temperature until TLC indicated complete consumption of starting material. The reaction mixture was then quenched with saturated sodium bicarbonate solution (50 mL) and extracted with ethyl acetate (2×100 mL). The combined organic layer was washed with brine solution (100 mL), dried over anhydrous sodium sulfate, filtered and concentrated under reduced pressure. The crude obtain was purified by Prep. HPLC (column: spherical-C18, 40 uM, 100A; Mobile Phase A: 0.1% FA in water; Mobile Phase B: acetonitrile; flow rate: 25 mL/min, diluent: THF+DMSO). The pure fractions were combined and lyophilized under reduced pressure to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((4-((8S,11R,13S,14S,17S)-17-hydroxy-13-methyl-3-oxo-17-(prop-1-yn-1-yl)-2,3,6,7,8,11,12,13,14,15,16,17-dodecahydro-1H-cyclopenta[a]phenanthren-11-yl)phenyl)(methyl)amino)hexyl)piperazine-1-carboxylate (143 mg, 14%) as an off-white solid.

¹H NMR (400 MHz, DMSO-d₆) δ 8.93 (s, 1H), 8.07-8.13 (m, 1H), 7.61 (d, J=8.80 Hz, 1H), 7.32 (s, 1H), 6.95 (d, J=7.82 Hz, 2H), 6.59 (d, J=8.31 Hz, 2H), 6.49 (s, 1H), 5.63 (s, 1H), 5.41 (s, 2H), 5.30 (s, 2H), 5.08 (s, 1H), 4.31 (d, J=4.89 Hz, 1H), 3.74 (s, 2H), 3.68 (s, 3H), 3.45 (s, 3H), 2.82 (s, 3H), 2.74 (dd, J=9.78, 5.87 Hz, 2H), 2.41 (s, 3H), 2.32 (s, 4H), 2.12-2.21 (m, 10H), 1.93-1.99 (m, 2H), 1.84-1.88 (m, 2H), 1.75-1.82 (m, 5H), 1.57 (d, J=9.78 Hz, 2H), 1.46 (s, 5H), 1.30 (s, 6H), 0.87 (t, J=7.09 Hz, 4H), 0.41 (s, 2H). LCMS: 1031.4 [M+H]⁺.

### Example S30. Preparation of (S)-10-((4-(1-(6-(((1r,4r)-4-(3-Chloro-4-cyano-2-methylphenoxy)cyclohexyl)carbamoyl)-pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,4,5-trimethylpiperazine-1-carboxylate Formate (Compound No. 30)

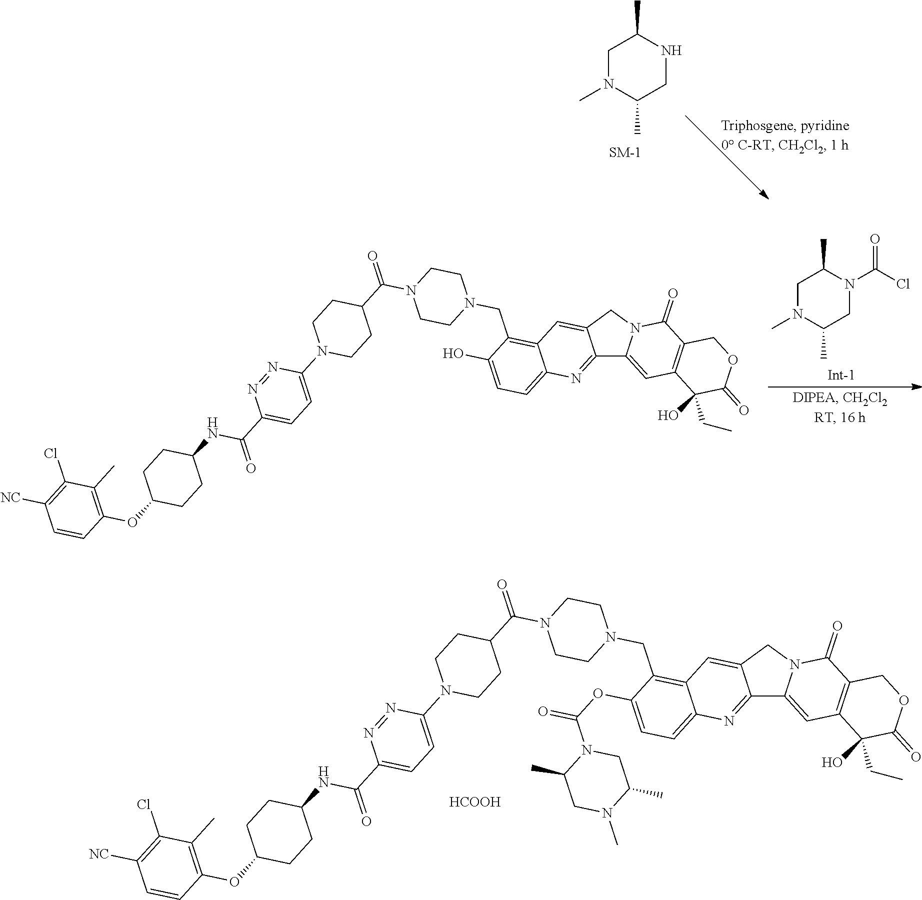

### Preparation of (2R,5S)-2,4,5-Trimethylpiperazine-1-carbonyl Chloride (Int-1)

To a stirred solution of (2S,5R)-1,2,5-trimethylpiperazine (SM-1, 500 mg, 2.29 mmol, 1.0 eq.) in DCM (7 mL) were added DIPEA (0.46 mL, 58.2 mmol, 2.5 eq.) and triphosgene (340 mg, 11.4 mmol, 0.5 eq.) solution in DCM (3 mL) dropwise over a period of 1 h min at 0° C. The reaction mixture was allowed to room temperature and stir for 16 h. Progress of the reaction was monitored by TLC (non-polar spot was observed). After completion of the reaction, the reaction mixture was used directly without any workup or purification.

### Preparation of (S)-10-((4-(1-(6-(((1r,4r)-4-(3-Chloro-4-cyano-2-methylphenoxy)cyclohexyl)carbamoyl)-pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,4,5-trimethylpiperazine-1-carboxylate Formate

To a stirred solution of N-((1r,4r)-4-(3-chloro-4-cyano-2-methylphenoxy)cyclohexyl)-6-(4-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carbonyl)piperidin-1-yl)pyridazine-3-carboxamide (500 mg, 0.53 mmol, 1.0 eq.) in DCM (6 mL) were added DIPEA (0.27 mL g, 1.59 mmol, 3 eq.) and DMAP (6.4 mg, 53 μmol, 0.1 eq.) followed by addition of (2R,5S)-2,4,5-trimethylpiperazine-1-carbonyl chloride (Int-1, 151 mg, 0.796 mmol, 1.5 eq.) in DCM (1.5 mL) dropwise over a period of 10 min at 0° C. and allowed to stir for 16 h. Progress of the reaction was monitored by TLC. The reaction mixture was then washed with water (80 mL) and extracted with DCM (3×70 mL). The combined organic extract was again washed with water (100 mL), brine (100 mL) and dried over anhydrous sodium sulfate, filtered, and concentrated under vacuum. The above reactions were repeated to get an additional 1 g of the crude product. Both crude products were combined and purified through reverse phase column to obtain 850 mg of material, which was further purified by Prep. HPLC using aqueous formic acid and acetonitrile as mobile phase to afford of (S)-10-((4-(1-(6-(((1r,4r)-4-(3-chloro-4-cyano-2-methylphenoxy)cyclohexyl)carbamoyl)-pyridazin-3-yl)piperidine-4-carbonyl)piperazin-1-yl)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,4,5-trimethylpiperazine-1-carboxylate (440 mg, 24%) as an off-white solid.

¹H NMR (400 MHz, DMSO-d₆) δ 9.05 (s, 1H), 8.56 (d, J=7.83 Hz, 1H), 8.11-8.19 (m, 1H), 7.73-7.84 (m, 2H), 7.63 (d, J=8.80 Hz, 1H), 7.35 (s, 2H), 7.26 (d, J=8.80 Hz, 1H), 6.51 (s, 1H), 5.44 (s, 2H), 5.32 (s, 2H), 4.41-4.56 (m, 3H), 4.24-4.37 (m, 1H), 3.82-3.95 (m, 3H), 3.67-3.81 (m, 1H), 3.37-3.57 (m, 5H), 3.04-3.15 (m, 3H), 2.87 (s, 2H), 2.79 (s, 1H), 2.34-2.45 (m, 3H), 2.28-2.34 (m, 1H), 2.23-2.27 (m, 7H), 2.10 (s, 2H), 1.82-1.97 (m, 4H), 1.48-1.74 (m, 8H), 1.30-1.39 (m, 3H), 0.95-1.03 (m, 2H), 0.85-0.92 (m, 3H). LCMS: 1096.81 [M+H]⁺. HPLC purity 93.4%.

### Example S31. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((1-(6-(((1r,4r)-4-(3-chloro-4-cyano-2-methylphenoxy)cyclohexyl)carbamoyl)pyridazin-3-yl)piperidin-4-yl)methyl)piperazine-1-carboxylate Formate (Compound No. 31)

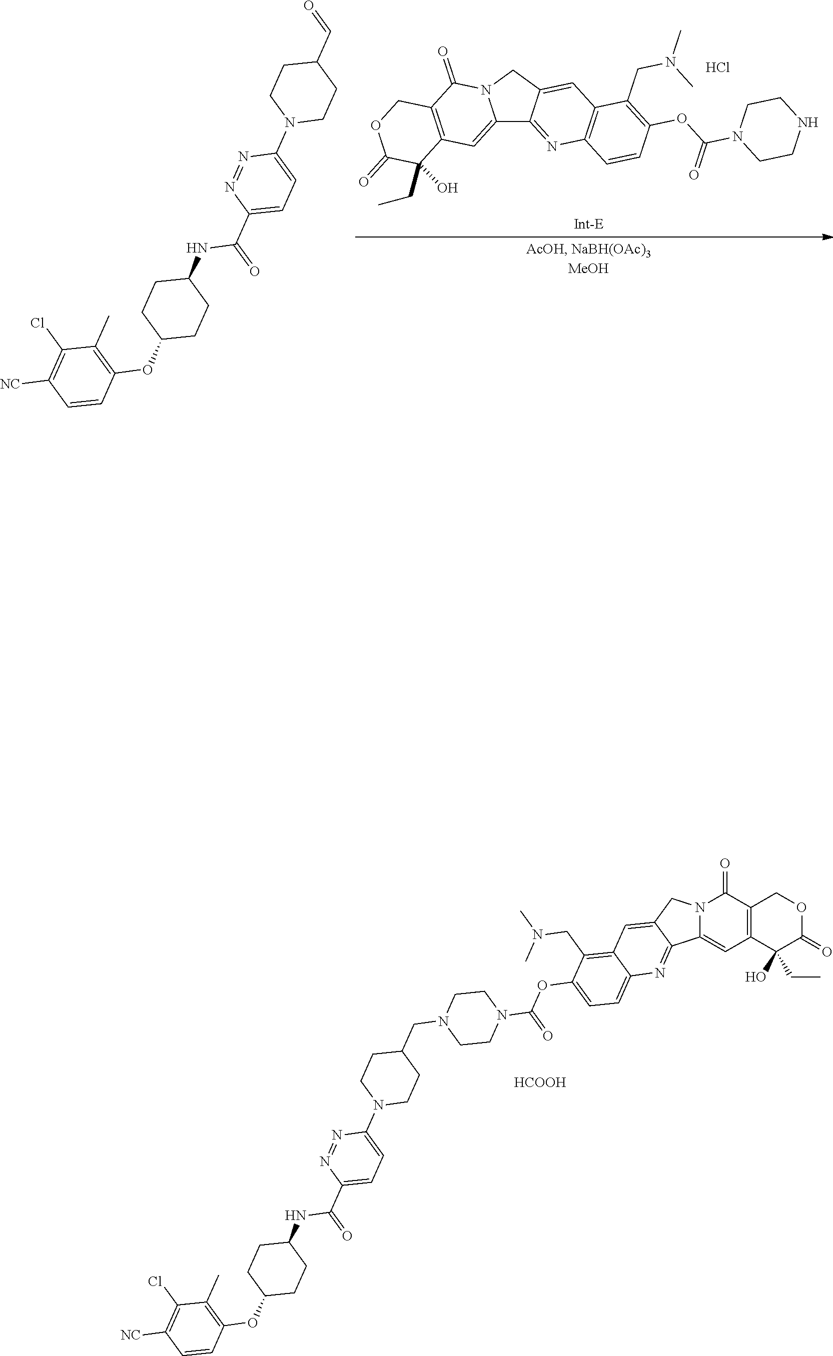

A solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate hydrochloride (Int-E, 660 mg, 12.4 mmol, 1.0 eq.) in dichloromethane (12 mL, 20 vol) and triethylamine (0.169 mL, 0.124 mmol, 1.0 eq.) was allowed to stir at RT for 15 minutes. To this mixture, N-((1r,4r)-4-(3-chloro-4-cyano-2-methylphenoxy)cyclohexyl)-6-(4-formylpiperidin-1-yl)pyridazine-3-carboxamide (Int-2, 600 mg, 4.82 mmol, 1.0 eq.) and acetic acid (148 mg, 2 eq.) were added at RT. The mixture was allowed to stir at RT for 1.5 h., after which time sodium triacetoxyborohydride (523 mg, 0.248 mmol, 2.0 eq.) was added at 0° C. under argon atmosphere, and the resulting reaction mixture was allowed to stir at RT for 16 h. Progress of the reaction was monitored by TLC. After completion of the reaction, the reaction mixture was quenched with saturated sodium bicarbonate solution (250 mL) and extracted with dichloromethane (2×80 mL), The combined organic layer was washed with brine solution (100 mL), dried over anhydrous sodium sulfate, filtered and concentrated under reduced pressure. The crude obtained was purified by Prep. HPLC (column: spherical-C18, 40 uM, 100A; Mobile Phase A: 0.1% FA in water; Mobile Phase B: acetonitrile; flow rate: 25 mL/min, diluent: THF+DMSO). The pure fractions were combined and lyophilized under reduced pressure to obtain (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((1-(6-(((1r,4r)-4-(3-chloro-4-cyano-2-methylphenoxy)cyclohexyl)carbamoyl)pyridazin-3-yl)piperidin-4-yl)methyl)piperazine-1-carboxylate formate (45 mg, 3.7%) as an off-white solid.

¹H NMR (400 MHz, DMSO-d₆) δ 8.95 (s, 1H), 8.67-8.72 (m, 1H), 8.12 (d, J=11.25 Hz, 1H), 7.75-7.84 (m, 2H), 7.61-7.67 (m, 1H), 7.32-7.40 (m, 2H), 7.17-7.22 (m, 1H), 6.48-6.53 (m, 1H), 5.41-5.45 (m, 2H), 5.32 (s, 2H), 4.81-4.85 (m, 1H), 4.44-4.54 (m, 2H), 3.87-3.99 (m, 1H), 3.83 (s, 3H), 3.45-3.54 (m, 1H), 3.17 (s, 1H), 3.03 (t, J=12.23 Hz, 3H), 2.54-2.57 (m, 1H), 2.36 (s, 5H), 2.17-2.29 (m, 7H), 1.62-2.01 (m, 13H), 1.20-1.26 (m, 3H), 1.12-1.19 (m, 2H), 0.86-0.92 (m, 3H). LCMS: 999.4 [M+H]⁺. HPLC purity 92.1%.

### Example 32. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((1-(6-(((1r,4r)-4-(3-Chloro-4-cyano-2-methylphenoxy)cyclohexyl)carbamoyl)pyridazin-3-yl)piperidin-4-yl)methyl)-2,5-dimethylpiperazine-1-carboxylate Formate (Compound No. 32)

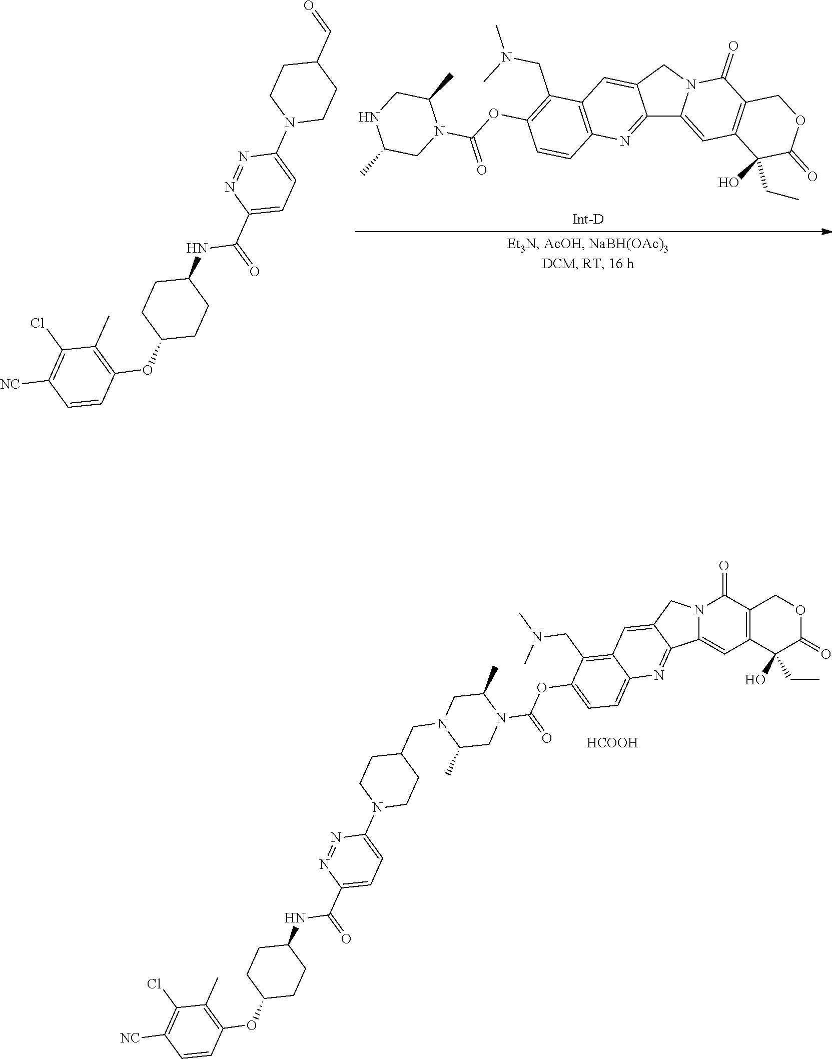

A solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-dimethylpiperazine-1-carboxylate (Int-D, 698 mg, 12.4 mmol) in dichloromethane (12 mL, 20 vol) and triethylamine (0.169 mL, 0.124 mmol) was allowed to stir at RT for 15 min. To this mixture, N-((1r,4r)-4-(3-chloro-4-cyano-2-methylphenoxy)cyclohexyl)-6-(4-formylpiperidin-1-yl)pyridazine-3-carboxamide (Int-2, 600 mg, 4.82 mmol, 1.0 eq.) and acetic acid (148 mg, 2 eq.) were added at RT and the reaction mixture was allowed to stir at RT for 2 h. To this solution, sodium triacetoxyborohydride (523 mg, 0.248 mmol, 2.0 eq.) was added at 0° C. under argon atmosphere and the resulting reaction mixture was allowed to stir at RT for 16 h until TLC indicated complete consumption of starting material. The reaction mixture was then quenched with saturated sodium bicarbonate solution (250 mL), extracted with dichloromethane (2×200 mL) and the combined organic layer washed with brine solution (200 mL), dried over anhydrous sodium sulfate, filtered and concentrated under reduced pressure. The crude obtained was purified by Prep. HPLC (column: spherical-C18, 40 uM, 100A; Mobile Phase A: 0.1% FA in water; Mobile Phase B: acetonitrile; flow rate: 25 mL/min, diluent: THF+DMSO). The pure fractions were combined and lyophilized under reduced pressure to obtain (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((1-(6-(((1r,4r)-4-(3-Chloro-4-cyano-2-methylphenoxy)cyclohexyl)carbamoyl)pyridazin-3-yl)piperidin-4-yl)methyl)-2,5-dimethylpiperazine-1-carboxylate formate (47 mg, 3.7%) as an off-white solid.

¹H NMR (400 MHz, DMSO-d₆) δ 8.95 (s, 1H), 8.72 (d, J=8.31 Hz, 1H), 8.28 (s, 1H), 8.11 (d, J=9.17 Hz, 1H), 7.79 (dd, J=12.72, 9.05 Hz, 2H), 7.62 (d, J=9.05 Hz, 1H), 7.32-7.37 (m, 2H), 7.17-7.22 (m, 1H), 6.52 (s, 1H), 5.43 (s, 2H), 5.32 (s, 2H), 4.81-4.86 (m, 1H), 4.50 (br d, J=12.72 Hz, 2H), 3.90-4.00 (m, 1H), 3.73-3.81 (m, 3H), 2.97-3.08 (m, 3H), 2.73-2.86 (m, 2H), 2.39-2.44 (m, 1H), 2.37 (s, 3H), 2.23-2.29 (m, 2H), 2.20 (s, 6H), 1.62-2.00 (m, 13H), 1.31-1.46 (m, 3H), 0.96-1.19 (m, 6H), 0.89 (t, J=7.40 Hz, 3H). LCMS: 1027.2 [M+H]⁺. HPLC purity 93.9%.

### Example S33. Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-(6-((1-(6-(((1r,4r)-4-(3-chloro-4-cyano-2-methylphenoxy)cyclohexyl)carbamoyl)pyridazin-3-yl)piperidin-4-yl)(methyl)amino)hexyl)-2,5-dimethylpiperazine-1-carboxylate Formate (Compound No. 33)

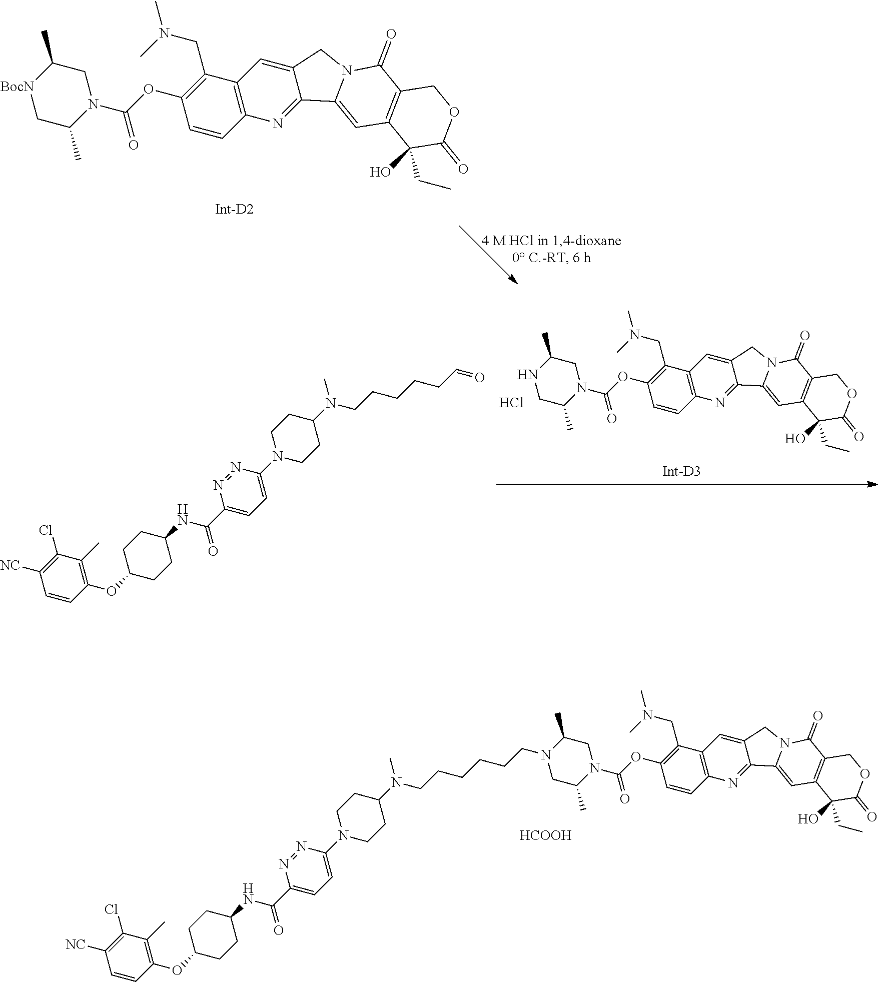 [SMILES: CC[C@@]1(O)C(=O)OCc2c1cc1n(c2=O)Cc2cc3c(CN(C)C)c(OC(=O)N4C[C@H](C)N(C(=O)OC(C)(C)C)C[C@H]4C)ccc3nc2-1 ; Cc1c(O[C@H]2CC[C@H](NC(=O)c3ccc(N4CCC(N(C)CCCCCC=O)CC4)nn3)CC2)ccc(C#N)c1Cl ; Cl ; CC[C@@]1(O)C(=O)OCc2c1cc1n(c2=O)Cc2cc3c(CN(C)C)c(OC(=O)N4C[C@H](C)NC[C@H]4C)ccc3nc2-1 ; CC[C@@]1(O)C(=O)OCc2c1cc1n(c2=O)Cc2cc3c(CN(C)C)c(OC(=O)N4C[C@H](C)N(CCCCCCN(C)C5CCN(c6ccc(C(=O)N[C@H]7CC[C@H](Oc8ccc(C#N)c(Cl)c8C)CC7)nn6)CC5)C[C@H]4C)ccc3nc2-1 ; "HCOOH"]

### Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-Dimethylpiperazine-1-carboxylate Hydrochloride (Int-D3)

To 1-(tert-butyl) 4-((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) (2S,5R)-2,5-dimethylpiperazine-1,4-dicarboxylate (Int-D2, 3.5 g, 5.29 mmol, 1.0 eq.) under nitrogen atmosphere was added 4M HCl in 1,4-dioxane (35 mL, 10 vol) at 0° C. The reaction mixture was warmed to room temperature and allowed to stir for 6h. Progress of the reaction was monitored by TLC. After completion of the reaction, volatiles were evaporated under reduced pressure and the residue was triturated with diethyl ether (3×100 mL) to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-dimethylpiperazine-1-carboxylate hydrochloride (Int-D3, 3.5 g, crude) as a yellow solid which was used directly without further purification.

LCMS: 562.53 [M+H]⁺.

### Preparation of (S)-10-((Dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-(6-((1-(6-(((1r,4r)-4-(3-chloro-4-cyano-2-methylphenoxy)cyclohexyl)carbamoyl)pyridazin-3-yl)piperidin-4-yl)(methyl)amino)hexyl)-2,5-dimethylpiperazine-1-carboxylate Formate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-dimethylpiperazine-1-carboxylate hydrochloride (Int-D3, 1.4 g, 2.34 mmol, 1 eq.) in methanol and dichloromethane (50 mL), triethylamine was added (0.35 mL, 2.51 mmol, 1.1 eq.) and the resulting reaction mixture was allowed to stir for 10 minutes. To this mixture. N-((1r,4r)-4-(3-chloro-4-cyano-2-methylphenoxy)cyclohexyl)-6-(4-(methyl(6-oxohexyl)amino)piperidin-1-yl)pyridazine-3-carboxamide (Int-4 of Example S23, 1.5 g, 2.58 mmol, 1.1 eq.) and glacial acetic acid (catalytic amount) were added at room temperature and allowed the mixture to stir for 2 h. To this reaction mixture. Na(OAc)₃BH (1.6 g, 7.55 mmol, 3.2 eq.) was added at 0° C. The reaction mixture was then allowed to warm up to room temperature and stir for 16 h. Progress of the reaction was monitored by TLC. After completion of the reaction, volatiles were evaporated under reduced pressure, quenched with ice cold water (100 mL) and extracted with 5% methanol in DCM (2×80 mL). The combined organic extract was washed with brine (100 mL), dried over anhydrous sodium sulfate, filtered, and concentrated under vacuum. The crude obtained was purified by Prep. HPLC purification method eluting with Mobile phase A: 0.1% HCOOH in water and Mobile phase B: acetonitrile to afford the title compound (400 mg, 15%) as an off-white solid.

¹H NMR (400 MHz, DMSO-d₆) δ 8.92-8.97 (m, 1H), 8.55 (d, J=7.82 Hz, 1H), 8.16 (s, 2H), 8.08-8.13 (m, 1H), 7.73-7.84 (m, 2H), 7.61 (d, J=9.78 Hz, 1H), 7.33-7.38 (m, 2H), 7.22-7.27 (m, 1H), 6.42-6.57 (m, 1H), 5.43 (s, 2H), 5.31 (s, 2H), 4.57 (s, 3H), 3.88 (s, 2H), 3.76 (s, 3H), 2.95-3.05 (m, 4H), 2.65-2.78 (m, 2H), 2.32-2.46 (m, 5H), 2.20-2.26 (m, 14H), 2.06-2.14 (m, 2H), 1.78-1.95 (m, 5H), 1.52-1.67 (m, 4H), 1.28-1.47 (m, 11H), 0.96-1.06 (m, 2H), 0.85-0.94 (m, 3H). LCMS: 1127.06 [M+H]⁺. HPLC purity 95.3%.

### Example S34. Preparation of (8S,11R,13S,14S,17R)-17-Acetyl-11-(4-(((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9, 12,15-pentaoxo-3-oxa-5,8,11,14-tetraazahexadecan-16-yl)(methyl)amino)phenyl)-13-methyl-3-oxo-2,3,6,7,8,11,12,13,14,15,16,17-dodecahydro-1H-cyclopenta[a]phenanthren-17-yl Acetate (Compound No. 34)

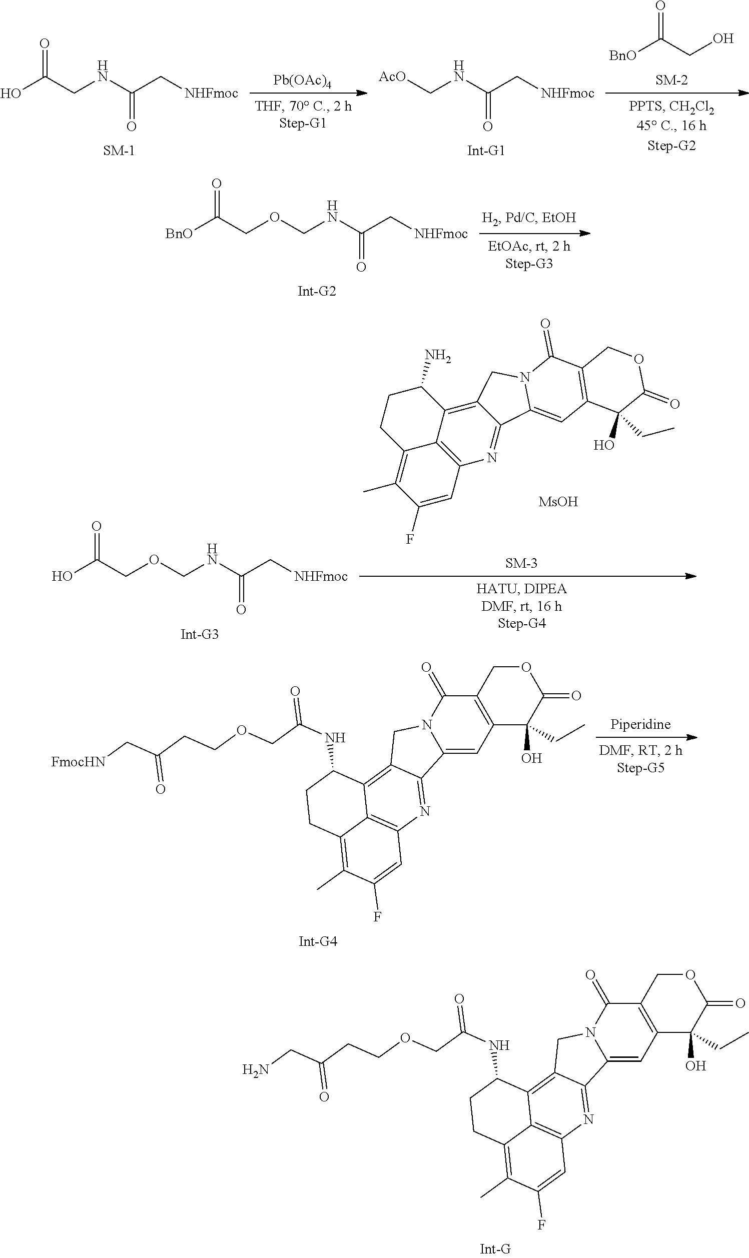

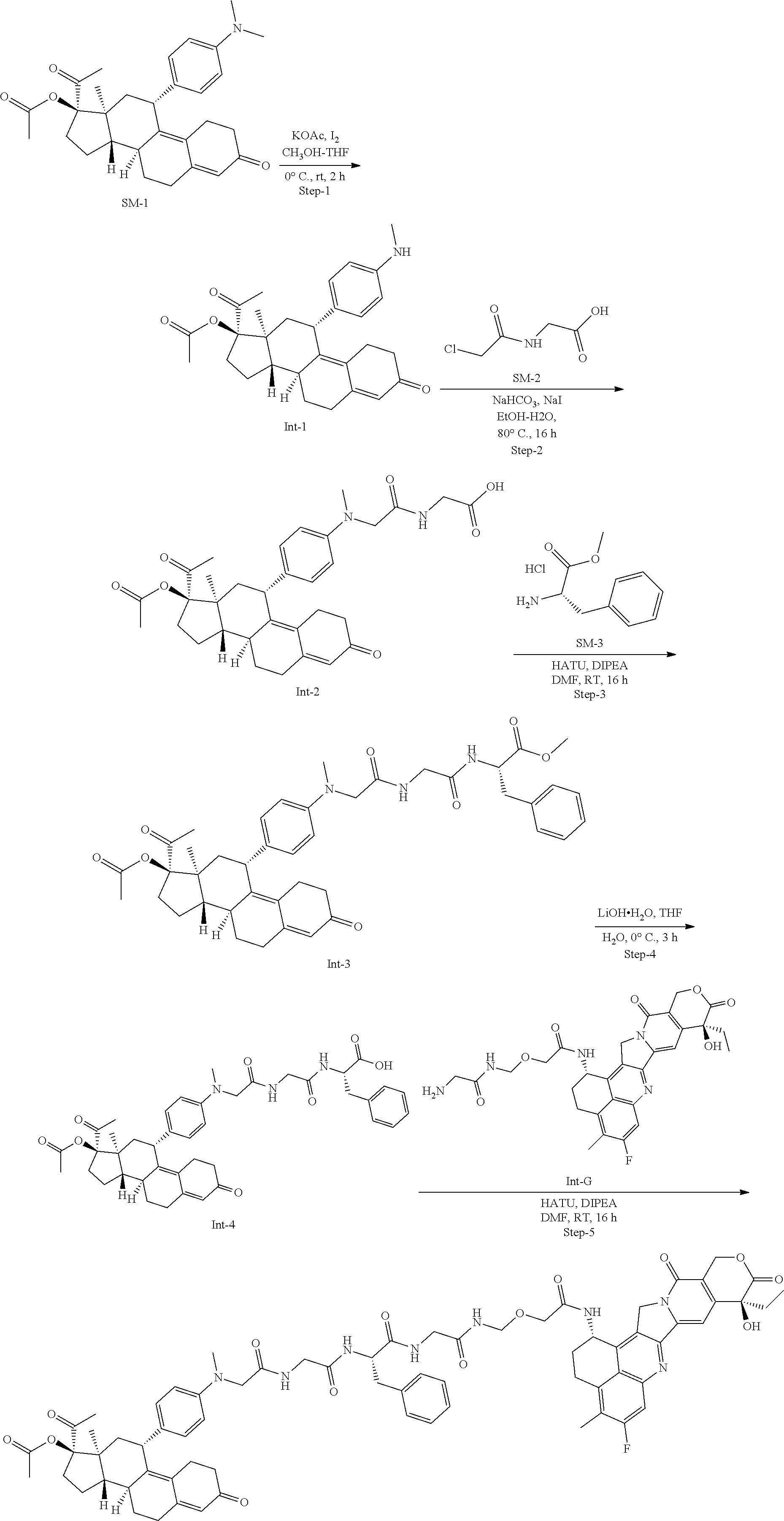

### Step-G1: Preparation of (2-((((9H-Fluoren-9-yl)methoxy)carbonyl)amino)acetamido)methyl Acetate (Int-G1)

To a solution of 2-[[2-(9H-fluoren-9-ylmethoxycarbonylamino)acetyl]amino]acetic acid (SM-1, 5 g, 14.11 mmol, 1 eq.) in tetrahydrofuran (100 mL), acetic acid (15 mL) was added at RT and the resulting mixture was made clear by warming to 40° C. Lead tetraacetate (10 g, 1.6 eq.) was added, and the resulting mixture was refluxed for about 2 h. After consumption of the starting material as indicated by TLC, reaction mixture was cooled to room temperature, filtered and washed the residue with EtOAc (200 mL). The combined filtrate was washed with 20% (w/v) aqueous trisodium citrate dihydrate solution (50 mL, 2 times). The organic layer was dried over anhydrous Na₂SO₄, filtered and concentrated under reduced pressure. The obtained crude was purified by flash column chromatography eluting with 40% EtOAc in heptane to afford (2-((((9H-fluoren-9-yl)methoxy)carbonyl)amino)acetamido)methyl acetate (Int-G1, 3.2 g, 61%) as a white solid.

¹H NMR (400 MHz, DMSO-d₆) δ 8.92 (t, J=6.60 Hz, 1H), 7.89 (d, J=7.34 Hz, 2H), 7.72 (d, J=7.34 Hz, 2H), 7.54-7.64 (m, 1H), 7.40-7.45 (m, 2H), 7.33 (t, J=7.34 Hz, 2H), 5.10 (d, J=6.85 Hz, 2H), 4.19-4.32 (m, 3H), 3.65 (d, J=6.36 Hz, 2H), 1.99 (s, 3H). LCMS: 386.2 [M+18].

### Step-G2: Preparation of Benzyl 1-(9H-Fluoren-9-yl)-3,6-dioxo-2,9-dioxa-4,7-diazaundecan-11-oate (Int-G2)

To a solution of (2-((((9H-fluoren-9-yl)methoxy)carbonyl)amino)acetamido)methyl acetate (Int-G1, 3.2 g, 8.68 mmol, 1.0 eq.) in CH₂Cl₂ (60 mL), benzyl 2-hydroxyacetate (SM-2, 8.7 g, 6.0 eq.) and PPTS (218 mg, 0.1 eq.) were added at RT, and the resulting mixture was refluxed for about 16 h. After consumption of the starting material as indicated by TLC, the solvent was evaporated under vacuum and the obtained crude was purified by flash column chromatography eluting with 20-30% EtOAc in heptane to obtain benzyl 1-(9H-fluoren-9-yl)-3,6-dioxo-2,9-dioxa-4,7-diazaundecan-11-oate (Int-G2, 2.5 g, 61%) as a white solid.

¹H NMR (400 MHz, DMSO-d₆) δ 8.66-8.78 (m, 1H), 7.89 (d, J=7.34 Hz, 2H), 7.71 (d, J=7.34 Hz, 2H), 7.57 (d, J=5.38 Hz, 1H), 7.28-7.45 (m, 9H), 5.14 (s, 2H), 4.58-4.69 (m, 2H), 4.19-4.31 (m, 3H), 4.15 (s, 2H), 3.63 (d, J=5.87 Hz, 2H). LCMS: 475.3 [M+H]⁺.

### Step-G3: Preparation of 1-(9H-Fluoren-9-yl)-3,6-dioxo-2,9-dioxa-4,7-diazaundecan-11-oic Acid (Int-G3)

To a solution of benzyl 1-(9H-fluoren-9-yl)-3,6-dioxo-2,9-dioxa-4,7-diazaundecan-11-oate (Int-G2, 1.0 g, 2.11 mmol) in EtOH (20 mL) and EtOAc (10 mL) in an autoclave, 20% (w/w) Pd/C (167 mg) was added and the reaction mixture was allowed to stir under H₂ atmosphere (50 psi) at RT for 2 h. After consumption of the starting material as indicated by TLC, the mixture was filtered through a celite plug and washed with EtOH (50 mL) and EtOAc (50 mL). The combined filtrate was concentrated under vacuum and the obtained crude was triturated with 50% solution of DCM and heptane (20 ml), and dried under vacuum to give 1-(9H-fluoren-9-yl)-3,6-dioxo-2,9-dioxa-4,7-diazaundecan-11-oic acid (Int-G3, 600 mg, 74%) as a white solid.

¹H NMR (400 MHz, DMSO-d₆) δ 8.72 (t, J=6.11 Hz, 1H), 7.89 (d, J=7.34 Hz, 2H), 7.72 (d, J=7.34 Hz, 2H), 7.58 (t, J=5.87 Hz, 1H), 7.38-7.45 (m, 2H), 7.33 (t, J=7.34 Hz, 2H), 4.60 (d, J=6.36 Hz, 2H), 4.26-4.32 (m, 2H), 4.16-4.26 (m, 2H), 3.94 (s, 2H), 3.62 (d, J=5.87 Hz, 2H). LCMS: 383.1 [M−H].

### Step-G4: Preparation of (9H-Fluoren-9-yl)methyl(2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)carbamate (Int-G4)

To a solution of 1-(9H-fluoren-9-yl)-3,6-dioxo-2,9-dioxa-4,7-diazaundecan-11-oic acid (Int-G3, 600 mg, 1.56 mmol, 1.0 eq.) and exatecan mesylate (SM-3, 830 mg, 1.56 mmol, 1.0 eq.) in DMF (18 mL), HATU (890 mg, 2.34 mmol, 1.5 eq.) and DIPEA (0.83 mL, 4.68 mmol, 3.0 eq.) were added at room temperature and the resulting mixture was stirred at same temperature for 16 h. After consumption of the starting material as indicated by TLC, the reaction mixture was poured into ice-cold water (100 mL) and extracted with EtOAc (2×100 ml). The combined organic layer was washed with ice-cold water (100 mL), dried over anhydrous Na₂SO₄, filtered, and concentrated under vacuum. The obtained crude was purified by flash column chromatography eluting with 2-3% MeOH in CH₂Cl₂ to obtain (9H-fluoren-9-yl)methyl(2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)carbamate (Int-G4, 880 mg, 70%) as an off-white solid.

¹H NMR (400 MHz, DMSO-d₆) δ 8.78 (s, 1H), 8.47 (d, J=8.77 Hz, 1H), 7.86 (d, J=7.45 Hz, 2H), 7.62-7.77 (m, 3H), 7.53 (s, 1H), 7.35-7.44 (m, 2H), 7.22-7.34 (m, 3H), 6.50 (s, 1H), 5.56 (s, 1H), 5.33-5.45 (m, 2H), 5.05-5.23 (m, 2H), 4.64 (d, J=5.70 Hz, 2H), 4.12-4.29 (m, 3H), 3.97-4.12 (m, 2H), 3.55-3.73 (m, 2H), 3.04-3.25 (m, 2H), 2.35 (s, 3H), 2.16 (s, 2H), 1.76-1.93 (m, 2H), 0.85 (t, J=6.58 Hz, 3H). LCMS: 802.5 [M+H]⁺.

### Step-G5: Preparation of 2-Amino-N-((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl) acetamide (Int-G)

To a solution of Int-G4 (150 mg, 0.19 mmol, 1.0 eq.) in DMF (3 mL), piperidine (0.19 mL, 1.9 mmol, 10.0 eq.) was added at RT and the resulting mixture was stirred for about 2 h. After consumption of the starting material as indicated by TLC, the reaction mixture was concentrated under vacuum to give 2-amino-N-((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl) acetamide (Int-G. 96 mg, crude) which was used in the next step without further purification.

LCMS: 580.4 [M+H]⁺.

### Step-1: Preparation of (8S,11R,13S,14S,17R)-17-Acetyl-13-methyl-11-(4-(methylamino)phenyl)-3-oxo-2,3,6,7,8,11,12,13,14,15,16,17-dodecahydro-1H-cyclopenta[a]phenanthren-17-yl Acetate (Int-1)

To a stirred solution of Ulipristal acetate (SM-1, 2 g, 4.20 mmol, 1.0 eq.) in methanol (15 mL) and THF (30 mL) were added KOAc (4.12 g, 42.05 mmol, 10 eq.) and iodine (5.34 g, 21.02 mmol, 5 eq.) in methanol (5 mL) and THF (10 mL) at 0° C. and the reaction mixture was allowed to warm to room temperature and stir for 2 h. Progress of the reaction was monitored by TLC. After consumption of the starting material, the reaction mixture was quenched with saturated aqueous sodium thiosulfate (Na₂S₂O₃) solution (30 mL), diluted with water (60 mL) and extracted with dichloromethane (2×100 mL). The combined organic extract was washed with brine (100 mL), dried over anhydrous sodium sulfate, filtered and concentrated under vacuum to provide Int-1 (1.1 g, 57%) as an off-white solid which was used in next step without further purification.

¹H NMR (400 MHz, DMSO-d₆) δ 6.91 (d, J=8.31 Hz, 2H), 6.44 (d, J=7.83 Hz, 2H), 5.67 (s, 1H), 4.33-4.44 (m, 1H), 3.60 (s, 1H), 2.68-2.86 (m, 2H), 2.61 (s, 4H), 2.55 (d, J=3.42 Hz, 1H), 2.29-2.41 (m, 1H), 2.05-2.24 (m, 6H), 1.80-2.05 (m, 6H), 1.60-1.80 (m, 3H), 1.22-1.45 (m, 2H), 0.24 (s, 3H).

### Step-2: Preparation of N-(4-((8S,11R,13S,14S,17R)-17-Acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycylglycine (Int-2)

To a solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 900 mg, 1.95 mmol, 1.0 eq.) and (2-chloroacetyl) glycine (SM-2, 1.48 g, 9.76 mmol, 5.0 eq.) in ethanol (10 mL) and water (10 mL), sodium iodide (1.46 g, 9.76 mmol, 5.0 eq.) and NaHCO₃ (until pH 8) were added at room temperature and the reaction mixture was heated to 80° C. 16 h. Progress of the reaction was monitored by TLC. After completion of the reaction, the reaction mixture was cooled down to room temperature and quenched with ethanolic HCl (until pH 6). The volatiles were removed under reduced pressure and the residue was diluted with water and extracted with ethyl acetate (2×70 mL). The combined organic extract was washed with brine (100 mL), dried over anhydrous sodium sulfate, filtered, and concentrated under vacuum. The crude obtained was purified by Combiflash chromatography eluting with 2-5% methanol in dichloromethane to afford Int-2 (500 mg, 44%) as a pale-yellow solid.

¹H NMR (400 MHz, DMSO-d₆) δ ppm 12.37-12.90 (m, 1H), 8.17 (br t, J=5.62 Hz, 1H), 7.00 (d, J=8.31 Hz, 2H), 6.58 (d, J=8.80 Hz, 2H), 5.67 (s, 1H), 4.36-4.43 (m, 1H), 3.84 (s, 2H), 3.71-3.81 (m, 4H), 2.92 (s, 3H), 2.52-2.79 (m, 5H), 2.28-2.41 (m, 1H), 2.12-2.23 (m, 2H), 2.10 (s. 3H), 2.00 (s, 3H), 1.60-1.78 (m, 2H), 1.16-1.44 (m, 4H), 0.23 (s, 3H). LCMS: 577.4 [M+H]⁺.

### Step-3: Preparation of Methyl N-(4-((8S,11R,13S,14S,17R)-17-Acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycylglycyl-L-phenylalaninate (Int-3)

To a solution of N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycylglycine (Int-2, 500 mg, 0.867 mmol, 1.0 eq.) and methyl L-phenylalaninate (SM-3, 224 mg, 1.04 mmol, 1.2 eq.) in DMF (10 mL), HATU (494.5 mg, 1.30 mmol, 1.5 eq.) and DIPEA (0.46 mL, 2.601 mmol, 5 eq.) were added at 0° C. and the reaction mixture was allowed to warm to room temperature and stir for 16 h. Progress of the reaction was monitored by TLC. After completion of the reaction, the reaction mixture was quenched with ice cold water (50 mL), filtered through Buchner funnel and the obtained solid compound was purified by column chromatography eluting with 1-4% methanol in dichloromethane to afford methyl N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycylglycyl-L-phenylalaninate (Int-3, 340 mg, 53%) as an off white solid.

¹H NMR (400 MHz, DMSO-d₆) δ 8.26 (d, J=7.34 Hz, 1H), 8.03 (t, J=5.62 Hz, 1H), 7.16-7.29 (m, 5H), 6.98 (d, J=8.31 Hz, 2H), 6.55 (d, J=8.31 Hz, 2H), 5.67 (s, 1H), 4.37-4.50 (m, 2H), 3.98-4.06 (m, 1H), 3.83 (s, 2H), 3.64-3.76 (m, 2H), 3.58 (s, 3H), 2.95-3.03 (m, 1H), 2.90 (s, 4H), 2.66-2.79 (m, 2H), 2.53-2.64 (m, 3H), 2.28-2.42 (m, 2H), 2.19 (d, J=4.89 Hz, 1H), 2.10 (s, 4H), 1.99 (s, 6H), 1.85-2.02 (m, 1H), 1.62-1.76 (m, 2H), 1.21 (s, 1H), 1.14-1.20 (m, 1H).

LCMS: 738.4 [M+H]⁺.

### Step-4: Preparation of N-(4-((8S,11R,13S,14S,17R)-17-Acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycylglycyl-L-phenylalanine (Int-4)

To a stirred solution of methyl N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycylglycyl-L-phenylalaninate (Int-3, 230 mg, 0.311 mmol, 1.0 eq.) in THF (20 mL) and water (3 mL) was added LiOH·H₂O (13.1 mg, 0.311 mmol, 1.0 eq.) at 0° C. and the resulting mixture was stirred at same temperature for 3 h. Progress of the reaction was monitored by TLC. After completion of the reaction, the mixture was neutralized with 1N aqueous HCl at 0° C. and extracted with ethyl acetate (2×20 mL) and 10% MeOH in DCM (2×20 mL). The combined organic layer was concentrated under reduced pressure and the obtained crude was purified by column chromatography eluting with 5-8% MeOH in DCM to afford Int-4 (180 mg, 80%) as an off white solid.

¹H NMR (400 MHz, DMSO-d₆) δ 8.09 (s, 1H), 7.68 (s, 1H), 7.08-7.21 (m, 6H), 6.98 (d, J=8.31 Hz, 2H), 6.55 (d, J=8.31 Hz, 2H), 5.63-5.76 (m, 2H), 4.38 (d, J=6.36 Hz, 1H), 4.13 (s, 1H), 3.81 (s, 1H), 3.64-3.73 (m, 2H), 3.53-3.62 (m, 2H), 3.01 (dd, J=13.45, 4.65 Hz, 2H), 2.82-2.90 (m, 3H), 2.69-2.79 (m, 2H), 2.56-2.68 (m, 2H), 2.53 (s, 1H), 2.27-2.39 (m, 1H), 2.20 (s, 1H), 2.05-2.16 (m, 4H), 1.98 (s, 3H), 1.81-1.94 (m, 2H), 1.59-1.77 (m, 2H), 1.20-1.43 (m, 3H).

LCMS: 724.5 [M+H]⁺.

### Preparation of (8S,11R,13S,14S,17R)-17-Acetyl-11-(4-(((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)(methyl)amino)phenyl)-13-methyl-3-oxo-2,3,6,7,8,11,12,13,14,15,16,17-dodecahydro-1H-cyclopenta[a]phenanthren-17-yl Acetate

To a solution of N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycylglycyl-L-phenylalanine (Int-4, 120 mg, 0.165 mmol, 1 eq.) and 2-amino-N-((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl) acetamide (Int-F, 96 mg, 0.165 mmol, 1 eq.) in DMF (4 mL), HATU (94.5 mg, 1.30 mmol, 1.5 eq.) and DIPEA (64 mg, 0.497 mmol, 3 eq.) were added at 0° C. The reaction mixture was allowed to warm up to room temperature and stir for 16 h. Progress of the reaction was monitored by TLC. After completion of the reaction, the reaction mixture was quenched with ice cold water (50 mL) and filtered through Buchner funnel. The crude obtained was purified by column chromatography using 3-7% methanol in dichloromethane to afford the title compound (120 mg, 56%) as an off white solid.

¹H NMR (400 MHz, DMSO-d₆) δ 8.62 (t, J=6.36 Hz, 1H), 8.49 (d, J=8.77 Hz, 1H), 8.33 (s, 1H), 8.09 (d, J=7.45 Hz, 1H), 8.01 (s, 1H), 7.78 (d, J=10.96 Hz, 1H), 7.31 (s, 1H), 7.11-7.25 (m, 5H), 6.97 (d, J=7.89 Hz, 2H), 6.49-6.56 (m, 3H), 5.65 (s, 1H), 5.59 (s, 1H), 5.41 (s, 2H), 5.20 (s, 2H), 4.63 (d, J=6.58 Hz, 2H), 4.43-4.51 (m, 1H), 4.38 (d, J=6.14 Hz, 1H), 4.01 (s, 2H), 3.81 (s, 2H), 3.66-3.77 (m, 3H), 3.59 (dd, J=16.66, 5.26 Hz, 1H), 3.07-3.23 (m, 3H), 2.96-3.04 (m, 1H), 2.87 (s, 3H), 2.65-2.79 (m, 3H), 2.58 (d, J=14.03 Hz, 2H), 2.35-2.41 (m, 3H), 2.29-2.34 (m, 1H), 2.17 (t, J=10.63, 5.43 Hz, 3H), 2.10 (s, 4H), 2.05-2.08 (m, 1H), 1.98 (s, 4H), 1.77-1.92 (m, 4H), 1.62-1.74 (m, 2H), 1.33-1.44 (m, 2H), 1.14-1.33 (m, 3H), 0.82-0.91 (m, 3H).

LCMS: 1286.4 [M+H]⁺. HPLC: 95.5%.

### Example S35. Preparation of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((S)-2-((S)-2-((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)amino)-3-methylbutanamido)-5-ureidopentanoyl)-2,5-dimethylpiperazine-1-carboxylate (Compound No. 35)

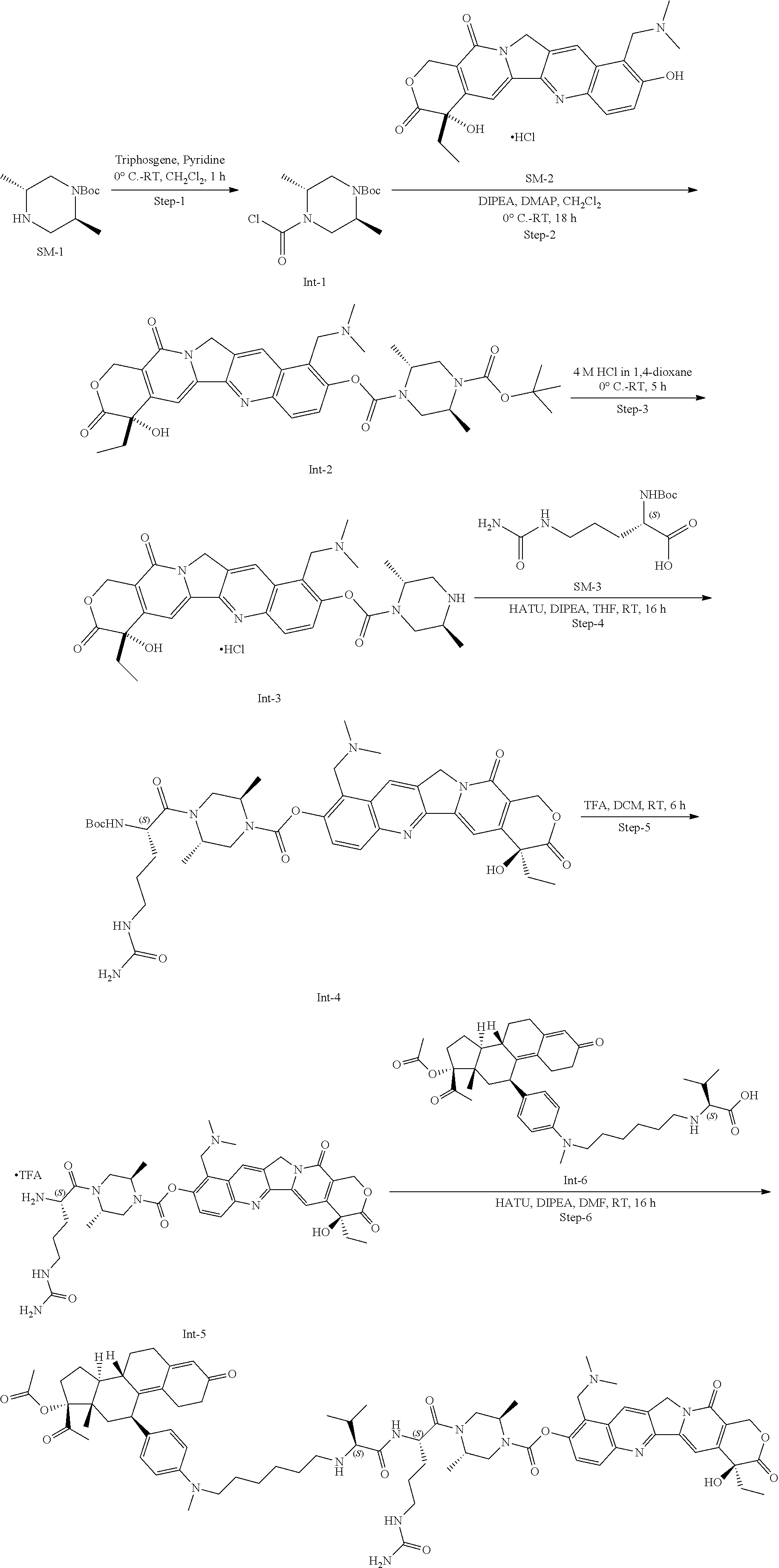

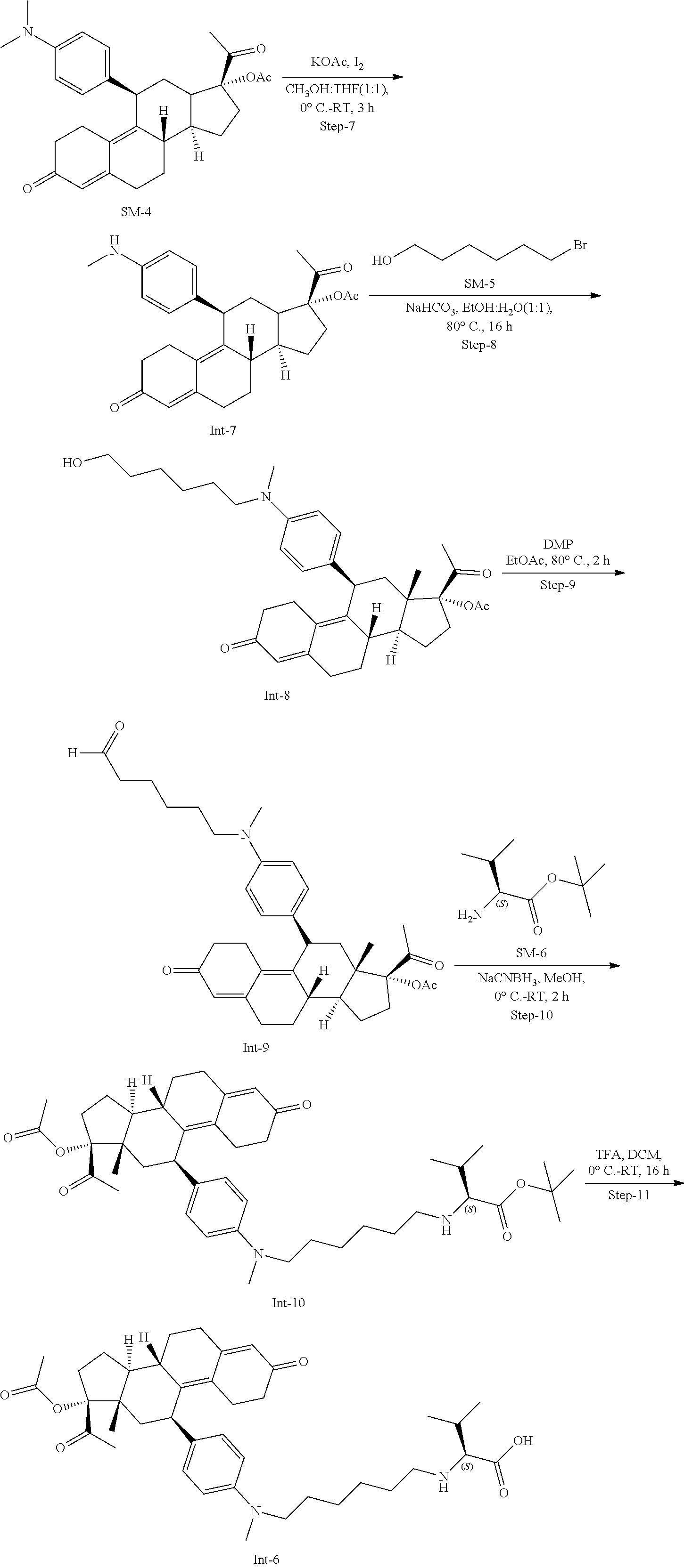

### Step-1: Synthesis of tert-butyl (2S,5R)-4-(chlorocarbonyl)-2,5-dimethylpiperazine-1-carboxylate (Int-1)

To a stirred solution of tert-butyl (2S,5R)-2,5-dimethylpiperazine-1-carboxylate (SM-1, 5 g, 23.36 mmol, 1.0 eq) in DCM (40 mL) were added pyridine (4.61 mL, 58.2 mmol, 2.5 eq) and Triphosgene (3.46 g, 11.68 mmol, 0.5 eq) solution in DCM (10 mL) drop wise over a period of 1 h at 0° C. The resultant reaction mixture was allowed to room temperature and stirred for 1 h. Progress of the reaction was monitored by TLC (non-polar spot was observed). After complete consumption of the starting material, the reaction mixture was poured into ice cold water (80 mL) and extracted with DCM (2×60 mL). The combined organic extracts were washed with brine (100 mL) and dried over anhydrous sodium sulphate, filtered, and concentrated under reduced pressure to afford tert-butyl (2S,5R)-4-(chlorocarbonyl)-2,5-dimethylpiperazine-1-carboxylate (Int-1, 6 g, crude) as a pale brown colour gum which was used in next step without any further purification.

### Step-2: Synthesis of 1-(tert-butyl) 4-((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) (2S,5R)-2,5-dimethyl piperazine-1,4-dicarboxylate (Int-2)

To a solution of (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione hydrochloride (SM-2, 6 g, 21.73 mmol, 1.5 eq) in DCM (90 mL) was added DIPEA (9.1 mL, 70.5 mmol, 5 eq) at 0° C. To this mixture was added tert-butyl (2S,5R)-4-(chlorocarbonyl)-2,5-dimethylpiperazine-1-carboxylate (Int-1, 6 g, 14.25 mmol, 1 eq) in DCM (10 mL) and DMAP (434 mg, 3.55 eq. 0.25 eq). The resultant reaction mixture was allowed to room temperature and stirred for 18 h. Progress of the reaction was monitored by TLC. After completion of starting material, the reaction mixture was quenched with water (30 mL) and extracted with ethyl acetate (2×25 mL). The combined organic extracts were washed with brine (100 mL) and dried over anhydrous sodium sulphate, filtered, and concentrated under reduced pressure to obtain crude compound, which was purified by combi flash column chromatography by eluting with EtOAc in heptane to afford 1-(tert-butyl) 4-((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) (2S,5R)-2,5-dimethyl piperazine-1,4-dicarboxylate (Int-2, 6 g, 63%) as a pale-yellow foam.

### Step-3: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-dimethylpiperazine-1-carboxylate, hydrogen chloride (Int-3)

To a stirred solution of 1-(tert-butyl) 4-((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) (2S,5R)-2,5-dimethylpiperazine-1,4-dicarboxylate (Int-2, 2 g, 3.02 mmol, 1.0 eq) in DCM (30 mL) was added 4M HCl in 1,4-Dioxane (20 mL)) at 0° C. The resultant reaction mixture was allowed to room temperature and stirred for 5 h. Progress of the reaction was monitored by TLC. After completion of starting material, the volatiles were evaporated under reduced pressure. The obtained residue was triturated with diethyl ether (2×80 mL) to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-dimethylpiperazine-1-carboxylate, hydrogen chloride (Int-3, 2.0 g. Crude) as a pale-yellow solid.

### Step-4: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((S)-2-((tert-butoxycarbonyl)amino)-5-ureidopentanoyl)-2,5-dimethylpiperazine-1-carboxylate (Int-4)

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-2,5-dimethylpiperazine-1-carboxylate·TFA salt (Int-3, 325 mg, 0.57 mmol, 1 eq) in THF (10 mL) were added DIPEA (0.3 mL, 1.73 mmol, 3 eq), HATU (326 mg, 0.86 mmol, 1.5 eq) and (S)-2-((tert-butoxycarbonyl)amino)-5-ureidopentanoic acid (SM-3, 160 mg, 0.57 mmol, 1.0 eq) at room temperature. The resultant reaction mixture was stirred at room temperature for 16 h. Progress of the reaction was monitored by TLC. After complete consumption of starting materials, the reaction mixture was poured into ice cold water (50 ml) and extracted with 10% MeOH in DCM. The combined organic layer was washed with water (50 ml), brine (50 mL) and dried over anhydrous Na₂SO₄, filtered and concentrate under reduced pressure to afford crude compound, which was purified by combi flash eluting with 10% MeOH in DCM, pure fractions were evaporated under reduced pressure to obtain (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((S)-2-((tert-butoxycarbonyl)amino)-5-ureidopentanoyl)-2,5-dimethylpiperazine-1-carboxylate (Int-4, 300 mg, 63%) as a light yellow solid.

### Step-5: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((S)-2-amino-5-ureidopentanoyl)-2,5-dimethylpiperazine-1-carboxylate. TFA (Int-5)

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((S)-2-((tert-butoxycarbonyl)amino)-5-ureidopentanoyl)-2,5-dimethylpiperazine-1-carboxylate (Int-4, 1 g, 1.22 mmol, 1.0 eq) in DCM (10 mL) was added TFA (0.9 mL, 12.22 mmol, 10.0 eq) at 0° C. under nitrogen atmosphere. The resultant reaction mixture was allowed to room temperature and stirred for 6 h. Progress of the reaction was monitored by TLC. After complete consumption of starting material, the volatiles were evaporated under reduced pressure to obtain crude compound, which was washed with diethyl ether (20 mL) and dried under vacuum to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((S)-2-amino-5-ureidopentanoyl)-2,5-dimethylpiperazine-1-carboxylate·TFA (Int-5, 1 g, Crude) as a yellow solid.

### Step-6: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((S)-2-((S)-2-((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)amino)-3-methylbutanamido)-5-ureidopentanoyl)-2,5-dimethylpiperazine-1-carboxylate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2R,5S)-4-((S)-2-amino-5-ureidopentanoyl)-2,5-dimethylpiperazine-1-carboxylate·TFA (Int-5, 500 mg, 0.69 mmol, 1 eq) in DMF (10 mL) were added DIPEA (0.6 mL, 3.48 mmol, 5 eq), HATU (397 mg, 1.04 mmol, 1.5 eq) and ((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl) (methyl)amino)hexyl)-L-valine (Int-6, 460 mg, 0.69 mmol, 1.0 eq) at room temperature. The resultant reaction mixture was stirred at room temperature for 16 h. Progress of the reaction was monitored by TLC. After complete consumption of starting materials, the reaction mixture was poured into ice cold water (50 ml). The obtained solids were filtered and washed with water, dried under vacuum. The crude solid compound was purified by Pre.HPLC, pure fractions were lyophilised to obtain the title compound (40 mg, 4%) as an off white solid.

¹H NMR (400 MHz, DMSO-d₆): δ ppm 8.95 (s, 1H), 8.24-8.30 (m, 1H), 8.18 (s, 3H), 8.12 (dd. J=9.19, 2.69 Hz, 1H), 7.60-7.69 (m, 1H), 7.34 (s, 1H), 6.97 (d, J=8.13 Hz, 2H), 6.58 (d, J=7.50 Hz, 2H), 6.48-6.55 (m, 1H), 5.87-5.95 (m, 1H), 5.66 (s, 1H), 5.43 (s, 2H), 5.37 (s, 2H), 5.32 (s, 2H), 4.81-4.90 (m, 1H), 4.55-4.78 (m, 3H), 4.35-4.41 (m, 1H), 4.27-4.35 (m, 1H), 4.15-4.25 (m, 1H), 3.95 (d, J=13.13 Hz, 1H), 3.73-3.78 (m, 2H), 3.71 (s, 1H) 3.17 (s, 1H), 2.90-3.04 (m, 5H), 2.81 (s, 4H), 2.77 (d, J=6.13 Hz, 1H), 2.73 (d, J=5.63 Hz, 1H), 2.70 (d, J=3.50 Hz, 1H), 2.60 (s, 1H), 2.33 (dt, J=3.63, 1.81 Hz, 3H), 2.17-2.22 (m, 6H), 2.12 (s, 1H), 2.09 (s, 3H), 1.98 (s, 3H), 1.85-1.91 (m, 3H), 1.60-1.81 (m, 4H), 1.34-1.48 (m, 8H), 1.17-1.33 (m, 9H), 1.03-1.12 (m, 2H), 0.82-0.92 (m, 10H), 0.22 (d, J=2.38 Hz, 2H); 98.79%; ESI MS m/z calcd. For C₇₆H₁₀₀N₁₀O₁₃ ([M+H]⁺) 1361.7; found 1361.6.

### Step-7: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-7)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-4, 10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The resultant reaction mixture was allowed to room temperature and stirred for 3 h. Progress of the reaction was monitored by TLC. After complete consumption of starting material, the reaction mixture was quenched with sodium thiosulfate (Na₂S₂O₃) solution (50 g in 30 mL water) and extracted with ethyl acetate (2×200 mL). The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-7, 8.0 g, 82%) as an off-white solid.

### Step-8: Synthesis of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthrene-17-yl acetate (Int-8)

To a solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-7, 4 g, 8.67 mmol, 1.0 eq) and 6-bromohexan-1-ol (SM-5, 7.81 g, 43.38 mmol, 5 eq) in ethanol (40 mL) and water (40 mL) was added NaHCO₃ (7.37 g, 86.76 mmol, 10 eq) at room temperature. The resultant reaction mixture was heated to 80° C. and stirred for 16 h. Progress of the reaction was monitored by TLC. After complete consumption of starting materials, the reaction mixture was filtered through a pad of celite bed and washed with ethyl acetate (40 mL). The obtained filtrate was concentrated under reduced pressure and diluted with water (120 mL) and extracted with ethyl acetate (2×200 mL). The combined organic extracts were washed with brine (100 mL) and dried over anhydrous sodium sulphate, filtered, and concentrated under reduced pressure to obtain crude compound, which was purified by Combi-flash chromatography by eluting with 70% ethyl acetate in heptane to afford (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthrene-17-yl acetate (Int-8, 2.6 g, 53%) as an off-white solid.

### Step-9: Synthesis of (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-9)

To a stirred solution of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthrene-17-yl acetate (Int-8, 500 mg, 0.891 mmol, 1 eq) in ethyl acetate (40 mL), was added Dess-Martin periodinane (DMP, 1.1 g, 2.67 mmol, 3 eq) portion wise at 0° C. The resultant reaction mixture was heated to 80° C. and stirred for 2 h. Progress of the reaction was monitored by TLC. After complete consumption of starting materials, the reaction mixture was quenched with 50% aqueous Na₂S₂O₃ solution (10 mL), sat. NaHCO₃ solution (15 mL) and extracted with ethyl acetate (2×25 mL). The combined organic extracts were washed with brine (100 mL) and dried over anhydrous sodium sulphate, filtered, and concentrated under reduced pressure to afford (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-9, 450 mg, 92%) as a brown solid.

### Step-10: Synthesis of tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl) (methyl)amino)hexyl)-L-valinate (Int-10)

To a stirred solution of tert-butyl L-valinate (SM-6, 1.23 g, 7.15 mmol) in MeOH (20 mL) were added (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-9, 2 g, 3.57 mmol) and AcOH (0.2 mL) at 0° C. The resultant reaction mixture was stirred at room temperature for 10 min. After that NaCNBH₃ (450 mg, 7.15 mmol) were added at 0° C. The resultant reaction mixture was stirred at room temperature for 2 h Progress of the reaction was monitored by TLC. After complete consumption of starting materials, the reaction mass was cooled to 0° C. and quenched with sat. NaHCO₃ solution (100 mL), extracted with 10% MeOH in DCM (2×100 mL). The combined organic layer was washed with water (50 mL) and brine (50 mL) dried over anhydrous Na₂SO₄ and concentrated under reduced pressure to obtain crude product, which was washed with diethyl ether (20 mL) and dried under vacuum to afford tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valinate (Int-10, 1.2 g, 6.4%) as a brown solid.

### Step-11: Synthesis of (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valine (Int-6)

To a stirred solution of tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valinate (Int-10, 1.2 g, 1.67 mmol, 1.0 eq) in DCM (12 mL) under nitrogen atmosphere was added TFA (12 mL, 10 vol) at 0° C., The resultant reaction mixture was warmed to room temperature and stirred for 16 h. Progress of the reaction was monitored by TLC. After complete consumption of starting materials, the volatiles were evaporated under reduced pressure to obtain crude compound, which was washed with diethyl ether (20 mL) and dried under vacuum to afford (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valine (Int-6, 900 mg, crude) as a brown solid.

### Example S36. Preparation of (8S,11R,13S,14S,17R)-17-acetyl-11-(4-((6-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)hexyl)(methyl)amino)phenyl)-13-methyl-3-oxo-(Compound No. 36)

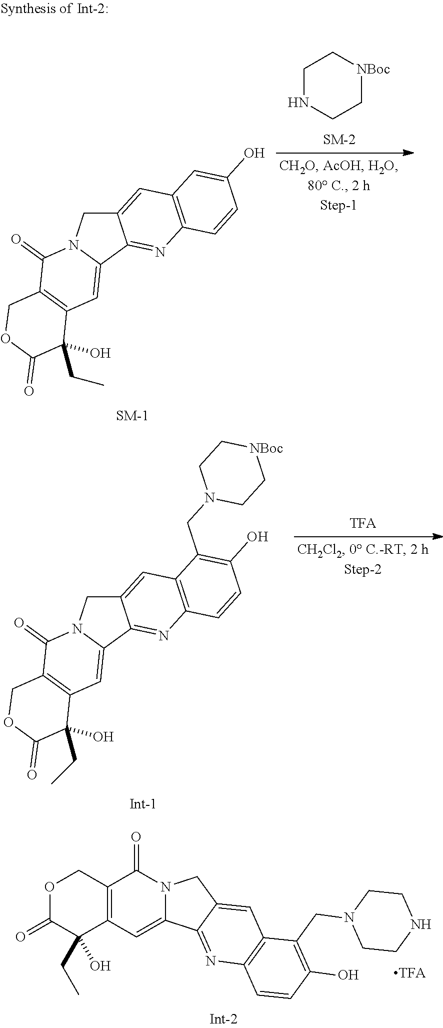 [SMILES: CC(C)(C)OC(=O)N1CCNCC1 ; CC[C@@]1(O)C(=O)OCc2c1cc1n(c2=O)Cc2cc3cc(O)ccc3nc2-1 ; CC[C@@]1(O)C(=O)OCc2c1cc1n(c2=O)Cc2cc3c(CN4CCN(C(=O)OC(C)(C)C)CC4)c(O)ccc3nc2-1 ; CC[C@@]1(O)C(=O)OCc2c1cc1n(c2=O)Cc2cc3c(CN4CCNCC4)c(O)ccc3nc2-1]

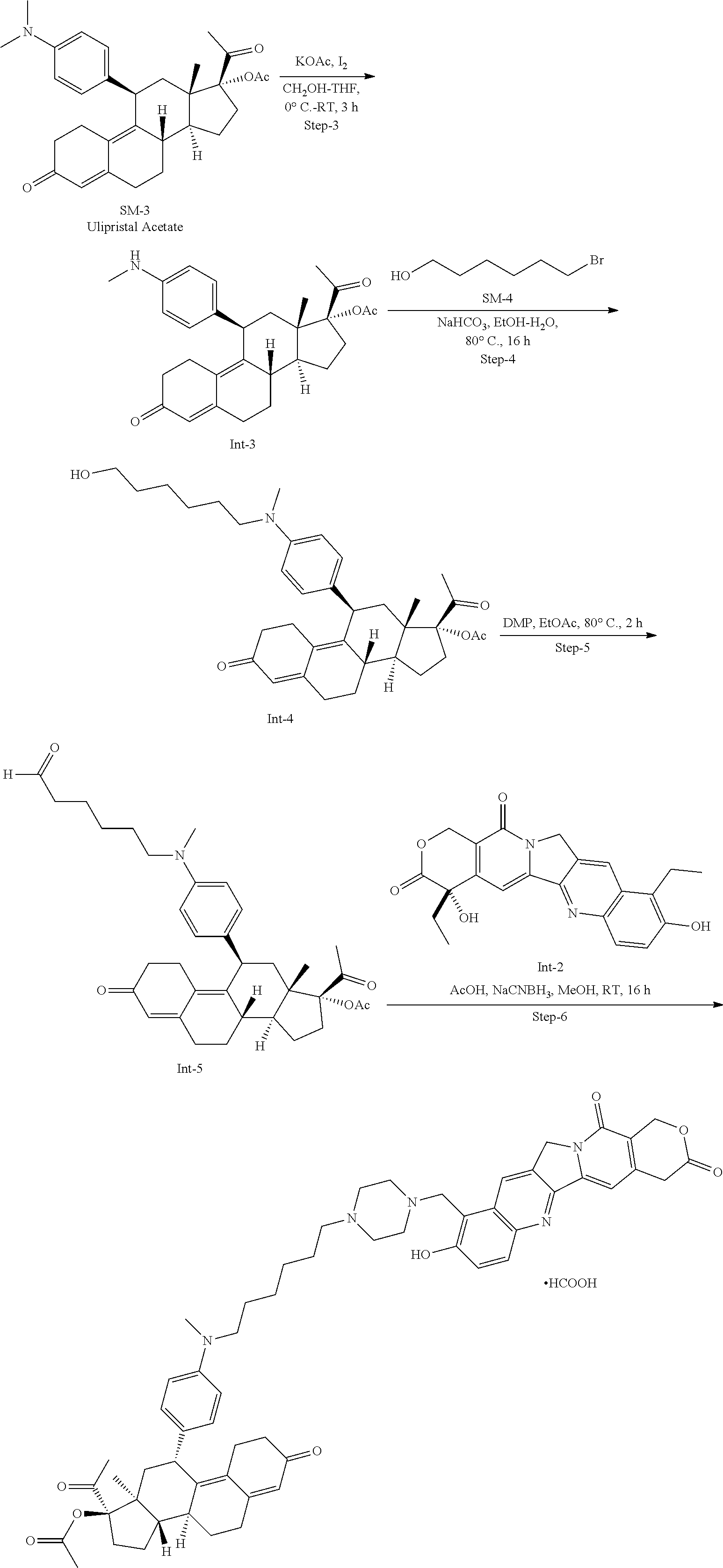

### Step-1: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-1)

To a stirred solution of (S)-4-ethyl-4,9-dihydroxy-1,12-dihydro-14H-pyrano[3′,4′:6,7]indolizino[1,2-b]quinoline-3,14(4H)-dione·HCl salt SM-1 (2.5 g, 6.86 mmol, 1.0 eq) and tert-butyl piperazine-1-carboxylate SM-2 (1.85 g, 10.3 mmol, 1.5 eq) in acetic acid (10 mL) under inert atmosphere was added 37% solution of formaldehyde (0.29 mL, 8.24 mmol, 1.2 eq) at room temperature. The reaction mixture was heated to 80° C. and stirred for 2 h in a sealed tube. Progress of the reaction was monitored by TLC. After completion of the reaction, solvents were evaporated under reduced pressure to get crude compound, which was basified with aq. ammonia until the pH reached to 9, filtered the precipitated solid compound and washed with water (10 mL) and dried under vacuum to afford Int-1 (1.9 g, 50%) as a yellow solid.

### Step-2: Synthesis of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFA salt (Int-2)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1 carboxylate Int-1 (2 g, 3.5 mmol, 1.0 eq) in DCM (20 mL) under nitrogen atmosphere was added TFA (2.7 mL, 35 mmol, 10 eq) at 0° C. Warmed to room temperature and stirred for 2 h. Progress of the reaction was monitored by TLC. After completion of the reaction, solvents were evaporated under reduced pressure, washed with diethyl ether (10 mL) and dried under vacuum to afford Int-2 (1.4 g, 83%) as a yellow solid.

### Step-3: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-3)

To a stirred solution of SM-3 (10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The reaction mixture was allowed to room temperature and stirred for 3 h. Progress of the reaction was monitored by TLC. After completion of the reaction, the reaction mixture was quenched with sodium thiosulfate (Na₂S₂O₃) solution (50 g in 30 mL water) and extracted with ethyl acetate (2×200 mL). The combined organic extracts were washed with brine (100 mL) and dried over anhydrous sodium sulphate, filtered, and concentrated under vacuum to afford Int-3 (8.0 g, 82%) as an off-white solid which was used in next step without further purification.

### Step-4: Synthesis of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-4)

To a solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate Int-3 (4 g, 8.67 mmol, 1.0 eq) and 6-bromohexan-1-ol SM-4 (7.81 g, 43.38 mmol, 5 eq) in ethanol (40 mL) and water (40 mL) was added NaHCO₃ (7.37 g, 86.76 mmol, 10 eq) at room temperature. The reaction mixture was heated to 80° C. and stirred for 16 h. Progress of the reaction was monitored by TLC. After completion of the reaction, the reaction mixture was filtered through a pad of celite bed and washed with ethyl acetate (40 mL). Filtrate was concentrated under reduced pressure, diluted with water (120 mL) and extracted with ethyl acetate (2×200 mL). The combined organic extracts were washed with brine (100 mL) and dried over anhydrous sodium sulphate, filtered, and concentrated under vacuum to obtained crude compound. The crude compound obtained was purified by combiflash chromatography by eluting with 70% ethyl acetate in heptane to afford Int-4 (2.6 g, 53%) as an off-white solid.

### Step-5: Synthesis of (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-5)

To a stirred solution of Int-4 (500 mg, 0.891 mmol, 1 eq) in ethyl acetate (40 mL), was added Dess-martin periodinane (DMP) (1.1 g, 2.67 mmol, 3 eq) portion wise at 0° C. The reaction mixture was heated to 80° C. and stirred for 2 h. Progress of the reaction was monitored by TLC. After completion of starting material, the reaction mixture was quenched with 50% aqueous Na₂S₂O₃ solution (10 mL), sat. NaHCO₃ solution (15 mL) and extracted with ethyl acetate (2×25 mL). The combined organic extracts were washed with brine (100 mL) and dried over anhydrous sodium sulphate, filtered, and concentrated under vacuum to afford Int-5 (450 mg, 92%) as a brown solid.

### Step-6: Synthesis of (8S,11R,13S,14S,17R)-17-acetyl-11-(4-((6-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)hexyl)(methyl)amino)phenyl)-13-methyl-3-oxo-2,3,6,7,8,11,12,13,14,15,16,17-dodecahydro-1H-cyclopenta[a]phenanthren-17-yl acetate

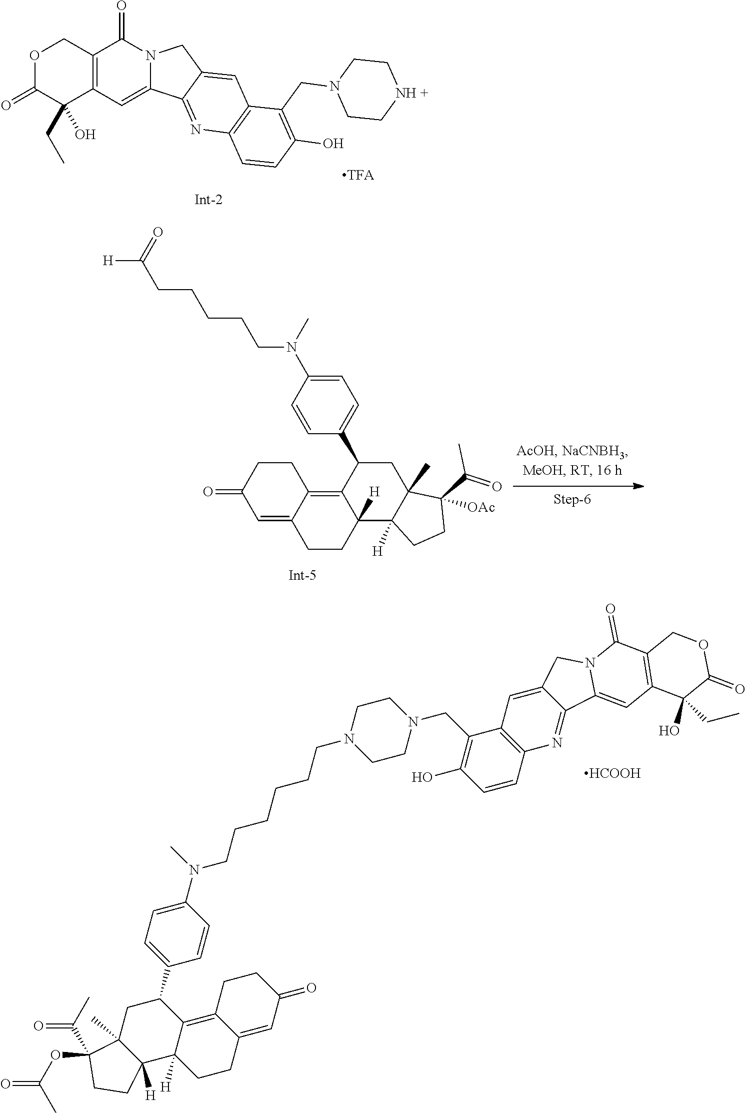

A stirred solution of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione Int-2 (500 mg, 0.868 mmol, 1 eq) and (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate Int-5 (727 mg, 1.3 mmol, 1.5 eq) in methanol (10 mL) was added triethyl amine (0.22 mL, 1.736 mmol, 2 eq) and catalytic amount of glacial acetic acid (0.1 mL, 1.736 mmol, 2 eq) at room temperature and stirred for 2 h. To this reaction mixture was added NaCNBH₃ (107 mg, 1.736 mmol, 2 eq) at 0° C. The reaction mixture was allowed to room temperature and stirred for 16 h. Progress of the reaction was monitored by TLC. After completion of the reaction, solvents were evaporated under reduced pressure, quenched with ice cold water (50 mL) and extracted with 10% methanol in DCM (2×50 mL). The combined organic extracts were washed with brine (50 mL) and dried over anhydrous sodium sulphate, filtered, and concentrated under vacuum to obtained crude compound. The crude compound obtained was purified by prep. HPLC purification method by eluting with Mobile phase A: 0.1% FA in water and Mobile phase B: Acetonitrile to afford the title compound (60 mg, 7%) as pale yellow solid.

¹H NMR (400 MHz, DMSO-d₆) δ 8.55 (s, 1H), 8.01 (s, 1H), 7.81 (d, J=8.80 Hz, 1H), 7.24 (d, J=9.29 Hz, 1H), 7.09 (s, 1H), 6.80 (d, J=8.31 Hz, 2H), 6.41 (d, J=8.31 Hz, 2H), 6.29 (br s, 1H), 5.49 (s, 1H), 5.24 (s, 2H), 5.07 (s, 2H), 4.21 (d, J=6.85 Hz, 1H), 3.93 (s, 2H), 183.05 (s, 5H), 2.52-2.71 (m, 5H), 2.13-2.25 (m, 6H), 2.02-2.11 (m, 4H), 1.97 (d, J=17.61 Hz, 3H), 1.93 (s, 3H), 1.76-1.87 (m, 5H), 1.64-1.76 (m, 4H), 1.42-1.59 (m, 3H), 1.28-1.21 (s, 5H), 1.09 (s, 5H), 0.71 (t, J=7.34 Hz, 3H), 0.06 (s, 2H); LCMS: 93.62%; ESI MS m/z calcd. For C₆₀H₇₁N₅O₉ ([M+H]⁺) 1006.53; found 1007.33; HPLC: 93.77%.

### Example S37. Preparation of (8S,11R,13S,14S,17R)-17-acetyl-11-(4-((6-(4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazin-1-yl)hexyl)(methyl)amino)phenyl)-13-methyl-3-oxo-2,3,6,7,8,11,12,13,14,15,16,17-dodecahydro-1H-cyclopenta[a]phenanthren-17-yl acetate (Compound No. 37)

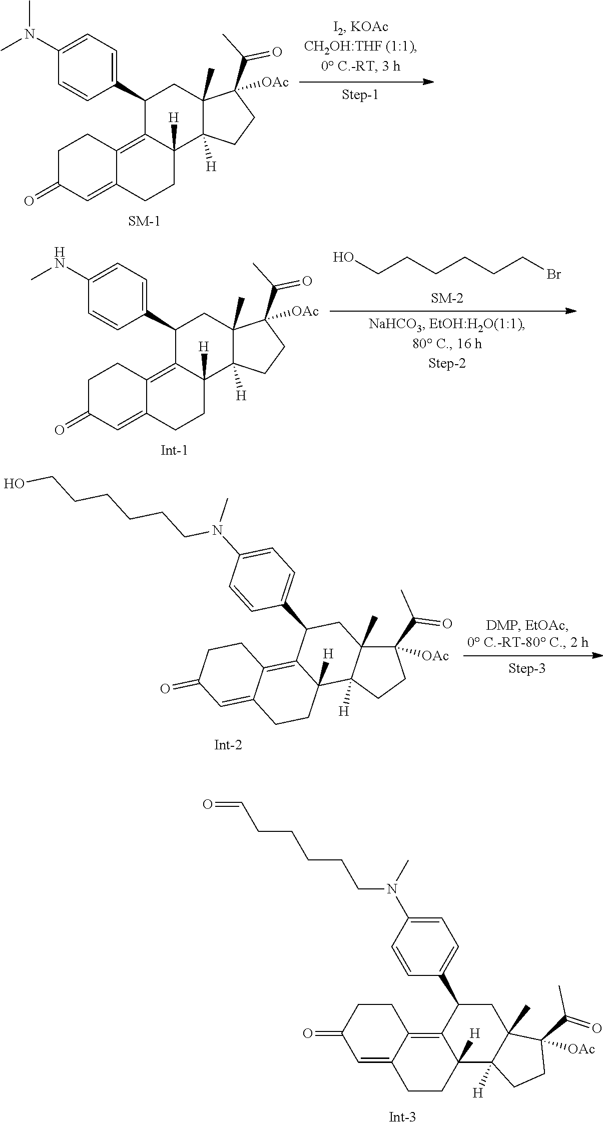

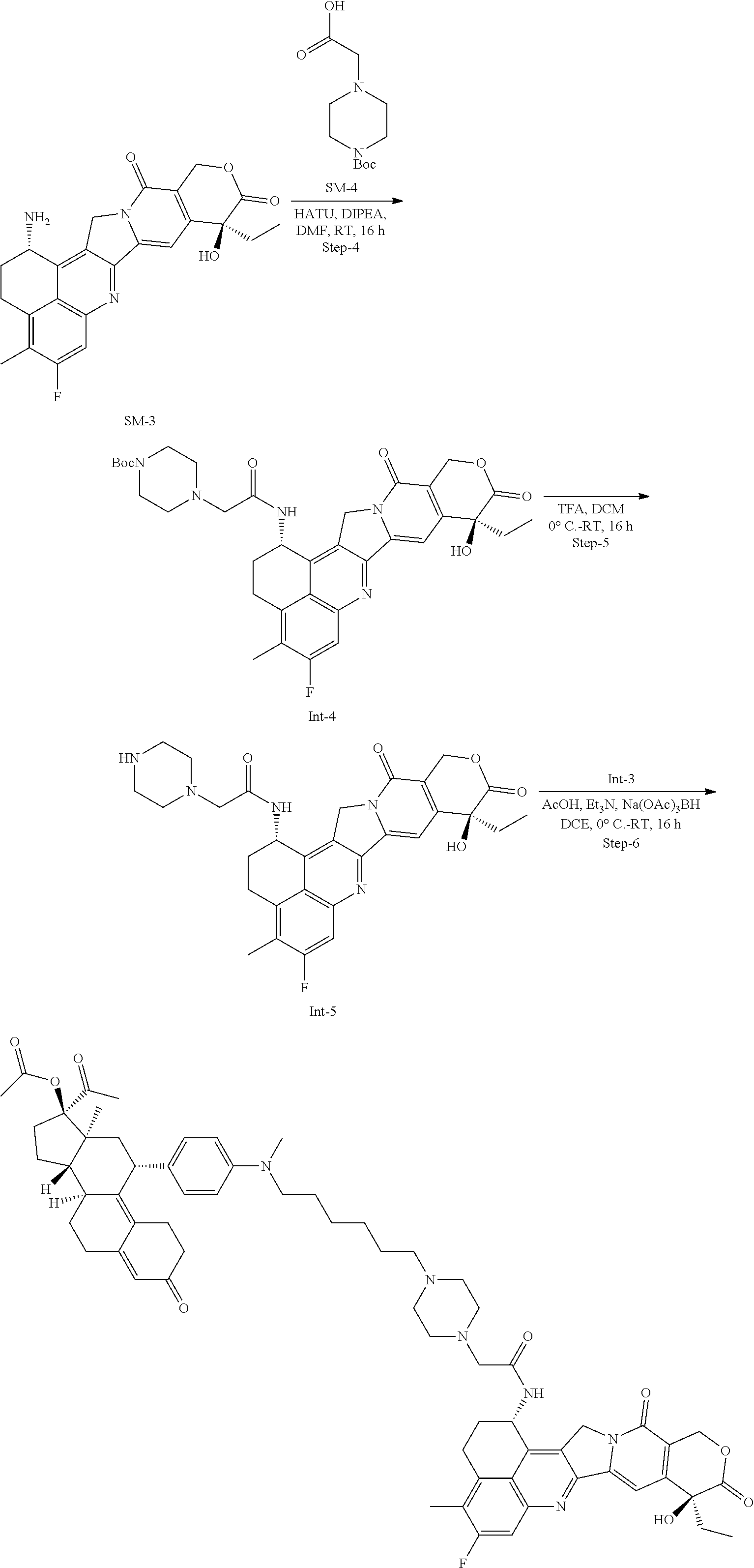 [SMILES: CC(C)(C)OC(=O)N1CCN(CC(=O)O)CC1 ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CN1CCN(C(=O)OC(C)(C)C)CC1)CC3 ; CC[C@@]1(O)C(=O)OCc2c1cc1n(c2=O)Cc2c-1nc1cc(F)c(C)c3c1c2[C@@H](NC(=O)CN1CCNCC1)CC3 ; CC[C@@]1(O)C(=O)OCc2c1cc1n(c2=O)Cc2c-1nc1cc(F)c(C)c3c1c2[C@@H](NC(=O)CN1CCN(CCCCCCN(C)c2ccc([C@H]4C[C@@]5(C)[C@@H](CC[C@]5(OC(C)=O)C(C)=O)[C@@H]5CCC6=CC(=O)CCC6=C54)cc2)CC1)CC3]

### Step-1: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-1, 10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The resultant reaction mixture was allowed to room temperature and stirred for 3 h. Progress of the reaction was monitored by TLC. After complete consumption of starting materials, the reaction mixture was quenched with sodium thiosulfate (Na₂S₂O₃) solution (50 g in 30 mL water) and extracted with ethyl acetate (2×200 mL), The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 8.0 g, 82%) as an off-white solid which was used in next step without any further purification.

### Step-2: Synthesis of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2)

To a solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 4 g, 8.67 mmol, 1.0 eq) and 6-bromohexan-1-ol (SM-2, 7.81 g, 43.38 mmol, 5 eq) in ethanol (40 mL) and water (40 mL) was added NaHCO₃ (7.37 g, 86.76 mmol, 10 eq) at room temperature. The resultant reaction mixture was heated to 80° C. and stirred for 16 h. Progress of the reaction was monitored by TLC. After complete consumption of the starting materials, the reaction mixture was filtered through a pad of celite bed and washed with ethyl acetate (40 mL). Filtrate was concentrated under reduced pressure, diluted with water (120 mL) and extracted with ethyl acetate (2×200 mL). The combined organic extracts were washed with brine (100 mL) and dried over anhydrous sodium sulphate, filtered and concentrated under reduced pressure to obtain crude compound. The crude compound was purified by combiflash chromatography by eluting with 70% ethyl acetate in heptane to afford (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 2.6 g, 53%) as an off-white solid.

### Step-3: Synthesis of (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-3)

To a stirred solution of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 500 mg, 0.891 mmol, 1 eq) in ethyl acetate (40 mL), was added dess-martin periodinane (DMP, 1.1 g, 2.67 mmol, 3 eq) portion wise at 0° C. The resultant reaction mixture was heated to 80° C. and stirred for 2 h. Progress of the reaction was monitored by TLC. After complete consumption of starting materials, the reaction mixture was quenched with 50% aqueous Na₂S₂O₃ solution (10 mL), sat. NaHCO₃ solution (15 mL) and extracted with ethyl acetate (2×25 mL). The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-3, 450 mg, 92%) as a brown solid.

### Step-4: Synthesis of tert-butyl 4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazine-1-carboxylate (Int-4)

To a stirred solution of (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (SM-3, 500 mg, 1.14 mmol, 1 eq) in DMF (10 mL) were added DIPEA (0.4 mL, 2.29 mmol, 2 eq), HATU (668 mg, 1.72 mmol, 1.5 eq) and 2-(4-tert-butoxycarbonylpiperazin-1-yl)acetic acid (SM-4, 308 mg, 1.26 mmol, 1.1 eq) at RT. The resultant reaction mixture was stirred at room temperature for 16 h. Progress of the reaction was monitored by TLC. After complete consumption of starting materials the reaction mixture was poured in ice cold water (50 mL), the obtained solids were collected via filtration and washed twice with water, dried under vacuum to afford tert-butyl 4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazine-1-carboxylate (Int-4, 525 mg, 84%) as an off white solid.

### Step-5: Synthesis of N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-(piperazin-1-yl)acetamide (Int-5)

To a stirred solution of tert-butyl 4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinoline-1-yl)amino)-2-oxoethyl)piperazine-1-carboxylate (Int-4, 525 mg, 0.79 mmol, 1 eq) in DCM (20 mL) was added TFA (0.12 mL, 2 equiv., 1.58 mmol) at 0° C. The resultant reaction mixture was stirred at room temperature for 16 h. Progress of the reaction was monitored by TLC. After complete consumption of starting materials, the volatiles were evaporated under reduced pressure to obtain N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-(piperazin-1-yl)acetamide (Int-5, 420 mg, 94%) as a green colour solid.

### Step-6: Synthesis of (8S,11R,13S,14S,17R)-17-acetyl-11-(4-((6-(4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazin-1-yl)hexyl)(methyl)amino)phenyl)-13-methyl-3-oxo-2,3,6,7,8,11,12,13,14,15,16,17-dodecahydro-1H-cyclopenta[a]phenanthren-17-yl acetate

To a stirred solution of N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-(piperazin-1-yl)acetamide (Int-5, 300 mg, 0.53 mmol, 1 eq) in DCE (10 mL) was added Et₃N (0.14 mL, 1.06 mmol, 2 eq) at 0° C. The resultant reaction mixture was stirred at room temperature for 2 h. After that (8S,11R,13S,14S,17R)-17-acetyl-13-methyl-11-(4-(methyl(6-oxohexyl)amino) phenyl)-3-oxo-2,3,6,7,8,11,12,13,14,15,16,17-dodecahydro-1H-cyclopenta[a]phenanthren-17-yl acetate (Int-3, 296 mg, 0.53 mmol) was added at 0° C. The resultant reaction mixture was stirred at room temperature for 2 h. After that sodium triacetoxy borohydride (351 mg, 1.59 mmol, 3 eq) was added at 0° C. The resultant reaction mixture was stirred at room temperature for 16 h Progress of the reaction was monitored by TLC. After complete consumption of starting materials, the reaction mixture was cooled to 0° C. and quenched with sat. NaHCO₃ solution (10 mL), extracted with 10% MeOH in DCM (2×50 mL). The combined organic layers were washed with water (20 mL), brine (20 mL) and dried over anhydrous Na₂SO₄, filtered and concentrated under reduced pressure to obtain crude product as a pale brown solid, which was purified by reverse phase purification (using 0.1% FA in water eluted in 55% Acetonitrile in 0.1% FA in water) to afford the title compound (90 mg, 15%) as a white solid.

¹H NMR (400 MHz, DMSO-d₆): δ ppm 8.41 (br d, J=8.31 Hz, 1H), 7.77 (d, J=10.76 Hz, 1H), 7.29 (s, 1H), 6.95 (br d, J=7.82 Hz, 2H), 6.56 (br d, J=8.31 Hz, 3H), 5.64 (s, 1H), 5.55 (br d, J=7.34 Hz, 1H), 5.40 (s, 2H), 5.19 (s, 2H), 4.37 (br d, J=6.36 Hz, 1H), 3.05-3.22 (m, 6H), 2.79 (s, 4H), 2.53 (br s, 5H), 2.30-2.40 (m, 7H), 2.13 (br d, J=11.74 Hz, 6H), 1.98 (s, 6H), 1.81-1.91 (m, 4H), 1.61-1.73 (m, 2H), 1.40 (br d, J=10.76 Hz, 6H), 1.24 (br s, 6H), 1.08 (t, J=7.09 Hz, 1H), 0.86 (br t, J=7.09 Hz, 4H), 0.21 (s, 3H); LCMS: 95.81%; ESI MS m/z calcd. For C₆₅H₇₇FN₆O₉ ([M+H]⁺) 1105.3; found 1106.1; HPLC: 95.26%.

### Example S38. Preparation of (8S,11R,13S,14S,17R)-17-acetyl-11-(4-((6-(4-(((S)-4-ethyl-4-hydroxy-3,14-dioxo-3,4,12,14-tetrahydro-1H-pyrano[3′,4′:6,7]indolizino[1,2-b]quinolin-10-yl)methyl)piperazin-1-yl)hexyl)(methyl)amino)phenyl)-13-methyl-3-oxo-(Compound No. 38)

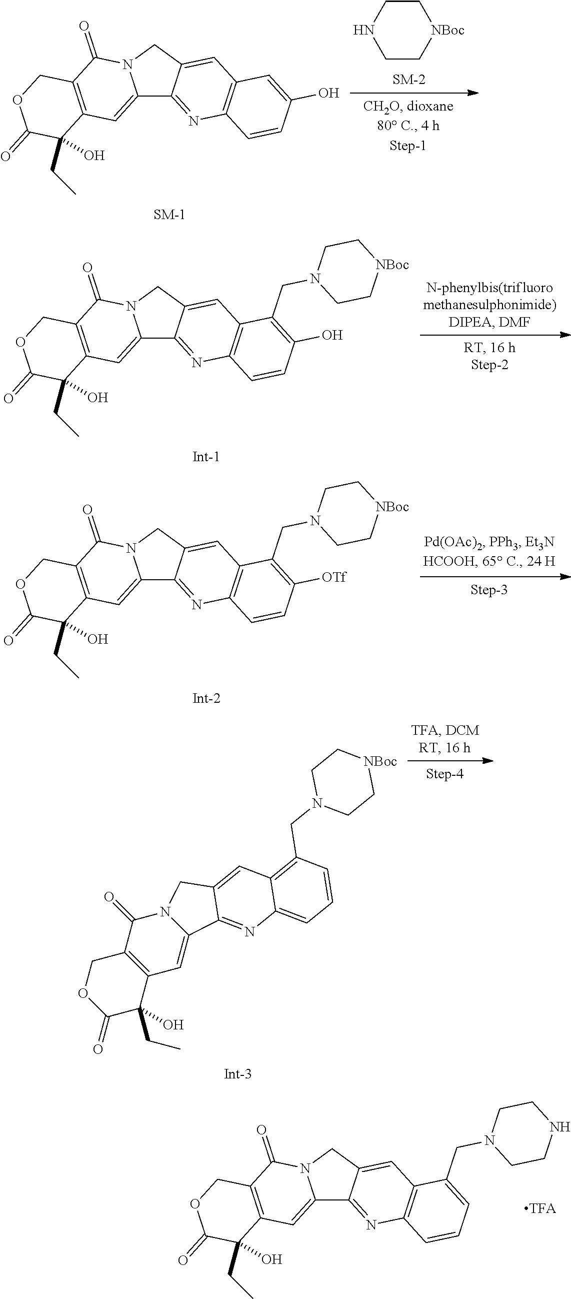 [SMILES: CC[C@@]1(O)C(=O)OCc2c1cc1n(c2=O)Cc2cc3cc(O)ccc3nc2-1 ; CC(C)(C)OC(=O)N1CCNCC1 ; CC[C@@]1(O)C(=O)OCc2c1cc1n(c2=O)Cc2cc3c(CN4CCN(C(=O)OC(C)(C)C)CC4)c(O)ccc3nc2-1 ; CC[C@@]1(O)C(=O)OCc2c1cc1n(c2=O)Cc2cc3c(CN4CCN(C(=O)OC(C)(C)C)CC4)c(OS(=O)(=O)C(F)(F)F)ccc3nc2-1 ; CC[C@@]1(O)C(=O)OCc2c1cc1n(c2=O)Cc2cc3c(CN4CCN(C(=O)OC(C)(C)C)CC4)cccc3nc2-1 ; CC[C@@]1(O)C(=O)OCc2c1cc1n(c2=O)Cc2cc3c(CN4CCNCC4)cccc3nc2-1]

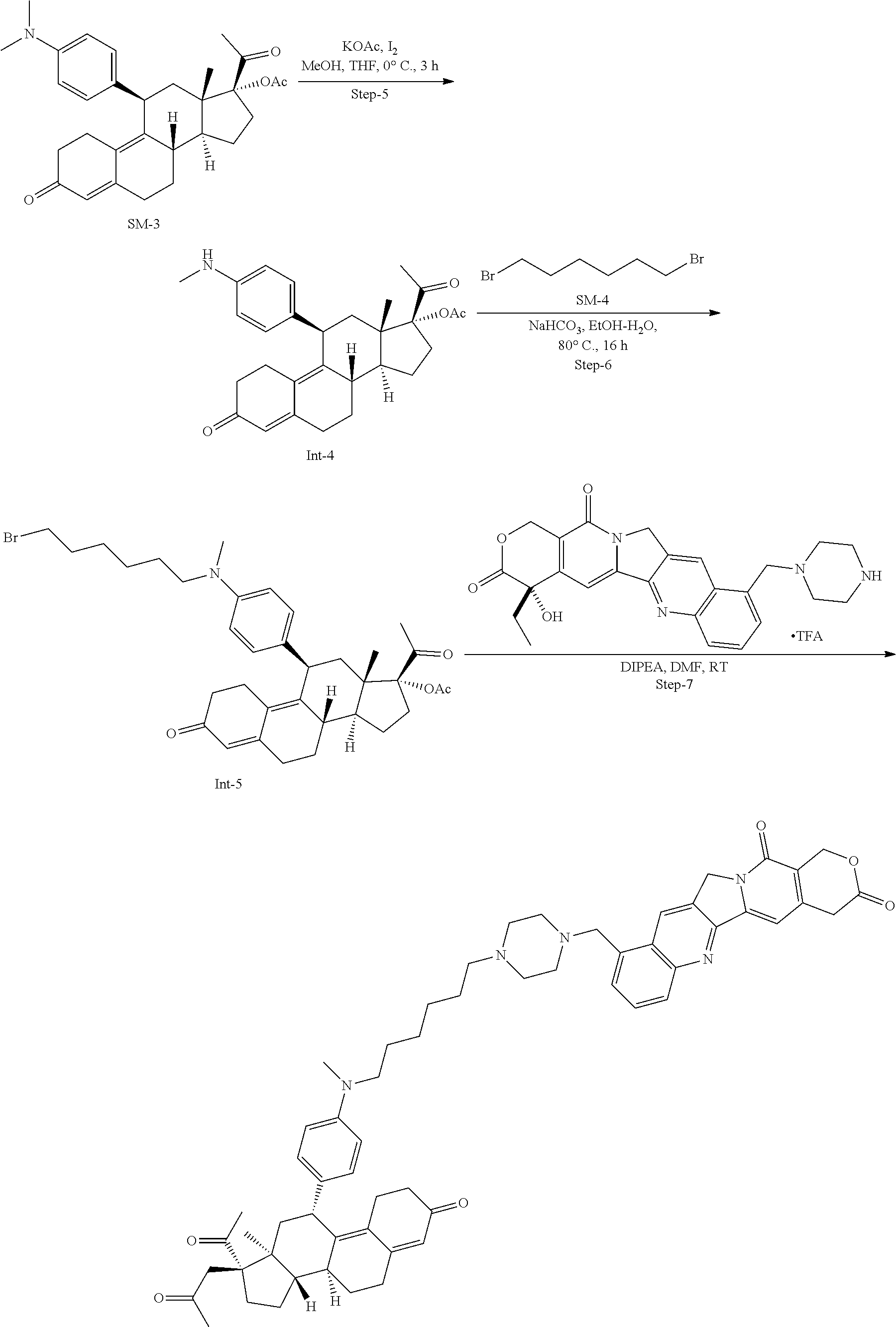

### Step-1: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-1)

A stirred solution of tert-butyl piperazine-1-carboxylate, SM-2 (3.8 g, 20.6 mmol, 1.5 eq) and aqueous formaldehyde solution (37-41%) (1.3 mL, 16.4 mmol, 1.2 eq) in 1,4-dioxane (50 mL) in a sealed tube was heated 80° C. for 1 h. Reaction mixture was cooled to room temperature, (S)-4-ethyl-4,9-dihydroxy-1,12-dihydro-14H-pyrano[3′,4′:6,7]indolizino[1,2-b]quinoline-3,14(4H)-dione, SM-1 (2.0 g, 5.49 mmol, 1 eq) was added portion wise over a period of 5 min and heating at 80° C. was continued for another 4 h. Progress of the reaction was monitored by TLC/LCMS. After completion, the reaction mixture was diluted with water (50 mL) and extracted with DCM (2×75 mL). Combined organic layers were washed with brine (50 mL), water (50 mL), dried over anhydrous Na₂SO₄ and concentrated under reduced pressure to give which was triturated with diethyl ether twice (2×50 mL) to afford Int-1 (4.8 g, 62%) as a yellow colour solid which was used for next step without further purification.

### Step-2: Synthesis of tert-butyl (S)-4-((4-ethyl-4-hydroxy-3,14-dioxo-9-(((trifluoromethyl) sulfonyl)oxy)-3,4,12,14-tetrahydro-1H-pyrano[3′,4′:6,7]indolizino[1,2-b]quinolin-10-yl)methyl)piperazine-1-carboxylate (Int-2)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate, Int-1 (2.0 g, 3.55 mmol, 1 eq), in DMF (20 mL) were added N-phenylbis(trifluoromethanesulphonimide) (1.9 g, 5.33 mmol, 1.5 eq) and DIPEA (1.85 mL, 10.67 mmol, 3 eq) dropwise over a period of 10 min. at 0° C. under nitrogen atmosphere and the reaction mixture was allowed to stir at room temperature for 16 h. Progress of the reaction was monitored by TLC/LCMS. After completion, the reaction mixture was quenched with water (50 mL) and extracted with 10% MeOH in DCM (2×75 mL). Combined organic layers were washed with brine (50 mL), water (50 mL), dried over anhydrous Na₂SO₄ and concentrated under reduced pressure to afford Int-2 (1.48 g, 61%) as an off-white solid.

### Step-3: Synthesis of tert-butyl (S)-4-((4-ethyl-4-hydroxy-3,14-dioxo-3,4,12,14-tetrahydro-1H-pyrano[3′,4′:6,7]indolizino[1,2-b]quinolin-10-yl)methyl)piperazine-1-carboxylate (Int-3)

To a stirred solution of tert-butyl (S)-4-((4-ethyl-4-hydroxy-3,14-dioxo-9-(((trifluoromethyl) sulfonyl)oxy)-3,4,12,14-tetrahydro-1H-pyrano[3′,4′:6,7]indolizino[1,2-b]quinolin-10-yl)methyl)piperazine-1-carboxylate, Int-2 (1.5 g, 2.16 mmol, 1 eq) in DMF (20 mL) were added triphenyl phosphine (56 mg, 0.21 mmol, 0.1 eq). Palladium (II) acetate (24 mg, 0.11 mmol, 0.05 eq) and triethyl amine (0.8 mL, 6.47 mmol, 3 eq) followed by dropwise addition of formic acid (0.34 mL, 8.6 mmol, 4 eq) over a period of 10 min and then the reaction mixture was heated to 65° C. and stirred for 24 h. Progress of the reaction was monitored by TLC/LCMS. After completion, the reaction mixture was quenched with water (50 mL) and extracted with 10% MeOH in DCM (2×75 mL). Combined organic layers were washed with brine (50 mL), water (50 mL), dried over anhydrous Na₂SO₄, filtered, and concentrated under reduced pressure to give crude product which was purified by combiflash column chromatography by eluting with 60% ethyl acetate in heptane to afford Int-3 (630 mg, 53%) as an off-white solid.

### Step-4: Synthesis of (S)-4-ethyl-4-hydroxy-10-(piperazin-1-ylmethyl)-1,12-dihydro-14H-pyrano[3′,4′:6,7]indolizino[1,2-b]quinoline-3,14(4H)-dione. CF₃COOH Salt

A stirred solution of tert-butyl (S)-4-((4-ethyl-4-hydroxy-3,14-dioxo-3,4,12,14-tetrahydro-1H-pyrano[3′,4′:6,7]indolizino[1,2-b]quinolin-10-yl)methyl)piperazine-1-carboxylate, Int-3 (450 mg, 0.824 mmol, 1 eq) in dichloromethane (10 mL) was cooled to 0° C. under inert atmosphere followed by addition of trifluoroacetic acid (0.64 mL, 8.24 mmol, 10 eq) and the reaction mixture was warmed to room temperature and stirred for 16 h. Progress of the reaction was monitored by TLC. After complete consumption of Int-3, solvent was evaporated to obtain crude compound which was triturated with diethyl ether (2×25 mL) to afford the TFA salt as a yellow colour solid.

### Step-5: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-4)

To a stirred solution of SM-3 (10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The reaction mixture was allowed to room temperature and stirred for 3 h. Progress of the reaction was monitored by TLC. After completion, the reaction mixture was quenched with sodium thiosulfate (Na₂S₂O₃) solution (50 g in 30 mL water) and extracted with ethyl acetate (2×200 mL). The combined organic extracts were washed with brine (100 mL) and dried over anhydrous sodium sulphate, filtered, and concentrated under vacuum to afford Int-4 (8.0 g, 82%) as an off-white solid which was used in next step without further purification.

### Step-6: Synthesis of (8S,11R,13S,14S,17R)-17-acetyl-11-(4-((6-bromohexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-5)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate. Int-4 (2.0 g, 4.34 mmol, 1.0 eq) in ethanol (20 mL) and water (5 mL) in a sealed tube was added 1,6-dibromo hexane SM-4 (3.16 g, 13.01 mmol, 3 eq) at room temperature and the reaction mixture was heated to 80° C. and stirred for 16 h. Progress of the reaction mixture was monitored by TLC/LCMS. After completion, the reaction mixture was diluted with cold water (50 mL) and extracted with ethyl acetate (2×75 mL). The combined organic extracts were washed with brine (100 mL) and dried over anhydrous sodium sulphate, filtered, and concentrated under reduced pressure to give crude product which was purified by combiflash column chromatography by eluting with 45% ethyl acetate in n-heptane to afford Int-5 (900 mg, 33%) as yellow colour liquid.

### Step-7: Synthesis of (8S,11R,13S,14S,17R)-17-acetyl-11-(4-((6-(4-(((S)-4-ethyl-4-hydroxy-3,14-dioxo-3,4,12,14-tetrahydro-1H-pyrano[3′,4′:6,7]indolizino[1,2-b]quinolin-10-yl)methyl)piperazin-1-yl)hexyl)(methyl)amino)phenyl)-13-methyl-3-oxo-2,3,6,7,8,11,12,13,14,15,16,17-dodecahydro-1H-cyclopenta[a]phenanthren-17-yl acetate

A stirred solution 8S,11R,13S,14S,17R)-17-acetyl-11-(4-((6-bromohexyl)(methyl)amino)phenyl)-13-methyl-3-oxo-2,3,6,7,8,11,12,13,14,15,16,17-dodecahydro-1H-cyclopenta[a]phenanthren-17-yl acetate, Int-5 (350 mg, 0.57 mmol, 1 eq) and (S)-4-ethyl-4-hydroxy-10-(piperazin-1-ylmethyl)-1,12-dihydro-14H-pyrano[3′,4′:6,7]indolizino[1,2-b]quinoline-3,14(4H)-dione, (345 mg, 0.63 mmol, 1.1 eq) in DMF (5 mL) was cooled to 0° C. followed by addition of DIPEA (0.3 mL, 1.72 mmol, 3 eq) dropwise over a period of 10 min and the reaction mixture was stirred at room temperature for 16 h. Progress of the reaction mixture was monitored by TLC/LCMS. After completion, the reaction mixture was quenched with cold water (50 mL) and extracted with 10% MeOH in DCM (2×75 mL), The combined organic extracts were washed with water (100 mL), brine (100 mL) and dried over anhydrous sodium sulphate, filtered, and concentrated under reduced pressure to give crude product which was purified by combiflash column chromatography by eluting with 5% MeOH in DCM to afford the title compound (85 mg, 14%) as an off-white solid.

¹H NMR (400 MHz, DMSO-d₆) δ ppm 8.99 (s, 1H), 8.08 (d, J=8.31 Hz, 1H), 7.77 (t, J=7.34 Hz, 1H), 7.61 (d, J=6.36 Hz, 1H), 7.33 (s, 1H), 6.95 (d, J=7.82 Hz, 2H), 6.45-6.63 (m, 3H), 5.64 (s, 1H), 5.42 (s, 2H), 5.28 (s, 2H), 4.36 (d, J=5.38 Hz, 1H), 3.92 (s, 2H), 3.20 (s, 2H), 2.79 (s, 3H), 2.52-2.76 (m, 4H), 2.28-2.36 (m, 3H), 2.04-2.17 (m, 8H), 1.78-2.02 (m, 12H), 1.59-1.75 (m, 3H), 1.33-1.50 (m, 6H), 1.24 (s, 6H), 0.87 (t, J=6.60 Hz, 3H), 0.21 (s, 3H); LCMS: 96.46%; ESI MS m/z calcd. For C₆₀H₇₁N₅O₈ ([M+H]⁺) 990.26; found 990.4; HPLC: 95.29%.

### Example S39. Preparation of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((S)-2-((S)-2-((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)amino)-3-methylbutanamido)-5-ureidopentanoyl)piperazine-1-carboxylate (Compound No. 39)

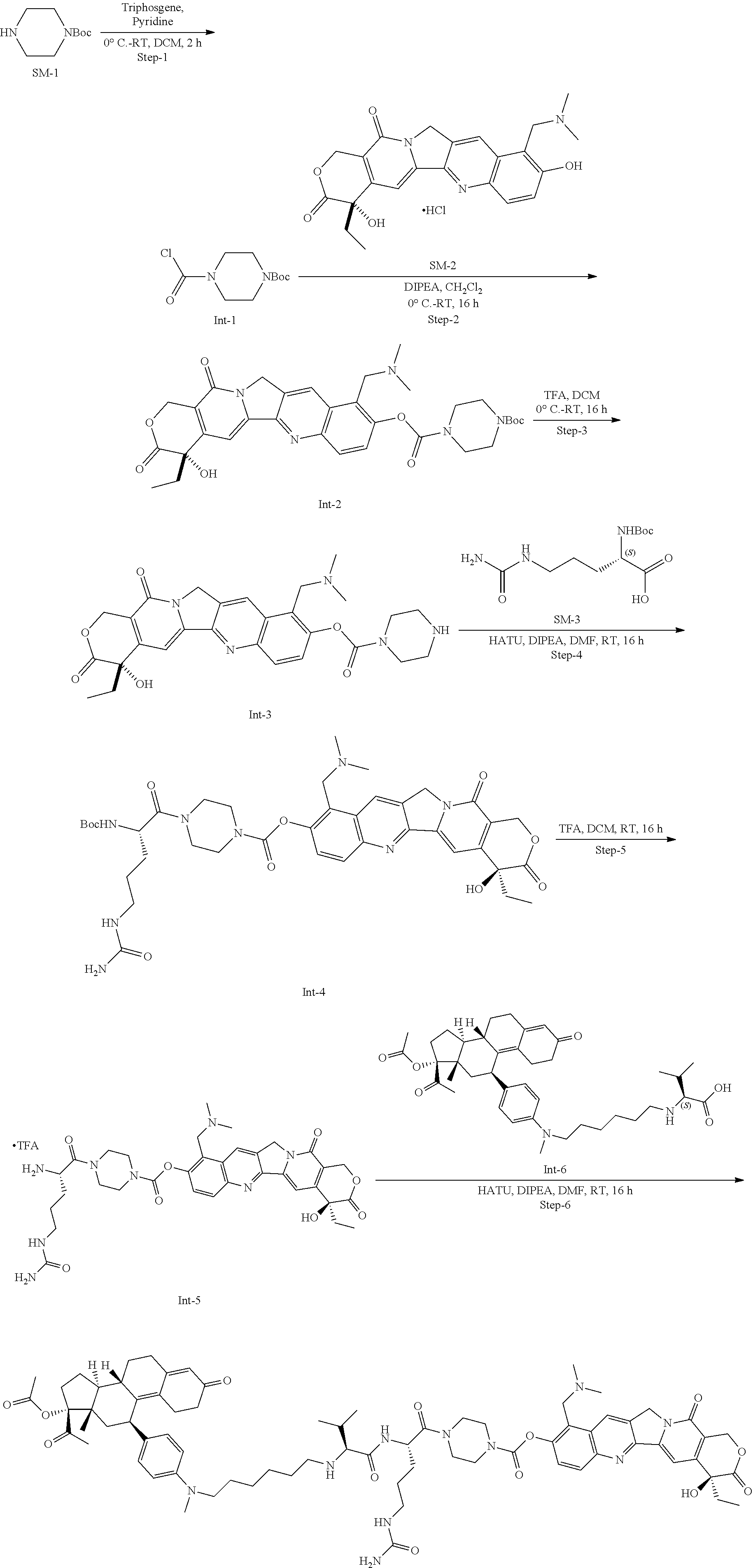

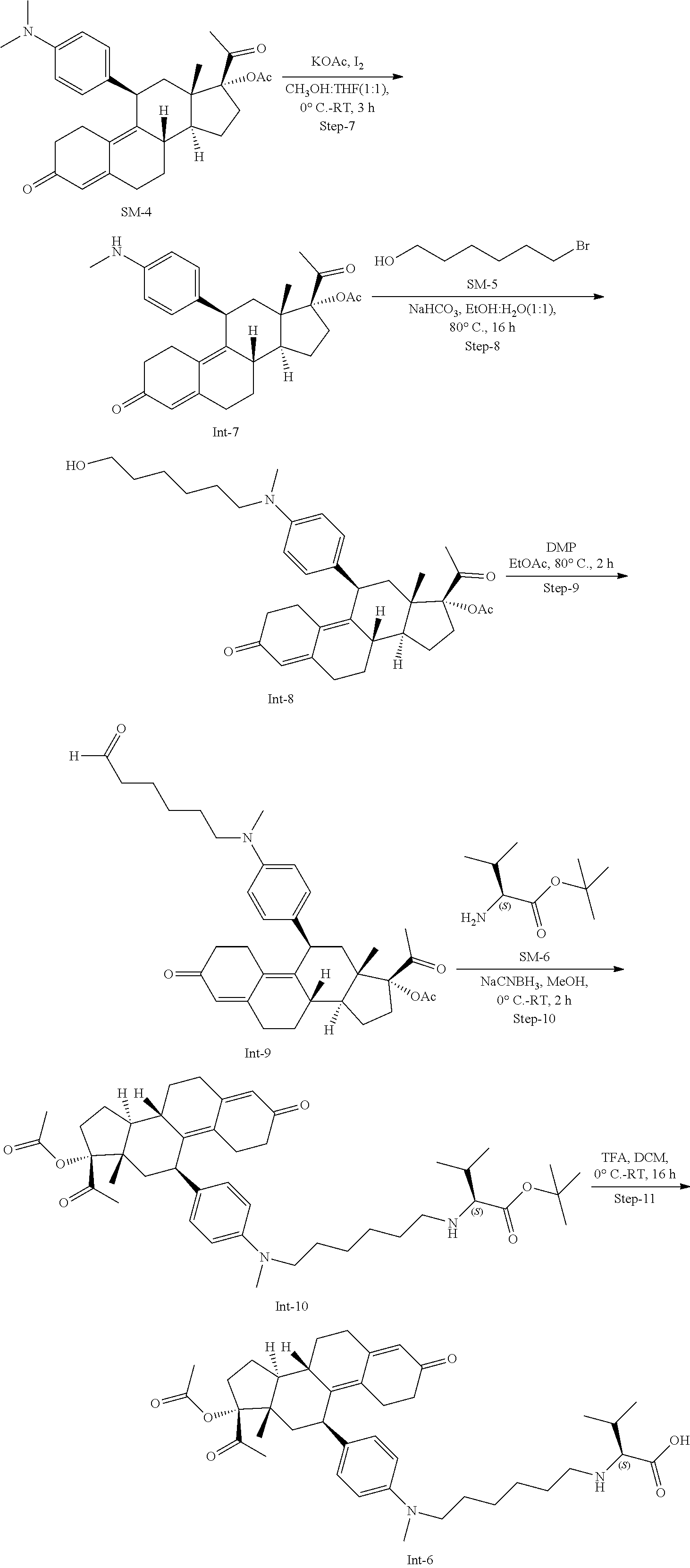

### Step-1: Synthesis of tert-butyl 4-(chlorocarbonyl)piperazine-1-carboxylate (Int-1)

To a stirred solution of tert-butyl piperazine-1-carboxylate (SM-1, 5 g, 26.8 mmol, 1.0 eq) in DCM (100 mL) were added pyridine (2.97 g, 37.6 mmol, 1.4 eq) and Triphosgene (3.19 g, 10.7 mmol, 0.4 eq) at 0° C. The resultant reaction mixture warmed to room temperature and stirred for 2 h. Progress of the reaction was monitored by TLC. After complete consumption of starting materials, the reaction mixture was diluted with water (100 mL) and extracted with DCM (2×100 mL). The combined organic extracts were washed with water (200 mL), brine (200 mL) and dried over anhydrous sodium sulphate, filtered, and concentrated under reduced pressure to afford tert-butyl 4-(chlorocarbonyl)piperazine-1-carboxylate (Int-1, 6.0 g, 90%) as a crude oil.

### Step-2: Synthesis of (S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) piperazine-1,4-dicarboxylate (Int-2)

To a stirred solution of (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione. HCl salt (SM-2, 10 g, 23.7 mmol, 1.0 eq) in DCM (250 mL) were added DIPEA (15.3 g, 118 mmol, 5 eq) and DMAP (724 mg, 5.9 mmol, 0.25 eq) followed by drop wise addition of tert-butyl 4-(chlorocarbonyl)piperazine-1-carboxylate (Int-1, 5.89 g, 23.7 mmol, 1 eq) solution in DCM over a period of 10 min at 0° C. and the resultant reaction mixture was stirred at RT for 16 h. Progress of the reaction was monitored by TLC. After complete consumption of starting materials, the reaction mixture was diluted with water (100 mL) and extracted with DCM (3×100 mL). The combined organic extracts were washed with water (200 mL), brine (200 mL) and dried over anhydrous sodium sulphate, filtered and concentrated under reduced pressure to get crude compound, which was purified by combi-flash column by using 7% methanol in DCM to afford(S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)piperazine-1,4-dicarboxylate (Int-2, 10 g, 66%) as an off white solid.

### Step-3: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate. TFA Salt (Int-3)

To a stirred solution of (S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)piperazine-1,4-dicarboxylate (Int-2, 1 g, 15 mmol, 1.0 eq) in DCM (20 mL) under nitrogen atmosphere was added TFA (3 mL) at 0° C. The resultant reaction mixture was warmed to room temperature and stirred for 16 h. Progress of the reaction was monitored by TLC. After complete consumption of starting materials, the volatiles were evaporated under reduced pressure to obtain crude product, which was diluted with sat. NaHCO₃ (50 mL) and extracted with EtOAc (2×100 mL). The combined organic layer was washed with water (100 mL), brine (100 mL)- and dried over anhydrous Na₂SO₄, filtered and evaporated under reduced pressure to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-ylpiperazine-1-carboxylate. TFA salt (Int-3, 1.02 g, crude) as an off white solid.

### Step-4: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((S)-2-((tert-butoxycarbonyl)amino)-5-ureidopentanoyl)piperazine-1-carboxylate (Int-4)

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-ylpiperazine-1-carboxylate. TFA salt (Int-3, 3 g, 5.62 mmol, 1 eq) in THF (60 mL) were added DIPEA (3.1 mL, 16.86 mmol, 3 eq), HATU (3.2 g, 8.43 mmol, 1.5 eq) and (S)-2-((tert-butoxycarbonyl)amino)-5-ureidopentanoic acid (SM-3, 3.09 g, 11.25 mmol, 2.0 eq) at room temperature. The resultant reaction mixture was stirred at room temperature for 16 h. Progress of the reaction was monitored by TLC. After complete consumption of starting materials, the reaction mixture was poured into ice cold water (50 ml) and extracted with 10% MeOH in DCM. The combined organic layer was washed with water (50 ml), brine (50 mL) and dried over anhydrous Na₂SO₄, filtered and concentrated under reduced pressure to afford crude compound, which was purified by combi-flash, eluted with 10% MeOH in DCM to obtain (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((S)-2-((tert-butoxycarbonyl)amino)-5-ureidopentanoyl)piperazine-1-carboxylate (Int-4, 3 g, 67%) as a light yellow solid.

### Step-5: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((S)-2-amino-5-ureidopentanoyl)piperazine-1-carboxylate·TFA (Int-5)

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl4-((S)-2-((tert-butoxy carbonyl)amino)-5-ureidopentanoyl)piperazine-1-carboxylate (Int-4, 3 g, 3.79 mmol, 1.0 eq) in DCM (30 mL) was added TFA (2.9 mL, 37.9 mmol, 10.0 eq) at 0° C. under nitrogen atmosphere. The resultant reaction mixture was allowed to room temperature and stirred for 6 h. Progress of the reaction was monitored by TLC. After complete consumption of starting material, the volatiles were evaporated under reduced pressure to obtain crude compound, which was washed with diethyl ether (20 mL) and dried under vacuum to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((S)-2-amino-5-ureidopentanoyl)piperazine-1-carboxylate·TFA. (Int-5, 3 g. Crude) as a yellow solid

### Step-6: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((S)-2-((S)-2-((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)amino)-3-methylbutanamido)-5-ureidopentanoyl)piperazine-1-carboxylate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((S)-2-amino-5-ureidopentanoyl)piperazine-1-carboxylate·TFA (Int-5, 1 g, 1.4 mmol, 1 eq) in DMF (10 mL) were added DIPEA (1.33 mL, 7.2 mmol, 5 eq), HATU (821 mg, 2.16 mmol, 1.5 eq) and (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valine (Int-6, 958 mg, 1.4 mmol, 1.0 eq) at room temperature. The resultant reaction mixture was stirred at room temperature for 16 h. Progress of the reaction was monitored by TLC. After complete consumption of starting materials, the reaction mixture was poured into ice cold water (50 ml). The obtained solids were filtered and washed with water, dried under vacuum. The crude solid compound was purified by Prep.HPLC, pure fractions were lyophilised to afford the title compound (67 mg, 4%) as an off white solid.

¹H NMR (400 MHz, DMSO-d₆): δ ppm 9.79-9.94 (m, 1H), 9.07-9.14 (m, 1H), 8.82-8.89 (m, 1H), 8.67-8.81 (m, 1H), 8.45-8.59 (m, 1H), 8.35 (d, J=9.17 Hz, 1H), 7.86 (d, J=9.41 Hz, 1H), 7.38 (s, 1H), 7.22 (s, 1H), 7.09 (s, 1H), 7.00 (d, J=7.95 Hz, 1H), 6.97 (s, 1H), 6.60-6.67 (m, 1H), 5.97-6.04 (m, 1H), 5.68 (s, 1H), 5.31-5.47 (m, 5H), 4.78-4.95 (m, 3H), 4.41 (d, J=6.36 Hz, 1H), 3.76 (d, J=12.23 Hz, 9H), 3.25 (t, J=6.85 Hz, 2H), 2.97-3.10 (m, 3H), 2.90 (s, 7H), 2.84 (s, 3H), 2.70-2.79 (m, 4H), 2.08-2.19 (m, 6H), 2.00 (s, 4H), 1.81-1.93 (m, 3H), 1.52-1.78 (m, 6H), 1.36-1.50 (m, 6H), 1.23-1.34 (m, 6H), 1.02 (d, J=6.85 Hz, 3H), 0.87-0.95 (m, 7H), 0.23 (s, 3H); 95.17%; ESI MS m/z calcd. For C₇₄H₉₆N₁₀O₁₃ ([M+H]⁺) 1333.7; found 1333.6; HPLC: 95.08%.

### Step-7: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-7)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-4, 10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The resultant reaction mixture was allowed to room temperature and stirred for 3 h. Progress of the reaction was monitored by TLC. After complete consumption of starting materials, the reaction mixture was quenched with sodium thiosulfate (Na₂S₂O₃) solution (50 g in 30 mL water) and extracted with ethyl acetate (2×200 mL), The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-7, 8.0 g, 82%) as an off-white solid.

### Step-8: Synthesis of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthrene-17-yl acetate (Int-8)

To a solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-7, 4 g, 8.67 mmol, 1.0 eq) and 6-bromohexan-1-ol (SM-5, 7.81 g, 43.38 mmol, 5 eq) in ethanol (40 mL) and water (40 mL) was added NaHCO₃ (7.37 g, 86.76 mmol, 10 eq) at room temperature. The resultant reaction mixture was heated to 80° C. and stirred for 16 h. Progress of the reaction was monitored by TLC. After complete consumption of starting materials, the reaction mixture was filtered through a pad of celite bed and washed with ethyl acetate (40 mL). The obtained filtrate was concentrated under reduced pressure and diluted with water (120 mL) and extracted with ethyl acetate (2×200 mL). The combined organic extracts were washed with brine (100 mL) and dried over anhydrous sodium sulphate, filtered, and concentrated under reduced pressure to obtain crude compound, which was purified by combi-flash chromatography by eluting with 70% ethyl acetate in heptane to afford (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthrene-17-yl acetate (Int-8, 2.6 g, 53%) as an off-white solid.

### Step-9: Synthesis of (8S,11R,13S,14S,17R)-17-acetyl-13-methyl-11-(4-(methyl(6-oxohexyl) amino)phenyl)-3-oxo-2,3,6,7,8,11,12,13,14,15,16,17-dodecahydro-1H-cyclopenta[a]phenanthren-17-yl acetate (Int-9)

To a stirred solution of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthrene-17-yl acetate (Int-8, 500 mg, 0.891 mmol, 1 eq) in ethyl acetate (40 mL), was added Dess-Martin periodinane (DMP, 1.1 g, 2.67 mmol, 3 eq), portion wise at 0° C. The resultant reaction mixture was heated to 80° C. and stirred for 2 h. Progress of the reaction was monitored by TLC. After complete consumption of starting materials, the reaction mixture was quenched with 50% aqueous Na₂S₂O₃ solution (10 mL), sat. NaHCO₃ solution (15 mL) and extracted with ethyl acetate (2×25 mL). The combined organic extracts were washed with brine (100 mL) and dried over anhydrous sodium sulphate, filtered, and concentrated under reduced pressure to afford (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-9, 450 mg, 92%) as a brown solid.

### Step-10: Synthesis of tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valinate (Int-10)

To a stirred solution of tert-butyl L-valinate (SM-6, 1.23 g, 7.15 mmol) in MeOH (20 mL) were added (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-9, 2 g, 3.57 mmol) and AcOH (0.2 mL) at 0° C. and warmed the reaction mixture to room temperature, stirred for 10 min. After that NaCNBH₃ (450 mg, 7.15 mmol) was added at 0° C. The resultant reaction mixture was stirred at room temperature for 2 h Progress of the reaction was monitored by TLC. After complete consumption of starting materials, the reaction mixture was cooled to 0° C. and quenched with sat. NaHCO₃ solution (100 mL), extracted with 10% McOH in DCM (2×100 mL). The combined organic layer was washed with water (50 mL), brine (50 mL) and dried over anhydrous Na₂SO₄, filtered and concentrated under reduced pressure to obtain crude product, which was washed with diethyl ether (20 mL) and dried under vacuum to afford tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valinate (Int-10, 1.2 g, 6.4%) as a brown solid.

### Step-11: Synthesis of (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valine (Int-6)

To a stirred solution of tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl) (methyl)amino)hexyl)-L-valinate (Int-10, 1.2 g, 1.67 mmol, 1.0 eq) in DCM (12 mL) under nitrogen atmosphere was added TFA (12 mL, 10 vol) at 0° C. The resultant reaction mixture was warmed to room temperature and stirred for 16 h. Progress of the reaction was monitored by TLC. After complete consumption of starting materials, the volatiles were evaporated under reduced pressure to obtain crude compound, which was washed with diethyl ether (20 mL) and dried under vacuum to afford (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valine (Int-6, 900 mg, crude) as a brown solid.

### Example S40. Preparation of N-((1r,4r)-4-((3-chloro-4-cyanophenyl)(methyl)amino)cyclohexyl)-6-(4-((4-(((S)-4-ethyl-4-hydroxy-9-methoxy-3,14-dioxo-3,4,12,14-tetrahydro-1H-pyrano[3′,4′:6,7]indolizino[1,2-b]quinolin-10-yl)methyl)piperazin-1-yl)methyl)piperidin-1-yl)pyridazine-3-carboxamide formic acid salt (Compound No. 40)

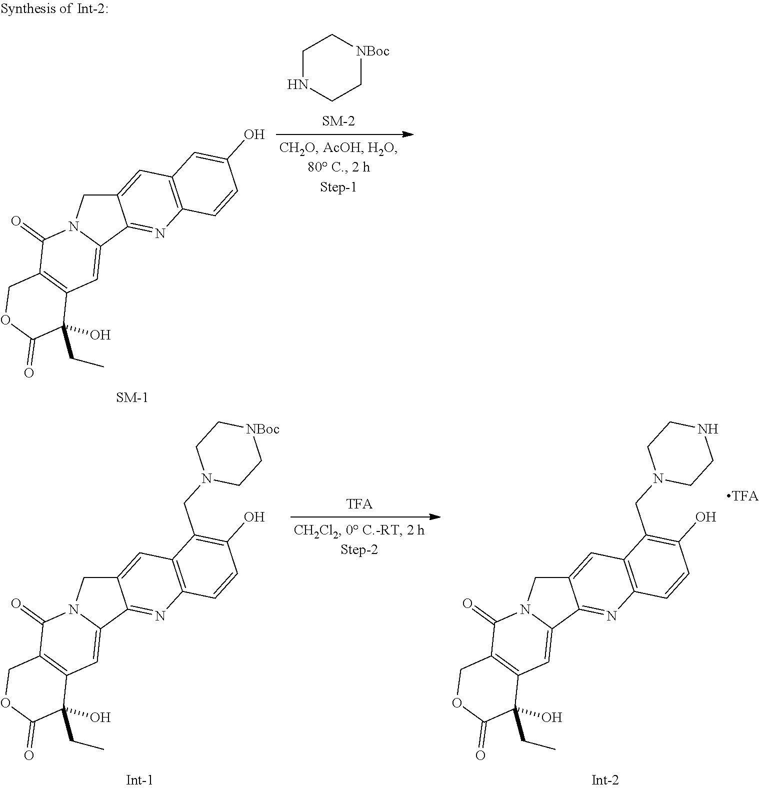 [SMILES: CC(C)(C)OC(=O)N1CCNCC1 ; CC[C@@]1(O)C(=O)OCc2c1cc1n(c2=O)Cc2cc3cc(O)ccc3nc2-1 ; CC[C@@]1(O)C(=O)OCc2c1cc1n(c2=O)Cc2cc3c(CN4CCN(C(=O)OC(C)(C)C)CC4)c(O)ccc3nc2-1 ; CC[C@@]1(O)C(=O)OCc2c1cc1n(c2=O)Cc2cc3c(CN4CCNCC4)c(O)ccc3nc2-1]

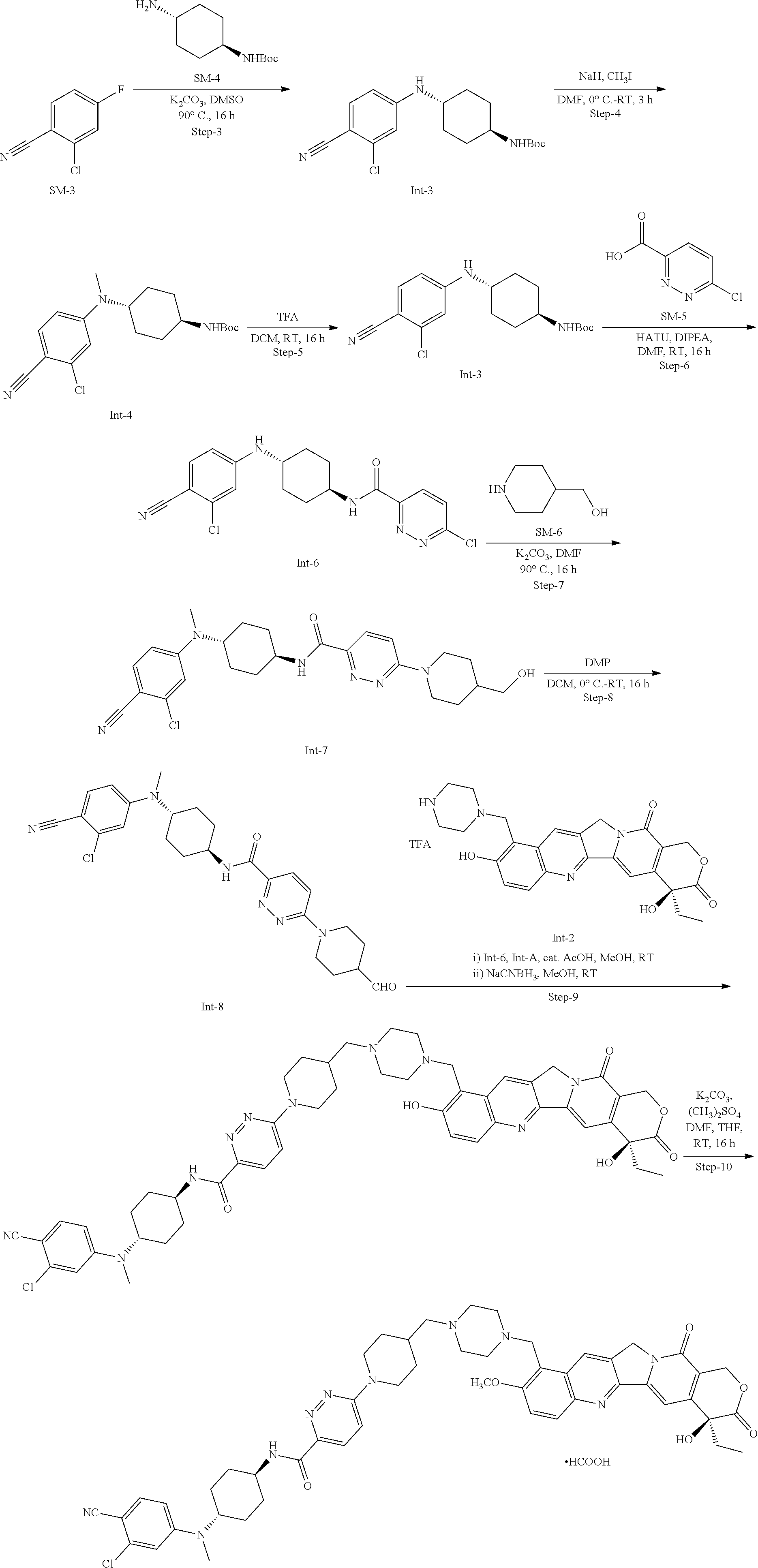

### Step-1: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-1)

To a stirred solution of (S)-4-ethyl-4,9-dihydroxy-1,12-dihydro-14H-pyrano[3′,4′:6,7]indolizino[1,2-b]quinoline-3,14(4H)-dione·HCl salt SM-1 (2.5 g, 6.86 mmol, 1.0 eq) and tert-butyl piperazine-1-carboxylate SM-2 (1.85 g, 10.3 mmol, 1.5 eq) in acetic acid (10 mL) under inert atmosphere was added 37% solution of formaldehyde (0.29 mL, 8.24 mmol, 1.2 eq) at room temperature. The reaction mixture was heated to 80° C. and stirred for 2 h in a sealed tube. Progress of the reaction was monitored by TLC. After completion of the reaction, solvents were evaporated under reduced pressure to get crude compound, the crude compound was basified with aq. ammonia until the pH reached to 9, filtered the solid and washed with water (10 mL) and dried under vacuum to afford Int-1 (1.9 g, 50%) as a yellow solid.

### Step-2: Synthesis of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFA salt (Int-2)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1 carboxylate Int-3 (2 g, 3.5 mmol, 1.0 eq) in DCM (20 mL) under nitrogen atmosphere was added TFA (2.7 mL, 35 mmol, 10 eq) at 0° C. Warmed to room temperature and stirred for 2 h. Progress of the reaction was monitored by TLC. After completion of the reaction, solvents were evaporated under reduced pressure, washed with diethyl ether (10 mL) and dried under vacuum to afford Int-2 (1.4 g, 83%) as a yellow solid.

### Step-3: Synthesis of tert-butyl ((1r,4r)-4-((3-chloro-4-cyanophenyl)amino) cyclohexyl)carbamate (Int-3)

To a stirred solution of 2-chloro-4-fluorobenzonitrile SM-3 (4 g, 25 mmol, 1.0 eq) in DMSO (40 mL) were added tert-butyl ((1r,4r)-4-aminocyclohexyl)carbamate SM-4 (5.5 g, 25 mmol, 1.0 eq) and K₂CO₃ (7.1 g, 51 mmol, 2 eq) at room temperature. The reaction mixture was heated to 90° C. for 16 h. Progress of the reaction was monitored by TLC. After completion of the reaction, the reaction mixture was diluted with ice cold water (200 mL) and extracted with ethyl acetate (2×400 mL). The combined organic extracts were washed with water (200 mL), brine (200 mL) and dried over anhydrous sodium sulphate, filtered, and concentrated under vacuum to obtain crude compound which was purified by combiflash column by eluting with 64% ethyl acetate in heptane to afford Int-3 (7.1 g, 78%) as an off white solid.

### Step-4: Synthesis of tert-butyl ((1r,4r)-4-((3-chloro-4-cyanophenyl)(methyl)amino)cyclohexyl)carbamate (Int-4)

To a stirred solution of tert-butyl ((1r,4r)-4-((3-chloro-4-cyanophenyl)amino)cyclohexyl) carbamate Int-3 (5.5 g, 15 mmol, 1.0 eq) in DMF (25 mL) under nitrogen atmosphere was added 60% of NaH (500 mg, 21 mmol, 1.3 eq) portion wise at 0° C. The reaction mixture was allowed to room temperature and stirred for 30 min then added methyl iodide (1.1 mL, 21 mmol, 1.3 eq) drop wise at 0° C. Reaction mixture was allowed to room temperature and stirred for 3 h. Progress of the reaction was monitored by TLC. After completion of the reaction, water (150 mL) was added and extracted with ethyl acetate (2×200 mL). Solvents were evaporated under reduced pressure to get crude compound which was purified by combiflash column by eluting with 15% ethyl acetate in heptane in to afford Int-4 (3.2 g, 55%) as an off white solid.

### Step-5: Synthesis of 4-(((1r,4r)-4-aminocyclohexyl)(methyl)amino)-2-chlorobenzonitrile. TFA Salt (Int-5)

To a stirred solution of tert-butyl ((1r,4r)-4-((3-chloro-4-cyanophenyl)(methyl)amino) cyclohexyl)carbamate Int-4 (1.1 g, 3 mmol, 1.0 eq) in DCM (10 mL) under nitrogen atmosphere was added TFA (10 mL, 10 vol) at 0° C. The reaction mixture was allowed to room temperature and stirred for 16 h. Progress of the reaction was monitored by TLC. After completion of the reaction, solvents were evaporated under reduced pressure, washed with diethyl ether (20 mL) and dried under vacuum to afford Int-5 (852 mg, 74%) as an off white solid.

### Step-6: Synthesis of 6-chloro-N-((1r,4r)-4-((3-chloro-4-cyanophenyl)(methyl)amino)cyclohexyl)pyridazine-3-carboxamide (Int-6)

To a stirred solution of 4-(((1r,4r)-4-aminocyclohexyl)(methyl)amino)-2-chlorobenzonitrile. TFA salt Int-5 (800 mg, 8 mmol, 1.0 eq) and 6-chloropyridazine-3-carboxylic acid SM-5 (336 mg, 8 mmol, 1.0 eq) in DMF (3 mL) were added HATU (1.21 g, 12 mmol, 1.5 eq) and DIPEA (0.74 mL, 16 mmol, 2 eq) at room temperature and stirred for 16 h. Progress of the reaction was monitored by TLC. After completion of the reaction, water (50 mL) was added and extracted with ethyl acetate (2×100 mL). The combined organic extracts were washed with water (100 mL), brine (100 mL) and dried over anhydrous sodium sulphate. Solvents were evaporated under reduced pressure to give crude compound which was purified by combiflash column by eluting with 4% methanol in DCM to afford Int-6 (700 mg, 81%) as an off white solid.

### Step-7: Synthesis of N-((1r,4r)-4-((3-chloro-4-cyanophenyl)(methyl)amino)cyclohexyl)-6-(4-(hydroxy methyl)piperidin-1-yl)pyridazine-3-carboxamide (Int-7)

To a stirred solution of Int-6 (2.0 g, 5 mmol, 1.0 eq) in DMF (10 mL) was added piperidin-4-ylmethanol SM-6 (0.690 g, 6 mmol, 1.2 eq) and K₂CO₃ (1.1 g, 8 mmol, 1.6 eq) at room temperature. The reaction mixture was heated to 90° C. and stirred for 16 h. Progress of the reaction was monitored by TLC. After completion of starting material, the reaction mixture was diluted with cold water (50 mL) and extracted with ethyl acetate (2×150 mL). The combined organic layers were washed with water (100 mL), brine (100 mL) and dried over anhydrous sodium sulphate, filtered, and concentrated under vacuum to obtain crude compound which was purified by combi flash column by eluting with 100% ethyl acetate to afford Int-7 (1.3 g, 54%) as an off white solid.

### Step-8: Synthesis of N-((1r,4r)-4-((3-chloro-4-cyanophenyl)(methyl)amino)cyclohexyl)-6-(4-formyl piperidin-1-yl)pyridazine-3-carboxamide (Int-8)

To a stirred solution of N-((1r,4r)-4-((3-chloro-4-cyanophenyl)(methyl)amino)cyclohexyl)-6-(4-(hydroxymethyl)piperidin-1-yl)pyridazine-3-carboxamide Int-7 (350 mg, 0.72 mmol, 1.0 eq) in DCM (6 mL) under nitrogen atmosphere was added Dess-martin periodinane (400 mg, 0.92 mmol, 1.3 eq) portion wise at 0° C. The reaction mixture was allowed to room temperature and stirred for 16 h. Progress of the reaction was monitored by TLC. After completion of the reaction, water (50 mL) was added and extracted with DCM (2×50 mL). The combined organic extracts were washed with sat. NaHCO₃ (50 mL) and sat. sodium thiosulphate (50 mL) solution, dried over anhydrous sodium sulphate, filtered and concentrated under reduced pressure to give crude Int-8 (320 mg, 91%) as an off-white solid which was used in next step without purification.

### Step-9: N-((1r,4r)-4-((3-chloro-4-cyanophenyl)(methyl)amino)cyclohexyl)-6-(4-((4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)methyl)piperidin-1-yl)pyridazine-3-carboxamide

To a stirred solution of N-((1r,4r)-4-((3-chloro-4-cyanophenyl)(methyl)amino)cyclohexyl)-6-(4-formylpiperidin-1-yl)pyridazine-3-carboxamide Int-8 (500 mg, 1.03 mmol, 1.0 eq) in methanol (5 mL) were added(S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFA salt Int-2 (622 mg, 1.35 mmol, 1.3 eq), triethyl amine (0.14 mL, 1.03 mmol, 1 eq) and acetic acid (0.2 mL) at room temperature and stirred for 2 h. To this reaction mixture added NaCNBH₃ (97 mg, 1.55 mmol, 1.5 eq) portion wise at 0° C. The reaction mixture was allowed to room temperature and stirred for 16 h. Progress of the reaction was monitored by TLC. After completion of the reaction, cold water (50 mL) was added and extracted with 20% MeOH in DCM (2×50 mL). The combined organic extracts were washed with water (60 mL), brine (50 mL) and dried over anhydrous sodium sulphate, filtered, and concentrated under vacuum to obtain crude compound which was purified by prep. HPLC purification method by eluting with Mobile phase A: 0.1% FA in water and Mobile phase B: Acetonitrile to afford the title compound (165 mg, 17%) as a yellow colour solid.

### Step-10: N-((1r,4r)-4-((3-chloro-4-cyanophenyl)(methyl)amino)cyclohexyl)-6-(4-((4-(((S)-4-ethyl-4-hydroxy-9-methoxy-3,14-dioxo-3,4,12,14-tetrahydro-1H-pyrano[3′,4′:6,7]indolizino[1,2-b]quinolin-10-yl)methyl)piperazin-1-yl)methyl)piperidin-1-yl)pyridazine-3-carboxamide formic acid salt

To a stirred solution of N-((1r,4r)-4-((3-chloro-4-cyanophenyl)(methyl)amino)cyclohexyl)-6-(4-((4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)methyl)piperidin-1-yl)pyridazine-3-carboxamide (300 mg, 0.32 mmol, 1 eq) in DMF (20 mL) and THF (20 mL) was added K₂CO₃ (89 mg, 0.65 mmol, 2 eq) and cooled to 0° C. followed by addition of dimethyl sulfate (0.06 mL, 0.65 mmol, 2 eq). Reaction mixture was allowed to stir at room temperature for 16 h. Progress of the reaction was monitored by TLC. After completion of the reaction, cold water (50 mL) was added and extracted with DCM (2×50 mL). The combined organic extracts were washed with water (60 mL), brine (50 mL) and dried over anhydrous sodium sulphate, filtered, and concentrated under vacuum to obtain crude compound which was purified by prep. HPLC purification method by eluting with Mobile phase A: 0.1% FA in water and Mobile phase B: Acetonitrile to afford the title compound (50 mg, 16%) as an off white solid.

¹H NMR (400 MHz, DMSO-d₆) δ ppm 8.84 (s, 1H), 8.45 (d, J=8.31 Hz, 1H), 8.12-8.17 (m, 2H), 7.79 (dd, J=9.54, 2.20 Hz, 2H), 7.60 (d, J=8.80 Hz, 1H), 7.27-7.34 (m, 2H), 6.94 (s, 1H), 6.83 (dd, J=8.80, 1.96 Hz, 1H), 6.48 (s, 1H), 5.42 (s, 2H), 5.28 (s, 2H), 4.45 (d, J=12.72 Hz, 2H), 3.99 (s, 3H), 3.93 (s, 2H), 3.82 (dd, J=19.81, 8.56 Hz, 3H), 2.99 (t, J=12.23 Hz, 2H), 2.85 (s, 3H), 2.33 (s, 4H), 2.11 (d, J=5.87 Hz, 2H), 1.88 (td, J=14.06, 7.58 Hz, 5H), 1.59-1.83 (m, 10H), 1.08 (d, J=11.74 Hz, 2H), 0.89 (t, J=7.09 Hz, 3H); LCMS: 99.66%; ESI MS m/z calcd. For C₅₁H₅₇ClN₁₀O₆ ([M+H]⁺) 941.53; found 941.4; HPLC: 98.47%.

### Example S41. Preparation of 4-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)butyl 4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Compound No. 41)

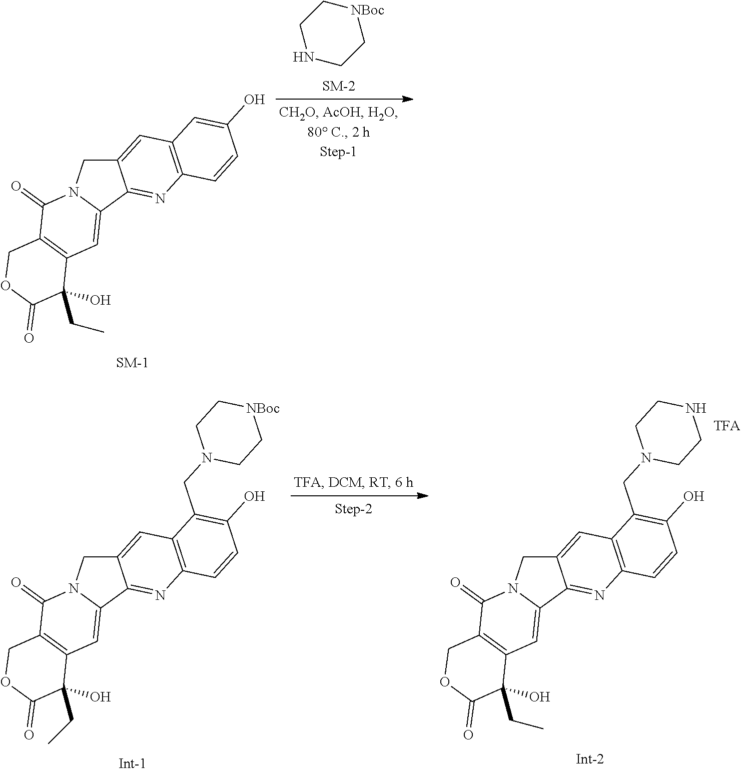 [SMILES: CC(C)(C)OC(=O)N1CCNCC1 ; CC[C@@]1(O)C(=O)OCc2c1cc1n(c2=O)Cc2cc3cc(O)ccc3nc2-1 ; CC[C@@]1(O)C(=O)OCc2c1cc1n(c2=O)Cc2cc3c(CN4CCN(C(=O)OC(C)(C)C)CC4)c(O)ccc3nc2-1 ; CC[C@@]1(O)C(=O)OCc2c1cc1n(c2=O)Cc2cc3c(CN4CCNCC4)c(O)ccc3nc2-1]

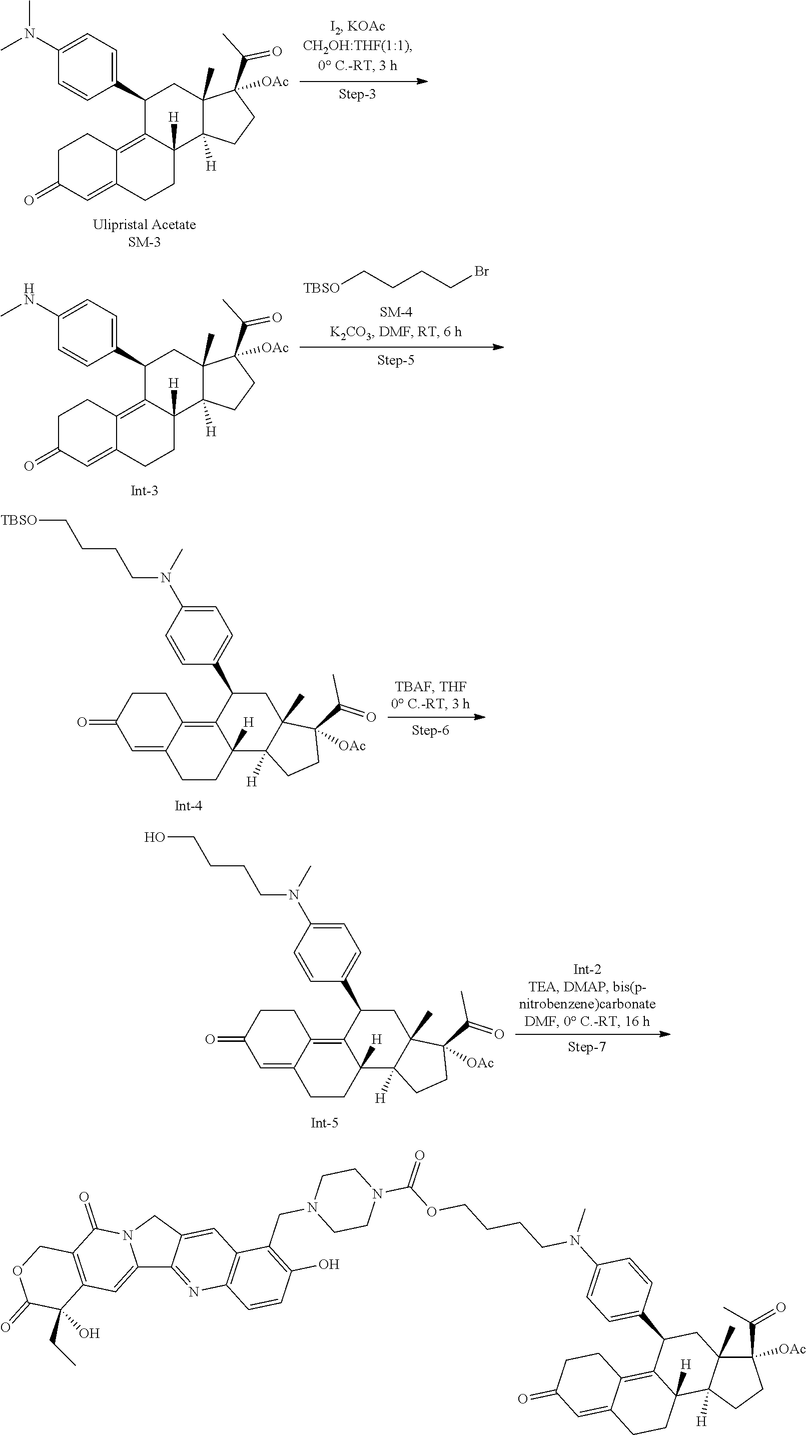

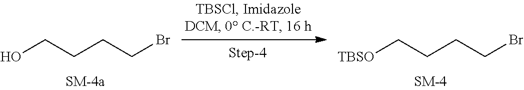 [SMILES: OCCCCBr ; CC(C)(C)[Si](C)(C)OCCCCBr]

### Step-1: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-1)

To a stirred solution of (S)-4-ethyl-4,9-dihydroxy-1,12-dihydro-14H-pyrano[3′,4′:6,7]indolizino[1,2-b]quinoline-3,14(4H)-dione·HCl salt (SM-1, 2.5 g, 6.86 mmol, 1.0 eq) and tert-butyl piperazine-1-carboxylate (SM-2, 1.85 g, 10.3 mmol, 1.5 eq) in acetic acid (10 mL) under inert atmosphere was added 37% solution of formaldehyde (0.29 mL, 8.24 mmol, 1.2 eq) at room temperature. The resultant reaction mixture was heated to 80° C. and stirred for 2 h in a sealed tube. Progress of the reaction was monitored by TLC. After complete consumption of starting materials, the volatiles were evaporated under reduced pressure to get crude compound, which was basified with aq. ammonia until the pH reached to 9 and filtered the solid, washed with water (10 mL) and dried under vacuum to afford Int-1 (1.9 g, 50%) as a yellow solid.

### Step-2: Synthesis of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFA salt (Int-2)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1 carboxylate (Int-1, 2 g, 3.5 mmol, 1.0 eq) in DCM (20 mL) under nitrogen atmosphere was added TFA (2.7 mL, 35 mmol, 10 eq) at 0° C. Warmed to room temperature and stirred for 2 h. Progress of the reaction was monitored by TLC. After complete consumption of the starting materials, the volatiles were evaporated under reduced pressure and the obtained crude compound was washed with diethyl ether (10 mL) and dried under vacuum to afford(S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFA salt (Int-2) (1.4 g, 83%) as a yellow solid.

### Step-3: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-3)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-3, 10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The resultant reaction mixture was allowed to room temperature and stirred for 3 h. Progress of the reaction was monitored by TLC. After complete consumption of the starting materials, the reaction mixture was quenched with sodium thiosulfate (Na₂S₂O₃) solution (50 g in 30 mL water) and extracted with ethyl acetate (2×200 mL), The combined organic extracts were washed with brine (100 mL) and dried over anhydrous sodium sulphate, filtered and concentrated under vacuum to afford (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-3, 8.0 g, 82%) as an off-white solid which was used in next step without further purification.

### Step-4: Synthesis of 4-bromobutoxy-tert-butyl-dimethyl-silane (SM-4)

To a stirred solution of 4-bromobutan-1-ol (SM-4a, 10 g, 26.14 mmol, 1.0 eq) in DCM (100 mL, 10 vol) TBDMS Chloride (11.76 g, 31.37 mmol, 1.2 eq) and Imidizole (8.8 g, 52.28 mmol, 2.0 eq) was added at 0° C. under argon atmosphere. Resulting reaction mixture was stirred at RT for 16 h TLC indicated complete consumption of starting material. After completion of the reaction, the reaction mixture was diluted with ice cold water (250 mL) and extracted with DCM (2×500 mL). The combined organic layer was washed with brine solution (200 mL), dried over anhydrous sodium sulphate, filtered, and concentrated under reduced pressure to obtain crude compound, which was purified by flash column (silica gel. 100-200 mesh) elution with 0-10% EtOAc in Hexane. Pure fractions were evaporated under reduced pressure to afford 4-bromobutoxy-tert-butyl-dimethyl-silane (SM-4, 5 g, 30%) as a colourless liquid.

### Step-5: Synthesis of [(8S,11R,13S,14S,17R)-17-acetyl-11-[4-[4-[tert-butyl(dimethyl)silyl]oxybutyl-methyl-amino]phenyl]-13-methyl-3-oxo-1,2,6,7,8,11,12,14, 15,16-decahydrocyclopenta[a]phenanthren-17-yl]acetate (Int-4)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-3, 3.0 g, 6.50 mmol, 1.0 eq) in DMF (30 mL, 10 vol), 4-bromobutoxy-tert-butyl-dimethyl-silane (SM-4, 3.48 g, 13.05 mmol, 2.0 eq) and K₂CO₃ (4.5 g, 32.5 mmol, 5.0 eq) were added at 0° C. under argon atmosphere. The resultant reaction mixture was stirred at RT for 6 h. Progress of the reaction was monitored by TLC, after complete consumption of the starting materials, the reaction mixture was diluted with water (50 mL) and extracted with ethyl acetate (2×100 mL). The combined organic layer was washed with brine solution (200 mL), dried over anhydrous sodium sulphate, filtered, and concentrated under reduced pressure to obtain crude compound, which was purified by flash column (silica gel. 100-200 mesh) eluted with 20-35% of EtOAc in Hexane. Pure fractions were evaporated under reduced pressure to obtain [(8S,11R,13S,14S,17R)-17-acetyl-11-[4-[4-[tert-butyl(dimethyl)silyl]oxybutyl-methyl-amino]phenyl]-13-methyl-3-oxo-1,2,6,7,8,11,12,14,15,16-decahydrocyclopenta[a]phenanthren-17-yl]acetate (Int-4, 1.0 g, 24%) as an orange gummy.

### Step-6: Synthesis of [(8S,11R,13S,14S,17R)-17-acetyl-11-[4-[4-hydroxybutyl(methyl)amino]phenyl]-13-methyl-3-oxo-1,2,6,7,8,11,12,14,15,16-decahydrocyclopenta[a]phenanthren-17-yl]acetate (Int-5)

To a stirred solution of [(8S,11R,13S,14S,17R)-17-acetyl-11-[4-[4-[tert-butyl(dimethyl)silyl]oxybutyl-methyl-amino]phenyl]-13-methyl-3-oxo-1,2,6,7,8,11,12,14,15,16-decahydrocyclopenta[a]phenanthren-17-yl]acetate (Int-4, 1.2 g, 1.85 mmol, 1.0 eq) in THF (10 mL) was added TBAF (3.6 mL, 3 vol) at 0° C. under argon atmosphere. The resultant reaction mixture was stirred at RT for 3 h. Progress of the reaction was monitored by TLC, after complete consumption of starting materials, the reaction mixture was diluted with water (50 mL) and extracted with ethyl acetate (2×100 mL). The combined organic layer was washed with brine solution (200 mL), dried over anhydrous sodium sulphate, filtered, and concentrated under reduced pressure to obtain crude compound, which was purified by flash column (silica gel. 100-200 mesh) elution with 40-50% EtOAc in Hexane. Pure fractions were evaporated under reduced pressure to afford [(8S,11R,13S,14S,17R)-17-acetyl-11-[4-[4-hydroxybutyl(methyl)amino]phenyl]-13-methyl-3-oxo-1,2,6,7,8,11,12,14,15,16-decahydrocyclopenta[a]phenanthren-17-yl]acetate (Int-5, 600 mg, 81%) as a white solid.

### Step-7: Synthesis of 4-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)butyl 4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate

To a stirred solution of [(8S,11R,13S,14S,17R)-17-acetyl-11-[4-[4-hydroxybutyl(methyl) amino]phenyl]-13-methyl-3-oxo-1,2,6,7,8,11,12,14,15,16-decahydrocyclopenta[a]phenanthren-17-yl] acetate (Int-5, 0.300 g, 0.562 mmol, 1.0 eq) in DCM (5 mL) were added Molecular sieves (300 mg) and Bis (4-Nitro phenyl) Carbonate (0.34 g, 1.125 mmol, 2 eq). Triethylamine (0.2 MI. 1.40 mmol, 2.5 eq) and DMAP (34 mg, 0.281 mmol, 0.5 eq) at RT and the resultant reaction mixture was stirred at RT for 3 h. followed by addition of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFA salt (Int-2, 0.26 g, 0562 mmol, 1.0 eq). Progress of the reaction was monitored by TLC, after complete consumption of starting materials, the reaction mixture was diluted with water (50 mL) and extracted with DCM (2×100 mL). The combined organic layer was washed with brine solution (200 mL), dried over anhydrous sodium sulphate, filtered, and concentrated under reduced pressure to obtain crude compound. Which was purified by Prep-HPLC to obtain the title compound (60 mg, 10%) as a white solid.

¹HNMR: ¹H NMR (400 MHz, DMSO-d₆) δ 8.76 (s, 1H), 7.99 (d, J=9.26 Hz, 1H), 7.47 (d, J=9.13 Hz, 1H), 7.26 (s, 1H), 6.96 (d, J=8.63 Hz, 2H), 6.46-6.62 (m, 2H), 5.65 (s, 1H), 5.41 (s, 2H), 5.23-5.27 (m, 2H), 4.33-4.38 (m, 1H), 4.01 (s, 4H), 3.21 (s, 4H), 2.81 (s, 2H), 2.65-2.77 (m, 4H), 2.34-2.39 (m, 2H), 2.02-2.24 (m, 9H), 1.79-2.01 (m, 10H), 1.62-1.75 (m, 3H), 1.45-1.60 (m, 5H), 1.22-1.42 (m, 4H), 0.88 (t, J=7.25 Hz, 3H), 0.19-0.25 (m, 3H).LCMS: 97.31%; ESI MS m/z calcd. For C₅₉H₆₇N₅O₁₁ ([M+H]+) 1022.21; found 1022.4; HPLC: 95.46%.

### Example S42. Preparation of 2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Compound No. 42)

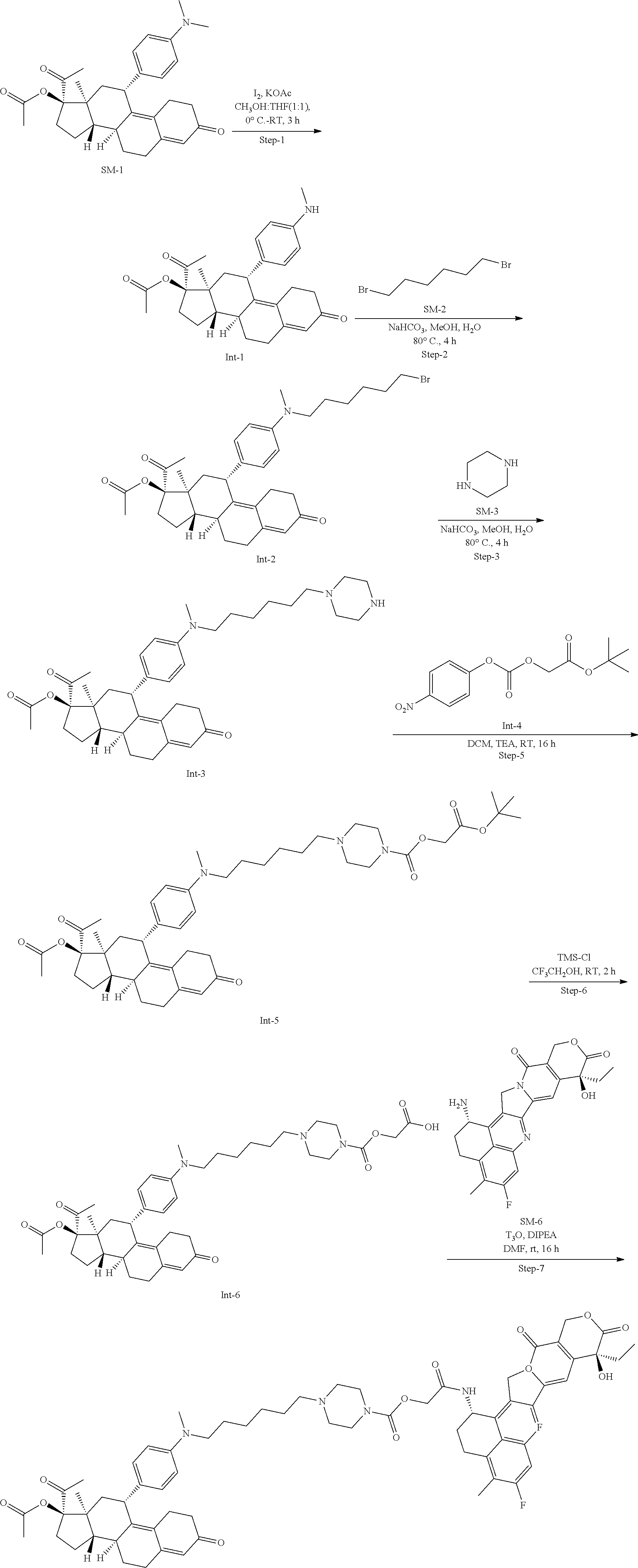

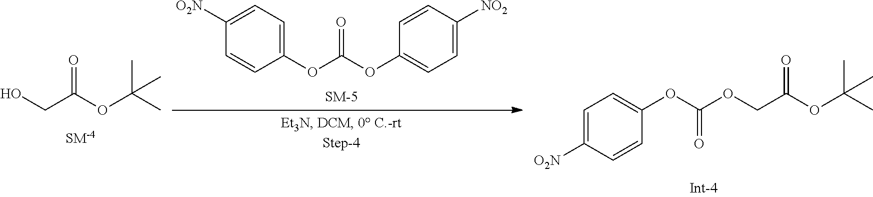

### Step-1: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-1, 15 g, 31.57 mmol, 1.0 eq) in methanol (75 mL) and THF (150 mL) were added KOAc (30.99 g, 315 mmol, 10 eq) and Iodine (20.33 g, 157 mmol, 5 eq) at 0° C. The resultant reaction mixture was allowed to room temperature and stirred for 3 h. Progress of the reaction was monitored by TLC. After complete consumption of starting materials, the reaction mixture was quenched with sodium thiosulfate (Na₂S₂O₃) solution (70 g in 45 mL water) and extracted with ethyl acetate (2×300 mL), The combined organic extracts were washed with brine (150 mL) and dried over anhydrous Na₂SO₄, filtered, and concentrated under vacuum to afford (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 9.5 g, 65%) as an off-white solid which was used in next step without further purification.

### Step-2: Synthesis of (8S,11R,13S,14S,17R)-17-acetyl-11-(4-((6-bromohexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2)

To a solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 8.5 g, 18.43 mmol, 1.0 eq) and 1,6-dibromohexane (SM-2, 22.47 g, 92.190 mmol, 5 eq) in methanol (85 mL) and water (28 mL) was added NaHCO₃ (4.6 g, 55.31 mmol, 3 eq) at room temperature. The resultant reaction mixture was heated to 80° C. and stirred for 1 h. Progress of the reaction was monitored by TLC. After complete consumption of the starting materials, the reaction mixture was filtered through a pad of celite bed and washed with ethyl acetate (85 mL). Filtrate was concentrated under reduced pressure, diluted with water (240 mL) and extracted with ethyl acetate (2×400 mL), The combined organic extracts were washed with brine (200 mL) and dried over anhydrous Na₂SO₄, filtered, and concentrated under vacuum to obtain crude compound, which was purified by combi flash chromatography by eluting with 70% ethyl acetate in heptane to afford (8S,11R,13S,14S,17R)-17-acetyl-11-(4-((6-bromohexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 4 g, 34%) as an off-white solid.

### Step-3: Synthesis of (8S,11R,13S,14S,17R)-17-acetyl-13-methyl-11-(4-(methyl(6-(piperazin-1-yl)hexyl)amino)phenyl)-3-oxo-2,3,6,7,8,11,12,13,14,15,16,17-dodecahydro-1H-cyclopenta[a]phenanthren-17-yl acetate (Int-3)

To a stirred solution of (8S,11R,13S,14S,17R)-17-acetyl-11-(4-((6-bromohexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 4.0 g, 6.410 mmol, 1.0 eq) and piperazine (SM-3, 2.75 g, 32.05 mmol, 5 eq) in methanol (80 mL) and water (26 mL) was added NaHCO₃ (1.61 g, 26.03 mmol, 3 eq) at room temperature. The resultant reaction mixture was heated to 80° C. and stirred for 16 h. Progress of the reaction was monitored by TLC. After complete consumption of the starting materials, the reaction mixture was filtered through a pad of celite bed and washed with ethyl acetate (85 mL). Filtrate was minimized under reduced pressure and diluted with water (240 mL), extracted with ethyl acetate (2×400 mL). The combined organic extracts were washed with brine (200 mL), dried over anhydrous Na₂SO₄, filtered, and concentrated under reduced pressure to obtain crude compound, which was purified by combi flash chromatography by eluting with 70% ethyl acetate in heptane to afford (8S,11R,13S,14S,17R)-17-acetyl-13-methyl-11-(4-(methyl(6-(piperazin-1-yl)hexyl)amino)phenyl)-3-oxo-2,3,6,7,8,11,12,13,14,15,16,17-dodecahydro-1H-cyclopenta[a]phenanthren-17-yl acetate (Int-3, 1.2 g, 30%) as an off-white solid.

### Step-4: Synthesis of tert-butyl 2-(((4-nitrophenoxy)carbonyl)oxy)acetate (Int-4)

To a stirred solution of tert-butyl 2-hydroxyacetate (SM-4, 0.5 g, 3.78 mmol, 1.0 eq) and bis(4-nitrophenyl) carbonate (SM-5, 6.3 g, 4.16 mmol, 1.1 eq) in anhydrous DCM was added triethylamine at 0° C. The resultant reaction mixture was stirred at room temperature for 2 h. Progress of the reaction was monitored by TLC, after complete consumption of starting materials, the reaction mixture was quenched with water (10 mL) and extracted with DCM (2×20 mL). The combined organic layer was washed with brine solution (10 mL) and dried over anhydrous Na₂SO₄. Filtered, and concentrated under reduced pressure to obtain tert-butyl 2-(((4-nitrophenoxy)carbonyl)oxy)acetate (Int-4, 1 g crude) as a yellow solid. The obtained material was used as such in next step without any further purification and analytical data.

### Step-5: Synthesis of 2-(tert-butoxy)-2-oxoethyl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Int-5)

A flask was charged with (8S,11R,13S,14S,17R)-17-acetyl-13-methyl-11-(4-(methyl(6-(piperazin-1-yl)hexyl)amino)phenyl)-3-oxo-2,3,6,7,8,11,12,13,14,15,16,17-dodecahydro-1H-cyclopenta[a]phenanthren-17-yl acetate (Int-3, 1.2 g, 1.90 mmol, 1.0 eq) and TEA (500 mg, 5.71 mmol, 3.0 eq) in dichloromethane was added tert-butyl 2-(((4-nitrophenoxy)carbonyl)oxy)acetate (Int-4, 1 g, 2.09 mmol, 1.1 eq) in DCM (10 mL, 10 vol). The resultant reaction mixture was stirred under nitrogen atmosphere at room temperature for 16 h. Progress of the reaction was monitored by TLC, after complete consumption of starting materials, the reaction mixture was diluted with water (100 mL) and extracted with DCM (2×200 mL). The combined organic layer was washed with brine solution (100 mL) and dried over anhydrous Na₂SO₄. Filtered, and concentrated under reduced pressure to obtain crude compound, which was purified by column chromatography by eluting with 1% Methanol in DCM to afford 2-(tert-butoxy)-2-oxoethyl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Int-5, 700 mg, 46%) as a white solid.

### Step-6: Synthesis of 2-((4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carbonyl)oxy)acetic acid (Int-6)

To a stirred solution of 2-(tert-butoxy)-2-oxoethyl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Int-5, 700 mg, 0.88 mmol, 1 eq) in trifluoro ethanol (11.6 mL) was added chlorotrimethylsilane (1.93 g, 17.7 mmol, 20 eq) drop wise at 0° C. The resultant reaction mixture was allowed to room temperature and stirred for 2 h. Progress of the reaction was monitored by TLC. After complete consumption of the starting materials, the organic solvents were evaporated under reduced pressure to obtain crude compound, which was further triturated with pentane (2×20 mL) and dried under vacuum to afford 2-((4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carbonyl)oxy)acetic acid (Int-6, 600 mg, crude) as a pale-yellow solid.

### Step-7: Synthesis of 2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate

To a stirred solution of 2-((4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carbonyl)oxy)acetic acid (Int-6, 0.6 g, 0.82 mmol, 1 eq) in 6 mL of anhydrous DMF, T₃P (0.52 g, 1.64 mmol, 2 eq) DIPEA (3 eq) and followed by (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (SM-6, 0.43 g, 0.82 mmol, 1 eq) were added. The resultant reaction mixture was stirred at room temperature for 16 h. Progress of the reaction was monitored by TLC, after complete consumption of starting materials, the reaction mixture was poured into ice cold water and filtered the solid, washed with water and dried under vacuum, the obtained solid was purified by reverse phase column chromatography with C18 silica to get compound at 50% Acetonitrile in H₂O to afford the title compound (217 mg, 16.8%) as a white solid.

¹H NMR (400 MHz, DMSO-d₆) δ 8.54 (d, J=8.51 Hz, 1H), 8.24 (s, 1H), 7.80 (d, J=10.88 Hz, 1H), 7.31 (s, 1H), 6.97 (d, J=8.63 Hz, 2H), 6.49-6.60 (m, 3H), 5.66 (s, 1H), 5.56 (dt, J=8.32, 4.22 Hz, 1H), 5.42 (s, 2H), 5.15-5.31 (m, 2H), 4.36-4.49 (m, 3H), 3.18 (s, 4H), 2.81 (s, 3H), 2.70-2.79 (m, 2H), 2.54-2.63 (m, 2H), 2.41 (s, 3H), 2.31-2.34 (m, 1H), 2.15-2.28 (m, 9H), 2.13 (s, 2H), 2.10 (s, 4H), 1.99 (s, 4H), 1.79-1.95 (m, 5H), 1.62-1.74 (m, 4H), 1.32-1.50 (m, 6H), 1.26 (s, 4H), 0.87 (t, J=7.32 Hz, 3H), 0.23 (s, 3H), LCMS: 98.26%; ESI MS m/z calcd. For C₆₆H₇₇FN₆O₁₁ ([M+H]+) 1149.37; found 1149.71; HPLC: 97.94%.

### Example S43. Preparation of N-((1r,4r)-4-((3-chloro-4-isocyanophenyl)(methyl)amino)cyclohexyl)-6-(4-(6-(4-(((S)-4-ethyl-4-hydroxy-3,14-dioxo-3,4,12,14-tetrahydro-1H-pyrano[3′,4′:6,7]indolizino[1,2-b]quinolin-10-yl)methyl)piperazin-1-yl)-6-oxohexyl)piperidin-1-yl)pyridazine-3-carboxamide (Compound No. 43)

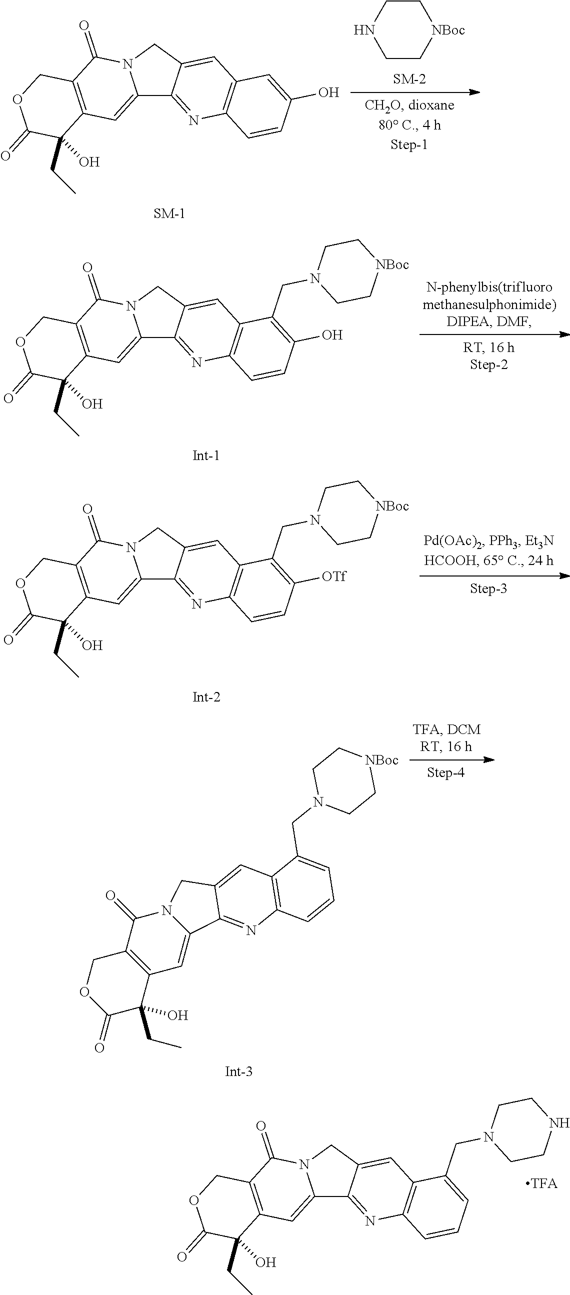 [SMILES: CC[C@@]1(O)C(=O)OCc2c1cc1n(c2=O)Cc2cc3cc(O)ccc3nc2-1 ; CC(C)(C)OC(=O)N1CCNCC1 ; CC[C@@]1(O)C(=O)OCc2c1cc1n(c2=O)Cc2cc3c(CN4CCN(C(=O)OC(C)(C)C)CC4)c(O)ccc3nc2-1 ; CC[C@@]1(O)C(=O)OCc2c1cc1n(c2=O)Cc2cc3c(CN4CCN(C(=O)OC(C)(C)C)CC4)c(OS(=O)(=O)C(F)(F)F)ccc3nc2-1 ; CC[C@@]1(O)C(=O)OCc2c1cc1n(c2=O)Cc2cc3c(CN4CCN(C(=O)OC(C)(C)C)CC4)cccc3nc2-1 ; CC[C@@]1(O)C(=O)OCc2c1cc1n(c2=O)Cc2cc3c(CN4CCNCC4)cccc3nc2-1]

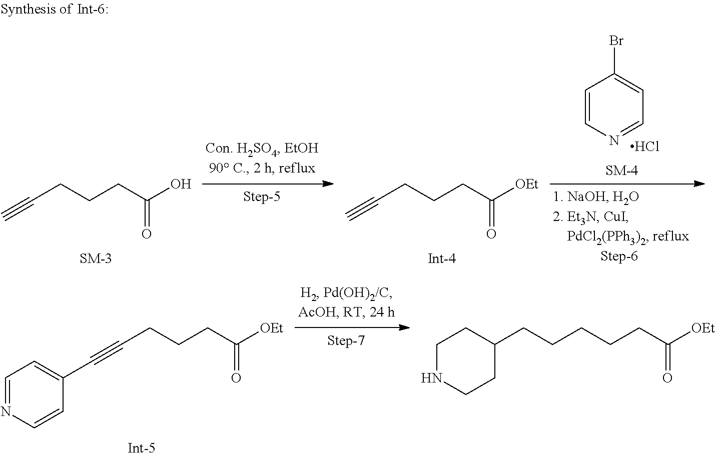

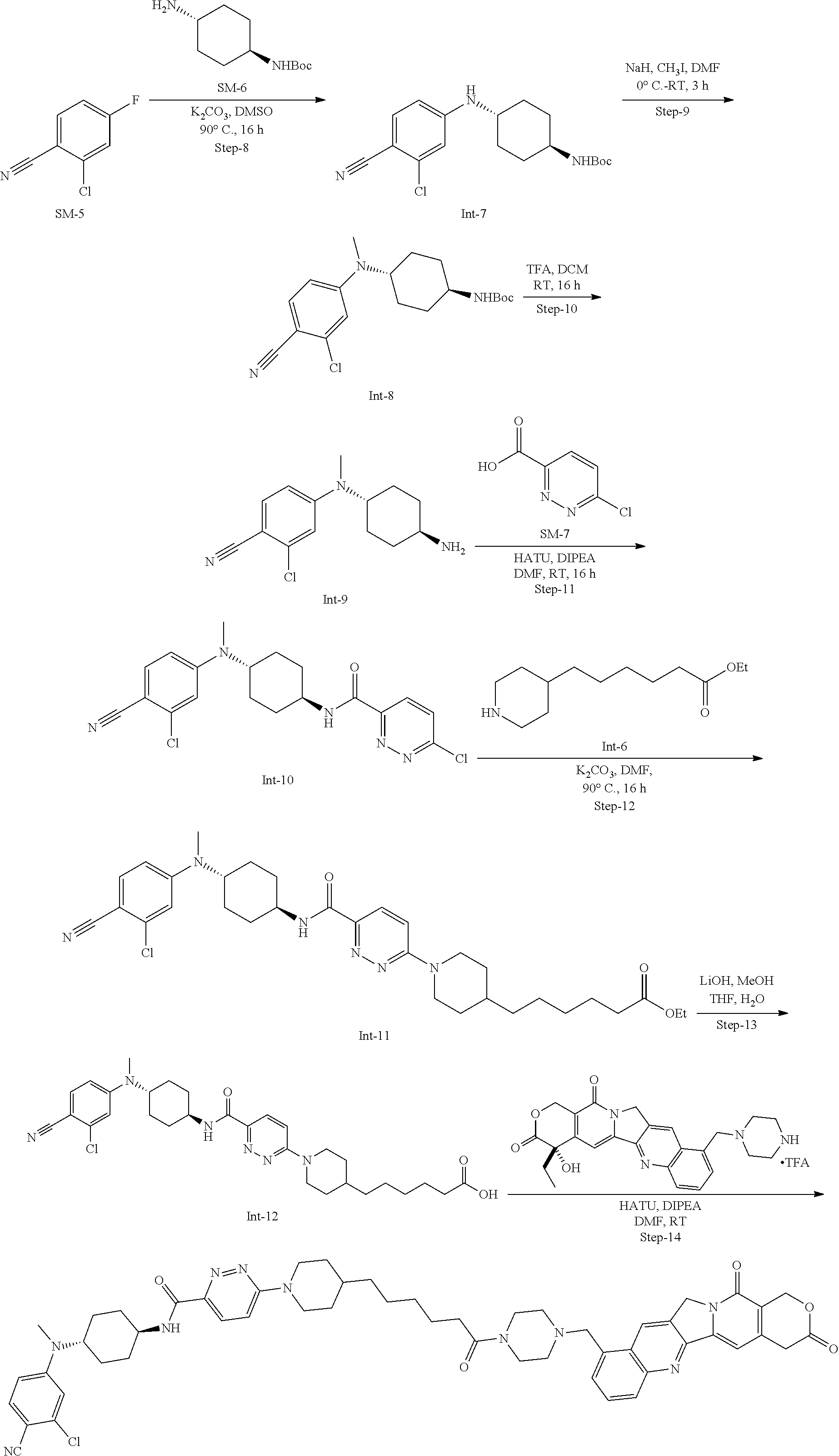

### Step-1: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-1)

A stirred solution of tert-butyl piperazine-1-carboxylate, SM-2 (3.8 g, 20.6 mmol, 1.5 eq) and aqueous formaldehyde solution (37-41%) (1.3 mL, 16.4 mmol, 1.2 eq) in 1,4-dioxane (50 mL) in a sealed tube was heated 80° C. for 1 h. Reaction mixture was cooled to room temperature, (S)-4-ethyl-4,9-dihydroxy-1,12-dihydro-14H-pyrano[3′,4′:6,7]indolizino[1,2-b]quinoline-3,14(4H)-dione, SM-1 (2.0 g, 5.49 mmol, 1 eq) was added portion wise over a period of 5 min and heating at 80° C. was continued for another 4 h. Progress of the reaction was monitored by TLC/LCMS. After completion, the reaction mixture was diluted with water (50 mL) and extracted with DCM (2×75 mL). Combined organic layers were washed with brine (50 mL), water (50 mL), dried over anhydrous Na₂SO₄ and concentrated under reduced pressure to give which was triturated with diethyl ether twice (2×50 mL) to afford Int-1 (4.8 g, 62%) as a yellow colour solid which was used for next step without further purification.

### Step-2: Synthesis of tert-butyl (S)-4-((4-ethyl-4-hydroxy-3,14-dioxo-9-(((trifluoromethyl) sulfonyl)oxy)-3,4,12,14-tetrahydro-1H-pyrano[3′,4′:6,7]indolizino[1,2-b]quinolin-10-yl)methyl)piperazine-1-carboxylate (Int-2)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate, Int-1 (2.0 g, 3.55 mmol, 1 eq), in DMF (20 mL) were added N-phenylbis (trifluoromethanesulphonimide) (1.9 g, 5.33 mmol, 1.5 eq) and DIPEA (1.85 mL, 10.67 mmol, 3 eq) dropwise over a period of 10 min. at 0° C. under nitrogen atmosphere and the reaction mixture was allowed to stir at room temperature for 16 h. Progress of the reaction was monitored by TLC/LCMS. After completion, the reaction mixture was quenched with water (50 mL) and extracted with 10% MeOH in DCM (2×75 mL). Combined organic layers were washed with brine (50 mL), water (50 mL), dried over anhydrous Na₂SO₄ and concentrated under reduced pressure to afford Int-2 (1.48 g, 61%) as an off-white solid.

### Step-3: Synthesis of tert-butyl (S)-4-((4-ethyl-4-hydroxy-3,14-dioxo-3,4,12,14-tetrahydro-1H-pyrano[3′,4′:6,7]indolizino[1,2-b]quinolin-10-yl)methyl)piperazine-1-carboxylate (Int-3)

To a stirred solution of tert-butyl (S)-4-((4-ethyl-4-hydroxy-3,14-dioxo-9-(((trifluoromethyl) sulfonyl)oxy)-3,4,12,14-tetrahydro-1H-pyrano[3′,4′:6,7]indolizino[1,2-b]quinolin-10-yl)methyl)piperazine-1-carboxylate, Int-2 (1.5 g, 2.16 mmol, 1 eq) in DMF (20 mL) were added triphenyl phosphine (56 mg, 0.21 mmol, 0.1 eq). Palladium (II) acetate (24 mg, 0.11 mmol, 0.05 eq) and triethyl amine (0.8 mL, 6.47 mmol, 3 eq) followed by dropwise addition of formic acid (0.34 mL, 8.6 mmol, 4 eq) over a period of 10 min and then the reaction mixture was heated to 65° C. and stirred for 24 h. Progress of the reaction was monitored by TLC/LCMS. After completion, the reaction mixture was quenched with water (50 mL) and extracted with 10% MeOH in DCM (2×75 mL). Combined organic layers were washed with brine (50 mL), water (50 mL), dried over anhydrous Na₂SO₄, filtered, and concentrated under reduced pressure to give crude product which was purified by combiflash column chromatography by eluting with 60% ethyl acetate in heptane to afford Int-3 (630 mg, 53%) as an off-white solid.

### Step-4: Synthesis of (S)-4-ethyl-4-hydroxy-10-(piperazin-1-ylmethyl)-1,12-dihydro-14H-pyrano[3′,4′:6,7]indolizino[1,2-b]quinoline-3,14(4H)-dione. CF₃COOH salt

A stirred solution of tert-butyl (S)-4-((4-ethyl-4-hydroxy-3,14-dioxo-3,4,12,14-tetrahydro-1H-pyrano[3′,4′:6,7]indolizino[1,2-b]quinolin-10-yl)methyl)piperazine-1-carboxylate, Int-3 (450 mg, 0.824 mmol, 1 eq) in dichloromethane (10 mL) was cooled to 0° C. under inert atmosphere followed by addition of trifluoroacetic acid (0.64 mL, 8.24 mmol, 10 eq) and the reaction mixture was warmed to room temperature and stirred for 16 h. Progress of the reaction was monitored by TLC. After complete consumption of Int-3, solvent was evaporated to obtain crude compound which was triturated with diethyl ether (2×25 mL) to afford the title compound (280 mg, 76%) as a yellow colour solid.

### Step-5: Synthesis of ethyl hex-5-ynoate (Int-4)

To a stirred solution of hex-5-ynoic acid (1.0 g, 8.92 mmol, 1 eq) in ethanol was added Con. H₂SO₄ at 0° C. and the resulting reaction mixture was heated to 90° C. and stirred for 2 h. Progress of the reaction was monitored by TLC and LCMS. After completion, reaction mixture was cooled to room temperature and quenched with sat. NaHCO₃ solution (2×10 mL) and extracted with EtOAc (2×25 mL). The combined organic layers were dried over anhydrous sodium sulphate, filtered and concentrated under reduced pressure to give crude Int-4 (1 g, crude) as pale-yellow liquid which was used as such for next step without any further purification.

### Step-6: Synthesis of ethyl 6-(pyridin-4-yl)hex-5-ynoate (Int-5)

A solution of 4-bromopyridine. HCl salt, SM-4 (1.0 g, 5.15 mmol, 1 eq) in EtOAc (250 mL) was cooled to 0° C. followed by addition of aqueous sodium hydroxide solution (2.06 g, 51.5 mmol, 10 eq) in water (30 mL) and then the reaction mixture was allowed to stir at room temperature for 30 minutes. After consumption of SM-4 as monitored by TLC, both the layers were separated, and organic layer was washed with brine (40 mL) solution, dried over anhydrous Na₂SO₄, filtered, and concentrated under reduced pressure to give 4-bromopyridine (1.0 g, crude) (HCl salt free) as colourless liquid.

To a stirred solution of above prepared 4-bromopyridine (1.0 g, 5.15 mmol, 1 eq) and ethyl hex-5-ynoate Int-4 (720 mg, 5.14 mmol, 1 eq) in triethyl amine (20 mL) were added copper iodide (19.6 mg, 0.103 mmol, 0.02 eq) and PdCl₂(PPh₃)₂ (39.6 mg, 0.056 mmol, 0.01 eq) at room temperature and the reaction mixture was heated to 80° C. and stirred for 1 h. Progress of the reaction was monitored by TLC/LCMS. After completion, the reaction mixture was diluted with water (50 mL) and extracted with EtOAc (2×80 mL). The combined organic layers were washed with brine (80 mL), water (75 mL), dried over anhydrous Na₂SO₄, filtered, and concentrated under reduced pressure to give crude Int-5 (1.0 g, 64%) as dark brown oil which was used as such for next step without any further purification.

### Step-7: Synthesis of ethyl 6-(piperidin-4-yl)hexanoate (Int-6)

To a solution of ethyl 6-(pyridin-4-yl)hex-5-ynoate, Int-5 (1 g, 4.6 mmol, 1 eq) in acetic acid (40 mL) was in a autoclave was added Pd(OH) 2/C (650 mg) and filled with H₂ gas up to 10 kg and reaction mixture was allowed to stir at ambient temperature for 24 h. Progress of the reaction was monitored by TLC. After completion, the reaction mixture was filtered through a celite bed and washed with EtOAc (100 mL). The combined filtrate was concentrated under reduced pressure to afford Int-6 (1.0 g, 96%) as a dark brown gum which was used as such for next step without any further purification. ¹H NMR (400 MHz, DMSO-d₆) δ ppm 3.98-4.11 (m, 2H) 3.15 (d, J=11.25 Hz, 1H) 2.71 (t, J=11.74 Hz, 1H) 2.26 (t, J=7.34 Hz, 2H) 1.69-1.76 (m, 4H) 1.36-1.77 (m, 4H) 1.06-1.25 (m, 11H); LCMS: 98.96%; ESI MS m/z calcd. For C₁₃H₂₅NO₂ ([M+H]⁺) 227.35; found 228.3.

### Step-8: Synthesis of tert-butyl ((1r,4r)-4-((3-chloro-4-cyanophenyl)amino)cyclohexyl)carbamate (Int-7)

To a stirred solution of 2-chloro-4-fluorobenzonitrile SM-5 (4 g, 25 mmol, 1.0 eq) in DMSO (40 mL) were added tert-butyl ((1r,4r)-4-aminocyclohexyl)carbamate SM-6 (5.5 g, 25 mmol, 1.0 eq) and K₂CO₃ (7.1 g, 51 mmol, 2 eq) at room temperature and the reaction mixture was heated to 90° C. for 16 h. Progress of the reaction was monitored by TLC. After completion, the reaction mixture was diluted with ice cold water (100 mL) and extracted with ethyl acetate (2×200 mL), The combined organic extracts were washed with water (100 mL), brine (100 mL) and dried over anhydrous sodium sulphate, filtered, and concentrated under vacuum to afford crude compound which was purified by Combiflash column by eluting with 64% ethyl acetate in heptane to afford Int-7 (7.1 g, 78%) as an off white solid.

### Step-9: Synthesis of tert-butyl ((1r,4r)-4-((3-chloro-4-cyanophenyl)(methyl)amino)cyclohexyl)carbamate (Int-8)

To a stirred solution of tert-butyl ((1r,4r)-4-((3-chloro-4-cyanophenyl)amino)cyclohexyl) carbamate Int-7 (5.5 g, 15 mmol, 1.0 eq) in DMF (25 mL) under nitrogen atmosphere was added 60% of NaH in paraffin oil (500 mg, 21 mmol, 1.3 eq) portion wise at 0° C. and the reaction mixture was allowed to stir at room temperature for 30 min and then methyl iodide (1.1 mL, 21 mmol, 1.3 eq) was added drop wise at 0° C. and stirred for 3 h at room temperature and. Progress of the reaction was monitored by TLC. After completion, the reaction mixture was cooled to 0° C., diluted with water (150 mL), extracted with ethyl acetate (2×150 mL). The combined organic extracts were dried over Na₂SO₄ and concentrated under reduced pressure to give crude compound which was purified by combiflash column by eluting with 15% ethyl acetate in heptane in to afford Int-8 (3.2 g, 55%) as an off-white solid.

### Step-10: Synthesis of 4-(((1r,4r)-4-aminocyclohexyl)(methyl)amino)-2-chlorobenzonitrile. TFA Salt (Int-9)

To a stirred solution of tert-butyl ((1r,4r)-4-((3-chloro-4-cyanophenyl)(methyl)amino) cyclohexyl)carbamate Int-8 (1.1 g, 3 mmol, 1.0 eq) in DCM (10 mL) under nitrogen atmosphere was added TFA (10 mL, 10 vol) at 0° C. and the reaction mixture was allowed to room temperature and stirred for 16 h. Progress of the reaction was monitored by TLC. After completion, the reaction mixture was concentrated under reduced pressure and the obtained residue was washed with diethyl ether (20 mL) and dried under vacuum to afford Int-9 (852 mg, 74%) as an off-white solid.

### Step-11: Synthesis of 6-chloro-N-((1r,4r)-4-((3-chloro-4-cyanophenyl)(methyl)amino)cyclohexyl)pyridazine-3-carboxamide (Int-10)

To a stirred solution of 4-(((1r,4r)-4-aminocyclohexyl)(methyl)amino)-2-chlorobenzonitrile. TFA salt Int-9 (800 mg, 8 mmol, 1.0 eq) and 6-chloropyridazine-3-carboxylic acid SM-7 (336 mg, 8 mmol, 1.0 eq) in DMF (3 mL) were added HATU (1.21 g, 12 mmol, 1.5 eq) and DIPEA (0.74 mL, 16 mmol, 2 eq) at room temperature and stirred for 16 h. Progress of the reaction was monitored by TLC. After completion, the reaction mixture was diluted with water (50 mL) and extracted with ethyl acetate (2×100 mL). The combined organic extracts were washed with water (100 mL), brine (100 mL) and dried over anhydrous sodium sulphate and evaporated under reduced pressure to give crude compound which was purified by combiflash column by eluting with 4% methanol in DCM to afford Int-10 (700 mg, 81%) as an off-white solid.

### Step-12: Synthesis of ethyl 6-(1-(6-(((1r,4r)-4-((3-chloro-4-cyanophenyl)(methyl)amino)cyclohexyl)carbamoyl)pyridazin-3-yl)piperidin-4-yl)hexanoate (Int-11)

To a stirred solution of 6-chloro-N-((1r,4r)-4-((3-chloro-4-cyanophenyl)(methyl)amino)cyclohexyl)pyridazine-3-carboxamide Int-10 (1.0 g, 2.5 mmol, 1 eq) and ethyl 6-(piperidin-4-yl)hexanoate, Int-6 (730 mg, 3.22 mmol, 1.3 eq) in acetonitrile (20 mL) were added potassium carbonate (1.25 g, 9.06 mmol, 3.7 eq) and KI (205 mg, 1.23 mmol, 0.5 eq) at room temperature and the reaction mixture was heated to 90° C. and stirred for 16 h. Progress of the reaction was monitored by TLC/LCMS. After completion, the reaction mixture was diluted with water (20 mL) and extracted with EtOAc (2×100 mL). The combined organic extracts were washed with water (50 mL) and brine (50 mL), dried over anhydrous sodium sulphate, filtered, and concentrated under vacuum to give crude compound which was purified by combiflash column chromatography by eluting with 30% ethyl acetate in n-heptane to afford Int-11 (480 mg, 33%) as an off-white solid.

### Step-13: Synthesis of 6-(1-(6-(((1r,4r)-4-((3-chloro-4-cyanophenyl)(methyl)amino)cyclohexyl)carbamoyl)pyridazin-3-yl)piperidin-4-yl)hexanoic Acid (Int-12)

To a stirred solution of 6-(1-(6-(((1r,4r)-4-((3-chloro-4-cyanophenyl)(methyl)amino)cyclohexyl)carbamoyl)pyridazin-3-yl)piperidin-4-yl)hexanoate. Int-11 (200 mg, 0.336 mmol, 1 eq) in MeOH (2 mL). THF (2 mL) and water (1 mL) was added lithium hydroxide (80 mg, 3.34 mmol, 10 eq) portion wise over a period of 5 min and the resulting reaction mixture was stirred at room temperature 16 h. Progress of the reaction was monitored by TLC and LCMS. After completion, solvents were evaporated under reduced pressure and crude material was acidified with 1N HCl solution (6 mL) (until P^{H}=3) and extracted with 10% MeOH in DCM (2×150 mL). The combined organic extracts were washed with water (15 mL), brine (15 mL) and dried over anhydrous sodium sulphate, filtered and concentrated under vacuum to afford Int-12 (180 mg, 94%) as an off-white solid which was carried for next step without any purification.

### Step-14: Synthesis of N-((1r,4r)-4-((3-chloro-4-isocyanophenyl)(methyl)amino)cyclohexyl)-6-(4-(6-(4-(((S)-4-ethyl-4-hydroxy-3,14-dioxo-3,4,12,14-tetrahydro-1H-pyrano[3′,4′:6,7]indolizino[1,2-b]quinolin-10-yl)methyl)piperazin-1-yl)-6-oxohexyl)piperidin-1-yl)pyridazine-3-carboxamide

To a stirred solution of 6-(1-(6-(((1r,4r)-4-((3-chloro-4-cyanophenyl)(methyl)amino)cyclohexyl)carbamoyl)pyridazin-3-yl)piperidin-4-yl)hexanoic acid, Int-12 (400 mg, 0.705 mmol, 1 eq) in DMF (5 mL) were added HATU (400 mg, 1.03 mmol, 1.5 eq) and DIPEA (0.37 mL, 2.10 mmol, 3 eq) under nitrogen atmosphere at room temperature and the resulting mixture stirred at room temperature for 5 min followed by addition of (S)-4-ethyl-4-hydroxy-10-(piperazin-1-ylmethyl)-1,12-dihydro-14H-pyrano[3′,4′:6,7]indolizino[1,2-b]quinoline-3,14(4H)-dione (346 mg, 0.775 mmol, 0.9 eq) and the reaction mixture was stirred at room temperature for 16 h. Progress of the reaction was monitored by TLC. After completion, the reaction mixture was diluted with cold water (50 mL) and the precipitated solid was filtered, dried under vacuum for 5 h and purified by Combiflash column chromatography by eluting with 9% MeOH in DCM to afford (270 mg, 38%) as an off-white solid.

¹H NMR (400 MHz, DMSO-d₆) δ ppm 9.05 (s, 1H) 8.46 (d, J=8.44 Hz, 1H) 8.11 (d, J=8.56 Hz, 1H) 7.77-7.83 (m, 2H) 7.58-7.66 (m, 2H) 7.30-7.36 (m, 2H) 6.94 (d, J=2.32 Hz, 1H) 6.82 (dd, J=9.05, 2.45 Hz, 1H) 6.52 (s, 1H) 5.43 (s, 2H) 5.31 (s, 2H) 4.47 (d, J=13.20 Hz, 2H) 3.97 (d, J=2.45 Hz, 2H) 3.73-3.87 (m, 2H) 3.43 (br s, 4H) 2.96 (t, J=11.98 Hz, 2H) 2.85 (s, 3H) 2.43 (m, 3H) 2.28 (t, J=7.58 Hz, 2H) 1.85-1.95 (m, 4H) 1.72-1.79 (m, 3H) 1.67 (d, J=10.27 Hz, 2H) 1.46-1.52 (m, 2H) 1.17-1.36 (m, 8H) 1.03-1.16 (m, 5H) 0.89 (t, J=7.34 Hz, 3H); LCMS: 99.87%; ESI MS m/z calcd. For C₅₅H₆₃ClN₁₀O₆ ([M+H]⁺) 995.62; found 995.4; HPLC: 357717: 98.86%.

### Example S44. Preparation of N-((1r,4r)-4-(3-chloro-4-cyano-2-methylphenoxy)cyclohexyl)-6-(4-(6-(4-(((S)-4-ethyl-4-hydroxy-3,14-dioxo-3,4,12,14-tetrahydro-1H-pyrano[3′,4′:6,7]indolizino[1,2-b]quinolin-10-yl)methyl)piperazin-1-yl)hexyl)piperidin-1-yl)pyridazine-3-carboxamide (Compound No. 44)

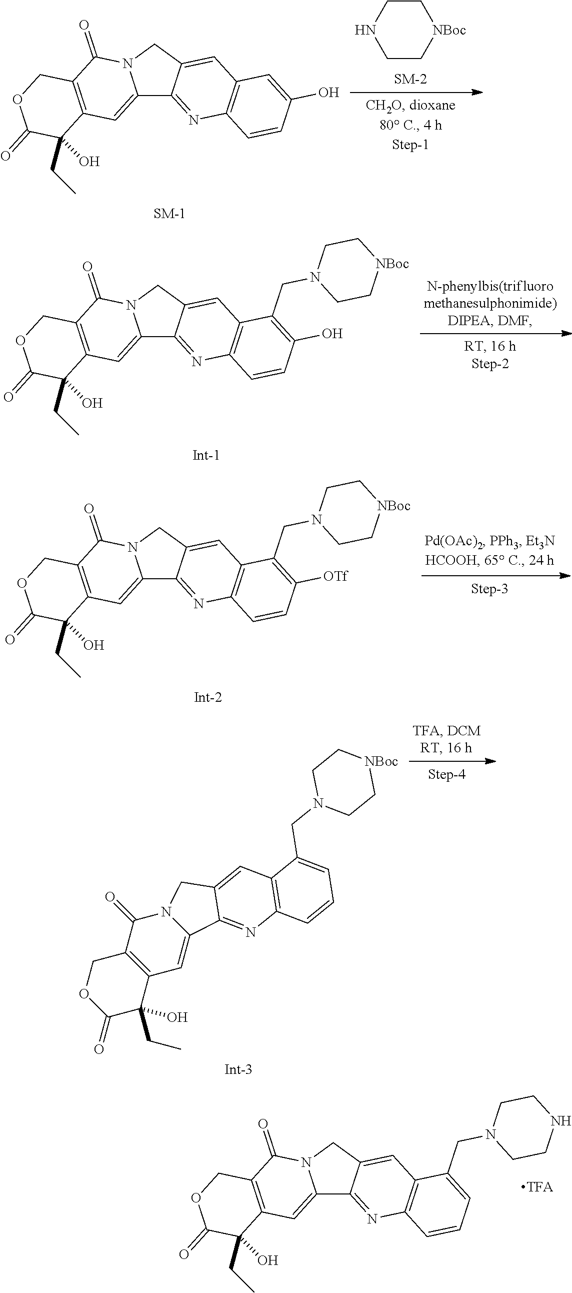 [SMILES: CC[C@@]1(O)C(=O)OCc2c1cc1n(c2=O)Cc2cc3cc(O)ccc3nc2-1 ; CC(C)(C)OC(=O)N1CCNCC1 ; CC[C@@]1(O)C(=O)OCc2c1cc1n(c2=O)Cc2cc3c(CN4CCN(C(=O)OC(C)(C)C)CC4)c(O)ccc3nc2-1 ; CC[C@@]1(O)C(=O)OCc2c1cc1n(c2=O)Cc2cc3c(CN4CCN(C(=O)OC(C)(C)C)CC4)c(OS(=O)(=O)C(F)(F)F)ccc3nc2-1 ; CC[C@@]1(O)C(=O)OCc2c1cc1n(c2=O)Cc2cc3c(CN4CCN(C(=O)OC(C)(C)C)CC4)cccc3nc2-1 ; CC[C@@]1(O)C(=O)OCc2c1cc1n(c2=O)Cc2cc3c(CN4CCNCC4)cccc3nc2-1]

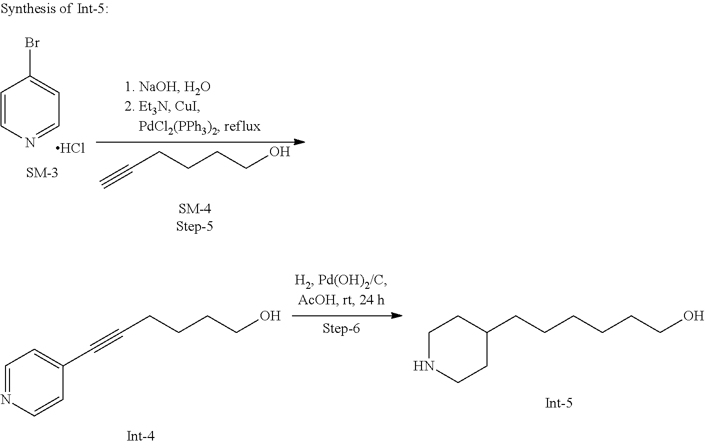

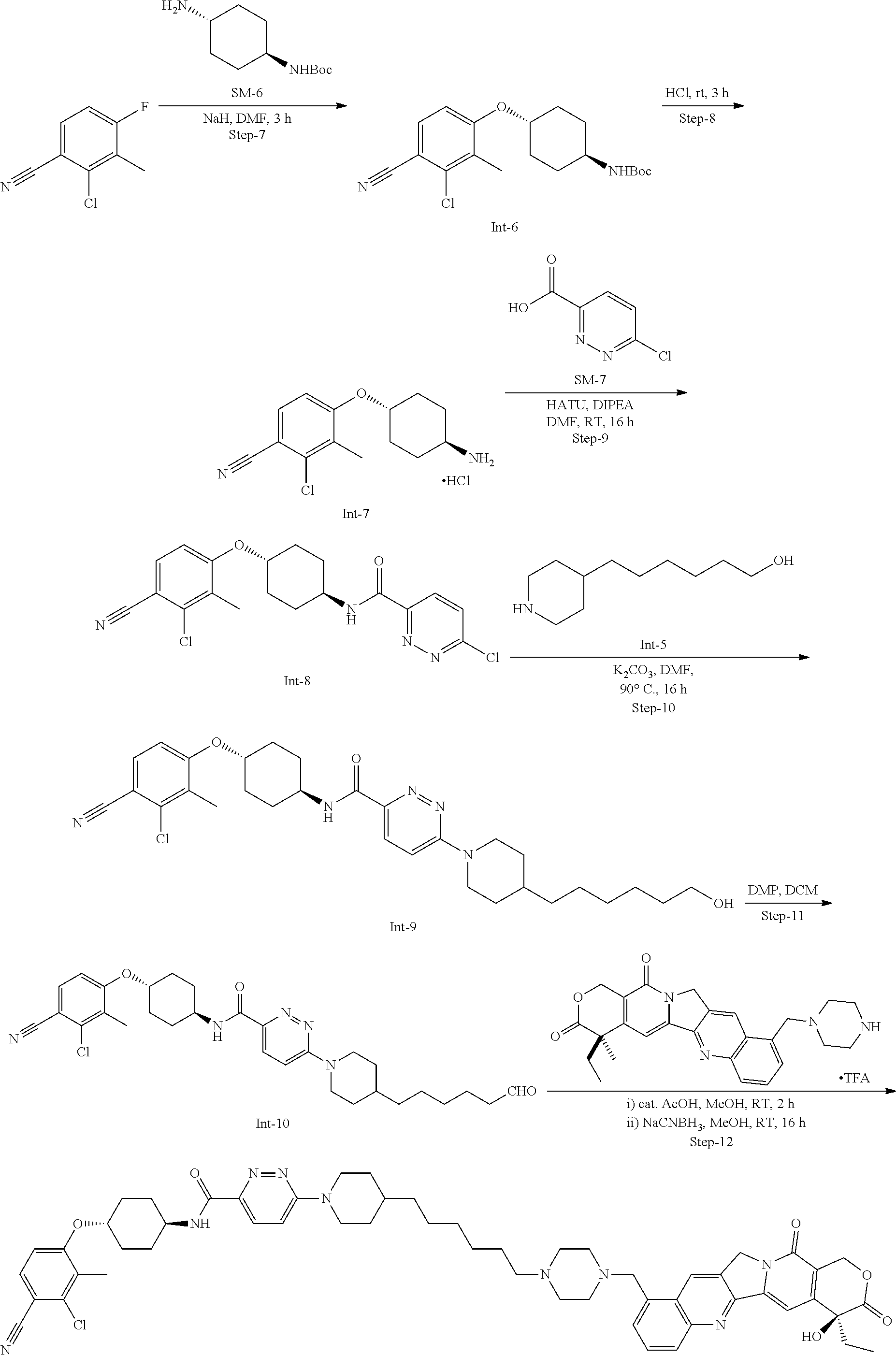

### Step-1: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-1)

A stirred solution of tert-butyl piperazine-1-carboxylate, SM-2 (3.8 g, 20.6 mmol, 1.5 eq) and 5 aqueous formaldehyde solution (37-41%) (1.3 mL, 16.4 mmol, 1.2 eq) in 1,4-dioxane (50 mL) in a sealed tube was heated 80° C. for 1 h. Reaction mixture was cooled to room temperature, (S)-4-ethyl-4,9-dihydroxy-1,12-dihydro-14H-pyrano[3′,4′:6,7]indolizino[1,2-b]quinoline-3,14(4H)-dione, SM-1 (2.0 g, 5.49 mmol, 1 eq) was added portion wise over a period of 5 min and heating at 80° C. was continued for another 4 h. Progress of the reaction was monitored by TLC/LCMS. After completion, the reaction mixture was diluted with water (50 mL) and extracted with DCM (2×75 mL). Combined organic layers were washed with brine (50 mL), water (50 mL), dried over anhydrous Na₂SO₄ and concentrated under reduced pressure to give which was triturated with diethyl ether twice (2×50 mL) to afford Int-1 (4.8 g, 62%) as a yellow colour solid which was used for next step without further purification.

### Step-2: Synthesis of tert-butyl (S)-4-((4-ethyl-4-hydroxy-3,14-dioxo-9-(((trifluoromethyl)sulfonyl)oxy)-3,4,12,14-tetrahydro-1H-pyrano[3′,4′:6,7]indolizino[1,2-b]quinolin-10-yl)methyl)piperazine-1-carboxylate (Int-2)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate, Int-1 (2.0 g, 3.55 mmol, 1 eq), in DMF (20 mL) were added N-phenylbis (trifluoromethanesulphonimide) (1.9 g, 5.33 mmol, 1.5 eq) and DIPEA (1.85 mL, 10.67 mmol, 3 eq) dropwise over a period of 10 min. at 0° C. under nitrogen atmosphere and the reaction mixture was allowed to stir at room temperature for 16 h. Progress of the reaction was monitored by TLC/LCMS. After completion, the reaction mixture was quenched with water (50 mL) and extracted with 10% MeOH in DCM (2×75 mL). Combined organic layers were washed with brine (50 mL), water (50 mL), dried over anhydrous Na₂SO₄ and concentrated under reduced pressure to afford Int-2 (1.48 g, 61%) as an off-white solid.

### Step-3: Synthesis of tert-butyl (S)-4-((4-ethyl-4-hydroxy-3,14-dioxo-3,4,12,14-tetrahydro-1H-pyrano[3′,4′:6,7]indolizino[1,2-b]quinolin-10-yl)methyl)piperazine-1-carboxylate (Int-3)

To a stirred solution of tert-butyl (S)-4-((4-ethyl-4-hydroxy-3,14-dioxo-9-(((trifluoromethyl) sulfonyl)oxy)-3,4,12,14-tetrahydro-1H-pyrano[3′,4′:6,7]indolizino[1,2-b]quinolin-10-yl)methyl)piperazine-1-carboxylate, Int-2 (1.5 g, 2.16 mmol, 1 eq) in DMF (20 mL) were added triphenyl phosphine (56 mg, 0.21 mmol, 0.1 eq). Palladium (II) acetate (24 mg, 0.11 mmol, 0.05 eq) and triethyl amine (0.8 mL, 6.47 mmol, 3 eq) followed by dropwise addition of formic acid (0.34 mL, 8.6 mmol, 4 eq) over a period of 10 min and then the reaction mixture was heated to 65° C. and stirred for 24 h. Progress of the reaction was monitored by TLC/LCMS. After completion, the reaction mixture was quenched with water (50 mL) and extracted with 10% MeOH in DCM (2×75 mL). Combined organic layers were washed with brine (50 mL), water (50 mL), dried over anhydrous Na₂SO₄, filtered, and concentrated under reduced pressure to give crude product which was purified by combiflash column chromatography by eluting with 60% ethyl acetate in heptane to afford Int-3 (630 mg, 53%) as an off-white solid.

### Step-4: Synthesis of (S)-4-ethyl-4-hydroxy-10-(piperazin-1-ylmethyl)-1,12-dihydro-14H-pyrano[3′,4′:6,7]indolizino[1,2-b]quinoline-3,14(4H)-dione. CF₃COOH Salt

A stirred solution of tert-butyl (S)-4-((4-ethyl-4-hydroxy-3,14-dioxo-3,4,12,14-tetrahydro-1H-pyrano[3′,4′:6,7]indolizino[1,2-b]quinolin-10-yl)methyl)piperazine-1-carboxylate, Int-3 (450 mg, 0.824 mmol, 1 eq) in dichloromethane (10 mL) was cooled to 0° C. under inert atmosphere followed by addition of trifluoroacetic acid (0.64 mL, 8.24 mmol, 10 eq) and the reaction mixture was warmed to room temperature and stirred for 16 h. Progress of the reaction was monitored by TLC. After complete consumption of Int-3, solvent was evaporated to obtain crude compound which was triturated with diethyl ether (2×25 mL) to afford TFA salt (280 mg, 76%) as a yellow colour solid.

### Step-5: Synthesis of 6-(pyridin-4-yl)hex-5-yn-1-ol (Int-4)

A solution of 4-bromopyridine. HCl salt, SM-3 (5.0 g, 25.77 mmol, 1 eq) in EtOAc (250 mL) was cooled to 0° C. followed by addition of aqueous sodium hydroxide solution (10.3 g, 257.7 mmol, 10 eq) in water (100 mL) and then the reaction mixture was allowed to stir at room temperature for 30 minutes. After consumption of SM-3 as monitored by TLC, both the layers were separated, and organic layer was washed with brine (100 mL) solution, dried over anhydrous Na₂SO₄, filtered, and concentrated under reduced pressure to give 4-bromopyridine (3.50 g, 86%, crude) (HCl salt free) as colourless liquid.

To a stirred solution of above prepared 4-bromopyridine (3.5 g, 22.29 mmol2, 1 eq) and hex-5-yn-1-ol (2.18 g, 22.29 mmol, 1 eq) in triethyl amine (10 mL) were added copper iodide (84.7 mg, 0.44 mmol, 0.02 eq) and PdCl₂(PPh₃)₂ (156 mg, 0.22 mmol, 0.01 eq) at room temperature and the reaction mixture was heated to 80° C. and stirred for 1 h. Progress of the reaction was monitored by TLC/LCMS. After completion, the reaction mixture was diluted with water (150 mL) and extracted with EtOAc (2×80 mL). The combined organic layers were washed with brine (80 mL), water (75 mL), dried over anhydrous Na₂SO₄, filtered and concentrated under reduced pressure to give crude product which was purified by Combiflash column chromatography by eluting with 70% EtOAc in heptane to afford Int-4 (3.8 g, 97%) as light-yellow oil.

### Step-6: Synthesis of 6-(piperidin-4-yl)hexan-1-ol (Int-5)

To a solution of 6-(pyridin-4-yl)hex-5-yn-1-ol, Int-4 (3.8 g, 21.7 mmol, 1 eq) in acetic acid (80 mL) in a autoclave was added Pd(OH) 2/C (1.60 g) and filled with H₂ gas up to 10 kg and reaction mixture was allowed to stir at ambient temperature for 24 h. Progress of the reaction was monitored by TLC. After completion, the reaction mixture was filtered through a celite bed and washed with EtOAc (100 mL). The combined filtrate was concentrated under reduced pressure to afford Int-5 (3.5 g, 87%) as brown oil which was used as such for next step without any further purification.

### Step-7: Synthesis of tert-butyl ((1r,4r)-4-(3-chloro-4-cyano-2-methyl phenoxy)cyclohexyl) carbamate (Int-6)

To a stirred solution of (trans relative)-tert-butyl ((1r,4r)-4-hydroxycyclohexyl)carbamate, SM-6 (10 g, 46.5 mmol, 1.0 eq) in DMF (100 mL) under nitrogen atmosphere was added NaH (4.08 g, 102 mmol, 2.2 eq) at 0° C. and warmed to room temperature and stirred for 30 min. To this reaction mixture was added 2-chloro-4-fluoro-3-methylbenzonitrile SM-5 (7.86 g, 46.51 mmol, 1.0 eq) portion wise over a period of 10 min at room temperature and stirred for 3 h. Progress of the reaction was monitored by TLC. After completion of starting material, ice cold water (100 mL) was added to this reaction mixture and the precipitated solid formed was filtered and dried to afford Int-6 (15 g, 88%) as an off-white solid.

### Step-8: Synthesis of 4-(((1r,4r)-4-aminocyclohexyl)oxy)-2-chloro-3-methylbenzonitrile hydrochloride (Int-7)

To a solution of tert-butyl ((1r,4r)-4-(3-chloro-4-cyano-2-methylphenoxy)cyclohexyl)carbamate, Int-6 (15 g) was added 4.0M HCl in 1,4-dioxane (75 mL) at 0° C. and stirred for 16 h at room temperature. Progress of the reaction was monitored by TLC. After completion of starting material, volatiles were evaporated under reduced pressure and washed with diethyl ether (2×80 mL) to afford Int-7 (15 g, crude) as an off-white solid which was used for next step without further purification.

### Step-9: Synthesis of 6-chloro-N-((1r,4r)-4-(3-chloro-4-cyano-2-methylphenoxy)cyclohexyl) pyridazine-3-carboxamide (Int-8)

To A mixture of 4-(((1r,4r)-4-aminocyclohexyl)oxy)-2-chloro-3-methylbenzonitrile hydrochloride Int-7 (15 g, 56.9 mmol, 1.0 eq) and 6-chloropyridazine-3-carboxylic acid SM-7 (9 g, 56.9 mmol, 1.0 eq) in DMF (100 mL) were added HATU (32.4 g, 85.44 mmol, 1.5 eq) and DIPEA (49 mL, 284 mmol, 5.0 eq) at 0° C. and the reaction mixture was stirred at room temperature for 16 h. Progress of the reaction was monitored by TLC. After completion of starting material, volatiles were evaporated under reduced pressure, diluted with water (500 mL) and extracted with ethyl acetate (3×300 mL). The combined organic extracts were washed with water (400 mL) and brine (400 mL), dried over anhydrous sodium sulphate, filtered, and concentrated under vacuum to give crude compound which was purified by column chromatography by eluting with 60-80% ethyl acetate in n-heptane to afford Int-8 (6 g, 27%) as an off-white solid.

### Step-10: Synthesis of N-((1r,4r)-4-(3-chloro-4-cyano-2-methylphenoxy)cyclohexyl)-6-(4-(6-hydroxyhexyl)piperidin-1-yl)pyridazine-3-carboxamide (Int-9)

To a stirred solution of 6-chloro-N-((1r,4r)-4-(3-chloro-4-cyano-2-methylphenoxy)cyclohexyl)pyridazine-3-carboxamide Int-8 (250 mg, 0.62 mmol, 1 eq) and 6-(piperidin-4-yl)hexan-1-ol, Int-5 (228 mg, 1.234 mmol, 2 eq) in acetonitrile (5 mL) was added potassium carbonate (603 mg, 1.85 mmol, 3 eq) at room temperature and the reaction mixture was heated to 90° C. and stirred for 16 h. Progress of the reaction was monitored by TLC/LCMS. After completion, the reaction mixture was diluted with water (15 mL) and extracted with EtOAc (2×15 mL). The combined organic extracts were washed with water (15 mL) and brine (15 mL), dried over anhydrous sodium sulphate, filtered, and concentrated under vacuum to afford crude product which was purified by combiflash column chromatography by eluting with 60-65% ethyl acetate in n-heptane to afford Int-9 (200 mg, 58%) as an off-white solid.

### Step-11: Synthesis of N-((1r,4r)-4-(3-chloro-4-cyano-2-methylphenoxy)cyclohexyl)-6-(4-(6-oxohexyl)piperidin-1-yl)pyridazine-3-carboxamide (Int-10)

A stirred solution of N-((1r,4r)-4-(3-chloro-4-cyano-2-methylphenoxy)cyclohexyl)-6-(4-(6-hydroxyhexyl)piperidin-1-yl)pyridazine-3-carboxamide. Int-9 (200 mg, 0.36 mmol, 1 eq) in DCM (10 mL), was cooled to 0° C. and added Dess-Martin periodinane (DMP) (306 mg, 0.723 mmol, 2 eq) portion wise at 0° C. and the reaction mixture was stirred at ambient temperatures for 3 h. Progress of the reaction was monitored by TLC. After completion, the reaction mixture was quenched with 50% aqueous Na₂S₂O₃ solution (10 mL), sat. NaHCO₃ solution (15 mL) and extracted with DCM (2×25 mL). The combined organic extracts were washed with brine (30 mL) and dried over anhydrous sodium sulphate, filtered and concentrated under vacuum to afford Int-10 (180 mg, 90%) as an off-white solid.

### Step-12: Synthesis of N-((1r,4r)-4-(3-chloro-4-cyano-2-methylphenoxy)cyclohexyl)-6-(4-(6-(4-(((S)-4-ethyl-4-hydroxy-3,14-dioxo-3,4,12,14-tetrahydro-1H-pyrano[3′,4′:6,7]indolizino[1,2-b]quinolin-10-yl)methyl)piperazin-1-yl)hexyl)piperidin-1-yl)pyridazine-3-carboxamide

To a stirred solution of N-((1r,4r)-4-(3-chloro-4-cyano-2-methylphenoxy)cyclohexyl)-6-(4-(6-oxohexyl)piperidin-1-yl)pyridazine-3-carboxamide. Int-10 (150 mg, 0.32 mmol, 1.2 eq) in methanol (10 mL) were added(S)-4-ethyl-4-hydroxy-10-(piperazin-1-ylmethyl)-1,12-dihydro-14H-pyrano[3′,4′:6,7]indolizino[1,2-b]quinoline-3,14(4H)-dione (152 mg, 0.27 mmol, 1.0 eq), triethyl amine (0.04 mL, 0.27 mmol, 1 eq) and acetic acid (15 μL. 0.27 mmol, 1 eq) at room temperature and stirred for 3 h. The reaction mixture was then cooled to 0° C. NaCNBH₃ (35 mg, 0.536 mmol, 2 eq) was added portion wise and the reaction mixture was allowed to room temperature and stirred for 16 h. Progress of the reaction was monitored by TLC. After completion, the reaction mixture was diluted with cold water (50 mL), extracted with 10% MeOH in DCM (2×50 mL). The combined organic extracts were washed with water (30 mL), brine (20 mL) and dried over anhydrous sodium sulphate, filtered, and concentrated under vacuum to obtain crude compound which was purified by Combiflash column chromatography by eluting with 15% MeOH in DCM to afford (130 mg, 49%) as an off-white solid.

¹H NMR (400 MHz, DMSO-d₆) δ ppm 9.01 (s, 1H) 8.55 (d, J=8.19 Hz, 1H) 8.11 (d, J=8.44 Hz, 1H) 7.74-7.83 (m, 3H) 7.61-7.67 (m, 1H) 7.30-7.36 (m, 2H) 7.23-7.28 (m, 1H) 6.51-6.53 (m, 1H) 5.43 (s, 2H) 5.30 (s, 2H) 4.43-4.54 (m, 3H) 3.81-4.09 (m, 3H) 3.33-3.51 (m, 2H) 2.91-3.02 (m, 4H) 2.38-2.45 (m, 4H) 2.24 (s, 3H) 2.07-2.15 (m, 3H) 1.85-1.90 (m, 4H) 1.71-1.77 (m, 3H) 1.48-1.70 (m, 6H) 1.18-1.22 (m, 9H) 1.06-1.13 (m, 2H) 0.89 (t, J=7.34 Hz, 3H); LCMS: 95.09%; ESI MS m/z calcd. For C₅₅H₆₄ClN₉O₆ ([M+H]⁺) 982.62; found 982.4; HPLC: 96.04%.

### Example S45. Preparation of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-(((8S,13S,14S,17S)-13-methyl-3-oxo-2,3,6,7,8,11,12,13,14,15,16,17-dodecahydro-1H-cyclopenta[a]phenanthren-17-yl)oxy)hexyl)piperazine-1-carboxylate (Compound No. 45)

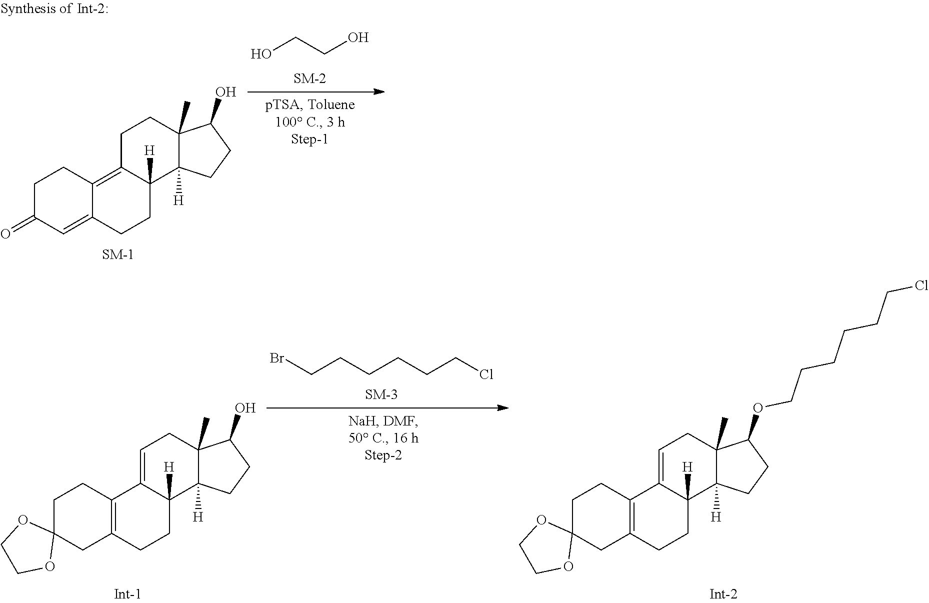

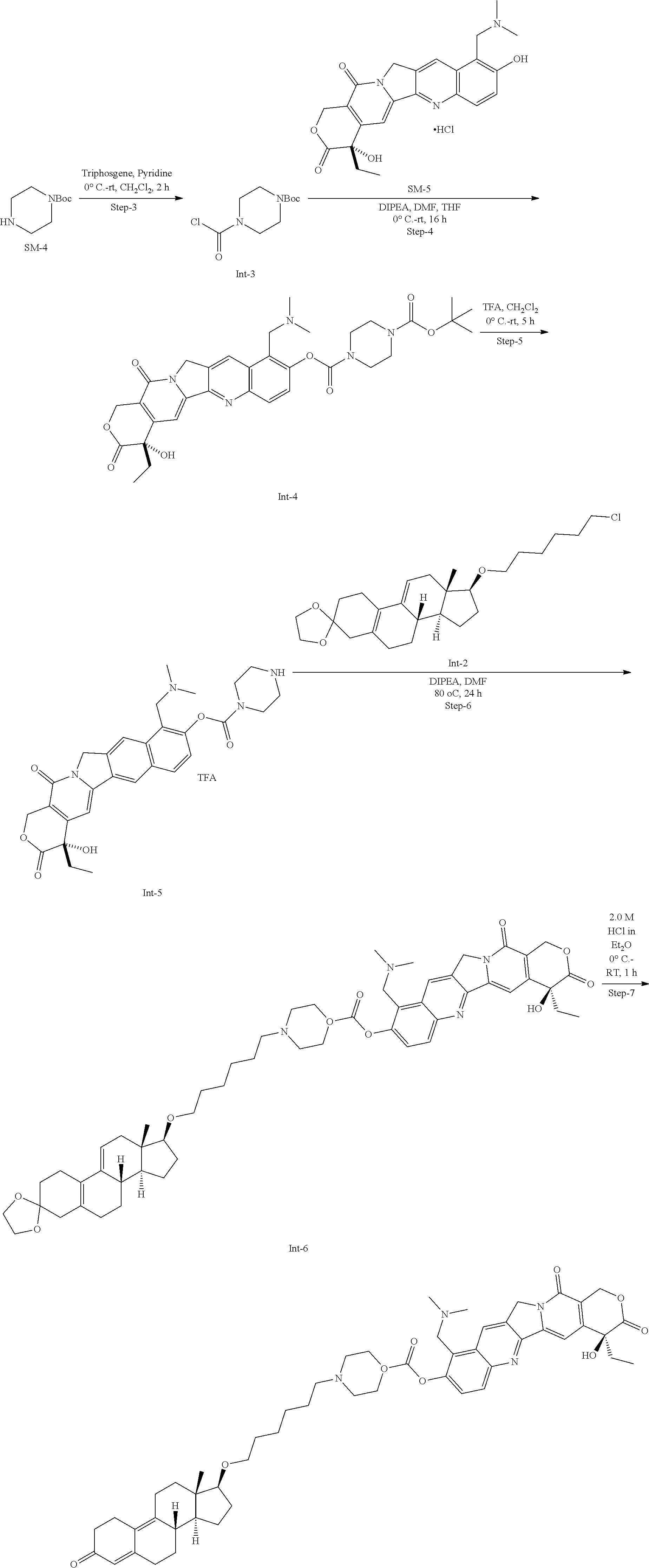

### Step-1: Synthesis of (8S,13S,14S,17S)-13-methyl-1,2,4,6,7,8,12,13,14,15,16,17-dodecahydrospiro[cyclopenta[a]phenanthrene-3,2′-[1,3]dioxolan]-17-ol (Int-1)

To a stirred solution of (8S,13S,14S,17S)-17-hydroxy-13-methyl-1,2,6,7,8,11,12,13,14,15,16,17-dodecahydro-3H-cyclopenta[a]phenanthren-3-one, SM-1 (1.0 g, 3.67 mmol, 1 eq) in toluene (10 mL) were added pTSA (63 mg, 0.36 mmol, 0.1 eq) and ethylene glycol, SM-2 (1.12 mL, 18.35 mmol, 5 eq) and the reaction mixture was allowed to stir at 100° C. for 3 h. Progress of the reaction was monitored by TLC/LCMS. After completion, the reaction mixture was quenched with aqueous sat. NaHCO₃ solution (30 mL) and extracted with EtOAc (2×30 mL). The combined organic layers were washed with water (50 mL), brine (50 mL), dried over anhydrous Na₂SO₄ and concentrated under reduced pressure to give crude compound which was purified by Combiflash column chromatography by eluting with 40% EtOAc in heptane to afford Int-1 (600 mg, 52%) as an off-white solid.

### Step-2: Synthesis of (8S,13S,14S,17S)-17-((6-chlorohexyl)oxy)-13-methyl-1,2,4,6,7,8,12,13,14,15,16,17-dodecahydrospiro[cyclopenta[a]phenanthrene-3,2′-[1,3]dioxolane] (Int-2)

To a stirred solution of (8S,13S,14S,17S)-13-methyl-1,2,4,6,7,8,12,13,14,15,16,17-dodecahydrospiro[cyclopenta[a]phenanthrene-3,2′-[1,3]dioxolan]-17-ol, Int-1 (1.0 g, 3.16 mmol, 1 eq) and 1-bromo-6-chlorohexane. SM-3 (3.15 g, 15.82 mmol, 5 eq) in DMF (10 mL) was added NaH (1.26 g, 31.6 mmol, 10 eq) portion wise at 0° C. After complete addition, the reaction mixture was allowed to stir at 50° C. for 16 h. Progress of the reaction was monitored by TLC/LCMS. After completion, the reaction mixture was cooled to 0° C. quenched with cold water (20 mL) and extracted with ethyl acetate (2×30 mL). The combined organic layers were washed with water (50 mL), brine (50 mL), dried over anhydrous Na₂SO₄ and concentrated under reduced pressure to give crude compound which was purified by Combiflash column chromatography by eluting with 30% EtOAc in heptane to afford Int-2 (640 mg, 46%) as colourless oil.

### Step-3: Synthesis of tert-butyl 4-(chlorocarbonyl)piperazine-1-carboxylate (Int-3)

To a stirred solution of tert-butyl piperazine-1-carboxylate SM-4 (5 g, 26.8 mmol, 1.0 eq) in DCM (100 mL) were added pyridine (2.97 g, 37.6 mmol, 1.4 eq) and triphosgene (3.19 g, 10.7 mmol, 0.4 eq) at 0° C. and allowed to stir at room temperature for 2 h. Progress of the reaction was monitored by TLC. After completion, the reaction mixture was washed with water (100 mL) and extracted with DCM (2×100 mL). The combined organic extracts were again washed with water (200 mL), brine (200 mL) and dried over anhydrous sodium sulphate, filtered, and concentrated under vacuum to afford Int-3 (6.0 g, 90%) as crude oil.

### Step-4: Synthesis of (S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) piperazine-1,4-dicarboxylate (Int-4)

To a stirred solution of (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione. HCl salt SM-5 (10 g, 23.7 mmol, 1.0 eq) in DCM (250 mL) were added DIPEA (15.3 g, 118 mmol, 5 eq) and DMAP (724 mg, 5.9 mmol, 0.25 eq) followed by addition of tert-butyl 4-(chlorocarbonyl)piperazine-1-carboxylate Int-3 (5.89 g, 23.7 mmol, 1 eq) in DCM (25 mL) drop wise over a period of 10 min at 0° C. and stirred for 16 h. Progress of the reaction was monitored by TLC. After completion, the reaction mixture was washed with water (100 mL) and extracted with DCM (3×100 mL). The combined organic extracts were again washed with water (200 mL), brine (200 mL) and dried over anhydrous sodium sulphate, filtered and concentrated under vacuum to provide crude compound which was purified by Combiflash column by using 7% methanol in DCM to afford Int-4 (10 g, 66%) as an off white solid.

### Step-5: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate. TFA salt (Int-5)

To a stirred solution of (S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)piperazine-1,4-dicarboxylate Int-4 (1 g, 15 mmol, 1.0 eq) in DCM (20 mL) under nitrogen atmosphere was added TFA (3 mL) at 0° C. and stirred at room temperature for 16 h. Progress of the reaction was monitored by TLC. After completion, volatiles were evaporated under reduced pressure, sat. aq NaHCO₃ solution (50 mL) was added, extracted with EtOAc (2×100 mL). The combined organic layer was dried over Na₂SO₄, evaporated under reduced pressure to afford Int-5 (1.02 g, crude) as off white solid.

### Step-6: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-(((8S,13S,14S,17S)-13-methyl-1,2,4,6,7,8,12,13,14,15,16,17-dodecahydrospiro[cyclopenta[a]phenanthrene-3,2′-[1,3]dioxolan]-17-yl)oxy)hexyl)piperazine-1-carboxylate (Int-6)

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate Int-5 (250 mg, 0.4685 mmol, 1 eq) and (8S,13S,14S,17S)-17-((6-chlorohexyl)oxy)-13-methyl-1,2,4,6,7,8,12,13,14,15,16,17-dodecahydrospiro[cyclopenta[a]phenanthrene-3,2′-[1,3]dioxolane Int-2 (167 mg., 0.4685 mmol, 1 eq) in DMF (4 mL) at room temperature was added N,N-diisopropylethylamine (0.2 mL, 1.406 mmol, 3 eq) and heated to 80° C. for 24 h. Progress of the reaction was monitored by TLC. TLC showed formation of polar spot as well as presence of starting material. The reaction mixture was quenched with water (20 mL), extracted with dichloromethane (2×30 mL). The combined organic extracts were washed with water (200 mL), brine (200 mL) and dried over anhydrous sodium sulphate, filtered and concentrated under vacuum to give crude compound which was purified by Combiflash column chromatography by eluting with 8-10% methanol in dichloromethane to give Int-6 (80 mg, 14%) as an off-white solid.

### Step-7: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-(((8S,13S,14S,17S)-13-methyl-3-oxo-2,3,6,7,8,11,12,13,14,15,16,17-dodecahydro-1H-cyclopenta[a]phenanthren-17-yl)oxy)hexyl)piperazine-1-carboxylate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-(((8S,13S,14S,17S)-13-methyl-1,2,4,6,7,8,12,13,14,15,16,17-dodecahydrospiro[cyclopenta[a]phenanthrene-3,2′-[1,3]dioxolan]-17-yl)oxy)hexyl)piperazine-1-carboxylate, Int-6 (50 mg, 0.04385 mmol, 1 eq) in DCM (3 mL) at 0° C. was added a solution of 2.0M HCl in diethyl ether (5 mL) and stirred for 2 h at room temperature, Progress of the reaction was monitored by TLC. After completion, the reaction mixture was concentrated, triturated with diethyl ether, and dried to give crude product which was purified by prep. HPLC purification method by eluting with Mobile phase A: 0.1% FA in water and Mobile phase B: Acetonitrile to afford the title compound (16 mg, 34%) as an off-white solid.

¹H NMR (400 MHz, DMSO-d₆) δ 8.94 (s, 1H) 8.10 (d, J=9.13 Hz, 1H) 7.63 (d, J=9.26 Hz, 1H) 7.34 (s, 1H) 6.53 (s, 1H) 5.57 (s, 1H) 5.43 (s, 2H) 5.31 (s, 2H) 3.66-3.80 (m, 4H) 3.47 (s, 2H) 3.39-3.43 (m, 2H) 2.86 (dt, J=14.45, 4.66 Hz, 1H) 2.73-2.82 (m, 1H) 2.44 (d, J=10.63 Hz, 3H) 2.27-2.39 (m, 6H) 2.06-2.26 (m, 9H) 1.79-2.03 (m, 6H) 1.41-1.61 (m, 6H) 1.06-1.37 (m, 9H) 0.89 (t, J=7.25 Hz, 3H) 0.84 (s, 3H); LCMS: 98.17%; ESI MS m/z calcd. For C₅₂H₆₅N₅O₈ ([M+H]⁺) 888.12; found 888.4; HPLC: 99.38%.

### Example S46. Preparation of N-((1r,4r)-4-((3-chloro-4-isocyanophenyl)(methyl)amino)cyclohexyl)-6-(4-(6-(4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazin-1-yl)-6-oxohexyl)piperidin-1-yl)pyridazine-3-carboxamide (Compound No. 46)

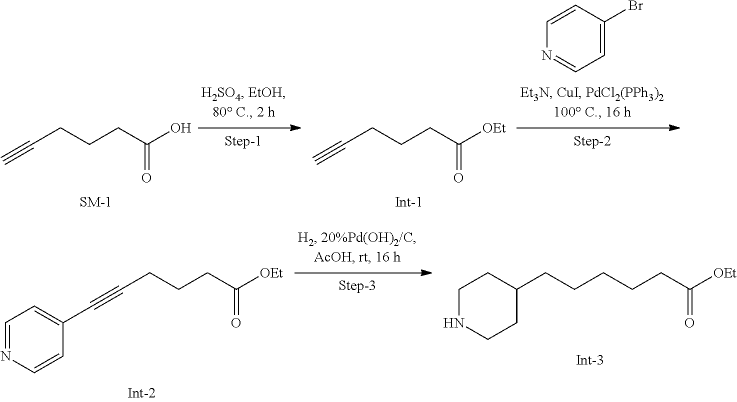

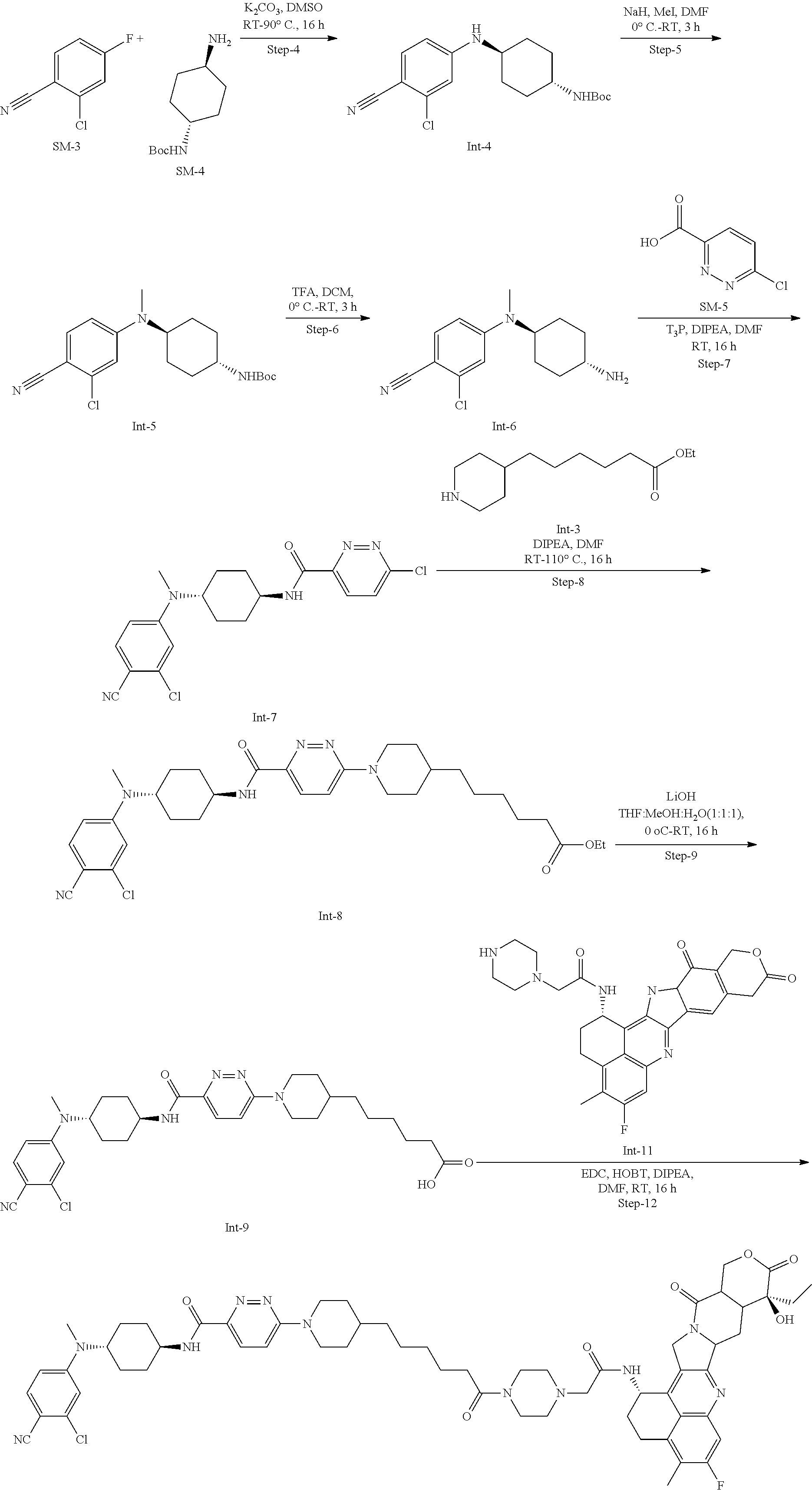

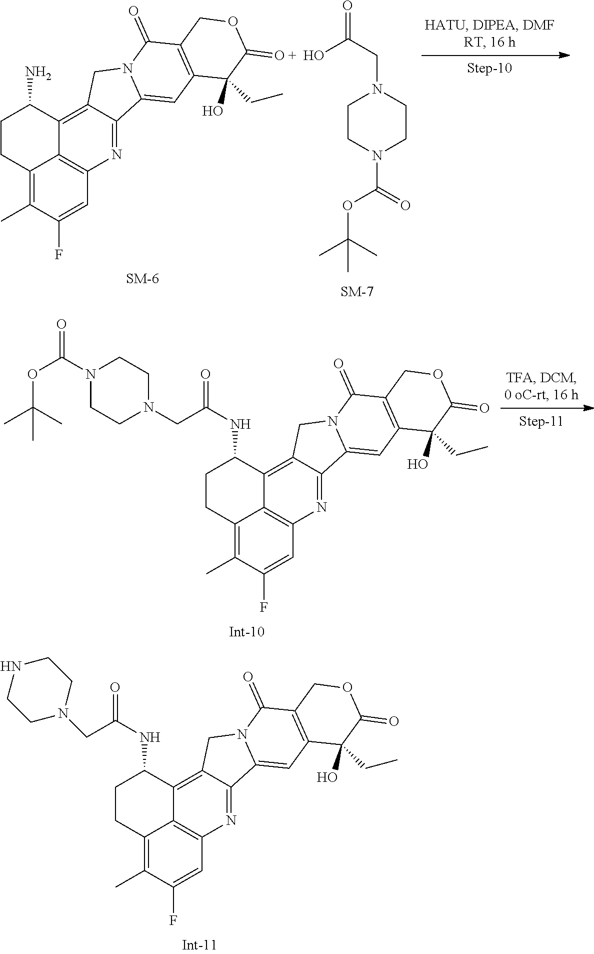

### Step-1: Synthesis of ethyl hex-5-ynoate (Int-1)

To a stirred solution of hex-5-ynoic acid (SM-1, 5 g, 44.5 mmol, 1 eq) in Ethanol (50 mL) was added sulphuric acid (2.38 mL, 44.5 mmol, 1 eq) at 0° C. for 5 min then the resultant reaction mixture was stirred at 80° C. for 2 h. Progress of the reaction was monitored by TLC. After complete consumption of starting material, Ethanol was distilled off under reduced pressure to obtain crude product, which was dissolved in EtOAc (200 mL) and washed with sat. NaHCO₃ solution (100 mL), brine (100 mL), dried over anhydrous Na₂SO₄ and concentrate under reduced pressure to afford ethyl hex-5-ynoate (Int-1, 4 g, 64%) as a pale-yellow liquid.

### Step-2: Synthesis of ethyl 6-(4-pyridyl)hex-5-ynoate (Int-2)

To a stirred solution of ethyl hex-5-ynoate (Int-1, 4 g, 28.5 mmol, 1 eq) in Et₃N (80 mL) was added 4-bromopyridine (SM-2, 4.95 g, 31.3 mmol, 1.1 eq) at room temperature and degassed with Argon for 10 min, after that Copper (I) iodide (54 mg, 0.28 mmol, 0.01 eq) and PdCl₂ (PPh₃)₂ (400 mg, 0.57 mmol, 0.02 eq) was added at room temperature and the resultant reaction mixture was heated to 100° C. and stirred for 16 h. Progress of the reaction was monitored by TLC. After complete consumption of starting materials, the reaction mixture was cooled to room temperature and diluted with EtOAc (200 mL), filtered through celite bed. Filtrate was washed with water (100 mL) and separated the organic layer from aqueous layer, dried over anhydrous Na₂SO₄, filtered, and concentrate under reduced pressure to afford ethyl 6-(4-pyridyl)hex-5-ynoate (Int-2, 5.5 g, 89%) as a yellow liquid.

### Step-3: Synthesis of ethyl 6-(4-piperidyl)hexanoate (Int-3)

To a stirred solution of ethyl 6-(4-pyridyl)hex-5-ynoate (Int-2, 5.5 g, 25.3 mmol, 1.0 eq) in Acetic acid (165 mL) was added 20% Pd(OH)₂ (2.26 g) at room temperature. The resultant reaction mixture was stirred at room temperature for 16 h under Hydrogen atmosphere (100 psi). Progress of the reaction was monitored by TLC. After complete consumption of starting materials, the reaction mixture was filtered through celite bed, cake was washed with 10% MeOH in DCM (100 mL), the filtrate was concentrated under reduced pressure to obtain ethyl 6-(4-piperidyl)hexanoate (Int-3, 3 g, 53%) as a brown colour liquid, which was used in next step, without any further purification.

### Step-4: Synthesis of tert-butyl ((1r,4r)-4-((3-chloro-4-cyanophenyl)amino)cyclohexyl)carbamate (Int-4)

To a stirred solution of 2-chloro-4-fluorobenzonitrile (SM-3, 20 g, 128 mmol, 1.0 eq) in DMSO (200 mL) were added tert-butyl ((1r,4r)-4-aminocyclohexyl)carbamate (SM-4, 27.5 g, 128 mmol, 1.0 eq) and K₂CO₃ (35.3 g, 256 mmol, 2 eq) at room temperature. The resultant reaction mixture was heated to 90° C. for 16 h. Progress of the reaction was monitored by TLC. After complete consumption of the starting materials, the reaction mixture was diluted with ice cold water (1 L) and stirred for 10 min at room temperature. The obtained solids were filtered and washed water, dried under vacuum. The crude solid compound was recrystallized by using EtOH to afford tert-butyl ((1r,4r)-4-((3-chloro-4-cyanophenyl)amino)cyclohexyl)carbamate (Int-4, 30 g, 67%) as an off white solid.

### Step-5: Synthesis of tert-butyl ((1r,4r)-4-((3-chloro-4-cyanophenyl)(methyl)amino)cyclohexyl)carbamate (Int-5)

To a stirred solution of tert-butyl ((1r,4r)-4-((3-chloro-4-cyanophenyl)amino)cyclohexyl) carbamate (Int-4, 30 g, 57.1 mmol, 1.0 eq) in DMF (300 mL) was added 60% NaH (2.96 g, 74.3 mmol, 1.3 eq) under nitrogen atmosphere at 0° C. The resultant reaction mixture was allowed to room temperature and stirred for 30 min. then added methyl iodide (4.6 mL, 74.3 mmol, 1.3 eq) drop wise at 0° C. and allowed to room temperature, stirred for 3 h. Progress of the reaction was monitored by TLC. After complete consumption of starting material. The reaction mixture was diluted with ice cold water (1 L) and stirred for 10 min at room temperature. The obtained solids were filtered and washed with water, dried under vacuum. The obtained crude solid compound was recrystallized by using EtOH to afford tert-butyl ((1r,4r)-4-((3-chloro-4-cyanophenyl)(methyl)amino)cyclohexyl)carbamate (Int-5, 20 g, 64%) as an off white solid.

### Step-6: Synthesis of 4-(((1r,4r)-4-aminocyclohexyl)(methyl)amino)-2-chlorobenzonitrile. TFA Salt (Int-6)

To a stirred solution of tert-butyl ((1r,4r)-4-((3-chloro-4-cyanophenyl)(methyl)amino) cyclohexyl)carbamate (Int-5, 20 g, 54.9 mmol, 1.0 eq) in DCM (300 mL) was added TFA (100 mL, 5 vol) at 0° C., under nitrogen atmosphere. The resultant reaction mixture was allowed to room temperature and stirred for 2 h. Progress of the reaction was monitored by TLC. After complete consumption of starting material, the volatiles were evaporated under reduced pressure to obtain crude compound, which was washed with diethyl ether (20 mL) and dried under vacuum to afford 4-(((1r,4r)-4-aminocyclohexyl)(methyl)amino)-2-chlorobenzonitrile (Int-6, 15 g, crude) as an off white solid.

### Step-7: Synthesis of 6-chloro-N-((1r,4r)-4-((3-chloro-4-cyanophenyl)(methyl)amino)cyclohexyl)pyridazine-3-carboxamide (Int-7)

To a stirred solution of 4-(((1r,4r)-4-aminocyclohexyl)(methyl)amino)-2-chlorobenzonitrile. TFA salt (Int-6, 15 g, 57.03 mmol, 1.0 eq) and 6-chloropyridazine-3-carboxylic acid (SM-5, 9.04 g, 57.03 mmol, 1.0 eq) in DMF (150 mL) were added T3P (36 g, 114 mmol, 2 eq) and DIPEA (30 mL, 171 mmol, 3 eq) at room temperature and stirred for 16 h. Progress of the reaction was monitored by TLC. After complete consumption of the starting materials, the reaction mixture was diluted with ice cold water (1 L) and stirred for 10 min at room temperature, the obtained solids were filtered and washed with water, dried under vacuum to afford 6-chloro-N-((1r,4r)-4-((3-chloro-4-cyanophenyl)(methyl)amino)cyclohexyl)pyridazine-3-carboxamide (Int-7, 13 g, 56%) as an off white solid. ¹H NMR (400 MHz, DMSO-d₆): δ 9.00-9.07 (m, 1H), 8.19-8.25 (m, 1H), 8.07-8.12 (m, 1H), 7.60 (d, J=9.29 Hz, 1H), 6.94 (s, 1H), 6.83 (d, J=8.80 Hz, 1H), 3.66-3.89 (m, 1H), 2.85 (s, 2H), 1.63-1.96 (m, 8H), 1.48-1.63 (m, 1H), 1.11-1.26 (m, 1H), 80.18%; ESI MS m/z calcd. For C₁₉H₁₉Cl₂N₅O ([M+H]⁺) 404.30; found 404.2.

### Step-8: Synthesis of ethyl 6-[1-[6-[[4-(3-chloro-4-cyano-N-methyl-anilino) cyclohexyl]carbamoyl]pyridazin-3-yl]-4-piperidyl]hexanoate (Int-8)

To a stirred solution of 6-chloro-N-((1r,4r)-4-((3-chloro-4-cyanophenyl)(methyl)amino) cyclohexyl)pyridazine-3-carboxamide (Int-7, 3.0 g, 7.42 mmol, 1.0 eq) in DMA (30 mL) were added ethyl 6-(4-piperidyl)hexanoate (Int-3, 2.5 g, 11.1 mmol, 1.5 eq) and DIPEA (1.1 g, 6 mL, 37.1 mmol, 5 eq) at room temperature. The resultant reaction mixture was heated to 110° C. and stirred for 16 h. Progress of the reaction was monitored by TLC. After complete consumption of the starting materials the reaction mixture was diluted with ice cold water (100 mL) and extracted with ethyl acetate (2×150 mL). The combined organic layer was washed with water (200 mL), brine (200 mL) and dried over anhydrous sodium sulphate, filtered, and concentrated under reduced pressure to obtain crude compound, which was purified by combi flash column by eluting with 80% ethyl acetate in henxane to afford 6-[1-[6-[[4-(3-chloro-4-cyano-N-methyl-anilino) cyclohexyl]carbamoyl]pyridazin-3-yl]-4-piperidyl]hexanoate (Int-8, 1.7 g, 38%) as an off white solid.

### Step-9: Synthesis of 6-[1-[6-[[4-(3-chloro-4-cyano-N-methyl-anilino)cyclohexyl]carbamoyl]pyridazin-3-yl]-4-piperidyl]hexanoic Acid (Int-9)

To a stirred solution of ethyl 6-[1-[6-[[4-(3-chloro-4-cyano-N-methyl-anilino) cyclohexyl]carbamoyl]pyridazin-3-yl]-4-piperidyl] hexanoate (Int-8, 1.7 g, 2.85 mmol, 1.0 eq) in THF (20 mL). MeOH (20 mL) and H₂O (20 mL) was added LiOH·H₂O (1.19 g, 28.5 mmol, 10 eq) portion wise at 0° C. The resultant reaction mixture was allowed to room temperature and stirred for 16 h. Progress of the reaction was monitored by TLC. After complete consumption of the starting materials, the volatiles. THF and MeOH were evaporated under reduced pressure to afford crude compound, which was diluted with water (50 mL) and adjusted the PH to 5 by using 1N HCl, the obtained solids were filtered and washed with water. Et₂O, dried under vacuum to afford 6-[1-[6-[[4-(3-chloro-4-cyano-N-methyl-anilino) cyclohexyl] carbamoyl]pyridazin-3-yl]-4-piperidyl] hexanoic acid (Int-9, 1.4 g, 86%) as a white solid.

### Step-10: Synthesis of tert-butyl 4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazine-1-carboxylate (Int-10)

To a stirred solution of (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (SM-6, 500 mg, 1.14 mmol, 1 eq) in DMF (10 mL) were added DIPEA (0.4 mL, 2.29 mmol, 2 eq), HATU (668 mg, 1.72 mmol, 1.5 eq), 2-(4-tert-butoxycarbonylpiperazin-1-yl)acetic acid (SM-7, 308 mg, 1.26 mmol, 1.1 eq) at RT. The resultant reaction mixture was stirred at room temperature for 16 h. Progress of the reaction was monitored by TLC. After complete consumption of starting materials the reaction mixture was poured in ice cold water (50 mL) and the obtained solids were filtered, washed twice with water and dried under vacuum to afford tert-butyl 4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazine-1-carboxylate (Int-10, 525 mg, 84%) as an off white solid.

### Step-11: Synthesis of N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-(piperazin-1-yl)acetamide (Int-11)

To a stirred solution of tert-butyl 4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinoline-1-yl)amino)-2-oxoethyl)piperazine-1-carboxylate (Int-10, 525 mg, 0.79 mmol, 1 eq) in DCM (20 mL) was added TFA (0.12 mL, 2 equiv., 1.58 mmol) at 0° C. The resultant reaction mixture was stirred at room temperature for 16 h. Progress of the reaction was monitored by TLC. After complete consumption of starting material the reaction mixture was concentrated under reduced pressure to obtain N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-(piperazin-1-yl)acetamide (Int-11, 420 mg, 94%) as a green colour solid.

¹H NMR (400 MHz, DMSO-d₆): δ 9.26-9.48 (m, 1H), 8.94-9.12 (m, 1H), 7.82 (d, J=10.76 Hz, 1H), 7.32 (s, 1H), 5.60 (s, 1H), 5.42 (s, 2H), 5.28 (s, 2H), 3.69-4.14 (m, 10H), 3.20 (s, 4H), 2.19 (s, 4H), 1.78-1.91 (m, 2H), 0.85 (d, J=6.36 Hz, 3H), 88.87%; ESI MS m/z calcd. For C₃₀H₃₂FN₅O₅ ([M+H]+) 561.61; found 562.5.

### Step-12: N-((1r,4r)-4-((3-chloro-4-isocyanophenyl)(methyl)amino)cyclohexyl)-6-(4-(6-(4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazin-1-yl)-6-oxohexyl)piperidin-1-yl)pyridazine-3-carboxamide

To a stirred solution of N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-(piperazin-1-yl)acetamide (Int-11, 200 mg, 0.35 mmol, 1 eq) in DMF (15 mL) were added DIPEA (0.31 mL, 1.781 mmol, 5 eq). HOBT (57 mg, 0.42 mmol, 1.2 eq), EDC. HCl (75 mg, 0.39 mmol, 1.1 eq) and 6-[1-[6-[[4-(3-chloro-4-cyano-N-methyl-anilino) cyclohexyl] carbamoyl]pyridazin-3-yl]-4-piperidyl] hexanoic acid (Int-9, 140 mg, 0.24 mmol, 0.7 eq) at room temperature. The resultant reaction mixture was stirred at room temperature for 16 h. Progress of the reaction was monitored by TLC. After complete consumption of starting materials, the reaction mixture was poured into ice cold water (50 ml) and extracted with 10% MeOH in DCM. The combined organic layers were washed with water (50 ml), brine (50 mL) and dried over anhydrous Na₂SO₄, filtered and concentrate under reduced pressure to afford crude compound, which was purified by prep. HPLC purification by eluting with Mobile phase A: 0.1% NH₄HCO₃ in water and Mobile phase B: Acetonitrile to afford the title compound (123.9 mg, 31.32%) as an off white solid.

¹H NMR (400 MHz, DMSO-d₆): δ 8.49 (dd, J=19.76, 8.63 Hz, 2H), 7.76-7.82 (m, 2H), 7.60 (d, J=9.01 Hz, 1H), 7.29-7.33 (m, 2H), 6.94 (d, J=2.38 Hz, 1H), 6.82 (dd, J=9.07, 2.44 Hz, 1H), 6.52 (s, 1H), 5.54-5.60 (m, 1H), 5.42 (s, 2H), 5.15-5.27 (m, 2H), 4.46 (d, J=13.51 Hz, 2H), 3.72-3.90 (m, 2H), 3.40-3.50 (m, 4H), 3.13-3.21 (m, 2H), 3.04-3.13 (m, 2H), 2.95 (t, J=11.82 Hz, 2H), 2.85 (s, 3H), 2.53-2.55 (m, 1H), 2.46 (s, 2H), 2.39 (s, 3H), 2.28 (t, J=7.44 Hz, 2H), 2.16 (q, J=6.09 Hz, 2H), 1.82-1.94 (m, 4H), 1.61-1.78 (m, 8H), 1.43-1.61 (m, 4H), 1.16-1.34 (m, 6H), 1.03-1.16 (m, 2H), 0.87 (t, J=7.32 Hz, 3H), 95.10%; ESI MS m/z calcd. For C60H69ClFN11O7 ([M+H]+) 1110.73; found 1111.70; HPLC: 96.76%.

### Example S47. Preparation of (8S,11R,13S,14S,17R)-17-acetyl-11-(4-((6-(2-(((1S,9S)-9-ethyl-5-fluoro-9-hydroxy-4-methyl-10,13-dioxo-2,3,9,10,13,15-hexahydro-1H,12H-benzo[de]pyrano [3′,4′:6,7]indolizino[1,2-b]quinolin-1-yl)amino)-2-oxoethoxy)hexyl)(methyl)amino)phenyl)-13-methyl-3-oxo-2,3,6,7,8,11,12,13,14,15,16,17-dodecahydro-1H-cyclopenta[a]phenanthren-17-yl acetate (Compound No. 47)

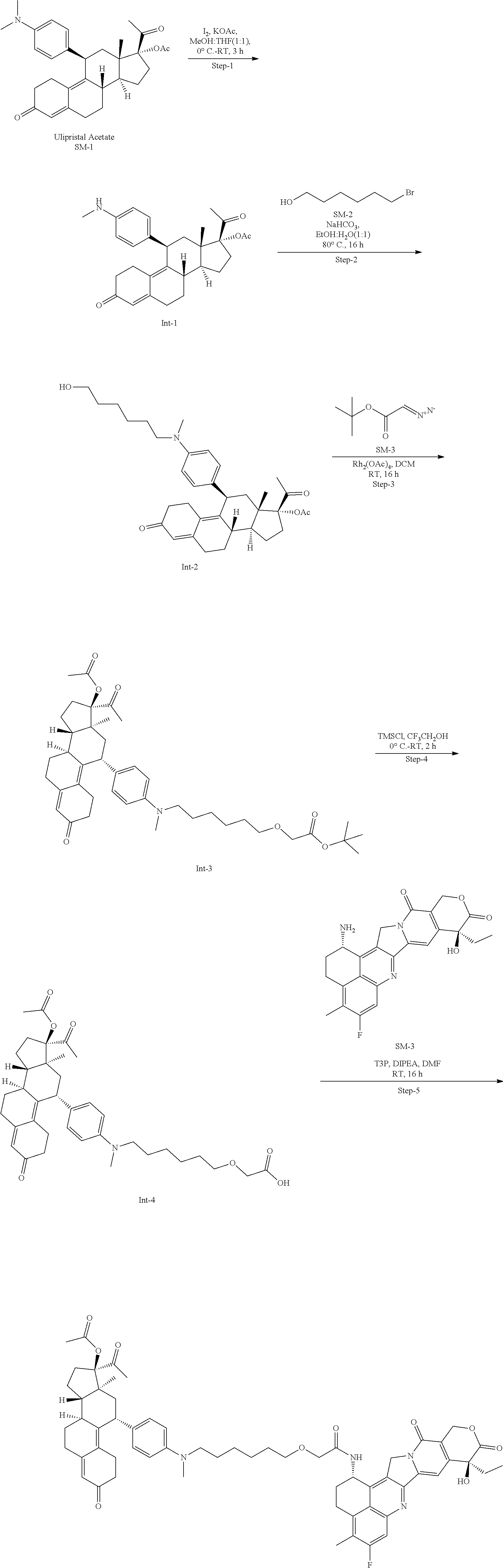

### Step-1: Synthesis of (8S,11R,13S,14S,17R)-17-acetyl-13-methyl-11-(4-(methylamino)phenyl)-3-acetate oxo-2,3,6,7,8,11,12,13,14,15,16,17-dodecahydro-1H-cyclopenta[a]phenanthren-17-yl (Int-1)

To a stirred solution of Ulipristal acetate (SM-1, 10 g, 21 mmol) in methanol (150 mL) and THF (150 mL) were added KOAc (20.6 g, 210 mmol) and Iodine (13.1 g, 105 mmol, 5 eq) at 0° C. The resultant reaction mixture was allowed to room temperature and stirred for 3 h. Progress of the reaction was monitored by TLC. After complete consumption of the starting material, the reaction mixture was quenched with sodium thiosulfate (Na₂S₂O₃) solution (50 g in 200 mL water) and extracted with ethyl acetate (2×200 mL). The combined organic layer was washed with brine (200 mL) and dried over anhydrous sodium sulphate, filtered and concentrated under reduced pressure to (8S,11R,13S,14S,17R)-17-acetyl-13-methyl-11-(4-(methylamino)phenyl)-3-oxo-afford 2,3,6,7,8,11,12,13,14,15,16,17-dodecahydro-1H-cyclopenta[a]phenanthren-17-yl acetate (Int-1, 8.0 g, 82%) as an off-white solid which was used in next step without any further purification.

### Step-2: Synthesis of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2)

To a solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 4 g, 8.67 mmol) and 6-bromohexan-1-ol (SM-2, 7.81 g, 43.38 mmol) in ethanol (40 mL) and water (40 mL) was added NaHCO₃ (7.37 g, 86.76 mmol) at room temperature. The resultant reaction mixture was heated to 80° C. and stirred for 16 h. Progress of the reaction was monitored by TLC. After complete consumption of the starting materials, the reaction mixture was filtered through a celite bed and washed with ethyl acetate (100 mL). Filtrate was concentrated under reduced pressure and diluted with water (200 mL), extracted with ethyl acetate (2×200 mL). The combined organic layer was washed with brine (100 mL) and dried over anhydrous sodium sulphate, filtered and concentrated under reduced pressure to obtain crude compound, which was purified by combiflash chromatography by eluting with 70% ethyl acetate in heptane to afford (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 2.6 g, 53%) as an off-white solid.

### Step-3: Synthesis of tert-butyl 2-((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)oxy)acetate (Int-3)

To a stirred solution of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 1.0 g, 1.8 mmol) in DCM (10 mL). Rhodium (II) Acetate Dimer (16 mg, 0.36 mmol) and tert-butyl 2-diazoacetate (SM-3, 0.63 g, 4.4 mmol) was added at ambient temperature under argon atmosphere. The resulting reaction mixture stirred at RT for 16 h. Progress of the reaction was monitored by TLC. After complete consumption of the starting materials, the reaction mixture was diluted with ice cold water (200 mL), extracted with DCM (2×100 mL). The combined organic layer was washed with brine solution (100 mL), dried over anhydrous sodium sulphate, filtered and concentrated under reduced pressure to obtain crude compound, which was purified by column chromatography to obtain tert-butyl 2-((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl) (methyl)amino)hexyl)oxy)acetate (Int-3, 500 mg, 0.73 mmol, 42%) as a brown liquid.

### Step-4: Synthesis of 2-((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)oxy)acetic acid (Int-4)

To a stirred solution of tert-butyl 2-((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)oxy)acetate (Int-3, 500 mg, 0.73 mmol) in 2,2,2-Trifluoroethanol (5 mL) was added Chlorotrimethylsilane (1.99 ml, 14.80 mmol) at 0° C. under argon atmosphere. Then the reaction mixture was allowed to room temperature and stirred for 2 h. Progress of the reaction was monitored by TLC. After complete consumption of the starting materials. The reaction mixture was directly distilled and triturated with n-pentane and dried to obtain 2-((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)oxy)acetic acid (Int-4) (380 mg, 0.61 mmol, 82.88%) as a brown solid.

### Step-5: Synthesis of (8S,11R,13S,14S,17R)-17-acetyl-11-(4-((6-(2-(((1S,9S)-9-ethyl-5-fluoro-9-hydroxy-4-methyl-10,13-dioxo-2,3,9,10,13,15-hexahydro-1H,12H-benzo[de]pyrano [3′,4′:6,7]indolizino[1,2-b]quinolin-1-yl)amino)-2-oxoethoxy)hexyl)(methyl)amino)phenyl)-13-methyl-3-oxo-2,3,6,7,8,11,12,13,14,15,16,17-dodecahydro-1H-cyclopenta[a]phenanthren-17-yl acetate

To a stirred solution of 2-((6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl) (methyl)amino)hexyl)oxy)acetic acid (Int-4, 350 mg, 0.5647 mmol) in DMF (7 mL), were added DIPEA (0.31 ml, 1.694 mmol) and 2,4,6-tripropyl-1,3,5,2,4,6-trioxatriphosphorinane-2,4,6-trioxide in ethyl acetate (537 mg, 1.694 mmol) and stirred for 10 minutes at room temperature. Then (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (SM-3, 243 mg, 0.56 mmol) was added at room temperature. The resultant reaction mixture was stirred at ambient temperature for 16 h. Progress of the reaction was monitored by TLC. After complete consumption of starting materials, the reaction mixture was quenched with ice-cold water, extracted with EtOAc (2×50 mL). The combined organic layer was washed with brine solution (50 mL), dried over anhydrous Na₂SO₄, filtered and concentrated under reduced pressure to obtain crude product which was purified by (8S,11R,13S,14S,17R)-17-acetyl-11-(4-((6-(2-reverse phase column purification to obtain (((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)hexyl) (methyl)amino)phenyl)-13-methyl-3-oxo-2,3,6,7,8,11,12,13,14,15,16,17-dodecahydro-1H-cyclopenta[a]phenanthren-17-yl acetate (75 mg, 0.072 mmol, 12.81%) as a white solid.

¹H NMR (400 MHz, DMSO-d₆): δ 8.42-8.51 (m, 2H), 7.79 (d, J=11.01 Hz, 1H), 7.27-7.31 (m, 1H), 6.92 (d, J=8.63 Hz, 2H), 6.48-6.55 (m, 3H), 5.66 (s, 1H), 5.55-5.62 (m, 1H), 5.38 (d, J=7.00 Hz, 2H), 5.20 (d, J=4.13 Hz, 2H), 4.37 (d, J=6.63 Hz, 1H), 3.97 (s, 2H), 3.44-3.50 (m, 2H), 3.13-3.20 (m, 4H), 2.68-2.84 (m, 5H), 2.34-2.41 (m, 5H), 2.09-2.24 (m, 8H), 2.07 (s, 1H), 2.02-2.07 (m, 1H), 1.93-2.01 (m, 5H), 1.85-1.92 (m, 1H), 1.80 (dt, J=10.69, 6.72 Hz, 2H), 1.61-1.73 (m, 2H), 1.46-1.54 (m, 2H), 1.35-1.44 (m, 3H), 1.19-1.34 (m, 7H), 0.84 (t, J=7.32 Hz, 3H), LCMS: 97.18%; ESI MS m/z calcd. For C₆₁H₆₉FN₄O₁₀ ([M+H]⁺) 1037.24; found 1037.7; HPLC: 95.08%.

### Example S48. Preparation of N-((1r,4r)-4-(3-chloro-4-cyano-2-methylphenoxy)cyclohexyl)-6-(4-(6-(4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazin-1-yl)hexyl)piperidin-1-yl)pyridazine-3-carboxamide (Compound No. 48)

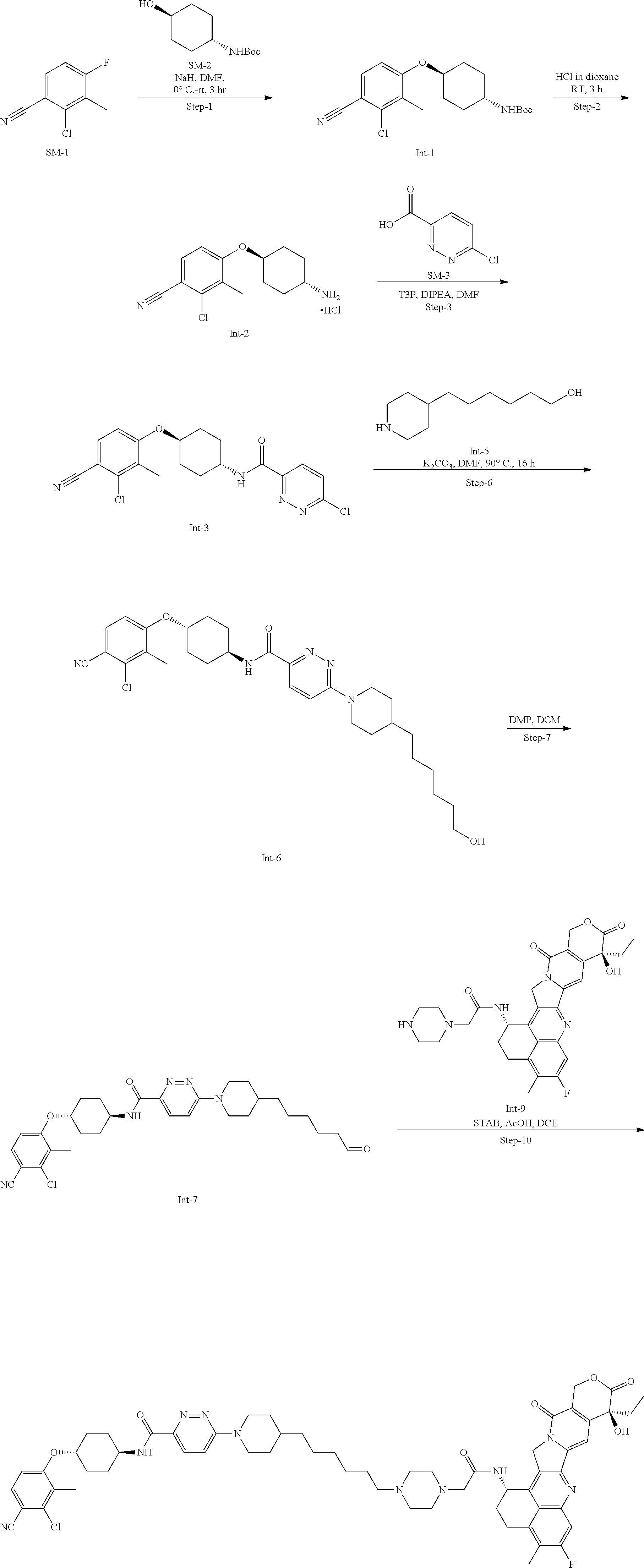

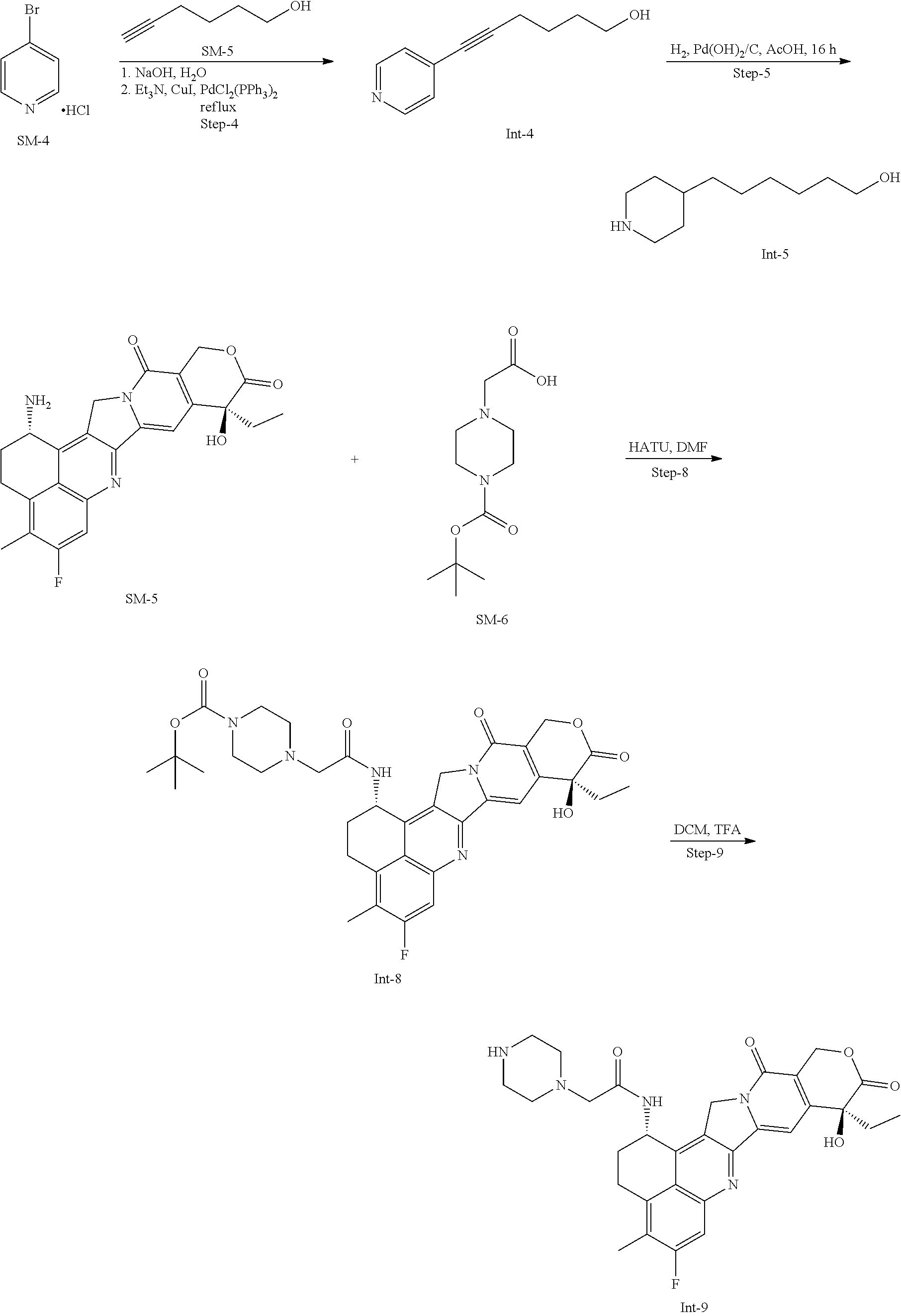

### Step-1: Synthesis of tert-butyl ((1r,4r)-4-(3-chloro-4-cyano-2-methyl phenoxy)cyclohexyl) carbamate (Int-1)

To a stirred solution of tert-butyl ((1r,4r)-4-hydroxycyclohexyl)carbamate (SM-2, 10 g, 46.5 mmol) in DMF (100 mL) under nitrogen atmosphere was added NaH (60% in mineral oil, 4.08 g, 102 mmol) at 0° C. and warmed to room temperature and stirred for 30 min. And then 2-chloro-4-fluoro-3-methylbenzonitrile (SM-1, 7.86 g, 46.51 mmol) was added portion wise over a period of 10 min at room temperature and stirred for 3 h. Progress of the reaction was monitored by TLC. After complete consumption of starting material, the reaction mixture was poured in to ice cold water (300 mL) and the obtained solids were filtered and dried under vacuum to afford tert-butyl ((1r,4r)-4-(3-chloro-4-cyano-2-methyl phenoxy)cyclohexyl)carbamate (Int-1, 15 g, 88%) as an off-white solid.

### Step-2: Synthesis of 4-(((1r,4r)-4-aminocyclohexyl)oxy)-2-chloro-3-methylbenzonitrile hydrochloride (Int-2)

To a stirred solution of tert-butyl ((1r,4r)-4-(3-chloro-4-cyano-2-methylphenoxy)cyclohexyl)carbamate (Int-1, 15 g, 41.2 mmol) was added 4M HCl in 1,4-dioxane (75 mL) at 0° C. and the resultant reaction mixture was stirred at RT for 16 h. Progress of the reaction was monitored by TLC. After complete consumption of starting material, the volatiles were evaporated under reduced pressure to obtain crude compound, which was washed with diethyl ether (2×80 mL) to afford 4-(((1r,4r)-4-aminocyclohexyl)oxy)-2-chloro-3-methylbenzonitrile hydrochloride (Int-2, 15 g, crude) as an off-white solid, which was used for next step without any further purification.

### Step-3: Synthesis of 6-chloro-N-((1r,4r)-4-(3-chloro-4-cyano-2-methylphenoxy)cyclohexyl) pyridazine-3-carboxamide (Int-3)

To the mixture of 4-(((1r,4r)-4-aminocyclohexyl)oxy)-2-chloro-3-methylbenzonitrile hydrochloride (Int-2, 15 g, 56.9 mmol) and 6-chloropyridazine-3-carboxylic acid (SM-3, 9 g, 56.9 mmol) in DMF (100 mL) were added HATU (32.4 g, 85.44 mmol) and DIPEA (49 mL, 284 mmol) at 0° C. The resultant reaction mixture was at room temperature for 16 h. Progress of the reaction was monitored by TLC. After complete consumption of starting material, the volatiles were evaporated under reduced pressure and diluted with water (500 mL) and extracted with ethyl acetate (3×300 mL). The combined organic extracts were washed with water (400 mL), brine (400 mL), dried over anhydrous sodium sulphate, filtered, and concentrated under reduced pressure to obtain crude compound, which was purified by column chromatography by eluting with 60-80% ethyl acetate in n-heptane to afford 6-chloro-N-((1r,4r)-4-(3-chloro-4-cyano-2-methylphenoxy)cyclohexyl)pyridazine-3-carboxamide (Int-3, 6 g, 27%) as an off-white solid.

### Step-4: Synthesis of 6-(pyridin-4-yl)hex-5-yn-1-ol (Int-4)

A solution of 4-bromopyridine. HCl salt (SM-3, 5.0 g, 25.77 mmol) in EtOAc (250 mL) was cooled to 0° C. followed by addition of aqueous sodium hydroxide solution (10.3 g, 257.7 mmol) in water (100 mL) and then the resulted reaction mixture was allowed to stir at room temperature for 30 minutes. After consumption of SM-3 as monitored by TLC, both the layers were separated and organic layer was washed with brine (100 mL) solution, dried over anhydrous Na₂SO₄, filtered, and concentrated under reduced pressure to give 4-bromopyridine (3.50 g, 86%, crude) (HCl salt free) as colourless liquid.

To a stirred solution of above prepared 4-bromopyridine (3.5 g, 22.29 mmol) and hex-5-yn-1-ol (2.18 g, 22.29 mmol) in triethyl amine (10 mL) were added copper iodide (84.7 mg, 0.44 mmol) and PdCl₂(PPh₃)₂ (156 mg, 0.22 mmol) at room temperature and the reaction mixture was heated to 80° C. and stirred for 1 h. Progress of the reaction was monitored by TLC/LCMS. After complete consumption of starting materials, the reaction mixture was diluted with water (150 mL) and extracted with EtOAc (2×80 mL). The combined organic layer was washed with water (75 mL), brine (80 mL) and dried over anhydrous Na₂SO₄, filtered and concentrated under reduced pressure to give crude product, which was purified by combi-flash column chromatography by eluting with 70% EtOAc in heptane to afford 6-(pyridin-4-yl)hex-5-yn-1-ol (Int-4, 3.8 g, 97%) as a light-yellow oil.

### Step-5: Synthesis of 6-(piperidin-4-yl)hexan-1-ol (Int-5)

To a solution of 6-(pyridin-4-yl)hex-5-yn-1-ol (Int-4, 3.8 g, 21.7 mmol) in acetic acid (80 mL) in a autoclave was added Pd(OH) 2/C (1.60 g) and filled with H₂ gas (100 PSI) and the resultant reaction mixture was allowed to stir at ambient temperature for 24 h. Progress of the reaction was monitored by TLC. After complete consumption of starting material, the reaction mixture was filtered through a celite bed and washed with EtOAc (100 mL). The combined filtrate was concentrated under reduced pressure to afford 6-(piperidin-4-yl)hexan-1-ol (Int-5, 3.5 g, 87%) as a brown oil, which was used as such for next step without any further purification.

### Step-6: Synthesis of N-((1r,4r)-4-(3-chloro-4-cyano-2-methylphenoxy)cyclohexyl)-6-(4-(6-hydroxyhexyl)piperidin-1-yl)pyridazine-3-carboxamide (Int-6)

To a stirred solution of 6-chloro-N-((1r,4r)-4-(3-chloro-4-cyano-2-methylphenoxy)cyclohexyl)pyridazine-3-carboxamide (Int-3, 250 mg, 0.62 mmol) and 6-(piperidin-4-yl)hexan-1-ol (Int-5, 228 mg, 1.234 mmol) in acetonitrile (5 mL) was added potassium carbonate (603 mg, 1.85 mmol) at room temperature and the resultant reaction mixture was heated to 90° C. and stirred for 16 h. Progress of the reaction was monitored by TLC/LCMS. After complete consumption of starting materials, the reaction mixture was diluted with water (50 mL) and extracted with EtOAc (2×30 mL). The combined organic extracts were washed with water (30 mL) and brine (15 mL), dried over anhydrous sodium sulphate, filtered, and concentrated under reduced pressure to afford crude product, which was purified by combi-flash column chromatography by eluting with 60-65% ethyl acetate in n-heptane to afford N-((1r,4r)-4-(3-chloro-4-cyano-2-methylphenoxy)cyclohexyl)-6-(4-(6-hydroxyhexyl)piperidin-1-yl)pyridazine-3-carboxamide (Int-6, 200 mg, 58%) as an off-white solid.

### Step-7: Synthesis of N-((1r,4r)-4-(3-chloro-4-cyano-2-methylphenoxy)cyclohexyl)-6-(4-(6-oxohexyl)piperidin-1-yl)pyridazine-3-carboxamide (Int-7)

To a stirred solution of N-((1r,4r)-4-(3-chloro-4-cyano-2-methylphenoxy)cyclohexyl)-6-(4-(6-hydroxyhexyl)piperidin-1-yl)pyridazine-3-carboxamide (Int-6, 200 mg, 0.36 mmol) in DCM (10 mL) was added Dess-Martin periodinane (DMP, 306 mg, 0.723 mmol) portion wise at 0° C. and the resultant reaction mixture was stirred at ambient temperatures for 3 h. Progress of the reaction was monitored by TLC. After complete consumption of starting material, the reaction mixture was quenched with 50% aqueous Na₂S₂O₃ solution (10 mL), sat. NaHCO₃ solution (30 mL) and extracted with DCM (2×30 mL). The combined organic extracts were washed with brine (30 mL) and dried over anhydrous sodium sulphate, filtered and concentrated under reduced pressure to afford N-((1r,4r)-4-(3-chloro-4-cyano-2-methylphenoxy)cyclohexyl)-6-(4-(6-oxohexyl)piperidin-1-yl)pyridazine-3-carboxamide (Int-7, 180 mg, 90%) as an off-white solid.

### Step-8: Synthesis of tert-butyl 4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazine-1-carboxylate (Int-8)

To a stirred solution of (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (SM-5, 500 mg, 1.14 mmol) in DMF (10 mL) were added DIPEA (0.4 mL, 2.29 mmol). HATU (668 mg, 1.72 mmol) and 2-(4-{tert}-butoxycarbonylpiperazin-1-yl)acetic acid (SM-6, 308 mg, 1.26 mmol) at room temperature. The resultant reaction mixture was stirred at room temperature for 16 h. Progress of the reaction was monitored by TLC. After complete consumption of starting materials, the reaction mixture poured in to ice cold water (100 mL) and the precipitated solid was filtered and washed twice with water, dried under vacuum to afford tert-butyl 4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazine-1-carboxylate (Int-8, 525 mg, 84%) as an off white solid.

### Step-9: Synthesis of N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-(piperazin-1-yl)acetamide (Int-9)

To a stirred solution of tert-butyl 4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazine-1-carboxylate (Int-8, 525 mg, 0.79 mmol) in DCM (20 mL) was added TFA (0.12 mL, 1.58 mmol) at 0° C. The resultant reaction mixture was stirred at room temperature for 16 h. Progress of the reaction was monitored by TLC. After complete consumption of starting material, the volatiles were evaporated under reduced pressure and the obtained solid was washed with diethyl ether (2×80 mL) to afford N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-(piperazin-1-yl)acetamide (Int-9, 420 mg, 94%) as a green colour solid.

### Step-10: Synthesis of N-((1r,4r)-4-(3-chloro-4-cyano-2-methylphenoxy)cyclohexyl)-6-(4-(6-(4-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)piperazin-1-yl)hexyl)piperidin-1-yl)pyridazine-3-carboxamide

To a stirred solution of N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-(piperazin-1-yl)acetamide (Int-9, 244 mg, 0.43 mmol) in DCE (10 mL) was added Et₃N (0.25 mL, 1.81 mmol) at 0° C. and warmed it to room temperature, stirred for 2 h. After that N-((1r,4r)-4-(3-chloro-4-cyano-2-methylphenoxy)cyclohexyl)-6-(4-(6-oxohexyl)piperidin-1-yl)pyridazine-3-carboxamide (Int-7, 200 mg, 0.36 mmol) and acetic acid (0.02 mL, 0.36 mmol) were added at 0° C. The resultant reaction mixture was stirred at room temperature for 2 h. After that sodium triacetoxy borohydride (191 mg, 0.90 mmol) was added at 0° C. The resultant reaction mixture was stirred at room temperature for 16 h. Progress of the reaction was monitored by TLC. After complete consumption of starting materials, the reaction mixture was cooled to 0° C. and quenched with sat. NaHCO₃ solution (10 mL), extracted with 10% MeOH in DCM (2×50 mL). The combined organic layer was washed with water (20 mL) and brine (20 mL), dried over anhydrous Na₂SO₄ and concentrated under reduced pressure to obtain crude product as a pale brown solid, which was purified by reverse phase purification (using 0.1% FA in water eluted in 55% Acetonitrile in 0.1% FA in water) to afford the title compound (70 mg, 17.6%) as an off white solid.

¹H NMR (400 MHz, DMSO-d₆): δ 8.56 (d, J=8.19 Hz, 1H), 8.39-8.48 (m, 1H), 7.74-7.82 (m, 3H), 7.23-7.34 (m, 3H), 6.52 (d, J=2.93 Hz, 1H), 5.52-5.60 (m, 1H), 5.42 (s, 2H), 5.20 (s, 2H), 4.42-4.55 (m, 3H), 3.81-3.93 (m, 1H), 2.91-3.19 (m, 6H), 2.39 (s, 5H), 2.08-2.25 (m, 9H), 1.81-1.94 (m, 4H), 1.70-1.78 (m, 2H), 1.49-1.69 (m, 6H), 1.34-1.44 (m, 3H), 1.16-1.32 (m, 10H), 1.02-1.15 (m, 3H), 0.87 (t, J=7.27 Hz, 4H), LCMS: 95.61%; ESI MS m/z calcd. For C₆₀H₇0ClFN₁₀O₇ ([M+H]⁺) 1097.73; found 550.5 (half-M/Z); HPLC: 95.03%.

### Example S49. Preparation of Synthesis of N-((1r,4r)-4-((3-chloro-4-isocyanophenyl)(methyl)amino)cyclohexyl)-6-(4-(6-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)-6-oxohexyl)piperidin-1-yl)pyridazine-3-carboxamide (Compound No. 49)

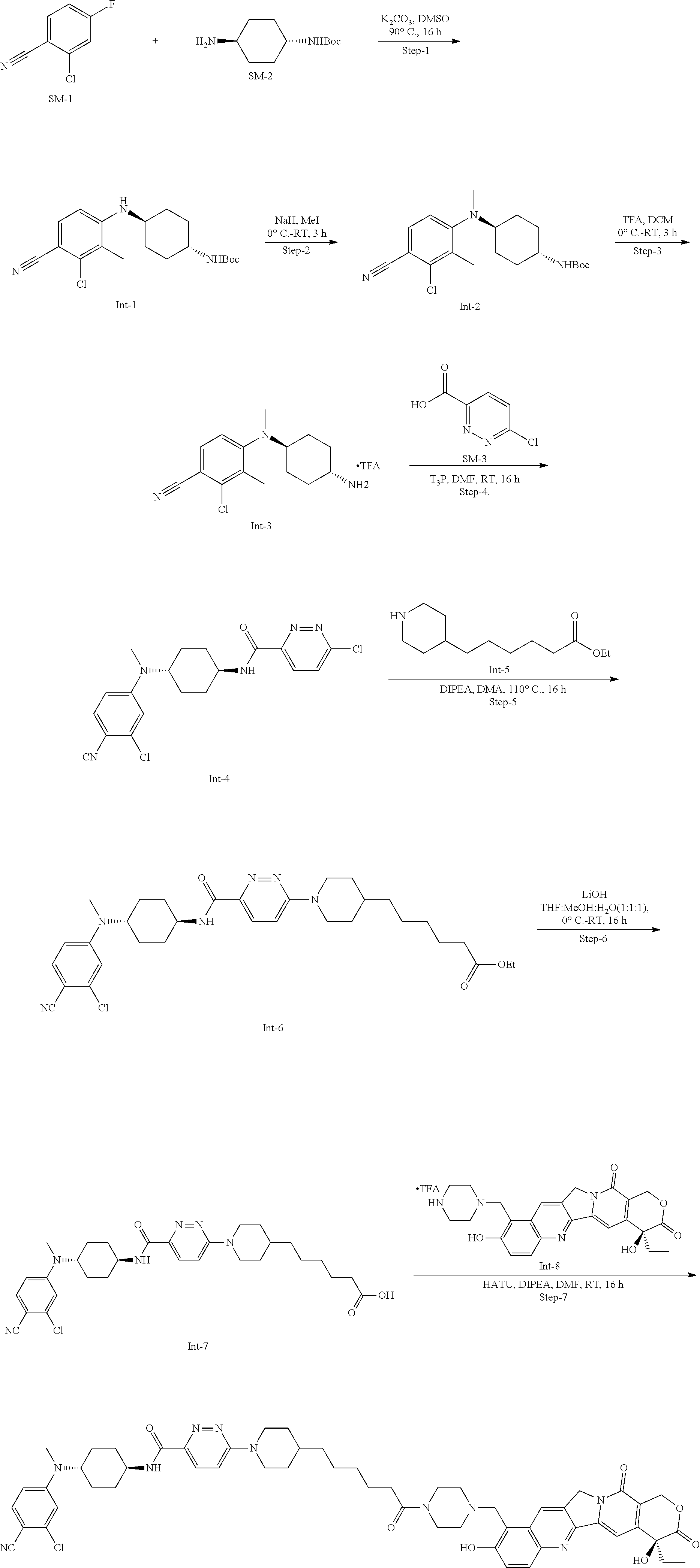

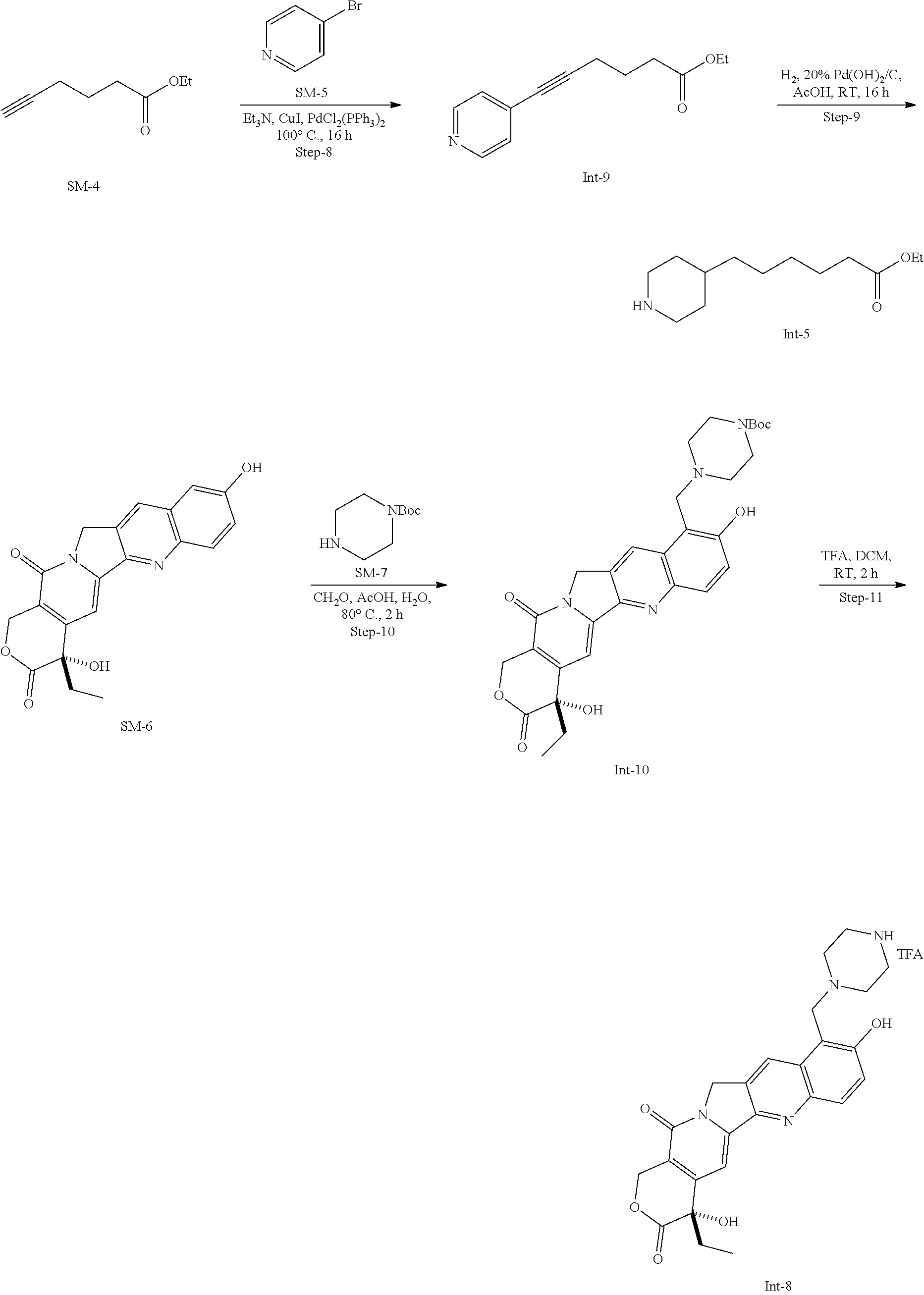

### Step-1: Synthesis of tert-butyl ((1r,4r)-4-((3-chloro-4-cyanophenyl)amino)cyclohexyl) carbamate (Int-1)

To a stirred solution of 2-chloro-4-fluorobenzonitrile (SM-1, 20 g, 128 mmol, 1.0 eq) in DMSO (200 mL) were added tert-butyl ((1r,4r)-4-aminocyclohexyl)carbamate (SM-2, 27.5 g, 128 mmol, 1.0 eq) and K₂CO₃ (35.3 g, 256 mmol, 2 eq) at room temperature. The resultant reaction mixture was heated to 90° C. for 16 h. Progress of the reaction was monitored by TLC. After complete consumption of the starting materials, the reaction mixture was diluted with ice cold water (1 L) and stirred for 10 min at room temperature. The obtained solids were filtered and washed with water, dried under vacuum. The crude solid compound was recrystallized by using EtOH to afford tert-butyl ((1r,4r)-4-((3-chloro-4-cyanophenyl)amino)cyclohexyl)carbamate (Int-1, 30 g, 67%) as an off white solid.

### Step-2: Synthesis of tert-butyl ((1r,4r)-4-((3-chloro-4-cyanophenyl)(methyl)amino) cyclohexyl) carbamate (Int-2)

To a stirred solution of tert-butyl ((1r,4r)-4-((3-chloro-4-cyanophenyl)amino)cyclohexyl) carbamate (Int-1, 30 g, 57.1 mmol, 1.0 eq) in DMF (300 mL) was added NaH (2.96 g, 74.3 mmol, 1.3 eq) under nitrogen atmosphere at 0° C. The resultant reaction mixture was allowed to room temperature and stirred for 30 min. then added methyl iodide (4.6 mL, 74.3 mmol, 1.3 eq) drop wise at 0° C. and allowed to room temperature, stirred for 3 h. Progress of the reaction was monitored by TLC. After complete consumption of starting material, the reaction mixture was diluted with ice cold water (1 L) and stirred for 10 min at room temperature. The obtained solids were filtered and washed with water, dried under vacuum. The obtained crude solid compound was recrystallized by using EtOH to afford tert-butyl ((1r,4r)-4-((3-chloro-4-cyanophenyl)(methyl) amino) cyclohexyl) carbamate (Int-2, 20 g, 64%) as an off white solid.

### Step-3: Synthesis of 4-(((1r,4r)-4-aminocyclohexyl)(methyl)amino)-2-chlorobenzonitrile. TFA Salt (Int-3)

To a stirred solution of tert-butyl ((1r,4r)-4-((3-chloro-4-cyanophenyl)(methyl)amino) cyclohexyl)carbamate (Int-2, 20 g, 54.9 mmol, 1.0 eq) in DCM (300 mL) was added TFA (100 mL, 5 vol) at 0° C. under nitrogen atmosphere. The resultant reaction mixture was allowed to room temperature and stirred for 2 h. Progress of the reaction was monitored by TLC. After complete consumption of starting material, the volatiles were evaporated under reduced pressure to obtain crude compound, which was washed with diethyl ether (20 mL) and dried under vacuum to afford 4-(((1r,4r)-4-aminocyclohexyl)(methyl)amino)-2-chlorobenzonitrile (Int-3, 15 g, crude) as an off white solid.

### Step-4: Synthesis of 6-chloro-N-((1r,4r)-4-((3-chloro-4-cyanophenyl)(methyl)amino) cyclohexyl)pyridazine-3-carboxamide (Int-4)

To a stirred solution of 4-(((1r,4r)-4-aminocyclohexyl)(methyl)amino)-2-chlorobenzonitrile. TFA salt (Int-3, 15 g, 57.03 mmol, 1.0 eq) and 6-chloropyridazine-3-carboxylic acid (SM-3, 9.04 g, 57.03 mmol, 1.0 eq) in DMF (150 mL) were added T3P (36 g, 114 mmol, 2 eq) and DIPEA (30 mL, 171 mmol, 3 eq) at room temperature and stirred for 16 h. Progress of the reaction was monitored by TLC. After complete consumption of the starting materials, the reaction mixture was diluted with ice cold water (1 L) and stirred for 10 min at room temperature, the obtained solids were filtered and washed with water, dried under vacuum to afford 6-chloro-N-((1r,4r)-4-((3-chloro-4-cyanophenyl)(methyl)amino)cyclohexyl)pyridazine-3-carboxamide (Int-4, 13 g, 56%) as an off white solid.

### Step-5: Synthesis of ethyl 6-[1-[6-[[4-(3-chloro-4-cyano-N-methyl-anilino) cyclohexyl] carbamoyl]pyridazin-3-yl]-4-piperidyl]hexanoate (Int-6)

To a stirred solution of 6-chloro-N-((1r,4r)-4-((3-chloro-4-cyanophenyl)(methyl)amino) cyclohexyl)pyridazine-3-carboxamide (Int-4, 3.0 g, 7.42 mmol, 1.0 eq) in DMA (30 mL) were added ethyl 6-(4-piperidyl)hexanoate (Int-5, 2.5 g, 11.1 mmol, 1.5 eq) and DIPEA (1.1 g, 6 mL, 37.1 mmol, 5 eq) at room temperature. The resultant reaction mixture was heated to 110° C. and stirred for 16 h. Progress of the reaction was monitored by TLC. After complete consumption of the starting materials the reaction mixture was diluted with ice cold water (100 mL) and extracted with ethyl acetate (2×150 mL). The combined organic layer was washed with water (200 mL), brine (200 mL) and dried over anhydrous sodium sulphate, filtered, and concentrated under reduced pressure to obtain crude compound, which was purified by combi flash column by eluting with 80% ethyl acetate in henxane to afford 6-[1-[6-[[4-(3-chloro-4-cyano-N-methyl-anilino) cyclohexyl]carbamoyl]pyridazin-3-yl]-4-piperidyl] hexanoate (Int-6, 1.7 g, 38%) as an off white solid.

### Step-6: Synthesis of 6-[1-[6-[[4-(3-chloro-4-cyano-N-methyl-anilino) cyclohexyl] carbamoyl]pyridazin-3-yl]-4-piperidyl] hexanoic acid (Int-7)

To a stirred solution of ethyl 6-[1-[6-[[4-(3-chloro-4-cyano-N-methyl-anilino) cyclohexyl] carbamoyl] pyridazin-3-yl]-4-piperidyl] hexanoate (Int-6, 1.7 g, 2.85 mmol, 1.0 eq) in THF (20 mL). MeOH (20 mL) and H₂O (20 mL) was added LiOH·H₂O (1.19 g, 28.5 mmol, 10 eq) portion wise at 0° C. The resultant reaction mixture was allowed to room temperature and stirred for 16 h. Progress of the reaction was monitored by TLC. After complete consumption of the starting materials, the volatiles. THF and MeOH were evaporated under reduced pressure to afford crude compound, which was diluted with water (50 mL) and adjusted the P^{H} to 5 by using 1N HCl, the obtained solids were filtered and washed with water. Et₂O, dried under vacuum to afford 6-[1-[6-[[4-(3-chloro-4-cyano-N-methyl-anilino) cyclohexyl]carbamoyl] pyridazin-3-yl]-4-piperidyl] hexanoic acid (Int-7, 1.4 g, 86%) as a white solid.

### Step-7: Synthesis of N-((1r,4r)-4-((3-chloro-4-isocyanophenyl)(methyl)amino)cyclohexyl)-6-(4-(6-(4-(((S)-4-ethyl-4,9-dihydroxy-3,14-dioxo-3,4,12,14-tetrahydro-1H-pyrano [3′,4′:6,7]indolizino[1,2-b]quinolin-10-yl)methyl)piperazin-1-yl)-6-oxohexyl)piperidin-1-yl)pyridazine-3-carboxamide

To a stirred solution of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione (Int-8, 150 mg, 0.32 mmol, 1 eq) in DMF (5 mL) were added DIPEA (0.17 mL, 0.95 mmol, 3 eq), HATU (180 mg, 0.48 mmol, 1.2 eq) and 6-(1-(6-(((1r,4r)-4-((3-chloro-4-isocyanophenyl)(methyl)amino)cyclohexyl)carbamoyl)pyridazin-3-yl)piperidin-4-yl)hexanoic acid (Int-7, 180 mg, 0.32 mmol, 1 eq) at room temperature. The resultant reaction mixture was stirred at room temperature for 16 h. Progress of the reaction was monitored by TLC. After complete consumption of starting materials, the reaction mixture was poured into ice cold water (50 ml) and extracted with 10% MeOH in DCM. The combined organic layer was washed with water (50 ml), brine (50 mL) and dried over anhydrous Na₂SO₄, filtered and concentrated under reduced pressure to afford crude compound, which was purified by reverse phase prep. HPLC (by eluting with Mobile phase A: 0.1% NH₄HCO₃ in water and Mobile phase B: Acetonitrile) to afford N-((1r,4r)-4-((3-chloro-4-isocyanophenyl)(methyl)amino)cyclohexyl)-6-(4-(6-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)-6-oxohexyl)piperidin-1-yl)pyridazine-3-carboxamide, (44 mg, 14%) as an off white solid.

¹H NMR (400 MHz, DMSO-d₆): δ ppm 8.76-8.79 (m, 1H), 8.47 (d, J=8.38 Hz, 1H), 8.16-8.18 (m, 1H), 7.99 (d, J=9.26 Hz, 1H), 7.77-7.81 (m, 1H), 7.60 (d, J=9.01 Hz, 1H), 7.44-7.49 (m, 1H), 7.32 (d, J=9.76 Hz, 1H), 7.24-7.27 (m, 1H), 6.94 (d, J=2.38 Hz, 1H), 6.80-6.84 (m, 1H), 6.47-6.54 (m, 1H), 5.75-5.76 (m, 1H), 5.41 (s, 2H), 5.26 (s, 2H), 4.43-4.51 (m, 2H), 4.02 (s, 2H), 3.73-3.89 (m, 2H), 3.44 (s, 4H), 2.96 (t, J=11.69 Hz, 3H), 2.85 (s, 3H), 2.29 (t, J=7.38 Hz, 2H), 1.82-1.95 (m, 4H), 1.61-1.80 (m, 9H), 1.46-1.52 (m, 4H), 1.19-1.35 (m, 6H), 1.07-1.13 (m, 2H), 0.88 (t, J=7.32 Hz, 3H); ESI MS m/z calcd. For C₅₅H₆₃ClN₁₀₀₇ ([M+H]⁺) 1011.46; found 1011.4.

### Step-8: Synthesis of ethyl 6-(4-pyridyl)hex-5-ynoate (Int-9)

To a stirred solution of ethyl hex-5-ynoate (SM-4, 4 g, 28.5 mmol, 1 eq) in Et₃N (80 mL) was added 4-bromopyridine (SM-5, 4.95 g, 31.3 mmol, 1.1 eq) at room temperature. The resultant mixture was degassed with Argon for 10 min, after that Copper (I) iodide (54 mg, 0.28 mmol, 0.01 eq) and PdCl₂ (PPh₃)₂ (400 mg, 0.57 mmol, 0.02 eq) were added at room temperature. The resultant reaction mixture was stirred at 100° C. for 16 h. Progress of the reaction was monitored by TLC. After complete consumption of starting materials, the reaction mixture cooled to room temperature and diluted with EtOAc (200 mL), filtered through celite bed. Filtrate was washed with water (100 mL), dried over anhydrous Na₂SO₄, and concentrated under reduced pressure to afford ethyl 6-(4-pyridyl) hex-5-ynoate (Int-9, 5.5 g, 89%) as a yellow liquid.

### Step-9: Synthesis of ethyl 6-(4-piperidyl)hexanoate (Int-5)

To a stirred solution of ethyl 6-(4-pyridyl)hex-5-ynoate (Int-9, 5.5 g, 25.3 mmol, 1.0 eq) in Acetic acid (165 mL) was added 20% Pd(OH)₂ (2.26 g) at room temperature. The resultant reaction mixture was stirred at room temperature for 16 h under H₂ atmosphere (100 psi). Progress of the reaction was monitored by TLC. After complete consumption of starting materials, the reaction mixture was filtered through celite bed and washed with 10% MeOH in DCM. The obtained filtrate concentrated under reduced pressure to obtain ethyl 6-(4-piperidyl)hexanoate (Int-5, 3 g, 53%) as a brown colour liquid.

### Step-10: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-10)

To a stirred solution of (S)-4-ethyl-4,9-dihydroxy-1,12-dihydro-14H-pyrano[3′,4′:6,7]indolizino[1,2-b]quinoline-3,14(4H)-dione·HCl salt (SM-6, 2.5 g, 6.86 mmol, 1.0 eq) and tert-butyl piperazine-1-carboxylate (SM-7, 1.85 g, 10.3 mmol, 1.5 eq) in acetic acid (10 mL) under inert atmosphere was added 37% solution of formaldehyde (0.29 mL, 8.24 mmol, 1.2 eq) at room temperature. The resultant reaction mixture was heated to 80° C. and stirred for 2 h in a sealed tube. Progress of the reaction was monitored by TLC. After complete consumption of starting materials, the volatiles were evaporated under reduced pressure to obtain crude compound, which was basified with aq. ammonia until the pH reached to 9, filtered the precipitated solid and washed with water (10 mL) and dried under vacuum to afford tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-10, 1.9 g, 50%) as a yellow solid.

### Step-11: Synthesis of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFAsalt (Int-8)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1carboxylate (Int-10, 2 g, 3.5 mmol, 1.0 eq) in DCM (20 mL) under nitrogen atmosphere was added TFA (2.7 mL, 35 mmol, 10 eq) at 0° C. The resultant reaction mixture was warmed to room temperature and stirred for 2 h. Progress of the reaction was monitored by TLC. After complete consumption of starting materials, the volatiles were evaporated under reduced pressure to obtain crude compound, which was washed with diethyl ether (10 mL) and dried under vacuum to afford(S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione (Int-8, 1.4 g, 83%) as a yellow solid.

### Example S50. Preparation of N-((1r,4r)-4-(3-chloro-4-isocyano-2-methylphenoxy)cyclohexyl)-6-(4-(6-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)-6-oxohexyl)piperidin-1-yl)pyridazine-3-carboxamide (Compound No. 50)

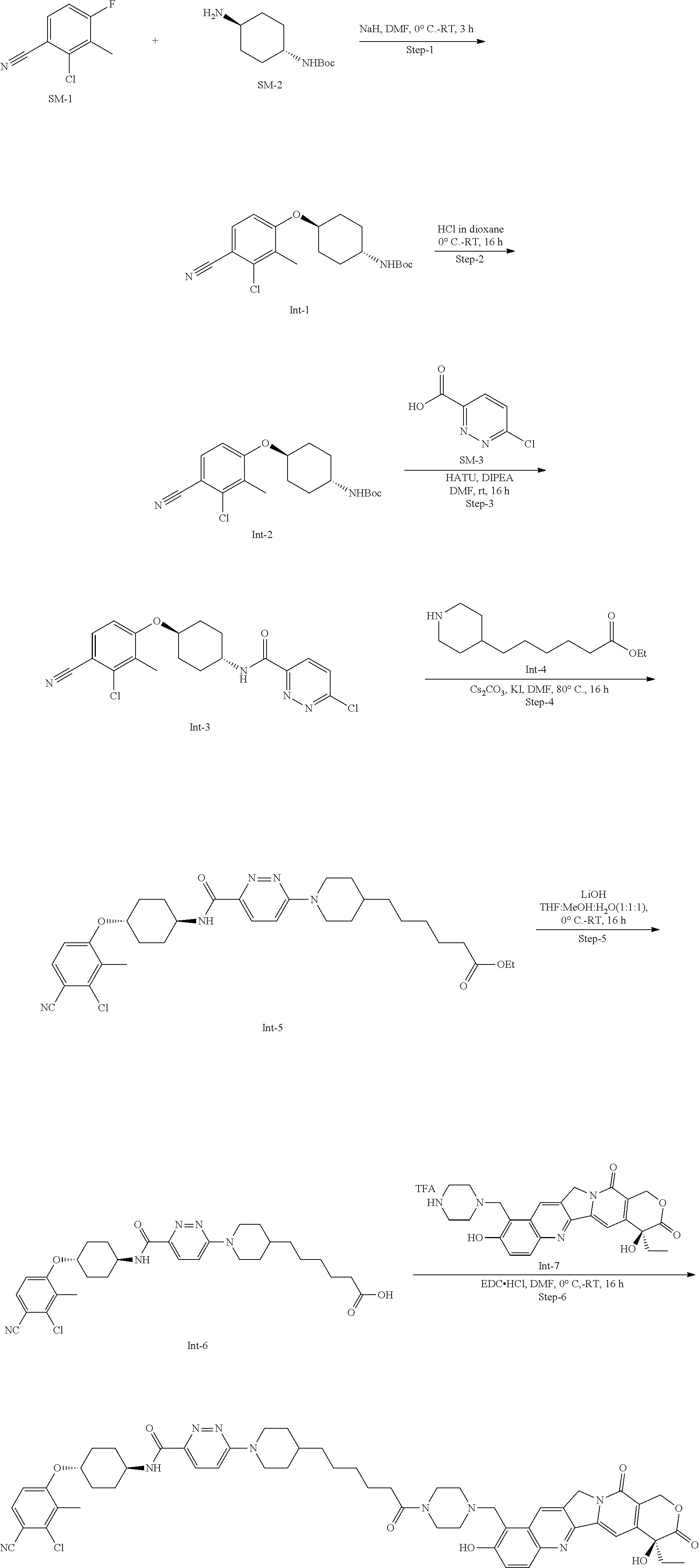

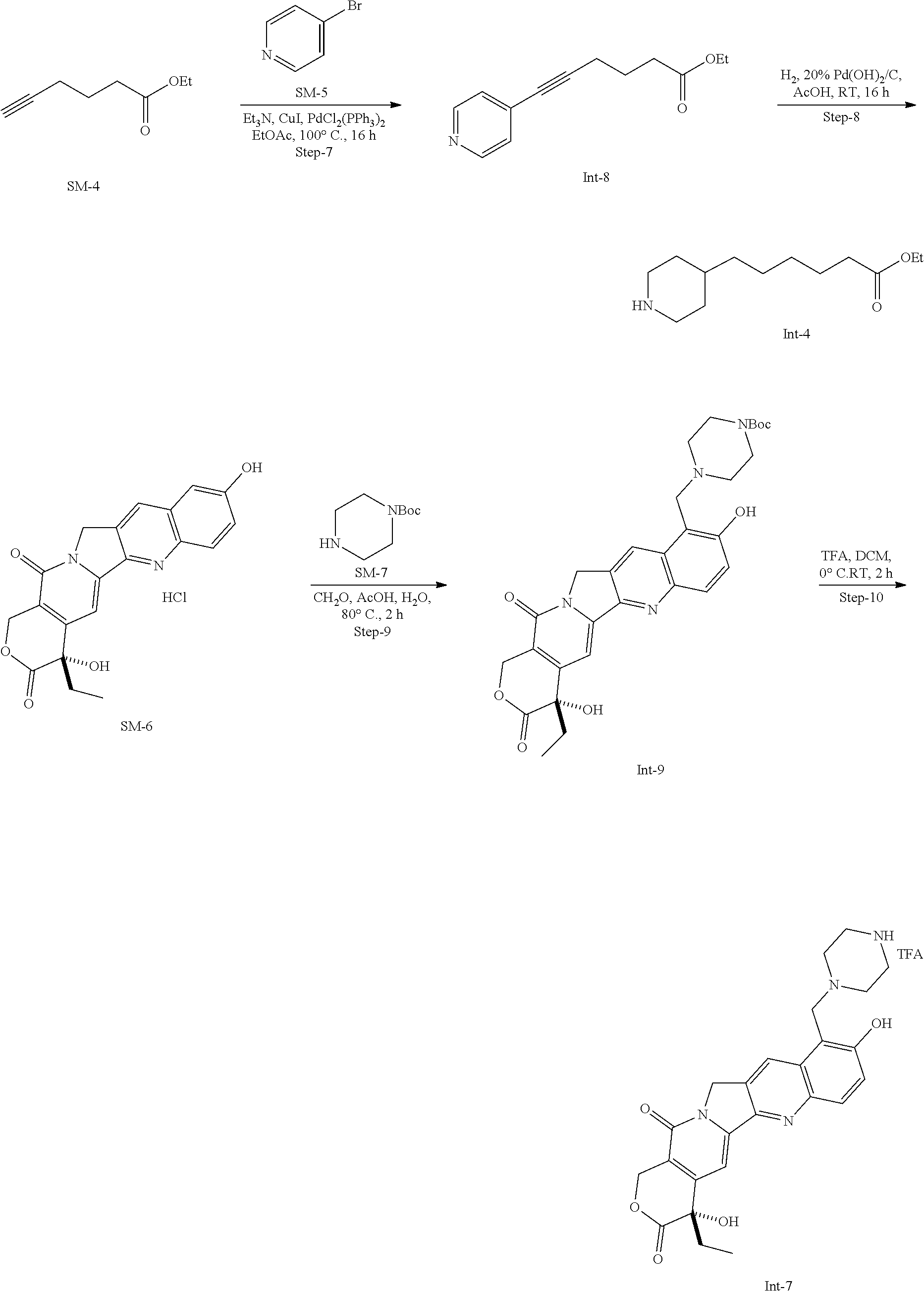

### Step-1: Synthesis of tert-butyl ((1r,4r)-4-(3-chloro-4-cyano-2-methylphenoxy) cyclohexyl)carbamate (Int-1)

To a stirred solution of tert-butyl ((1r,4r)-4-hydroxycyclohexyl)carbamate, (SM-2, 10 g, 46.5 mmol) in DMF (100 mL) under nitrogen atmosphere was added NaH (4.08 g, 102 mmol) at 0° C. and warmed to room temperature, stirred for 30 min. And then 2-chloro-4-fluoro-3-methylbenzonitrile (SM-1, 7.86 g, 46.51 mmol) was added portion wise over a period of 10 min at room temperature and stirred for 3 h. Progress of the reaction was monitored by TLC. After complete consumption of starting materials, the reaction mixture was quenched with ice cold water (300 mL) and the precipitated solid was filtered and dried under vacuum to afford tert-butyl ((1r,4r)-4-(3-chloro-4-cyano-2-methyl phenoxy)cyclohexyl)carbamate (Int-1, 15 g, 88%) as an off-white solid.

### Step-2: Synthesis of 4-(((1r,4r)-4-aminocyclohexyl)oxy)-2-chloro-3-methylbenzonitrile hydrochloride (Int-2)

To a stirred solution of tert-butyl ((1r,4r)-4-(3-chloro-4-cyano-2-methylphenoxy) cyclohexyl)carbamate (Int-1, 15 g, 41 mmol) was added 4M HCl in 1,4-dioxane (75 mL) at 0° C. and stirred for 16 h at room temperature. Progress of the reaction was monitored by TLC. After complete consumption of starting materials, the volatiles were evaporated under reduced pressure and washed with diethyl ether, dried under vacuum to afford 4-(((1r,4r)-4-aminocyclohexyl)oxy)-2-chloro-3-methylbenzonitrile hydrochloride (Int-2, 15 g, crude) as an off-white solid.

### Step-3: Synthesis of 6-chloro-N-((1r,4r)-4-(3-chloro-4-cyano-2-methylphenoxy) cyclohexyl)pyridazine-3-carboxamide (Int-3)

To the mixture of 4-(((1r,4r)-4-aminocyclohexyl)oxy)-2-chloro-3-methyl benzonitrilo hydrochloride (Int-2, 15 g, 56.9 mmol) and 6-chloropyridazine-3-carboxylic acid (SM-3, 9 g, 56.9 mmol) in DMF (100 mL) were added HATU (32.4 g, 85.44 mmol) and DIPEA (49 mL, 284 mmol) at 0° C. and the resultant reaction mixture was stirred at room temperature for 16 h. Progress of the reaction was monitored by TLC. After complete consumption of starting materials, volatiles were evaporated under reduced pressure and the obtained crude material was diluted with water (500 mL) and extracted with ethyl acetate (3×300 mL). The combined organic extracts were washed with water (400 mL) and brine (400 mL), dried over anhydrous sodium sulphate, filtered, and concentrated under reduced pressure to afford crude compound, which was purified by column chromatography by eluting with 60-80% ethyl acetate in n-heptane to afford 6-chloro-N-((1r,4r)-4-(3-chloro-4-cyano-2-methylphenoxy)cyclohexyl) pyridazine-3-carboxamide (Int-3, 6 g, 27%) as an off-white solid.

### Step-4: Synthesis of ethyl 6-(1-(6-(((1r,4r)-4-(3-chloro-4-isocyano-2-methylphenoxy) cyclohexyl)carbamoyl)pyridazin-3-yl)piperidin-4-yl)hexanoate (Int-5)

To a stirred solution of 6-chloro-N-((1r,4r)-4-(3-chloro-4-cyano-2-methylphenoxy)cyclohexyl)pyridazine-3-carboxamide (Int-3, 1.07 g, 2.64 mmol, 1.0 eq) in DMF (10 mL) was added ethyl 6-(piperidin-4-yl)hexanoate (Int-4, 600 mg, 2.6 mmol, 1.0 eq) and Cs₂CO₃ (2.5 g, 7.7 mmol, 3 eq) and KI (220 mg, 1.32 mmol) at room temperature. The reaction mixture was heated to 80° C. and stirred for 16 h. Progress of the reaction was monitored by TLC. After complete consumption of starting materials, the reaction mixture was diluted with cold water (100 mL) and extracted with ethyl acetate (2×150 mL). The combined organic layer was washed with water (200 mL), brine (200 mL) and dried over anhydrous sodium sulphate, filtered, and concentrated under reduced pressure to obtain crude compound, which was purified by combi flash column by eluting with 80% ethyl acetate in heptane to afford ethyl 6-(1-(6-(((1r,4r)-4-(3-chloro-4-isocyano-2-methylphenoxy)cyclohexyl)carbamoyl)pyridazin-3-yl)piperidin-4-yl)hexanoate (Int-5, 300 mg, 19%) as a pale brown solid.

### Step-5: Synthesis of 6-(1-(6-(((1r,4r)-4-(3-chloro-4-isocyano-2-methylphenoxy) cyclohexyl)carbamoyl)pyridazin-3-yl)piperidin-4-yl)hexanoic acid (Int-6)

To a stirred solution of ethyl 6-(1-(6-(((1r,4r)-4-(3-chloro-4-isocyano-2-methylphenoxy)cyclohexyl)carbamoyl)pyridazin-3-yl)piperidin-4-yl)hexanoate (Int-5, 50 mg, 0.08 mmol, 1.0 eq) in THF (0.5 mL). McOH (0.5 mL). H₂O (0.5 mL) was added LiOH·H₂O (6.5 mg, 0.26 mmol, 3.1 eq) portion wise at 0° C. The resultant reaction mixture was allowed to room temperature and stirred for 16 h. Progress of the reaction was monitored by TLC. After complete consumption of the starting materials, the volatiles (THF and MeOH) were distilled under reduced pressure, followed by water (50 mL) was added and adjusted the P^{H} to 5 by using 1N HCl solution and extracted with ethyl acetate (2×15 mL). The combined organic layer was washed with water (10 mL), brine (10 mL) and dried over anhydrous sodium sulphate, filtered, and concentrated under reduced pressure to obtain crude compound, which was washed with water, Et₂O and dried under vacuum to afforded 6-(1-(6-(((1r,4r)-4-(3-chloro-4-isocyano-2-methylphenoxy)cyclohexyl)carbamoyl)pyridazine-3-yl)piperidin-4-yl)hexanoic acid (Int-6, 40 mg, 83%) as a yellow solid.

### Step-6: Synthesis of N-((1r,4r)-4-(3-chloro-4-isocyano-2-methylphenoxy)cyclohexyl)-6-(4-(6-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)-6-oxohexyl)piperidin-1-yl)pyridazine-3-carboxamide

To a stirred solution of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFA salt (Int-7, 211 mg, 0.45 mmol, 1 eq) in DMF (5 mL) were added 6-(1-(6-(((1r,4r)-4-(3-chloro-4-isocyano-2-methylphenoxy)cyclohexyl)carbamoyl)pyridazin-3-yl)piperidin-4-yl)hexanoic acid (Int-6, 260 mg, 0.45 mmol), DIPEA (0.25 mL, 1.36 mmol, 3 eq), EDC·HCl (106 mg . . . 0.54 mmol, 1.2 eq) and DMAP (28 mg, 0.22 mmol) at room temperature. The resultant reaction mixture was stirred at room temperature for 16 h. Progress of the reaction was monitored by TLC. After complete consumption of starting materials, the reaction mixture was poured into ice cold water (50 ml) and extracted with 10% MeOH in DCM. The combined organic layer was washed with water (50 ml), brine (50 mL) and dried over anhydrous Na₂SO₄, filtered and concentrated under reduced pressure to afford crude compound, which was purified by reverse phase prep. HPLC purification (by eluting with Mobile phase A: 0.1% NH₄HCO₃ in water and Mobile phase B: Acetonitrile) to afford the title compound (90 mg, 19.4%) as an off white solid.

¹H NMR (400 MHz, DMSO-d₆): δ ppm 8.75-8.79 (m, 1H), 8.56 (d, J=8.25 Hz, 1H), 7.96-8.01 (m, 1H), 7.74-7.81 (m, 2H), 7.47 (d, J=9.26 Hz, 1H), 7.23-7.35 (m, 3H), 6.45-6.51 (m, 1H), 5.41 (s, 2H), 5.25 (s, 2H), 4.42-4.55 (m, 3H), 4.14 (dd, J=5.44, 3.44 Hz, 1H), 4.02 (s, 2H), 3.82-3.94 (m, 1H), 3.44 (s, 4H), 2.90-3.01 (m, 2H), 2.26-2.34 (m, 2H), 2.24 (s, 3H), 2.07-2.16 (m, 2H), 1.81-1.95 (m, 5H), 1.76 (d, J=11.38 Hz, 2H), 1.44-1.69 (m, 8H), 1.17-1.38 (m, 8H), 1.03-1.16 (m, 2H), 0.88 (t, J=7.32 Hz, 3H); ESI MS m/z calcd. For C₅₅H₆₂ClN₉O₈ ([M+H]⁺) 1012.44; found 1012.4.

### Step-7: Synthesis of ethyl 6-(4-pyridyl)hex-5-ynoate (Int-8)

To a stirred solution of ethyl hex-5-ynoate (SM-4, 4 g, 28.5 mmol, 1 eq) in Et₃N (80 mL) was added 4-bromopyridine (SM-5, 4.95 g, 31.3 mmol, 1.1 eq) at room temperature and degassed with Argon for 10 min, after that Copper (I) iodide (54 mg, 0.28 mmol, 0.01 eq), PdCl₂ (PPh₃)₂ (400 mg, 0.57 mmol, 0.02 eq) were added at room temperature. The resultant reaction mixture was stirred at 100° C. for 16 h. Progress of the reaction was monitored by TLC. After complete consumption of starting materials the reaction mixture was cooled to room temperature and diluted with EtOAc (200 mL), filtered through celite bed. Filtrate was washed with water (100 mL), brine (100 mL) and dried over anhydrous Na₂SO₄, filtered and concentrated under reduced pressure to afford ethyl 6-(4-pyridyl) hex-5-ynoate (Int-8, 5.5 g, 89%) as a yellow liquid.

### Step-8: Synthesis of ethyl 6-(4-piperidyl)hexanoate (Int-4)

To a stirred solution of ethyl 6-(4-pyridyl)hex-5-ynoate (Int-8, 5.5 g, 25.3 mmol, 1 eq) in Acetic acid (165 mL) was added 20% Pd(OH) 2 (2.26 g) at room temperature and the resultant reaction mixture was stirred at room temperature for 16 h. under H₂ pressure (100 psi). The progress of the reaction was monitored by TLC. After complete consumption of starting materials the reaction mixture was filtered through celite bed and washed with 10% MeOH in DCM, the obtained filtrate was concentrated under reduced pressure to obtain crude ethyl 6-(4-piperidyl)hexanoate (Int-4, 3 g, 53%) as a brown colour liquid.

### Step-9: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-9)

To a stirred solution of (S)-4-ethyl-4,9-dihydroxy-1,12-dihydro-14H-pyrano[3′,4′:6,7]indolizino[1,2-b]quinoline-3,14(4H)-dione·HCl salt (SM-6, 2.5 g, 6.86 mmol, 1.0 eq) and tert-butyl piperazine-1-carboxylate (SM-7, 1.85 g, 10.3 mmol, 1.5 eq) in acetic acid (10 mL) under inert atmosphere was added 37% solution of formaldehyde (0.29 mL, 8.24 mmol, 1.2 eq) at room temperature. The resultant reaction mixture was heated to 80° C. and stirred for 2 h in a sealed tube. Progress of the reaction was monitored by TLC. After complete consumption of starting materials, the volatiles were evaporated under reduced pressure to obtain crude compound, which was basified with aq. ammonia until the pH reached to 9, filtered the precipitated solid and washed with water (10 mL) and dried under vacuum to afford tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-9, 1.9 g, 50%) as a yellow solid.

### Step-10: Synthesis of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFAsalt (Int-7)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1carboxylate (Int-9, 2 g, 3.5 mmol, 1.0 eq) in DCM (20 mL) under nitrogen atmosphere was added TFA (2.7 mL, 35 mmol, 10 eq) at 0° C., The resultant reaction mixture was warmed to room temperature and stirred for 2 h. Progress of the reaction was monitored by TLC. After complete consumption of starting materials, the volatiles were evaporated under reduced pressure to obtain crude compound, which was washed with diethyl ether (10 mL) and dried under vacuum to afford(S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione (Int-7, 1.4 g, 83%) as a yellow solid.

### Example S51. Preparation of (8S,11R,13S,14S,17R)-17-acetyl-11-(4-((2-(4-((4-((((1S,9S)-9-ethyl-5-fluoro-9-hydroxy-4-methyl-10,13-dioxo-2,3,9,10,13,15-hexahydro-1H,12H-benzo[d]pyrano[3′,4′:6,7]indolizino[1,2-b]quinolin-1-yl)amino)methyl)piperidin-1-yl)methyl)piperidin-1-yl)-2-oxoethyl)(methyl)amino)phenyl)-13-methyl-3-oxo-(Compound No. 51)

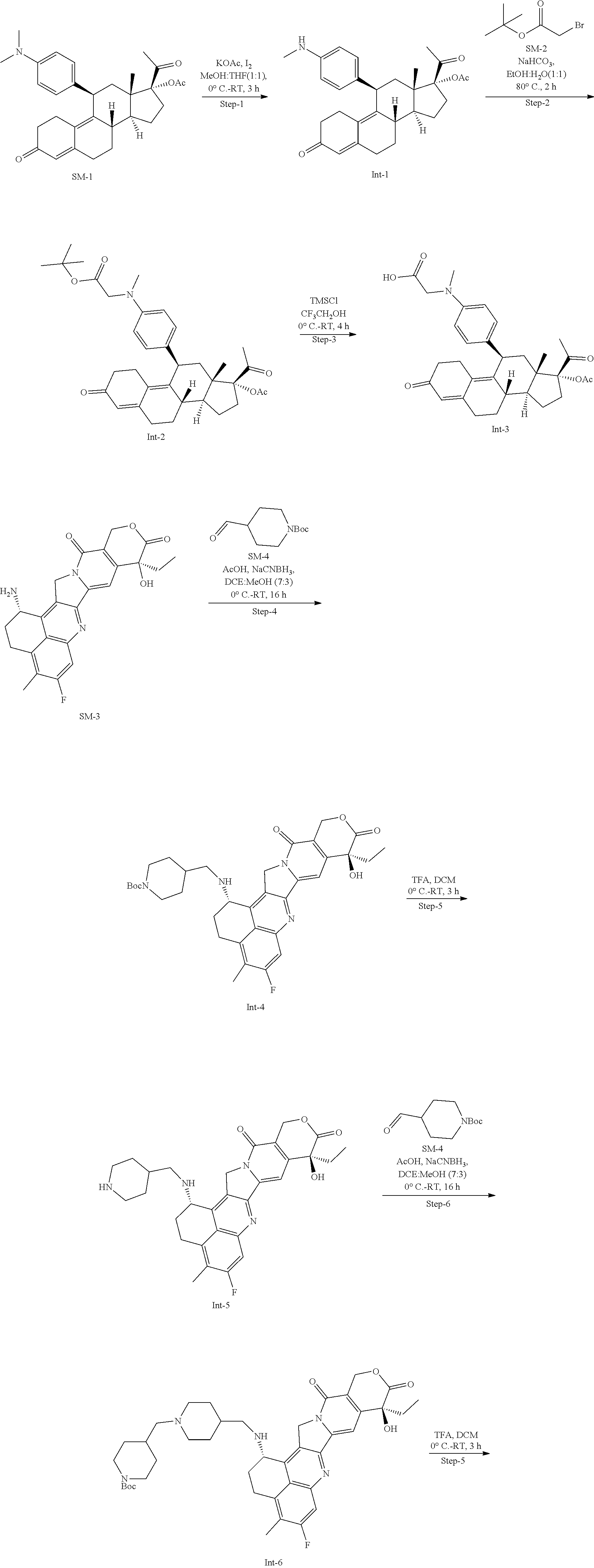

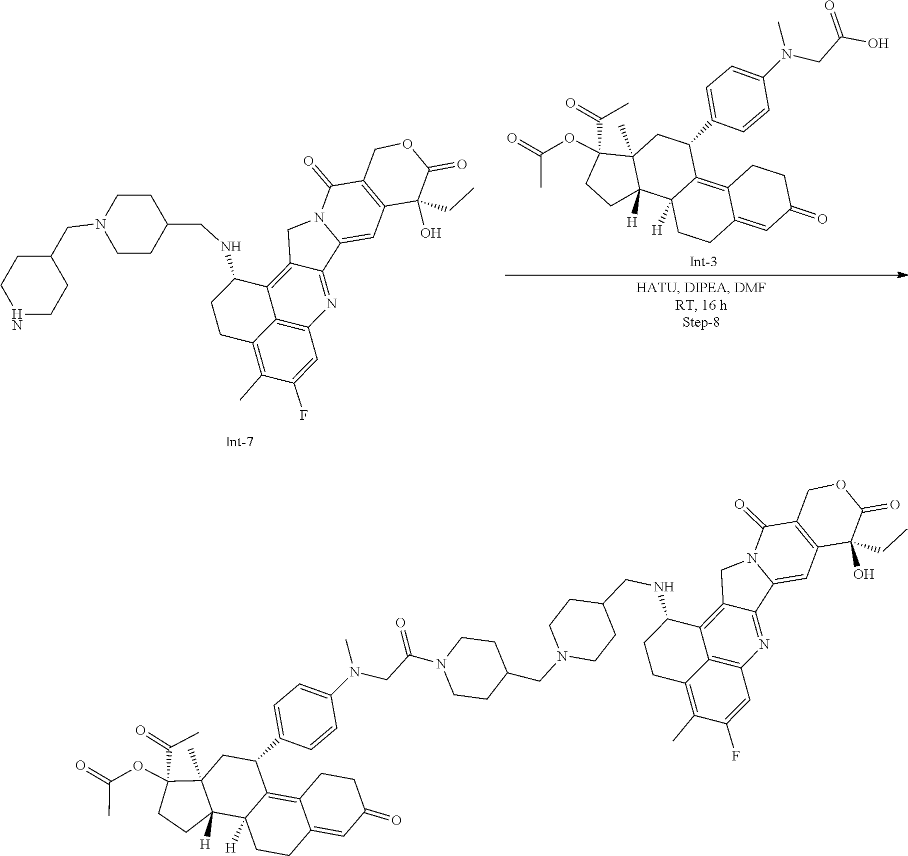 [SMILES: CC[C@@]1(O)C(=O)OCc2c1cc1n(c2=O)Cc2c-1nc1cc(F)c(C)c3c1c2[C@@H](NCC1CCN(CC2CCNCC2)CC1)CC3 ; CC(=O)O[C@]1(C(C)=O)CC[C@H]2[C@@H]3CCC4=CC(=O)CCC4=C3[C@@H](c3ccc(N(C)CC(=O)O)cc3)C[C@@]21C ; CC[C@@]1(O)C(=O)OCc2c1cc1n(c2=O)Cc2c-1nc1cc(F)c(C)c3c1c2[C@@H](NCC1CCN(CC2CCN(C(=O)CN(C)c4ccc([C@H]5C[C@@]6(C)[C@@H](CC[C@]6(OC(C)=O)C(C)=O)[C@@H]6CCC7=CC(=O)CCC7=C65)cc4)CC2)CC1)CC3]

### Step-1: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-1, 10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The resultant reaction mixture was allowed to room temperature and stirred for 3 h. Progress of the reaction was monitored by TLC. After complete consumption of starting materials, the reaction mixture was quenched with sodium thiosulfate (Na₂S₂O₃) solution (50 g in 30 mL water) and extracted with ethyl acetate (2×200 mL), The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 8.0 g, 82%) as an off-white solid which was used in next step without any further purification.

### Step-2: Synthesis of tert-butyl N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycinate (Int-2)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 1 g, 2.17 mmol, 1 eq) in EtOH:H₂O (20 mL, 1:1) were added (SM-2, 0.32 mL, 2.17 mmol, 1 eq) and NaHCO₃ (911 mg, 10.85 mmol, 5 eq) at room temperature. The resultant reaction mixture was heated to 80° C. and stirred for 2 h. Progress of the reaction was monitored by TLC. After complete consumption of starting materials, the reaction mixture was diluted with cold water (50 mL) and extracted with ethyl acetate (2×50 mL). The combined organic extracts were washed with brine (100 mL) and dried over anhydrous sodium sulphate, filtered, and concentrated under reduced pressure to obtain crude compound, which was triturated with n-heptane (2×30 mL) and dried under vacuum to afford tert-butyl N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycinate (Int-2, 900 mg, 72%) as a pale-yellow solid.

### Step-3: Synthesis of N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycine (Int-3)

To a stirred solution of tert-butyl N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycinate (Int-2, 300 mg, 0.521 mmol, 1 eq) in trifluoro ethanol (5 mL) was added chlorotrimethylsilane (1.31 mL, 10.43 mmol, 20 eq) drop wise at 0° C. The resultant reaction mixture was allowed to room temperature and stirred for 4 h. Progress of the reaction was monitored by TLC. After complete consumption of starting materials, the volatiles were evaporated and washed with n-pentane (2×20 mL), dried under vacuum to afford N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycine (Int-3, 250 mg, 92%) as a pale-yellow solid.

### Step-4: Synthesis of tert-butyl 4-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)methyl)piperidine-1-carboxylate (Int-4)

To a stirred solution of (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (SM-3, 3 g, 6.89 mmol, 1 eq) in DCE (70 mL) and Methanol (30 mL), tert-butyl 4-formylpiperidine-1-carboxylate (SM-4, 1.46 g, 6.89 mmol, 1 eq) and acetic acid (3 ml) were added at 0° C. The resultant reaction mixture was stirred at room temperature for 2 h. After that sodium cyanoborohydride (1.08 g, 17.23 mmol, 2.5 eq) was added to above reaction mixture at 0° C. The resultant reaction mixture was stirred at room temperature for 16 h. Progress of the reaction was monitored by TLC. After complete consumption of starting materials, the reaction mixture was cooled to 0° C. and quenched with sat. NaHCO₃ solution (100 mL) and extracted with DCM (2×100 mL). The combined organic layer was washed with water (100 mL) and brine (100 mL), dried over anhydrous sodium sulphate, filtered and concentrated under reduced pressure to obtain crude product, which was purified by reverse phase purification (by using 0.1% FA in water eluted in 55% Acetonitrile in 0.1% FA in water) to afford tert-butyl 4-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)methyl)piperidine-1-carboxylate (Int-4, 2.4 g, 55%) as a white solid.

### Step-5: Synthesis of (1S,9S)-9-ethyl-5-fluoro-9-hydroxy-4-methyl-1-((piperidin-4-ylmethyl)amino)-1,2,3,9,12,15-hexahydro-10H,13H-benzo[de]pyrano[3′,4′:6,7]indolizino[1,2-b]quinoline-10,13-dione (Int-5)

To a stirred solution of tert-butyl 4-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)methyl)piperidine-1-carboxylate (Int-4, 2.4 g, 3.80 mmol, 1 eq) in dichloromethane (40 mL) was added trifluoro acetic acid (10 mL) at 0° C. and then allowed to stir at room temperature for 3 h. Progress of the reaction was monitored by TLC. After complete consumption of starting material, the reaction mixture was evaporated under reduced pressure and the obtained crude material was washed with diethyl ether and dried under vacuum to afford (1S,9S)-9-ethyl-5-fluoro-9-hydroxy-4-methyl-1-((piperidin-4-ylmethyl)amino)-1,2,3,9,12,15-hexahydro-10H,13H-benzo[de]pyrano[3′,4′:6,7]indolizino[1,2-b]quinoline-10,13-dione (Int-5, 1.80 g, 89%) as an orange solid.

### Step-6: Synthesis of tert-butyl 4-((4-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)methyl)piperidin-1-yl)methyl)piperidine-1-carboxylate (Int-6)

To a stirred solution of ((1S,9S)-9-ethyl-5-fluoro-9-hydroxy-4-methyl-1-((piperidin-4-ylmethyl)amino)-1,2,3,9,12,15-hexahydro-10H,13H-benzo[de]pyrano[3′,4′:6,7]indolizino[1,2-b]quinoline-10,13-dione (Int-5, 1.8 g, 3.20 mmol, 1 eq) in DCE (70 mL) and Methanol (30 mL), tert-butyl 4-formylpiperidine-1-carboxylate (SM-4, 1.0 g, 4.70 mmol, 1.5 eq) and acetic acid (2 ml) were added at 0° C. The resultant reaction mixture was stirred at room temperature for 2 h. After that sodium cyanoborohydride (400 mg, 6.31 mmol, 2 eq) was added to above reaction mixture at 0° C. The resultant reaction mixture was stirred at room temperature for 16 h Progress of the reaction was monitored by TLC. After complete consumption of starting materials, the reaction mixture was cooled to 0° C. and quenched with sat. NaHCO₃ solution (100 mL), extracted with DCM (2×100 mL). The combined organic layer was washed with water (100 mL) and brine (100 mL), dried over anhydrous sodium sulphate, filtered and concentrated under reduced pressure to obtain crude product, which was purified by reverse phase purification (using 0.1% FA in water eluted in 55% Acetonitrile in 0.1% FA in water) to afford tert-butyl 4-((4-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)methyl)piperidin-1-yl)methyl)piperidine-1-carboxylate (Int-6, 1.3 g, 56%) as a white solid.

### Step-7: Synthesis of (1S,9S)-5-fluoro-9-hydroxy-4,9-dimethyl-1-(((1-(piperidin-4-ylmethyl)piperidin-4-yl)methyl)amino)-1,2,3,9,12,15-hexahydro-10H,13H-benzo[de]pyrano[3′,4′:6,7]indolizino[1,2-b]quinoline-10,13-dione (Int-7)

To a stirred solution of tert-butyl 4-((4-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)methyl)piperidin-1-yl)methyl)piperidine-1-carboxylate (Int-6, 1.30 g, 1.78 mmol, 1 eq) in dichloromethane (40 mL) was added trifluoro acetic acid (10 mL) at 0° C. and then allowed to stir at room temperature for 3 h. Progress of the reaction was monitored by TLC. After complete consumption of starting material, the reaction mixture was evaporated under reduced pressure, washed with diethyl ether (50 mL) and dried under vacuum to afford (1S,9S)-5-fluoro-9-hydroxy-4,9-dimethyl-1-(((1-(piperidin-4-ylmethyl)piperidin-4-yl)methyl)amino)-1,2,3,9,12,15-hexahydro-10H,13H-benzo[de]pyrano[3′,4′:6,7]indolizino[1,2-b]quinoline-10,13-dione (Int-7, 1.0 g, 89%) as an orange solid.

### Step-8: Synthesis of (8S,11R,13S,14S,17R)-17-acetyl-11-(4-((2-(4-((4-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)methyl)piperidin-1-yl)methyl)piperidin-1-yl)-2-oxoethyl)(methyl)amino)phenyl)-13-methyl-3-oxo-2,3,6,7,8,11,12,13,14,15,16,17-dodecahydro-1H-cyclopenta[a]phenanthren-17-yl acetate

To a stirred solution of N-(4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)-N-methylglycine (Int-3, 500 mg, 0.75 mmol) in DMF (5 mL). (1S,9S)-5-fluoro-9-hydroxy-4,9-dimethyl-1-(((1-(piperidin-4-ylmethyl)piperidin-4-yl)methyl)amino)-1,2,3,9,12,15-hexahydro-10H,13H-benzo[de]pyrano[3′,4′:6,7]indolizino[1,2-b]quinoline-10,13-dione (Int-7, 380 mg, 0.75 mmol, 1 eq) and N,N-Diisopropylethylamine (0.29 g, 2.25 mmol, 3.0 eq) were added at room temperature and stirred for 5 min. After that 1-Propane phosphonic anhydride (50%) in ethyl acetate (0.95 g, 1.50 mmol, 2 eq) was added at room temperature. The resultant reaction mixture was stirred at room temperature for 16 h. Progress of the reaction was monitored by TLC. After complete consumption of starting material, the reaction mixture was diluted with ice cold water (50 mL) and extracted with EtOAc (2×50 mL). The combined organic layer was washed with brine solution (50 mL), dried over anhydrous sodium sulphate, filtered, and concentrated under reduced pressure to obtain crude compound, which was purified by reverse phase (using 0.1% FA in water eluted in 55% Acetonitrile in 0.1% FA in water) to afford the title compound (68 mg, 8%) as an off white solid.

¹H NMR (400 MHz, DMSO-d₆): δ ppm 7.74 (d, J=11.13 Hz, 1H), 7.30 (s, 1H), 6.95 (br d, J=8.68 Hz, 2H), 6.42-6.58 (m, 3H), 5.67 (s, 1H), 5.34-5.45 (m, 3H), 4.39 (br d, J=7.09 Hz, 1H), 4.03-4.29 (m, 4H), 3.78-3.91 (m, 1H), 3.16-3.23 (m, 1H), 2.85-3.09 (m, 7H), 2.71-2.82 (m, 3H). 2.30-2.39 (m, 5H), 2.02-2.25 (m, 11H), 1.94-2.00 (m, 5H), 1.83-1.93 (m, 4H), 1.62-1.83 (m, 8H), 1.34-1.56 (m, 3H), 1.23 (s, 3H), 1.07 (s, 2H), 0.87 (br t, J=7.34 Hz, 5H), 0.61-0.74 (m, 1H), 0.23 (s, 3H); LCMS: 95.22%; ESI MS m/z calcd. For C₆₇H₇₉FN₆O₉ ([M+H]⁺) 1131.3; found 566.4; HPLC: 95.10%.

### Example S52. Preparation of N-((1r,4r)-4-(3-chloro-4-isocyano-2-methylphenoxy)cyclohexyl)-6-(4-(6-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)hexyl)piperidin-1-yl)pyridazine-3-carboxamide (Compound No. 52)

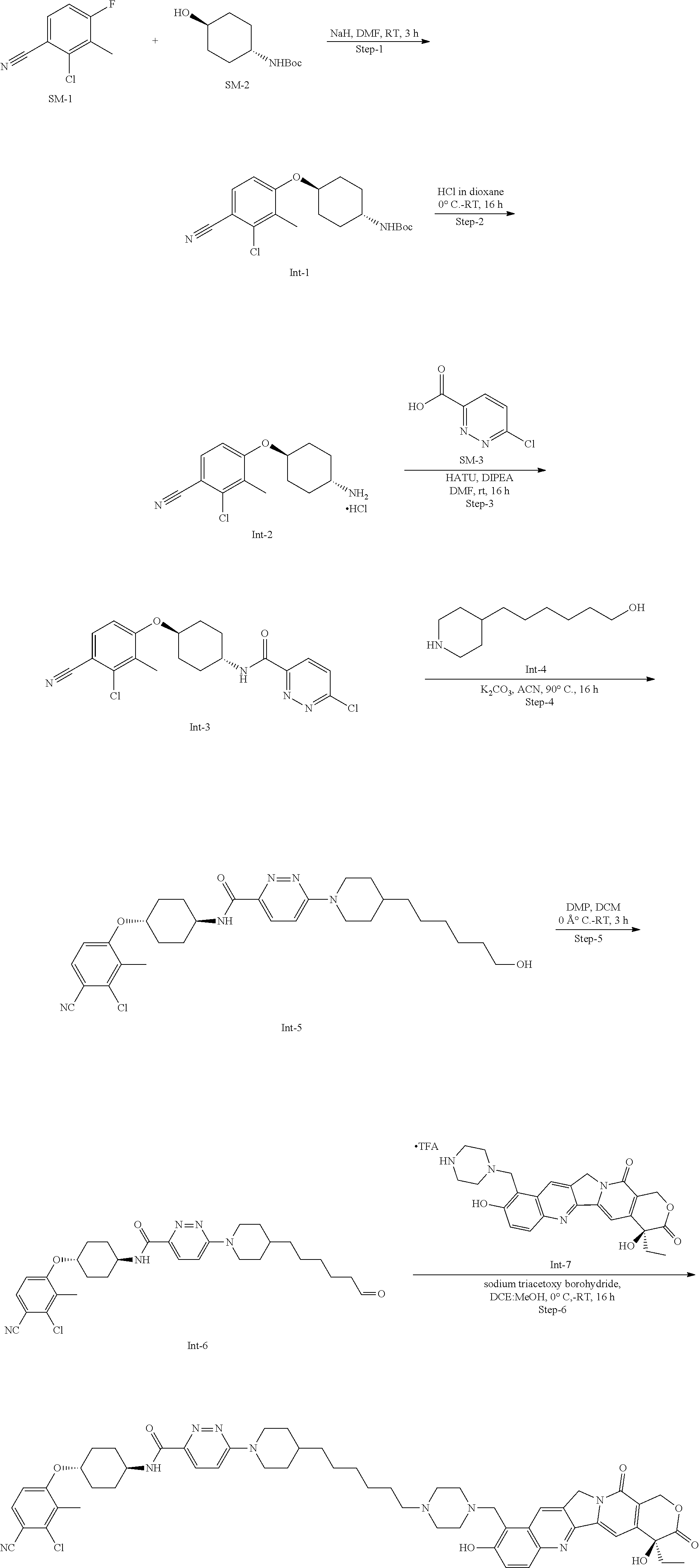

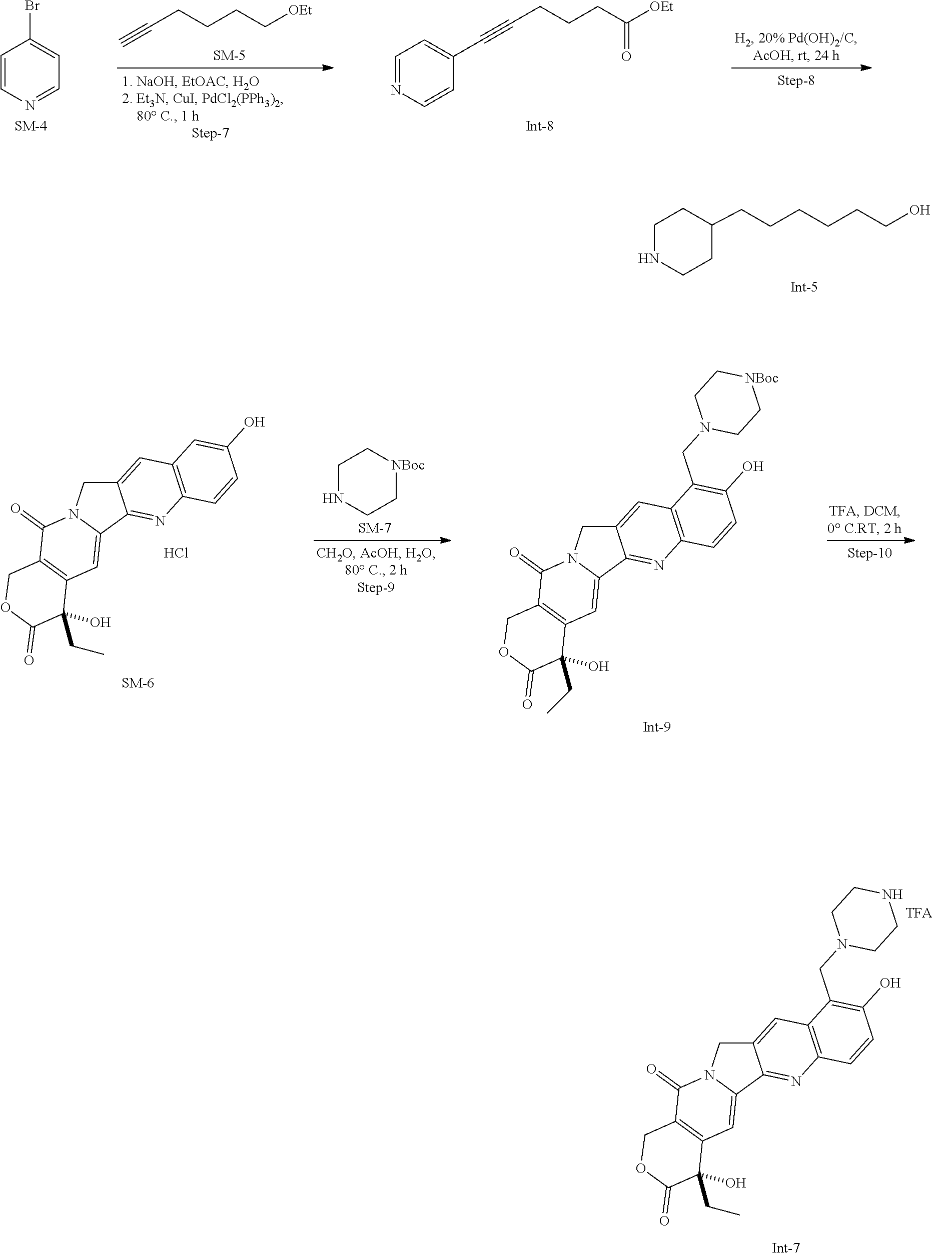

### Step-1: Synthesis of tert-butyl ((1r,4r)-4-(3-chloro-4-cyano-2-methylphenoxy)cyclohexyl) carbamate (Int-1)

To a stirred solution of tert-butyl ((1r,4r)-4-hydroxycyclohexyl)carbamate, (SM-2, 10 g, 46.5 mmol) in DMF (100 mL) under nitrogen atmosphere was added NaH (4.08 g, 102 mmol) at 0° C. and warmed to room temperature and stirred for 30 min. And then 2-chloro-4-fluoro-3-methylbenzonitrile (SM-1, 7.86 g, 46.51 mmol) was added portion wise over a period of 10 min at room temperature and stirred for 3 h. Progress of the reaction was monitored by TLC. After complete consumption of starting materials, the reaction mixture was poured into ice cold water (300 mL) and the precipitated solids were filtered and dried under vacuum to afford tert-butyl ((1r,4r)-4-(3-chloro-4-cyano-2-methyl phenoxy)cyclohexyl)carbamate (Int-1, 15 g, 88%) as an off-white solid.

### Step-2: Synthesis of 4-(((1r,4r)-4-aminocyclohexyl)oxy)-2-chloro-3-methylbenzonitrile hydrochloride (Int-2)

To a stirred solution of tert-butyl ((1r,4r)-4-(3-chloro-4-cyano-2-methylphenoxy) cyclohexyl)carbamate (Int-1, 15 g, 41 mmol) was added 4M HCl in 1,4-dioxane (75 mL) at 0° C. and stirred for 16 h at room temperature. Progress of the reaction was monitored by TLC. After complete consumption of starting materials, the volatiles were evaporated under reduced pressure and washed with diethyl ether, dried under vacuum to afford 4-(((1r,4r)-4-aminocyclohexyl)oxy)-2-chloro-3-methylbenzonitrile hydrochloride (Int-2, 15 g, crude) as an off-white solid.

### Step-3: Synthesis of 6-chloro-N-((1r,4r)-4-(3-chloro-4-cyano-2-methylphenoxy) cyclohexyl)pyridazine-3-carboxamide (Int-3)

To the mixture of 4-(((1r,4r)-4-aminocyclohexyl)oxy)-2-chloro-3-methyl benzonitrile hydrochloride (Int-2, 15 g, 56.9 mmol) and 6-chloropyridazine-3-carboxylic acid (SM-3, 9 g, 56.9 mmol) in DMF (100 mL) were added HATU (32.4 g, 85.44 mmol) and DIPEA (49 mL, 284 mmol) at 0° C. and the resultant reaction mixture was stirred at room temperature for 16 h. Progress of the reaction was monitored by TLC. After complete consumption of starting materials, volatiles were evaporated under reduced pressure and the obtained crude material was diluted with water (500 mL) and extracted with ethyl acetate (3×300 mL). The combined organic extracts were washed with water (400 mL) and brine (400 mL), dried over anhydrous sodium sulphate, filtered, and concentrated under reduced pressure to afford crude compound, which was purified by column chromatography by eluting with 60-80% ethyl acetate in n-heptane to afford 6-chloro-N-((1r,4r)-4-(3-chloro-4-cyano-2-methylphenoxy)cyclohexyl) pyridazine-3-carboxamide (Int-3, 6 g, 27%) as an off-white solid.

### Step-4: Synthesis of N-((1r,4r)-4-(3-chloro-4-cyano-2-methylphenoxy)cyclohexyl)-6-(4-(6-hydroxyhexyl)piperidin-1-yl)pyridazine-3-carboxamide (Int-5)

To a stirred solution of 6-chloro-N-((1r,4r)-4-(3-chloro-4-cyano-2-methylphenoxy)cyclohexyl)pyridazine-3-carboxamide (Int-3, 250 mg, 0.62 mmol) and 6-(piperidin-4-yl)hexan-1-ol (Int-4, 228 mg, 1.234 mmol) in acetonitrile (5 mL) was added potassium carbonate (603 mg, 1.85 mmol) at room temperature and the resultant reaction mixture was heated to 90° C. and stirred for 16 h. Progress of the reaction was monitored by TLC/LCMS. After complete consumption of starting materials, the reaction mixture was diluted with water (50 mL) and extracted with EtOAc (2×30 mL). The combined organic extracts were washed with water (30 mL) and brine (15 mL), dried over anhydrous sodium sulphate, filtered, and concentrated under reduced pressure to afford crude product, which was purified by combi-flash column chromatography by eluting with 60-65% ethyl acetate in n-heptane to afford N-((1r,4r)-4-(3-chloro-4-cyano-2-methylphenoxy)cyclohexyl)-6-(4-(6-hydroxyhexyl)piperidin-1-yl)pyridazine-3-carboxamide (Int-5, 200 mg, 58%) as an off-white solid.

### Step-5: Synthesis of N-((1r,4r)-4-(3-chloro-4-cyano-2-methylphenoxy)cyclohexyl)-6-(4-(6-oxohexyl)piperidin-1-yl)pyridazine-3-carboxamide (Int-6)

A stirred solution of N-((1r,4r)-4-(3-chloro-4-cyano-2-methylphenoxy)cyclohexyl)-6-(4-(6-hydroxyhexyl)piperidin-1-yl)pyridazine-3-carboxamide (Int-5, 200 mg, 0.36 mmol) in DCM (10 mL), Dess-Martin periodinane (DMP) (306 mg, 0.723 mmol) was added portion wise at 0° C. and the resultant reaction mixture was stirred at ambient temperature for 3 h. Progress of the reaction was monitored by TLC. After complete consumption of starting material, the reaction mixture was quenched with 50% aqueous Na₂S₂O₃ solution (10 mL), sat. NaHCO₃ solution (30 mL) and extracted with DCM (2×30 mL). The combined organic extracts were washed with brine (30 mL) and dried over anhydrous sodium sulphate, filtered and concentrated under reduced pressure to afford N-((1r,4r)-4-(3-chloro-4-cyano-2-methylphenoxy)cyclohexyl)-6-(4-(6-oxohexyl)piperidin-1-yl)pyridazine-3-carboxamide (Int-6, 180 mg, 90%) as an off-white solid.

### Step-6: Synthesis of N-((1r,4r)-4-(3-chloro-4-isocyano-2-methylphenoxy)cyclohexyl)-6-(4-(6-(4-(((S)-4-ethyl-4,9-dihydroxy-3,14-dioxo-3,4,12,14-tetrahydro-1H-pyrano [3′,4′:6,7]indolizino[1,2-b]quinolin-10-yl)methyl)piperazin-1-yl)hexyl)piperidin-1-yl)pyridazine-3-carboxamide

To a stirred solution of N-((1r,4r)-4-(3-chloro-4-isocyano-2-methylphenoxy)cyclohexyl)-6-(4-(6-oxohexyl)piperidin-1-yl)pyridazine-3-carboxamide (Int-6, 200 mg, 0.25 mmol) in DCE (15 mL) and MeOH (5 mL) were added(S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFA (Int-7, 200 mg, 0.25 mmol) and Et₃N (0.25 mL, 1.7 mmol) at 0° C. The resultant reaction mixture was stirred at room temperature for 2 h. After that sodium triacetoxy borohydride (152 mg, 0.71 mmol) and acetic acid (1 mL) were added at 0° C. The resultant reaction mixture was stirred at room temperature for 16 h Progress of the reaction was monitored by TLC. After complete consumption of starting materials, the reaction mass was cooled to 0° C. and quenched with sat. NaHCO₃ solution (10 mL), extracted with 10% MeOH in DCM (2×50 mL). The combined organic layer was washed with water (20 mL) and brine (20 mL) dried over anhydrous Na₂SO₄ and concentrated under reduced pressure to obtain crude product, which was purified by reverse phase purification (by using 0.1% FA in water eluted in 55% Acetonitrile in 0.1% FA in water) to afford the title compound (15.5 mg, 6.4%) as an off white solid.

¹H NMR (400 MHz, DMSO-d₆): δ ppm 8.71-8.78 (m, 1H), 8.56 (d, J=8.13 Hz, 1H), 8.22 (s, 1H), 7.97 (d, J=9.26 Hz, 1H), 7.78 (dd, J=9.07, 7.44 Hz, 2H), 7.41 (d, J=9.13 Hz, 1H), 7.32 (d, J=9.63 Hz, 1H), 7.26 (t, J=4.38 Hz, 2H), 6.43-6.53 (m, 1H), 5.41 (s, 2H), 5.22-5.28 (m, 2H), 4.42-4.55 (m, 3H), 4.10 (s, 2H), 3.81-3.94 (m, 2H), 2.88-3.01 (m, 4H), 2.59 (d, J=1.50 Hz, 2H), 2.23-2.29 (m, 6H), 2.07-2.15 (m, 2H), 1.82-1.95 (m, 5H), 1.75 (d, J=11.76 Hz, 2H), 1.51-1.67 (m, 5H), 1.41 (d, J=5.50 Hz, 2H), 1.20-1.32 (m, 8H), 1.03-1.13 (m, 3H), 0.88 (t, J=7.38 Hz, 3H); ESI MS m/z calcd. For C₅₅H₆₄ClN₉O₇ ([M+H]⁺) 998.4; found 998.4.

### Step-7: Synthesis of 6-(pyridin-4-yl)hex-5-yn-1-ol (Int-8)

A solution of 4-bromopyridine. HCl salt. (SM-4, 5.0 g, 25.77 mmol) in EtOAc (250 mL) was cooled to 0° C. followed by addition of aqueous sodium hydroxide solution (10.3 g, 257.7 mmol) in water (100 mL) and then the reaction mixture was allowed to stir at room temperature for 30 minutes. After consumption of (SM-4) as monitored by TLC, both the layers were separated, and organic layer was washed with brine (100 mL) solution, dried over anhydrous Na₂SO₄, filtered, and concentrated under reduced pressure to give 4-bromopyridine (3.50 g, 86%, crude) (HCl salt free) as colourless liquid.

To a stirred solution of above prepared 4-bromopyridine (3.5 g, 22.29 mmol) and hex-5-yn-1-ol (SM-5, 2.18 g, 22.29 mmol) in triethyl amine (10 mL) was de gassed with Argon and added copper (I) iodide (84.7 mg, 0.44 mmol) and PdCl₂(PPh₃)₂ (156 mg, 0.22 mmol) at room temperature and the resultant reaction mixture was heated to 80° C. and stirred for 1 h. Progress of the reaction was monitored by TLC and LCMS. After complete consumption of starting materials, the reaction mixture was diluted with water (150 mL) and extracted with EtOAc (2×80 mL). The combined organic layers were washed with water (75 mL), brine (80 mL) and dried over anhydrous Na₂SO₄, filtered and concentrated under reduced pressure to give crude product, which was purified by combi flash column chromatography by eluting with 70% EtOAc in heptane to afford 6-(pyridin-4-yl)hex-5-yn-1-ol (Int-8, 3.8 g, 97%) as a light-yellow oil.

### Step-8: Synthesis of 6-(piperidin-4-yl)hexan-1-ol (Int-5)

To a solution of 6-(pyridin-4-yl)hex-5-yn-1-ol (Int-8, 3.8 g, 21.7 mmol) and acetic acid (80 mL) in a autoclave was added 20% Pd(OH)₂/C (1.60 g) and filled with H₂ gas up to 10 kg and the resultant reaction mixture was allowed to stir at ambient temperature for 24 h. Progress of the reaction was monitored by TLC. After complete consumption of starting material, the reaction mixture was filtered through a celite bed and washed with EtOAc (100 mL). The combined filtrate was concentrated under reduced pressure to afford 6-(piperidin-4-yl)hexan-1-ol (Int-5, 3.5 g, 87%) as a brown oil, which was used as such in next step without any further purification.

### Step-9: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-9)

To a stirred solution of (S)-4-ethyl-4,9-dihydroxy-1,12-dihydro-14H-pyrano[3′,4′:6,7]indolizino[1,2-b]quinoline-3,14(4H)-dione·HCl salt (SM-6, 2.5 g, 6.86 mmol, 1.0 eq) and tert-butyl piperazine-1-carboxylate (SM-7, 1.85 g, 10.3 mmol, 1.5 eq) in acetic acid (10 mL) under inert atmosphere was added 37% solution of formaldehyde (0.29 mL, 8.24 mmol, 1.2 eq) at room temperature. The resultant reaction mixture was heated to 80° C. and stirred for 2 h in a sealed tube. Progress of the reaction was monitored by TLC. After complete consumption of starting materials, the volatiles were evaporated under reduced pressure to obtain crude compound, which was basified with aq. ammonia until the pH reached to 9, filtered the precipitated solid and washed with water (10 mL) and dried under vacuum to afford tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-9, 1.9 g,

### Step-10: Synthesis of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFAsalt (Int-7)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1carboxylate (Int-9, 2 g, 3.5 mmol, 1.0 eq) in DCM (20 mL) under nitrogen atmosphere was added TFA (2.7 mL, 35 mmol, 10 eq) at 0° C. The resultant reaction mixture was warmed to room temperature and stirred for 2 h. Progress of the reaction was monitored by TLC. After complete consumption of starting materials, the volatiles were evaporated under reduced pressure to obtain crude compound, which was washed with diethyl ether (10 mL) and dried under vacuum to afford(S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione (Int-7, 1.4 g, 83%) as a yellow solid.

### Example S53. Preparation of (S)-2-chloro-4-(3-(4-(4-((4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)methyl)piperidin-1-yl)phenyl)-4,4-dimethyl-5-oxo-2-thioxoimidazolidin-1-yl)benzonitrile (Compound No. 53)

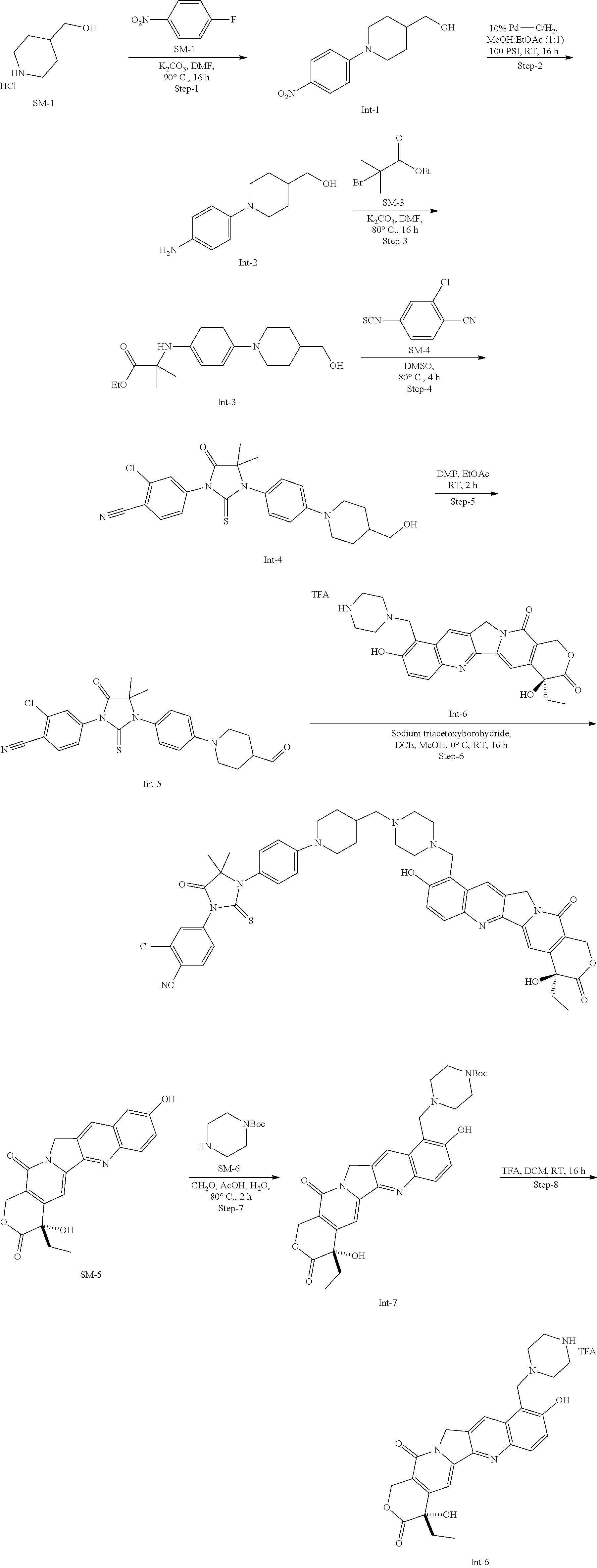

### Step-1: Synthesis of (1-(4-nitrophenyl)piperidin-4-yl)methanol (Int-1)

To a stirred solution of piperidin-4-ylmethanol·HCl (SM-1, 30 g, 197.8 mmol, 1.0 eq) in DMF (600 mL) were added 1-fluoro-4-nitrobenzene (SM-2, 33.4 g, 237.4 mmol, 1.2 eq) and K₂CO₃ (81.9 g, 593.5 mmol, 3.0 eq) at 0° C., under argon atmosphere. The resulting reaction mixture was stirred at 90° C. for 16 h. Progress of the reaction was monitored by TLC. After complete consumption of starting materials, the reaction mixture was diluted with water (1 L) and filtered the obtained solids, washed with water (500 mL) and dried under vacuum to afford (1-(4-nitrophenyl)piperidin-4-yl)methanol (Int-1, 40 g, 87%) as a pale yellow solid.

### Step-2: Synthesis of (1-(4-aminophenyl)piperidin-4-yl)methanol (Int-2)

To a stirred solution of (1-(4-nitrophenyl)piperidin-4-yl)methanol (Int-1, 10 g, 42.3 mmol, 1.0 eq) in McOH:EtOAc (1:1, 300 mL) was added 10% Pd—C (2 g, 20% w/w) at ambient temperature, under argon atmosphere. The resultant reaction mixture was stirred under Hydrogen atmosphere (100 PSI) for 16 h (Autoclave). Progress of the reaction was monitored by TLC. After complete consumption of starting materials, the reaction mixture was filtered through celite bed and washed with MeOH (200 mL), the obtained filtrate was evaporated under reduced pressure to afford crude (1-(4-aminophenyl)piperidin-4-yl)methanol (Int-2, 10 g) as a pale yellow gummy liquid, Note: Proceeded next step without purification.

### Step-3: Synthesis of ethyl 2-((4-(4-(hydroxymethyl)piperidin-1-yl)phenyl)amino)-2-methylpropanoate (Int-3)

To a stirred solution of (1-(4-aminophenyl)piperidin-4-yl)methanol (Int-2, 10 g, 48.54 mmol, 1.0 eq) in DMF (200 mL) were added ethyl 2-bromo-2-methylpropanoate (SM-3, 11.35 g, 58.25 mmol, 1.2 eq) and K₂CO₃ (20.09 g, 145.63 mmol, 3.0 eq) at 0° C., under argon atmosphere. The resulting reaction mixture was stirred at 80° C. for 16 h. Progress of the reaction was monitored by TLC. After complete consumption of starting materials, the reaction mixture was diluted with water (500 mL) and extracted with ethyl acetate (2×500 mL). The combined organic layer was washed with water (200 mL), brine (200 mL) and dried over anhydrous sodium sulphate, filtered and concentrated under reduced pressure to obtain crude compound, which was purified by flash column chromatography to afford ethyl 2-((4-(4-(hydroxymethyl)piperidin-1-yl)phenyl)amino)-2-methylpropanoate (Int-3, 10 g, 64%) as a pale yellow solid.

### Step-4: Synthesis of 2-chloro-4-(3-(4-(4-(hydroxymethyl)piperidin-1-yl)phenyl)-4,4-dimethyl-5-oxo-2-thioxoimidazolidin-1-yl)benzonitrile (Int-4)

To a stirred solution of ethyl 2-((4-(4-(hydroxymethyl)piperidin-1-yl)phenyl)amino)-2-methylpropanoate (Int-3, 30 g, 93.75 mmol, 1.0 eq) in DMSO (120 mL) was added 2-chloro-4-isothiocyanatobenzonitrile (SM-4, 21.8 g, 112.5 mmol, 1.2 eq) at 0° C., under argon atmosphere. The resultant reaction mixture was stirred at 80° C. for 4 h. Progress of the reaction was monitored by TLC. After complete consumption of starting materials, the reaction mixture was diluted with water (500 mL) and filtered the obtained solids, washed with water (500 mL) and diethyl ether (300 mL), dried under vacuum to afford 2-chloro-4-(3-(4-(4-(hydroxymethyl)piperidin-1-yl)phenyl)-4,4-dimethyl-5-oxo-2-thioxoimidazolidin-1-yl)benzonitrile (Int-4, 35 g, 81%) as a pale yellow solid.

### Step-5: Synthesis of 2-chloro-4-(3-(4-(4-formylpiperidin-1-yl)phenyl)-4,4-dimethyl-5-oxo-2-thioxoimidazolidin-1-yl)benzonitrile (Int-5)

To a stirred solution of 2-chloro-4-(3-(4-(4-(hydroxymethyl)piperidin-1-yl)phenyl)-4,4-dimethyl-5-oxo-2-thioxoimidazolidin-1-yl)benzonitrile (Int-4, 1 g, 2.13 mmol) in EtOAc (20 mL) was added (portion wise) Dess-Martin periodinane (DMP, 1.35 mg, 3.19 mmol) at 0° C. and the resultant reaction mixture was stirred at ambient temperature for 2 h. Progress of the reaction was monitored by TLC. After complete consumption of starting materials, the reaction mixture was quenched with 50% aqueous Na₂S₂O₃ solution (50 mL), sat. NaHCO₃ solution (50 mL) and extracted with EtOAc (2×50 mL). The combined organic extracts were washed with brine (50 mL) and dried over anhydrous sodium sulphate, filtered and concentrated under reduced pressure to afford 2-chloro-4-(3-(4-(4-formylpiperidin-1-yl)phenyl)-4,4-dimethyl-5-oxo-2-thioxoimidazolidin-1-yl)benzonitrile (Int-5, 1 g, Crude) as a brown gummy liquid.

### Step-6: Synthesis of (S)-2-chloro-4-(3-(4-(4-((4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)methyl) piperidin-1-yl)phenyl)-4,4-dimethyl-5-oxo-2-thioxoimidazolidin-1-yl)benzonitrile

To a stirred solution of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFA (Int-6, 1 g, 2.14 mmol) in DCE (16 mL) and MeOH (4 mL) were added 2-chloro-4-(3-(4-(4-formylpiperidin-1-yl)phenyl)-4,4-dimethyl-5-oxo-2-thioxoimidazolidin-1-yl)benzonitrile (Int-5, 1 g, 0.21 mmol) and AcOH (0.5 mL, 9 mmol) at 0° C. The resultant mixture was stirred at room temperature for 1 h. After that sodium triacetoxy borohydride (907 mg, 4.28 mmol) was added at 0° C. The resultant reaction mixture was stirred at room temperature for 16 h. Progress of the reaction was monitored by TLC. After complete consumption of starting materials, the reaction mass was cooled to 0° C. and quenched with sat. NaHCO₃ solution (10 mL), extracted with 10% MeOH in DCM (2×50 mL), The combined organic layer was washed with water (20 mL) and brine (20 mL), dried over anhydrous Na₂SO₄, filtered and concentrated under reduced pressure to obtain crude product, which was purified by reverse phase prep HPLC. (using 0.1% FA in water eluted in 55% Acetonitrile in 0.1% FA in water) to afford the title compound (44 mg, 2.2%) as an off white solid.

¹H NMR (400 MHz, DMSO-d₆): δ ppm 8.74 (s, 1H), 8.15-8.21 (m, 2H), 7.96-8.02 (m, 2H), 7.69-7.73 (m, 1H), 7.40-7.44 (m, 1H), 7.26 (s, 1H), 7.12-7.15 (m, 2H), 7.03 (d, J=9.13 Hz, 2H), 6.48 (s, 1H), 5.41 (s, 2H), 5.25 (s, 2H), 4.11 (s, 2H), 3.78 (d, J=12.13 Hz, 2H), 2.71-2.77 (m, 4H), 2.59-2.63 (m, 2H), 2.19 (d, J=7.00 Hz, 2H), 1.82-1.91 (m, 4H), 1.65-1.73 (m, 2H), 1.46 (s, 6H), 1.17-1.25 (m, 4H), 0.88 (t, J=7.25 Hz, 3H); ESI MS m/z calcd. For C₄₉H₄₉ClN₈O₆S ([M+H]+) 913.32; found 913.50.

### Step-7: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-7)

To a stirred solution of (S)-4-ethyl-4,9-dihydroxy-1,12-dihydro-14H-pyrano[3′,4′:6,7]indolizino[1,2-b]quinoline-3,14(4H)-dione·HCl salt (SM-5, 2.5 g, 6.86 mmol, 1.0 eq) and tert-butyl piperazine-1-carboxylate (SM-6, 1.85 g, 10.3 mmol, 1.5 eq) in acetic acid (10 mL) under inert atmosphere was added 37% solution of formaldehyde (0.29 mL, 8.24 mmol, 1.2 eq) at room temperature. The resultant reaction mixture was heated to 80° C. and stirred for 2 h in a sealed tube. Progress of the reaction was monitored by TLC. After complete consumption of starting materials, the volatiles were evaporated under reduced pressure to obtain crude compound, which was basified with aq. ammonia until the pH reached to 9, filtered the precipitated solid and washed with water (10 mL) and dried under vacuum to afford tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-7, 1.9 g, 50%) as a yellow solid.

### Step-8: Synthesis of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFAsalt (Int-6)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1 carboxylate (Int-7, 2 g, 3.5 mmol, 1.0 eq) in DCM (20 mL) under nitrogen atmosphere was added TFA (2.7 mL, 35 mmol, 10 eq) at 0° C. The resultant reaction mixture was warmed to room temperature and stirred for 2 h. Progress of the reaction was monitored by TLC. After complete consumption of starting materials, the volatiles were evaporated under reduced pressure to obtain crude compound, which was washed with diethyl ether (10 mL) and dried under vacuum to afford(S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione (Int-6, 1.4 g, 83%) as a yellow solid.

### Example S54. Preparation of N-((1r,4r)-4-(3-chloro-4-isocyano-2-methylphenoxy)cyclohexyl)-6-(4-(6-(4-(((S)-4-ethyl-4,9-dihydroxy-3,14-dioxo-3,4,12,14-tetrahydro-1H-pyrano [3′,4′:6,7]indolizino[1,2-b]quinolin-10-yl)methyl)piperazin-1-yl)hexyl)piperidin-1-yl)pyridazine-3-carboxamide (Compound No. 54)

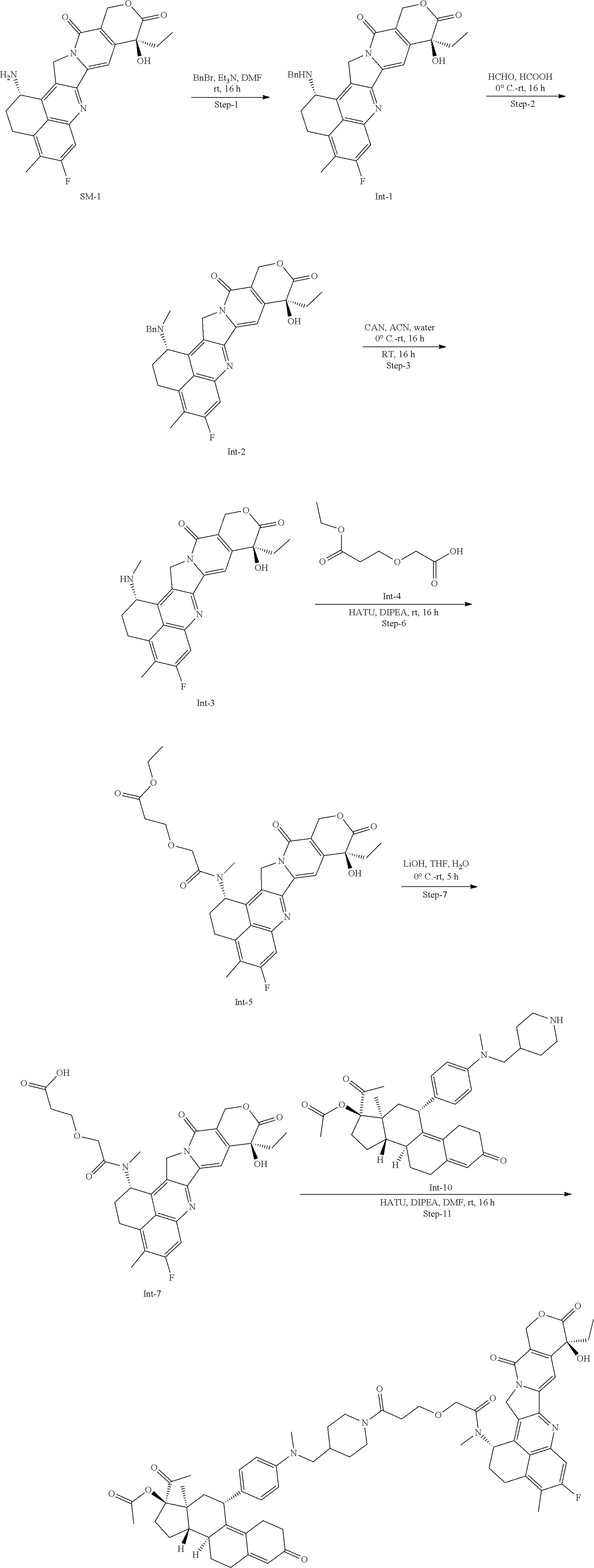

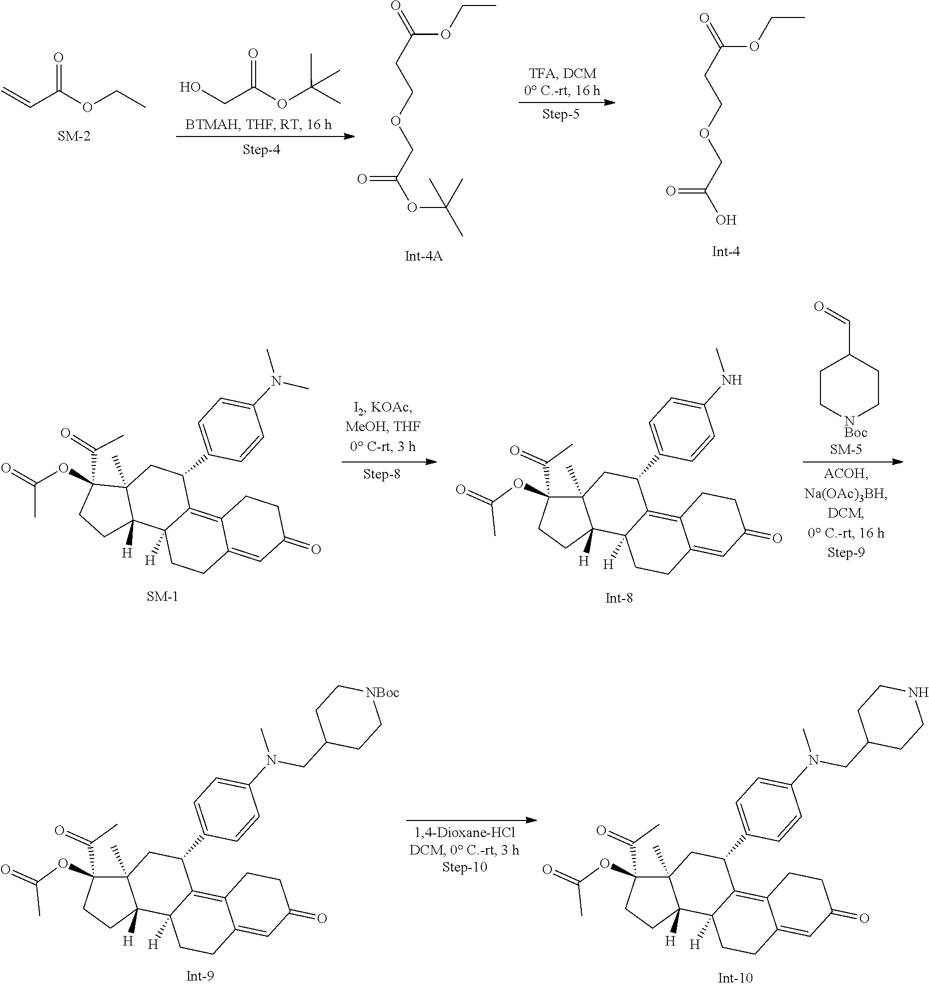

### Step-1: Synthesis of (1S,9S)-1-(benzylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-1)

To a solution of ((1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (SM-1, 5 g, 9.40 mmol, 1.0 eq) in DMF (50 mL), DIPEA (4.9 mL, 28.20 mmol, 3.0 eq) was added at room temperature and stirred for 5 min. Benzyl bromide (1.6 g, 9.40 mmol, 1.0 eq) was added drop-wise and the resultant reaction mixture was stirred at room temperature for 16 h. Progress of the reaction was monitored by TLC. After complete consumption of starting materials, the reaction mixture was poured into cold water (300 mL) and extracted with ethyl acetate (2×200 mL). The combined organic extracts were washed with brine (200 mL) and dried over anhydrous sodium sulphate, filtered and concentrated under vacuum to obtain crude compound, which was purified by combi-flash chromatography by eluting with 0-70% ethyl acetate in heptane to afford (1S,9S)-1-(benzylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-1, 4.2 g, 85%) as a pale yellow solid.

### Step-2: Synthesis of (1S,9S)-1-(benzyl(methyl)amino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-2)

To a solution of (1S,9S)-1-(benzylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-1, 3 g, 5.70 mmol, 1.0 eq) in DCM (50 mL). Formaldehyde (22.3 mL, 28.54 mmol, 5.0 eq) and Formic acid (0.10 ml, 2.84 mmol, 0.5 eq) were added at 0° C. and the resultant reaction mixture was stirred at room temperature for 16 h. Progress of the reaction was monitored by TLC. After complete consumption of starting material, the reaction mixture was poured into ice cold water (200 mL) and extracted with DCM (2×100 mL). The combined organic extracts were washed with brine (100 mL) and dried over anhydrous sodium sulphate, filtered and concentrated under reduced pressure to obtain crude compound, which was purified by combi-flash chromatography by eluting with 0-70% ethyl acetate in heptane to afford (1S,9S)-1-(benzyl (methyl)amino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-2, 2 g, 64%) as a pale brown solid.

### Step-3: Synthesis of (1S,9S)-9-ethyl-5-fluoro-9-hydroxy-4-methyl-1-(methylamino)-1,2,3,9,12,15-hexahydro-10H,13H-benzo[de]pyrano[3′,4′:6,7]indolizino[1,2-b]quinoline-10,13-dione (Int-3)

To a solution of (1S,9S)-1-(benzyl (methyl)amino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-2, 1.5 g, 2.80 mmol, 1.0 eq) in Acetonitrile (40 mL), CERIC AMMONIUM NITRATE (3 g, 5.60 mmol, 2.0 eq) in water (10 ml) was added at 0° C. and the resultant reaction mixture was stirred at room temperature for 16 h. Progress of the reaction was monitored by TLC. After complete consumption of the starting material, the reaction mixture was diluted with water (200 mL) and extracted with DCM (2×100 mL). The combined organic extracts were washed with brine (100 mL) and dried over anhydrous sodium sulphate, filtered and concentrated under reduced pressure to afford (1S,9S)-9-ethyl-5-fluoro-9-hydroxy-4-methyl-1-(methylamino)-1,2,3,9,12,15-hexahydro-10H,13H-benzo[de]pyrano[3′,4′:6,7]indolizino[1,2-b]quinoline-10,13-dione (Int-3, 1 g, 80%) as a pale brown solid.

### Step-4: Synthesis of ethyl 3-(2-(tert-butoxy)-2-oxoethoxy)propanoate (Int-4A)

To a solution of ethyl acrylate (SM-2, 5 g, 49.94 mmol, 1.0 eq) in THF (50 mL), were added Benzyltrimethylammonium Hydroxide (BTMAH, 1.67 g, 9.98 mmol, 0.2 eq) and tert-butyl 2-hydroxyacetate (SM-3, 6.6 g, 49.94 mmol, 1.0 eq) at 0° C. and the resultant reaction mixture was stirred at room temperature for 16 h. Progress of the reaction was monitored by TLC. After complete consumption of the starting material, the reaction mixture was poured into ice cold water (200 mL) and extracted with DCM (2×100 mL). The combined organic extracts were washed with brine (100 mL) and dried over anhydrous sodium sulphate, filtered and concentrated under reduced pressure to obtain crude compound, which was purified by combi-flash chromatography by eluting with 0-30% ethyl acetate in heptane to afford ethyl 3-(2-(tert-butoxy)-2-oxoethoxy)propanoate (Int-4A, 4 g, 34%) as a pale yellow oil.

### Step-5: Synthesis of 2-(3-ethoxy-3-oxopropoxy)acetic acid (Int-4)

To a stirred solution of ethyl 3-(2-(tert-butoxy)-2-oxoethoxy)propanoate (Int-4A, 2 g, 8.61 mmol, 1 eq) in dichloromethane (20 mL) was added trifluoro acetic acid (2 mL) at 0° C. and then allowed to stir at room temperature for 16 h. Progress of the reaction was monitored by TLC. After complete consumption of starting material, the reaction mixture was evaporated under reduced pressure, washed with diethyl ether and dried under vacuum to afford 2-(3-ethoxy-3-oxopropoxy)acetic acid (Int-4, 1.3 g, 86%) as a colourless oil.

### Step-6: Synthesis of ethyl 3-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)(methyl)amino)-2-oxoethoxy)propanoate (Int-5)

To a stirred solution of (1S,9S)-9-ethyl-5-fluoro-9-hydroxy-4-methyl-1-(methylamino)-1,2,3,9,12,15-hexahydro-10H,13H-benzo[de]pyrano[3′,4′:6,7]indolizino[1,2-b]quinoline-10,13-dione (Int-3, 3.7 g, 8.20 mmol, 1.0 eq) in DMF (50 mL), 2-(3-ethoxy-3-oxopropoxy)acetic acid (Int-4, 1.45 g, 8.20 mmol, 1 eq) and N,N-Diisopropylethylamine (4.3 ml, 25.0 mmol, 3.0 eq) were added at room temperature and stirred for 5 min. After that HATU (4.8 g, 12.0 mmol, 1.5 eq) was added at room temperature. The resultant reaction mixture was stirred at room temperature for 16 h. Progress of the reaction was monitored by TLC. After complete consumption of starting material, the reaction mixture was diluted with ice cold water (300 mL) and extracted with EtOAc (2×200 mL). The combined organic layer was washed with brine solution (100 mL), dried over anhydrous sodium sulphate, filtered, and concentrated under reduced pressure to obtain crude compound, which was purified by combiflash chromatography by eluting with 0-5% MeOH in DCM to afford ethyl 3-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)(methyl)amino)-2-oxoethoxy)propanoate (Int-5, 1.6 g, 32%) as a pale brown solid.

### Step-7: Synthesis of 3-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)(methyl)amino)-2-oxoethoxy)propanoic acid (Int-6)

To a stirred solution of ethyl 3-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)(methyl)amino)-2-oxoethoxy)propanoate (Int-5, 800 mg, 1.31 mmol, 1.0 eq) in THF (20 mL) was added LiOH (47 mg, 1.97 mmol, 1.5 eq) in water (10 ml) at 0° C. The resultant reaction mixture was stirred at room temperature for 5 h. Progress of the reaction was monitored by TLC. After complete consumption of starting material, the reaction mixture was diluted with ice cold water (100 mL), acidified with 1N HCl solution and extracted with DCM (2×100 mL). The combined organic layer was washed with brine solution (50 mL), dried over anhydrous sodium sulphate, filtered, and concentrated under reduced pressure to afford 3-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)(methyl)amino)-2-oxoethoxy)propanoic acid (Int-6, 400 mg, 52%) as a pale brown solid.

### Step-8: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-7)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-1, 10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The resultant reaction mixture was allowed to room temperature and stirred for 3 h. Progress of the reaction was monitored by TLC. After complete consumption of starting materials, the reaction mixture was quenched with sodium thiosulfate (Na₂S₂O₃) solution (50 g in 30 mL water) and extracted with ethyl acetate (2×200 mL), The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 8.0 g, 82%) as an off-white solid which was used in next step without any further purification.

### Step-9: Synthesis of tert-butyl 4-(((4-((8R,11S,13R,14R,17S)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)methyl)piperidine-1-carboxylate (Int-8)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-7, 1.2 g, 2.60 mmol, 1 eq) in DCM (20 mL), tert-butyl 4-formylpiperidine-1-carboxylate (SM-5, 610 mg, 2.90 mmol, 1.1 eq) and acetic acid (2 ml) at 0° C. The resultant reaction was stirred at room temperature for 3 h. After sodium triacetoxy borohydride (1.1 g, 5.20 mmol, 2.0 eq) was added to above reaction mixture at 0° C. The resultant reaction mixture was stirred at room temperature for 16 h. Progress of the reaction was monitored by TLC. After complete completion of starting materials, the reaction mass was cooled to 0° C. quenched with sat. NaHCO₃ solution (100 mL), extracted with in DCM (2×100 mL) combined organic layers were washed with water (50 mL) and brine (50 mL) dried over Na₂SO₄ and concentrated to obtained crude compound, which was purified by combiflash chromatography by eluting with 0-5% MeOH in DCM to afford tert-butyl 4-(((4-((8R,11S,13R,14R,17S)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)methyl)piperidine-1-carboxylate (Int-8, 1 g, 58%) as white solid.

### Step-10: Synthesis of (8S,11R,13S,14S,17R)-17-acetyl-13-methyl-11-(4-(methyl(piperidin-4-ylmethyl)amino)phenyl)-3-oxo-2,3,6,7,8,11,12,13,14,15,16,17-dodecahydro-1H-cyclopenta[a]phenanthren-17-yl acetate (Int-9)

To a stirred solution of tert-butyl 4-(((4-((8R,11S,13R,14R,17S)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)methyl)piperidine-1-carboxylate (Int-8, 1 g, 1.50 mmol, 1 eq) in dichloromethane (20 mL) was added 4M 1,4-Dioxane-HCl (5 mL) at 0° C. and then allowed to stir at room temperature for 3 h. Progress of the reaction was monitored by TLC. After complete consumption of starting material, the reaction mixture was evaporated under reduced pressure, washed with diethyl ether and dried under reduced pressure to afford (8S,11R,13S,14S,17R)-17-acetyl-13-methyl-11-(4-(methyl (piperidin-4-ylmethyl)amino)phenyl)-3-oxo-2,3,6,7,8,11,12,13,14,15,16,17-dodecahydro-1H-cyclopenta[a]phenanthren-17-yl acetate (Int-9, 800 mg, 94%) as white solid.

### Step-11: Synthesis of (8S,11R,13S,14S,17R)-17-acetyl-11-(4-(((1-(3-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)(methyl)amino)-2-oxoethoxy)propanoyl)piperidin-4-yl)methyl)(methyl)amino)phenyl)-13-methyl-3-oxo-2,3,6,7,8,11,12,13,14,15,16,17-dodecahydro-1H-cyclopenta[a]phenanthren-17-yl acetate

To a stirred solution of 3-(2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)(methyl)amino)-2-oxoethoxy)propanoic acid (Int-6, 500 mg, 0.86 mmol, 1.0 eq) in DMF (5 mL), (8S,11R,13S,14S,17R)-17-acetyl-13-methyl-11-(4-(methyl (piperidin-4-ylmethyl)amino)phenyl)-3-oxo-2,3,6,7,8,11,12,13,14,15,16,17-dodecahydro-1H-cyclopenta[a]phenanthren-17-yl acetate (Int-9, 530 g, 0.94 mmol, 1.1 eq) and N,N-Diisopropylethylamine (0.32 ml, 2.58 mmol, 3.0 eq) were added at room temperature and stirred for 5 min. After that HATU (507 mg, 1.29 mmol, 1.5 eq) was added at room temperature. The resultant reaction mixture was stirred at room temperature for 16 h. The reaction was monitored by TLC. After complete completion of starting material, the reaction mixture was dilute with ice cold water (100 mL) and extracted with EtOAc (2×100 mL). The combined organic layer was washed with brine solution (50 mL), dried over anhydrous sodium sulphate, filtered, and concentrated under reduced pressure to obtained crude compound, which was purified by combiflash chromatography by eluting with 0-5% MeOH in DCM to afford title compound (150 mg, 15%) as an off-white solid.

¹H NMR (400 MHz, DMSO-d₆): δ ppm 7.67-7.87 (m, 1H), 7.25-7.38 (m, 1H), 6.81-7.10 (m, 2H), 6.27-6.80 (m, 3H), 5.56-6.06 (m, 1H), 5.33-5.49 (m, 2H), 4.44-5.31 (m, 3H), 3.84-4.43 (m, 4H), 3.51-3.84 (m, 2H), 3.38-3.49 (m, 1H), 2.99-3.24 (m, 3H), 2.56-2.98 (m, 10H), 2.44-2.48 (m, 2H), 2.32-2.42 (m, 5H), 2.02-2.31 (m, 8H), 1.99 (br s, 5H), 1.66-1.88 (m, 5H), 1.33-1.64 (m, 4H), 1.17-1.32 (m, 2H), 1.07-1.16 (m, 1H), 0.93-1.06 (m, 1H), 0.68-0.88 (m, 4H), 0.39-0.65 (m, 1H), -0.13-0.33 (m, 3H); LCMS: 93.85%; ESI MS m/z calcd. For C₆₅H₇₄FN₅O₁₁ ([M−H]⁺) 1120.3; found 1119.9; HPLC: 95.03%.

### Example S55. Preparation of (8S,11R,13S,14S,17R)-17-acetyl-11-(4-((7-(4-((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)(methyl)amino)-2-oxoethoxy)methyl)piperidin-1-yl) heptyl)(methyl)amino)phenyl)-13-methyl-3-oxo-(Compound No. 55)

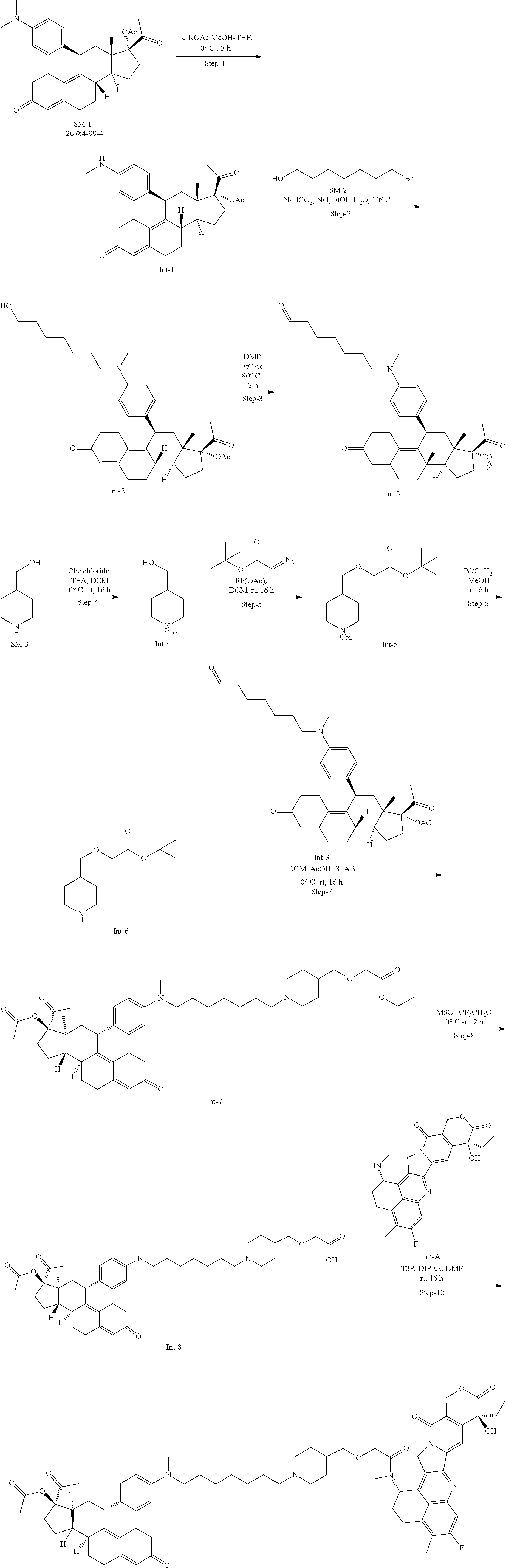

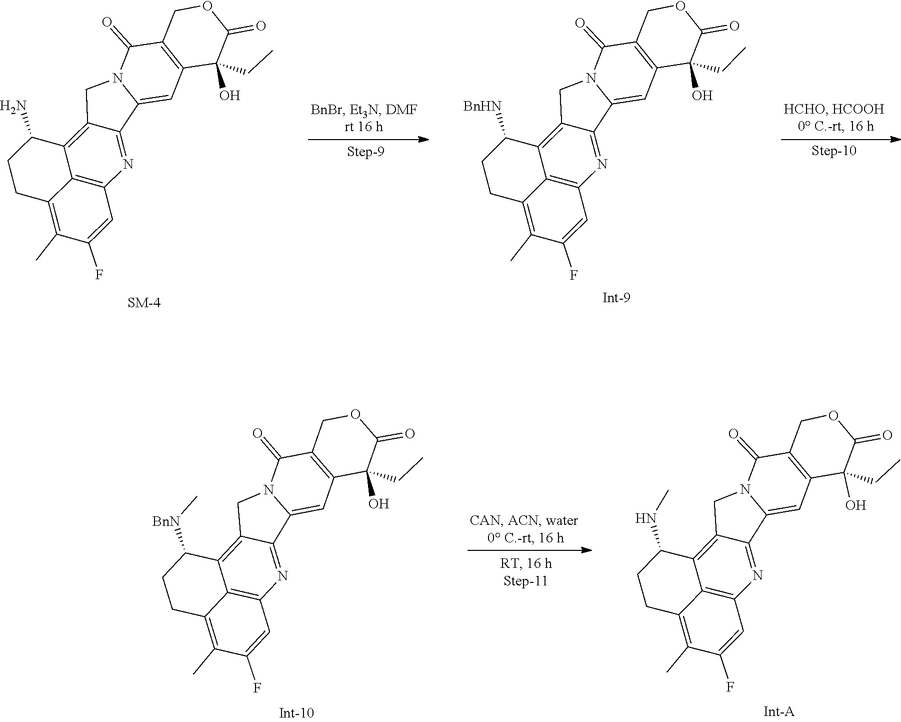

### Step-1: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-1, 10 g, 21 mmol, 1.0 eq) in methanol (150 mL) and THF (150 mL), KOAc (20.6 g, 210 mmol, 10 eq) and Iodine (13.1 g, 105 mmol, 5 eq) were added at 0° C. The resultant reaction mixture was allowed to room temperature and stirred for 3 h. Progress of the reaction was monitored by TLC. After complete consumption of the starting materials, the reaction mixture was quenched with sodium thiosulfate (Na₂S₂O₃) solution (50 g in 30 mL water) and extracted with ethyl acetate (2×200 mL), The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-2, 8.0 g, 82%) as an off-white solid, which was used in next step without any further purification.

### Step-2: Synthesis of (8S,11R,13S,14S,17R)-17-acetyl-11-(4-((7-hydroxyheptyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 5.0 g, 10.84 mmol, 1.0 eq) in EtOH (50 mL, 10 vol) and H₂O (25 mL, 5 vol). 7-bromoheptan-1-ol (SM-2, 10.56 g, 54.2 mmol, 5.0 eq) and NaHCO₃ (2.73 g, 32.52 mmol, 3.0 eq) were added at 0° C. under argon atmosphere. The resultant reaction mixture was stirred at 80° C. for 16 h. The reaction was monitored by TLC. After complete consumption of starting materials, the reaction mixture was diluted with ice cold water (250 mL) and extracted with EtOAc (2×500 mL), The combined organic layer was washed with brine solution (200 mL), dried over anhydrous sodium sulphate, filtered, and concentrated under reduced pressure to obtain crude compound, which was purified by flash column (silica gel. 100-200 mesh) elution with 20-50% EtOAc in Hexane to afford (8S,11R,13S,14S,17R)-17-acetyl-11-(4-((7-hydroxyheptyl)(methyl)amino)phenyl)-13-methyl-3-oxo-2,3,6,7,8,11,12,13,14,15,16, 17-dodecahydro-1H-cyclopenta[a]phenanthren-17-yl acetate (Int-2, 2 g, 32%) as an off white solid.

### Step-3: Synthesis of (8S,11R,13S,14S,17R)-17-acetyl-13-methyl-11-(4-(methyl(7-oxoheptyl)amino)phenyl)-3-oxo-2,3,6,7,8,11,12,13,14,15,16,17-dodecahydro-1H-cyclopenta[a]phenanthren-17-yl acetate (Int-3)

To a stirred solution of (8S,11R,13S,14S,17R)-17-acetyl-11-(4-((7-hydroxyheptyl) (methyl)amino)phenyl)-13-methyl-3-oxo-2,3,6,7,8,11,12,13,14,15,16,17-dodecahydro-1H-cyclopenta[a]phenanthren-17-yl acetate (Int-2, 3 g, 5.2 mmol, 1.0 eq) in EtOAc (60 mL, 20 vol). DMP (4.4 g, 10.4 mmol, 2.0 eq) was added at 0° C. under argon atmosphere. The resultant reaction mixture was stirred at 80° C. for 2 h. Progress of the reaction was monitored by TLC. After complete consumption of starting materials, the reaction mixture was quenched with mixture of Na₂S₂O₃ and saturated Sodium bicarbonate solution (1:1, 250 mL) and extracted with EtOAc (2×500 mL). The combined organic layer was washed with brine solution (200 mL), dried over anhydrous sodium sulphate, filtered and concentrated under reduced pressure to obtain crude (8S,11R,13S,14S,17R)-17-acetyl-13-methyl-11-(4-(methyl(7-oxoheptyl)amino)phenyl)-3-oxo-2,3,6,7,8,11,12,13,14,15,16,17-dodecahydro-1H-cyclopenta[a]phenanthren-17-yl acetate (Int-2, 3 g, crude) as an off white solid. The crude was taken to next step without any further purification.

### Step-4: Synthesis of benzyl 4-(hydroxymethyl)piperidine-1-carboxylate (Int-4)

To a stirred solution of 4-piperidylmethanol (SM-2, 5.0 g, 43 mmol) in DCM (50 mL). Triethyl amine (8.8 g, 87 mmol, 2.0 eq) and Benzyl chloroformate in TOLUENE (29.6 mL, 87 mmol, 2.0 eq) were added at 0° C. The resultant reaction mixture was stirred at room temperature for 16 h. The reaction was monitored by TLC. After complete consumption of starting material, the reaction mixture was quenched with water, extracted with EtOAc (3×100 mL). The combined organic layers were dried over anhydrous Na₂SO₄, filtered and concentrated to get crude compound, which was purified by flash column (silica gel. 100-200 mesh) eluted with 20-50% EtOAc in Hexane to afford Benzyl 4-(hydroxymethyl)piperidine-1-carboxylate (Int-4, 3.8 g, 35%) as a colourless liquid.

### Step-5: Synthesis of benzyl 4-((2-(tert-butoxy)-2-oxoethoxy)methyl)piperidine-1-carboxylate (Int-5)

To a stirred solution of Benzyl 4-(hydroxymethyl)piperidine-1-carboxylate (Int-4, 1.0 g, 4.03 mmol) in DCM (10 mL) were added tert-butyl 2-diazoacetate (1.1 g, 8.0 mmol, 2.0 eq) and RHODIUM (II) ACETATE DIMER (0.18 g, 0.40 mmol, 0.1 eq) at room temperature. The resultant reaction mixture was stirred at room temperature for 16 h. Progress of the reaction was monitored by TLC. After complete consumption of starting material, the reaction mixture was evaporated under reduced pressure to get crude compound, which was purified by flash column (silica gel. 100-200 mesh) elution with 20-30% EtOAc in Hexane to afford benzyl 4-((2-(tert-butoxy)-2-oxoethoxy)methyl)piperidine-1-carboxylate (Int-5, 600 mg, 41%) as a colourless liquid.

### Step-6: Synthesis of tert-butyl 2-(piperidin-4-ylmethoxy)acetate (Int-6)

To a stirred solution of benzyl 4-((2-(tert-butoxy)-2-oxoethoxy)methyl)piperidine-1-carboxylate (Int-5, 2.0 g, 5.5 mmol) in Methonal (50 mL) 10% pelladium on Carban (400) was added at room temperature. The resultant reaction mixture was stirred at room temperature for 6 h under Hydrogen gas atmosphere (100 PSI). Progress of the reaction was monitored by TLC. After complete consumption of starting material, the reaction mixture was filtered through a celite bed and washed with Methanol (100 mL). The filtrate was evaporated under reduced pressure to afford tert-butyl 2-(piperidin-4-ylmethoxy)acetate (Int-6, 1.2 g, 95%) as a colourless liquid.

### Step-7: Synthesis of tert-butyl 2-((1-(7-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)heptyl)piperidin-4-yl)methoxy)acetate (Int-7)

To a stirred solution of (8S,11R,13S,14S,17R)-17-acetyl-13-methyl-11-(4-(methyl(7-oxoheptyl)amino)phenyl)-3-oxo-2,3,6,7,8,11,12,13,14,15,16,17-dodecahydro-1H-cyclopenta[a]phenanthren-17-yl acetate (Int-3, 500 mg, 0.87 mmol, 1 eq) in DCE (10 mL), were added tert-butyl 2-(piperidin-4-ylmethoxy)acetate (200 mg, 0.87 mmol, 1 eq) and acetic acid (0.26 g, 4.35 mmol, 5 eq) at 0° C. The resultant reaction mixture was stirred at room temperature for 2 h. After that sodium triacetoxy borohydride (1.1 g, 1.74 mmol, 2.0 eq) was added to above reaction mixture at 0° C. The resultant reaction mixture was stirred at room temperature for 16 h. Progress of the reaction was monitored by TLC. After complete consumption of starting materials, the reaction mixture was cooled to 0° C. and quenched with sat. NaHCO₃ solution (200 mL), extracted with DCM (2×200 mL). The combined organic layer was washed with water (100 mL) and brine (100 mL) dried over anhydrous Na₂SO₄, filtered, and concentrated under reduced pressure to afford tert-butyl 2-((1-(7-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)heptyl)piperidin-4-yl)methoxy)acetate (Int-7, 200 mg, 29%) as a colourless liquid.

### Step-8: Synthesis of 2-((1-(7-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)heptyl)piperidin-4-yl)methoxy)acetic acid (Int-8)

To a stirred solution of tert-butyl 2-((1-(7-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)heptyl)piperidin-4-yl)methoxy)acetate (Int-7, 300 mg 0.38 mmol, 1 eq) in 2,2,2-trifluoro ethanol (5 mL). Chlorotrimethylsilane (0.83 g, 7.62 mmol, 20 eq) was added at 0° C. The resultant reaction mixture was stirred at room temperature for 2 h. Progress of the reaction was monitored by TLC. After complete consumption of starting materials, the reaction mixture was evaporated under reduced pressure to obtain crude compound, which was washed with n-pentane-(5 mL) to afford 2-((1-(7-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)heptyl)piperidin-4-yl)methoxy)acetic acid (Int-8, 270 mg, 93%) as a colourless liquid.

### Step-9: Synthesis of (1S,9S)-1-(benzylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-9)

To a solution of ((1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (SM-4, 5 g, 9.40 mmol, 1.0 eq) in DMF (50 mL). DIPEA (4.9 mL, 28.20 mmol, 3.0 eq) was added at room temperature and stirred for 5 min. Benzyl bromide (1.6 g, 9.40 mmol, 1.0 eq) was added drop-wise and the resultant reaction mixture was stirred at room temperature for 16 h. Progress of the reaction was monitored by TLC. After complete consumption of starting material, the reaction mixture was poured into ice cold water (300 mL) and extracted with ethyl acetate (2×200 mL). The combined organic extracts were washed with brine (200 mL) and dried over anhydrous sodium sulphate, filtered and concentrated under reduced pressure to obtained crude compound, which was purified by combiflash chromatography by eluting with 0-70% ethyl acetate in heptane to afford (1S,9S)-1-(benzylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-9, 4.2 g, 85%) as a pale yellow solid.

### Step-10: Synthesis of (1S,9S)-1-(benzyl (methyl)amino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-10)

To a solution of (1S,9S)-1-(benzylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-9, 3 g, 5.70 mmol, 1.0 eq) in DCM (50 mL). Formaldehyde (22.3 mL, 28.54 mmol, 5.0 eq) and Formic acid (0.10 ml, 2.84 mmol, 0.5 eq) were added at 0° C. and the resultant reaction mixture was stirred at room temperature for 16 h. Progress of the reaction was monitored by TLC. After complete consumption of starting material, the reaction mixture was poured into ice cold water (200 mL) and extracted with DCM (2×100 mL). The combined organic extracts were washed with brine (100 mL) and dried over anhydrous sodium sulphate, filtered and concentrated under reduced pressure to obtain crude compound, which was purified by combiflash chromatography by eluting with 0-70% ethyl acetate in heptane to afford (1S,9S)-1-(benzyl (methyl)amino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-10, 2 g, 64%) as a pale brown solid.

### Step-11: Synthesis of (1S,9S)-9-ethyl-5-fluoro-9-hydroxy-4-methyl-1-(methylamino)-1,2,3,9,12,15-hexahydro-10H,13H-benzo[de]pyrano[3′,4′:6,7]indolizino[1,2-b]quinoline-10,13-dione (Int-A)

To a solution of (1S,9S)-1-(benzyl (methyl)amino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Int-10, 1.5 g, 2.80 mmol, 1.0 eq) in Acetonitrile (40 mL). CERIC AMMONIUM NITRATE (3 g, 5.60 mmol, 2.0 eq) in water (10 ml) was added at 0° C. and the resultant reaction mixture was stirred at room temperature for 16 h. Progress of the reaction was monitored by TLC. After complete consumption of starting materials, the reaction mixture was diluted with water (200 mL) and extracted with DCM (2×100 mL). The combined organic extracts were washed with brine (100 mL) and dried over anhydrous sodium sulphate, filtered and concentrated under reduced pressure to afford (1S,9S)-9-ethyl-5-fluoro-9-hydroxy-4-methyl-1-(methylamino)-1,2,3,9,12,15-hexahydro-10H,13H-benzo[de]pyrano[3′,4′:6,7]indolizino[1,2-b]quinoline-10,13-dione (Int-A, 1 g, 80%) as a pale brown solid.

### Step-12: Synthesis of (8S,11R,13S,14S,17R)-17-acetyl-11-(4-((7-(4-((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)(methyl)amino)-2-oxoethoxy)methyl)piperidin-1-yl) heptyl)(methyl)amino)phenyl)-13-methyl-3-oxo-2,3,6,7,8,11,12,13,14,15,16,17-dodecahydro-1H-cyclopenta[a]phenanthren-17-yl acetate

To a stirred solution of 2-((1-(7-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)heptyl)piperidin-4-yl)methoxy)acetic acid (Int-8, 280 mg, 0.38 mmol) in DMF (5 mL) was added 1-Propane phosphonic anhydride (50%) in ethyl acetate (0.36 g, 0.57 mmol, 1.5 eq) and N,N-Diisopropylethylamine (0.15 g, 1.14 mmol, 3.0 eq) at room temperature and stirred for 5 min. After that (1S,9S)-9-ethyl-5-fluoro-9-hydroxy-4-methyl-1-(methylamino)-1,2,3,9,12,15-hexahydro-10H,13H-benzo[de]pyrano[3′,4′:6,7]indolizino[1,2-b]quinoline-10,13-dione (Int-A, 172 mg, 0.38 mmol) was added at room temperature. The resultant reaction mixture was stirred at room temperature for 16 h. Progress of the reaction was monitored by TLC. After complete consumption of starting material, the reaction mixture was diluted with ice cold water (50 mL) and extracted with EtOAc (2×50 mL). The combined organic layer was washed with brine solution (50 mL), dried over anhydrous sodium sulphate, filtered, and concentrated under reduced pressure to obtain crude compound, which was purified by reverse phase purification (using 0.1% FA in water eluted in 55% Acetonitrile in 0.1% FA in water) to afford the title compound (85 mg, 19.09%) as an off white solid.

¹H NMR (400 MHz, DMSO-d₆) δ ppm 8.15 (s, 1H), 7.75-7.86 (m, 1H), 7.27-7.36 (m, 1H), 6.97 (br d, J=8.56 Hz, 2H), 6.58 (br d, J=8.68 Hz, 3H), 5.96-6.14 (m, 1H), 5.67 (s, 1H), 5.40 (s. 2H), 4.99 (br d, J=6.97 Hz, 2H), 4.10-4.54 (m, 3H), 3.44 (br d, J=5.38 Hz, 2H), 3.23 (br d, J=6.72 Hz, 4H), 3.00-3.17 (m, 3H), 2.81 (s, 4H), 2.70-2.76 (m, 3H), 2.67 (s, 2H), 2.55 (br s, 2H), 2.40 (s, 4H), 2.33-2.36 (m, 1H), 2.16-2.32 (m, 5H), 2.09-2.15 (m, 4H), 1.99 (s, 5H), 1.86 (br s, 3H), 1.55-1.78 (m, 5H), 1.31-1.50 (m, 8H), 1.26 (br s, 10H), 0.87 (br t, J=7.21 Hz, 5H); LCMS: 97.23%; ESI MS m/z calcd. For C₆₉H₈₄FN₅O₁₀ ([M+H]⁺) 1162.4; found 1161.9 HPLC: 96.03%.

### Example S56. Preparation of N-((1r,4r)-4-((3-chloro-4-cyanophenyl)(methyl)amino)cyclohexyl)-6-(4-(6-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)hexyl)piperidin-1-yl)pyridazine-3-carboxamide (Compound No. 56)

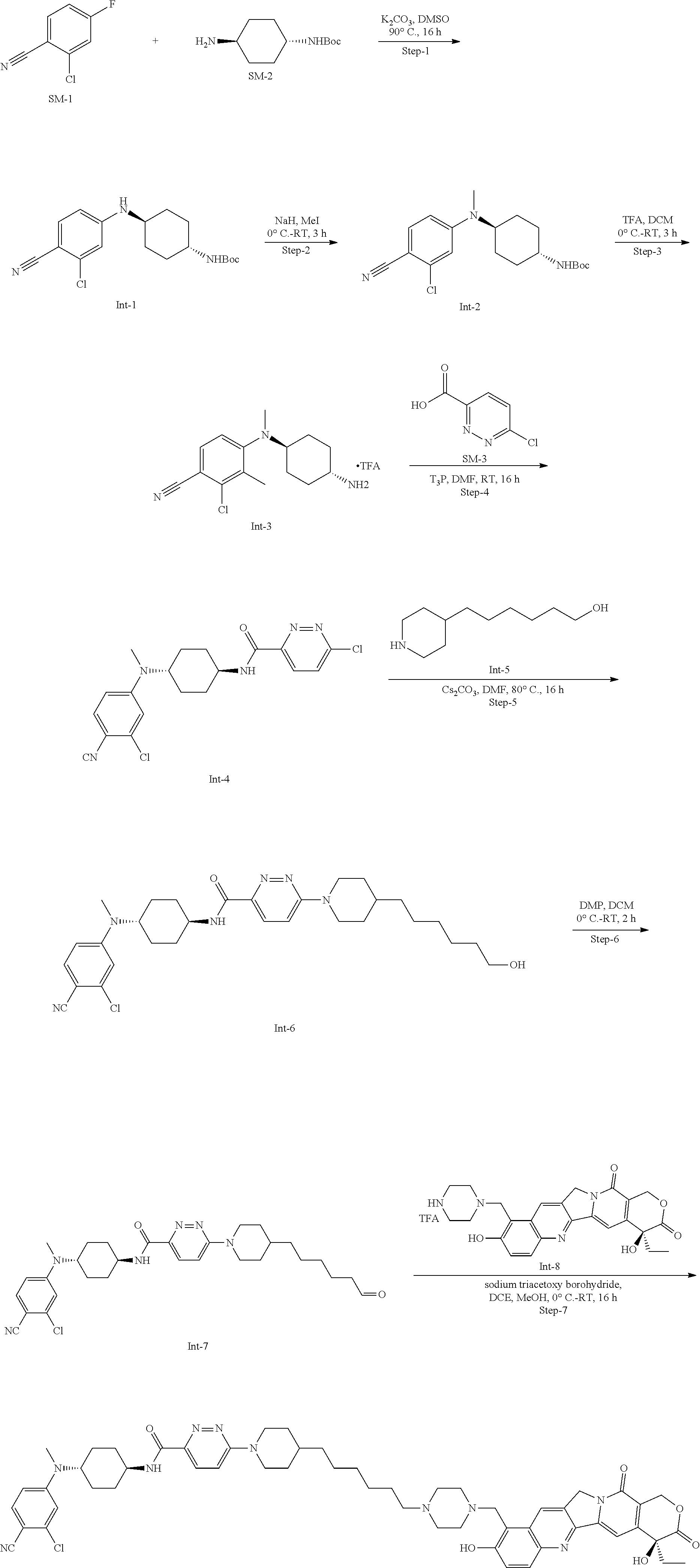

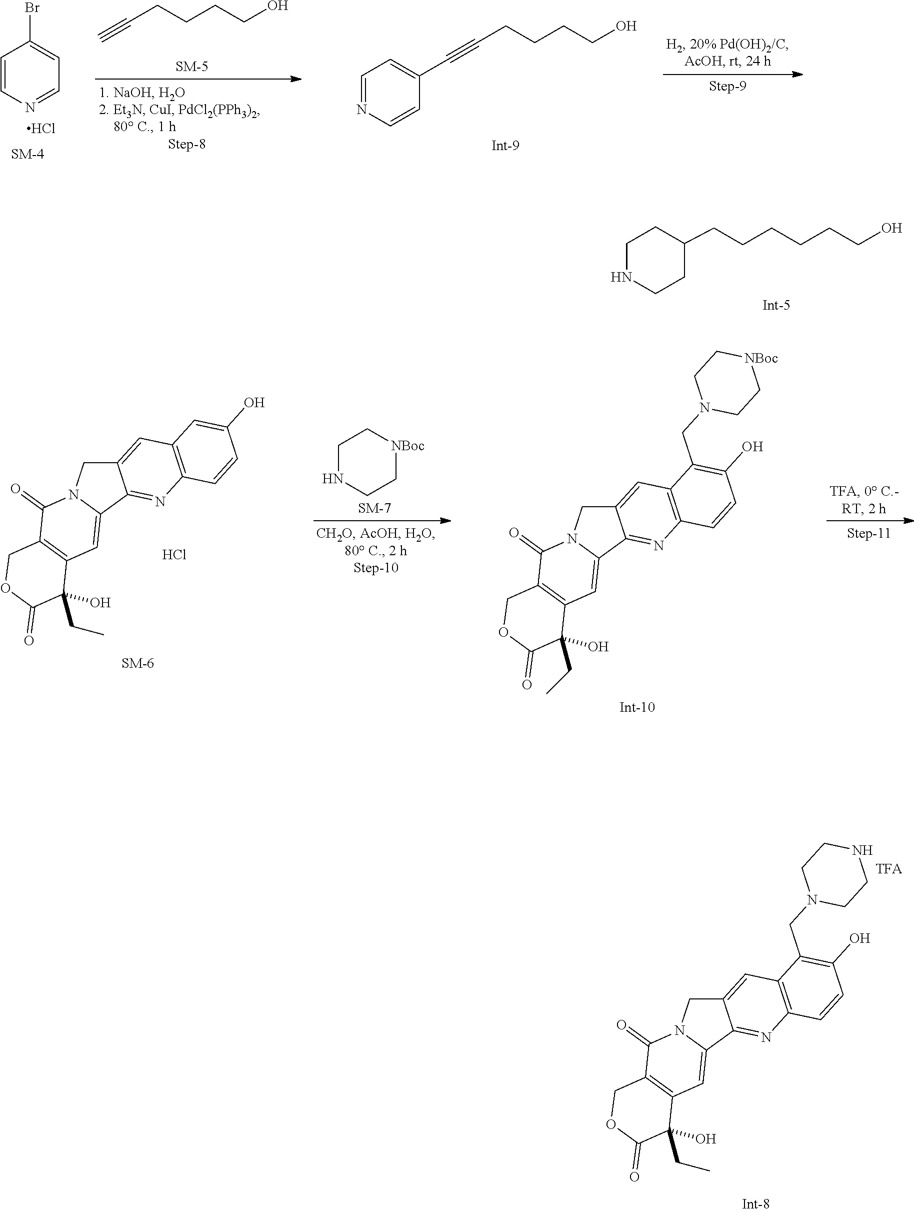

### Step-1: Synthesis of tert-butyl ((1r,4r)-4-((3-chloro-4-cyanophenyl)amino)cyclohexyl) carbamate (Int-1)

To a stirred solution of 2-chloro-4-fluorobenzonitrile (SM-1, 20 g, 128 mmol, 1.0 eq) in DMSO (200 mL) were added tert-butyl ((1r,4r)-4-aminocyclohexyl)carbamate (SM-2, 27.5 g, 128 mmol, 1.0 eq) and K₂CO₃ (35.3 g, 256 mmol, 2 eq) at room temperature. The resultant reaction mixture was heated to 90° C. for 16 h. Progress of the reaction was monitored by TLC. After complete consumption of the starting materials, the reaction mixture was diluted with ice cold water (1 L) and stirred for 10 min at room temperature. The obtained solids were filtered and washed with water, dried under vacuum. The crude solid compound was recrystallized by using EtOH to afford tert-butyl ((1r,4r)-4-((3-chloro-4-cyanophenyl)amino)cyclohexyl)carbamate (Int-1, 30 g, 67%) as an off white solid.

### Step-2: Synthesis of tert-butyl ((1r,4r)-4-((3-chloro-4-cyanophenyl)(methyl)amino) cyclohexyl)carbamate (Int-2)

To a stirred solution of tert-butyl ((1r,4r)-4-((3-chloro-4-cyanophenyl)amino)cyclohexyl) carbamate (Int-1, 30 g, 57.1 mmol, 1.0 eq) in DMF (300 mL) was added NaH (2.96 g, 74.3 mmol, 1.3 eq) under nitrogen atmosphere at 0° C. The resultant reaction mixture was allowed to room temperature and stirred for 30 min. then added methyl iodide (4.6 mL, 74.3 mmol, 1.3 eq) drop wise at 0° C. and allowed to room temperature, stirred for 3 h. Progress of the reaction was monitored by TLC. After complete consumption of starting material. The reaction mixture was diluted with ice cold water (1 L) and stirred for 10 min at room temperature. The obtained solids were filtered and washed with water, dried under vacuum. The obtained crude solid compound was recrystallized by using EtOH to afford tert-butyl ((1r,4r)-4-((3-chloro-4-cyanophenyl)(methyl)amino)cyclohexyl)carbamate (Int-2, 20 g, 64%) as an off white solid.

### Step-3: Synthesis of 4-(((1r,4r)-4-aminocyclohexyl)(methyl)amino)-2-chlorobenzonitrile. TFA Salt (Int-3)

To a stirred solution of tert-butyl ((1r,4r)-4-((3-chloro-4-cyanophenyl)(methyl)amino) cyclohexyl)carbamate (Int-2, 20 g, 54.9 mmol, 1.0 eq) in DCM (300 mL) was added TFA (100 mL, 5 vol) at 0° C. under nitrogen atmosphere. The resultant reaction mixture was allowed to room temperature and stirred for 3 h. Progress of the reaction was monitored by TLC. After complete consumption of starting material, the volatiles were evaporated under reduced pressure to obtain crude compound, which was washed with diethyl ether (20 mL) and dried under vacuum to afford 4-(((1r,4r)-4-aminocyclohexyl)(methyl)amino)-2-chlorobenzonitrilo (Int-3, 15 g. crude) as an off white solid.

### Step-4: Synthesis of 6-chloro-N-((1r,4r)-4-((3-chloro-4-cyanophenyl)(methyl)amino) cyclohexyl)pyridazine-3-carboxamide (Int-4)

To a stirred solution of 4-(((1r,4r)-4-aminocyclohexyl)(methyl)amino)-2-chlorobenzonitrile·TFA salt (Int-3, 15 g, 57.03 mmol, 1.0 eq) and 6-chloropyridazine-3-carboxylic acid (SM-3, 9.04 g, 57.03 mmol, 1.0 eq) in DMF (150 mL), T3P (36 g, 114 mmol, 2 eq) and DIPEA (30 mL, 171 mmol, 3 eq) were added at room temperature and stirred for 16 h. Progress of the reaction was monitored by TLC. After completion of the reaction, quench with ice cold water (1 L) to reaction and stirred for 10 min at room temperature, obtained solids were filtered and washed water, dried under vacuum afford 6-chloro-N-((1r,4r)-4-((3-chloro-4-cyanophenyl)(methyl)amino)cyclohexyl)pyridazine-3-carboxamide (Int-4, 13 g, 56%) as an off white solid,

### Step-5: Synthesis of N-((1r,4r)-4-((3-chloro-4-cyanophenyl)(methyl)amino)cyclohexyl)-6-(4-(6-hydroxyhexyl)piperidin-1-yl)pyridazine-3-carboxamide (Int-6)

To a stirred solution of 6-chloro-N-((1r,4r)-4-((3-chloro-4-cyanophenyl)(methyl)amino) cyclohexyl)pyridazine-3-carboxamide (Int-4, 800 mg, 2 mmol) and 6-(piperidin-4-yl)hexan-1-ol (Int-5, 400 mg, 2 mmol) in DMF (10 mL) was added potassium Iodide (71 mg, 0.43 mmol), Cs₂CO₃ (2 g, 6 mmol) at room temperature and the reaction mixture was heated to 80° C. and stirred for 16 h. Progress of the reaction was monitored by TLC/LCMS. After completion, the reaction mixture was diluted with water (50 mL) and extracted with EtOAc (2×30 mL). The combined organic extracts were washed with water (30 mL) and brine (15 mL), dried over anhydrous sodium sulphate, filtered, and concentrated under vacuum to afford crude product which was purified by combi-flash column chromatography by eluting with 60-65% ethyl acetate in n-heptane to afford N-((1r,4r)-4-((3-chloro-4-cyanophenyl)(methyl)amino)cyclohexyl)-6-(4-(6-hydroxyhexyl)piperidin-1-yl)pyridazine-3-carboxamide (Int-6, 180 mg, 20%) as an off-white solid.

### Step-6: Synthesis of N-((1r,4r)-4-((3-chloro-4-cyanophenyl)(methyl)amino)cyclohexyl)-6-(4-(6-oxohexyl)piperidin-1-yl)pyridazine-3-carboxamide (Int-7)

A stirred solution of N-((1r,4r)-4-((3-chloro-4-cyanophenyl)(methyl)amino)cyclohexyl)-6-(4-(6-hydroxyhexyl)piperidin-1-yl)pyridazine-3-carboxamide (Int-6, 180 mg, 0.32 mmol) in DCM (10 mL). Dess-Martin periodinane (DMP) (165 mg, 0.39 mmol) was added portion wise at 0° C. and the reaction mixture was stirred at ambient temperatures for 2 h. Progress of the reaction was monitored by TLC. After complete completion of starting material, the reaction mixture was quenched with 50% aqueous Na₂S₂O₃ solution (10 mL), sat. NaHCO₃ solution (30 mL) and extracted with DCM (2×30 mL). The combined organic extracts were washed with brine (30 mL) and dried over anhydrous sodium sulphate, filtered and concentrated under vacuum to afford N-((1r,4r)-4-((3-chloro-4-cyanophenyl)(methyl)amino)cyclohexyl)-6-(4-(6-oxohexyl)piperidin-1-yl)pyridazine-3-carboxamide (Int-7, 130 mg, 30%) as an off-white solid.

### Step-7: Synthesis of N-((1r,4r)-4-((3-chloro-4-cyanophenyl)(methyl)amino)cyclohexyl)-6-(4-(6-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)hexyl)piperidin-1-yl)pyridazine-3-carboxamide

To a stirred solution of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFA (Int-8, 130 mg, 0.23 mmol) in DCE (5 mL) and MeOH (1 mL) were added N-((1r,4r)-4-((3-chloro-4-cyanophenyl)(methyl)amino)cyclohexyl)-6-(4-(6-oxohexyl)piperidin-1-yl)pyridazine-3-carboxamide (Int-7, 120 mg, 0.25 mmol) and Et₃N (0.09 mL, 0.7 mmol) at 0° C. The resultant mixture was stirred at room temperature for 2 h. After that sodium triacetoxy borohydride (99 mg, 0.47 mmol) and acetic acid (0.1 mL, 0.2 mmol) were added at 0° C. The resultant reaction mixture was stirred at room temperature for 16 h Progress of the reaction was monitored by TLC. After complete consumption of starting materials, the reaction mass was cooled to 0° C. and quenched with sat. NaHCO₃ solution (10 mL), extracted with 10% MeOH in DCM (2×50 mL). The combined organic layer was washed with water (20 mL) and brine (20 mL), dried over anhydrous Na₂SO₄ and concentrated under reduced pressure to obtain crude product as a pale brown solid, which was purified by reverse phase (using 0.1% FA in water eluted in 55%

Acetonitrile in 0.1% FA in water) to afford N-((1r,4r)-4-((3-chloro-4-cyanophenyl)(methyl)amino)cyclohexyl)-6-(4-(6-(4-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperazin-1-yl)hexyl)piperidin-1-yl)pyridazine-3-carboxamide (21 mg, 8.2%) as a pale yellow solid.

¹H NMR (400 MHz, DMSO-d₆): δ ppm 8.72 (s, 1H), 8.46 (d, J=8.44 Hz, 1H), 8.16 (s, 1H), 7.98 (d, J=9.05 Hz, 1H), 7.79 (d, J=9.66 Hz, 1H), 7.60 (d, J=8.93 Hz, 1H), 7.41 (d, J=9.17 Hz, 1H), 7.32 (d, J=9.78 Hz, 1H), 7.26 (s, 1H), 6.94 (d, J=2.45 Hz, 1H), 6.82 (dd, J=9.05, 2.45 Hz, 1H), 6.45-6.51 (m, 1H), 5.41 (s, 2H), 5.25 (s, 2H), 4.47 (d, J=13.08 Hz, 2H), 4.10 (s, 2H), 3.78-3.87 (m, 2H), 3.41 (s, 2H), 3.17 (s, 1H), 2.98 (d, J=11.74 Hz, 2H), 2.85 (s, 3H), 2.56-2.64 (m, 2H), 2.24-2.30 (m, 2H), 1.82-1.94 (m, 4H), 1.75 (d, J=11.98 Hz, 4H), 1.61-1.71 (m, 5H), 1.50-1.57 (m, 1H), 1.36-1.46 (m, 2H), 1.23-1.32 (m, 9H), 1.09 (t, J=7.03 Hz, 3H), 0.88 (t, J=7.34 Hz, 3H); ESI MS m/z calcd. For C₅₅H₆₅ClN₁₀O₆ ([M+H]⁺) 997.4; found 997.4.

### Step-8: Synthesis of 6-(pyridin-4-yl)hex-5-yn-1-ol (Int-9)

A solution of 4-bromopyridine. HCl salt (SM-4, 5.0 g, 25.77 mmol) in EtOAc (250 mL) was cooled to 0° C. followed by addition of aqueous sodium hydroxide solution (10.3 g, 257.7 mmol) in water (100 mL) and then the reaction mixture was allowed to stir at room temperature for 30 minutes. After consumption of (SM-4) as monitored by TLC, both the layers were separated, and organic layer was washed with brine (100 mL) solution, dried over anhydrous Na₂SO₄, filtered, and concentrated under reduced pressure to give 4-bromopyridine (3.50 g, 86%, crude, HCl salt free) as colourless liquid.

To a stirred solution of above prepared 4-bromopyridine (3.5 g, 22.29 mmol) and hex-5-yn-1-ol (SM-5, 2.18 g, 22.29 mmol) in triethyl amine (10 mL) were added copper iodide (84.7 mg, 0.44 mmol) and PdCl₂(PPh₃)₂ (156 mg, 0.22 mmol) at room temperature and the reaction mixture was heated to 80° C. and stirred for 1 h. Progress of the reaction was monitored by TLC and LCMS. After complete consumption of starting materials, the reaction mixture was diluted with water (150 mL) and extracted with EtOAc (2×80 mL). The combined organic layers were washed with brine (80 mL), water (75 mL) and dried over anhydrous Na₂SO₄, filtered and concentrated under reduced pressure to give crude product, which was purified by Combiflash column chromatography by eluting with 70% EtOAc in heptane to afford 6-(pyridin-4-yl)hex-5-yn-1-ol (Int-9, 3.8 g, 97%) as a light-yellow oil.

### Step-9: Synthesis of 6-(piperidin-4-yl)hexan-1-ol (Int-5)

To a stirred solution of 6-(pyridin-4-yl)hex-5-yn-1-ol (Int-9, 3.8 g, 21.7 mmol) and acetic acid (80 mL) in autoclave was added 20% Pd(OH)₂/C (1.60 g). Filled with H₂ gas (100 PSI) and the resultant reaction mixture was allowed to stir at ambient temperature for 24 h. Progress of the reaction was monitored by TLC. After complete consumption of starting materials, the reaction mixture was filtered through a celite bed and washed with EtOAc (100 mL). The combined filtrate was concentrated under reduced pressure to afford 6-(piperidin-4-yl)hexan-1-ol (Int-5, 3.5 g, 87%) as a brown oil, which was used as such for next step without any further purification.

### Step-10: Synthesis of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-10)

To a stirred solution of (S)-4-ethyl-4,9-dihydroxy-1,12-dihydro-14H-pyrano[3′,4′:6,7]indolizino[1,2-b]quinoline-3,14(4H)-dione·HCl salt (SM-6, 2.5 g, 6.86 mmol, 1.0 eq) and tert-butyl piperazine-1-carboxylate (SM-7, 1.85 g, 10.3 mmol, 1.5 eq) in acetic acid (10 mL) under inert atmosphere was added 37% solution of formaldehyde (0.29 mL, 8.24 mmol, 1.2 eq) at room temperature. The resultant reaction mixture was heated to 80° C. and stirred for 2 h in a sealed tube. Progress of the reaction was monitored by TLC. After complete consumption of starting materials, the volatiles were evaporated under reduced pressure to obtain crude compound, which was basified with aq. ammonia until the pH reached to 9, filtered the precipitated solid and washed with water (10 mL) and dried under vacuum to afford tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1-carboxylate (Int-10, 1.9 g, 50%) as a yellow solid.

### Step-11: Synthesis of (S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione·TFAsalt (Int-8)

To a stirred solution of tert-butyl (S)-4-((4-ethyl-4,9-dihydroxy-3,14-dioxo-3,4,12,14-tetrahydro-1H-pyrano[3′,4′:6,7]indolizino[1,2-b]quinolin-10-yl)methyl)piperazine-1 carboxylate (Int-10, 2 g, 3.5 mmol, 1.0 eq) in DCM (20 mL) under nitrogen atmosphere was added TFA (2.7 mL, 35 mmol, 10 eq) at 0° C. The resultant reaction mixture was warmed to room temperature and stirred for 2 h. Progress of the reaction was monitored by TLC. After complete consumption of starting materials, the volatiles were evaporated under reduced pressure to obtain crude compound, which was washed with diethyl ether (10 mL) and dried under vacuum to afford(S)-4-ethyl-4,9-dihydroxy-10-(piperazin-1-ylmethyl)-1,12-dihydro-14H-pyrano[3′,4′:6,7]indolizino[1,2-b]quinoline-3,14(4H)-dione (Int-8, 1.4 g, 83%) as a yellow solid.

### Example S57. Preparation of (S)-4-ethyl-9-hydroxy-3,14-dioxo-3,4,12,14-tetrahydro-1H-pyrano[3′,4′:6,7]indolizino[1,2-b]quinolin-4-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Compound No. 57)

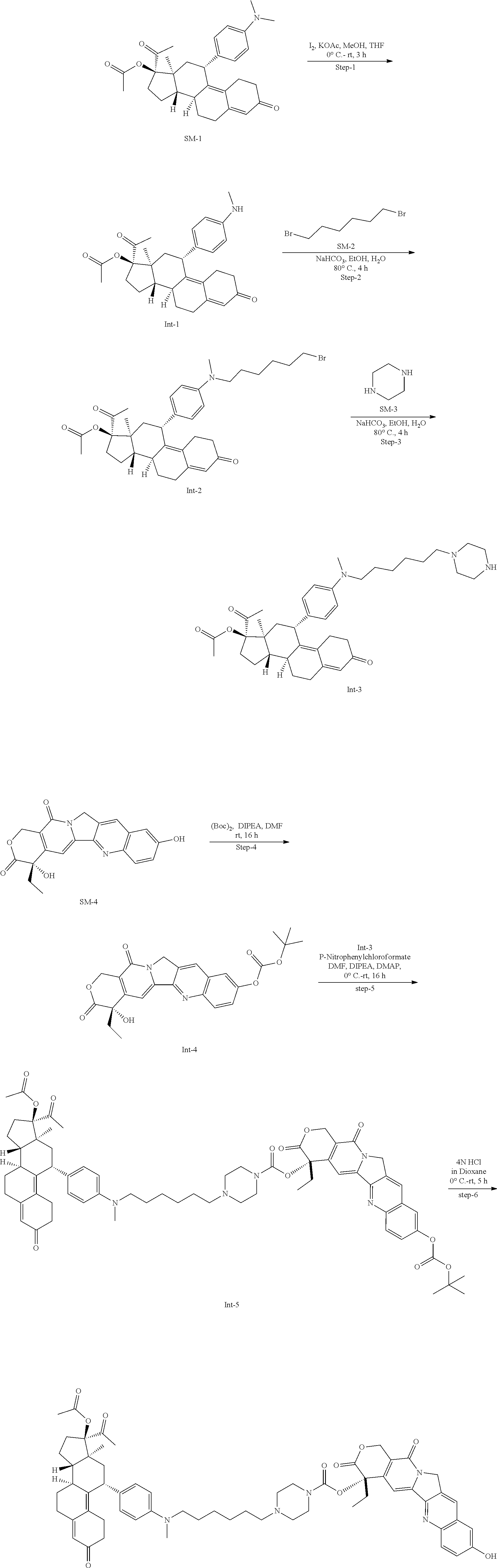

### Step-1: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-1, 15 g, 31.57 mmol, 1.0 eq) in methanol (75 mL) and THF (150 mL) were added KOAc (30.99 g, 315 mmol, 10 eq) and Iodine (20.33 g, 157 mmol, 5 eq) at 0° C. The resultant reaction mixture was allowed to room temperature and stirred for 3 h. Progress of the reaction was monitored by TLC. After complete consumption of the starting materials, the reaction mixture was quenched with sodium thiosulfate (Na₂S₂O₃) solution (70 g in 45 mL water) and extracted with ethyl acetate (2×300 mL). The combined organic extracts were washed with brine (150 mL) and dried over anhydrous Na₂SO₄, filtered, and concentrated under reduced pressure to obtain (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 9.5 g, 65%) as an off-white solid, which was used in next step without any further purification.

### Step-2: Synthesis of (8S,11R,13S,14S,17R)-17-acetyl-11-(4-((6-bromohexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 8.5 g, 18.43 mmol, 1.0 eq) and 1,6-dibromohexane (SM-2, 22.47 g, 92.19 mmol, 5 eq) in ethanol (85 mL) and water (28 mL) was added NaHCO₃ (4.6 g, 55.31 mmol, 3 eq) at room temperature. The resultant reaction mixture was heated to 80° C. and stirred for 4 h. Progress of the reaction was monitored by TLC. After complete consumption of the starting materials, the reaction mixture was filtered through a pad of celite bed and washed with ethyl acetate (85 mL). Filtrate was concentrated under reduced pressure, diluted with water (240 mL) and extracted with ethyl acetate (2×400 mL), The combined organic extracts were washed with brine (200 mL) and dried over anhydrous Na₂SO₄, filtered, and concentrated under reduced pressure to obtain crude compound, which was purified by combi flash chromatography (by eluting with 70% ethyl acetate in heptane) to afford (8S,11R,13S,14S,17R)-17-acetyl-11-(4-((6-bromohexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 4 g, 34%) as an off-white solid.

### Step-3: Synthesis of (8S,11R,13S,14S,17R)-17-acetyl-13-methyl-11-(4-(methyl(6-(piperazin-1-yl)hexyl)amino)phenyl)-3-oxo-2,3,6,7,8,11,12,13,14,15,16,17-dodecahydro-1H-cyclopenta[a]phenanthren-17-yl acetate (Int-3)

To a stirred solution of (8S,11R,13S,14S,17R)-17-acetyl-11-(4-((6-bromohexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 4.0 g, 6.410 mmol, 1.0 eq) and piperazine (SM-3, 2.75 g, 32.05 mmol, 5 eq) in ethanol (80 mL) and water (26 mL) was added NaHCO₃ (1.61 g, 26.03 mmol, 3 eq) at room temperature. The reaction mixture was stirred at 80° C. for 4 h. Progress of the reaction was monitored by TLC. After complete consumption of the starting materials, the reaction mixture was filtered through a pad of celite bed and washed with ethyl acetate (85 mL), Filtrate was concentrated under reduced pressure and the obtained crude materials was diluted with water (240 mL) and extracted with ethyl acetate (2×400 mL). The combined organic extracts were washed with brine (200 mL) and dried over anhydrous Na₂SO₄, filtered, and concentrated under reduced pressure to obtain crude compound, which was purified by combi flash chromatography by eluting with 0-70% ethyl acetate in heptane to afford (8S,11R,13S,14S,17R)-17-acetyl-13-methyl-11-(4-(methyl(6-(piperazin-1-yl)hexyl)amino)phenyl)-3-oxo-2,3,6,7,8,11,12,13,14,15,16,17-dodecahydro-1H-cyclopenta[a]phenanthren-17-yl acetate (Int-3, 1.2 g, 30%) as an off-white solid.

### Step-4: Synthesis of (S)-tert-butyl (4-ethyl-4-hydroxy-3,14-dioxo-3,4,12,14-tetrahydro-1H-pyrano[3′,4′:6,7]indolizino[1,2-b]quinolin-9-yl)carbonate (Int-4)

To a stirred solution of ((S)-4-ethyl-4,9-dihydroxy-1,12-dihydro-14H-pyrano[3′,4′:6,7]indolizino[1,2-b]quinoline-3,14(4H)-dione (SM-4, 1 g, 2.74 mmol, 1.0 eq) in DMF (34 mL). DIPEA (11.2 g, 142 mmol, 51 eq) and Boc anhydride (1.19 g, 5.49 mmol, 2 eq) were added at 0° C. The resultant reaction mixture was allowed to room temperature and stirred for 16 h. Progress of the reaction was monitored by TLC. After complete consumption of starting materials, the reaction mixture was diluted with water (300 ml) and extracted with ethyl acetate (2×100 mL). The combined organic extracts were washed with 1N HCl (100 ml) and brine (100 mL), dried over anhydrous Na₂SO₄, filtered, and concentrated under reduced pressure to afford(S)-tert-butyl (4-ethyl-4-hydroxy-3,14-dioxo-3,4,12,14-tetrahydro-1H-pyrano[3′,4′:6,7]indolizino[1,2-b]quinolin-9-yl)carbonate (Int-4, 1.0 g, 78%) as an orange solid which was used in next step without any further purification.

### Step-5: Synthesis of (S)-9-((tert-butoxycarbonyl)oxy)-4-ethyl-3,14-dioxo-3,4,12,14-tetrahydro-1H-pyrano[3′,4′:6,7]indolizino[1,2-b]quinolin-4-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Int-5)

To the stirred solution of (S)-tert-butyl (4-ethyl-4-hydroxy-3,14-dioxo-3,4,12,14-tetrahydro-1H-pyrano[3′,4′:6,7]indolizino[1,2-b]quinolin-9-yl)carbonate (Int-4, 250 mg, 0.53 mmol, 1.0 eq) in DCM (5 mL). DIPEA (210 mg, 1.61 mmol, 3 eq). DMAP (190 mg, 1.61 mmol, 3 eq) and P-Nitrophenylchloroformate (130 mg, 0.64 mmol, 1.2 eq) at 0° C. and stirred for 1 h. Then (8S,11R,13S,14S,17R)-17-acetyl-13-methyl-11-(4-(methyl(6-(piperazin-1-yl)hexyl)amino)phenyl)-3-oxo-2,3,6,7,8,11,12,13,14,15,16,17-dodecahydro-1H-cyclopenta[a]phenanthren-17-yl acetate (Int-3, 339 mg, 0.53 mmol, 1.0 eq) was added and the resultant reaction mixture was allowed to room temperature and stirred for 16 h. Progress of the reaction was monitored by TLC. After complete consumption of starting materials, the reaction mixture was diluted with water (60 ml) and extracted with ethyl acetate (2×50 mL). The combined organic extracts were washed with brine (50 mL) and dried over anhydrous Na₂SO₄, filtered, and concentrated under reduced pressure to obtain crude compound, which was purified by combi-flash chromatography by eluting with 0-30% ethyl acetate in heptane to afford(S)-9-((tert-butoxycarbonyl)oxy)-4-ethyl-3,14-dioxo-3,4,12,14-tetrahydro-1H-pyrano[3′,4′:6,7]indolizino[1,2-b]quinolin-4-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Int-5, 250 mg, 41%) as an orange solid.

### Step-6: Synthesis of (S)-4-ethyl-9-hydroxy-3,14-dioxo-3,4,12,14-tetrahydro-1H-pyrano[3′,4′:6,7]indolizino[1,2-b]quinolin-4-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate

To a stirred solution of (S)-9-((tert-butoxycarbonyl)oxy)-4-ethyl-3,14-dioxo-3,4,12,14-tetrahydro-1H-pyrano[3′,4′:6,7]indolizino[1,2-b]quinolin-4-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)piperazine-1-carboxylate (Int-5, 250 mg, 0.22 mmol, 1 eq) in dichloromethane (2.5 mL), 4M HCl in 1,4-Dioxane (0.7 ml) was added at 0° C. and the resultant reaction mixture was stirred under nitrogen atmosphere at ambient temperature for 5 h. Progress of the reaction was monitored by TLC. After complete consumption of starting materials, the reaction mixture was concentrated under reduced pressure to obtain crude compound which was purified by reverse phase purification (using 0.1% FA in water eluted in 55% Acetonitrile in 0.1% FA in water) to afford the title compound (66 mg, 29%) as an off-white solid.

¹H NMR (400 MHz, DMSO-d₆) δ ppm 10.14-10.44 (m, 1H), 8.25 (s, 1H), 8.22 (s, 1H), 7.76 (d, J=9.26 Hz, 1H), 7.18 (dd, J=9.13, 2.63 Hz, 1H), 7.06 (d, J=2.63 Hz, 1H), 6.65-6.81 (m, 3H), 6.35 (d, J=8.76 Hz, 2H), 5.43 (s, 1H), 5.20 (d, J=3.63 Hz, 2H), 5.02 (s, 2H), 4.16 (br d, J=6.50 Hz, 1H), 3.46-3.63 (m, 1H), 3.25-3.41 (m, 1H), 2.91-3.03 (m, 5H), 2.48-2.62 (m, 4H), 2.42-2.46 (m, 1H), 2.07-2.16 (m, 3H), 2.02-2.07 (m, 3H), 1.97 (br d, J=4.88 Hz, 1H), 1.89-1.94 (m, 4H), 1.87 (s, 4H), 1.55-1.77 (m, 5H), 1.40-1.54 (m, 2H), 1.13-1.26 (m, 5H), 1.00-1.09 (m, 5H), 0.68 (t, J=7.38 Hz, 3H); LCMS: 93.39%; ESI MS m/z calcd. For C₆₀H₆₉N₅O₁₀ ([M+H]⁺) 1020.2; found 1020.6 HPLC: 93.54%.

### Example S58. Preparation of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((1-(4-(3-(3-chloro-4-cyano phenyl)-5,5-dimethyl-4-oxo-2-thioxoimidazolidin-1-yl)phenyl)piperidin-4-yl)methyl)piperazine-1-carboxylate (Compound No. 58)

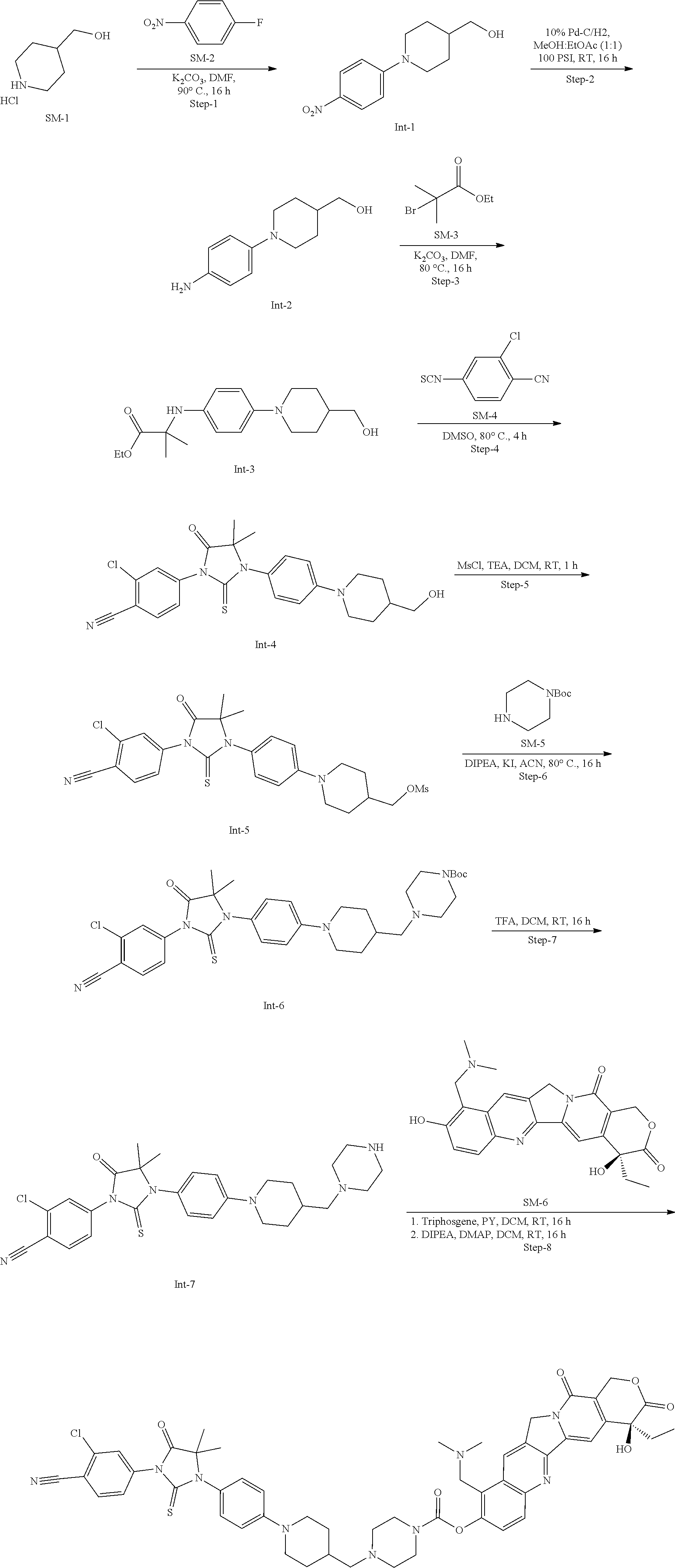

### Step-1: Synthesis of (1-(4-nitrophenyl)piperidin-4-yl)methanol (Int-1)

To a stirred solution of piperidin-4-ylmethanol·HCl (SM-1, 30 g, 197.8 mmol, 1.0 eq) in DMF (600 mL) were added 1-fluoro-4-nitrobenzene (SM-2, 33.4 g, 237.4 mmol, 1.2 eq) and K₂CO₃ (81.9 g, 593.5 mmol, 3.0 eq) at 0° C. under argon atmosphere. The resulting reaction mixture was stirred at 90° C. for 16 h. Progress of the reaction was monitored by TLC. After complete consumption of starting materials, the reaction mixture was diluted with water (1 L) and filtered the obtained solids, washed with water (500 mL) and dried under vacuum to afford (1-(4-nitrophenyl)piperidin-4-yl)methanol (Int-1, 40 g, 87%) as a pale yellow solid.

### Step-2: Synthesis of (1-(4-aminophenyl)piperidin-4-yl)methanol (Int-2)

To a stirred solution of (1-(4-nitrophenyl)piperidin-4-yl)methanol (Int-1, 10 g, 42.3 mmol, 1.0 eq) in MeOH:EtOAc (1:1, 300 mL) was added 10% Pd—C (2 g, 20% w/w) at ambient temperature, under argon atmosphere. The resultant reaction mixture was stirred under Hydrogen atmosphere (100 PSI) for 16 h (Autoclave). Progress of the reaction was monitored by TLC. After complete consumption of starting materials, the reaction mixture was filtered through celite bed and washed with MeOH (200 mL), the obtained filtrate was evaporated under reduced pressure to afford crude (1-(4-aminophenyl)piperidin-4-yl)methanol (Int-2, 10 g) as a pale yellow gummy liquid.

### Step-3: Synthesis of ethyl 2-((4-(4-(hydroxymethyl)piperidin-1-yl)phenyl)amino)-2-methylpropanoate (Int-3)

To a stirred solution of (1-(4-aminophenyl)piperidin-4-yl)methanol (Int-2, 10 g, 48.54 mmol, 1.0 eq) in DMF (200 mL) were added ethyl 2-bromo-2-methylpropanoate (SM-3, 11.35 g, 58.25 mmol, 1.2 eq) and K₂CO₃ (20.09 g, 145.63 mmol, 3.0 eq) at 0° C. under argon atmosphere. The resulting reaction mixture was stirred at 80° C. for 16 h. Progress of the reaction was monitored by TLC. After complete consumption of starting materials, the reaction mixture was diluted with water (500 mL) and extracted with ethyl acetate (2×500 mL). The combined organic layer was washed with water (200 mL), brine (200 mL) and dried over anhydrous sodium sulphate, filtered and concentrated under reduced pressure to obtain crude compound, which was purified by flash column chromatography to afford ethyl 2-((4-(4-(hydroxymethyl)piperidin-1-yl)phenyl)amino)-2-methylpropanoate (Int-3, 10 g, 64%) as a pale yellow solid.

### Step-4: Synthesis of 2-chloro-4-(3-(4-(4-(hydroxymethyl)piperidin-1-yl)phenyl)-4,4-dimethyl-5-oxo-2-thioxoimidazolidin-1-yl)benzonitrile (Int-4)

To a stirred solution of ethyl 2-((4-(4-(hydroxymethyl)piperidin-1-yl)phenyl)amino)-2-methylpropanoate (Int-3, 30 g, 93.75 mmol, 1.0 eq) in DMSO (120 mL) was added 2-chloro-4-isothiocyanatobenzonitrile (SM-4, 21.8 g, 112.5 mmol, 1.2 eq) at 0° C. under argon atmosphere. The resultant reaction mixture was stirred at 80° C. for 4 h. Progress of the reaction was monitored by TLC. After complete consumption of starting materials, the reaction mixture was diluted with water (500 mL) and filtered the obtained solids, washed with water (500 mL) and diethyl ether (300 mL), dried under vacuum to afford 2-chloro-4-(3-(4-(4-(hydroxymethyl)piperidin-1-yl)phenyl)-4,4-dimethyl-5-oxo-2-thioxoimidazolidin-1-yl)benzonitrile (Int-4, 35 g, 81%) as a pale yellow solid.

### Step-5: Synthesis of (1-(4-(3-(3-chloro-4-cyanophenyl)-5,5-dimethyl-4-oxo-2-thioxoimidazolidin-1-yl)phenyl)piperidin-4-yl)methyl methanesulfonate (Int-5)

To a stirred solution of 2-chloro-4-(3-(4-(4-(hydroxymethyl)piperidin-1-yl)phenyl)-4,4-dimethyl-5-oxo-2-thioxoimidazolidin-1-yl)benzonitrile (Int-4, 10 g, 21.36 mmol, 1.0 eq) in DCM (300 mL), were added MsCl (1.81 mL, 23.5 mmol, 1.1 eq) and TEA (6 mL, 42.73 mmol, 2.0 eq) at 0° C. under argon atmosphere. The resulting reaction mixture was stirred at RT for 1 h. Progress of the reaction was monitored by TLC. After complete consumption of starting materials, the reaction mixture was dilute with water (500 mL) and extracted with DCM (2×500 mL). The combined organic layer was washed with brine (200 mL), dried over anhydrous sodium sulphate, filtered and evaporated under reduced pressure to afford crude (1-(4-(3-(3-chloro-4-cyanophenyl)-5,5-dimethyl-4-oxo-2-thioxoimidazolidin-1-yl)phenyl)piperidin-4-yl)methyl methanesulfonate (Int-5, 11.7 g) as a yellow gummy liquid.

### Step-6: Synthesis of tert-butyl 4-((1-(4-(3-(3-chloro-4-cyanophenyl)-5,5-dimethyl-4-oxo-2-thioxoimidazolidin-1-yl)phenyl)piperidin-4-yl)methyl)piperazine-1-carboxylate (Int-6)

To a stirred solution of (1-(4-(3-(3-chloro-4-cyanophenyl)-5,5-dimethyl-4-oxo-2-thioxoimidazolidin-1-yl)phenyl)piperidin-4-yl)methyl methanesulfonate (Int-5, 11.7 g, 21.48 mmol, 1.0 eq) in Acetonitrile (120 mL were added tert-butyl piperazine-1-carboxylate (SM-5, 4 g, 21.48 mmol, 1.0 eq), DIPEA (7.5 mL, 42.96 mmol, 2.0 eq) and KI (350 mg, 2.14 mmol, 0.1 eq) at 0° C. under argon atmosphere. The resulting reaction mixture was stirred at 80° C. for 16 h. Progress of the reaction was monitored by TLC. After complete consumption of starting materials, the volatiles were removed under reduced pressure and dilute with water (200 mL), followed by extracted with ethyl acetate (2×200 mL). The combined organic layer was washed with brine (100 mL), dried over anhydrous sodium sulphate, filtered and concentrated under reduced pressure to obtain crude compound, which was purified by flash column chromatography to afford tert-butyl 4-((1-(4-(3-(3-chloro-4-cyanophenyl)-5,5-dimethyl-4-oxo-2-thioxoimidazolidin-1-yl)phenyl)piperidin-4-yl)methyl)piperazine-1-carboxylate (Int-6, 10 g, 73%) as an off white solid.

### Step-7: Synthesis of 2-chloro-4-(4,4-dimethyl-5-oxo-3-(4-(4-(piperazin-1-ylmethyl)piperidin-1-yl)phenyl)-2-thioxoimidazolidin-1-yl)benzonitrile (Int-7)

To a stirred solution of tert-butyl 4-((1-(4-(3-(3-chloro-4-cyanophenyl)-5,5-dimethyl-4-oxo-2-thioxoimidazolidin-1-yl)phenyl)piperidin-4-yl)methyl)piperazine-1-carboxylate (Int-6, 5.2 g, 8.16 mmol, 1.0 eq) in DCM (104 mL) was added TFA (26 mL, 5 vol) at 0° C. under argon atmosphere. The resulting reaction mixture was stirred at RT for 16 h. Progress of the reaction was monitored by TLC. After complete consumption of starting materials, the volatiles were removed under reduced pressure and diluted with water (100 mL), followed by washed with diethyl ether (2 100 mL). The aqueous layer was basified with saturated bicarbonate solution (200 ml. pH up to ˜8) and filtered the obtained solids, washed with water (500 mL) and diethyl ether (300 mL), dried under vacuum to afford 2-chloro-4-(4,4-dimethyl-5-oxo-3-(4-(4-(piperazin-1-ylmethyl)piperidin-1-yl)phenyl)-2-thioxoimidazolidin-1-yl)benzonitrile (Int-7, 4 g, 93%) as an off white solid.

### Step-8: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-((1-(4-(3-(3-chloro-4-cyano phenyl)-5,5-dimethyl-4-oxo-2-thioxoimidazolidin-1-yl)phenyl)piperidin-4-yl)methyl)piperazine-1-carboxylate

To a stirred solution of 2-chloro-4-(4,4-dimethyl-5-oxo-3-(4-(4-(piperazin-1-ylmethyl)piperidin-1-yl)phenyl)-2-thioxoimidazolidin-1-yl)benzonitrile (Int-7, 500 mg, 0.9 mmol, 1.0 eq) in DCM (5 mL) were added Triphosgene (140 mg, 0.45 mmol) and Pyridine (185 mg, 2.3 mmol) at 0° C. under argon atmosphere. The resulting reaction mixture was stirred at RT for 2 h. Progress of the reaction was monitored by TLC. After complete consumption of starting materials, the reaction mixture was diluted with water (100 mL) and extracted with DCM (2×100 mL). The combined organic layer was washed with water (100 mL) and brine (100 mL), dried over anhydrous Na₂SO₄, filtered and concentrated under reduced pressure to obtain crude compound (500 mg, Crude). To a stirred solution of above crude compound (500 mg) in DCM (5 mL) were added(S)-10-((dimethylamino)methyl)-4-ethyl-4,9-dihydroxy-1,12-dihydro-14H-pyrano[3′,4′:6,7]indolizino [1,2-b]quinoline-3,14(4H)-dione (SM-6, 350 mg, 0.9 mmol), DIPEA (0.42 mL, 2.7 mmol) and DMAP (10 mg, 0.09 mmol) at 0° C. The resultant reaction mixture was stirred RT for 16 h.

Progress of the reaction was monitored by TLC. After complete consumption of starting materials, the reaction mixture was diluted with water (100 mL) and extracted with DCM (2×10 mL). The combined organic layer was washed with water (10 mL) and brine solution (10 mL), dried over anhydrous Na2SO4, filtered and concentrated under reduced pressure to obtain crude compound, which was purified by Prep.HPLC to obtain the title compound (34 mg, 4.1%) as an off white solid.

¹H NMR (400 MHz, DMSO-d₆): δ ppm 8.95 (s, 1H), 8.01-8.22 (m, 4H), 7.61-7.74 (m, 2H), 7.34 (s, 1H), 7.07-7.16 (m, 2H), 6.62 (d, J=8.93 Hz, 2H), 6.52 (d, J=1.22 Hz, 1H), 5.43 (s, 2H), 5.27-5.35 (m, 2H), 3.67-3.82 (m, 6H), 3.46-3.55 (m, 4H), 2.95 (t, J=8.68 Hz, 2H), 2.55-2.61 (m, 3H), 2.32-2.38 (m, 2H), 2.21 (s, 6H), 1.88 (tt, J=13.83, 7.02 Hz, 3H), 1.61-1.72 (m, 2H), 1.46 (s, 6H), 0.89 (t, J=7.34 Hz, 3H); ESI MS m/z calcd. For C₅₂H₅₄ClN₉O₇S ([M+H]+) 984.36; found 984.4.

### Example S59. Preparation of (8S,11R,13S,14S,17R)-17-acetyl-11-(4-((6-(((S)-1-(((S)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1-oxo-5-ureidopentan-2-yl)amino)-3-methyl-1-oxobutan-2-yl)amino)hexyl)(methyl)amino)phenyl)-13-methyl-3-oxo-(Compound No. 59)

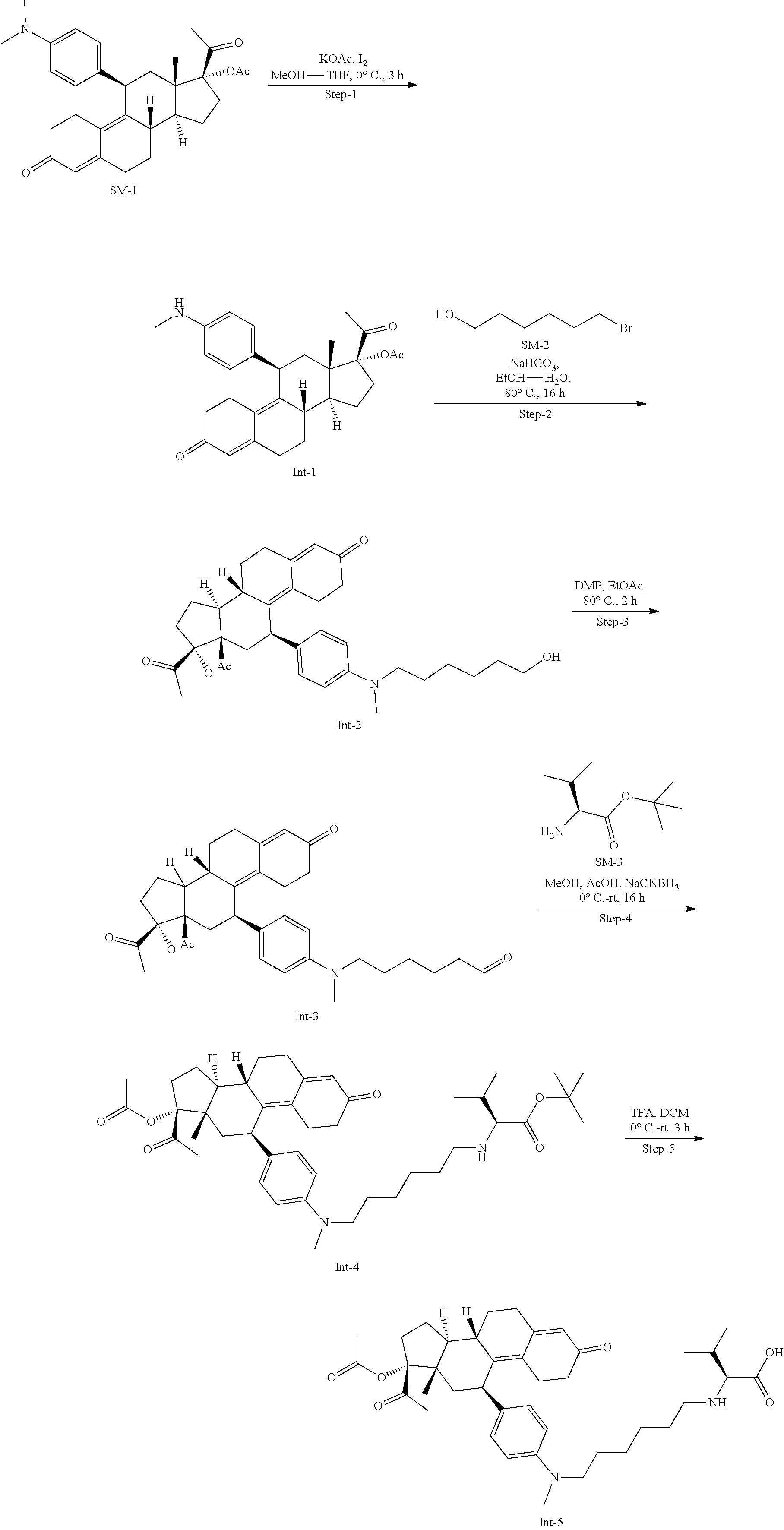

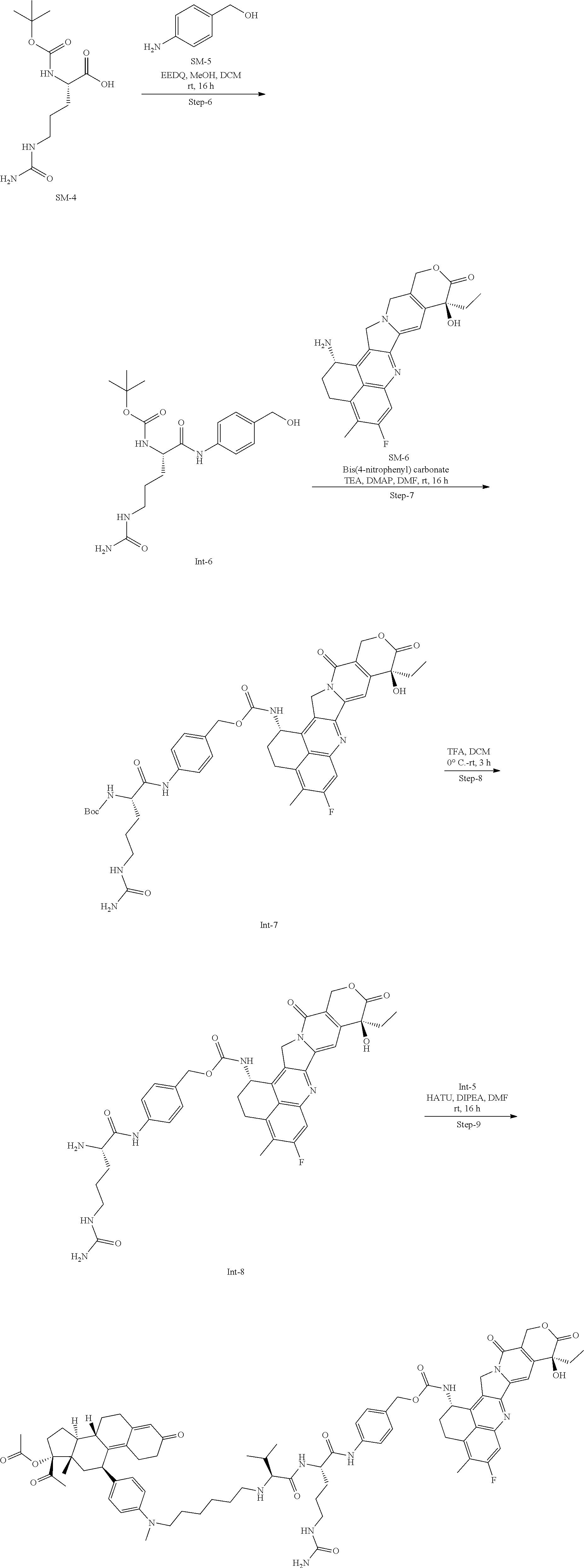

### Step-1: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-1, 10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The resultant reaction mixture was allowed to room temperature and stirred for 3 h. Progress of the reaction was monitored by TLC. After complete consumption of starting materials, the reaction mixture was quenched with sodium thiosulfate (Na₂S₂O₃) solution (50 g in 30 mL water) and extracted with ethyl acetate (2×200 mL). The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 8.0 g, 82%) as an off-white solid which was used in next step without any further purification.

### Step-2: Synthesis of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2)

To a stirred solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 4 g, 8.67 mmol, 1.0 eq) and 6-bromohexan-1-ol (SM-2, 7.81 g, 43.38 mmol, 5 eq) in ethanol (40 mL) and water (40 mL) was added NaHCO₃ (7.37 g, 86.76 mmol, 10 eq) at room temperature. The resultant reaction mixture was heated to 80° C. and stirred for 16 h. Progress of the reaction was monitored by TLC. After complete consumption of starting materials, the reaction mixture was filtered through a pad of celite bed and washed with ethyl acetate (40 mL). Filtrate was concentrated under reduced pressure, diluted with water (120 mL) and extracted with ethyl acetate (2×200 mL), The combined organic extracts were washed with brine (100 mL) and dried over anhydrous sodium sulphate, filtered and concentrated under reduced pressure to obtain crude compound. The crude compound was purified by combi-flash chromatography, by eluting with 70% ethyl acetate in heptane to afford (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 2.6 g, 53%) as an off-white solid.

### Step-3: Synthesis of (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-3)

To a stirred solution of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 500 mg, 0.891 mmol, 1 eq) in ethyl acetate (40 mL), was added dess-martin periodinane (DMP, 1.1 g, 2.67 mmol, 3 eq) portion wise at 0° C. The resultant reaction mixture was heated to 80° C. and stirred for 2 h. Progress of the reaction was monitored by TLC. After complete consumption of starting materials, the reaction mixture was quenched with 50% aqueous Na₂S₂O₃ solution (10 mL), sat. NaHCO₃ solution (15 mL) and extracted with ethyl acetate (2×25 mL). The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-3, 450 mg, 92%) as a brown solid.

### Step-4: Synthesis of tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valinate (Int-4)

To a stirred solution of (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-3, 1 g, 1.78 mmol, 1 eq) in Methanol (20 mL), tert-butyl L-valinate (309 mg, 1.78 mmol, 1 eq) and acetic acid (0.5 ml) were added at 0° C. The resultant reaction mixture was stirred at room temperature for 2 h. After that sodium cyanoborohydride (236 mg, 3.57 mmol, 2.0 eq) was added to the above reaction mixture at 0° C. and the resultant reaction mixture was stirred at room temperature for 16 h. Progress of the reaction was monitored by TLC. After complete consumption of starting materials, the reaction mixture was cooled to 0° C. and quenched with sat. NaHCO₃ solution (50 mL), extracted with DCM (2×100 mL). The combined organic layer was washed with water (50 mL) and brine (50 mL), dried over anhydrous sodium sulphate and concentrated under reduced pressure to afford tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valinate (Int-4, 1.0 g, 78%) as an orange solid.

### Step-5: Synthesis of (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valine (Int-5)

To a stirred solution of tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valinate (Int-4, 1 g, 1.39 mmol, 1 eq) in dichloromethane (10 mL) was added trifluoro acetic acid (10 mL) at 0° C. and the resultant reaction mixture was allowed to stir at room temperature for 3 h. Progress of the reaction was monitored by TLC. After complete consumption of starting material, the reaction mixture was evaporated under reduced pressure and washed with diethyl ether, dried under vacuum to afford (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valine (Int-5, 500 mg, 58%) as an orange solid.

### Step-6: Synthesis of tert-butyl (S)-(1-((4-(hydroxymethyl)phenyl)amino)-1-oxo-5-ureidopentan-2-yl)carbamate (Int-6)

To a stirred solution of ((S)-2-((tert-butoxycarbonyl)amino)-5-ureidopentanoic acid (SM-4, 1 g, 3.60 mmol, 1.0 eq) in Methanol (10 mL) and DCM (10 mL) (4-aminophenyl) methanol (450 mg, 3.60 mmol, 1 eq) and N-Ethoxycarbonyl-2-ethoxy-1,2-dihydroquinoline (EEDQ, 1.8 g, 7.20 mmol, 2 eq) were added at room temperature. The resultant reaction mixture was stirred at room temperature for 16 h. Progress of the reaction was monitored by TLC. After complete consumption of starting material, the reaction mixture was concentrated under reduced pressure to obtain crude compound, which was purified by combi-flash chromatography by eluting with 0-70% ethyl acetate in heptane to afford tert-butyl (S)-(1-((4-(hydroxymethyl)phenyl)amino)-1-oxo-5-ureidopentan-2-yl)carbamate (Int-6, 500 mg, 36%) as an off-white solid.

### Step-7: Synthesis of 4-((S)-2-((tert-butoxycarbonyl)amino)-5-ureidopentanamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Int-7)

To a stirred solution of tert-butyl (S)-(1-((4-(hydroxymethyl)phenyl)amino)-1-oxo-5-ureidopentan-2-yl)carbamate (Int-6, 750 mg, 1.97 mmol, 1.0 eq) in DMF (5 mL). Triethyl amine (0.68 ml, 2.95 mmol, 2.5 eq), Bis (4-nitrophenyl)carbonate (908 mg, 2.95 mmol, 1.5 eq) and DMAP (48 mg, 0.39 mmol, 0.2 eq) were added at room temperature. The resultant reaction mixture was stirred at room temperature for 3 h. After that (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (SM-6, 858 mg, 1.97 mmol, 1 eq) was added and stirred at room temperature for 16 h. Progress of the reaction was monitored by TLC. After complete consumption of starting materials, the reaction mixture was diluted with water (50 ml) and extracted with DCM (2×50 mL). The combined organic layer was washed with water (50 mL) and brine (50 mL), dried over anhydrous sodium sulphate and concentrated under reduced pressure to obtain crude compound, which was purified by combi-flash chromatography (by eluting with 0-80% ethyl acetate in heptane) to afford Int-7 (250 mg, 15%) as a pale brown solid.

### Step-8: Synthesis of 4-((S)-2-amino-5-ureidopentanamido)benzyl ((1S,9S)-9-ethyl-5-fluoro-9-hydroxy-4-methyl-10,13-dioxo-2,3,9,10,13,15-hexahydro-1H,12H-benzo[de] pyrano[3′,4′:6,7]indolizino[1,2-b]quinolin-1-yl)carbamate (Int-8)

To a stirred solution of 4-((S)-2-((tert-butoxycarbonyl)amino)-5-ureidopentanamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]305uinoline-1-yl)carbamate (Int-7, 350 mg, 0.41 mmol, 1 eq) in dichloromethane (5 mL) was added trifluoro acetic acid (5 mL) at 0° C. and the resultant reaction mixture was allowed to stir at room temperature for 3 h. Progress of the reaction was monitored by TLC. After complete consumption of starting material, the reaction mixture was evaporated under reduced pressure, washed with diethyl ether and dried under vacuum to afford 4-((S)-2-amino-5-ureidopentanamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Int-9, 300 mg, 97%) as a pale yellow solid.

### Step-9: Synthesis of (8S,11R,13S,14S,17R)-17-acetyl-11-(4-((6-(((S)-1-(((S)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1-oxo-5-ureidopentan-2-yl)amino)-3-methyl-1-oxobutan-2-yl)amino)hexyl)(methyl)amino)phenyl)-13-methyl-3-oxo-2,3,6,7,8,11,12,13,14,15,16,17-dodecahydro-1H-cyclopenta[a]phenanthren-17-yl acetate

To a stirred solution (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl) (methyl)amino)hexyl)-L-valine (Int-5, 120 mg, 0.18 mmol) in DMF (1 mL) were added 4-((S)-2-amino-5-ureidopentanamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Int-9, 134 mg, 0.18 mmol, 1 eq) and N,N-Diisopropylethylamine (71 mg, 0.54 mmol, 3.0 eq) at room temperature and stirred for 5 min. After that HATU (106 mg, 0.27 mmol, 1.5 eq) was added at room temperature. The resultant reaction mixture was stirred at room temperature for 16 h. Progress of the reaction was monitored by TLC. After complete consumption of starting material, the reaction mixture was diluted with ice cold water (50 mL) and extracted with DCM (2×50 mL), The combined organic layer was washed with brine solution (50 mL), dried over anhydrous sodium sulphate, filtered, and concentrated under reduced pressure to obtain crude compound, which was purified by prep HPLC to afford (8S,11R,13S,14S,17R)-17-acetyl-11-(4-((6-(((S)-1-(((S)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1-oxo-5-ureidopentan-2-yl)amino)-3-methyl-1-oxobutan-2-yl)amino)hexyl)(methyl)amino)phenyl)-13-methyl-3-oxo-2,3,6,7,8,11,12,13,14,15,16,17-dodecahydro-1H-cyclopenta[a]phenanthren-17-yl acetate (20 mg, 8%) as an off white solid.

¹H NMR (400 MHz, DMSO-d₆) δ ppm 10.20 (s, 1H), 8.59-8.89 (m, 2H), 8.47 (br dd, J=9.44, 5.32 Hz, 1H), 8.05 (br d, J=8.88 Hz, 1H), 7.78 (d, J=10.88 Hz, 1H), 7.60 (br d, J=8.50 Hz, 2H), 7.37 (br d, J=8.38 Hz, 2H), 7.32 (s, 1H), 6.98 (br d, J=8.63 Hz, 2H), 6.59 (br d, J=8.88 Hz, 2H), 6.51 (s, 1H), 5.96-6.04 (m, 1H), 5.67 (s, 1H), 5.44 (br s, 3H), 5.29 (br d, J=4.75 Hz, 3H), 5.09 (s, 2H), 4.49-4.69 (m, 1H), 4.39 (br d, J=6.88 Hz, 1H), 3.71 (br d, J=5.75 Hz, 1H), 3.22 (br d, J=7.13 Hz, 2H), 3.05-3.16 (m, 2H), 2.91-3.03 (m, 2H), 2.70-2.89 (m, 6H), 2.58-2.69 (m, 2H), 2.54 (br s, 3H), 2.45-2.48 (m, 5H), 2.38 (s, 2H), 2.24-2.36 (m, 3H), 2.14 (br d, J=14.38 Hz, 5H), 2.06-2.11 (m, 4H), 1.97-2.02 (m, 3H), 1.79-1.96 (m, 4H), 1.50-1.77 (m, 7H), 1.37-1.49 (m, 4H), 1.19-1.36 (m, 7H), 1.03 (br d, J=6.75 Hz, 3H), 0.93 (br d, J=6.75 Hz, 3H), 0.88 (br t. J=7.32 Hz, 3H), 0.23 (s, 3H); LCMS: 96.26%; ESI MS m/z calcd. For C₇₈H₉₄FN₉O₁₃ ([M+H]⁺) 1384.6; found 693.7 HPLC: 95.54%.

### Example S60. Preparation of (8S,11R,13S,14S,17R)-17-acetyl-11-(4-((6-(((S)-1-(((S)-1-((4-((((((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)oxy)carbonyl) (methyl)amino)methyl)phenyl)amino)-1-oxo-5-ureidopentan-2-yl)amino)-3-methyl-1-oxobutan-2-yl)amino)hexyl)(methyl)amino)phenyl)-13-methyl-3-oxo-(Compound No. 60)

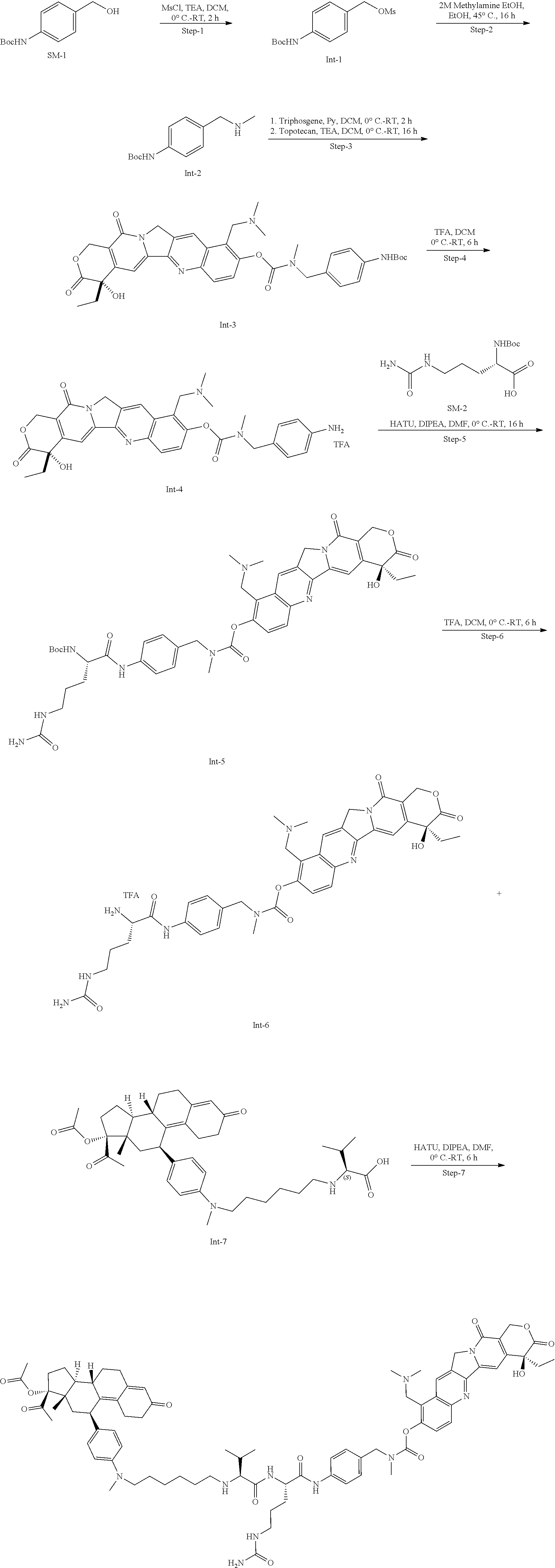

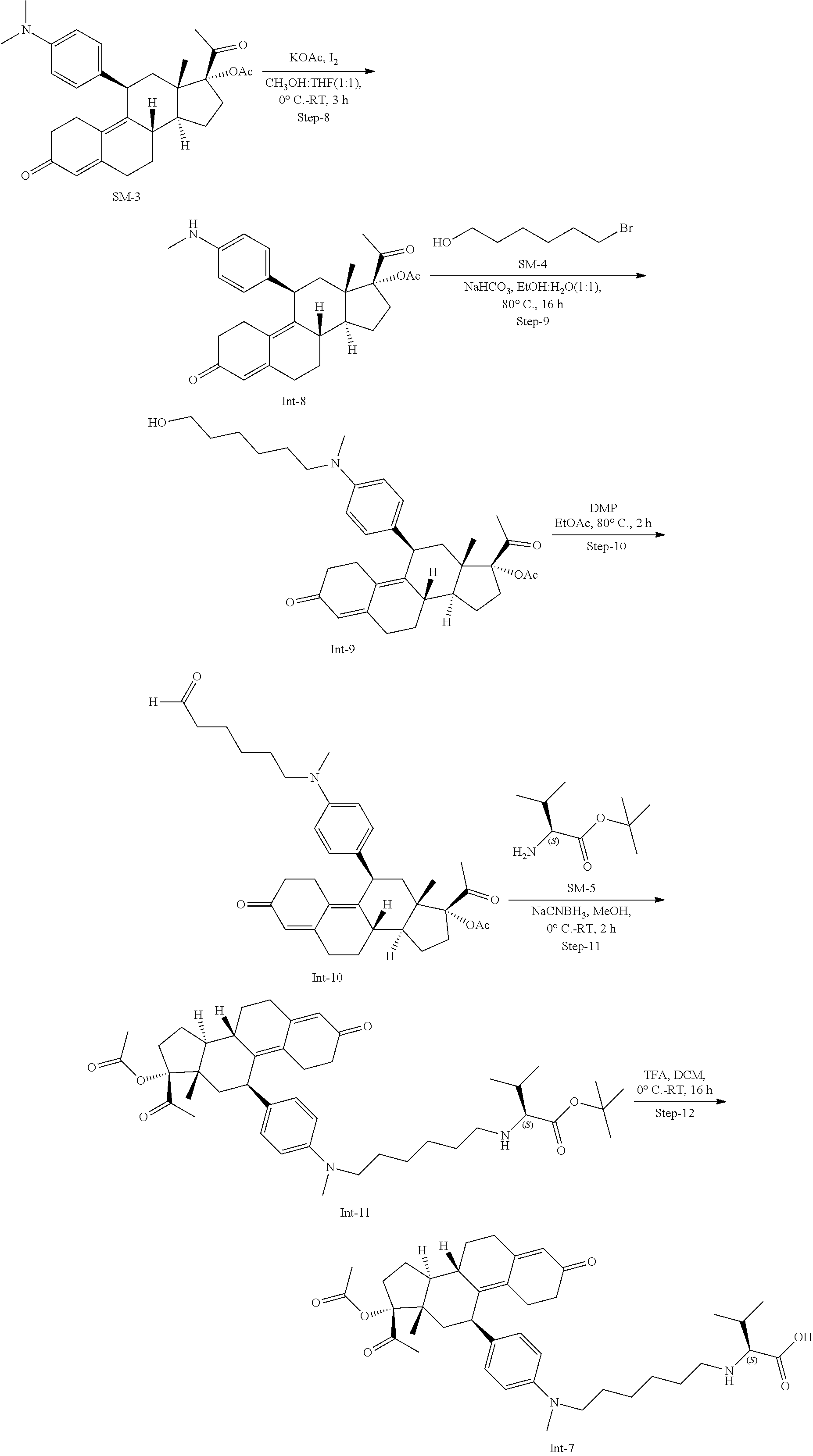

### Step-1: Synthesis of 4-((tert-butoxycarbonyl)amino)benzyl methanesulfonate (Int-1)

To a stirred solution of tert-butyl (4-(hydroxymethyl)phenyl)carbamate (SM-1, 5 g, 22.3 mmol, 1.0 eq) in DCM (50 mL) were added TEA (9.4 mL, 67.1 mmol, 3 eq) and Methane sulfonyl chloride (3.5 mL, 44.7 mmol, 2 eq) drop wise at 0° C. The resultant reaction mixture was allowed to room temperature and stirred for 2 h. Progress of the reaction was monitored by TLC. After complete consumption of starting materials, the reaction mixture was poured into ice cold water (100 mL) and extracted with DCM (2×100 mL). The combined organic extracts were washed with brine (100 mL) and dried over anhydrous sodium sulphate, filtered, and concentrated under reduced pressure to afford 4-((tert-butoxycarbonyl)amino)benzyl methane sulfonate (Int-1, 3 g, 44%) as a brown solid.

### Step-2: Synthesis of tert-butyl (4-((methylamino)methyl)phenyl)carbamate (Int-2)

To a stirred solution of 4-((tert-butoxycarbonyl)amino)benzyl methane sulfonate (Int-1, 2.5 g, 8.3 mmol, 1.0 eq) in EtOH (30 mL) were added 2M Methylamine in EtOH (20.7 mL, 41.5 mmol, 5 eq) at 0° C. The resultant reaction mixture was allowed to stir at 45° C. for 16 h. Progress of the reaction was monitored by TLC. After complete consumption of starting materials, the volatiles were evaporated under reduced pressure to obtain crude compound, which was purified by combi-flash by eluting with 5-10% MeOH in DCM to afford tert-butyl (4-((methylamino)methyl)phenyl)carbamate (Int-2, 800 mg, 41%) as an off white solid.

### Step-3: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-((tert-butoxycarbonyl)amino)benzyl)(methyl)carbamate (Int-3)

To a stirred solution of tert-butyl (4-((methylamino)methyl)phenyl)carbamate (Int-2, 2 g, 5 g, 8.46 mmol, 1.0 eq) in DCM (40 mL) were added pyridine (1.7 mL, 21.1 mmol, 2.5 eq) and Triphosgene (1.2 g, 4.23 mmol, 0.5 eq) solution in DCM (20 mL) over a period of 1 h at 0° C. The resultant reaction mixture was allowed to room temperature and stirred for 1 h. Progress of the reaction was monitored by TLC (non-polar spot was observed). After complete consumption of starting materials, the reaction mixture was poured into ice cold water (80 mL) and extracted with DCM (2×60 mL). The combined organic extracts were washed with brine (100 mL) and dried over anhydrous sodium sulphate, filtered, and concentrated under reduced pressure to afford tert-butyl (4-(((chlorocarbonyl)(methyl)amino)methyl)phenyl)carbamate (2 g, crude) as a brown solid.

To a solution of (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione·hydrochloride (2 g, 4.74 mmol, 1 eq) in DCM (40 mL) was added DIPEA (4.15 mL, 23.7 mmol, 5 eq) and DMAP (146 mg, 1.18 mmol, 0.25 eq) at 0° C. To this mixture above crude DCM solution was added. The reaction mixture was allowed to room temperature and stirred for 16 h. Progress of the reaction was monitored by TLC. After complete consumption of starting materials, the reaction mixture was quenched with water (100 mL) and extracted with ethyl acetate (2×100 mL). The combined organic extracts were washed with brine (100 mL) and dried over anhydrous sodium sulphate, filtered, and concentrated under reduced pressure to obtain crude compound, which was purified by combi-flash column chromatography by eluting with 5-10% MeOH in DCM to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-((tert-butoxycarbonyl) amino)benzyl)(methyl)carbamate (Int-3, 2 g, 61%) as a brown solid.

### Step-4: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-aminobenzyl)(methyl)carbamate·TFA (Int-4)

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-((tert-butoxycarbonyl) amino)benzyl)(methyl)carbamate (Int-3, 2 g, 2.9 mmol, 1.0 eq) in DCM (40 mL) was added TFA (2.2 mL, 2.92 mmol, 10.0 eq) at 0° C. under nitrogen atmosphere. The resultant reaction mixture was allowed to room temperature and stirred for 6 h. Progress of the reaction was monitored by TLC. After complete consumption of starting material, the volatiles were evaporated under reduced pressure to obtain crude compound, which was washed with diethyl ether (20 mL) and dried under vacuum to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-aminobenzyl)(methyl)carbamate·TFA (Int-4, 3 g. Crude) as a brown solid.

### Step-5: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-((S)-2-((tert-butoxycarbonyl)amino)-5-ureidopentanamido)benzyl)(methyl)carbamate (Int-5)

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-aminobenzyl) (methyl)carbamate·TFA (Int-4, 3 g, 5.14 mmol, 1 eq) in DMF (30 mL) were added DIPEA (4.4 mL, 3.32 mmol, 5 eq), HATU (2.9 g, 7.71 mmol, 1.5 eq) and (S)-2-((tert-butoxycarbonyl)amino)-5-ureidopentanoic acid (SM-2, 2.8 g, 10.2 mmol, 2.0 eq) at room temperature. The resultant reaction mixture was stirred at room temperature for 16 h. Progress of the reaction was monitored by TLC. After complete consumption of starting materials, the reaction mixture was quenched with water (100 mL) and extracted with 10% MeOH in DCM (2×100 mL). The combined organic extracts were washed with brine (100 mL) and dried over anhydrous sodium sulphate, filtered, and concentrated under reduced pressure to obtain crude compound, which was washed with diethyl ether (20 mL) and dried under vacuum to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-((S)-2-((tert-butoxycarbonyl)amino)-5-ureidopentanamido)benzyl)(methyl)carbamate (Int-5, 2.5 g, 58%) as an off white solid.

### Step-6: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-((S)-2-amino-5-ureidopentanamido)benzyl)(methyl)carbamate·TFA (Int-6)

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-((S)-2-((tert-butoxycarbonyl)amino)-5-ureidopentanamido)benzyl)(methyl)carbamate (Int-5, 2 g, 2.3 mmol, 1.0 eq) in DCM (20 mL) was added TFA (1.85 mL, 23.78 mmol, 10.0 eq) at 0° C. under nitrogen atmosphere. The resultant reaction mixture was allowed to room temperature and stirred for 6 h. Progress of the reaction was monitored by TLC. After complete consumption of starting materials, the volatiles were evaporated under reduced pressure to obtain crude compound, which was washed with diethyl ether (20 mL) and dried under vacuum to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (4-((S)-2-amino-5-ureidopentanamido)benzyl)(methyl)carbamate·TFA (Int-6, 1.3 g, 74%) as a brown solid.

### Step-7: Synthesis of (8S,11R,13S,14S,17R)-17-acetyl-11-(4-((6-(((S)-1-(((S)-1-((4-((((((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)oxy)carbonyl) (methyl)amino)methyl)phenyl)amino)-1-oxo-5-ureidopentan-2-yl)amino)-3-methyl-1-oxobutan-2-yl)amino)hexyl)(methyl)amino)phenyl)-13-methyl-3-oxo-2,3,6,7,8,11,12,13,14,15,16,17-dodecahydro-1H-cyclopenta[a]phenanthren-17-yl acetate

To a stirred solution of (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl) (methyl)amino)hexyl)-L-valine (Int-7, 500 mg, 0.69 mmol, 1 eq) in DMF (10 mL) were added DIPEA (0.39 mL, 2.27 mmol, 5 eq), HATU (258 mg, 0.68 mmol, 1.5 eq) and (S)-10-((dimethylamino)methyl)-4-ethyl-4-hydroxy-3,14-dioxo-3,4,12,14-tetrahydro-1H-pyrano [3′,4′:6,7]indolizino[1,2-b]quinolin-9-yl (4-((S)-2-amino-5-ureidopentanamido)benzyl) (methyl)carbamate·TFA (Int-6, 336 mg, 0.45 mmol, 1.0 eq) at room temperature. The resultant reaction mixture was stirred at room temperature for 16 h. Progress of the reaction was monitored by TLC. After complete consumption of starting materials, the reaction mixture was poured into ice cold water (50 ml). The obtained solids were filtered and washed with water, dried under vacuum. The crude solid compound was purified by Prep.HPLC, pure fractions were lyophilised to obtain the title compound (29 mg, 4%) as an off white solid.

¹H NMR (400 MHz, DMSO-d₆): δ ppm 10.20-10.27 (m, 1H), 9.76-9.92 (m, 1H), 9.10 (d, J=3.30 Hz, 1H), 8.84 (d, J=7.70 Hz, 1H), 8.69-8.79 (m, 1H), 8.47-8.57 (m, 1H), 8.31-8.37 (m, 1H), 7.77-7.89 (m, 1H), 7.61-7.70 (m, 2H), 7.32-7.42 (m, 3H), 6.95-7.24 (m, 2H), 6.59-6.71 (m, 2H), 6.01-6.11 (m, 1H), 5.68 (s, 1H), 5.45 (s, 2H), 5.34 (s, 3H), 4.76-4.87 (m, 3H), 4.58 (dd, J=13.20, 7.95 Hz, 2H), 4.53 (s, 1H), 4.41 (d, J=6.60 Hz, 2H), 3.73 (s, 1H), 3.25 (t, J=7.34 Hz, 2H), 3.14 (s, 2H), 3.03-3.09 (m, 1H), 2.96-3.02 (m, 3H), 2.92 (s, 3H), 2.85 (s, 3H), 2.78 (s, 6H), 2.57 (s, 1H), 2.32-2.43 (m, 2H), 2.20-2.24 (m, 2H), 2.13-2.18 (m, 2H), 2.10 (s, 4H), 2.00 (s, 3H), 1.84-1.93 (m, 3H), 1.57-1.78 (m, 7H), 1.36-1.52 (m, 6H), 1.22-1.33 (m, 6H), 1.04 (d, J=6.85 Hz, 3H), 0.86-0.96 (m, 6H); ESI MS m/z calcd. For C₇₈H₉₈N₁₀O₁₃ ([M+H]⁺) 1383.70; found 1384.06.

### Step-8: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-8)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-3, 10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The resultant reaction mixture was allowed to room temperature and stirred for 3 h. Progress of the reaction was monitored by TLC. After complete consumption of starting materials, the reaction mixture was quenched with sodium thiosulfate (Na₂S₂O₃) solution (50 g in 30 mL water) and extracted with ethyl acetate (2×200 mL), The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methylamino)phenyl)-3-oxo-2,3,6,7,8,11,12,13,14,15,16, 17-dodecahydro-1H-cyclopenta[a]phenanthren-17-yl acetate (Int-8, 8.0 g, 82%) as an off-white solid.

Note: proceeded to next step without any further purification.

### Step-9: Synthesis of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthrene-17-yl acetate (Int-9)

To a solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-8, 4 g, 8.67 mmol, 1.0 eq) and 6-bromohexan-1-ol (SM-4, 7.81 g, 43.38 mmol, 5 eq) in ethanol (40 mL) and water (40 mL) was added NaHCO₃ (7.37 g, 86.76 mmol, 10 eq) at room temperature. The resultant reaction mixture was heated to 80° C. and stirred for 16 h. Progress of the reaction was monitored by TLC. After complete consumption of starting materials, the reaction mixture was filtered through a pad of celite bed and washed with ethyl acetate (40 mL). The obtained filtrate was concentrated under reduced pressure and diluted with water (120 mL) and extracted with ethyl acetate (2×200 mL). The combined organic extracts were washed with brine (100 mL) and dried over anhydrous sodium sulphate, filtered, and concentrated under reduced pressure to obtain crude compound, which was purified by combi-flash chromatography by eluting with 70% ethyl acetate in heptane to afford (8S,11R,13S,14S,17R)-17-acetyl-11-(4-((6-hydroxyhexyl)(methyl)amino) phenyl)-13-methyl-3-oxo-2,3,6,7,8,11,12,13,14,15,16,17-dodecahydro-1H-cyclopenta[a]phenanthrene-17-yl acetate (Int-9, 2.6 g, 53%) as an off-white solid.

### Step-10: Synthesis of (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-10)

To a stirred solution of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthrene-17-yl acetate (Int-9, 500 mg, 0.891 mmol, 1 eq) in ethyl acetate (40 mL), was added Dess-Martin periodinane (DMP, 1.1 g, 2.67 mmol, 3 eq), portion wise at 0° C. The resultant reaction mixture was heated to 80° C. and stirred for 2 h. Progress of the reaction was monitored by TLC. After complete consumption of starting materials, the reaction mixture was quenched with 50% aqueous Na₂S₂O₃ solution (10 mL), sat. NaHCO₃ solution (15 mL) and extracted with ethyl acetate (2×25 mL). The combined organic extracts were washed with brine (100 mL) and dried over anhydrous sodium sulphate, filtered, and concentrated under reduced pressure to afford (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-10, 450 mg, 92%) as a brown solid.

### Step-11: Synthesis of tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valinate (Int-11)

To a stirred solution of tert-butyl L-valinate (SM-5, 1.23 g, 7.15 mmol) in MeOH (20 mL) were added (8S,11R,13S,14S,17R)-17-acetyl-13-methyl-11-(4-(methyl(6-oxohexyl)amino)phenyl)-3-oxo-2,3,6,7,8,11,12,13,14,15,16,17-dodecahydro-1H-cyclopenta[a]phenanthren-17-yl acetate (Int-10, 2 g, 3.57 mmol) and AcOH (0.2 mL) at 0° C. and warmed the reaction mixture to room temperature, stirred for 10 min. After that NaCNBH₃ (450 mg, 7.15 mmol) was added at 0° C. The resultant reaction mixture was stirred at room temperature for 2 h Progress of the reaction was monitored by TLC. After complete consumption of starting materials, the reaction mixture was cooled to 0° C. and quenched with sat. NaHCO₃ solution (100 mL), extracted with 10% MeOH in DCM (2×100 mL). The combined organic layer was washed with water (50 mL), brine (50 mL) and dried over anhydrous Na₂SO₄, filtered and concentrated under reduced pressure to obtain crude product, which was washed with diethyl ether (20 mL) and dried under vacuum to afford tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valinate (Int-11, 1.2 g, 6.4%) as a brown solid.

### Step-12: Synthesis of (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valine (Int-7)

To a stirred solution of tert-butyl (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl) (methyl)amino)hexyl)-L-valinate (Int-11, 1.2 g, 1.67 mmol, 1.0 eq) in DCM (12 mL) under nitrogen atmosphere was added TFA (12 mL, 10 vol) at 0° C. The resultant reaction mixture was warmed to room temperature and stirred for 16 h. Progress of the reaction was monitored by TLC. After complete consumption of starting materials, the volatiles were evaporated under reduced pressure to obtain crude compound, which was washed with diethyl ether (20 mL) and dried under vacuum to afford (6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(methyl)amino)hexyl)-L-valine (Int-7, 900 mg, crude) as a brown solid.

### Example S61. Preparation of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)amino)hexyl)piperazine-1-carboxylate (Compound No. 61)

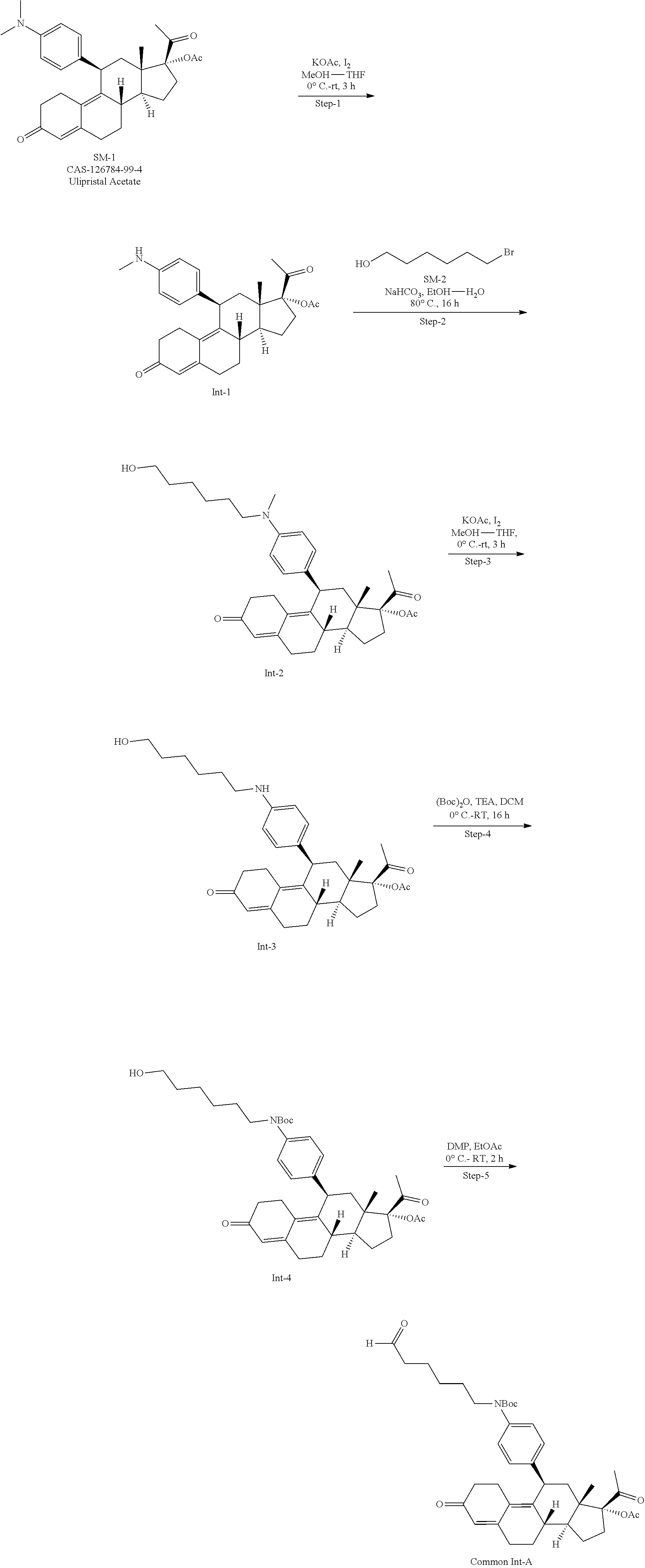

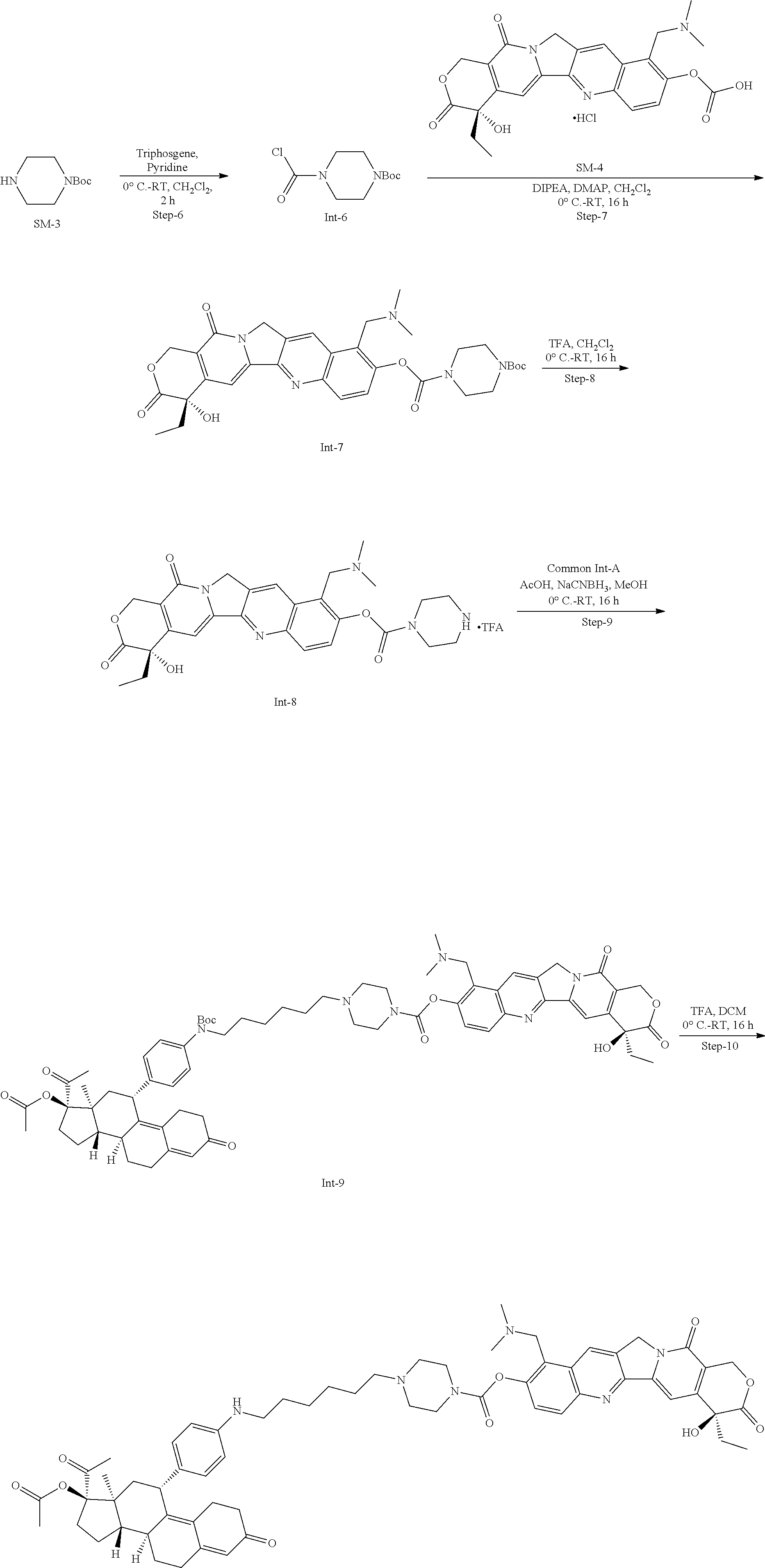

### Step-1: Synthesis of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1)

To a stirred solution of (8S,11R,13S,14S,17R)-17-acetyl-11-(4-(dimethylamino)phenyl)-13-methyl-3-oxo-2,3,6,7,8,11,12,13,14,15,16,17-dodecahydro-1H-cyclopenta[a]phenanthren-17-yl acetate (SM-1, 10 g, 21 mmol, 1.0 eq) in methanol (150 mL) and THF (150 mL) were added KOAc (20.6 g, 210 mmol, 10 eq) and Iodine (13.1 g, 105 mmol, 5 eq) at 0° C. The resultant reaction mixture was allowed to stir at room temperature for 3 h. Progress of the reaction was monitored by TLC. After complete consumption of SMs, the reaction mixture was quenched with sodium thiosulfate (Na₂S₂O₃) solution (50 g in 30 mL water) and extracted with ethyl acetate (2×200 mL). The combined organic extracts were washed with brine (100 mL) and dried over anhydrous sodium sulphate, filtered and concentrated under reduced pressure to afford (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 8.0 g, 82%) as an off-white solid, which was used in next step without any further purification.

### Step-2: Synthesis of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2)

To a solution of (8S,11R,13S,14S,17R)-17-acetyl-13-methyl-11-(4-(methylamino)phenyl)-3-oxo-2,3,6,7,8,11,12,13,14,15,16,17-dodecahydro-1H-cyclopenta[a]phenanthren-17-yl acetate (Int-1, 4 g, 8.67 mmol, 1.0 eq) and 6-bromohexan-1-ol (SM-2, 7.81 g, 43.38 mmol, 5 eq) in ethanol (40 mL) and water (40 mL) was added NaHCO₃ (7.37 g, 86.76 mmol, 10 eq) at room temperature. The resultant reaction mixture was heated to 80° C. and stirred for 16 h. Progress of the reaction was monitored by TLC. After complete consumption of starting materials, the reaction mixture was filtered through a pad of celite bed and washed with ethyl acetate (40 mL). The obtained filtrate was concentrated under reduced pressure and diluted with water (120 mL), extracted with ethyl acetate (2×200 mL). The combined organic extracts were washed with brine (100 mL), dried over anhydrous sodium sulphate, filtered and concentrated under reduced pressure to obtain crude compound, which was purified by combiflash chromatography (by eluting with 70% ethyl acetate in heptane) to afford (Int-2, 2.6 g, 53%) as an off-white solid.

### Step-3: [(8S,11R,13S,14S,17R)-17-acetyl-11-[4-(6-hydroxyhexylamino)phenyl]-13-methyl-3-oxo-1,2,6,7,8,11,12,14,15,16-decahydrocyclopenta[a]phenanthren-17-yl]acetate (Int-3)

To a stirred solution of (8S,11R,13S,14S,17R)-17-acetyl-11-(4-((6-hydroxyhexyl)(methyl)amino)phenyl)-13-methyl-3-oxo-2,3,6,7,8,11,12,13,14,15,16,17-dodecahydro-1H-cyclopenta[a]phenanthren-17-yl acetate (Int-2, 1.0 g, 1.8 mmol, 1 eq) in tetrahydrofuran (15 mL) and methanol (15 mL) were added potassium acetate (1.74 g, 18 mmol, 10 eq) and iodine (2.25 g, 8.9 mmol, 5 eq) at 0° C. The resultant reaction mixture was allowed to stir at 0° C. for 3 h. Progress of the reaction was monitored by LCMS. After complete consumption of starting materials, the reaction mixture was quenched with sodium thio sulphate solution (50 mL) and extracted with DCM (2×50 mL). The combined organic layer was washed with water (50 mL), brine solution (50 mL), and dried over anhydrous sodium sulphate, filtered, and concentrated under reduced pressure to obtain crude compound, which was purified by combi flash chromatography (by eluting with 65% ethyl acetate in n-heptane) to afford [(8S,11R,13S,14S,17R)-17-acetyl-11-[4-(6-hydroxyhexylamino)phenyl]-13-methyl-3-oxo-1,2,6,7,8,11,12,14,15,16-decahydrocyclopenta[a]phenanthren-17-yl]acetate (Int-3, 0.5 g, 50% yield) as an off-white solid.

### Step-4: Synthesis of (8S,11R,13S,14S,17R)-17-acetyl-11-(4-((tert-butoxycarbonyl) (6-hydroxyhexyl)amino)phenyl)-13-methyl-3-oxo-2,3,6,7,8,11,12,13,14,15,16,17-dodecahydro-1H-cyclopenta[a]phenanthren-17-yl acetate (Int-4)

To a stirred solution of [(8S,11R,13S,14S,17R)-17-acetyl-11-[4-(6-hydroxyhexylamino)phenyl]-13-methyl-3-oxo-1,2,6,7,8,11,12,14,15,16-decahydrocyclopenta[a]phenanthren-17-yl]acetate (Int-3, 3.5 g, 6.4 mmol, 1 eq) in dichloromethane (70 mL) was added di-tert-butyl dicarbonate (1.67 g, 7.7 mmol, 1.2 eq) and followed by the addition of triethylamine (2.67 mL, 19 mmol, 3 eq) at 0° C. The resultant reaction mixture was warmed to room temperature and stirred for 16 h. Progress of the reaction was monitored by LCMS. After complete consumption of starting materials, the reaction mixture was diluted with water (50 mL) and extracted with DCM (2×50 mL). The combined organic layer was washed with water (50 mL), brine solution (50 mL), and dried over anhydrous sodium sulphate, filtered, and concentrated under reduced pressure to obtain crude compound, which was purified by combi flash chromatography by eluting with 65% ethyl acetate in heptane to afford (8S,11R,13S,14S,17R)-17-acetyl-11-(4-((tert-butoxycarbonyl) (6-hydroxyhexyl)amino)phenyl)-13-methyl-3-oxo-2,3,6,7,8,11,12,13,14,15,16,17-dodecahydro-1H-cyclopenta[a]phenanthren-17-yl acetate (Int-4, 2.0 g, 48% yield) as a brown colour liquid.

### Step-5: Synthesis of (8S,11R,13S,14S,17R)-17-acetyl-11-(4-((tert-butoxycarbonyl) (6-oxohexyl)amino)phenyl)-13-methyl-3-oxo-2,3,6,7,8,11,12,13,14,15,16,17-dodecahydro-1H-cyclopenta[a]phenanthren-17-yl acetate (Common Int-A)

To a stirred solution of (8S,11R,13S,14S,17R)-17-acetyl-11-(4-((tert-butoxycarbonyl) (6-hydroxyhexyl)amino)phenyl)-13-methyl-3-oxo-2,3,6,7,8,11,12,13,14,15,16,17-dodecahydro-1H-cyclopenta[a]phenanthren-17-yl acetate (Int-4, 2.0 g, 3.1 mmol, 1 eq) in EtOAc (40 mL) was added dess-martin periodinane (DMP, 2.62 g, 6.2 mmol, 2 eq) at 0° C. The resultant reaction mixture was warmed to room temperature and stirred for 2 h. Progress of the reaction was monitored by TLC. After complete consumption of SM, the reaction mixture was quenched with sodium bicarbonate solution (50 mL) and extracted with EtOAc (2×50 mL). The combined organic layers were washed with water (50 mL) then brine solution (50 mL), and dried over anhydrous Na₂SO₄ and concentrated under reduced pressure to afford (8S,11R,13S,14S,17R)-17-acetyl-11-(4-((tert-butoxycarbonyl) (6-oxohexyl)amino)phenyl)-13-methyl-3-oxo-2,3,6,7,8,11,12,13,14,15,16,17-dodecahydro-1H-cyclopenta[a]phenanthren-17-yl acetate (Common Int-A. 1.7 g, 85% yield) as a brown solid.

### Step-6: Synthesis of tert-butyl 4-(chlorocarbonyl)piperazine-1-carboxylate (Int-6)

To a stirred solution of tert-butyl piperazine-1-carboxylate (SM-3, 5 g, 26.8 mmol, 1.0 eq) in DCM (100 mL) were added pyridine (2.97 g, 37.6 mmol, 1.4 eq) and triphosgene (3.19 g, 10.7 mmol, 0.4 eq) at 0° C. The resultant reaction mixture was warmed to room temperature and stirred for 2 h. Progress of the reaction was monitored by TLC. After complete consumption of starting material, the reaction mixture was washed with water (100 mL) and extracted with DCM (2×100 mL). The combined organic extracts were again washed with water (200 mL), brine (200 mL) and dried over anhydrous sodium sulphate, filtered, and concentrated under reduced pressure to afford tert-butyl 4-(chlorocarbonyl)piperazine-1-carboxylate (Int-6, 6.0 g, 90%) as a colourless oil.

### Step-7: Synthesis of (S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) piperazine-1,4-dicarboxylate (Int-7)

To a stirred solution of (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione. HCl salt (SM-4, 10 g, 23.7 mmol, 1.0 eq) in DCM (250 mL) were added DIPEA (15.3 g, 118 mmol, 5 eq) and DMAP (724 mg, 5.9 mmol, 0.25 eq) followed by addition of tert-butyl 4-(chlorocarbonyl)piperazine-1-carboxylate (Int-6, 5.89 g, 23.7 mmol, 1 eq) in DCM (mL) drop wise over a period of 10 min at 0° C. The resultant reaction mixture was warmed to room temperature and stirred for 16 h. Progress of the reaction was monitored by TLC. After complete consumption of starting materials, the reaction mixture was diluted with water (100 mL) and extracted with DCM (3×100 mL). The combined organic extracts were again washed with water (200 mL), brine (200 mL) and dried over anhydrous sodium sulphate, filtered, and concentrated under reduced pressure to afford crude compound. Which was purified by combiflash column by eluting with 7% methanol in DCM to afford (Int-7, 10 g, 66%) as an off white solid.

### Step-8: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate. TFA salt (Int-8)

To a stirred solution of (S)-1-(tert-butyl) 4-(10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)piperazine-1,4-dicarboxylate (Int-7, 1 g, 15 mmol, 1.0 eq) in DCM (20 mL) under nitrogen atmosphere was added TFA (3 mL) at 0° C. The resultant reaction mixture was warmed to room temperature and stirred for 16 h. Progress of the reaction was monitored by TLC. After complete consumption of starting materials, the volatiles were evaporated under reduced pressure to obtain crude material, which was diluted with sat. NaHCO₃ (50 mL) solution and extracted with EtOAc (2×100 mL). The combined organic layer was evaporated under reduced pressure to afford(S)-10-((dimethylamino)methyl)-4-ethyl-4-hydroxy-3,14-dioxo-3,4,12,14-tetrahydro-1H-pyrano[3′,4′:6,7]indolizine[1,2-b]quinolin-9-yl piperazine-1-carboxylate. TFA salt (Int-8, 1.02 g, crude) as an off-white solid.

### Step-9: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(tert-butoxycarbonyl)amino)hexyl)piperazine-1-carboxylate (Int-9)

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl piperazine-1-carboxylate (Common Int-A, 350 mg, 0.542 mmol, 1.0 eq) and (8S,11R,13S,14S,17R)-17-acetyl-11-(4-((tert-butoxycarbonyl) (6-oxohexyl)amino)phenyl)-13-methyl-3-oxo-2,3,6,7,8,11,12,13,14,15,16,17-dodecahydro-1H-cyclopenta[a]phenanthren-17-yl acetate TFA salt (Int-8, 0.5969 mmol, 1.1 eq) in methanol (7 mL) were added Triethylamine (0.07 mL, 0.542 mmol, 1 eq). AcOH (0.542 mmol, 1 eq) at room temperature and stirred for 3 h. To this reaction mixture was added NaCNBH₃ (68 mg, 1.085 mmol, 2 eq) portion wise at 0° C. The resultant reaction mixture was allowed to room temperature and stirred for 16 h. Progress of the reaction was monitored by LCMS. After complete consumption of starting materials, the reaction mixture was allowed to room temperature and stirred for 16 h. Progress of the reaction was monitored by TLC. After complete consumption of the starting materials, cold water (50 mL) was added to the reaction mixture and extracted with 10% MeOH in DCM (2×50 mL). The combined organic extracts were washed with water (60 mL), brine (50 mL) and dried over anhydrous sodium sulphate, filtered, and concentrated under reduced pressure to obtain crude compound, which was purified by combi flash chromatography by eluting with 10% Methanol in DCM to afford(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl) (tert-butoxycarbonyl)amino)hexyl)piperazine-1-carboxylate (Int-9, 0.15 g, 20% yield) as an off-white solid.

### Step-10: Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)amino)hexyl)piperazine-1-carboxylate

To a stirred solution of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)(tert-butoxycarbonyl)amino) hexyl)piperazine-1-carboxylate (Int-9, 150 mg, 0.12893 mmol, 1 eq) in dichloromethane (6.00 mL) was added TFA (0.65 mL, 0.96699 mmol, 7.5 eq) at 0° C. The resultant reaction mixture was warmed to room temperature and stirred for 16 h. Progress of the reaction was monitored by LCMS. After complete consumption of starting materials, the volatiles were removed under reduced pressure to obtain crude compound, which was purified by prep. HPLC purification (method by eluting with Mobile phase A: 0.1% FA in water and Mobile phase B: Acetonitrile) to afford formic acid salt of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl 4-(6-((4-((8S,11R,13S,14S,17R)-17-acetoxy-17-acetyl-13-methyl-3-oxo-2,3,6,7,8,11,12,13,14,15,16,17-dodecahydro-1H-cyclopenta[a]phenanthren-11-yl)phenyl)amino)hexyl)piperazine-1-carboxylate (10 mg, 8% yield) as an off-white solid.

¹H NMR (400 MHz, DMSO-d₆) δ ppm 9.85 (br d, J=3.38 Hz, 2H) 9.11 (s, 1H) 8.37 (br d, J=8.76 Hz, 1H) 7.63-8.00 (m, 1H) 7.38 (s, 1H) 7.04-7.23 (m, 1H) 6.88-7.00 (m, 2H) 6.46-6.59 (m, 3H) 5.68 (s, 1H) 5.45 (s, 2H) 5.35 (s, 2H) 4.86 (br d, J=2.13 Hz, 2H) 4.37 (br d, J=5.88 Hz, 2H) 4.01-4.29 (m, 2H) 3.10-3.27 (m, 6H) 2.96 (br d, J=7.00 Hz, 2H) 2.89 (br d, J=1.63 Hz, 6H) 2.72-2.81 (m, 2H) 2.37 (br s, 1H) 2.19-2.23 (m, 4H) 2.16 (br d, J=2.25 Hz, 2H) 2.10 (s, 3H) 2.00 (s, 3H) 1.87-1.94 (m, 3H) 1.63-1.75 (m, 4H) 1.55 (br d, J=5.63 Hz, 3H) 1.33-1.49 (m, 6H) 1.20-1.30 (m, 1H) 0.89 (br t, J=7.32 Hz, 3H) 0.24 (s, 3H) LCMS: 97.06%; ESI MS m/z calcd. For C₆₂H₇₄N₆O₁₀ ([M+H]⁺) 1063.2; found 1063.7 HPLC: 96.44%.

### Biological Example 1

AR binding assay: To assess AR binding, test compound (top dose 10 μM, 4 fold serial dilution, 8 point dose response) and control (progesterone) were transferred to the assay plate. Cytosol from LnCaP cells was added to the plate, followed by addition of radiolabeled ³H-R1881 at a final concentration of 1 nM. The plate was sealed, and the reaction was incubated at 300 rpm at 4° C. for 24 hrs. Radioligand absorption buffer (10 mM Tris-HCl, pH 7.4; 1.5 mM EDTA; 1 mM DTT; 0.25% charcoal; 0.0025% dextran) was then added to the plate, mixed, and incubated at 4° C. for 15 minutes. The plate was then centrifuged at 3000 rpm for 30 minutes at 4° C. The supernatant was transferred to the scint-tube and Tri-carb was used for scintillation counting. The data was analyzed using GraphPadPrism v5.0 and binding IC₅₀ was determined as the concentration where 50% inhibition of radioligand binding was observed.

GR binding assay: To assess GR binding, test compound (top dose 1 μM, 4 fold serial dilution, 8 point dose response) and control (dexamethasone) were transferred to the assay plate. Cytosol from IM-9 cells was added to the plate, followed by addition of radiolabeled ³H-Dexamethasone at a final concentration of 1.5 nM. The plate was sealed, and the reaction incubated at 300 rpm at 4° C. for 24 hrs. Radioligand absorption buffer (10 mM Tris-HCl, pH 7.4; 1.5 mM EDTA; 1 mM DTT; 0.25% charcoal; 0.0025% dextran) was then added to the plate, mixed, and incubated at 4° C. for 15 minutes. The plate was then centrifuged at 3000 pm for 30 minutes at 4° C. The supernatant was transferred to the scint-tube and Tri-carb was used for scintillation counting. The data was analyzed using GraphPadPrism v5.0 and binding IC₅₀ was determined as the concentration where 50% inhibition of radioligand binding was observed.

PR binding assay: Progesterone PR-B receptors from human breast carcinoma T47D cells were used in modified Na2HPO₄/NaH₂PO₄ buffer pH 7.4. Compounds were screened at a range of doses (Top dose 40 nM, 4 fold serial dilution, 8 point dose response) and were dispensed into the assay plate. Supernatant of 1.2×10⁵ cells aliquot was added to the assay plate and was incubated with 0.5 nM [3H] Progesterone for 20 hours at 4° C. Membranes were filtered and washed, the filters were then counted to determine [3H] Progesterone specifically bound. Binding IC₅₀ was determined as the concentration where 50% inhibition of radioligand binding was observed.

ER binding assay: ERα binding was assessed using the LanthaScreen® TR-FRET ER Alpha Competitive Binding kit from ThermoFisherScientific. In this assay, a terbium-labeled anti-GST antibody was used to indirectly label GST-tagged ER Alpha-ligand binding domain (LBD) by binding to its GST tag. Competitive binding to the ER Alpha-LBD (GST) was detected by a test compound's ability to displace a fluorescent ligand (Fluormone™ ES2 Green tracer) from the ER Alpha-LBD (GST), which results in a loss of FRET signal between the Tb-anti-GST antibody and the tracer. To assess ER binding, test compound (top dose 10 μM, 4 fold serial dilution, 8 point dose response) and controls (e.g. estradiol) were transferred to the assay plate. The Fluormone™ ES2 Green tracer (3 nM final concentration with assay buffer) was added to the assay plate. This was followed by addition of a mixture of the ER Alpha-LBD (GST) and terbium anti-GST antibody. After a 2 h incubation period at room temperature, the plate was read on the Envision plate reader and the TR-FRET ratio of 520:495 emissions were calculated and used to determine the IC₅₀ from a dose response curve of the compound.

AR antagonism assay: To evaluate AR antagonist activity, test compound was added to the assay plate (top dose 10 μM, 3 fold serial dilution, 10 point dose response). HEK293 cells stably expressing the full-length androgen receptor were seeded at a density of 20000 cells/well in the assay plate. The assay plate was then incubated at room temperature for 10 minutes and at 37° C. 5% CO₂ for 20 minutes. Testosterone was added to the assay plate at 1 nM final concentration and the assay plate incubated at 37° C., 5% CO₂ for 20 h. After the incubation period. Steady-glo was added to the assay plate and mixed at room temperature for 20 minutes on an orbital shaker, before reading out on the En Vision plate reader.

GR antagonism assay: To evaluate GR antagonist activity, test compound was added to the assay plate (top dose 5 μM, 4 fold serial dilution, 8 point dose response). HEK293 cells stably expressing the ligand binding domain of the glucocorticoid receptor were seeded at a density of 40000 cells/well in the assay plate. The assay plate was then incubated at 37° C., 5% CO₂ for 30 minutes. Dexamethasone was added to the assay plate at 1.5 nM final concentration and the assay plate incubated at 37° C., 5% CO₂ for 20 h. After the incubation period, Dual-glo luciferase reagent was added to the assay plate and mixed at room temperature for 20 minutes on an orbital shaker, before reading out on the En Vision plate reader. 50 μL of Stop & Glo reagent was added to assay plate, mixed at room temperature for 20 minutes, and read on the Envision plate reader.

PR coactivator antagonist assay: Test compound (Top dose 10 μM, 4 fold serial dilution, 8 point dose response) and/or vehicle was incubated with the 2.5 nM Progesterone Receptor (PR)-LBD and coactivator peptide for 30 minutes at RT. Determination of the amount of complex formed was read spectrofluorimetrically (excitation: 337 nm, emission: 520/490 nm). Test compound-induced inhibition of 10 nM progesterone-induced fluorescence response by 50 percent or more (≥50%) indicates receptor antagonist activity.

ER antagonism assay: To evaluate ER antagonist activity, SK-BR-3 cells were seeded at a density of 30000 cells/well in the assay plate. The assay plate was then incubated at 37° C., 5% CO₂ for 24 h. A mixture of ERE plasmid and ER in opti-MEM media was incubated with lipofectamine 3000 in Opti-MEM media and incubated at room temperature for 15 minutes, 10 μL of this transfection mix was added to each well of the assay plate and the assay plate was incubated at 37° C. 5% CO₂ for 24 h. 100 nM β-Estradiol in 10 μL medium or 10 μL medium (control wells) was added to corresponding wells of assay plate and incubated at 37° C. 5% CO₂ for 24 h. After the incubation period, 50 μL of Dual-glo luciferase reagent was added to the assay plate and mixed at room temperature for 20 mins on an orbital shaker, before reading out on the En Vision plate reader. 50 μL of Stop & Glo reagent was added to assay plate, mixed at room temperature for 20 minutes, and read on the Envision plate reader.

Data for certain compounds as tested in the above assays is shown below in Table 2.

TABLE 2

| | | AR | | GR | | PR |
|---|---|---|---|---|---|---|
| | AR | antag- | GR | antag- | PR | antag- |
| No. | binding | onism | binding | onism | binding | onism |
| 1 | c | d | | | | |
| 2 | c | | | | | |
| 3 | b | | a | b | a | a |
| 5 | b | | a | b | a | a |
| 6 | b | | a | b | a | a |
| 7 | b | | a | b | a | a |
| 8 | a | | b | b | a | a |
| 9 | d | | | | | |
| 10 | b | | | | | |
| 11 | a | | | | | |
| 12 | c | | a | b | a | a |
| 13 | b | | | | | |
| 14 | b | | a | b | a | a |
| 15 | a | | | | | |
| 16 | b | | | | | |
| 17 | b | | | | | |
| 18 | b | c | c | c | | |
| 19 | b | d | a | b | a | a |
| 20 | b | d | | | | |
| 21 | a | d | d | d | | |
| 22 | a | c | c | c | | |
| 23 | b | d | d | d | | |
| 24 | b | d | | | a | a |
| 25 | a | d | d | d | | |
| 26 | a | d | d | d | | |
| 27 | a | c | b | d | | |
| 28 | b | | | | | |
| 29 | | | a | a | | |
| 30 | a | d | d | d | a | d |
| 31 | a | d | d | d | a | d |
| 32 | c | c | | | | |
| 33 | a | d | | | | |
| 34 | b | d | a | c | a | a |
| 35 | b | d | | b | a | d |
| 36 | b | c | c | a | a | d |
| 37 | a | c | | b | a | d |
| 38 | a | d | | b | a | d |
| 39 | b | d | | c | a | d |
| 40 | b | d | | | | |
| 41 | b | c | | a | a | d |
| 42 | b | c | | b | a | d |
| 43 | b | d | | | | |
| 44 | b | d | | | | |
| 45 | b | c | | | | |
| 46 | c | d | | | | |
| 47 | b | b | a | c | a | d |
| 48 | b | c | | | | |
| 49 | b | d | | | | |
| 50 | b | d | | | | |
| 51 | a | b | b | b | a | d |
| 52 | b | b | | | | |
| 53 | b | b | | | | |
| 54 | b | c | a | b | | |
| 55 | b | c | a | b | a | d |
| 56 | c | c | | | b | d |
| 57 | b | | a | | | |
| 58 | b | | | | | |
| 59 | c | c | b | | | |
| 60 | b | d | a | | | |
| 61 | b | c | a | | | |

a: IC₅₀ =< 10 nM;
b: IC₅₀ = 10-100 nM;
c: IC₅₀ = 101-1,000 nM;
d: IC₅₀ => 1,000 nM

Cell viability assay: LNCap, 22Rv1, MCF7, MDAMB361, T47D, and IEC6 cells were seeded at a density of 500-2000 cells/well in 96 well plates, and a ‘T0’ (timepoint 0) was included along with the assay plate. The plates were then incubated at 37° C., 5% CO₂ in a cell culture incubator overnight. On the next day, the ‘T0’ plate was assayed using CellTiterGlo (Promega, Inc) according to the manufacturer's instructions. The appropriate compounds were diluted in DMSO and added to the assay plate (final DMSO concentration of 0.1-0.2%) on the next day. The assay plates were incubated for 6 days at 37° C., 5% CO₂. After the incubation period, the plates were assayed using CellTiterGlo (Promega, Inc) according to the manufacturer's instructions and luminescence was read on the En Vision plate reader. Inhibition of the tested compounds was determined by the following formula: Inhibition rate (%)=(1−(RLU compound−RLU day0)/(RLU control−RLU day0))*100%. GraphPadPrism was used to analyze the data and determine GI₅₀/IC₅₀ values.

Data for certain compounds as tested in the above assay is shown below in Table 3.

TABLE 3

| | 22RV1 | LNCap | T47D | IEC-6 |
|---|---|---|---|---|
| No. | (IC₅₀, nM) | (IC₅₀, nM) | (IC₅₀, nM) | (IC₅₀, nM) |
| 1 | d | d | | d |
| 2 | d | d | | |
| 3 | | c | b | d |
| 4 | c | c | | |
| 5 | | c | c | d |
| 6 | | c | b | d |
| 7 | | c | c | d |
| 8 | | c | b | c |
| 9 | b | b | | d |
| 10 | d | d | | d |
| 11 | b | b | | c |
| 12 | | d | d | d |
| 13 | d | d | | d |
| 14 | | c | b | b |
| 15 | b | b | | d |
| 16 | c | c | | d |
| 17 | c | c | | d |
| 18 | b | b | | c |
| 19 | | c | c | d |
| 20 | c | c | | d |
| 21 | c | b | | d |
| 22 | c | b | | |
| 23 | c | b | | |
| 24 | | c | c | |
| 25 | b | a | | |
| 26 | b | a | | |
| 27 | c | b | | |
| 28 | b | b | | |
| 29 | | b | b | c |
| 30 | c | b | | d |
| 31 | c | b | | c |
| 32 | c | c | | d |
| 33 | c | b | | d |
| 34 | c | | c | c |
| 35 | | d | c | d |
| 36 | | b | b | c |
| 37 | | c | b | c |
| 38 | | c | c | d |
| 39 | c | | c | d |
| 40 | | c | b | c |
| 41 | | b | b | c |
| 42 | c | c | | d |
| 43 | c | c | | d |
| 44 | c | c | | d |
| 45 | b | b | | c |
| 46 | c | c | | d |
| 47 | | b | b | c |
| 48 | c | c | | d |
| 49 | b | b | | d |
| 50 | b | b | | c |
| 51 | b | b | | c |
| 52 | b | b | | c |
| 53 | b | b | | c |
| 54 | | b | c | c |
| 55 | | c | c | d |
| 56 | b | b | | c |
| 57 | | c | b | d |
| 58 | b | c | | c |
| 59 | | b | b | |
| 60 | | d | d | |
| 61 | | c | b | |

a: <10 nM;
b: 10-100 nM;
c: 101-1,000 nM;
d: >1,000 nM

What is claimed is:

1. A compound of Formula I, or stereoisomer, mixture of stereoisomers, hydrate, solvate, isotopically enriched analog or pharmaceutically acceptable salt thereof: 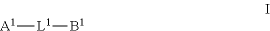
wherein:
B¹ is a nuclear receptor-targeting epitope;
L¹ is a covalent bond or a linking moiety; and
A¹ is of Formula IA: 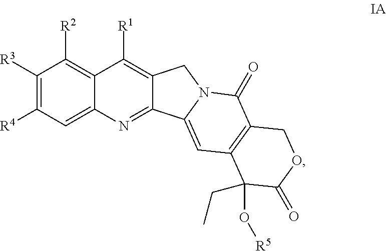
wherein:
R¹, R², R³, R⁴, and R⁵ are each independently hydrogen, halo, cyano, nitro, —OR¹⁵, —SR¹⁵, —NR¹⁵R¹⁶, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁵, —C(═O)OR¹⁵, —OC(═O)R¹⁵, —OC(═O)NR¹⁵R¹⁶, —C(═O)NR¹⁵R¹⁶, —NR¹⁵C(═O)R¹⁶, —NR¹⁵C(═O)OR¹⁶, —S(═O)₁₋₂R¹⁵, —S(═O)₁₋₂NR¹⁵R¹⁶, —NR¹⁵S(═O)₁₋₂R¹⁶, —Si(R¹⁵)₃, or —C═NOR¹⁵, each independently optionally substituted with one or more R¹⁰ as valency permits;
or R¹ and R² are taken together with the atoms to which they are attached to form a C₃₋₁₂ cycloalkyl, 5- to 12-membered heterocyclyl, C₆₋₁₂ aryl, or 5- to 12-membered heteroaryl, each independently optionally substituted with one or more R¹⁰ as valency permits;
or R² and R³ are taken together with the atoms to which they are attached to form a C₃₋₁₂ cycloalkyl, 5- to 12-membered heterocyclyl, C₆₋₁₂ aryl, or 5- to 12-membered heteroaryl, each independently optionally substituted with one or more R¹⁰ as valency permits;
or R³ and R⁴ are taken together with the atoms to which they are attached to form a C₃₋₁₂ cycloalkyl, 5- to 12-membered heterocyclyl, C₆₋₁₂ aryl, or 5- to 12-membered heteroaryl, each independently optionally substituted with one or more R¹⁰ as valency permits;
each R¹⁰ is independently halo, cyano, nitro, —OR¹⁷, —SR¹⁷, —SF₅, —NR¹⁷R¹⁸, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷, —C(═O)OR¹⁷, —OC(═O)OR¹⁷, —OC(═O)R¹⁷, —C(═O)NR¹⁷R¹⁸, —OC(═O)NR¹⁷R¹⁸, —NR⁷C(═O)NR¹⁷R¹⁸, —S(═O)₁₋₂R¹⁷, —S(═O)₁₋₂NR¹⁷R¹⁸, —NR¹⁷S(═O)₁₋₂R¹⁸, —NR¹⁷S(═O)₁₋₂NR¹⁷R¹⁸, —NR¹⁷C(═O)R¹⁸, —NR¹⁷C(═O)OR¹⁸, —Si(R¹⁷)₃, or —C═NOR¹⁷, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
each of R¹⁵ and R¹⁶ is independently hydrogen, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, or 5- to 12-membered heteroaryl, each independently optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
or R¹⁵ and R¹⁶ are taken together with the atoms to which they are attached to form 5- to 12-membered heterocyclyl optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each R¹⁷ and R¹⁸ is independently hydrogen, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, or 5- to 12-membered heteroaryl, each independently optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
or R¹⁷ and R¹⁸ are taken together with the atoms to which they are attached to form 5- to 12-membered heterocyclyl optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
wherein one or more atoms of Formula IA (e.g., hydrogen, methyl, or hydroxyl) is replaced by a direct covalent bond to L¹.

2. The compound of claim 1, or stereoisomer, mixture of stereoisomers, hydrate, solvate, isotopically enriched analog or pharmaceutically acceptable salt thereof, wherein the compound is not a compound selected from the group of compounds in Table 1X, or stereoisomer, mixture of stereoisomers, hydrate, solvate, isotopically enriched analog or pharmaceutically acceptable salt thereof.

3. The compound of claim 1 or 2, or stereoisomer, mixture of stereoisomers, hydrate, solvate, isotopically enriched analog or pharmaceutically acceptable salt thereof, wherein R¹ is hydrogen.

4. The compound of claim 1 or 2, or stereoisomer, mixture of stereoisomers, hydrate, solvate, isotopically enriched analog or pharmaceutically acceptable salt thereof, wherein R¹ is C₁₋₁₂ alkyl, which is optionally substituted with one or more R¹⁰.

5. The compound of claim 1 or 2, or stereoisomer, mixture of stereoisomers, hydrate, solvate, isotopically enriched analog or pharmaceutically acceptable salt thereof, wherein R¹ is —Si(R¹⁵)₃, which is optionally substituted with one or more R¹⁰.

6. The compound of claim 1 or 2, or stereoisomer, mixture of stereoisomers, hydrate, solvate, isotopically enriched analog or pharmaceutically acceptable salt thereof, wherein R¹ is —C═NOR¹⁵, which is optionally substituted with one or more R¹⁰.

7. The compound of claim 1 or 2, or stereoisomer, mixture of stereoisomers, hydrate, solvate, isotopically enriched analog or pharmaceutically acceptable salt thereof, wherein R¹ is ethyl.

8. The compound of claim 1 or 2, or stereoisomer, mixture of stereoisomers, hydrate, solvate, isotopically enriched analog or pharmaceutically acceptable salt thereof, wherein R¹ is

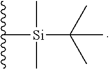 [SMILES: *[Si](C)(C)C(C)(C)C]

9. The compound of claim 1 or 2, or stereoisomer, mixture of stereoisomers, hydrate, solvate, isotopically enriched analog or pharmaceutically acceptable salt thereof, wherein R¹ is

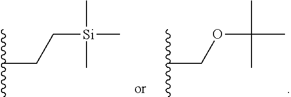

10. The compound of claim 1 or 2, or stereoisomer, mixture of stereoisomers, hydrate, solvate, isotopically enriched analog or pharmaceutically acceptable salt thereof, wherein R¹ is

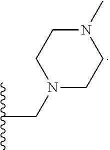 [SMILES: *CN1CCN(C)CC1]

11. The compound of claim 1 or 2, or stereoisomer, mixture of stereoisomers, hydrate, solvate, isotopically enriched analog or pharmaceutically acceptable salt thereof, wherein R¹ is

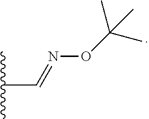 [SMILES: */C=N/OC(C)(C)C]

12. The compound of any one of claims 1-11, or stereoisomer, mixture of stereoisomers, hydrate, solvate, isotopically enriched analog or pharmaceutically acceptable salt thereof, wherein R² is hydrogen.

13. The compound of any one of claims 1-11, or stereoisomer, mixture of stereoisomers, hydrate, solvate, isotopically enriched analog or pharmaceutically acceptable salt thereof, wherein R² is C₁₋₁₂ alkyl optionally substituted with one or more R¹⁰.

14. The compound of any one of claims 1-11, or stereoisomer, mixture of stereoisomers, hydrate, solvate, isotopically enriched analog or pharmaceutically acceptable salt thereof, wherein R² is

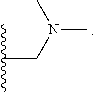 [SMILES: *CN(C)C]

15. The compound of any one of claims 1-11, or stereoisomer, mixture of stereoisomers, hydrate, solvate, isotopically enriched analog or pharmaceutically acceptable salt thereof, wherein R² is nitro.

16. The compound of any one of claims 1-11, or stereoisomer, mixture of stereoisomers, hydrate, solvate, isotopically enriched analog or pharmaceutically acceptable salt thereof, wherein R² is

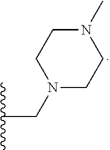 [SMILES: *CN1CCN(C)CC1]

17. The compound of claim 1 or 2, or stereoisomer, mixture of stereoisomers, hydrate, solvate, isotopically enriched analog or pharmaceutically acceptable salt thereof, wherein R¹ and R² are taken together with the atoms to which they are attached to form a C₃₋₁₂ cycloalkyl, which is optionally substituted with one or more R¹⁰.

18. The compound of claim 1 or 2, or stereoisomer, mixture of stereoisomers, hydrate, solvate, isotopically enriched analog or pharmaceutically acceptable salt thereof, wherein R¹ and R² are taken together with the atoms to which they are attached to form

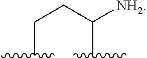 [SMILES: *CCC(*)N]

19. The compound of any one of claims 1-18, or stereoisomer, mixture of stereoisomers, hydrate, solvate, isotopically enriched analog or pharmaceutically acceptable salt thereof, wherein R³ is —OR¹⁵, which is optionally substituted with one or more R¹⁰.

20. The compound of any one of claims 1-18, or stereoisomer, mixture of stereoisomers, hydrate, solvate, isotopically enriched analog or pharmaceutically acceptable salt thereof, wherein R³ is C₁₋₁₂ alkyl, which is optionally substituted with one or more R¹⁰.

21. The compound of any one of claims 1-18, or stereoisomer, mixture of stereoisomers, hydrate, solvate, isotopically enriched analog or pharmaceutically acceptable salt thereof, wherein R³ is —OC(═O)NR¹⁵R¹⁶, which is optionally substituted with one or more R¹⁰.

22. The compound of any one of claims 1-18, or stereoisomer, mixture of stereoisomers, hydrate, solvate, isotopically enriched analog or pharmaceutically acceptable salt thereof, wherein R³ is —OH.

23. The compound of any one of claims 1-18, or stereoisomer, mixture of stereoisomers, hydrate, solvate, isotopically enriched analog or pharmaceutically acceptable salt thereof, wherein R³ is methyl.

24. The compound of any one of claims 1-18, or stereoisomer, mixture of stereoisomers, hydrate, solvate, isotopically enriched analog or pharmaceutically acceptable salt thereof, wherein R³ is

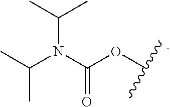 [SMILES: *OC(=O)N(C(C)C)C(C)C]

25. The compound of any one of claims 1-18, or stereoisomer, mixture of stereoisomers, hydrate, solvate, isotopically enriched analog or pharmaceutically acceptable salt thereof, wherein R³ is

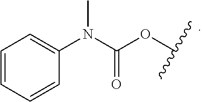 [SMILES: *OC(=O)N(C)c1ccccc1]

26. The compound of any one of claims 1-18, or stereoisomer, mixture of stereoisomers, hydrate, solvate, isotopically enriched analog or pharmaceutically acceptable salt thereof, wherein R³ is

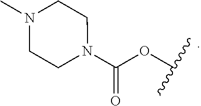 [SMILES: *OC(=O)N1CCN(C)CC1]

27. The compound of any one of claims 1-18, or stereoisomer, mixture of stereoisomers, hydrate, solvate, isotopically enriched analog or pharmaceutically acceptable salt thereof, wherein R³ is

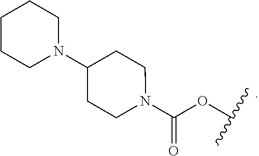 [SMILES: *OC(=O)N1CCC(N2CCCCC2)CC1]

28. The compound of any one of claims 1-18, or stereoisomer, mixture of stereoisomers, hydrate, solvate, isotopically enriched analog or pharmaceutically acceptable salt thereof, wherein R³ is

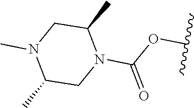 [SMILES: *OC(=O)N1C[C@H](C)N(C)C[C@H]1C]

29. The compound of any one of claims 1-18, or stereoisomer, mixture of stereoisomers, hydrate, solvate, isotopically enriched analog or pharmaceutically acceptable salt thereof, wherein R³ is methoxy.

30. The compound of any one of claims 1-18, or stereoisomer, mixture of stereoisomers, hydrate, solvate, isotopically enriched analog or pharmaceutically acceptable salt thereof, wherein R³ is hydrogen.

31. The compound of any one of claims 1-30, or stereoisomer, mixture of stereoisomers, hydrate, solvate, isotopically enriched analog or pharmaceutically acceptable salt thereof, wherein R⁴ is hydrogen.

32. The compound of any one of claims 1-30, or stereoisomer, mixture of stereoisomers, hydrate, solvate, isotopically enriched analog or pharmaceutically acceptable salt thereof, wherein R⁴ is halo.

33. The compound of any one of claims 1-18, or stereoisomer, mixture of stereoisomers, hydrate, solvate, isotopically enriched analog or pharmaceutically acceptable salt thereof, wherein R³ and R⁴ are taken together with the atoms to which they are attached to form a 5- to 12-membered heterocyclyl, which is optionally substituted with one or more R¹⁰.

34. The compound of any one of claims 1-18, or stereoisomer, mixture of stereoisomers, hydrate, solvate, isotopically enriched analog or pharmaceutically acceptable salt thereof, wherein R³ and R⁴ are taken together with the atoms to which they are attached to form

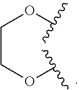 [SMILES: *OCCO*]

35. The compound of any one of claims 1-34, or stereoisomer, mixture of stereoisomers, hydrate, solvate, isotopically enriched analog or pharmaceutically acceptable salt thereof, wherein R⁵ is hydrogen.

36. The compound of any one of claims 1-34, or stereoisomer, mixture of stereoisomers, hydrate, solvate, isotopically enriched analog or pharmaceutically acceptable salt thereof, wherein R⁵ is —C(═O)R¹⁵, which is optionally substituted with one or more R¹⁰.

37. The compound of any one of claims 1-34, or stereoisomer, mixture of stereoisomers, hydrate, solvate, isotopically enriched analog or pharmaceutically acceptable salt thereof, wherein R⁵ is

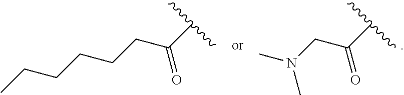

38. The compound of claim 1 or 2, or stereoisomer, mixture of stereoisomers, hydrate, solvate, isotopically enriched analog or pharmaceutically acceptable salt thereof, wherein A¹ is derived from:

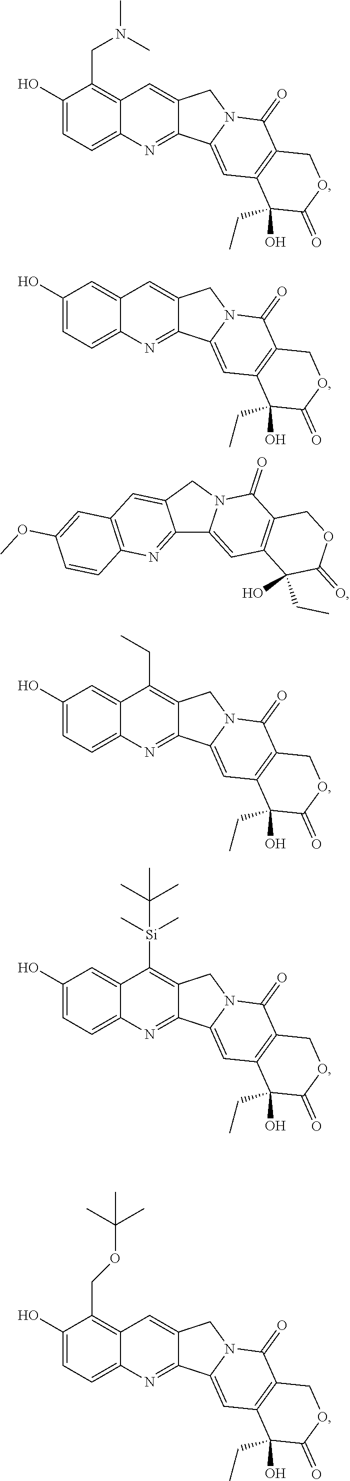

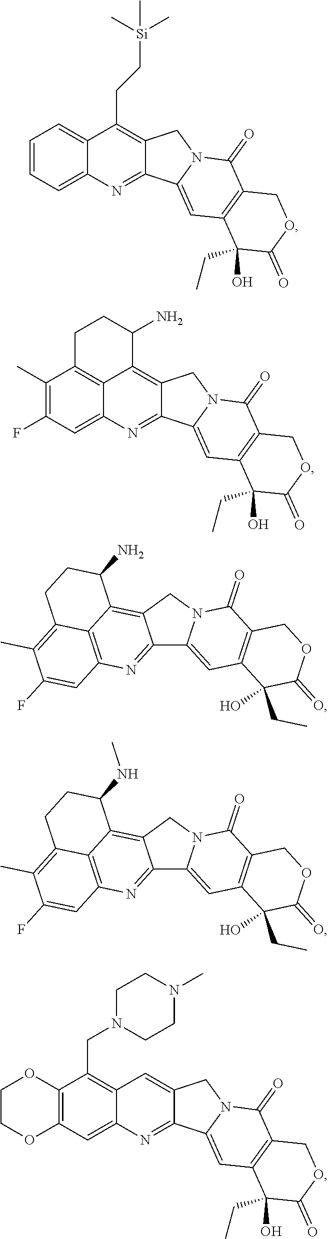

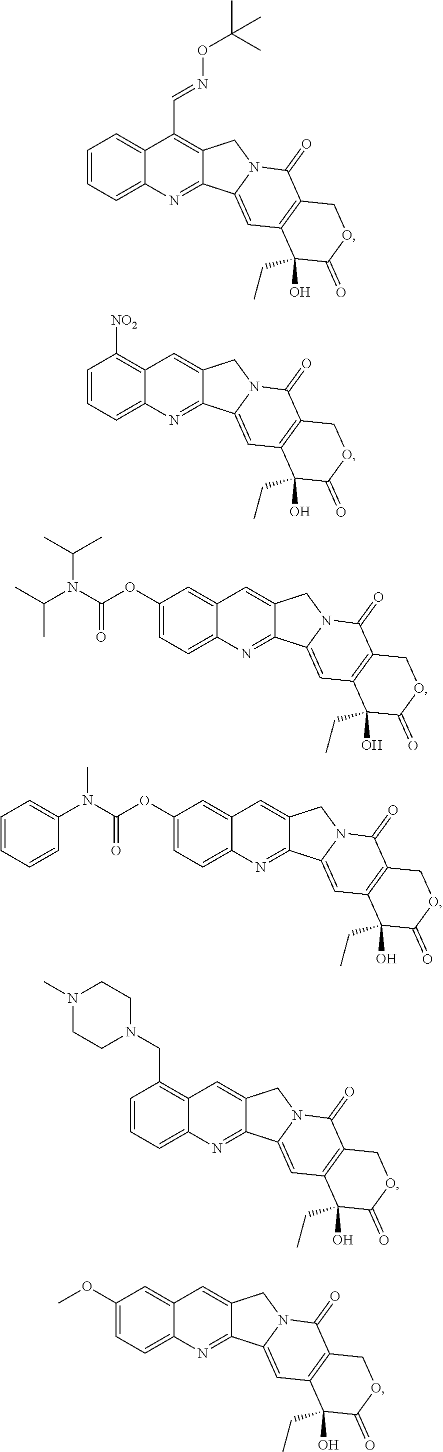 [SMILES: CC[C@@]1(O)C(=O)OCc2c1cc1n(c2=O)Cc2c-1nc1ccccc1c2/C=N/OC(C)(C)C ; CC[C@@]1(O)C(=O)OCc2c1cc1n(c2=O)Cc2cc3c([N+](=O)[O-])cccc3nc2-1 ; CC[C@@]1(O)C(=O)OCc2c1cc1n(c2=O)Cc2cc3cc(OC(=O)N(C(C)C)C(C)C)ccc3nc2-1 ; CC[C@@]1(O)C(=O)OCc2c1cc1n(c2=O)Cc2cc3cc(OC(=O)N(C)c4ccccc4)ccc3nc2-1 ; CC[C@@]1(O)C(=O)OCc2c1cc1n(c2=O)Cc2cc3c(CN4CCN(C)CC4)cccc3nc2-1 ; CC[C@@]1(O)C(=O)OCc2c1cc1n(c2=O)Cc2cc3cc(OC)ccc3nc2-1]

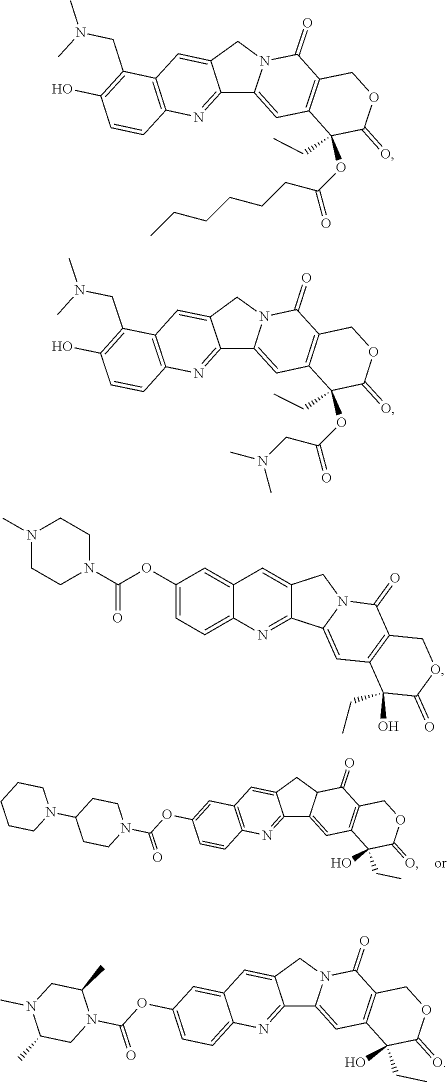

39. The compound of claim 1 or 2, or stereoisomer, mixture of stereoisomers, hydrate, solvate, isotopically enriched analog or pharmaceutically acceptable salt thereof, wherein A¹ is derived from:

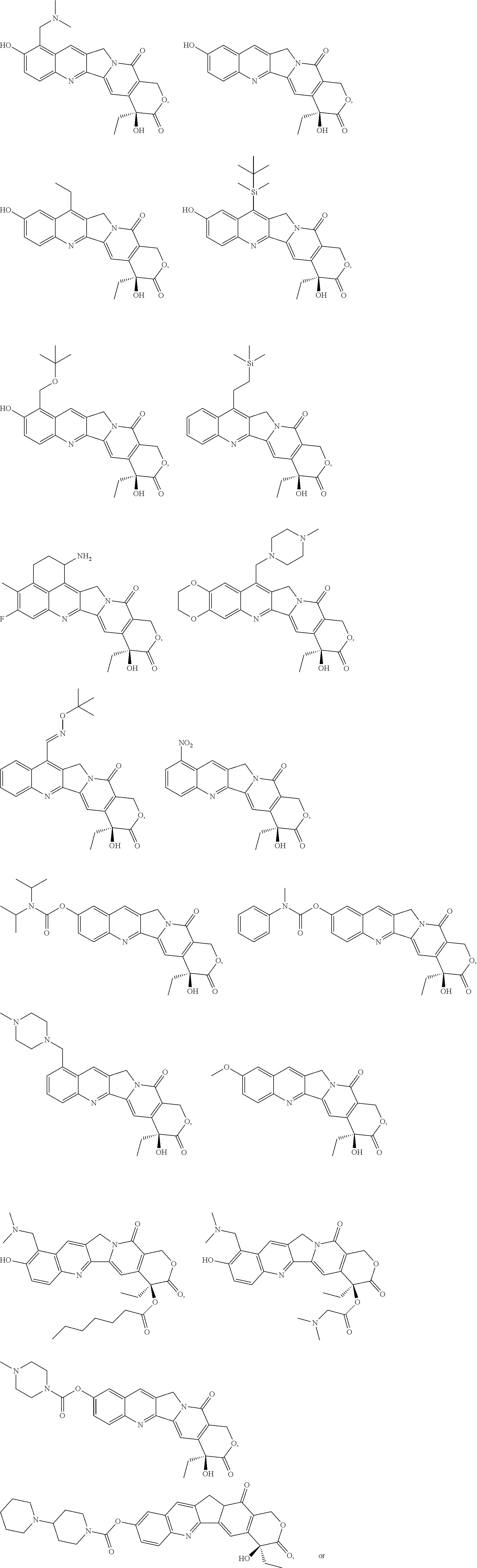

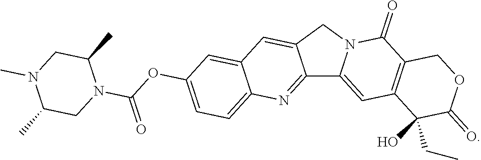 [SMILES: CC[C@@]1(O)C(=O)OCc2c1cc1n(c2=O)Cc2cc3cc(OC(=O)N4C[C@H](C)N(C)C[C@H]4C)ccc3nc2-1]

40. The compound of any one of claims 1-39, or stereoisomer, mixture of stereoisomers, hydrate, solvate, isotopically enriched analog or pharmaceutically acceptable salt thereof, wherein a hydrogen atom of Formula IA is replaced by a direct covalent bond to L¹.

41. The compound of any one of claims 1-39, or stereoisomer, mixture of stereoisomers, hydrate, solvate, isotopically enriched analog or pharmaceutically acceptable salt thereof, wherein a methyl of Formula IA is replaced by a direct covalent bond to L¹.

42. The compound of any one of claims 1-39, or stereoisomer, mixture of stereoisomers, hydrate, solvate, isotopically enriched analog or pharmaceutically acceptable salt thereof, wherein a hydroxyl of Formula IA is replaced by a direct covalent bond to L¹.

43. The compound of any one of claims 1-39, or stereoisomer, mixture of stereoisomers, hydrate, solvate, isotopically enriched analog or pharmaceutically acceptable salt thereof, wherein L¹ is linked to a nitrogen atom of A¹.

44. The compound of any one of claims 1-39, or stereoisomer, mixture of stereoisomers, hydrate, solvate, isotopically enriched analog or pharmaceutically acceptable salt thereof, wherein L¹ is linked to an oxygen atom of A¹.

45. The compound of claim 1 or 2, or stereoisomer, mixture of stereoisomers, hydrate, solvate, isotopically enriched analog or pharmaceutically acceptable salt thereof, wherein A¹ is:

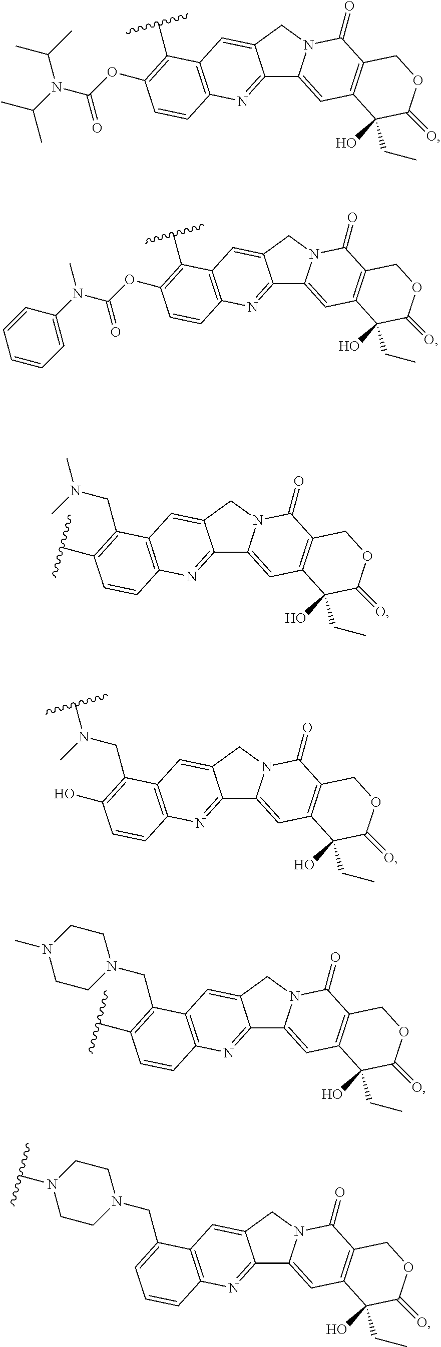 [SMILES: *c1c(OC(=O)N(C(C)C)C(C)C)ccc2nc3c(cc12)Cn1c-3cc2c(c1=O)COC(=O)[C@]2(O)CC ; *c1c(OC(=O)N(C)c2ccccc2)ccc2nc3c(cc12)Cn1c-3cc2c(c1=O)COC(=O)[C@]2(O)CC ; *c1ccc2nc3c(cc2c1CN(C)C)Cn1c-3cc2c(c1=O)COC(=O)[C@]2(O)CC ; *N(C)Cc1c(O)ccc2nc3c(cc12)Cn1c-3cc2c(c1=O)COC(=O)[C@]2(O)CC ; *c1ccc2nc3c(cc2c1CN1CCN(C)CC1)Cn1c-3cc2c(c1=O)COC(=O)[C@]2(O)CC ; *N1CCN(Cc2cccc3nc4c(cc23)Cn2c-4cc3c(c2=O)COC(=O)[C@]3(O)CC)CC1]

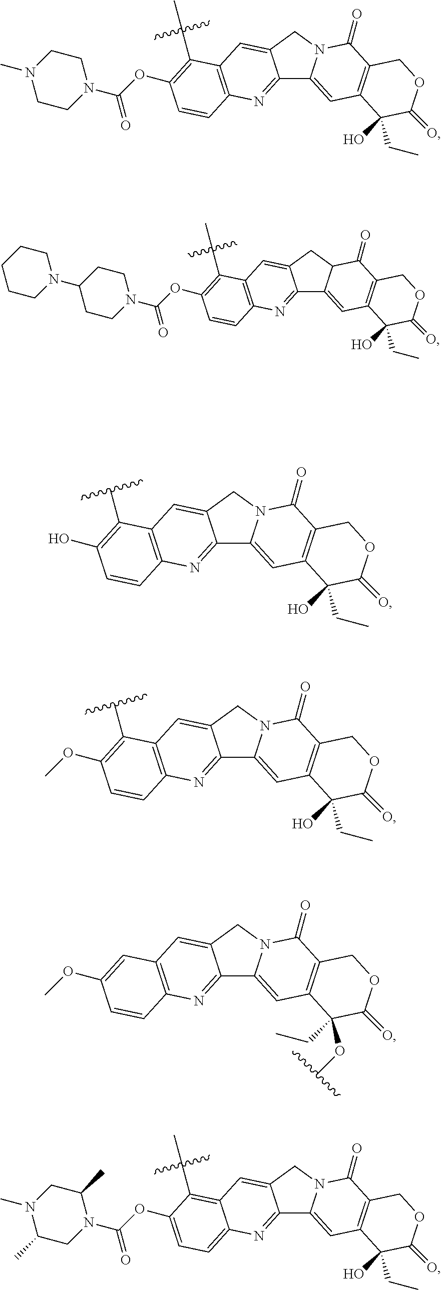 [SMILES: *c1c(OC(=O)N2CCN(C)CC2)ccc2nc3c(cc12)Cn1c-3cc2c(c1=O)COC(=O)[C@]2(O)CC ; *c1c(OC(=O)N2CCC(N3CCCCC3)CC2)ccc2nc3c(cc12)CC1C(=O)C2=C(C=C31)[C@@](O)(CC)C(=O)OC2 ; *c1c(O)ccc2nc3c(cc12)Cn1c-3cc2c(c1=O)COC(=O)[C@]2(O)CC ; *c1c(OC)ccc2nc3c(cc12)Cn1c-3cc2c(c1=O)COC(=O)[C@]2(O)CC ; *O[C@]1(CC)C(=O)OCc2c1cc1n(c2=O)Cc2cc3cc(OC)ccc3nc2-1 ; *c1c(OC(=O)N2C[C@H](C)N(C)C[C@H]2C)ccc2nc3c(cc12)Cn1c-3cc2c(c1=O)COC(=O)[C@]2(O)CC]

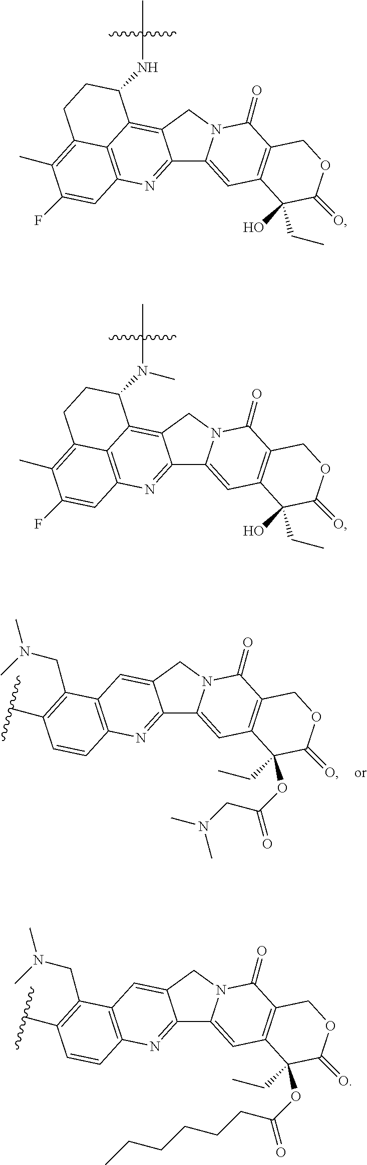 [SMILES: *N[C@H]1CCc2c(C)c(F)cc3nc4c(c1c23)Cn1c-4cc2c(c1=O)COC(=O)[C@]2(O)CC ; *N(C)[C@H]1CCc2c(C)c(F)cc3nc4c(c1c23)Cn1c-4cc2c(c1=O)COC(=O)[C@]2(O)CC ; *c1ccc2nc3c(cc2c1CN(C)C)Cn1c-3cc2c(c1=O)COC(=O)[C@@]2(CC)OC(=O)CN(C)C ; *c1ccc2nc3c(cc2c1CN(C)C)Cn1c-3cc2c(c1=O)COC(=O)[C@@]2(CC)OC(=O)CCCCCC]

46. The compound of claim 1 or 2, or stereoisomer, mixture of stereoisomers, hydrate, solvate, isotopically enriched analog or pharmaceutically acceptable salt thereof, wherein A¹ is:

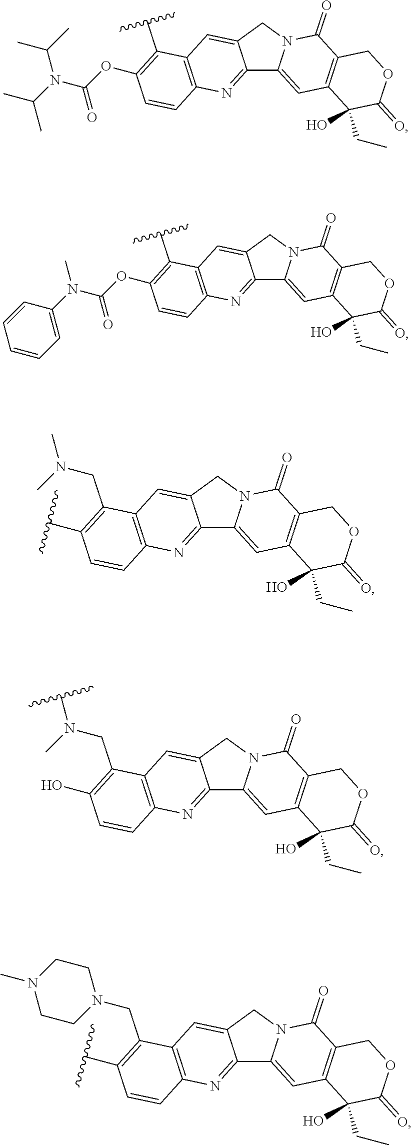 [SMILES: *c1c(OC(=O)N(C(C)C)C(C)C)ccc2nc3c(cc12)Cn1c-3cc2c(c1=O)COC(=O)[C@]2(O)CC ; *c1c(OC(=O)N(C)c2ccccc2)ccc2nc3c(cc12)Cn1c-3cc2c(c1=O)COC(=O)[C@]2(O)CC ; *c1ccc2nc3c(cc2c1CN(C)C)Cn1c-3cc2c(c1=O)COC(=O)[C@]2(O)CC ; *N(C)Cc1c(O)ccc2nc3c(cc12)Cn1c-3cc2c(c1=O)COC(=O)[C@]2(O)CC ; *c1ccc2nc3c(cc2c1CN1CCN(C)CC1)Cn1c-3cc2c(c1=O)COC(=O)[C@]2(O)CC]

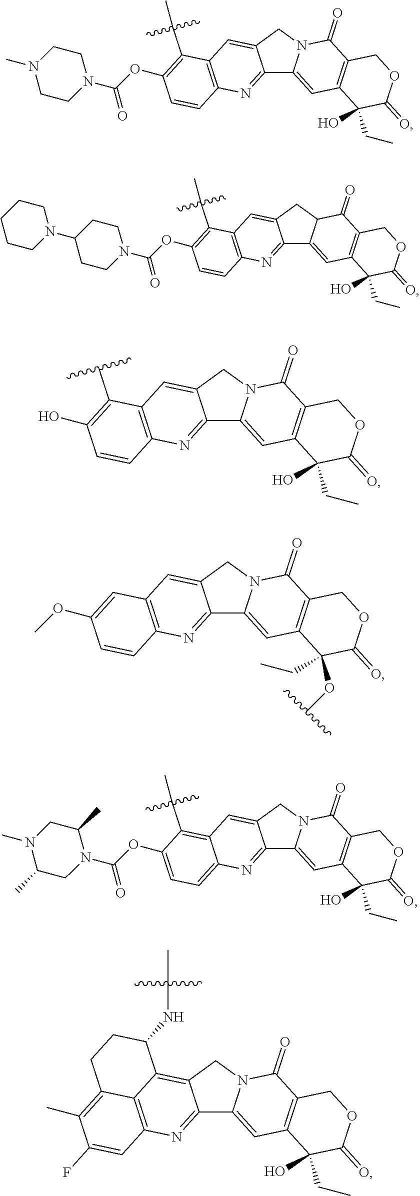 [SMILES: *c1c(OC(=O)N2CCN(C)CC2)ccc2nc3c(cc12)Cn1c-3cc2c(c1=O)COC(=O)[C@]2(O)CC ; *c1c(OC(=O)N2CCC(N3CCCCC3)CC2)ccc2nc3c(cc12)CC1C(=O)C2=C(C=C31)[C@@](O)(CC)C(=O)OC2 ; *c1c(O)ccc2nc3c(cc12)Cn1c-3cc2c(c1=O)COC(=O)[C@]2(O)CC ; *O[C@]1(CC)C(=O)OCc2c1cc1n(c2=O)Cc2cc3cc(OC)ccc3nc2-1 ; *c1c(OC(=O)N2C[C@H](C)N(C)C[C@H]2C)ccc2nc3c(cc12)Cn1c-3cc2c(c1=O)COC(=O)[C@]2(O)CC ; *N[C@H]1CCc2c(C)c(F)cc3nc4c(c1c23)Cn1c-4cc2c(c1=O)COC(=O)[C@]2(O)CC]

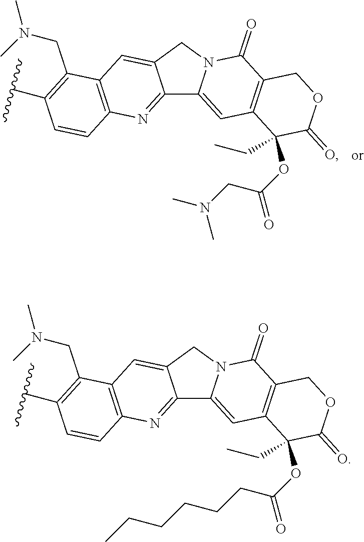 [SMILES: *c1ccc2nc3c(cc2c1CN(C)C)Cn1c-3cc2c(c1=O)COC(=O)[C@@]2(CC)OC(=O)CN(C)C ; *c1ccc2nc3c(cc2c1CN(C)C)Cn1c-3cc2c(c1=O)COC(=O)[C@@]2(CC)OC(=O)CCCCCC]

47. The compound of any one of claims 1-46, or stereoisomer, mixture of stereoisomers, hydrate, solvate, isotopically enriched analog or pharmaceutically acceptable salt thereof, wherein B¹ binds to an estrogen receptor.

48. The compound of any one of claims 1-47, or stereoisomer, mixture of stereoisomers, hydrate, solvate, isotopically enriched analog or pharmaceutically acceptable salt thereof, wherein B¹ binds to a glucocorticoid receptor.

49. The compound of any one of claims 1-48, or stereoisomer, mixture of stereoisomers, hydrate, solvate, isotopically enriched analog or pharmaceutically acceptable salt thereof, wherein B¹ binds to a progesterone receptor.

50. The compound of any one of claims 1-49, or stereoisomer, mixture of stereoisomers, hydrate, solvate, isotopically enriched analog or pharmaceutically acceptable salt thereof, wherein B¹ binds to an androgen receptor.

51. The compound of any one of claims 1-46, or stereoisomer, mixture of stereoisomers, hydrate, solvate, isotopically enriched analog or pharmaceutically acceptable salt thereof, wherein B¹ is of Formula IIA: 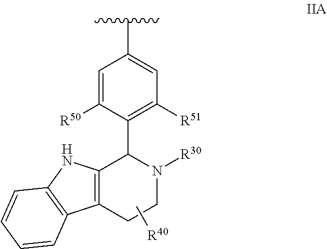
wherein:
the wavy bond represents the point of connection to L¹;
R³⁰ is hydrogen, C₁₋₁₂ alkyl, C₁₋₁₂ haloalkyl, C₂₋₁₂ alkenyl, C₂₋₁₂ alkynyl, or C₃₋₁₂ cycloalkyl, wherein each C₁₋₁₂ alkyl, C₁₋₁₂ haloalkyl, C₂₋₁₂ alkenyl, C₂₋₁₂ alkynyl, or C₃₋₁₂ cycloalkyl is optionally independently substituted with one or more R¹⁰⁰ as valency permits;
R⁴⁰ is hydrogen, C₁₋₁₂ alkyl, C₁₋₁₂ haloalkyl, C₂₋₁₂ alkenyl, C₂₋₁₂ alkynyl, or C₃₋₁₂ cycloalkyl, wherein each C₁₋₁₂ alkyl, C₁₋₁₂ haloalkyl, C₂₋₁₂ alkenyl, C₂₋₁₂ alkynyl, or C₃₋₁₂ cycloalkyl is optionally independently substituted with one or more R¹⁰⁰ as valency permits;
each of R⁵⁰ and R⁵¹ is independently halo, cyano, nitro, —OR¹⁷⁰, —SR¹⁷⁰, —NR¹⁷⁰R¹⁸⁰, C₁₋₁₂ alkyl, C₁₋₁₂ haloalkyl, C₂₋₁₂ alkenyl, or C₂₋₁₂ alkynyl; wherein each C₁₋₁₂ alkyl, C₁₋₁₂ haloalkyl, C₂₋₁₂ alkenyl, or C₂₋₁₂ alkynyl is independently optionally substituted with one or more halo, hydroxyl or amino as valency permits;
each R¹⁰⁰ is independently oxo, halo, cyano, nitro, —OR¹⁷⁰, —SR¹⁷⁰, —SF₅, —NR¹⁷⁰R¹⁸⁰, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷⁰, —C(═O)OR¹⁷⁰, —OC(═O)OR¹⁷⁰, —OC(═O)R¹⁷⁰, —C(═O)NR¹⁷⁰R¹⁸⁰, —OC(═O)NR¹⁷⁰R¹⁸⁰, —NR¹⁷⁰C(═O)NR¹⁷⁰R¹⁸⁰, —S(═O)₁₋₂R¹⁷⁰, —S(═O)₁₋₂NR¹⁷⁰R¹⁸⁰, —NR¹⁷⁰S(═O)₁₋₂R¹⁸⁰, —NR¹⁷⁰S(═O)₁₋₂NR¹⁷⁰R¹⁸⁰, —NR¹⁷⁰C(═O)R¹⁸⁰, or —NR¹⁷⁰C(═O)OR¹⁸⁰, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each of R¹⁷⁰ and R¹⁸⁰ is independently hydrogen or C₁₋₁₂ alkyl optionally substituted with oxo, halo, hydroxyl or amino as valency permits,
or R¹⁷⁰ and R¹⁸⁰ are taken together with the atoms to which they are attached to form heterocyclyl optionally substituted by halo or C₁₋₁₂ alkyl optionally substituted by oxo, halo, hydroxyl or amino.

52. The compound of any one of claims 1-46, or stereoisomer, mixture of stereoisomers, hydrate, solvate, isotopically enriched analog or pharmaceutically acceptable salt thereof, wherein B¹ is

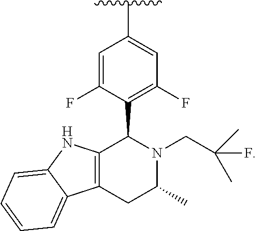 [SMILES: *c1cc(F)c([C@@H]2c3[nH]c4ccccc4c3C[C@@H](C)N2CC(C)(C)F)c(F)c1]

53. The compound of any one of claims 1-46, or stereoisomer, mixture of stereoisomers, hydrate, solvate, isotopically enriched analog or pharmaceutically acceptable salt thereof, wherein B¹ is of Formula IIB′: 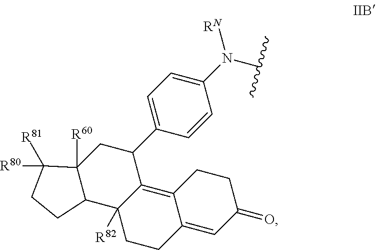
wherein:
the wavy bond represents the point of connection to L¹;
R^{N} is H or C₁₋₁₂ alkyl;
R⁶⁰ is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O) R¹⁰², —NR¹⁰¹C(═O)OR¹⁰², each optionally independently substituted with one or more R¹⁰⁰ as valency permits;
R⁸⁰ is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O)R¹⁰², —NR¹⁰¹C(═O)OR¹⁰², each optionally independently substituted with one or more R¹⁰⁰ as valency permits;
R⁸¹ is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O)R¹⁰², —NR¹⁰¹C(═O)OR¹⁰², each optionally independently substituted with one or more R¹⁰⁰ as valency permits;
or R⁸⁰ and R⁸¹ are taken together with the atom to which they are attached to form heterocyclyl optionally substituted by halo or C₁₋₁₂ alkyl optionally substituted by oxo, halo, hydroxyl or amino;
R⁸² is hydrogen, —OR¹⁰¹, —NR¹⁰¹R¹⁰², C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁰¹, —C(═O)OR¹⁰¹, —OC(═O)R¹⁰¹, —OC(═O)NR¹⁰¹R¹⁰², —C(═O)NR¹⁰¹R¹⁰², —NR¹⁰¹C(═O)R¹⁰², —NR¹⁰¹C(═O)OR¹⁰², each optionally independently substituted with one or more R¹⁰⁰ as valency permits;
each of R¹⁰¹ and R¹⁰² is independently hydrogen, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, or 5- to 12-membered heteroaryl, each independently optionally substituted with one or more substituents selected from the group consisting of halo, cyano, nitro, hydroxyl, amino, C₁₋₁₂ alkoxyl, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits;
each R¹⁰⁰ is independently oxo, halo, cyano, nitro, —OR¹⁷⁰, —SR¹⁷⁰, —SF₅, —NR¹⁷⁰R¹⁸⁰, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷⁰, —C(═O)OR¹⁷⁰, —OC(═O)OR¹⁷⁰, —OC(═O)R¹⁷⁰, —C(═O)NR¹⁷⁰R¹⁸⁰, —OC(═O)NR¹⁷⁰R¹⁸⁰, —NR¹⁷⁰C(═O)NR¹⁷⁰R¹⁸⁰, —S(═O)₁₋₂R¹⁷⁰, —S(═O)₁₋₂NR¹⁷⁰R¹⁸⁰, —NR¹⁷⁰S(═O)₁₋₂R¹⁸⁰, —NR¹⁷⁰S(═O)₁₋₂NR¹⁷⁰R¹⁸⁰, —NR¹⁷⁰C(═O)R¹⁸⁰, or —NR¹⁷⁰C(═O)OR¹⁸⁰, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each of R¹⁷⁰ and R¹⁸⁰ is independently hydrogen or C₁₋₁₂ alkyl optionally substituted with oxo, halo, hydroxyl or amino as valency permits,
or R¹⁷⁰ and R¹⁸⁰ are taken together with the atoms to which they are attached to form heterocyclyl optionally substituted by halo or C₁₋₁₂ alkyl optionally substituted by oxo, halo, hydroxyl or amino.

54. The compound of claim 53, or stereoisomer, mixture of stereoisomers, hydrate, solvate, isotopically enriched analog or pharmaceutically acceptable salt thereof, wherein R^{N} is methyl.

55. The compound of any one of claims 1-46, or stereoisomer, mixture of stereoisomers, hydrate, solvate, isotopically enriched analog or pharmaceutically acceptable salt thereof, wherein B¹ is

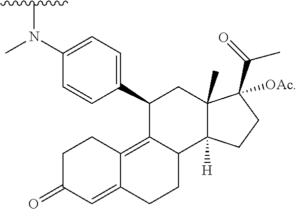 [SMILES: *N(C)c1ccc([C@H]2C[C@@]3(C)[C@@H](CC[C@]3(OC(C)=O)C(C)=O)C3CCC4=CC(=O)CCC4=C32)cc1]

56. The compound of any one of claims 1-46, or stereoisomer, mixture of stereoisomers, hydrate, solvate, isotopically enriched analog or pharmaceutically acceptable salt thereof, wherein B¹ is

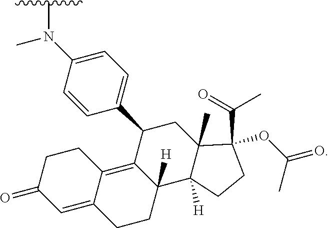

57. The compound of any one of claims 1-46, or stereoisomer, mixture of stereoisomers, hydrate, solvate, isotopically enriched analog or pharmaceutically acceptable salt thereof, wherein B¹ is

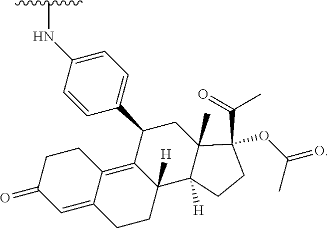

58. The compound of any one of claims 1-46, or stereoisomer, mixture of stereoisomers, hydrate, solvate, isotopically enriched analog or pharmaceutically acceptable salt thereof, wherein B¹ is

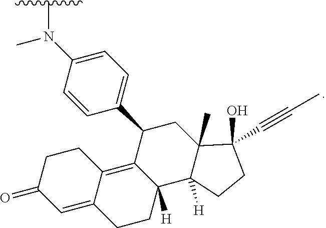

59. The compound of any one of claims 1-46, or stereoisomer, mixture of stereoisomers, hydrate, solvate, isotopically enriched analog or pharmaceutically acceptable salt thereof, wherein B¹ is of Formula IIC′: 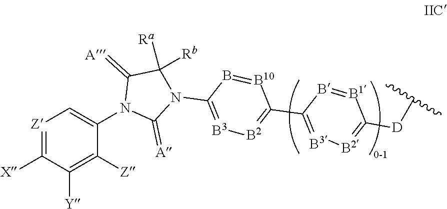
wherein:
the wavy bond represents the point of connection to L¹;
A″ and A′″ are each independently O or S;
R^{a} and R^{b} are each independently CH₃ or CH₂CH₃; or R^{a} and R^{b} together with the atom to which they are attached form a C₃₋₆ cycloalkyl, oxirane, oxetane or tetrahydrofuran;
B, B¹⁰, B², B³, B′, B^{1′}, B^{2′} and B^{3′} are each independently CR^{c} or N;
each R^{c} is independently hydrogen, fluoro, CN, or methyl;
D is absent, NH, O, S, CH₂, —NH(C═O)—, —(C═O)NH—, or C═O;
X″ is CN, halo, or NO₂;
Y″ is CH₃, CH₂R^{d}, CHF₂, or CF₃;
R^{d} is halo;
Z″ is H, C₁₋₂ alkyl, C₂ alkenyl or NO₂; or
X″ and Y″ together form a 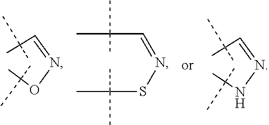
wherein the broken lines indicate bonds to the ring;
or Y″ and Z″ together form a 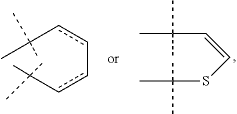
wherein each [symbol] is a single or double bond, and wherein the broken lines indicate bonds to the ring; and
Z′ is CH or N.

60. The compound of claim 59, or stereoisomer, mixture of stereoisomers, hydrate, solvate, isotopically enriched analog or pharmaceutically acceptable salt thereof, wherein D is NH, O, S, CH₂, —NH(C═O)—, —(C═O)NH—, or C═O.

61. The compound of any one of claims 1-46, or stereoisomer, mixture of stereoisomers, hydrate, solvate, isotopically enriched analog or pharmaceutically acceptable salt thereof, wherein B¹ is

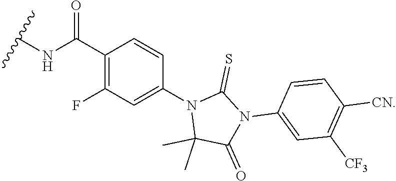

62. The compound of any one of claims 1-46, or stereoisomer, mixture of stereoisomers, hydrate, solvate, isotopically enriched analog or pharmaceutically acceptable salt thereof, wherein B¹ is

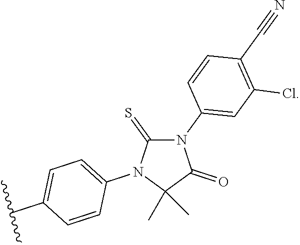 [SMILES: *c1ccc(N2C(=S)N(c3ccc(C#N)c(Cl)c3)C(=O)C2(C)C)cc1]

63. The compound of any one of claims 1-46, or stereoisomer, mixture of stereoisomers, hydrate, solvate, isotopically enriched analog or pharmaceutically acceptable salt thereof, wherein B¹ is of Formula IID′: 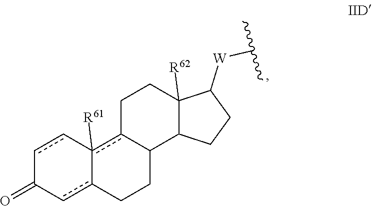
wherein:
W is O, S, or NH;
each [symbol] is independently a double bond or a single bond;
each of R⁶¹ and R⁶² is independently hydrogen, C₁₋₁₂ alkyl, C₂₋₁₂ alkenyl, C₂₋₁₂ alkynyl, or C₃₋₁₂ cycloalkyl, wherein each C₁₋₁₂ alkyl, C₂₋₁₂ alkenyl, C₂₋₁₂ alkynyl, or C₃₋₁₂ cycloalkyl is optionally independently substituted with one or more R¹⁰⁰ as valency permits;
each R¹⁰⁰ is independently oxo, halo, cyano, nitro, —OR¹⁷⁰, —SR¹⁷⁰, —SF₅, —NR¹⁷⁰R¹⁸⁰, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, 5- to 12-membered heteroaryl, —C(═O)R¹⁷⁰, —C(═O)OR¹⁷⁰, —OC(═O)OR¹⁷⁰, —OC(═O)R¹⁷⁰, —C(═O)NR¹⁷⁰R¹⁸⁰, —OC(═O)NR¹⁷⁰R¹⁸⁰, —NR¹⁷⁰C(═O)NR¹⁷⁰R¹⁸⁰, —S(═O)₁₋₂R¹⁷⁰, —S(═O)₁₋₂NR¹⁷⁰R¹⁸⁰, —NR¹⁷⁰S(═O)₁₋₂R¹⁸⁰, —NR¹⁷⁰S(═O)₁₋₂NR¹⁷⁰R¹⁸⁰, —NR¹⁷⁰C(═O)R¹⁸⁰, or —NR¹⁷⁰C(═O)OR¹⁸⁰, each independently optionally substituted with one or more substituents selected from the group consisting of halo, cyano, nitro, hydroxyl, amino, C₁₋₁₂ alkoxy, C₁₋₁₂ alkyl, C₂₋₁₂ alkenyl, C₂₋₁₂ alkynyl, C₃₋₁₂ cycloalkyl, 5- to 12-membered heterocyclyl, C₆₋₁₂ aryl, and 5- to 12-membered heteroaryl, as valency permits; and
each of R¹⁷⁰ and R¹⁸⁰ is independently hydrogen or C₁₋₁₂ alkyl optionally substituted with oxo, halo, hydroxyl or amino as valency permits,
or R¹⁷⁰ and R¹⁸⁰ are taken together with the atoms to which they are attached to form heterocyclyl optionally substituted by halo or C₁₋₁₂ alkyl optionally substituted by oxo, halo, hydroxyl or amino.

64. The compound of claim 63, or stereoisomer, mixture of stereoisomers, hydrate, solvate, isotopically enriched analog or pharmaceutically acceptable salt thereof, wherein B¹ is of Formula IID:

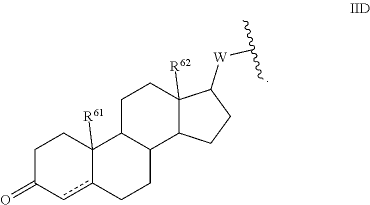

65. The compound of any one of claims 1-46, or stereoisomer, mixture of stereoisomers, hydrate, solvate, isotopically enriched analog or pharmaceutically acceptable salt thereof, wherein B¹ is

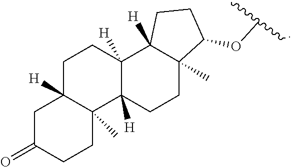 [SMILES: *O[C@H]1CC[C@H]2[C@@H]3CC[C@H]4CC(=O)CC[C@]4(C)[C@H]3CC[C@]12C]

66. The compound of any one of claims 1-46, or stereoisomer, mixture of stereoisomers, hydrate, solvate, isotopically enriched analog or pharmaceutically acceptable salt thereof, wherein B¹ is

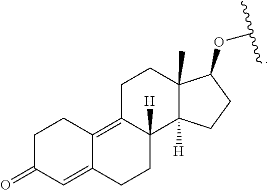

67. The compound of any one of claims 1-46, or stereoisomer, mixture of stereoisomers, hydrate, solvate, isotopically enriched analog or pharmaceutically acceptable salt thereof, wherein B¹ is of Formula IIE: 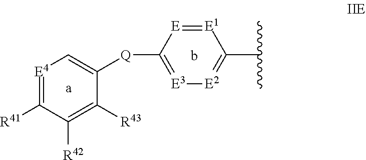
wherein:
the wavy bond refers to the point of connection to L;
Q is 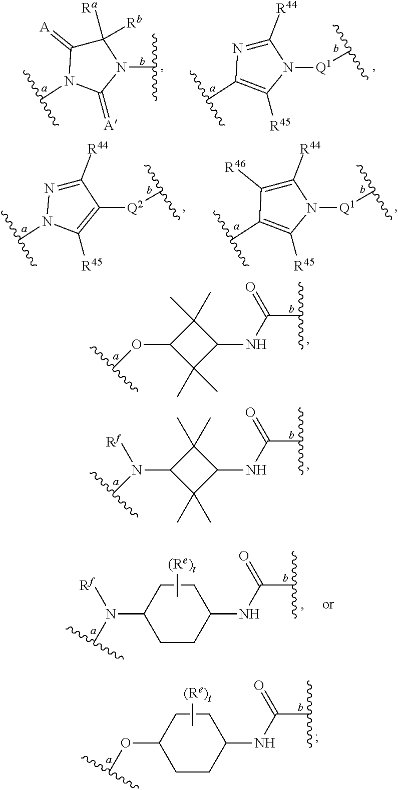
wherein bond a is attached to ring a and bond b is attached to ring b;
R^{a} and R^{b} are each independently —CH₃ or —CH₂CH₃; or R^{a} and R^{b} together with the atom to which they are attached form a C₃₋₅ cycloalkyl, oxiranyl, oxetanyl, or tetrahydrofuranyl;
A and A′ are each independently O or S;
E, E¹, E², and E³ are each independently CR^{c} or N, and each R^{c} is independently hydrogen, halo, CN, or methyl;
E⁴ is CF, CH or N;
Q¹ is a bond, CH₂, C═O, or (C═O)NH;
Q² is NH, O, S, CH₂, NH(C═O), C(═O)NH, or C═O;
R⁴⁴, R⁴⁵ and R⁴⁶ are each independently hydrogen, CN, or C₁₋₂ alkyl;
t is 0, 1, 2, 3 or 4;
each of R^{e} and R^{f} is independently halo, cyano, C₁₋₄ alkyl, or C₁₋₄ haloalkyl;
R⁴¹ is halo, CN, or NO₂;
R⁴² is halo, CH₃, CH₂F, CHF₂, or CF₃; or
R⁴¹ and R⁴² together form a 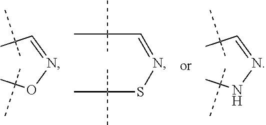
wherein the broken lines indicate bonds to ring a;
R⁴³ is hydrogen, halo, C₁₋₂ alkyl, C₂ alkenyl, NO₂, CF₃; or
R⁴² and R⁴³ together form a 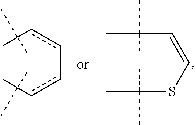
wherein each [symbol] is a single or double bond, and wherein the broken lines indicate bonds to ring a.

68. The compound of any one of claims 1-46, or stereoisomer, mixture of stereoisomers, hydrate, solvate, isotopically enriched analog or pharmaceutically acceptable salt thereof, wherein B¹ is

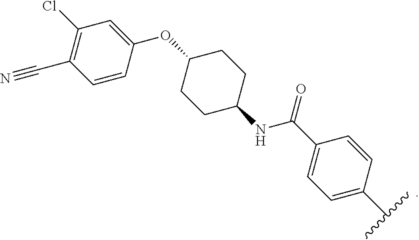 [SMILES: *c1ccc(C(=O)N[C@H]2CC[C@H](Oc3ccc(C#N)c(Cl)c3)CC2)cc1]

69. The compound of any one of claims 1-46, or stereoisomer, mixture of stereoisomers, hydrate, solvate, isotopically enriched analog or pharmaceutically acceptable salt thereof, wherein B¹ is

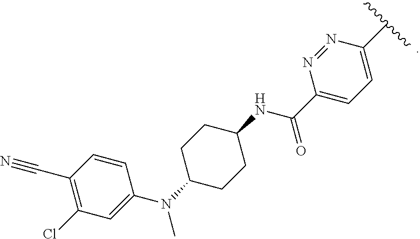 [SMILES: *c1ccc(C(=O)N[C@H]2CC[C@H](N(C)c3ccc(C#N)c(Cl)c3)CC2)nn1]

70. The compound of any one of claims 1-46, or stereoisomer, mixture of stereoisomers, hydrate, solvate, isotopically enriched analog or pharmaceutically acceptable salt thereof, wherein B¹ is

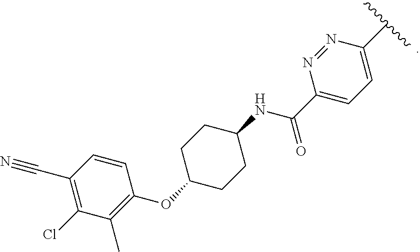 [SMILES: *c1ccc(C(=O)N[C@H]2CC[C@H](Oc3ccc(C#N)c(Cl)c3C)CC2)nn1]

71. The compound of any one of claims 1-46, or stereoisomer, mixture of stereoisomers, hydrate, solvate, isotopically enriched analog or pharmaceutically acceptable salt thereof, wherein B¹ is

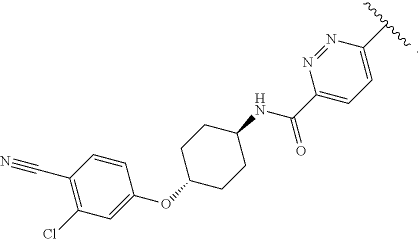 [SMILES: *c1ccc(C(=O)N[C@H]2CC[C@H](Oc3ccc(C#N)c(Cl)c3)CC2)nn1]

72. The compound of any one of claims 1-46, or stereoisomer, mixture of stereoisomers, hydrate, solvate, isotopically enriched analog or pharmaceutically acceptable salt thereof, wherein B¹ is derived from progesterone, enobosarm, bicalutamide, apalutamide, testosterone, dihydrotestosterone, testosterone, 19-nortestosterone, progesterone, andarine, cortisol, prednisone, flutamide, nilutamide, enzalutamide, tamoxifen, toremifene, raloxifene, bazedoxifene, ospemifene, megestrol acetate, estramustine, abiraterone, LGD-2941, BMS-564929, ostarine, ulipristal acetate, asoprisnil (J867), mifepristone, telapristone (CDB-4124, Proellex, Progenta), or an analog thereof.

73. The compound of any one of claims 1-72, or stereoisomer, mixture of stereoisomers, hydrate, solvate, isotopically enriched analog or pharmaceutically acceptable salt thereof, wherein L¹ is of formula: 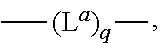
wherein:
each L¹ is independently W, —NR¹¹⁰—, —O—, —S(O)₀₋₂—, —NR¹¹⁰C(O)—, —C(O)NR¹¹⁰—, —NR¹¹⁰C(O)NR¹¹⁰—, —NR¹¹⁰S(O)₂—, —S(O)₂NR¹¹⁰—, —NR¹¹⁰S(O)₂NR¹¹⁰—, —CR¹²⁰═N—NR¹¹⁰—, —NR¹¹⁰—N═CR¹²⁰—, —C(O)—, —OC(O)—, —OC(O)O—, —C(O)O—, C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene, each independently optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl,
wherein each W is independently 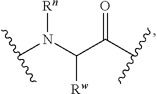
wherein Rⁿ, at each occurrence, is independently H, C₁₋₄ alkyl, or C₁₋₄ haloalkyl, and wherein R^{w}, at each occurrence, is independently H, C₃₋₁₂ cycloalkyl, C₆₋₁₂ aryl optionally substituted with one or more halo or OH, or C₁₋₄ alkyl optionally substituted with one or more independently selected halo, OH, —SH, —S(C₁₋₄ alkyl), —CONH₂, —COOH, —NHC(═NH)NH₂, —NH₂, —NHCOCH₃, —NHCHO, —NHCONH₂, C₆₋₁₂ aryl, 5- to 12-membered heterocycle, or 5- to 12-membered heteroaryl;
each R¹¹⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹²⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl; and
q is an integer from 0 to 40.

74. The compound of any one of claims 1-72, or stereoisomer, mixture of stereoisomers, hydrate, solvate, isotopically enriched analog or pharmaceutically acceptable salt thereof, wherein L¹ is of formula: 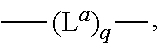
wherein:
each L^{a} is independently —NR¹¹⁰, —O—, —S(O)₀₋₂—, —NR¹¹⁰C(O)—, —C(O)NR¹¹⁰—, —NR¹¹⁰C(O)NR¹¹⁰—, —NR¹¹⁰S(O)₂—, —S(O)₂NR¹¹⁰—, —NR¹¹⁰S(O)₂NR¹¹⁰—, —CR¹²⁰═N—NR¹¹⁰—, —NR¹¹⁰—N═CR¹²⁰—, —C(O)—, —OC(O)—, —OC(O)O—, —C(O)O—, C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene, each independently optionally substituted with one or more substituents independently selected from oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl;
each R¹¹⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹²⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl; and
q is an integer from 0 to 20.

75. The compound of any one of claims 1-72, or stereoisomer, mixture of stereoisomers, hydrate, solvate, isotopically enriched analog or pharmaceutically acceptable salt thereof, wherein L¹ is of the formula: 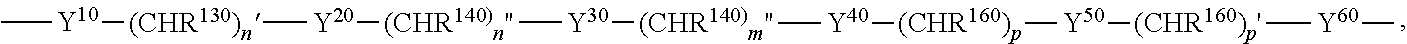
wherein:
each of Y¹⁰, Y²⁰, Y³⁰, Y⁴⁰, Y⁵⁰, and Y⁶⁰ is independently —(W)ₛ—, a bond, —NR¹¹⁰—, —O—, —S(O)₀₋₂—, —NR¹¹⁰C(O)—, —C(O)NR¹¹⁰—, —NR¹¹⁰C(O)NR¹¹⁰—, —NR¹¹⁰S(O)₂—, —S(O)₂NR¹¹⁰—, —NR¹¹⁰S(O)₂NR¹¹⁰—, —CR¹²⁰═N—NR¹¹⁰—, —NR¹¹⁰—N═CR¹²⁰, —C(O)—, —OC(O)—, —OC(O)O—, —(CH₂CH₂O)₁₋₅—, —C(O)O—, C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene, each independently optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl;
each W is independently 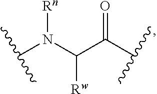
wherein Rⁿ, at each occurrence, is independently H, C₁₋₄ alkyl, or C₁₋₄ haloalkyl; and wherein R^{w}, at each occurrence, is independently H, C₃₋₁₂ cycloalkyl, C₆₋₁₂ aryl optionally substituted with one or more halo or OH, or C₁₋₄ alkyl optionally substituted with one or more independently selected halo, OH, —SH, —S(C₁₋₄ alkyl), —CONH₂, —COOH, —NHC(═NH)NH₂, —NH₂, —NHCOCH₃, —NHCHO, —NHCONH₂, C₆₋₁₂ aryl, 5- to 12-membered heterocycle, or 5- to 12-membered heteroaryl;
each of R¹¹⁰, R¹²⁰, R¹³⁰, R¹⁴⁰, R¹⁵⁰, R¹⁶⁰, and R¹⁷⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl, each independently optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl; and
n′, n″, m″, s, p, and p′ are each independently 0, 1, 2, 3, 4, 5, 6, 7, or 8.

76. The compound of any one of claims 1-72, or stereoisomer, mixture of stereoisomers, hydrate, solvate, isotopically enriched analog or pharmaceutically acceptable salt thereof, wherein L′ is of the formula: 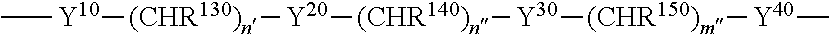
wherein:
each of Y¹⁰, Y²⁰, Y³⁰, and Y⁴⁰ are independently a bond, —NR¹¹⁰—, —O—, —S(O)₀₋₂—, —NR¹¹⁰C(O)—, —C(O)NR¹¹⁰—, —NR¹¹⁰C(O)NR¹¹⁰, —NR¹¹⁰S(O)₂—, —S(O)₂NR¹¹⁰—, —NR¹¹⁰S(O)₂NR¹¹⁰—, —CR¹²⁰═N—NR¹¹⁰—, —NR¹¹⁰—N═CR¹²⁰—,—C(O)—, —OC(O)—, —OC(O)O—, —(CH₂CH₂O)₁₋₅—, —C(O)O—, C₁₋₁₂ alkylene, C₂₋₁₂ alkenylene, C₂₋₁₂ alkynylene, C₆₋₁₂ arylene, C₃₋₁₂ cycloalkylene, 5- to 12-membered heterocyclylene, or 5- to 12-membered heteroarylene, each independently optionally substituted with one or more substituents independently selected from oxo, halo, C₁₋₄ alkyl, C₁₋₄ haloalkyl, C₁₋₄ alkoxy, or C₁₋₄ haloalkoxy;
each R¹¹⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹²⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹³⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹⁴⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl;
each R¹⁵⁰ is independently hydrogen, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, or 5- to 12-membered heterocyclyl; and
n′, n″, and m″ are each independently 0, 1, 2, 3, 4, 5, 6, 7, or 8.

77. The compound of any one of claims 1-72, or stereoisomer, mixture of stereoisomers, hydrate, solvate, isotopically enriched analog or pharmaceutically acceptable salt thereof, wherein L¹ is of the formula: 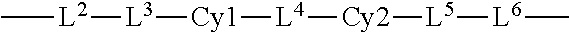
wherein:
each of L², L³, L⁴ L⁵, and L⁶ is independently a bond, C₁₋₁₂ alkylene, —O—, —NHC(═O)—, —C(═O)NH—, —C(═O)—O—, —O—C(═O)—, or C═O, wherein one or more carbon atoms in the C₁₋₁₂ alkylene are optionally replaced with oxygen;
Cy1 and Cy2 are each independently a bond, C₆₋₁₂ arylene, C₃₋₁₂ cycloalkylene, 5- to 12-membered heterocyclylene, or 5- to 12-membered heteroarylene, each of which is independently optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl.

78. The compound of claim 77, or stereoisomer, mixture of stereoisomers, hydrate, solvate, isotopically enriched analog or pharmaceutically acceptable salt thereof, wherein Cy1 is 5- to 12-membered heterocyclylene optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl.

79. The compound of claim 77, or stereoisomer, mixture of stereoisomers, hydrate, solvate, isotopically enriched analog or pharmaceutically acceptable salt thereof, wherein Cy1 is a bond.

80. The compound of any one of claims 77-79, or stereoisomer, mixture of stereoisomers, hydrate, solvate, isotopically enriched analog or pharmaceutically acceptable salt thereof, wherein Cy2 is 5- to 12-membered heterocyclylene optionally substituted with one or more substituents independently selected from —OH, —NH₂, —CN, oxo, halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl.

81. The compound of any one of claims 77-79, or stereoisomer, mixture of stereoisomers, hydrate, solvate, isotopically enriched analog or pharmaceutically acceptable salt thereof, wherein Cy2 is a bond.

82. The compound of any one of claims 1-72, or stereoisomer, mixture of stereoisomers, hydrate, solvate, isotopically enriched analog or pharmaceutically acceptable salt thereof, wherein L¹ is of the formula: 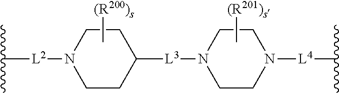
wherein:
each of L², L³, and L⁴ is independently a bond, C₁₋₁₂ alkylene, —NHC(═O)—, —C(═O)NH—, —C(═O)—O—, —O—C(═O)—, or C═O;
each of R²⁰⁰ and R²⁰¹ is independently halo, C₁₋₄ alkyl, C₁₋₄ haloalkyl, C₁₋₄ alkoxy, C₁₋₄ haloalkoxy, C₆₋₁₂ aryl, 5- to 12-membered heteroaryl, C₃₋₁₂ cycloalkyl, and 5- to 12-membered heterocyclyl; and
each of s and s′ is independently 0, 1, 2, 3, or 4.

83. The compound of any one of claims 1-72, or stereoisomer, mixture of stereoisomers, hydrate, solvate, isotopically enriched analog or pharmaceutically acceptable salt thereof, wherein the linking moiety is of the formula: 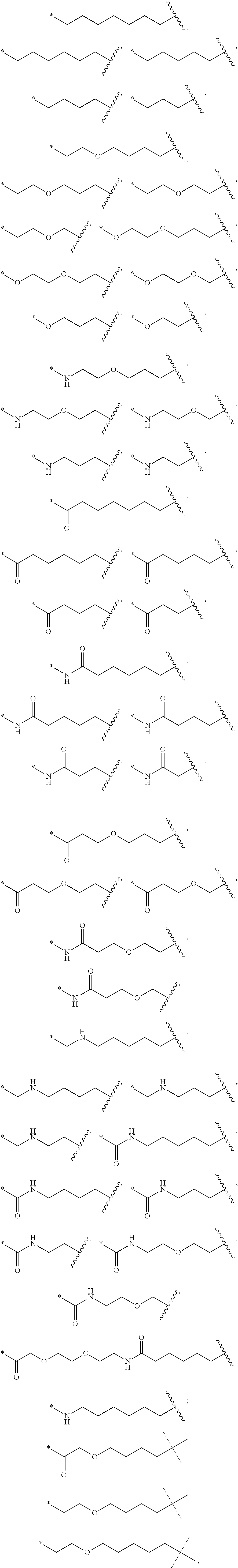 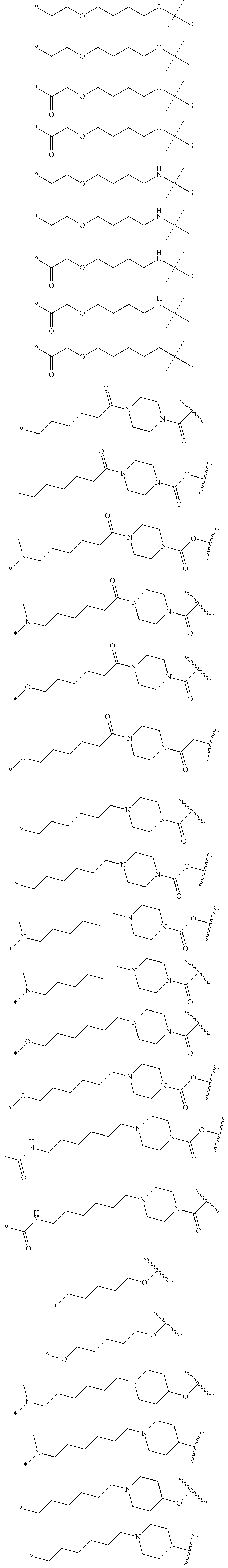 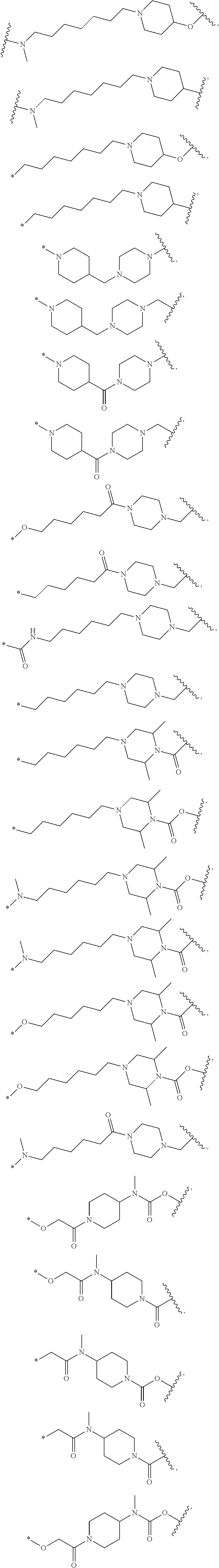 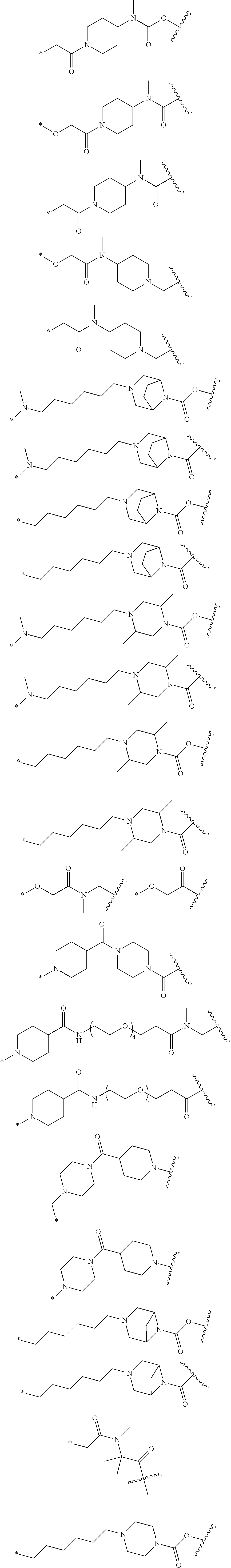 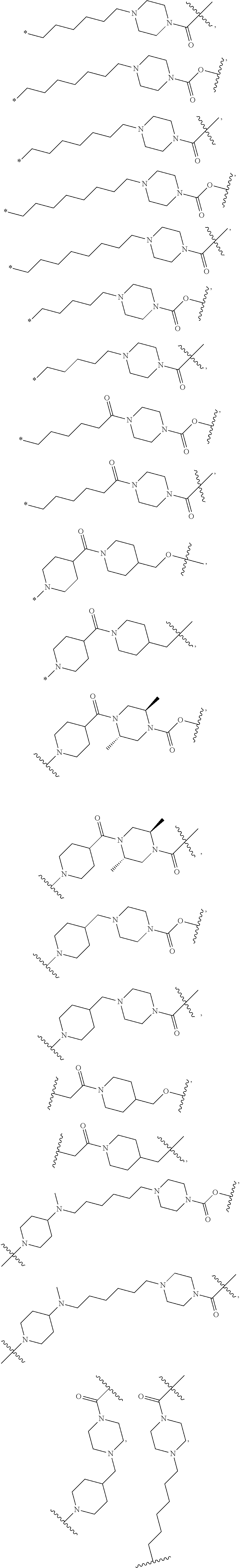 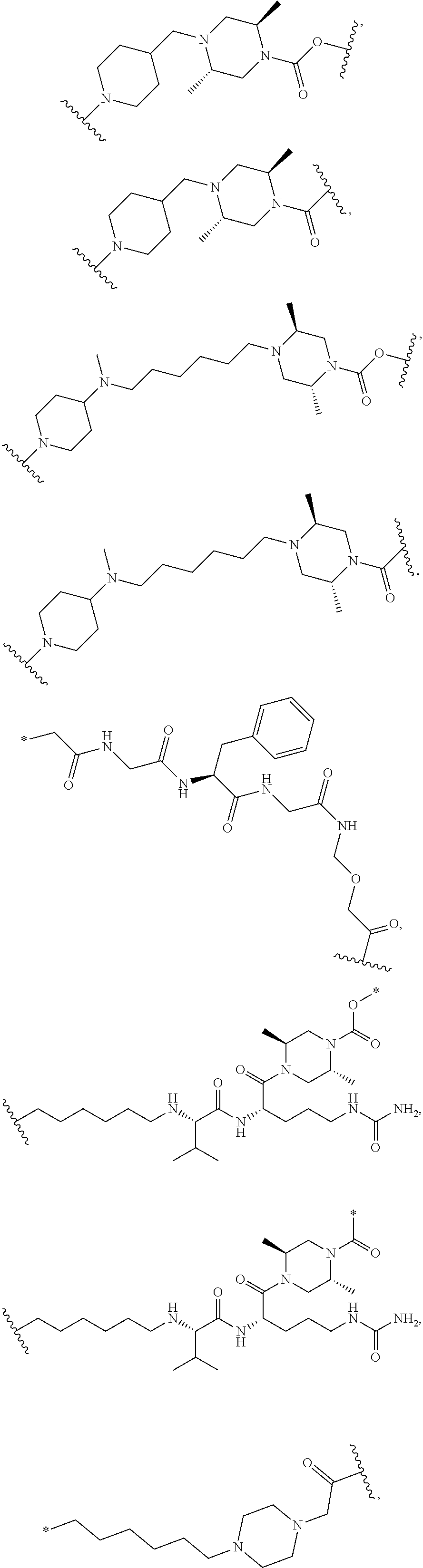 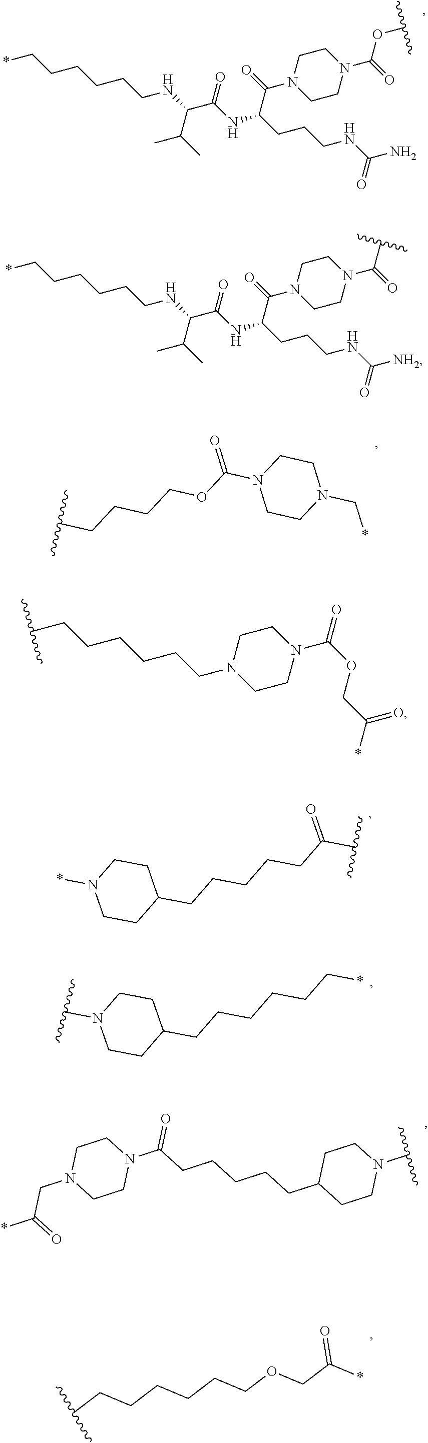 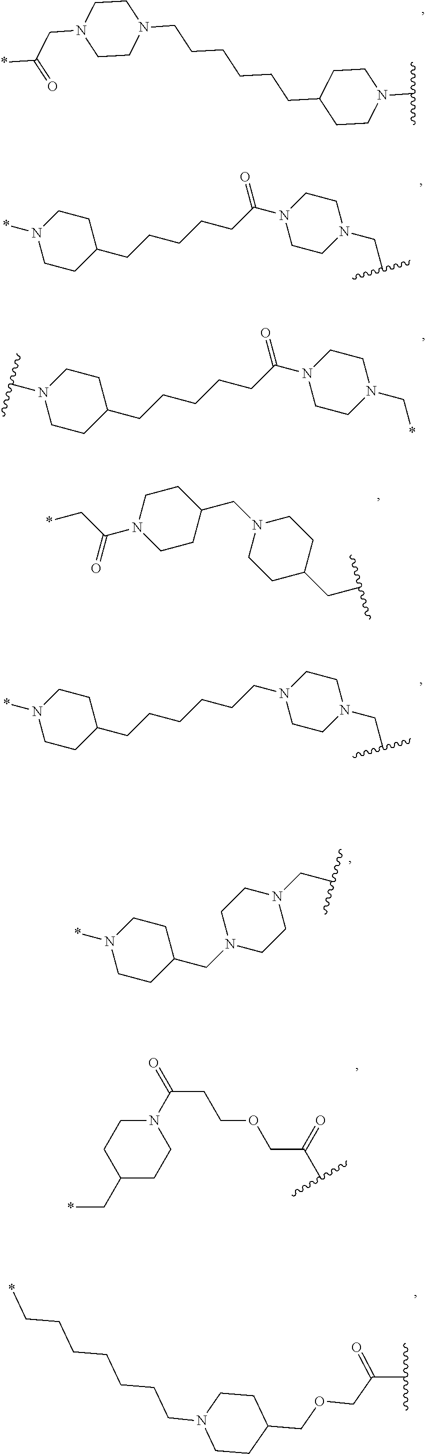 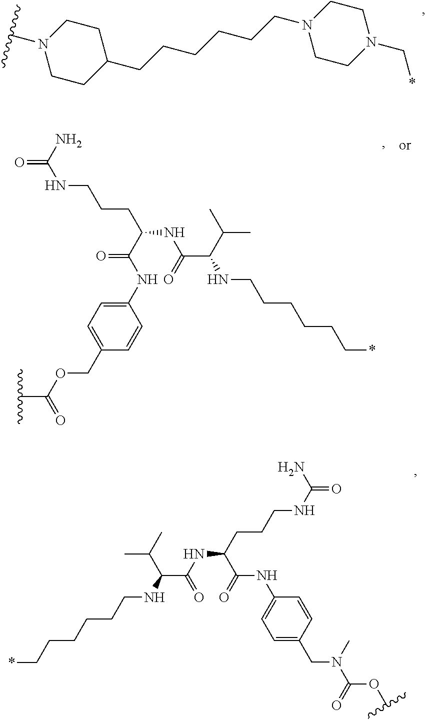 [SMILES: *CN1CCN(CCCCCCC2CCN(*)CC2)CC1 ; *CCCCCCN[C@H](C(=O)N[C@@H](CCCNC(N)=O)C(=O)Nc1ccc(COC(*)=O)cc1)C(C)C ; *CCCCCCN[C@H](C(=O)N[C@@H](CCCNC(N)=O)C(=O)Nc1ccc(CN(C)C(=O)O*)cc1)C(C)C]
wherein the “*” and the wavy or dashed line represent a covalent bond.

84. The compound of any one of claim 1-72 or 82, or stereoisomer, mixture of stereoisomers, hydrate, solvate, isotopically enriched analog or pharmaceutically acceptable salt thereof, wherein the linking moiety is of the formula: 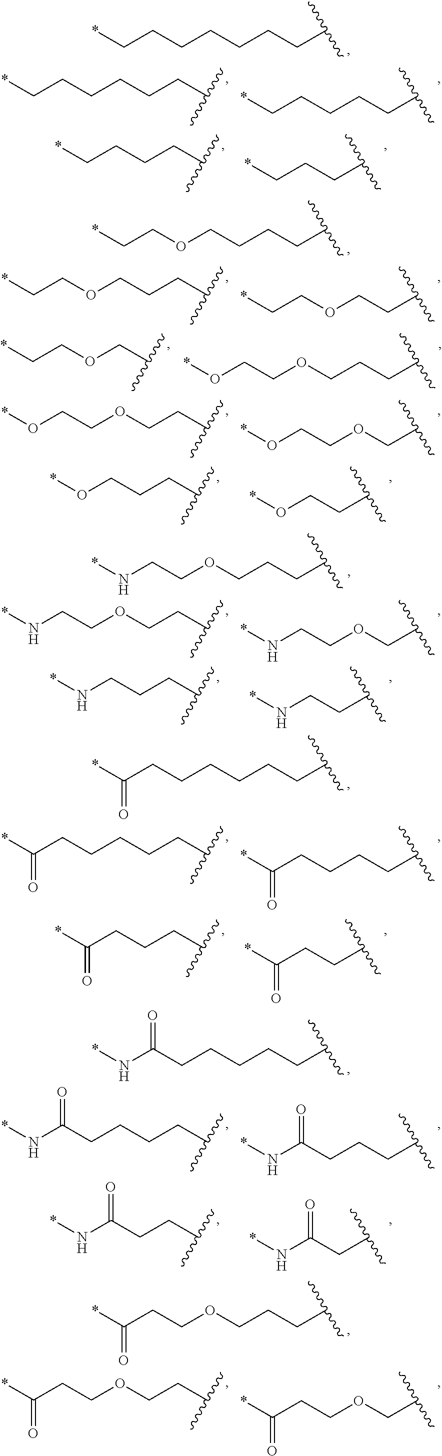 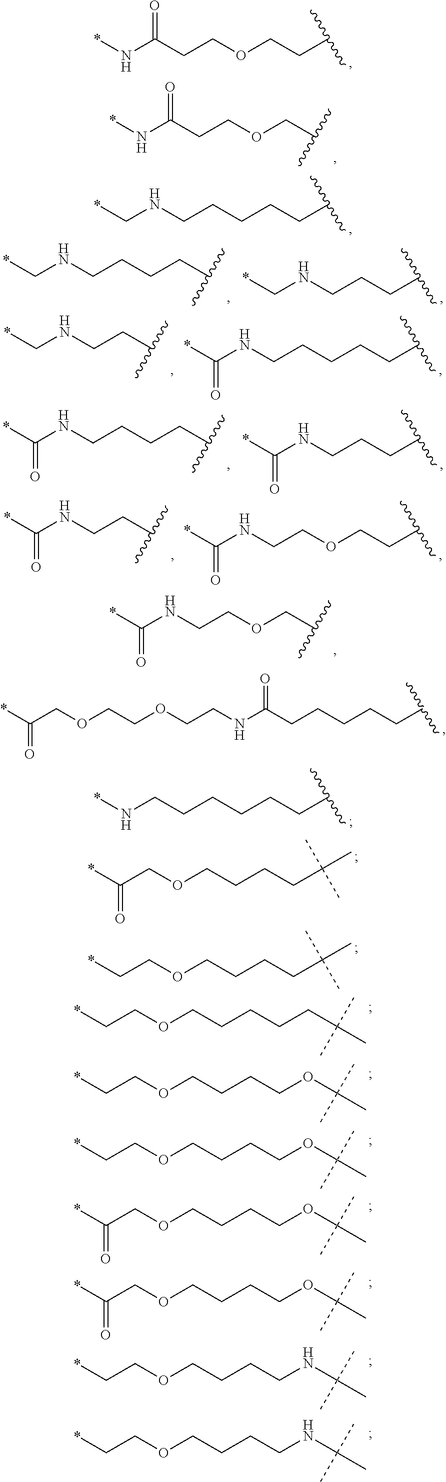 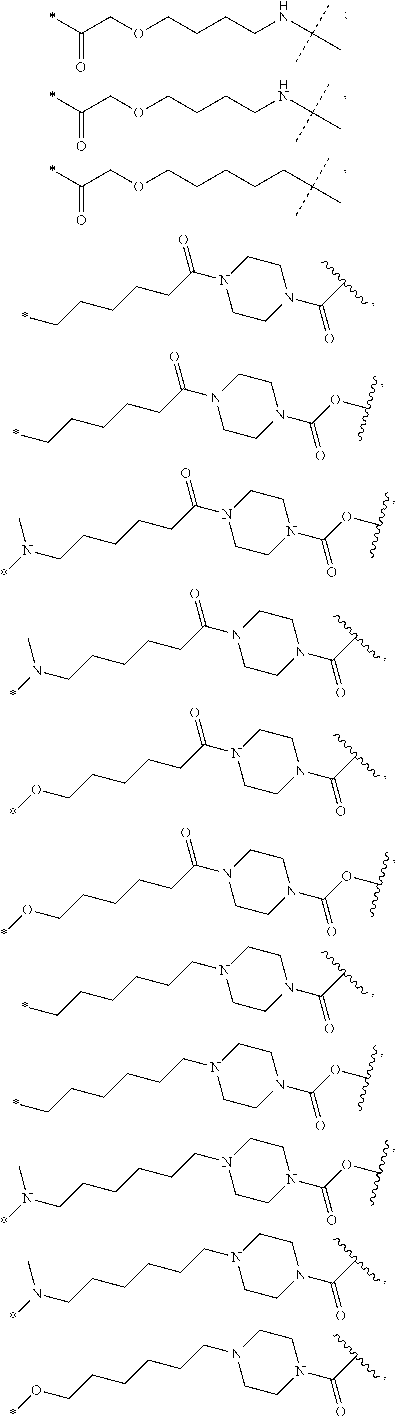 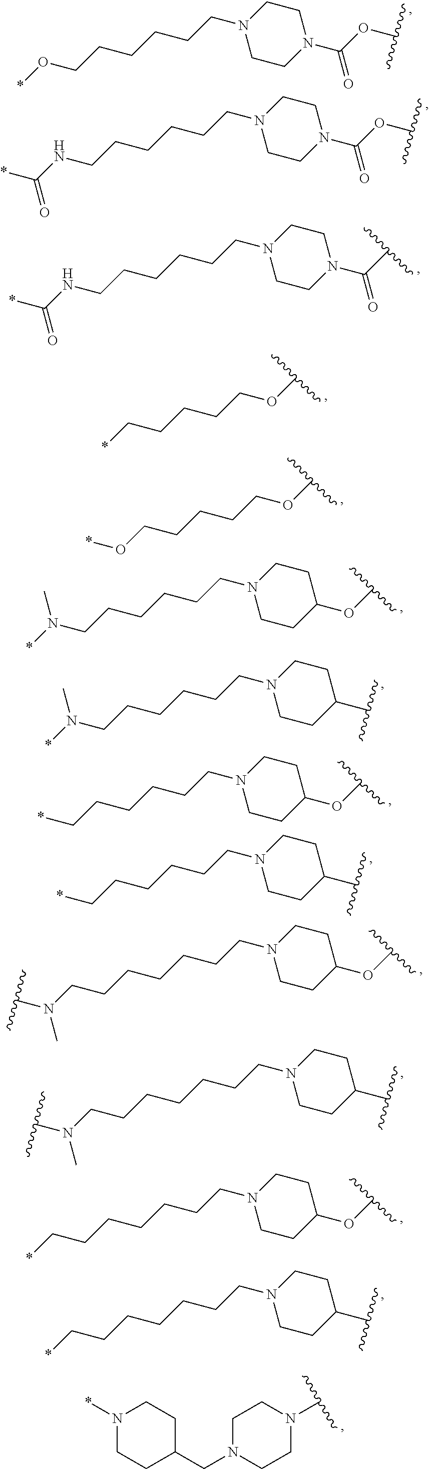 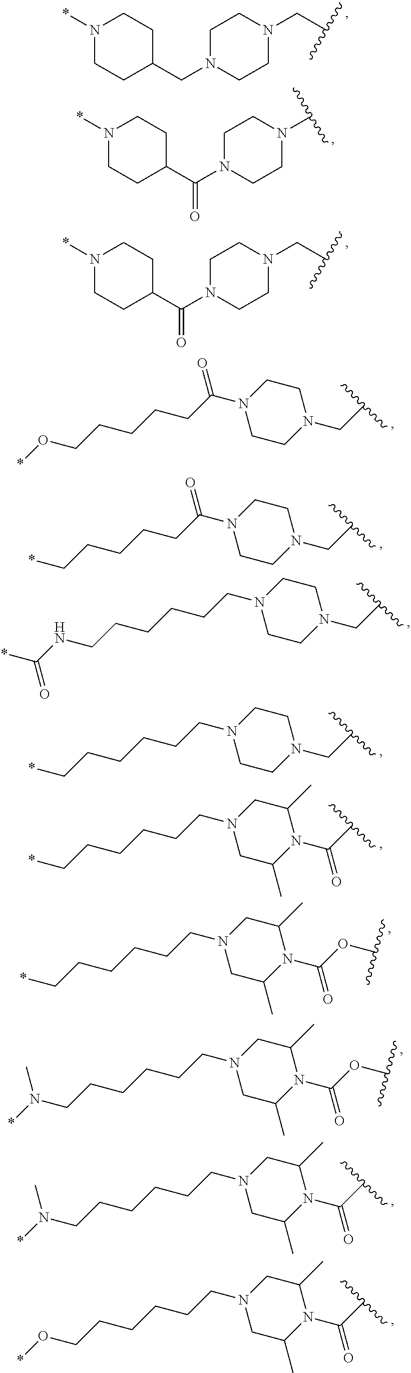 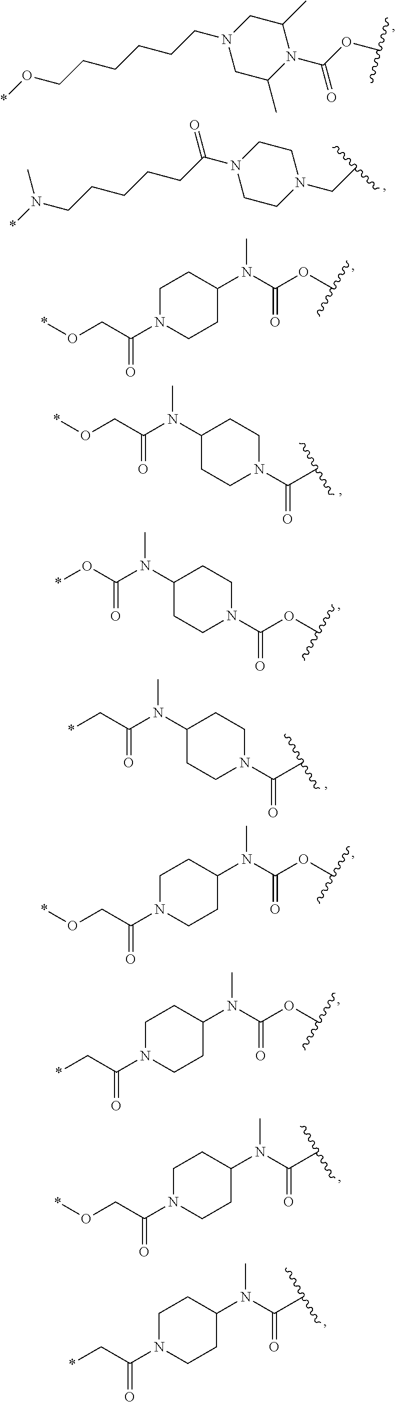 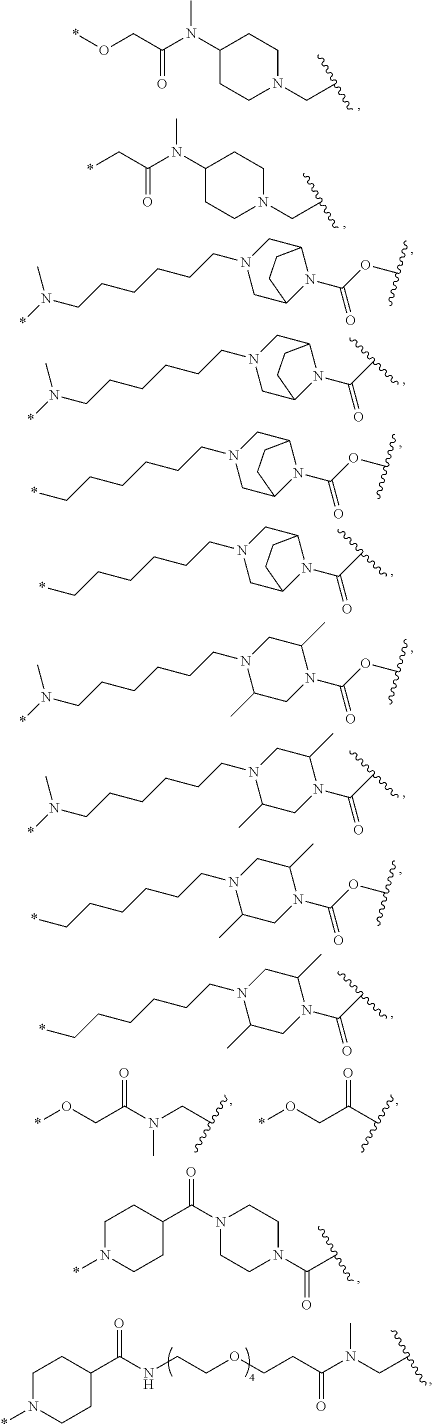 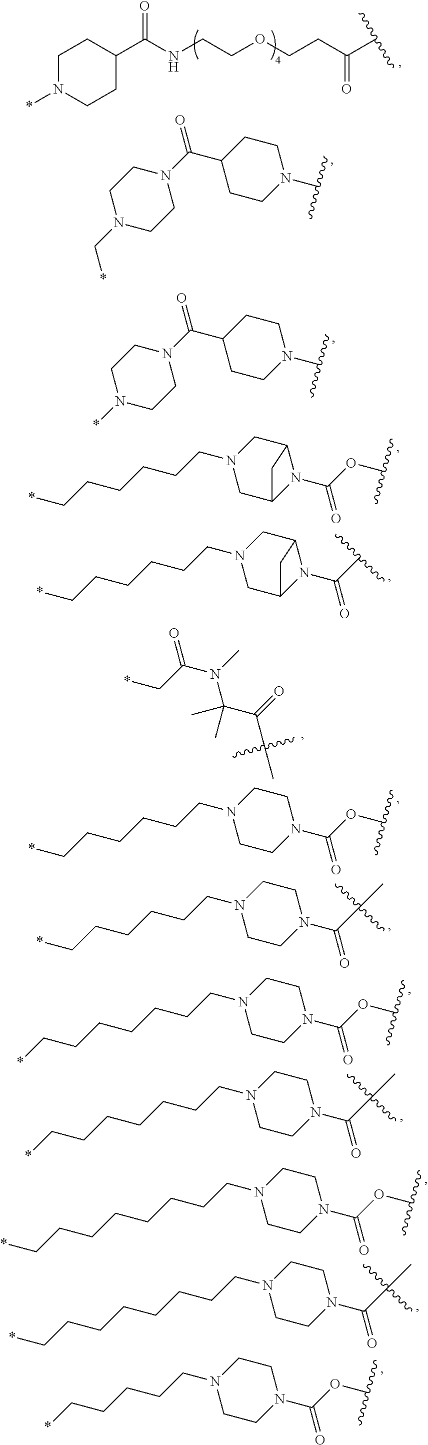 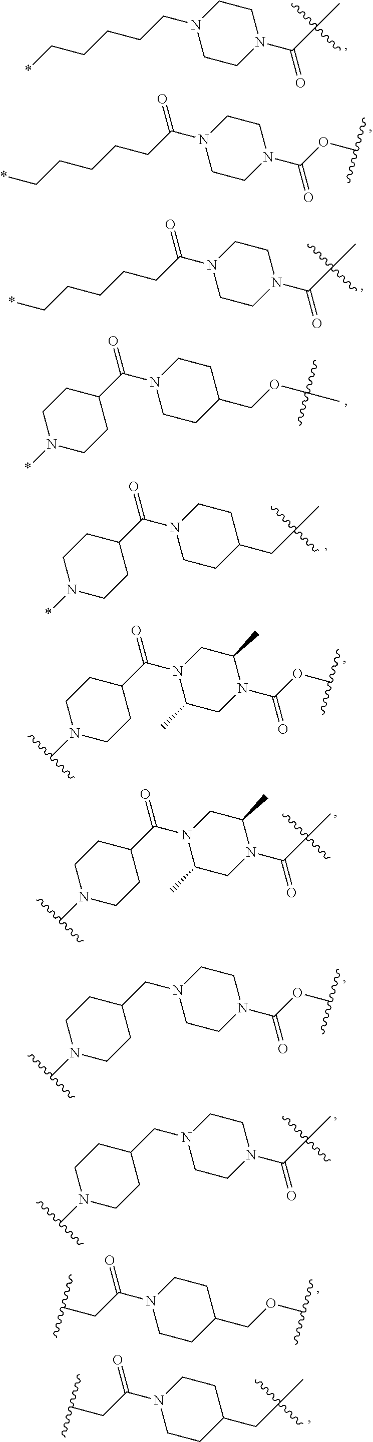 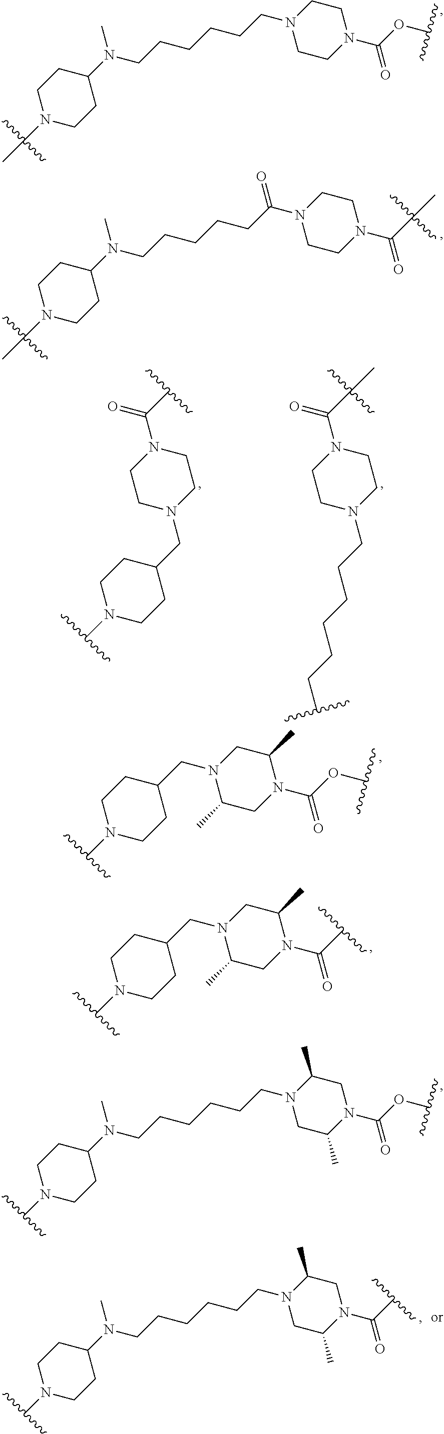 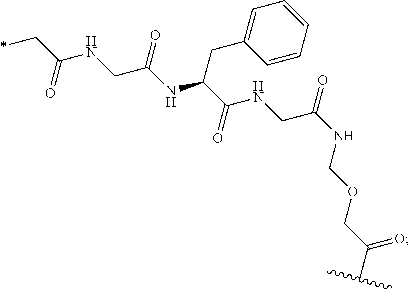 [SMILES: *CC(=O)NCC(=O)N[C@@H](Cc1ccccc1)C(=O)NCC(=O)NCOCC(*)=O]
wherein the “*” and the wavy or dashed line represent a covalent bond.

85. A compound selected from the compounds in Table 1 or a stereoisomer, mixture of stereoisomers, hydrate, solvate, isotopically enriched analog or pharmaceutically acceptable salt thereof.

86. A pharmaceutical composition comprising the compound of any one of claims 1-85 or a stereoisomer, mixture of stereoisomers, hydrate, solvate, isotopically enriched analog or pharmaceutically acceptable salt thereof, and a pharmaceutically acceptable excipient.

87. A method of treating cancer, comprising administering an effective amount of the compound of claims 1-85, or the pharmaceutical composition of claim 86 to an individual in need thereof.

88. The method of claim 87, wherein the cancer is liver cancer, melanoma, Hodgkin's disease, non-Hodgkin's lymphomas, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, Waldenström macroglobulinemia, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, malignant melanoma, choriocarcinoma, mycosis fungoides, head neck carcinoma, osteogenic sarcoma, pancreatic carcinoma, acute granulocytic leukemia, hairy cell leukemia, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, trophoblastic neoplasms, or prostatic carcinoma.
